SEQUENCE LISTING (933 sequences)

SEQ ID NO: 1 - protein, 10 aa, Homo sapiens
GGTFSDYAIS

SEQ ID NO: 2 - protein, 17 aa, Homo sapiens
GIIPISGTANYAQKFQG

SEQ ID NO: 3 - protein, 15 aa, Homo sapiens
DHSSSSYDYQYGLAV

SEQ ID NO: 4 - protein, 5 aa, Homo sapiens
DYAIS

SEQ ID NO: 5 - protein, 17 aa, Homo sapiens
GIIPISGTANYAQKFQG

SEQ ID NO: 6 - protein, 15 aa, Homo sapiens
DHSSSSYDYQYGLAV

SEQ ID NO: 7 - protein, 7 aa, Homo sapiens
GGTFSDY

SEQ ID NO: 8 - protein, 6 aa, Homo sapiens
IPISGT

SEQ ID NO: 9 - protein, 15 aa, Homo sapiens
DHSSSSYDYQYGLAV

SEQ ID NO: 10 - protein, 124 aa, Homo sapiens
QVQLVQSGAEVKKPGSSVKVSCKASGGTFSDYAISVWRQAPGQGLEWMGGIIPISGTANYAQKFQGRVTITADESTSTAYMELSSLRSEDTAVYYCARDHSSSSYDYQYGLAVWGQGTLVTVSS

SEQ ID NO: 11 - DNA, 372 bp, Homo sapiens
caggtgcagctggtgcagtcaggcgccgaagtgaagaaacccggctctagcgtgaaagtcagctgtaaagctagtggcggcacctttagcgactacgctattagctgggtcagacaggccccaggtcagggcctggagtggatgggcggaattatccctattagcggcaccgctaactacgctcagaaatttcagggtagagtgactatcaccgccgacgagtctactagcaccgcctatatggaactgtctagcctgagatcagaggacaccgccgtctactactgcgctagagatcactctagctctagctacgactatcagtacggcctggccgtgtggggtcagggcaccctggtcaccgtgtctagc

SEQ ID NO: 12 - protein, 454 aa, Homo sapiens
QVQLVQSGAEVKKPGSSVKVSCKASGGTFSDYAISVWRQAPGQGLEWMGGIIPISGTANYAQKFQGRVTITADESTSTAYMELSSLRSEDTAVYYCARDHSSSSYDYQYGLAVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWGQGNVFSCSVMHEALHNHYTQKSLSLSPGK

SEQ ID NO: 13 - DNA, 1362 bp, Homo sapiens
caggtgcagctggtgcagtcaggcgccgaagtgaagaaacccggctctagcgtgaaagtcagctgtaaagctagtggcggcacctttagcgactacgctattagctgggtcagacaggccccaggtcagggcctggagtggatgggcggaattatccctattagcggcaccgctaactacgctcagaaatttcagggtagagtgactatcaccgccgacgagtctactagcaccgcctatatggaactgtctagcctgagatcagaggacaccgccgtctactactgcgctagagatcactctagctctagctacgactatcagtacggcctggccgtgtggggtcagggcaccctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 14 - protein, 11 aa, Homo sapiens
SGDKLGDYYVH

SEQ ID NO: 15 - protein, 7 aa, Homo sapiens
QDSKRPS

SEQ ID NO: 16 - protein, 11 aa, Homo sapiens
GATDLSPWSIV

SEQ ID NO: 17 - protein, 11 aa, Homo sapiens
SGDKLGDYYVH

SEQ ID NO: 18 - protein, 7 aa, Homo sapiens
QDSKRPS

SEQ ID NO: 19 - protein, 11 aa, Homo sapiens
GATDLSPWSIV

SEQ ID NO: 20 - protein, 7 aa, Homo sapiens
DKLGDYY

SEQ ID NO: 21 - skipped

SEQ ID NO: 22 - protein, 8 aa, Homo sapiens
TDLSPWSI

SEQ ID NO: 23 - protein, 108 aa, Homo sapiens
DIELTQPPSVSVSPGETASITCSGDKLGDYYVHWYQQKPGGAPVLVIYQDSKRPSGIPERFSGSNSGNTATLTISGTQAEDEADYYCGATDLSPWSIVFGGGTKLTVL

SEQ ID NO: 24 - DNA, 324 bp, Homo sapiens
gatatcgagctgactcagccccctagcgtcagcgtcagccctggcgagacagcctctatcacctgtagcggcgataagctgggcgactactacgtgcactggtatcagcagaagcccggtcaggcccccgtgctggtgatctatcaggactctaagcggcctagcggaatccccgagcggtttagcggctctaatagcggtaacaccgctaccctgactattagcggcactcaggccgaggacgaggccgactactactgcggcgctaccgacctgagcccctggtctatcgtgttcggcggaggcactaagctgaccgtgctg

SEQ ID NO: 25 - protein, 214 aa, Homo sapiens
DIELTQPPSVSVSPGETASITCSGDKLGDYYVHWYQQKPGQAPVLVIYQDSKRPSGIPERFSGSNSGNTATLTISGTQAEDEADYYCGATDLSPWSIVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 26 - DNA, 642 bp, Homo sapiens
gatatcgagctgactcagccccctagcgtcagcgtcagccctggcgagacagcctctatcacctgtagcggcgataagctgggcgactactacgtgcactggtatcagcagaagcccggtcaggcccccgtgctggtgatctatcaggactctaagcggcctagcggaatccccgagcggtttagcggctctaatagcggtaacaccgctaccctgactattagcggcactcaggccgaggacgaggccgactactactgcggcgctaccgacctgagcccctggtctatcgtgttcggcggaggcactaagctgaccgtgctgggtcagcctaaggctgcccccagcgtgaccctgttcccccccagcagcgaggagctgcaggccaacaaggccaccctggtgtgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggaaaagaccgtggccccaaccgagtgcagc

SEQ ID NO: 27 - protein, 10 aa, Homo sapiens
GGTFSDYAIS

SEQ ID NO: 28 - protein, 17 aa, Homo sapiens
GIIPISGTANYAQKFQG

SEQ ID NO: 29 - protein, 15 aa, Homo sapiens
DHSSSSYDYQYGLAV

SEQ ID NO: 30 - protein, 5 aa, Homo sapiens
DYAIS

SEQ ID NO: 31 - protein, 17 aa, Homo sapiens
GIIPISGTANYAQKFQG

SEQ ID NO: 32 - protein, 15 aa, Homo sapiens
DHSSSSYDYQYGLAV

SEQ ID NO: 33 - protein, 7 aa, Homo sapiens
GGTFSDY

SEQ ID NO: 34 - protein, 6 aa, Homo sapiens
IPISGT

SEQ ID NO: 35 - protein, 15 aa, Homo sapiens
DHSSSSYDYQYGLAV

SEQ ID NO: 36 - protein, 124 aa, Homo sapiens
QVQLVQSGAEVKKPGSSVKVSCKASGGTFSDYAISWVRQAPGQGLEWMGGIIPISGTANYAQKFQGRVTITADESTSTAYMELSSLRSEDTAVYYCARDHSSSSYDYQYGLAVWGQGTLVTVSS

SEQ ID NO: 37 - DNA, 372 bp, Homo sapiens
caggtgcaattggtgcagagcggtgccgaagtgaaaaaaccgggcagcagcgtgaaagttagctgcaaagcatccggagggacgttttctgactacgctatctcttgggtgcgccaggccccgggccagggcctcgagtggatgggcggtatcatcccgatctctggcactgcgaactacgcccagaaatttcagggccgggtgaccattaccgccgatgaaagcaccagcaccgcctatatggaactgagcagcctgcgcagcgaagatacggccgtgtattattgcgcgcgtgaccattcttcttcttcttacgactaccagtacggtctggctgtttggggccaaggcaccctggtgactgttagctca

SEQ ID NO: 38 - protein, 454 aa, Homo sapiens
QVQLVGSGAEVKKPGSSVKVSCKASGGTFSDYAISVWRQAPGQGLEWMGGIIPISGTANYAQKFQGRVTITADESTSTAYMELSSLRSEDTAVYYCARDHSSSSYDYQYGLAVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - DNA, 1362 bp, Homo sapiens
caggtgcaattggtgcagagcggtgccgaagtgaaaaaaccgggcagcagcgtgaaagttagctgcaaagcatccggagggacgttttctgactacgctatctcttgggtgcgccaggccccgggccagggcctcgagtggatgggcggtatcatcccgatctctggcactgcgaactacgcccagaaatttcagggccgggtgaccattaccgccgatgaaagcaccagcaccgcctatatggaactgagcagcctgcgcagcgaagatacggccgtgtattattgcgcgcgtgaccattcttcttcttcttacgactaccagtacggtctggctgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 40 - protein, 11 aa, Homo sapiens
SGDKLGDYYVH

SEQ ID NO: 41 - protein, 7 aa, Homo sapiens
QDSKRPS

SEQ ID NO: 42 - protein, 11 aa, Homo sapiens
GATDLSPWSIV

SEQ ID NO: 43 - protein, 11 aa, Homo sapiens
SGDKLGDYYVH

SEQ ID NO: 44 - protein, 7 aa, Homo sapiens
QDSKRPS

SEQ ID NO: 45 - protein, 11 aa, Homo sapiens
GATDLSPWSIV

SEQ ID NO: 46 - protein, 7 aa, Homo sapiens
DKLGDYY

SEQ ID NO: 47 - skipped

SEQ ID NO: 48 - protein, 8 aa, Homo sapiens
TDLSPWSI

SEQ ID NO: 49 - protein, 108 aa, Homo sapiens
DIELTQPPSVSVSPGETASITCSGDKLGDYYVHVWQQKPGQAPVLVIYQDSKRPSGIPERFSGSNSGNTATLTISGTQAEDEADYYCGATDLSPWSIVFGGGTKLTVL

SEQ ID NO: 50 - DNA, 324 bp, Homo sapiens
gatatcgaactgacccagccgccgagcgtgagcgtgagcccgggcgagaccgcgagcattacctgtagcggcgataaactgggtgactactacgttcattggtaccagcagaaaccgggccaggcgccggtgctggtgatctaccaggactctaaacgtccgagcggcatcccggaacgttttagcggatccaacagcggcaacaccgcgaccctgaccattagcggcacccaggcggaagacgaagcggattattactgcggtgctactgacctgtctccgtggtctatcgtgtttggcggcggcacgaagttaaccgtccta

SEQ ID NO: 51 - protein, 214 aa, Homo sapiens
DIELTQPPSVSVSPGETASITCSGDKLGDYYVHWYQQKPGQAPVLVIYQDSKRPSGIPERFSGSNSGNTATLTISGTQAEDEADYYCGATDLSPWSIVFGGGTKLTVLGQPKAAPSVTLFPPSSEELQANKATLVCLISDFYPGAVTVAWKADSSPVKAGVETTTPSKQSNNKYAASSYLSLTPEQWKSMRSYSCQVTHEGSTVEKTVAPTECS

SEQ ID NO: 52 - DNA, 642 bp, Homo sapiens
gatatcgaactgacccagccgccgagcgtgagcgtgagcccgggcgagaccgcgagcattacctgtagcggcgataaactgggtgactactacgttcattggtaccagcagaaaccgggccaggcgccggtgctggtgatctaccaggactctaaacgtccgagcggcatcccggaacgttttagcggatccaacagcggcaacaccgcgaccctgaccattagcggcacccaggcggaagacgaagcggattattactgcggtgctactgacctgtctccgtggtctatcgtgtttggcggcggcacgaagttaaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 53 - protein, 10 aa, Homo sapiens
GGTFSSYAIS

SEQ ID NO: 54 - protein, 17 aa, Homo sapiens
GIIPVLGTANYAQKFQG

SEQ ID NO: 55 - protein, 8 aa, Homo sapiens
VPTDYFDY

SEQ ID NO: 56 - protein, 5 aa, Homo sapiens
SYAIS

SEQ ID NO: 57 - protein, 17 aa, Homo sapiens
GIIPVLGTANYAQKFQG

SEQ ID NO: 58 - protein, 8 aa, Homo sapiens
VPTDYFDY

SEQ ID NO: 59 - protein, 7 aa, Homo sapiens
GGTFSSY

SEQ ID NO: 60 - protein, 6 aa, Homo sapiens
IPVLGT

SEQ ID NO: 61 - protein, 8 aa, Homo sapiens
VPTDYFDY

SEQ ID NO: 62 - protein, 117 aa, Homo sapiens
QVQLVQSGAEVKKPGSSVKVSCKASGGTFSSYAISVWRQAPGQGLEWMGGIIPVLGTANYAQKFQGRVTITADESTSTAYMELSSLRSEDTAVYYCARVPTDYFDYWGQGTLVTVSS

SEQ ID NO: 63 - DNA, 351 bp, Homo sapiens
caggtgcagctggtgcagtcaggcgccgaagtgaagaaacccggctctagcgtgaaagtcagctgtaaagctagtggcggcaccttctctagctacgctattagctgggtcagacaggccccaggtcagggcctggagtggatgggcggaattatccccgtgctgggcaccgctaactacgctcagaaatttcagggtagagtgactatcaccgccgacgagtctactagcaccgcctatatggaactgtctagcctgagatcagaggacaccgccgtctactactgcgctagagtgcctaccgactacttcgactactggggtcagggcaccctggtcaccgtgtctagc

SEQ ID NO: 64 - protein, 447 aa, Homo sapiens
QVQLVQSGAEVKKPGSSVKVSCKASGGTFSSYAISWVRQAPGQGLEWMGGIIPVLGTANYAQKFQGRVTITADESTSTAYMELSSLRSEDTAVYYCARVPTDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNGVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - DNA, 1341 bp, Homo sapiens
caggtgcagctggtgcagtcaggcgccgaagtgaagaaacccggctctagcgtgaaagtcagctgtaaagctagtggcggcaccttctctagctacgctattagctgggtcagacaggccccaggtcagggcctggagtggatgggcggaattatccccgtgctgggcaccgctaactacgctcagaaatttcagggtagagtgactatcaccgccgacgagtctactagcaccgcctatatggaactgtctagcctgagatcagaggacaccgccgtctactactgcgctagagtgcctaccgactacttcgactactggggtcagggcaccctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 66 - protein, 11 aa, Homo sapiens
SGDNLGSKYVH

SEQ ID NO: 67 - protein, 7 aa, Homo sapiens
DDNKRPS

SEQ ID NO: 68 - protein, 9 aa, Homo sapiens
QSWTLGNWV

SEQ ID NO: 69 - protein, 11 aa, Homo sapiens
SGDNLGSKYVH

SEQ ID NO: 70 - protein, 7 aa, Homo sapiens
DDNKRPS

SEQ ID NO: 71 - protein, 9 aa, Homo sapiens
QSWTLGNWV

SEQ ID NO: 72 - protein, 7 aa, Homo sapiens
DNLGSKY

SEQ ID NO: 73 - skipped

SEQ ID NO: 74 - protein, 6 aa, Homo sapiens
WTLGNW

SEQ ID NO: 75 - protein, 106 aa, Homo sapiens
DIELTQPPSVSVSPGQTASITCSGDNLGSKYVHWYGQKPGQAPVLVIYDDNKRPSGIPERFSGSNSGNTATLTISGTQAEDEADYYCQSWTLGNVWFGGGTKLTVL

SEQ ID NO: 76 - DNA, 318 bp, Homo sapiens
gatatcgagctgactcagccccctagcgtcagcgtcagccctggtcagaccgcctctatcacctgtagcggcgataacctgggctctaaatacgtgcactggtatcagcagaagcccggtcaggcccccgtgctggtgatctacgacgataacaagcggcctagcggaatccccgagcggtttagcggctctaatagcggtaacaccgctaccctgactattagcggcactcaggccgaggacgaggccgactactactgtcagtcctggaccctgggcaactgggtgttcggcggaggcactaagctgaccgtgctg

SEQ ID NO: 77 - protein, 212 aa, Homo sapiens
DIELTQPPSVSVSPGQTASITCSGDNLGSKYVHWVQQKPGGAPVLVIYDDNKRPSGIPERFSGSNSGNTATLTISGTQAEDEADYYCGSWTLGNWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 78 - DNA, 636 bp, Homo sapiens
gatatcgagctgactcagccccctagcgtcagcgtcagccctggtcagaccgcctctatcacctgtagcggcgataacctgggctctaaatacgtgcactggtatcagcagaagcccggtcaggcccccgtgctggtgatctacgacgataacaagcggcctagcggaatccccgagcggtttagcggctctaatagcggtaacaccgctaccctgactattagcggcactcaggccgaggacgaggccgactactactgtcagtcctggaccctgggcaactgggtgttcggcggaggcactaagctgaccgtgctgggtcagcctaaggctgcccccagcgtgaccctgttcccccccagcagcgaggagctgcaggccaacaaggccaccctggtgtgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggaaaagaccgtggccccaaccgagtgcagc

SEQ ID NO: 79 - protein, 10 aa, Homo sapiens
GGTFSSYAIS

SEQ ID NO: 80 - protein, 17 aa, Homo sapiens
GIIPVLGTANYAQKFQG

SEQ ID NO: 81 - protein, 8 aa, Homo sapiens
VPTDYFDY

SEQ ID NO: 82 - protein, 5 aa, Homo sapiens
SYAIS

SEQ ID NO: 83 - protein, 17 aa, Homo sapiens
GIIPVLGTANYAQKFQG

SEQ ID NO: 84 - protein, 8 aa, Homo sapiens
VPTDYFDY

SEQ ID NO: 85 - protein, 7 aa, Homo sapiens
GGTFSSY

SEQ ID NO: 86 - protein, 6 aa, Homo sapiens
IPVLGT

SEQ ID NO: 87 - protein, 8 aa, Homo sapiens
VPTDYFDY

SEQ ID NO: 88 - protein, 117 aa, Homo sapiens
QVQLVQSGAEVKKPGSSVKVSCKASGGTFSSYAISWVRQAPGQGLEWMGGIIPVLGTANYAQKFQGRVTITADESTSTAYMELSSLRSEDTAVYYCARVPTDYFDYWGQGTLVTVSS

SEQ ID NO: 89 - DNA, 351 bp, Homo sapiens
caggtgcaattggtgcagagcggtgccgaagtgaaaaaaccgggcagcagcgtgaaagttagctgcaaagcatccggagggacgttttcttcttacgctatctcttgggtgcgccaggccccgggccagggcctcgagtggatgggcggtatcatcccggttctgggcactgcgaactacgcccagaaatttcagggccgggtgaccattaccgccgatgaaagcaccagcaccgcctatatggaactgagcagcctgcgcagcgaagatacggccgtgtattattgcgcgcgtgttccgactgactacttcgattactggggccaaggcaccctggtgactgttagctca

SEQ ID NO: 90 - protein, 446 aa, Homo sapiens
QVQLVQSGAEVKKPGSSVKVSCKASGGTFSSYAISWVRQAPGQGLEWMGGIIPVLGTANYAQKFQGRVTITADESTSTAYMELSSLRSEDTAVYYCARVPTDYFDYWGQGTLVTVSSASTKGPSVFPLAPSSKSTSGGTAALGCLVKDYFPEPVTVSWNSGALTSGVHTFPAVLQSSGLYSLSSWTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - DNA, 1341 bp, Homo sapiens
caggtgcaattggtgcagagcggtgccgaagtgaaaaaaccgggcagcagcgtgaaagttagctgcaaagcatccggagggacgttttcttcttacgctatctcttgggtgcgccaggccccgggccagggcctcgagtggatgggcggtatcatcccggttctgggcactgcgaactacgcccagaaatttcagggccgggtgaccattaccgccgatgaaagcaccagcaccgcctatatggaactgagcagcctgcgcagcgaagatacggccgtgtattattgcgcgcgtgttccgactgactacttcgattactggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 92 - protein, 11 aa, Homo sapiens
SGDNLGSKYVH

SEQ ID NO: 93 - protein, 7 aa, Homo sapiens
DDNKRPS

SEQ ID NO: 94 - protein, 9 aa, Homo sapiens
QSWTLGNWV

SEQ ID NO: 95 - protein, 11 aa, Homo sapiens
SGDNLGSKYVH

SEQ ID NO: 96 - protein, 7 aa, Homo sapiens
DDNKRPS

SEQ ID NO: 97 - protein, 9 aa, Homo sapiens
QSWTLGNWV

SEQ ID NO: 98 - protein, 7 aa, Homo sapiens
DNLGSKY

SEQ ID NO: 99 - skipped

SEQ ID NO: 100 - protein, 6 aa, Homo sapiens
WTLGNW

SEQ ID NO: 101 - protein, 106 aa, Homo sapiens
DIELTQPPSVSVSPGQTASITCSGDNLGSKYVHWYQQKPGQAPVLVIYDDNKRPSGIPERFSGSNSGNTATLTISGTQAEDEADYYCQSWTLGNWVFGGGTKLTVL

SEQ ID NO: 102 - DNA, 318 bp, Homo sapiens
gatatcgaactgacccagccgccgagcgtgagcgtgagcccgggccagaccgcgagcattacctgtagcggcgataacctgggttctaaatacgttcattggtaccagcagaaaccgggccaggcgccggtgctggtgatctacgacgacaacaaacgtccgagcggcatcccggaacgttttagcggatccaacagcggcaacaccgcgaccctgaccattagcggcacccaggcggaagacgaagcggattattactgccagtcttggactctgggtaactgggtgtttggcggcggcacgaagttaaccgtccta

SEQ ID NO: 103 - protein, 212 aa, Homo sapiens
DIELTQPPSVSVSPGQTASITCSGDNLGSKYVHWYQQKPGQAPVLVIYDDNKRPSGIPERFSGSNSGNTATLTISGTQAEDEADYYCQSWTLGNWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 104 - DNA, 636 bp, Homo sapiens
gatatcgaactgacccagccgccgagcgtgagcgtgagcccgggccagaccgcgagcattacctgtagcggcgataacctgggttctaaatacgttcattggtaccagcagaaaccgggccaggcgccggtgctggtgatctacgacgacaacaaacgtccgagcggcatcccggaacgttttagcggatccaacagcggcaacaccgcgaccctgaccattagcggcacccaggcggaagacgaagcggattattactgccagtcttggactctgggtaactgggtgtttggcggcggcacgaagttaaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 105 - protein, 10 aa, Homo sapiens
GGTFSDNAIS

SEQ ID NO: 106 - protein, 17 aa, Homo sapiens
GINPDFGWANYAQKFQG

SEQ ID NO: 107 - protein, 7 aa, Homo sapiens
DSSGMGY

SEQ ID NO: 108 - protein, 5 aa, Homo sapiens
DNAIS

SEQ ID NO: 109 - protein, 17 aa, Homo sapiens
GINPDFGWANYAQKFQG

SEQ ID NO: 110 - protein, 7 aa, Homo sapiens
DSSGMGY

SEQ ID NO: 111 - protein, 7 aa, Homo sapiens
GGTFSDN

SEQ ID NO: 112 - protein, 6 aa, Homo sapiens
NPDFGW

SEQ ID NO: 113 - protein, 7 aa, Homo sapiens
DSSGMGY

SEQ ID NO: 114 - protein, 116 aa, Homo sapiens
QVQLVQSGAEVKKPGSSVKVSCKASGGTFSDNAISWVRQAPGQGLEWMGGINPDFGWANYAQKFQGRVTITADESTSTAYMELSSLRSEDTAVYYCARDSSGMGYWGQGTLVTVSS

SEQ ID NO: 115 - DNA, 348 bp, Homo sapiens
caggtgcagctggtgcagtcaggcgccgaagtgaagaaacccggctctagcgtgaaagtcagctgtaaagctagtggcggcacctttagcgataacgctattagctgggtcagacaggccccaggtcagggcctggagtggatgggcgggattaaccccgacttcggctgggctaactacgctcagaaatttcagggtagagtgactatcaccgccgacgagtctactagcaccgcctatatggaactgtctagcctgagatcagaggacaccgccgtctactactgcgctagggactctagcggaatgggctactggggtcagggcaccctggtcaccgtgtctagc

SEQ ID NO: 116 - protein, 446 aa, Homo sapiens
QVQLVQSGAEVKKPGSSVKVSCKASGGTFSDNAISVWRQAPGQGLEWMGGINPDFGWANYAQKFGGRVTITADESTSTAYMELSSLRSEDTAVYYCARDSSGMGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - DNA, 1338 bp, Homo sapiens
caggtgcagctggtgcagtcaggcgccgaagtgaagaaacccggctctagcgtgaaagtcagctgtaaagctagtggcggcacctttagcgataacgctattagctgggtcagacaggccccaggtcagggcctggagtggatgggcgggattaaccccgacttcggctgggctaactacgctcagaaatttcagggtagagtgactatcaccgccgacgagtctactagcaccgcctatatggaactgtctagcctgagatcagaggacaccgccgtctactactgcgctagggactctagcggaatgggctactggggtcagggcaccctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 118 - protein, 11 aa, Homo sapiens
RASQDISSYLN

SEQ ID NO: 119 - protein, 7 aa, Homo sapiens
DASTLQS

SEQ ID NO: 120 - protein, 10 aa, Homo sapiens
QQSGHWLSKT

SEQ ID NO: 121 - protein, 11 aa, Homo sapiens
RASQDISSYLN

SEQ ID NO: 122 - protein, 7 aa, Homo sapiens
DASTLQS

SEQ ID NO: 123 - protein, 10 aa, Homo sapiens
QQSGHWLSKT

SEQ ID NO: 124 - protein, 7 aa, Homo sapiens
SQDISSY

SEQ ID NO: 125 - skipped

SEQ ID NO: 126 - protein, 7 aa, Homo sapiens
SGHWLSK

SEQ ID NO: 127 - protein, 108 aa, Homo sapiens
DIQMTGSPSSLSASVGDRVTITCRASQDISSYLNWYQQKPGKAPKLLIYDASTLQSGVPSRFSGSGSGTDFTLTISSLQPEDFATYYCQQSGHWLSKTFGQGTKVEIK

SEQ ID NO: 128 - DNA, 324 bp, Homo sapiens
gatattcagatgactcagtcacctagtagcctgagcgctagtgtgggcgatagagtgactatcacctgtagagcctctcaggatatctctagctacctgaactggtatcagcagaagcccggtaaagcccctaagctgctgatctacgacgcctctaccctgcagtcaggcgtgccctctaggtttagcggtagcggtagtggcaccgacttcaccctgactatctctagcctgcagcccgaggacttcgctacctactactgtcagcagtcaggccactggctgtctaagaccttcggtcagggcactaaggtcgagattaag

SEQ ID NO: 129 - protein, 215 aa, Homo sapiens
DIQMTQSPSSLSASVGDRVTITCRASQDISSYLNWYQQKPGKAPKLLIYDASTLQSGVPSRFSGSGSGTDFTLTISSLQPEDFATYYCQQSGHWLSKTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 130 - DNA, 645 bp, Homo sapiens
gatattcagatgactcagtcacctagtagcctgagcgctagtgtgggcgatagagtgactatcacctgtagagcctctcaggatatctctagctacctgaactggtatcagcagaagcccggtaaagcccctaagctgctgatctacgacgcctctaccctgcagtcaggcgtgccctctaggtttagcggtagcggtagtggcaccgacttcaccctgactatctctagcctgcagcccgaggacttcgctacctactactgtcagcagtcaggccactggctgtctaagaccttcggtcagggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 131 - protein, 10 aa, Homo sapiens
GGTFGDNAIS

SEQ ID NO: 132 - protein, 17 aa, Homo sapiens
GINPDFGWANYAQKFQG

SEQ ID NO: 133 - protein, 7 aa, Homo sapiens
DSSGMGY

SEQ ID NO: 134 - protein, 5 aa, Homo sapiens
DNAIS

SEQ ID NO: 135 - protein, 17 aa, Homo sapiens
GINPDFGWANYAQKFQG

SEQ ID NO: 136 - protein, 7 aa, Homo sapiens
DSSGMGY

SEQ ID NO: 137 - protein, 7 aa, Homo sapiens
GGTFSDN

SEQ ID NO: 138 - protein, 6 aa, Homo sapiens
NPDFGW

SEQ ID NO: 139 - protein, 7 aa, Homo sapiens
DSSGMGY

SEQ ID NO: 140 - protein, 116 aa, Homo sapiens
QVQLVQSGAEVKKPGSSVKVSCKASGGTFSDNAISVWRQAPGQGLEWMGGINPDFGWANYAQKFQGRVTITADESTSTAYMELSSLRSEDTAVYYCARDSSGMGYWGQGTLVTVSS

SEQ ID NO: 141 - DNA, 348 bp, Homo sapiens
caggtgcaattggtgcagagcggtgccgaagtgaaaaaaccgggcagcagcgtgaaagttagctgcaaagcatccggagggacgttttctgacaacgctatctcttgggtgcgccaggccccgggccagggcctcgagtggatgggcggtatcaacccggacttcggctgggcgaactacgcccagaaatttcagggccgggtgaccattaccgccgatgaaagcaccagcaccgcctatatggaactgagcagcctgcgcagcgaagatacggccgtgtattattgcgcgcgtgactcttctggtatgggttactggggccaaggcaccctggtgactgttagctca

SEQ ID NO: 142 - protein, 446 aa, Homo sapiens
QVQLVQSGAEVKKPGSSVKVSCKASGGTFSDNAISWVRQAPGQGLEWMGGINPDFGWANYAQKFQGRVTITADESTSTAYMELSSLRSEDTAVYYCARDSSGMGYWGQGTLVTVSSASTKGPSVFPLAPSSKSTSGGTAALGCLVKDYFPEPVTVSWNSGALTSGVHTFPAVLQSSGLYSLSSVVTVPSSSLGTGTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPPEPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - DNA, 1338 bp, Homo sapiens
caggtgcaattggtgcagagcggtgccgaagtgaaaaaaccgggcagcagcgtgaaagttagctgcaaagcatccggagggacgttttctgacaacgctatctcttgggtgcgccaggccccgggccagggcctcgagtggatgggcggtatcaacccggacttcggctgggcgaactacgcccagaaatttcagggccgggtgaccattaccgccgatgaaagcaccagcaccgcctatatggaactgagcagcctgcgcagcgaagatacggccgtgtattattgcgcgcgtgactcttctggtatgggttactggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 144 - protein, 11 aa, Homo sapiens
RASQDISSYLN

SEQ ID NO: 145 - protein, 7 aa, Homo sapiens
DASTLQS

SEQ ID NO: 146 - protein, 10 aa, Homo sapiens
QQSGHWLSKT

SEQ ID NO: 147 - protein, 11 aa, Homo sapiens
RASQDISSYLN

SEQ ID NO: 148 - protein, 7 aa, Homo sapiens
DASTLQS

SEQ ID NO: 149 - protein, 10 aa, Homo sapiens
QQSGHWLSKT

SEQ ID NO: 150 - protein, 7 aa, Homo sapiens
SQDISSY

SEQ ID NO: 151 - skipped

SEQ ID NO: 152 - protein, 7 aa, Homo sapiens
SGHWLSK

SEQ ID NO: 153 - protein, 108 aa, Homo sapiens
DIQMTQSPSSLSASVGDRVTITCRASQDISSYLNWYQQKPGKAPKLLIYDASTLQSGVPSRFSGSGSGTDFTLTISSLQPEDFATYYCQQSGHWLSKTFGQGTKVEIK

SEQ ID NO: 154 - DNA, 324 bp, Homo sapiens
gatatccagatgacccagagcccgagcagcctgagcgccagcgtgggcgatcgcgtgaccattacctgcagagccagccaggacatttcttcttacctgaactggtaccagcagaaaccgggcaaagcgccgaaactattaatctacgacgcttctactctgcaaagcggcgtgccgagccgctttagcggcagcggatccggcaccgatttcaccctgaccattagctctctgcaaccggaagactttgcgacctattattgccagcagtctggtcattggctgtctaaaacctttggccagggcacgaaagttgaaattaaa

SEQ ID NO: 155 - protein, 215 aa, Homo sapiens; identical to SEQ ID NO: 129

SEQ ID NO: 156 - DNA, 645 bp, Homo sapiens
gatatccagatgacccagagcccgagcagcctgagcgccagcgtgggcgatcgcgtgaccattacctgcagagccagccaggacatttcttcttacctgaactggtaccagcagaaaccgggcaaagcgccgaaactattaatctacgacgcttctactctgcaaagcggcgtgccgagccgctttagcggcagcggatccggcaccgatttcaccctgaccattagctctctgcaaccggaagactttgcgacctattattgccagcagtctggtcattggctgtctaaaacctttggccagggcacgaaagttgaaattaaacgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaaccggggcgagtgt

SEQ ID NO: 157 - protein, 10 aa, Homo sapiens
GGTFRDYAIS

SEQ ID NO: 158 - protein, 17 aa, Homo sapiens
GIIPAFGTANYAQKFQG

SEQ ID NO: 159 - protein, 18 aa, Homo sapiens
EQDPEYGYGGYPYEAMDV

SEQ ID NO: 160 - protein, 5 aa, Homo sapiens
DYAIS

SEQ ID NO: 161 - protein, 17 aa, Homo sapiens
GIIPAFGTANYAQKFQG

SEQ ID NO: 162 - protein, 18 aa, Homo sapiens
EQDPEYGYGGYPYEAMDV

SEQ ID NO: 163 - protein, 7 aa, Homo sapiens
GGTFRDY

SEQ ID NO: 164 - protein, 6 aa, Homo sapiens
IPAFGT

SEQ ID NO: 165 - protein, 18 aa, Homo sapiens
EQDPEYGYGGYPYEAMDV

SEQ ID NO: 166 - protein, 127 aa, Homo sapiens
QVQLVQSGAEVKKPGSSVKVSCKASGGTFRDYAISVWRQAPGQGLEWMGGIIPAFGTANYAQKFQGRVTITADESTSTAYMELSSLRSEDTAVYYCAREQDPEYGYGGYPYEAMDVWGQGTLVTVSS

SEQ ID NO: 167 - DNA, 381 bp, Homo sapiens
caggtgcagctggtgcagtcaggcgccgaagtgaagaaacccggctctagcgtgaaagtcagctgtaaagctagtggcggcacctttagagactacgctattagctgggtcagacaggccccaggtcagggcctggagtggatgggcggaattatccccgccttcggcaccgctaactacgctcagaaatttcagggtagagtgactatcaccgccgacgagtctactagcaccgcctatatggaactgtctagcctgagatcagaggacaccgccgtctactactgcgctagagagcaggaccccgagtacggctacggcggctacccctacgaggctatggacgtgtggggtcagggcaccctggtcaccgtgtctagc

SEQ ID NO: 168 - protein, 457 aa, Homo sapiens
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPEYGYGGYPYEAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 169 - protein, 1371 aa, Homo sapiens
CAGGTGCAGCTGGTGCAGTCAGGCGCCGAAGTGAAGAAACCCGGCTCTAGCGTGAAAGTCAGCTGTAAAGCTAGTGGCGGCACCTTTAGAGACTACGCTATTAGCTGGGTCAGACAGGCCCCAGGTCAGGGCCTGGAGTGGATGGGCGGAATTATCCCCGCCTTCGGCACCGCTAACTACGCTCAGAAATTTCAGGGTAGAGTGACTATCACCGCCGACGAGTCTACTAGCACCGCCTATATGGAACTGTCTAGCCTGAGATCAGAGGACACCGCCGTCTACTACTGCGCTAGAGAGCAGGACCCCGAGTACGGCTACGGCGGCTACCCCTACGAGGCTATGGACGTGTGGGGTCAGGGCACCCTGGTCACCGTGTCTAGCGCTAGCACTAAGGGCCCAAGTGTGTTTCCCCTGGCCCCCAGCAGCAAGTCTACTTCCGGCGGAACTGCTGCCCTGGGTTGCCTGGTGAAGGACTACTTCCCCGAGCCCGTGACAGTGTCCTGGAACTCTGGGGCTCTGACTTCCGGCGTGCACACCTTCCCCGCCGTGCTGCAGAGCAGCGGCCTGTACAGCCTGAGCAGCGTGGTGACAGTGCCCTCCAGCTCTCTGGGAACCCAGACCTATATCTGCAACGTGAACCACAAGCCCAGCAACACCAAGGTGGACAAGAGAGTGGAGCCCAAGAGCTGCGACAAGACCCACACCTGCCCCCCCTGCCCAGCTCCAGAACTGCTGGGAGGGCCTTCCGTGTTCCTGTTCCCCCCCAAGCCCAAGGACACCCTGATGATCAGCAGGACCCCCGAGGTGACCTGCGTGGTGGTGGACGTGTCCCACGAGGACCCAGAGGTGAAGTTCAACTGGTACGTGGACGGCGTGGAGGTGCACAACGCCAAGACCAAGCCCAGAGAGGAGCAGTACAACAGCACCTACAGGGTGGTGTCCGTGCTGACCGTGCTGCACCAGGACTGGCTGAACGGCAAAGAATACAAGTGCAAAGTCTCCAACAAGGCCCTGCCAGCCCCAATCGAAAAGACAATCAGCAAGGCCAAGGGCCAGCCACGGGAGCCCCAGGTGTACACCCTGCCCCCCAGCCGGGAGGAGATGACCAAGAACCAGGTGTCCCTGACCTGTCTGGTGAAGGGCTTCTACCCCAGCGATATCGCCGTGGAGTGGGAGAGCAACGGCCAGCCCGAGAACAACTACAAGACCACCCCCCCAGTGCTGGACAGCGACGGCAGCTTCTTCCTGTACAGCAAGCTGACCGTGGACAAGTCCAGGTGGCAGCAGGGCAACGTGTTCAGCTGCAGCGTGATGCACGAGGCCCTGCACAACCACTACACCCAGAAGTCCCTGAGCCTGAGCCCCGGCAAG

SEQ ID NO: 170 - protein, 11 aa, Homo sapiens
SGDNIPQHSVH

SEQ ID NO: 171 - protein, 7 aa, Homo sapiens
DDTERPS

SEQ ID NO: 172 - protein, 11 aa, Homo sapiens
SSWDSSMDSVV

SEQ ID NO: 173 - protein, 11 aa, Homo sapiens
SGDNIPQHSVH

SEQ ID NO: 174 - protein, 7 aa, Homo sapiens
DDTERPS

SEQ ID NO: 175 - protein, 11 aa, Homo sapiens
SSWDSSMDSVV

SEQ ID NO: 176 - protein, 7 aa, Homo sapiens
DNIPQHS

SEQ ID NO: 177 - skipped

SEQ ID NO: 178 - protein, 8 aa, Homo sapiens
WDSSMDSV

SEQ ID NO: 179 - protein, 107 aa, Homo sapiens
SYELTQPLSVSVALGQTARITCSGDNIPQHSVHWYQQKPGQAPVLVIYDDTERPSGIPERFSGSNSGNTATLTISRAQAGDEADYYCSSWDSSMDSWFGGGTKLTVL

SEQ ID NO: 180 - DNA, 324 bp, Homo sapiens
agctacgagctgactcagcccctgagcgtcagcgtggccctgggtcagaccgctagaatcacctgtagcggcgataatatccctcagcactcagtgcactggtatcagcagaagcccggtcaggcccccgtgctggtgatctacgacgacaccgagcggcctagcggaatccccgagcggtttagcggctctaatagcggtaacaccgctaccctgactatctctagggctcaggccggcgacgaggccgactactactgctctagctgggatagctctatggatagcgtggtgttcggcggaggcactaagctgaccgtgctg

SEQ ID NO: 181 - protein, 214 aa, Homo sapiens
SYELTQPLSVSVALGQTARITCSGDNIPQHSVHWYQQKPGQAPVLVIYDDTERPSGIPERFSGSNSGNTATLTISRAQAGDEADYYCSSWDSSMDSVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 182 - DNA, 642 bp, Homo sapiens
agctacgagctgactcagcccctgagcgtcagcgtggccctgggtcagaccgctagaatcacctgtagcggcgataatatccctcagcactcagtgcactggtatcagcagaagcccggtcaggcccccgtgctggtgatctacgacgacaccgagcggcctagcggaatccccgagcggtttagcggctctaatagcggtaacaccgctaccctgactatctctagggctcaggccggcgacgaggccgactactactgctctagctgggatagctctatggatagcgtggtgttcggcggaggcactaagctgaccgtgctgggtcagcctaaggctgcccccagcgtgaccctgttcccccccagcagcgaggagctgcaggccaacaaggccaccctggtgtgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggaaaagaccgtggccccaaccgagtgcagc

SEQ ID NO: 183 - protein, 10 aa, Homo sapiens
GGTFRDYAIS

SEQ ID NO: 184 - protein, 17 aa, Homo sapiens
GIIPAFGTANYAQKFQG

SEQ ID NO: 185 - protein, 18 aa, Homo sapiens
EQDPEYGYGGYPYEAMDV

SEQ ID NO: 186 - protein, 5 aa, Homo sapiens
DYAIS

SEQ ID NO: 187 - protein, 17 aa, Homo sapiens
GIIPAFGTANYAQKFQG

SEQ ID NO: 188 - protein, 18 aa, Homo sapiens
EQDPEYGYGGYPYEAMDV

SEQ ID NO: 189 - protein, 7 aa, Homo sapiens
GGTFRDY

SEQ ID NO: 190 - protein, 6 aa, Homo sapiens
IPAFGT

SEQ ID NO: 191 - protein, 18 aa, Homo sapiens
EQDPEYGYGGYPYEAMDV

SEQ ID NO: 192 - protein, 127 aa, Homo sapiens
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPEYGYGGYPYEAMDVWGQGTLVTVSS

SEQ ID NO: 193 - DNA, 381 bp, Homo sapiens
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacaggacccggaatacggttacggtggttacccgtatgaagctatggatgtttggggccaaggcaccctggtgactgttagctca

SEQ ID NO: 194 - protein, 457 aa, Homo sapiens
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPEYGYGGYPYEAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 195 - DNA, 1371 bp, Homo sapiens
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacaggacccggaatacggttacggtggttacccgtatgaagctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 196 - protein, 11 aa, Homo sapiens
SGDNIPQHSVH

SEQ ID NO: 197 - protein, 7 aa, Homo sapiens
DDTERPS

SEQ ID NO: 198 - protein, 11 aa, Homo sapiens
SSWDSSMDSVV

SEQ ID NO: 199 - protein, 11 aa, Homo sapiens
SGDNIPQHSVH

SEQ ID NO: 200 - protein, 7 aa, Homo sapiens
DDTERPS

SEQ ID NO: 201 - protein, 11 aa, Homo sapiens
SSWDSSMDSVV

SEQ ID NO: 202 - protein, 7 aa, Homo sapiens
DNIPQHS

SEQ ID NO: 203 - skipped

SEQ ID NO: 204 - protein, 8 aa, Homo sapiens
WDSSMDSV

SEQ ID NO: 205 - protein, 107 aa, Homo sapiens
SYELTQPLSVSVALGQTARITCSGDNIPQHSVHWYQQKPGQAPVLVIYDDTERPSGIPERFSGSNSGNTATLTISRAQAGDEADYYCSSWDSSMPSWFGGGTKLTVL

SEQ ID NO: 206 - DNA, 324 bp, Homo sapiens
agctacgaactgacccagccgctgagcgtgagcgtggccctgggccagaccgcgaggattacctgtagcggcgataacatcccgcagcattctgttcattggtaccagcagaaaccgggccaggcgccggtgctggtgatctacgacgacactgaacgtccgagcggcatcccggaacgttttagcggatccaacagcggcaacaccgcgaccctgaccattagcagggcccaggcgggcgacgaagcggattattactgctcttcttgggactcttctatggactctgttgtgtttggcggcggcacgaagttaaccgtccta

SEQ ID NO: 207 - protein, 214 aa, Homo sapiens; identical to SEQ ID NO: 181

SEQ ID NO: 208 - DNA, 642 bp, Homo sapiens
agctacgaactgacccagccgctgagcgtgagcgtggccctgggccagaccgcgaggattacctgtagcggcgataacatcccgcagcattctgttcattggtaccagcagaaaccgggccaggcgccggtgctggtgatctacgacgacactgaacgtccgagcggcatcccggaacgttttagcggatccaacagcggcaacaccgcgaccctgaccattagcagggcccaggcgggcgacgaagcggattattactgctcttcttgggactcttctatggactctgttgtgtttggcggcggcacgaagttaaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 209 - protein, 10 aa, Homo sapiens
GFTFPTHGLH

SEQ ID NO: 210 - protein, 17 aa, Homo sapiens
AISYDASETNYADSVKG

SEQ ID NO: 211 - protein, 9 aa, Homo sapiens
ESIGGYFDY

SEQ ID NO: 212 - protein, 5 aa, Homo sapiens
THGLH

SEQ ID NO: 213 - protein, 17 aa, Homo sapiens
AISYDASETNYADSVKG

SEQ ID NO: 214 - protein, 9 aa, Homo sapiens
ESIGGYFDY

SEQ ID NO: 215 - protein, 7 aa, Homo sapiens
GFTFPTH

SEQ ID NO: 216 - protein, 6 aa, Homo sapiens
SYDASE

SEQ ID NO: 217 - protein, 9 aa, Homo sapiens
ESIGGYFDY

SEQ ID NO: 218 - protein, 118 aa, Homo sapiens
QVQLLESGGGLVQPGGSLRLSCAASGFTFPTHGLHVWRQAPGKGLEVWSAISYDASETNYADSVKGRFTISRDNSKNTLYLQMNSLRAEDTAVYYCARESIGGYFDYWGQGTLVTVSS

SEQ ID NO: 219 - DNA, 354 bp, Homo sapiens
caggtgcagctgctggaatcaggcggcggactggtgcagcctggcggtagcctgagactgagctgcgctgctagtggcttcaccttccctactcacggcctgcactgggtcagacaggcccctggtaaaggcctggagtgggtcagcgctattagctacgacgctagtgaaactaactacgccgatagcgtgaagggccggttcactatctctagggataactctaagaacaccctgtacctgcagatgaatagcctgagagccgaggacaccgccgtctactactgcgctagagagtctatcggcggctacttcgactactggggtcagggcaccctggtcaccgtgtctagc

SEQ ID NO: 220 - protein, 448 aa, Homo sapiens
QVQLLESGGGLVQPGGSLRLSCAASGFTFPTHGLHWVRQAPGKGLEWVSAISYDASETNYADSVKGRFTISRDNSKNTLYLQMNSLRAEDTAVYYCARESIGGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 221 - protein, 1344 aa, Homo sapiens
CAGGTGCAGCTGCTGGAATCAGGCGGCGGACTGGTGCAGCCTGGCGGTAGCCTGAGACTGAGCTGCGCTGCTAGTGGCTTCACCTTCCCTACTCACGGCCTGCACTGGGTCAGACAGGCCCCTGGTAAAGGCCTGGAGTGGGTCAGCGCTATTAGCTACGACGCTAGTGAAACTAACTACGCCGATAGCGTGAAGGGCCGGTTCACTATCTCTAGGGATAACTCTAAGAACACCCTGTACCTGCAGATGAATAGCCTGAGAGCCGAGGACACCGCCGTCTACTACTGCGCTAGAGAGTCTATCGGCGGCTACTTCGACTACTGGGGTCAGGGCACCCTGGTCACCGTGTCTAGCGCTAGCACTAAGGGCCCAAGTGTGTTTCCCCTGGCCCCCAGCAGCAAGTCTACTTCCGGCGGAACTGCTGCCCTGGGTTGCCTGGTGAAGGACTACTTCCCCGAGCCCGTGACAGTGTCCTGGAACTCTGGGGCTCTGACTTCCGGCGTGCACACCTTCCCCGCCGTGCTGCAGAGCAGCGGCCTGTACAGCCTGAGCAGCGTGGTGACAGTGCCCTCCAGCTCTCTGGGAACCCAGACCTATATCTGCAACGTGAACCACAAGCCCAGCAACACCAAGGTGGACAAGAGAGTGGAGCCCAAGAGCTGCGACAAGACCCACACCTGCCCCCCCTGCCCAGCTCCAGAACTGCTGGGAGGGCCTTCCGTGTTCCTGTTCCCCCCCAAGCCCAAGGACACCCTGATGATCAGCAGGACCCCCGAGGTGACCTGCGTGGTGGTGGACGTGTCCCACGAGGACCCAGAGGTGAAGTTCAACTGGTACGTGGACGGCGTGGAGGTGCACAACGCCAAGACCAAGCCCAGAGAGGAGCAGTACAACAGCACCTACAGGGTGGTGTCCGTGCTGACCGTGCTGCACCAGGACTGGCTGAACGGCAAAGAATACAAGTGCAAAGTCTCCAACAAGGCCCTGCCAGCCCCAATCGAAAAGACAATCAGCAAGGCCAAGGGCCAGCCACGGGAGCCCCAGGTGTACACCCTGCCCCCCAGCCGGGAGGAGATGACCAAGAACCAGGTGTCCCTGACCTGTCTGGTGAAGGGCTTCTACCCCAGCGATATCGCCGTGGAGTGGGAGAGCAACGGCCAGCCCGAGAACAACTACAAGACCACCCCCCCAGTGCTGGACAGCGACGGCAGCTTCTTCCTGTACAGCAAGCTGACCGTGGACAAGTCCAGGTGGCAGCAGGGCAACGTGTTCAGCTGCAGCGTGATGCACGAGGCCCTGCACAACCACTACACCCAGAAGTCCCTGAGCCTGAGCCCCGGCAAG

SEQ ID NO: 222 - protein, 11 aa, Homo sapiens
SGDALGKNTVS

SEQ ID NO: 223 - protein, 7 aa, Homo sapiens
DDTDRPS

SEQ ID NO: 224 - protein, 9 aa, Homo sapiens
SSTDLSTVV

SEQ ID NO: 225 - protein, 11 aa, Homo sapiens
SGDALGKNTVS

SEQ ID NO: 226 - protein, 7 aa, Homo sapiens
DDTDRPS

SEQ ID NO: 227 - protein, 9 aa, Homo sapiens
SSTDLSTVV

SEQ ID NO: 228 - protein, 7 aa, Homo sapiens
DALGKNT

SEQ ID NO: 229 - skipped

SEQ ID NO: 230 - protein, 6 aa, Homo sapiens
TDLSTV

SEQ ID NO: 231 - protein, 106 aa, Homo sapiens
SYELTQPLSVSVALGQTARITCSGDALGKNTVSWYQQKPGQAPVLVIYDDTDRPSGIPERFSGSNSGNTATLTISRAQAGDEADYYCSSTDLSTVVFGGGTKLTVL

SEQ ID NO: 232 - DNA, 318 bp, Homo sapiens
agctacgagctgactcagcccctgagcgtcagcgtggccctgggtcagaccgctagaatcacctgtagcggcgacgccctgggtaaaaacaccgtcagctggtatcagcagaagcccggtcaggcccccgtgctggtgatctacgacgacaccgatagacctagcggaatccccgagcggtttagcggctctaatagcggtaacaccgctaccctgactatctctagggctcaggccggcgacgaggccgactactactgctctagcaccgacctgagcaccgtggtgttcggcggaggcactaagctgaccgtgctg

SEQ ID NO: 233 - protein, 212 aa, Homo sapiens
SYELTQPLSVSVALGQTARITCSGDALGKNTVSWYQQKPGQAPVLVIYDDTDRPSGIPERFSGSNSGNTATLTISRAQAGDEADYYCSSTDLSTVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 234 - DNA, 636 bp, Homo sapiens
agctacgagctgactcagcccctgagcgtcagcgtggccctgggtcagaccgctagaatcacctgtagcggcgacgccctgggtaaaaacaccgtcagctggtatcagcagaagcccggtcaggcccccgtgctggtgatctacgacgacaccgatagacctagcggaatccccgagcggtttagcggctctaatagcggtaacaccgctaccctgactatctctagggctcaggccggcgacgaggccgactactactgctctagcaccgacctgagcaccgtggtgttcggcggaggcactaagctgaccgtgctgggtcagcctaaggctgcccccagcgtgaccctgttcccccccagcagcgaggagctgcaggccaacaaggccaccctggtgtgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggaaaagaccgtggccccaaccgagtgcagc

SEQ ID NO: 235 - protein, 10 aa, Homo sapiens
GFTFPTHGLH

SEQ ID NO: 236 - protein, 17 aa, Homo sapiens
AISYDASETNYADSVKG

SEQ ID NO: 237 - protein, 9 aa, Homo sapiens
ESIGGYFDY

SEQ ID NO: 238 - protein, 5 aa, Homo sapiens
THGLH

SEQ ID NO: 239 - protein, 17 aa, Homo sapiens
AISYDASETNYADSVKG

SEQ ID NO: 240 - protein, 9 aa, Homo sapiens
ESIGGYFDY

SEQ ID NO: 241 - protein, 7 aa, Homo sapiens
GFTFPTH

SEQ ID NO: 242 - protein, 6 aa, Homo sapiens
SYDASE

SEQ ID NO: 243 - protein, 9 aa, Homo sapiens
ESIGGYFDY

SEQ ID NO: 244 - protein, 118 aa, Homo sapiens
QVQLLESGGGLVQPGGSLRLSCAASGFTFPTHGLHVWRQAPGKGLEWVSAISYDASETNYADSVKGRFTISRDNSKNTLYLQMNSLRAEDTAVYYCARESIGGYFDYWGQGTLVTVSS

SEQ ID NO: 245 - DNA, 354 bp, Homo sapiens
caggtgcaattgctggaaagcggcggtggcctggtgcagccgggtggcagcctgcgtctgagctgcgcggcgtccggattcacctttcctactcatggtctgcattgggtgcgccaggccccgggcaaaggtctcgagtgggtttccgctatctcttacgacgcctctgaaaccaactatgcggatagcgtgaaaggccgctttaccatcagccgcgataattcgaaaaacaccctgtatctgcaaatgaacagcctgcgtgcggaagatacggccgtgtattattgcgcgcgtgaatctatcggtggttacttcgattactggggccaaggcaccctggtgactgttagctca

SEQ ID NO: 246 - protein, 448 aa, Homo sapiens
QVQLLESGGGLVQPGGSLRLSCAASGFTFPTHGLHVWRQAPGKGLEWVSAISYDASETNYADSVKGRFTISRDNSKNTLYLQMNSLRAEDTAVYYCARESIGGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 247 - DNA, 1344 bp, Homo sapiens
caggtgcaattgctggaaagcggcggtggcctggtgcagccgggtggcagcctgcgtctgagctgcgcggcgtccggattcacctttcctactcatggtctgcattgggtgcgccaggccccgggcaaaggtctcgagtgggtttccgctatctcttacgacgcctctgaaaccaactatgcggatagcgtgaaaggccgctttaccatcagccgcgataattcgaaaaacaccctgtatctgcaaatgaacagcctgcgtgcggaagatacggccgtgtattattgcgcgcgtgaatctatcggtggttacttcgattactggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 248 - protein, 11 aa, Homo sapiens
SGDALGKNTVS

SEQ ID NO: 249 - protein, 7 aa, Homo sapiens
DDTDRPS

SEQ ID NO: 250 - protein, 9 aa, Homo sapiens
SSTDLSTVV

SEQ ID NO: 251 - protein, 11 aa, Homo sapiens
SGDALGKNTVS

SEQ ID NO: 252 - protein, 7 aa, Homo sapiens
DDTDRPS

SEQ ID NO: 253 - protein, 9 aa, Homo sapiens
SSTDLSTVV

SEQ ID NO: 254 - protein, 7 aa, Homo sapiens
DALGKNT

SEQ ID NO: 255 - skipped

SEQ ID NO: 256 - protein, 6 aa, Homo sapiens
TDLSTV

SEQ ID NO: 257 - protein, 106 aa, Homo sapiens
SYELTQPLSVSVALGQTARITCSGDALGKNTVSWYQQKPGQAPVLVIYQDTDRPSGIPERFSGSNSGNTATLTISRAQAGDEADYYCSSTDLSTVVFGGGTKLTVL

SEQ ID NO: 258 - DNA, 318 bp, Homo sapiens
agctacgaactgacccagccgctgagcgtgagcgtggccctgggccagaccgcgaggattacctgtagcggcgatgctctgggtaaaaacactgtttcttggtaccagcagaaaccgggccaggcgccggtgctggtgatctacgacgacactgaccgtccgagcggcatcccggaacgttttagcggatccaacagcggcaacaccgcgaccctgaccattagcagggcccaggcgggcgacgaagcggattattactgctcttctactgacctgtctactgttgtgtttggcggcggcacgaagttaaccgtccta

SEQ ID NO: 259 - protein, 211 aa, Homo sapiens
SYELTQPLSVSVALGQTARITCSGDALGKNTVSWYQQKPGQAPVLVIYDDTDRPSGIPERFSGSNSGNTATLTISRAQAGDEADYYCSSTDLSTWFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 260 - DNA, 636 bp, Homo sapiens
agctacgaactgacccagccgctgagcgtgagcgtggccctgggccagaccgcgaggattacctgtagcggcgatgctctgggtaaaaacactgtttcttggtaccagcagaaaccgggccaggcgccggtgctggtgatctacgacgacactgaccgtccgagcggcatcccggaacgttttagcggatccaacagcggcaacaccgcgaccctgaccattagcagggcccaggcgggcgacgaagcggattattactgctcttctactgacctgtctactgttgtgtttggcggcggcacgaagttaaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 261 - protein, 10 aa, Homo sapiens
GFTFPTHGLH

SEQ ID NO: 262 - protein, 17 aa, Homo sapiens
AISYEGSETNYADSVKG

SEQ ID NO: 263 - protein, 9 aa, Homo sapiens
ESIGGYFDY

SEQ ID NO: 264 - protein, 5 aa, Homo sapiens
THGLH

SEQ ID NO: 265 - protein, 17 aa, Homo sapiens
AISYEGSETNYADSVKG

SEQ ID NO: 266 - protein, 9 aa, Homo sapiens
ESIGGYFDY

SEQ ID NO: 267 - protein, 7 aa, Homo sapiens
GFTFPTH

SEQ ID NO: 268 - protein, 6 aa, Homo sapiens
SYEGSE

SEQ ID NO: 269 - protein, 9 aa, Homo sapiens
ESIGGYFDY

SEQ ID NO: 270 - protein, 118 aa, Homo sapiens
QVQLLESGGGLVQPGGSLRLSCAASGFTFPTHGLHVWRQAPGKGLEWVSAISYEGSETNYADSVKGRFTISRDNSKNTLYLQMNSLRAEDTAVYYCARESIGGYFDYWGQGTLVTVSS

SEQ ID NO: 271 - DNA, 354 bp, Homo sapiens
caggtgcagctgctggaatcaggcggcggactggtgcagcctggcggtagcctgagactgagctgcgctgctagtggcttcaccttccctactcacggcctgcactgggtcagacaggcccctggtaaaggcctggagtgggtcagcgctattagctacgagggtagcgagactaactacgccgatagcgtgaagggccggttcactatctctagggataactctaagaacaccctgtacctgcagatgaatagcctgagagccgaggacaccgccgtctactactgcgctagagagtctatcggcggctacttcgactactggggtcagggcaccctggtcaccgtgtctagc

SEQ ID NO: 272 - protein, 448 aa, Homo sapiens
QVGLLESGGGLVQPGGSLRLSCAASGFTFPTHGLHVWRQAPGKGLEVWSAISYEGSETNYADSVKGRFTISRDNSKNTLYLQMNSLRAEDTAVYYCARESIGGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 273 - DNA, 1344 bp, Homo sapiens
caggtgcagctgctggaatcaggcggcggactggtgcagcctggcggtagcctgagactgagctgcgctgctagtggcttcaccttccctactcacggcctgcactgggtcagacaggcccctggtaaaggcctggagtgggtcagcgctattagctacgagggtagcgagactaactacgccgatagcgtgaagggccggttcactatctctagggataactctaagaacaccctgtacctgcagatgaatagcctgagagccgaggacaccgccgtctactactgcgctagagagtctatcggcggctacttcgactactggggtcagggcaccctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 274 - protein, 11 aa, Homo sapiens
SGDALGKNTVS

SEQ ID NO: 275 - protein, 7 aa, Homo sapiens
DDTDRPS

SEQ ID NO: 276 - protein, 9 aa, Homo sapiens
SSTDLSTVV

SEQ ID NO: 277 - protein, 11 aa, Homo sapiens
SGDALGKNTVS

SEQ ID NO: 278 - protein, 7 aa, Homo sapiens
DDTDRPS

SEQ ID NO: 279 - protein, 9 aa, Homo sapiens
SSTDLSTVV

SEQ ID NO: 280 - protein, 7 aa, Homo sapiens
DALGKNT

SEQ ID NO: 281 - skipped

SEQ ID NO: 282 - protein, 6 aa, Homo sapiens
TDLSTV

SEQ ID NO: 283 - protein, 105 aa, Homo sapiens
SYELTQPLSVSVALGQTARITCSGDALGKNTVSWYQQKPGQAPVLVIYDDTDRPSGIPERFSGSNSGNTATLTISRAQAGDEADYYCSSTDLSTWFGGGTKLTVL

SEQ ID NO: 284 - DNA, 318 bp, Homo sapiens; identical to SEQ ID NO: 232

SEQ ID NO: 285 - protein, 212 aa, Homo sapiens; identical to SEQ ID NO: 233

SEQ ID NO: 286 - DNA, 636 bp, Homo sapiens; identical to SEQ ID NO: 234

SEQ ID NO: 287 - protein, 10 aa, Homo sapiens
GFTFPTHGLH

SEQ ID NO: 288 - protein, 17 aa, Homo sapiens
AISYEGSETNYADSVKG

SEQ ID NO: 289 - protein, 9 aa, Homo sapiens
ESIGGYFDY

SEQ ID NO: 290 - protein, 5 aa, Homo sapiens
THGLH

SEQ ID NO: 291 - protein, 17 aa, Homo sapiens
AISYEGSETNYADSVKG

SEQ ID NO: 292 - protein, 9 aa, Homo sapiens
ESIGGYFDY

SEQ ID NO: 293 - protein, 7 aa, Homo sapiens
GFTFPTH

SEQ ID NO: 294 - protein, 6 aa, Homo sapiens
SYEGSE

SEQ ID NO: 295 - protein, 9 aa, Homo sapiens
ESIGGYFDY

SEQ ID NO: 296 - protein, 118 aa, Homo sapiens; identical to SEQ ID NO: 270

SEQ ID NO: 297 - DNA, 354 bp, Homo sapiens
caggtgcaattgctggaaagcggcggtggcctggtgcagccgggtggcagcctgcgtctgagctgcgcggcgtccggattcacctttcctactcatggtctgcattgggtgcgccaggccccgggcaaaggtctcgagtgggtttccgctatctcttacgagggttctgaaaccaactatgcggatagcgtgaaaggccgctttaccatcagccgcgataattcgaaaaacaccctgtatctgcaaatgaacagcctgcgtgcggaagatacggccgtgtattattgcgcgcgtgaatctatcggtggttacttcgattactggggccaaggcaccctggtgactgttagctca

SEQ ID NO: 298 - protein, 448 aa, Homo sapiens
QVQLLESGGGLVQPGGSLRLSCAASGFTFPTHGLHVWRQAPGKGLEWVSAISYEGSETNYADSVKGRFTISRDNSKNTLYLQMNSLRAEDTAVYYCARESIGGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 299 - DNA, 1344 bp, Homo sapiens
caggtgcaattgctggaaagcggcggtggcctggtgcagccgggtggcagcctgcgtctgagctgcgcggcgtccggattcacctttcctactcatggtctgcattgggtgcgccaggccccgggcaaaggtctcgagtgggtttccgctatctcttacgagggttctgaaaccaactatgcggatagcgtgaaaggccgctttaccatcagccgcgataattcgaaaaacaccctgtatctgcaaatgaacagcctgcgtgcggaagatacggccgtgtattattgcgcgcgtgaatctatcggtggttacttcgattactggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 300 - protein, 11 aa, Homo sapiens
SGDALGKNTVS

SEQ ID NO: 301 - protein, 7 aa, Homo sapiens
DDTDRPS

SEQ ID NO: 302 - protein, 9 aa, Homo sapiens
SSTDLSTVV

SEQ ID NO: 303 - protein, 11 aa, Homo sapiens
SGDALGKNTVS

SEQ ID NO: 304 - protein, 7 aa, Homo sapiens
DDTDRPS

SEQ ID NO: 305 - protein, 9 aa, Homo sapiens
SSTDLSTVV

SEQ ID NO: 306 - protein, 7 aa, Homo sapiens
DALGKNT

SEQ ID NO: 307 - skipped

SEQ ID NO: 308 - protein, 6 aa, Homo sapiens
TDLSTV

SEQ ID NO: 309 - protein, 106 aa, Homo sapiens; identical to SEQ ID NO: 231

SEQ ID NO: 310 - DNA, 318 bp, Homo sapiens; identical to SEQ ID NO: 258

SEQ ID NO: 311 - protein, 212 aa, Homo sapiens; identical to SEQ ID NO: 233

SEQ ID NO: 312 - DNA, 636 bp, Homo sapiens; identical to SEQ ID NO: 260

SEQ ID NO: 313 - protein, 10 aa, Homo sapiens
GGTFRDYAIS

SEQ ID NO: 314 - protein, 17 aa, Homo sapiens
GIIPAFGTANYAQKFQG

SEQ ID NO: 315 - protein, 18 aa, Homo sapiens
EQDPEFGYGGYPYEAMDV

SEQ ID NO: 316 - protein, 5 aa, Homo sapiens
DYAIS

SEQ ID NO: 317 - protein, 17 aa, Homo sapiens
GIIPAFGTANYAQKFQG

SEQ ID NO: 318 - protein, 18 aa, Homo sapiens
EQDPEFGYGGYPYEAMDV

SEQ ID NO: 319 - protein, 7 aa, Homo sapiens
GGTFRDY

SEQ ID NO: 320 - protein, 6 aa, Homo sapiens
IPAFGT

SEQ ID NO: 321 - protein, 18 aa, Homo sapiens
EQDPEFGYGGYPYEAMDV

SEQ ID NO: 322 - protein, 127 aa, Homo sapiens
QVQLVQSGAEVKKPGSSVKVSCKASGGTFRDYAISVWRQAPGQGLEWMGGIIPAFGTANYAQKFQGRVTITADESTSTAYMELSSLRSEDTAVYYCAREQDPEFGYGGYPYEAMPVWGQGTLVTVSS

SEQ ID NO: 323 - DNA, 381 bp, Homo sapiens
caggtgcagctggtgcagtcaggcgccgaagtgaagaaacccggctctagcgtgaaagtcagctgtaaagctagtggcggcacctttagagactacgctattagctgggtcagacaggccccaggtcagggcctggagtggatgggcggaattatccccgccttcggcaccgctaactacgctcagaaatttcagggtagagtgactatcaccgccgacgagtctactagcaccgcctatatggaactgtctagcctgagatcagaggacaccgccgtctactactgcgctagagagcaggaccccgagttcggctacggcggctacccctacgaggctatggacgtgtggggtcagggcaccctggtcaccgtgtctagc

SEQ ID NO: 324 - protein, 457 aa, Homo sapiens
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPEFGYGGYPYEAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 325 - DNA, 1371 bp, Homo sapiens
caggtgcagctggtgcagtcaggcgccgaagtgaagaaacccggctctagcgtgaaagtcagctgtaaagctagtggcggcacctttagagactacgctattagctgggtcagacaggccccaggtcagggcctggagtggatgggcggaattatccccgccttcggcaccgctaactacgctcagaaatttcagggtagagtgactatcaccgccgacgagtctactagcaccgcctatatggaactgtctagcctgagatcagaggacaccgccgtctactactgcgctagagagcaggaccccgagttcggctacggcggctacccctacgaggctatggacgtgtggggtcagggcaccctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 326 - protein, 11 aa, Homo sapiens
SGDNIPQHSVH

SEQ ID NO: 327 - protein, 7 aa, Homo sapiens
DDTERPS

SEQ ID NO: 328 - protein, 11 aa, Homo sapiens
SSWDSSMDSVV

SEQ ID NO: 329 - protein, 11 aa, Homo sapiens
SGDNIPQHSVH

SEQ ID NO: 330 - protein, 7 aa, Homo sapiens
DDTERPS

SEQ ID NO: 331 - protein, 11 aa, Homo sapiens
SSWDSSMDSVV

SEQ ID NO: 332 - protein, 7 aa, Homo sapiens
DNIPQHS

SEQ ID NO: 333 - skipped

SEQ ID NO: 334 - protein, 8 aa, Homo sapiens
WDSSMDSV

SEQ ID NO: 335 - protein, 108 aa, Homo sapiens
SYELTQPLSVSVALGQTARITCSGDNIPQHSVHWVQQKPGQAPVLVIYDDTERPSGIPERFSGSNSGNTATLTISRAQAGDEADYYCSSWDSSMDSVVFGGGTKLTVL

SEQ ID NO: 336 - DNA, 324 bp, Homo sapiens; identical to SEQ ID NO: 180

SEQ ID NO: 337 - protein, 213 aa, Homo sapiens
SYELTQPLSVSVALGQTARITCSGDNIPQHSVHWVQQKPGQAPVLVIYDDTERPSGIPERFSGSNSGNTATLTISRAQAGDEADYYCSSWDSSMDSWFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 338 - DNA, 642 bp, Homo sapiens; identical to SEQ ID NO: 182

SEQ ID NO: 339 - protein, 10 aa, Homo sapiens
GGTFRDYAIS

SEQ ID NO: 340 - protein, 17 aa, Homo sapiens
GIIPAFGTANYAQKFQG

SEQ ID NO: 341 - protein, 18 aa, Homo sapiens
EQDPEFGYGGYPYEAMDV

SEQ ID NO: 342 - protein, 5 aa, Homo sapiens
DYAIS

SEQ ID NO: 343 - protein, 17 aa, Homo sapiens
GIIPAFGTANYAQKFQG

SEQ ID NO: 344 - protein, 18 aa, Homo sapiens
EQDPEFGYGGYPYEAMDV

SEQ ID NO: 345 - protein, 7 aa, Homo sapiens
GGTFRDY

SEQ ID NO: 346 - protein, 6 aa, Homo sapiens
IPAFGT

SEQ ID NO: 347 - protein, 18 aa, Homo sapiens
EQDPEFGYGGYPYEAMDV

SEQ ID NO: 348 - protein, 127 aa, Homo sapiens
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPEFGYGGYPYEAMDVWGQGTLVTVSS

SEQ ID NO: 349 - DNA, 381 bp, Homo sapiens
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacaggacccggaattcggttacggtggttacccgtatgaagctatggatgtttggggccaaggcaccctggtgactgttagctca

SEQ ID NO: 350 - protein, 457 aa, Homo sapiens
QVQLVQSGAEVKKPGSSVKVSCKASGGTFRDYAISVWRQAPGQGLEWMGGIIPAFGTANYAQKFQGRVTITADESTSTAYMELSSLRSEDTAVYYCAREQDPEFGYGGYPYEAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVQKSRWQQGNVFSCSVMHEALHNHYTQKSLSLSPGK

SEQ ID NO: 351 - DNA, 1371 bp, Homo sapiens
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacaggacccggaattcggttacggtggttacccgtatgaagctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 352 - protein, 11 aa, Homo sapiens
SGDNIPQHSVH

SEQ ID NO: 353 - protein, 7 aa, Homo sapiens
DDTERPS

SEQ ID NO: 354 - protein, 11 aa, Homo sapiens
SSWDSSMDSVV

SEQ ID NO: 355 - protein, 11 aa, Homo sapiens
SGDNIPQHSVH

SEQ ID NO: 356 - protein, 7 aa, Homo sapiens
DDTERPS

SEQ ID NO: 357 - protein, 11 aa, Homo sapiens
SSWDSSMDSVV

SEQ ID NO: 358 - protein, 7 aa, Homo sapiens
DNIPQHS

SEQ ID NO: 359 - skipped

SEQ ID NO: 360 - protein, 8 aa, Homo sapiens
WDSSMDSV

SEQ ID NO: 361 - protein, 108 aa, Homo sapiens
SYELTQPLSVSVALGQTARITCSGDNIPQHSVHWYQQKPGQAPVLVIYDDTERPSGIPERFSGSNSGNTATLTISRAQAGDEADYYCSSWDSSMDSVVFGGGTKLTVL

SEQ ID NO: 362 - DNA, 324 bp, Homo sapiens; identical to SEQ ID NO: 206

SEQ ID NO: 363 - protein, 214 aa, Homo sapiens; identical to SEQ ID NO: 181

SEQ ID NO: 364 - DNA, 642 bp, Homo sapiens; identical to SEQ ID NO: 208

SEQ ID NO: 365 - protein, 10 aa, Homo sapiens
GGTFRDYAIS

SEQ ID NO: 366 - protein, 17 aa, Homo sapiens
GIIPAFGTANYAQKFQG

SEQ ID NO: 367 - protein, 18 aa, Homo sapiens
EQDPEAGYGGYPYEAMDV

SEQ ID NO: 368 - protein, 5 aa, Homo sapiens
DYAIS

SEQ ID NO: 369 - protein, 17 aa, Homo sapiens
GIIPAFGTANYAQKFQG

SEQ ID NO: 370 - protein, 18 aa, Homo sapiens
EQDPEAGYGGYPYEAMDV

SEQ ID NO: 371 - protein, 7 aa, Homo sapiens
GGTFRDY

SEQ ID NO: 372 - protein, 6 aa, Homo sapiens
IPAFGT

SEQ ID NO: 373 - protein, 18 aa, Homo sapiens
EQDPEAGYGGYPYEAMDV

SEQ ID NO: 374 - protein, 127 aa, Homo sapiens
QVQLVQSGAEVKKPGSSVKVSCKASGGTFRDYAISVWRQAPGQGLEWMGGIIPAFGTANYAQKFQGRVTITADESTSTAYMELSSLRSEDTAVYYCAREQDPEAGYGGYPYEAMDVWGQGTLVTVSS

SEQ ID NO: 375 - DNA, 381 bp, Homo sapiens
caggtgcagctggtgcagtcaggcgccgaagtgaagaaacccggctctagcgtgaaagtcagctgtaaagctagtggcggcacctttagagactacgctattagctgggtcagacaggccccaggtcagggcctggagtggatgggcggaattatccccgccttcggcaccgctaactacgctcagaaatttcagggtagagtgactatcaccgccgacgagtctactagcaccgcctatatggaactgtctagcctgagatcagaggacaccgccgtctactactgcgctagagagcaggaccccgaggccggctacggcggctacccctacgaggctatggacgtgtggggtcagggcaccctggtcaccgtgtctagc

SEQ ID NO: 376 - protein, 457 aa, Homo sapiens
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPEAGYGGYPYEAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 377 - DNA, 1371 bp, Homo sapiens
caggtgcagctggtgcagtcaggcgccgaagtgaagaaacccggctctagcgtgaaagtcagctgtaaagctagtggcggcacctttagagactacgctattagctgggtcagacaggccccaggtcagggcctggagtggatgggcggaattatccccgccttcggcaccgctaactacgctcagaaatttcagggtagagtgactatcaccgccgacgagtctactagcaccgcctatatggaactgtctagcctgagatcagaggacaccgccgtctactactgcgctagagagcaggaccccgaggccggctacggcggctacccctacgaggctatggacgtgtggggtcagggcaccctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 378 - protein, 11 aa, Homo sapiens
SGDNIPQHSVH

SEQ ID NO: 379 - protein, 7 aa, Homo sapiens
DDTERPS

SEQ ID NO: 380 - protein, 11 aa, Homo sapiens
SSWDSSMDSVV

SEQ ID NO: 381 - protein, 11 aa, Homo sapiens
SGDNIPQHSVH

SEQ ID NO: 382 - protein, 7 aa, Homo sapiens
DDTERPS

SEQ ID NO: 383 - protein, 11 aa, Homo sapiens
SSWDSSMDSVV

SEQ ID NO: 384 - protein, 7 aa, Homo sapiens
DNIPQHS

SEQ ID NO: 385 - skipped

SEQ ID NO: 386 - protein, 8 aa, Homo sapiens
WDSSMDSV

SEQ ID NO: 387 - protein, 108 aa, Homo sapiens; identical to SEQ ID NO: 335

SEQ ID NO: 388 - DNA, 324 bp, Homo sapiens; identical to SEQ ID NO: 180

SEQ ID NO: 389 - protein, 213 aa, Homo sapiens; identical to SEQ ID NO: 337

SEQ ID NO: 390 - DNA, 642 bp, Homo sapiens; identical to SEQ ID NO: 182

SEQ ID NO: 391 - protein, 10 aa, Homo sapiens
GGTFRDYAIS

SEQ ID NO: 392 - protein, 17 aa, Homo sapiens
GIIPAFGTANYAQKFQG

SEQ ID NO: 393 - protein, 18 aa, Homo sapiens
EQDPEAGYGGYPYEAMDV

SEQ ID NO: 394 - protein, 5 aa, Homo sapiens
DYAIS

SEQ ID NO: 395 - protein, 17 aa, Homo sapiens
GIIPAFGTANYAQKFQG

SEQ ID NO: 396 - protein, 18 aa, Homo sapiens
EQDPEAGYGGYPYEAMDV

SEQ ID NO: 397 - protein, 7 aa, Homo sapiens
GGTFRDY

SEQ ID NO: 398 - protein, 6 aa, Homo sapiens
IPAFGT

SEQ ID NO: 399 - protein, 18 aa, Homo sapiens
EQDPEAGYGGYPYEAMDV

SEQ ID NO: 400 - protein, 127 aa, Homo sapiens
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPEAGYGGYPYEAMDVWGQGTLVTVSS

SEQ ID NO: 401 - DNA, 381 bp, Homo sapiens
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacaggacccggaagccggttacggtggttacccgtatgaagctatggatgtttggggccaaggcaccctggtgactgttagctca

SEQ ID NO: 402 - protein, 457 aa, Homo sapiens
QVQLVQSGAEVKKPGSSVKVSCKASGGTFRDYAISVWRQAPGQGLEWMGGIIPAFGTANYAQKFQGRVTITADESTSTAYMELSSLRSEDTAVYYCAREQDPEAGYGGYPYEAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 403 - DNA, 1371 bp, Homo sapiens
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacaggacccggaagccggttacggtggttacccgtatgaagctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 404 - protein, 11 aa, Homo sapiens
SGDNIPQHSVH

SEQ ID NO: 405 - protein, 7 aa, Homo sapiens
DDTERPS

SEQ ID NO: 406 - protein, 11 aa, Homo sapiens
SSWDSSMDSVV

SEQ ID NO: 407 - protein, 11 aa, Homo sapiens
SGDNIPQHSVH

SEQ ID NO: 408 - protein, 7 aa, Homo sapiens
DDTERPS

SEQ ID NO: 409 - protein, 11 aa, Homo sapiens
SSWDSSMDSVV

SEQ ID NO: 410 - protein, 7 aa, Homo sapiens
DNIPQHS

SEQ ID NO: 411 - skipped

SEQ ID NO: 412 - protein, 8 aa, Homo sapiens
WDSSMDSV

SEQ ID NO: 413 - protein, 107 aa, Homo sapiens
SYELTQPLSVSVALGQTARITCSGDNIPQHSVHVWQQKPGQAPVLVIYDDTERPSGIPERFSGSNSGNTATLTISRAQAGDEADYYCSSWDSSMDSWFGGGTKLTVL

SEQ ID NO: 414 - DNA, 324 bp, Homo sapiens; identical to SEQ ID NO: 206

SEQ ID NO: 415 - protein, 213 aa, Homo sapiens
SYELTQPLSVSVALGQTARITCSGDNIPQHSVHWYQQKPGQAPVLVIYDDTERPSGIPERFSGSNSGNTATLTISRAQAGDEADYYCSSWDSSMDSWFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 416 - DNA, 642 bp, Homo sapiens; identical to SEQ ID NO: 208

SEQ ID NO: 417 - protein, 10 aa, Homo sapiens
GGTFRDYAIS

SEQ ID NO: 418 - protein, 17 aa, Homo sapiens
GIIPAFGTANYAQKFQG

SEQ ID NO: 419 - protein, 18 aa, Homo sapiens
EQDPESGYGGYPYEAMDV

SEQ ID NO: 420 - protein, 5 aa, Homo sapiens
DYAIS

SEQ ID NO: 421 - protein, 17 aa, Homo sapiens
GIIPAFGTANYAQKFQG

SEQ ID NO: 422 - protein, 18 aa, Homo sapiens
EQDPESGYGGYPYEAMDV

SEQ ID NO: 423 - protein, 7 aa, Homo sapiens
GGTFRDY

SEQ ID NO: 424 - protein, 6 aa, Homo sapiens
IPAFGT

SEQ ID NO: 425 - protein, 18 aa, Homo sapiens
EQDPESGYGGYPYEAMDV

SEQ ID NO: 426 - protein, 127 aa, Homo sapiens
QVQLVQSGAEVKKPGSSVKVSCKASGGTFRDYAISVWRQAPGQGLEWMGGIIPAFGTANYAQKFQGRVTITADESTSTAYMELSSLRSEDTAVYYCAREQDPESGYGGYPYEAMDVWGQGTLVTVSS

SEQ ID NO: 427 - DNA, 381 bp, Homo sapiens
caggtgcagctggtgcagtcaggcgccgaagtgaagaaacccggctctagcgtgaaagtcagctgtaaagctagtggcggcacctttagagactacgctattagctgggtcagacaggccccaggtcagggcctggagtggatgggcggaattatccccgccttcggcaccgctaactacgctcagaaatttcagggtagagtgactatcaccgccgacgagtctactagcaccgcctatatggaactgtctagcctgagatcagaggacaccgccgtctactactgcgctagagagcaggaccccgagtccggctacggcggctacccctacgaggctatggacgtgtggggtcagggcaccctggtcaccgtgtctagc

SEQ ID NO: 428 - protein, 457 aa, Homo sapiens
QVQLVQSGAEVKKPGSSVKVSCKASGGTFRDYAISVWRQAPGQGLEWMGGIIPAFGTANYAQKFQGRVTITADESTSTAYMELSSLRSEDTAVYYCAREQDPESGYGGYPYEAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 429 - DNA, 1371 bp, Homo sapiens
caggtgcagctggtgcagtcaggcgccgaagtgaagaaacccggctctagcgtgaaagtcagctgtaaagctagtggcggcacctttagagactacgctattagctgggtcagacaggccccaggtcagggcctggagtggatgggcggaattatccccgccttcggcaccgctaactacgctcagaaatttcagggtagagtgactatcaccgccgacgagtctactagcaccgcctatatggaactgtctagcctgagatcagaggacaccgccgtctactactgcgctagagagcaggaccccgagtccggctacggcggctacccctacgaggctatggacgtgtggggtcagggcaccctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 430 - protein, 11 aa, Homo sapiens
SGDNIPQHSVH

SEQ ID NO: 431 - protein, 7 aa, Homo sapiens
DDTERPS

SEQ ID NO: 432 - protein, 11 aa, Homo sapiens
SSWDSSMDSVV

SEQ ID NO: 433 - protein, 11 aa, Homo sapiens
SGDNIPQHSVH

SEQ ID NO: 434 - protein, 7 aa, Homo sapiens
DDTERPS

SEQ ID NO: 435 - protein, 11 aa, Homo sapiens
SSWDSSMDSVV

SEQ ID NO: 436 - protein, 7 aa, Homo sapiens
DNIPQHS

SEQ ID NO: 437 - skipped

SEQ ID NO: 438 - protein, 8 aa, Homo sapiens
WDSSMDSV

SEQ ID NO: 439 - protein, 108 aa, Homo sapiens; identical to SEQ ID NO: 361

SEQ ID NO: 440 - DNA, 324 bp, Homo sapiens; identical to SEQ ID NO: 180

SEQ ID NO: 441 - protein, 214 aa, Homo sapiens; identical to SEQ ID NO: 181

SEQ ID NO: 442 - DNA, 642 bp, Homo sapiens; identical to SEQ ID NO: 182

SEQ ID NO: 443 - protein, 10 aa, Homo sapiens
GGTFRDYAIS

SEQ ID NO: 444 - protein, 17 aa, Homo sapiens
GIIPAFGTANYAQKFQG

SEQ ID NO: 445 - protein, 18 aa, Homo sapiens
EQDPESGYGGYPYEAMDV

SEQ ID NO: 446 - protein, 5 aa, Homo sapiens
DYAIS

SEQ ID NO: 447 - protein, 17 aa, Homo sapiens
GIIPAFGTANYAQKFQG

SEQ ID NO: 448 - protein, 18 aa, Homo sapiens
EQDPESGYGGYPYEAMDV

SEQ ID NO: 449 - protein, 7 aa, Homo sapiens
GGTFRDY

SEQ ID NO: 450 - protein, 6 aa, Homo sapiens
IPAFGT

SEQ ID NO: 451 - protein, 18 aa, Homo sapiens
EQDPESGYGGYPYEAMDV

SEQ ID NO: 452 - protein, 127 aa, Homo sapiens
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPESGYGGYPYEAMDVWGQGTLVTVSS

SEQ ID NO: 453 - DNA, 381 bp, Homo sapiens
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacaggacccggaaagcggttacggtggttacccgtatgaagctatggatgtttggggccaaggcaccctggtgactgttagctca

SEQ ID NO: 454 - protein, 457 aa, Homo sapiens
QVQLVQSGAEVKKPGSSVKVSCKASGGTFRDYAISVWRQAPGQGLEWMGGIIPAFGTANYAQKFQGRVTITADESTSTAYMELSSLRSEDTAVYYCAREQDPESGYGGYPYEAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVQKSRWQQGNVFSCSVMHEALHNHYTQKSLSLSPGK

SEQ ID NO: 455 - DNA, 1371 bp, Homo sapiens
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacaggacccggaaagcggttacggtggttacccgtatgaagctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 456 - protein, 11 aa, Homo sapiens
SGDNIPQHSVH

SEQ ID NO: 457 - protein, 7 aa, Homo sapiens
DDTERPS

SEQ ID NO: 458 - protein, 11 aa, Homo sapiens
SSWDSSMDSVV

SEQ ID NO: 459 - protein, 11 aa, Homo sapiens
SGDNIPQHSVH

SEQ ID NO: 460 - protein, 7 aa, Homo sapiens
DDTERPS

SEQ ID NO: 461 - protein, 11 aa, Homo sapiens
SSWDSSMDSVV

SEQ ID NO: 462 - protein, 7 aa, Homo sapiens
DNIPQHS

SEQ ID NO: 463 - skipped

SEQ ID NO: 464 - protein, 8 aa, Homo sapiens
WDSSMDSV

SEQ ID NO: 465 - protein, 108 aa, Homo sapiens
SYELTQPLSVSVALGQTARITCSGDNIPQHSVHVWQQKPGQAPVLVIYDDTERPSGIPERFSGSNSGNTATLTISRAQAGDEADYYCSSWDSSMDSVVFGGGTKLTVL

SEQ ID NO: 466 - DNA, 324 bp, Homo sapiens; identical to SEQ ID NO: 206

SEQ ID NO: 467 - protein, 213 aa, Homo sapiens; identical to SEQ ID NO: 415

SEQ ID NO: 468 - DNA, 642 bp, Homo sapiens; identical to SEQ ID NO: 208

SEQ ID NO: 469 - protein, 10 aa, Homo sapiens
GGTFRDYAIS

SEQ ID NO: 470 - protein, 17 aa, Homo sapiens
GIIPAFGTANYAQKFQG

SEQ ID NO: 471 - protein, 18 aa, Homo sapiens
EQDPEYGFGGYPYEAMDV

SEQ ID NO: 472 - protein, 5 aa, Homo sapiens
DYAIS

SEQ ID NO: 473 - protein, 17 aa, Homo sapiens
GIIPAFGTANYAQKFQG

SEQ ID NO: 474 - protein, 18 aa, Homo sapiens
EQDPEYGFGGYPYEAMDV

SEQ ID NO: 475 - protein, 7 aa, Homo sapiens
GGTFRDY

SEQ ID NO: 476 - protein, 6 aa, Homo sapiens
IPAFGT

SEQ ID NO: 477 - protein, 18 aa, Homo sapiens
EQDPEYGFGGYPYEAMDV

SEQ ID NO: 478 - protein, 127 aa, Homo sapiens
QVQLVQSGAEVKKPGSSVKVSCKASGGTFRDYAISVWRQAPGQGLEWMGGIIPAFGTANYAQKFQGRVTITADESTSTAYMELSSLRSEDTAVYYCAREQDPEYGFGGYPYEAMDVWGQGTLVTVSS

SEQ ID NO: 479 - DNA, 381 bp, Homo sapiens
caggtgcagctggtgcagtcaggcgccgaagtgaagaaacccggctctagcgtgaaagtcagctgtaaagctagtggcggcacctttagagactacgctattagctgggtcagacaggccccaggtcagggcctggagtggatgggcggaattatccccgccttcggcaccgctaactacgctcagaaatttcagggtagagtgactatcaccgccgacgagtctactagcaccgcctatatggaactgtctagcctgagatcagaggacaccgccgtctactactgcgctagagagcaggaccccgagtacggcttcggcggctacccctacgaggctatggacgtgtggggtcagggcaccctggtcaccgtgtctagc

SEQ ID NO: 480 - protein, 457 aa, Homo sapiens
QVQLVQSGAEVKKPGSSVKVSCKASGGTFRDYAISVWRQAPGQGLEWMGGIIPAFGTANYAQKFQGRVTITADESTSTAYMELSSLRSEDTAVYYCAREQDPEYGFGGYPYEAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 481 - DNA, 1371 bp, Homo sapiens
caggtgcagctggtgcagtcaggcgccgaagtgaagaaacccggctctagcgtgaaagtcagctgtaaagctagtggcggcacctttagagactacgctattagctgggtcagacaggccccaggtcagggcctggagtggatgggcggaattatccccgccttcggcaccgctaactacgctcagaaatttcagggtagagtgactatcaccgccgacgagtctactagcaccgcctatatggaactgtctagcctgagatcagaggacaccgccgtctactactgcgctagagagcaggaccccgagtacggcttcggcggctacccctacgaggctatggacgtgtggggtcagggcaccctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 482 - protein, 11 aa, Homo sapiens
SGDNIPQHSVH

SEQ ID NO: 483 - protein, 7 aa, Homo sapiens
DDTERPS

SEQ ID NO: 484 - protein, 11 aa, Homo sapiens
SSWDSSMDSVV

SEQ ID NO: 485 - protein, 11 aa, Homo sapiens
SGDNIPQHSVH

SEQ ID NO: 486 - protein, 7 aa, Homo sapiens
DDTERPS

SEQ ID NO: 487 - protein, 11 aa, Homo sapiens
SSWDSSMDSVV

SEQ ID NO: 488 - protein, 7 aa, Homo sapiens
DNIPQHS

SEQ ID NO: 489 - skipped

SEQ ID NO: 490 - protein, 8 aa, Homo sapiens
WDSSMDSV

SEQ ID NO: 491 - protein, 108 aa, Homo sapiens; identical to SEQ ID NO: 361

SEQ ID NO: 492 - DNA, 324 bp, Homo sapiens; identical to SEQ ID NO: 180

SEQ ID NO: 493 - protein, 214 aa, Homo sapiens; identical to SEQ ID NO: 181

SEQ ID NO: 494 - DNA, 642 bp, Homo sapiens; identical to SEQ ID NO: 182

SEQ ID NO: 495 - protein, 10 aa, Homo sapiens
GGTFRDYAIS

SEQ ID NO: 496 - protein, 17 aa, Homo sapiens
GIIPAFGTANYAQKFQG

SEQ ID NO: 497 - protein, 18 aa, Homo sapiens
EQDPEYGFGGYPYEAMDV

SEQ ID NO: 498 - protein, 5 aa, Homo sapiens
DYAIS

SEQ ID NO: 499 - protein, 17 aa, Homo sapiens
GIIPAFGTANYAQKFQG

SEQ ID NO: 500 - protein, 18 aa, Homo sapiens
EQDPEYGFGGYPYEAMDV

SEQ ID NO: 501 - protein, 7 aa, Homo sapiens
GGTFRDY

SEQ ID NO: 502 - protein, 6 aa, Homo sapiens
IPAFGT

SEQ ID NO: 503 - protein, 18 aa, Homo sapiens
EQDPEYGFGGYPYEAMDV

SEQ ID NO: 504 - protein, 127 aa, Homo sapiens
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPEYGFGGYPYEAMDVWGQGTLVTVSS

SEQ ID NO: 505 - DNA, 381 bp, Homo sapiens
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacaggacccggaatacggtttcggtggttacccgtatgaagctatggatgtttggggccaaggcaccctggtgactgttagctca

SEQ ID NO: 506 - protein, 457 aa, Homo sapiens
QVQLVQSGAEVKKPGSSVKVSCKASGGTFRDYAISVWRQAPGQGLEWMGGIIPAFGTANYAQKFQGRVTITADESTSTAYMELSSLRSEDTAVYYCAREQDPEYGFGGYPYEAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVQKSRWQQGNVFSCSVMHEALHNHYTQKSLSLSPGK

SEQ ID NO: 507 - DNA, 1371 bp, Homo sapiens
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacaggacccggaatacggtttcggtggttacccgtatgaagctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 508 - protein, 11 aa, Homo sapiens
SGDNIPQHSVH

SEQ ID NO: 509 - protein, 7 aa, Homo sapiens
DDTERPS

SEQ ID NO: 510 - protein, 11 aa, Homo sapiens
SSWDSSMDSVV

SEQ ID NO: 511 - protein, 11 aa, Homo sapiens
SGDNIPQHSVH

SEQ ID NO: 512 - protein, 7 aa, Homo sapiens
DDTERPS

SEQ ID NO: 513 - protein, 11 aa, Homo sapiens
SSWDSSMDSVV

SEQ ID NO: 514 - protein, 7 aa, Homo sapiens
DNIPQHS

SEQ ID NO: 515 - skipped

SEQ ID NO: 516 - protein, 8 aa, Homo sapiens
WDSSMDSV

SEQ ID NO: 517 - protein, 108 aa, Homo sapiens
SYELTQPLSVSVALGQTARITCSGDNIPQHSVHWYQQKPGQAPVLVIYQDTERPSGIPERFSGSNSGNTATLTISRAQAGDEADYYCSSWDSSMDSVVFGGGTKLTVL

SEQ ID NO: 518 - DNA, 324 bp, Homo sapiens; identical to SEQ ID NO: 206

SEQ ID NO: 519 - protein, 213 aa, Homo sapiens
SYELTQPLSVSVALGQTARITCSGDNIPQHSVHWYQQKPGQAPVLVIYDDTERPSGIPERFSGSNSGNTATLTISRAQAGDEADYYCSSWDSSMDSWFGGGTKLTVLGQPKAAPSVTLFPPSSEELQANKATLVCLISDFYPGAVTVAWKADSSPVKAGVETTTPSKQSNNKYAASSYLSLTPEGWKSHRSYSCQVTHEGSTVEKTVAPTECS

SEQ ID NO: 520 - DNA, 642 bp, Homo sapiens; identical to SEQ ID NO: 208

SEQ ID NO: 521 - protein, 10 aa, Homo sapiens
GGTFRDYAIS

SEQ ID NO: 522 - protein, 17 aa, Homo sapiens
GIIPAFGTANYAQKFQG

SEQ ID NO: 523 - protein, 18 aa, Homo sapiens
EQDPEYGYGGFPVEAMDV

SEQ ID NO: 524 - protein, 5 aa, Homo sapiens
DYAIS

SEQ ID NO: 525 - protein, 17 aa, Homo sapiens
GIIPAFGTANYAQKFQG

SEQ ID NO: 526 - protein, 18 aa, Homo sapiens
EQDPEYGYGGFPYEAMDV

SEQ ID NO: 527 - protein, 7 aa, Homo sapiens
GGTFRDY

SEQ ID NO: 528 - protein, 6 aa, Homo sapiens
IPAFGT

SEQ ID NO: 529 - protein, 18 aa, Homo sapiens
EQDPEYGYGGFPYEAMDV

SEQ ID NO: 530 - protein, 127 aa, Homo sapiens
QVQLVQSGAEVKKPGSSVKVSCKASGGTFRDYAISVWRQAPGQGLEWMGGIIPAFGTANYAQKFQGRVTITADESTSTAYMELSSLRSEDTAVYYCAREQDPEYGYGGFPYEAMDVWGQGTLVTVSS

SEQ ID NO: 531 - DNA, 381 bp, Homo sapiens
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacaggacccggaatacggttacggtggtttcccgtatgaagctatggatgtttggggccaaggcaccctggtgactgttagctca

SEQ ID NO: 532 - protein, 457 aa, Homo sapiens
QVQLVQSGAEVKKPGSSVKVSCKASGGTFRDYAISVWRQAPGQGLEWMGGIIPAFGTANYAQKFQGRVTITADESTSTAYMELSSLRSEDTAVYYCAREQDPEYGYGGFPYEAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 533 - DNA, 1371 bp, Homo sapiens
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacaggacccggaatacggttacggtggtttcccgtatgaagctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 534 - protein, 11 aa, Homo sapiens
SGDNIPQHSVH

SEQ ID NO: 535 - protein, 7 aa, Homo sapiens
DDTERPS

SEQ ID NO: 536 - protein, 11 aa, Homo sapiens
SSWDSSMDSVV

SEQ ID NO: 537 - protein, 11 aa, Homo sapiens
SGDNIPQHSVH

SEQ ID NO: 538 - protein, 7 aa, Homo sapiens
DDTERPS

SEQ ID NO: 539 - protein, 11 aa, Homo sapiens
SSWDSSMDSVV

SEQ ID NO: 540 - protein, 7 aa, Homo sapiens
DNIPQHS

SEQ ID NO: 541 - skipped

SEQ ID NO: 542 - protein, 8 aa, Homo sapiens
WDSSMDSV

SEQ ID NO: 543 - protein, 108 aa, Homo sapiens; identical to SEQ ID NO: 361

SEQ ID NO: 544 - DNA, 324 bp, Homo sapiens; identical to SEQ ID NO: 206

SEQ ID NO: 545 - protein, 214 aa, Homo sapiens; identical to SEQ ID NO: 181

SEQ ID NO: 546 - DNA, 642 bp, Homo sapiens; identical to SEQ ID NO: 208

SEQ ID NO: 547 - protein, 10 aa, Homo sapiens
GGTFRDYAIS

SEQ ID NO: 548 - protein, 17 aa, Homo sapiens
GIIPAFGTANYAQKFQG

SEQ ID NO: 549 - protein, 18 aa, Homo sapiens
EQDPEYGYGGYPFEAMDV

SEQ ID NO: 550 - protein, 5 aa, Homo sapiens
DYAIS

SEQ ID NO: 551 - protein, 17 aa, Homo sapiens
GIIPAFGTANYAQKFQG

SEQ ID NO: 552 - protein, 18 aa, Homo sapiens
EQDPEYGYGGYPFEAMDV

SEQ ID NO: 553 - protein, 7 aa, Homo sapiens
GGTFRDY

SEQ ID NO: 554 - protein, 6 aa, Homo sapiens
IPAFGT

SEQ ID NO: 555 - protein, 18 aa, Homo sapiens
EQDPEYGYGGYPFEAMDV

SEQ ID NO: 556 - protein, 127 aa, Homo sapiens
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPEYGYGGYPFEAMDVWGQGTLVTVSS

SEQ ID NO: 557 - DNA, 381 bp, Homo sapiens
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacaggacccggaatacggttacggtggttacccgttcgaagctatggatgtttggggccaaggcaccctggtgactgttagctca

SEQ ID NO: 558 - protein, 457 aa, Homo sapiens
QVQLVQSGAEVKKPGSSVKVSCKASGGTFRDYAISVWRQAPGQGLEWMGGIIPAFGTANYAQKFQGRVTITADESTSTAYMELSSLRSEDTAVYYCAREQDPEYGYGGYPFEAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVQKSRWQQGNVFSCSVMHEALHNHYTQKSLSLSPGK

SEQ ID NO: 559 - DNA, 1371 bp, Homo sapiens
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacaggacccggaatacggttacggtggttacccgttcgaagctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 560 - protein, 11 aa, Homo sapiens
SGDNIPQHSVH

SEQ ID NO: 561 - protein, 7 aa, Homo sapiens
DDTERPS

SEQ ID NO: 562 - protein, 11 aa, Homo sapiens
SSWDSSMDSVV

SEQ ID NO: 563 - protein, 11 aa, Homo sapiens
SGDNIPQHSVH

SEQ ID NO: 564 - protein, 7 aa, Homo sapiens
DDTERPS

SEQ ID NO: 565 - protein, 11 aa, Homo sapiens
SSWDSSMDSVV

SEQ ID NO: 566 - protein, 7 aa, Homo sapiens
DNIPQHS

SEQ ID NO: 567 - skipped

SEQ ID NO: 568 - protein, 8 aa, Homo sapiens
WDSSMDSV

SEQ ID NO: 569 - protein, 108 aa, Homo sapiens; identical to SEQ ID NO: 361

SEQ ID NO: 570 - DNA, 324 bp, Homo sapiens; identical to SEQ ID NO: 206

SEQ ID NO: 571 - protein, 214 aa, Homo sapiens; identical to SEQ ID NO: 181

SEQ ID NO: 572 - DNA, 642 bp, Homo sapiens; identical to SEQ ID NO: 208

SEQ ID NO: 573 - protein, 10 aa, Homo sapiens
GGTFRDYAIS

SEQ ID NO: 574 - protein, 17 aa, Homo sapiens
GIIPAFGTANYAQKFQG

SEQ ID NO: 575 - protein, 18 aa, Homo sapiens
EQDPSYGYGGYPYEAMDV

SEQ ID NO: 576 - protein, 5 aa, Homo sapiens
DYAIS

SEQ ID NO: 577 - protein, 17 aa, Homo sapiens
GIIPAFGTANYAQKFQG

SEQ ID NO: 578 - protein, 18 aa, Homo sapiens
EQDPSYGYGGYPYEAMDV

SEQ ID NO: 579 - protein, 7 aa, Homo sapiens
GGTFRDY

SEQ ID NO: 580 - protein, 6 aa, Homo sapiens
IPAFGT

SEQ ID NO: 581 - protein, 18 aa, Homo sapiens
EQDPSYGYGGYPYEAMDV

SEQ ID NO: 582 - protein, 127 aa, Homo sapiens
QVQLVQSGAEVKKPGSSVKVSCKASGGTFRDYAISVWRQAPGQGLEWMGGIIPAFGTANYAQKFQGRVTITADESTSTAYMELSSLRSEDTAVYYCAREQDPSYGYGGYPYEAMDVWGQGTLVTVSS

SEQ ID NO: 583 - DNA, 381 bp, Homo sapiens
caggtgcagctggtgcagtcaggcgccgaagtgaagaaacccggctctagcgtgaaagtcagctgtaaagctagtggcggcacctttagagactacgctattagctgggtcagacaggccccaggtcagggcctggagtggatgggcggaattatccccgccttcggcaccgctaactacgctcagaaatttcagggtagagtgactatcaccgccgacgagtctactagcaccgcctatatggaactgtctagcctgagatcagaggacaccgccgtctactactgcgctagagagcaggacccctcctacggctacggcggctacccctacgaggctatggacgtgtggggtcagggcaccctggtcaccgtgtctagc

SEQ ID NO: 584 - protein, 457 aa, Homo sapiens
QVQLVQSGAEVKKPGSSVKVSCKASGGTFRDYAISVWRQAPGQGLEWMGGIIPAFGTANYAQKFQGRVTITADESTSTAYMELSSLRSEDTAVYYCAREQDPSYGYGGYPYEAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 585 - DNA, 1371 bp, Homo sapiens
caggtgcagctggtgcagtcaggcgccgaagtgaagaaacccggctctagcgtgaaagtcagctgtaaagctagtggcggcacctttagagactacgctattagctgggtcagacaggccccaggtcagggcctggagtggatgggcggaattatccccgccttcggcaccgctaactacgctcagaaatttcagggtagagtgactatcaccgccgacgagtctactagcaccgcctatatggaactgtctagcctgagatcagaggacaccgccgtctactactgcgctagagagcaggacccctcctacggctacggcggctacccctacgaggctatggacgtgtggggtcagggcaccctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 586 - protein, 11 aa, Homo sapiens
SGDNIPQHSVH

SEQ ID NO: 587 - protein, 7 aa, Homo sapiens
DDTERPS

SEQ ID NO: 588 - protein, 11 aa, Homo sapiens
SSWDSSMDSVV

SEQ ID NO: 589 - protein, 11 aa, Homo sapiens
SGDNIPQHSVH

SEQ ID NO: 590 - protein, 7 aa, Homo sapiens
DDTERPS

SEQ ID NO: 591 - protein, 11 aa, Homo sapiens
SSWDSSMDSVV

SEQ ID NO: 592 - protein, 7 aa, Homo sapiens
DNIPQHS

SEQ ID NO: 593 - skipped

SEQ ID NO: 594 - protein, 8 aa, Homo sapiens
WDSSMDSV

SEQ ID NO: 595 - protein, 107 aa, Homo sapiens; identical to SEQ ID NO: 179

SEQ ID NO: 596 - DNA, 324 bp, Homo sapiens; identical to SEQ ID NO: 180

SEQ ID NO: 597 - protein, 214 aa, Homo sapiens; identical to SEQ ID NO: 181

SEQ ID NO: 598 - DNA, 642 bp, Homo sapiens; identical to SEQ ID NO: 182

SEQ ID NO: 599 - protein, 10 aa, Homo sapiens
GGTFRDYAIS

SEQ ID NO: 600 - protein, 17 aa, Homo sapiens
GIIPAFGTANYAQKFQG

SEQ ID NO: 601 - protein, 18 aa, Homo sapiens
EQDPSYGYGGYPYEAMDV

SEQ ID NO: 602 - protein, 5 aa, Homo sapiens
DYAIS

SEQ ID NO: 603 - protein, 17 aa, Homo sapiens
GIIPAFGTANYAQKFQG

SEQ ID NO: 604 - protein, 18 aa, Homo sapiens
EQDPSYGYGGYPYEAMDV

SEQ ID NO: 605 - protein, 7 aa, Homo sapiens
GGTFRDY

SEQ ID NO: 606 - protein, 6 aa, Homo sapiens
IPAFGT

SEQ ID NO: 607 - protein, 18 aa, Homo sapiens
EQDPSYGYGGYPYEAMDV

SEQ ID NO: 608 - protein, 127 aa, Homo sapiens
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQDPSYGYGGYPYEAMDVWGQGTLVTVSS

SEQ ID NO: 609 - DNA, 381 bp, Homo sapiens
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacaggacccgagctacggttacggtggttacccgtatgaagctatggatgtttggggccaaggcaccctggtgactgttagctca

SEQ ID NO: 610 - protein, 457 aa, Homo sapiens
QVQLVQSGAEVKKPGSSVKVSCKASGGTFRDYAISVWRQAPGQGLEWMGGIIPAFGTANYAQKFQGRVTITADESTSTAYMELSSLRSEDTAVYYCAREQDPSYGYGGYPYEAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVQKSRWQQGNVFSCSVMHEALHNHYTQKSLSLSPGK

SEQ ID NO: 611 - DNA, 1371 bp, Homo sapiens
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacaggacccgagctacggttacggtggttacccgtatgaagctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 612 - protein, 11 aa, Homo sapiens
SGDNIPGHGVH

SEQ ID NO: 613 - protein, 7 aa, Homo sapiens
DDTERPS

SEQ ID NO: 614 - protein, 11 aa, Homo sapiens
SSWDSSMDSVV

SEQ ID NO: 615 - protein, 11 aa, Homo sapiens
SGDNIPQHSVH

SEQ ID NO: 616 - protein, 7 aa, Homo sapiens
DDTERPS

SEQ ID NO: 617 - protein, 11 aa, Homo sapiens
SSWDSSMDSVV

SEQ ID NO: 618 - protein, 7 aa, Homo sapiens
DNIPQHS

SEQ ID NO: 619 - skipped

SEQ ID NO: 620 - protein, 8 aa, Homo sapiens
WDSSMDSV

SEQ ID NO: 621 - protein, 107 aa, Homo sapiens; identical to SEQ ID NO: 413

SEQ ID NO: 622 - DNA, 324 bp, Homo sapiens; identical to SEQ ID NO: 206

SEQ ID NO: 623 - protein, 214 aa, Homo sapiens
SYELTQPLSVSVALGQTARITCSGDNIPQHSVHWYQQKPGQAPVLVIYDDTERPSGIPERFSGSNSGNTATLTISRAQAGDEADYYCSSWDSSMDSVVFGGGTKLTVLGQPKAAPSVTLFPPSSEELQANKATLVCLISDFYPGAVTVAWKADSSPVKAGVETTTPSKQSNNKYAASSYLSLTPEGWKSHRSYSCQVTHEGSTVEKTVAPTECS

SEQ ID NO: 624 - DNA, 642 bp, Homo sapiens; identical to SEQ ID NO: 208

SEQ ID NO: 625 - protein, 10 aa, Homo sapiens
GGTFRDYAIS

SEQ ID NO: 626 - protein, 17 aa, Homo sapiens
GIIPAFGTANYAQKFQG

SEQ ID NO: 627 - protein, 18 aa, Homo sapiens
EQSPEYGYGGYPYEAMDV

SEQ ID NO: 628 - protein, 5 aa, Homo sapiens
DYAIS

SEQ ID NO: 629 - protein, 17 aa, Homo sapiens
GIIPAFGTANYAQKFQG

SEQ ID NO: 630 - protein, 18 aa, Homo sapiens
EQSPEYGYGGYPYEAMDV

SEQ ID NO: 631 - protein, 7 aa, Homo sapiens
GGTFRDY

SEQ ID NO: 632 - protein, 6 aa, Homo sapiens
IPAFGT

SEQ ID NO: 633 - protein, 18 aa, Homo sapiens
EQSPEYGYGGYPYEAMDV

SEQ ID NO: 634 - protein, 127 aa, Homo sapiens
QVQLVQSGAEVKKPGSSVKVSCKASGGTFRDYAISVWRQAPGQGLEWMGGIIPAFGTANYAQKFQGRVTITADESTSTAYMELSSLRSEDTAVYYCAREGSPEYGYGGYPYEAMDVWGQGTLVTVSS

SEQ ID NO: 635 - DNA, 381 bp, Homo sapiens
caggtgcagctggtgcagtcaggcgccgaagtgaagaaacccggctctagcgtgaaagtcagctgtaaagctagtggcggcacctttagagactacgctattagctgggtcagacaggccccaggtcagggcctggagtggatgggcggaattatccccgccttcggcaccgctaactacgctcagaaatttcagggtagagtgactatcaccgccgacgagtctactagcaccgcctatatggaactgtctagcctgagatcagaggacaccgccgtctactactgcgctagagagcagtcccccgagtacggctacggcggctacccctacgaggctatggacgtgtggggtcagggcaccctggtcaccgtgtctagc

SEQ ID NO: 636 - protein, 457 aa, Homo sapiens
QVQLVQSGAEVKKPGSSVKVSCKASGGTFRDYAISVWRQAPGQGLEWMGGIIPAFGTANYAQKFQGRVTITADESTSTAYMELSSLRSEDTAVYYCAREQSPEYGYGGYPYEAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 637 - DNA, 1371 bp, Homo sapiens
caggtgcagctggtgcagtcaggcgccgaagtgaagaaacccggctctagcgtgaaagtcagctgtaaagctagtggcggcacctttagagactacgctattagctgggtcagacaggccccaggtcagggcctggagtggatgggcggaattatccccgccttcggcaccgctaactacgctcagaaatttcagggtagagtgactatcaccgccgacgagtctactagcaccgcctatatggaactgtctagcctgagatcagaggacaccgccgtctactactgcgctagagagcagtcccccgagtacggctacggcggctacccctacgaggctatggacgtgtggggtcagggcaccctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 638 - protein, 11 aa, Homo sapiens
SGDNIPQHSVH

SEQ ID NO: 639 - protein, 7 aa, Homo sapiens
DDTERPS

SEQ ID NO: 640 - protein, 11 aa, Homo sapiens
SSWDSSMDSVV

SEQ ID NO: 641 - protein, 11 aa, Homo sapiens
SGDNIPQHSVH

SEQ ID NO: 642 - protein, 7 aa, Homo sapiens
DDTERPS

SEQ ID NO: 643 - protein, 11 aa, Homo sapiens
SSWDSSMDSVV

SEQ ID NO: 644 - protein, 7 aa, Homo sapiens
DNIPQHS

SEQ ID NO: 645 - skipped

SEQ ID NO: 646 - protein, 8 aa, Homo sapiens
WDSSMDSV

SEQ ID NO: 647 - protein, 108 aa, Homo sapiens; identical to SEQ ID NO: 361

SEQ ID NO: 648 - DNA, 324 bp, Homo sapiens; identical to SEQ ID NO: 180

SEQ ID NO: 649 - protein, 214 aa, Homo sapiens; identical to SEQ ID NO: 181

SEQ ID NO: 650 - DNA, 642 bp, Homo sapiens; identical to SEQ ID NO: 182

SEQ ID NO: 651 - protein, 10 aa, Homo sapiens
GGTFRDYAIS

SEQ ID NO: 652 - protein, 17 aa, Homo sapiens
GIIPAFGTANYAQKFQG

SEQ ID NO: 653 - protein, 18 aa, Homo sapiens
EQSPEYGYGGYPYEAMDV

SEQ ID NO: 654 - protein, 5 aa, Homo sapiens
DYAIS

SEQ ID NO: 655 - protein, 17 aa, Homo sapiens
GIIPAFGTANYAQKFQG

SEQ ID NO: 656 - protein, 18 aa, Homo sapiens
EQSPEYGYGGYPYEAMDV

SEQ ID NO: 657 - protein, 7 aa, Homo sapiens
GGTFRDY

SEQ ID NO: 658 - protein, 6 aa, Homo sapiens
IPAFGT

SEQ ID NO: 659 - protein, 18 aa, Homo sapiens
EQSPEYGYGGYPYEAMDV

SEQ ID NO: 660 - protein, 127 aa, Homo sapiens
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQSPEYGYGGYPYEAMDVWGQGTLVTVSS

SEQ ID NO: 661 - DNA, 381 bp, Homo sapiens
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacagagcccggaatacggttacggtggttacccgtatgaagctatggatgtttggggccaaggcaccctggtgactgttagctca

SEQ ID NO: 662 - protein, 457 aa, Homo sapiens
QVQLVQSGAEVKKPGSSVKVSCKASGGTFRDYAISWVRQAPGQGLEWMGGIIPAFGTANYAQKFQGRVTITADESTSTAYMELSSLRSEDTAVYYCAREQSPEYGYGGYPYEAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVQKSRWQQGNVFSCSVMHEALHNHYTQKSLSLSPGK

SEQ ID NO: 663 - DNA, 1371 bp, Homo sapiens
caggtgcaattggtgcagagcggtgccgaagtgaaaaaaccgggcagcagcgtgaaagttagctgcaaagcatccggagggacgtttcgtgactacgctatctcttgggtgcgccaggccccgggccagggcctcgagtggatgggcggtatcatcccggctttcggcactgcgaactacgcccagaaatttcagggccgggtgaccattaccgccgatgaaagcaccagcaccgcctatatggaactgagcagcctgcgcagcgaagatacggccgtgtattattgcgcgcgtgaacagagcccggaatacggttacggtggttacccgtatgaagctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 664 - protein, 11 aa, Homo sapiens
SGDNIPQHSVH

SEQ ID NO: 665 - protein, 7 aa, Homo sapiens
DDTERPS

SEQ ID NO: 666 - protein, 11 aa, Homo sapiens
SSWDSSMDSVV

SEQ ID NO: 667 - protein, 11 aa, Homo sapiens
SGDNIPQHSVH

SEQ ID NO: 668 - protein, 7 aa, Homo sapiens
DDTERPS

SEQ ID NO: 669 - protein, 11 aa, Homo sapiens
SSWDSSMDSVV

SEQ ID NO: 670 - protein, 7 aa, Homo sapiens
DNIPQHS

SEQ ID NO: 671 - skipped

SEQ ID NO: 672 - protein, 8 aa, Homo sapiens
WDSSMDSV

SEQ ID NO: 673 - protein, 108 aa, Homo sapiens
SYELTQPLSVSVALGQTARITCSGDNIPQHSVHWYQQKPGQAPVLVIYDDTERPSGIPERFSGSNSGNTATLTISRAQAGDEADYYCSSWDSSMDSVVFGQQTKLTVL

SEQ ID NO: 674 - DNA, 324 bp, Homo sapiens; identical to SEQ ID NO: 206

SEQ ID NO: 675 - protein, 213 aa, Homo sapiens
SYELTQPLSVSVALGQTARITCSGDNIPQHSVHWYQQKPGQAPVLVIYDDTERPSGIPERFSGSNSGNTATLTISRAQAGQEADYYCSSWDSSMDSWFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 676 - DNA, 642 bp, Homo sapiens; identical to SEQ ID NO: 208

SEQ ID NO: 677 - protein, 5 aa, Homo sapiens
SYGIH

SEQ ID NO: 678 - protein, 17 aa, Homo sapiens
VIGYDGSDKNYADSVKG

SEQ ID NO: 679 - protein, 9 aa, Homo sapiens
DQLGDAFDI

SEQ ID NO: 680 - protein, 118 aa, Homo sapiens
QVQLVESGGGVVQPGRSLRLSCAASGFTFSSYGIHWVRQAPGKGLEWVAVIGYDGSDKNYADSVKGRFTIFRDNSKNTLYLQMNSLRAEDTAVYYCARDQLGDAFDIWGQGTMVTVSS

SEQ ID NO: 681 - protein, 11 aa, Homo sapiens
KASQSVSSSLA

SEQ ID NO: 682 - protein, 7 aa, Homo sapiens
DASNRAT

SEQ ID NO: 683 - protein, 10 aa, Homo sapiens
QQRSNWPPYT

SEQ ID NO: 684 - protein, 109 aa, Homo sapiens
EIVLTQSPATLSLSPGERATLSCKASQSVSSSLAWYQQKPGQAPRLLIYDASNRATGIPARFSGSGSGTDFTLTISSLEPEDFAVYYCQQRSNWPPYTFGQGTKLEIKR

SEQ ID NO: 685 - protein, 5 aa, Homo sapiens
SYGIS

SEQ ID NO: 686 - protein, 17 aa, Homo sapiens
WISAYNGNTKYAQKLQG

SEQ ID NO: 687 - protein, 9 aa, Homo sapiens
DSAAHGMDV

SEQ ID NO: 688 - protein, 117 aa, Homo sapiens
QVQLVQSGGEVKKPGASVKVSCKTSGYTFTSYGISWVRQAPGQGLEWMGWISAYNGNTKYQKLQGRLTMTTDTSTTTAYMELRSLRSDDTAVYYCARDSAAHGMDVWGQGTTVTVSS

SEQ ID NO: 689 - protein, 11 aa, Homo sapiens
RASQGISSWLA

SEQ ID NO: 690 - protein, 7 aa, Homo sapiens
AASSLQS

SEQ ID NO: 691 - protein, 9 aa, Homo sapiens
QQYNSYPYT

SEQ ID NO: 692 - protein, 108 aa, Homo sapiens
DIQMTQSPSSLSASVGDRVTITCRASQGISSWLAWYQQKPEKAPKSLIYAASSLQSGVPSRFRGSGSGTDFTLTISSLQPEDFATYYCQQYNSYPYTFGQGTKLEIKR

SEQ ID NO: 693 - protein, 5 aa, Homo sapiens
SYGLS

SEQ ID NO: 694 - protein, 17 aa, Homo sapiens
WISPYNGNTHYAQKLQG

SEQ ID NO: 695 - protein, 9 aa, Homo sapiens
ASAAHGMDV

SEQ ID NO: 696 - protein, 118 aa, Homo sapiens
QVQLVQSGAEVKKPGASVKVSCKASGYTFTSYGLSWVRQAPGQGLEWMGWISPYNGNTHYAQKLQGRVTMTTDTSTSTADMDLRSLRSDDTAVYYCARASAAHGMDVWGQGTTVTVSS

SEQ ID NO: 697 - protein, 11 aa, Homo sapiens
RASQGISSWLA

SEQ ID NO: 698 - protein, 7 aa, Homo sapiens
AASSLQS

SEQ ID NO: 699 - protein, 9 aa, Homo sapiens
QQYNSYPYT

SEQ ID NO: 700 - protein, 108 aa, Homo sapiens
DIQMTQSPSSLSASVGDRVTITCRASQGISSWLAWYQQKPEKAPKSLIYAASSLQSGVPSRFSGSRSGTDFTLTISSLQPEDFATYYCQQYNSYPYTFGQGTKLEIKR

SEQ ID NO: 701 - protein, 5 aa, Homo sapiens
SYGLS

SEQ ID NO: 702 - protein, 17 aa, Homo sapiens
WISPYNGNTHYAQKLQG

SEQ ID NO: 703 - protein, 9 aa, Homo sapiens
DSAAHGMDV

SEQ ID NO: 704 - protein, 118 aa, Homo sapiens
QVQLVQSGAEVKKPGASVKVSCKASGYTFTSYGLSWVRQAPGQGLEWMGWISPYNGNTHYAQKLQGRVTMTTDTSTSTAYMDLRSLRSDDTAVYYCARDSAAHGMDVWGQGTTVTVSS

SEQ ID NO: 705 - protein, 11 aa, Homo sapiens
RASQGISSWLA

SEQ ID NO: 706 - protein, 7 aa, Homo sapiens
AASSLQS

SEQ ID NO: 707 - protein, 9 aa, Homo sapiens
QQYNSYPYT

SEQ ID NO: 708 - protein, 108 aa, Homo sapiens; identical to SEQ ID NO: 700

SEQ ID NO: 709 - protein, 5 aa, Homo sapiens
SYGLS

SEQ ID NO: 710 - protein, 17 aa, Homo sapiens
WISAYNGNTNYAQKLQG

SEQ ID NO: 711 - protein, 9 aa, Homo sapiens
DSAAHGMDV

SEQ ID NO: 712 - protein, 118 aa, Homo sapiens
QVQLVQSGAEVKKPGASVKVSCKASGYTFTSYGLSWVRQAPGQGLEWMGWISAYNGNTNYAQKLQGRVTMTTDTSTSTAYMDLRSLRSDDTAVYYCARDSAAHGMDVWGQGTTVTVSS

SEQ ID NO: 713 - protein, 11 aa, Homo sapiens
RASQGISSWLA

SEQ ID NO: 714 - protein, 7 aa, Homo sapiens
AASSLQS

SEQ ID NO: 715 - protein, 9 aa, Homo sapiens
QQYNSYPYT

SEQ ID NO: 716 - protein, 108 aa, Homo sapiens; identical to SEQ ID NO: 700

SEQ ID NO: 717 - protein, 5 aa, Homo sapiens
SYGIS

SEQ ID NO: 718 - protein, 17 aa, Homo sapiens
WISAYNGNTKYAQKLQG

SEQ ID NO: 719 - protein, 9 aa, Homo sapiens
DSAAHGMDV

SEQ ID NO: 720 - protein, 118 aa, Homo sapiens
QVQVVQSGAEVKKPGASVKVSCKTSGYTFTSYGISWVRQAPGQGLEWMGWISAYNGNTKYAQKLQGRLTMTTDTSTTTAYMELRSLRSDDTAVYYCARDSAAHGMDVWGQGTTVSVSS

SEQ ID NO: 721 - protein, 5 aa, Homo sapiens
NFVMS

SEQ ID NO: 722 - protein, 17 aa, Homo sapiens
GISGSGGNTDHADSVKG

SEQ ID NO: 723 - protein, 8 aa, Homo sapiens
DSGGLFDY

SEQ ID NO: 724 - protein, 117 aa, Homo sapiens
EVQLLESGGGLVQPGGSLRLSCAASGFTFSNFVMSWVRQAPGKGLEWVSGISGSGGNTDHADSVKGRFTISRDNSKNTVYLQMNSLRAEDTAVYYCAKDSGGLFDYWGLGTLVTVSS

SEQ ID NO: 725 - protein, 11 aa, Homo sapiens
RASQSVSSYLA

SEQ ID NO: 726 - protein, 7 aa, Homo sapiens
DASNRAT

SEQ ID NO: 727 - protein, 10 aa, Homo sapiens
QQRSNWPHLT

SEQ ID NO: 728 - protein, 109 aa, Homo sapiens
EIVLTQSPATLSLSPGERATLSCRASQSVSSYLAWYQQKPGQAPRLLIYDASNRATGIPARFSGSGSRTDFTLTISSLEPEDFAVYYCQQRSNWPHLTFGGGTKVEIKR

SEQ ID NO: 729 - protein, 5 aa, Homo sapiens
TYGMH

SEQ ID NO: 730 - protein, 17 aa, Homo sapiens
VISHDGSDKYYADSVKG

SEQ ID NO: 731 - protein, 10 aa, Homo sapiens
DQSIIETFDY

SEQ ID NO: 732 - protein, 119 aa, Homo sapiens
QVQLVESGGGVVQPGRSLRLSCAASGFTFSTYGMHWVRQAPGKGLEWVAVISHDGSDKYYADSVKGRFTISRDNSKNTLYLQMNSLRAEDTAVYYCARDQSIIETFDYWGQGTLVTVSS

SEQ ID NO: 733 - protein, 11 aa, Homo sapiens
RASQSVSSYLA

SEQ ID NO: 734 - protein, 7 aa, Homo sapiens
DASNRAT

SEQ ID NO: 735 - protein, 9 aa, Homo sapiens
QQRSNWGFT

SEQ ID NO: 736 - protein, 108 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWGFTFGPGTKVDIKR

SEQ ID NO: 737 - protein, 5 aa, Homo sapiens
SYGMH

SEQ ID NO: 738 - protein, 17 aa, Homo sapiens
VIWYDGSIKYYADSVKG

SEQ ID NO: 739 - protein, 9 aa, Homo sapiens
EGGRDAFDI

SEQ ID NO: 740 - protein, 118 aa, Homo sapiens
QVQLVESGGGVVQPGRSLRLSCAVSGFTFRSYGMHWVRQAPGKGLEWVAVIWYDGSIKYYADSVKGRFTISRDNSKNTLYLQMNSLRAEDTAVYFCAREGGRDAFDIWGQGTMVTVSS

SEQ ID NO: 741 - protein, 11 aa, Homo sapiens
RASQGISSALA

SEQ ID NO: 742 - protein, 7 aa, Homo sapiens
DASSLES

SEQ ID NO: 743 - protein, 9 aa, Homo sapiens
QQFNSYPHT

SEQ ID NO: 744 - protein, 108 aa, Homo sapiens
AVQLTQSPSSLSASVGDRVTITCRASQGISSALAWYQQKPGKAPKLLIYDASSLESGVPSRFSGSGSGTDFTLTISSLQPEDFATYCCQQFNSYPHTFGGGTKVEIKR

SEQ ID NO: 745 - protein, 5 aa, Homo sapiens
SYAMS

SEQ ID NO: 746 - protein, 17 aa, Homo sapiens
AISDSGGSTYYADSVKG

SEQ ID NO: 747 - protein, 9 aa, Homo sapiens
EIAVALFDY

SEQ ID NO: 748 - protein, 118 aa, Homo sapiens
EVQLLESGGGLVQPGGSLRLSCAASGFTFSSYAMSWVRQAPGKGLEWVSAISDSGGSTYYADSVKGRFTISRDNSKNTLYLQMNSLRAEDTAAYYCAKEIAVALFDYWGQGTLVTVSS

SEQ ID NO: 749 - protein, 11 aa, Homo sapiens
RASQSVSSYLA

SEQ ID NO: 750 - protein, 7 aa, Homo sapiens
DASNRAT

SEQ ID NO: 751 - protein, 10 aa, Homo sapiens
QQRSSWPPYT

SEQ ID NO: 752 - protein, 109 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSSWPPYTFGQGTKLEIKR

SEQ ID NO: 753 - protein, 127 aa, Homo sapiens; identical to SEQ ID NO: 192

SEQ ID NO: 754 - protein, 108 aa, Homo sapiens; identical to SEQ ID NO: 361

SEQ ID NO: 755 - protein, 127 aa, Homo sapiens; identical to SEQ ID NO: 452

SEQ ID NO: 756 - protein, 108 aa, Homo sapiens; identical to SEQ ID NO: 361

SEQ ID NO: 757 - protein, 310 aa, Homo sapiens
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISALPGYPECREMGETLPEKPANPTNPDEADKVGAENTITYSLLMHPDALEEPDDQNRI

SEQ ID NO: 758 - protein, 291 aa, Homo sapiens
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISANPTNPDEADKVGAENTITYSLLMHPDALEEPDDQNRI

SEQ ID NO: 759 - protein, 303 aa, Homo sapiens
MGILSFLPVLATESDWADCKSPQPWGHMLLWTAVLFLAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISALPGYPECREMGETLPEKPANPTNPDEADKVGAENTITYSLLMHPDALEEPDDQNRI

SEQ ID NO: 760 - protein, 309 aa, Homo sapiens
MGILSFLPVLATESDWADCKSPQPWGHMLLWTAVLFLAPVAGTPAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISALPGYPECREMGETLPEKPANPTNPDEADKVGAENTITYSLLMHPDALEEPDDQNRI

SEQ ID NO: 761 - protein, 290 aa, Homo sapiens
MGILSFLPVLATESDWADCKSPQPWGHMLLWTAVLFLAPVAGTPAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISANPTNPDEADKVGAENTITYSLLMHPDALEEPDDQNRI

SEQ ID NO: 762 - protein, 5 aa, Homo sapiens
DAWMN

SEQ ID NO: 763 - protein, 19 aa, Homo sapiens
EIRNKAKNHATYYAESVIG

SEQ ID NO: 764 - protein, 7 aa, Homo sapiens
GALGLDY

SEQ ID NO: 765 - protein, 11 aa, Homo sapiens
RASQEISGYLS

SEQ ID NO: 766 - protein, 7 aa, Homo sapiens
AASTLNS

SEQ ID NO: 767 - protein, 9 aa, Homo sapiens
LQYFSYPLT

SEQ ID NO: 768 - protein, 5 aa, Homo sapiens
DYYMN

SEQ ID NO: 769 - protein, 17 aa, Homo sapiens
LIGWDGGSTYYADSVKG

SEQ ID NO: 770 - protein, 9 aa, Homo sapiens
AYSGYELDY

SEQ ID NO: 771 - protein, 13 aa, Homo sapiens
SGSSSNIGNNAVN

SEQ ID NO: 772 - protein, 7 aa, Homo sapiens
DNNNRPS

SEQ ID NO: 773 - protein, 11 aa, Homo sapiens
AAWDDSLNASI

SEQ ID NO: 774 - protein, 5 aa, Homo sapiens
SYGMH

SEQ ID NO: 775 - protein, 17 aa, Homo sapiens
FTRYDGSNKYYADSVRG

SEQ ID NO: 776 - protein, 8 aa, Homo sapiens
ENIDAFDV

SEQ ID NO: 777 - protein, 13 aa, Homo sapiens
SGSSSNIGNNAVN

SEQ ID NO: 778 - protein, 7 aa, Homo sapiens
DNQQRPS

SEQ ID NO: 779 - protein, 9 aa, Homo sapiens
WDDRLFGPV

SEQ ID NO: 780 - protein, 5 aa, Homo sapiens
SYAMS

SEQ ID NO: 781 - protein, 17 aa, Homo sapiens
SISDSGAGRYYADSVEG

SEQ ID NO: 782 - protein, 13 aa, Homo sapiens
THDSGELLDAFDI

SEQ ID NO: 783 - protein, 13 aa, Homo sapiens
SGSSSNIGSNHVL

SEQ ID NO: 784 - protein, 7 aa, Homo sapiens
GNSNRPS

SEQ ID NO: 785 - protein, 11 aa, Homo sapiens
AAWDDSLNGWV

SEQ ID NO: 786 - protein, 5 aa, Homo sapiens
TYAMN

SEQ ID NO: 787 - protein, 17 aa, Homo sapiens
VISYDGSNKNYVDSVKG

SEQ ID NO: 788 - protein, 15 aa, Homo sapiens
NFDNSGYAIPDAFDI

SEQ ID NO: 789 - protein, 14 aa, Homo sapiens
TGSSSNIGAGYDVH

SEQ ID NO: 790 - protein, 7 aa, Homo sapiens
DNNSRPS

SEQ ID NO: 791 - protein, 11 aa, Homo sapiens
AAWDDSLGGPV

SEQ ID NO: 792 - protein, 5 aa, Homo sapiens
NAWMS

SEQ ID NO: 793 - protein, 17 aa, Homo sapiens
YISRDADITHYPASVKG

SEQ ID NO: 794 - protein, 12 aa, Homo sapiens
GFDYAGDDAFDI

SEQ ID NO: 795 - protein, 13 aa, Homo sapiens
SGSSSNIGSNAVN

SEQ ID NO: 796 - protein, 7 aa, Homo sapiens
GNSDRPS

SEQ ID NO: 797 - protein, 12 aa, Homo sapiens
AAWDDSLNGRWV

SEQ ID NO: 798 - protein, 5 aa, Homo sapiens
DYYMS

SEQ ID NO: 799 - protein, 17 aa, Homo sapiens
LIGHDGNNKYYLDSLEG

SEQ ID NO: 800 - protein, 10 aa, Homo sapiens
ATDSGYDLLY

SEQ ID NO: 801 - protein, 13 aa, Homo sapiens
SGSSSNIGNNAVN

SEQ ID NO: 802 - protein, 7 aa, Homo sapiens
YDDLLPS

SEQ ID NO: 803 - protein, 11 aa, Homo sapiens
TTWDDSLSGVV

SEQ ID NO: 804 - protein, 5 aa, Homo sapiens
DYYMS

SEQ ID NO: 805 - protein, 17 aa, Homo sapiens
AIGFSDDNTYYADSVKG

SEQ ID NO: 806 - protein, 8 aa, Homo sapiens
GDGSGWSF

SEQ ID NO: 807 - protein, 13 aa, Homo sapiens
SGSSSNIGNNAVN

SEQ ID NO: 808 - protein, 7 aa, Homo sapiens
DNNKRPS

SEQ ID NO: 809 - protein, 11 aa, Homo sapiens
ATWDDSLRGWV

SEQ ID NO: 810 - protein, 5 aa, Homo sapiens
NYGMH

SEQ ID NO: 811 - protein, 17 aa, Homo sapiens
VISYDGSNKYYADSVKG

SEQ ID NO: 812 - protein, 7 aa, Homo sapiens
WRDAFDI

SEQ ID NO: 813 - protein, 14 aa, Homo sapiens
TGSSSNIGAGYDVH

SEQ ID NO: 814 - protein, 7 aa, Homo sapiens
SDNQRPS

SEQ ID NO: 815 - protein, 12 aa, Homo sapiens
AAWDDSLSGSWV

SEQ ID NO: 816 - protein, 5 aa, Homo sapiens
TYGMH

SEQ ID NO: 817 - protein, 17 aa, Homo sapiens
VISYDGSNKYYADSVKG

SEQ ID NO: 818 - protein, 8 aa, Homo sapiens
ENFDAFDV

SEQ ID NO: 819 - protein, 14 aa, Homo sapiens
TGSSSNIGAGYDVH

SEQ ID NO: 820 - protein, 7 aa, Homo sapiens
SNSQRPS

SEQ ID NO: 821 - protein, 12 aa, Homo sapiens
AAWDDSLNGQVV

SEQ ID NO: 822 - protein, 5 aa, Homo sapiens
TYGMH

SEQ ID NO: 823 - protein, 17 aa, Homo sapiens
VIAYDGSKKDYADSVKG

SEQ ID NO: 824 - protein, 8 aa, Homo sapiens
EYRDAFDI

SEQ ID NO: 825 - protein, 14 aa, Homo sapiens
TGSSSNIGAGYDVH

SEQ ID NO: 826 - protein, 7 aa, Homo sapiens
GNSNRPS

SEQ ID NO: 827 - protein, 11 aa, Homo sapiens
AAWDDSVSGWM

SEQ ID NO: 828 - protein, 5 aa, Homo sapiens
SYGMH

SEQ ID NO: 829 - protein, 17 aa, Homo sapiens
VISYDGINKDYADSMKG

SEQ ID NO: 830 - protein, 8 aa, Homo sapiens
ERKDAFDI

SEQ ID NO: 831 - protein, 14 aa, Homo sapiens
TGSSSNIGAGYDVH

SEQ ID NO: 832 - protein, 7 aa, Homo sapiens
SNNQRPS

SEQ ID NO: 833 - protein, 11 aa, Homo sapiens
ATWDDSLNGLV

SEQ ID NO: 834 - protein, 5 aa, Homo sapiens
NYGMH

SEQ ID NO: 835 - protein, 17 aa, Homo sapiens
VISYDGSNRYYADSVKG

SEQ ID NO: 836 - protein, 8 aa, Homo sapiens
DRWNGMDV

SEQ ID NO: 837 - protein, 14 aa, Homo sapiens
SGSSSNIGAGYDVH

SEQ ID NO: 838 - protein, 7 aa, Homo sapiens
ANNQRPS

SEQ ID NO: 839 - protein, 12 aa, Homo sapiens
AAWDDSLNGPWV

SEQ ID NO: 840 - protein, 5 aa, Homo sapiens
SYGMH

SEQ ID NO: 841 - protein, 17 aa, Homo sapiens
VISYDGSDTAYADSVKG

SEQ ID NO: 842 - protein, 10 aa, Homo sapiens
DHSVIGAFDI

SEQ ID NO: 843 - protein, 13 aa, Homo sapiens
SGSSSNIGSNTVN

SEQ ID NO: 844 - protein, 7 aa, Homo sapiens
DNNKRPS

SEQ ID NO: 845 - protein, 10 aa, Homo sapiens
SSYAGSNNVV

SEQ ID NO: 846 - protein, 5 aa, Homo sapiens
SYGMH

SEQ ID NO: 847 - protein, 17 aa, Homo sapiens
VTSYDGNTKYYANSVKG

SEQ ID NO: 848 - protein, 10 aa, Homo sapiens
EDCGGDCFDY

SEQ ID NO: 849 - protein, 14 aa, Homo sapiens
TGSSSNIGAGYDVH

SEQ ID NO: 850 - protein, 7 aa, Homo sapiens
GNSNRPS

SEQ ID NO: 851 - protein, 11 aa, Homo sapiens
AAWDDSLNEGV

SEQ ID NO: 852 - protein, 5 aa, Homo sapiens
NYGMH

SEQ ID NO: 853 - protein, 17 aa, Homo sapiens
VISYDGSNKYYADSVKG

SEQ ID NO: 854 - protein, 9 aa, Homo sapiens
DQLGEAFDI

SEQ ID NO: 855 - protein, 14 aa, Homo sapiens
TGSSSNIGAGYDVH

SEQ ID NO: 856 - protein, 7 aa, Homo sapiens
DNNKRPS

SEQ ID NO: 857 - protein, 11 aa, Homo sapiens
ATWDDSLSGPV

SEQ ID NO: 858 - protein, 5 aa, Homo sapiens
DYGMS

SEQ ID NO: 859 - protein, 17 aa, Homo sapiens
AISGSGSSTYYADSVKG

SEQ ID NO: 860 - protein, 8 aa, Homo sapiens
GDIDYFDY

SEQ ID NO: 861 - protein, 14 aa, Homo sapiens
TGSSSNFGAGYDVH

SEQ ID NO: 862 - protein, 7 aa, Homo sapiens
ENNKRPS

SEQ ID NO: 863 - protein, 11 aa, Homo sapiens
AAWDDSLNGPV

SEQ ID NO: 864 - protein, 5 aa, Homo sapiens
SYGMH

SEQ ID NO: 865 - protein, 17 aa, Homo sapiens
VISYDGSNKYYADSVKG

SEQ ID NO: 866 - protein, 8 aa, Homo sapiens
ERRDAFDI

SEQ ID NO: 867 - protein, 14 aa, Homo sapiens
TGSSSNIGAGYDVH

SEQ ID NO: 868 - protein, 7 aa, Homo sapiens
SDNQRPS

SEQ ID NO: 869 - protein, 9 aa, Homo sapiens
ATWDSDTPV

SEQ ID NO: 870 - protein, 5 aa, Homo sapiens
SYGMH

SEQ ID NO: 871 - protein, 17 aa, Homo sapiens
VISYDGSNKYYADSVKG

SEQ ID NO: 872 - protein, 10 aa, Homo sapiens
DHSAAGYFDY

SEQ ID NO: 873 - protein, 13 aa, Homo sapiens
SGSSSNIGSNTVN

SEQ ID NO: 874 - protein, 7 aa, Homo sapiens
GNSIRPS

SEQ ID NO: 875 - protein, 11 aa, Homo sapiens
ASWDDSLSSPV

SEQ ID NO: 876 - protein, 5 aa, Homo sapiens
SYGMH

SEQ ID NO: 877 - protein, 17 aa, Homo sapiens
GISWDSAIIDYAGSVKG

SEQ ID NO: 878 - protein, 10 aa, Homo sapiens
DEAAAGAFDI

SEQ ID NO: 879 - protein, 14 aa, Homo sapiens
TGSSSNIGAGYDVH

SEQ ID NO: 880 - protein, 7 aa, Homo sapiens
GNTDRPS

SEQ ID NO: 881 - protein, 12 aa, Homo sapiens
AAWDDSLSGPVV

SEQ ID NO: 882 - protein, 5 aa, Homo sapiens
SYGIS

SEQ ID NO: 883 - protein, 17 aa, Homo sapiens
GISGSGGNTYYADSVKG

SEQ ID NO: 884 - protein, 12 aa, Homo sapiens
SVGAYANDAFDI

SEQ ID NO: 885 - protein, 14 aa, Homo sapiens
TGSSSNIGAGYDVH

SEQ ID NO: 886 - protein, 7 aa, Homo sapiens
GDTNRPS

SEQ ID NO: 887 - protein, 11 aa, Homo sapiens
AAWDDSLNGPV

SEQ ID NO: 888 - protein, 5 aa, Homo sapiens
SYGMH

SEQ ID NO: 889 - protein, 17 aa, Homo sapiens
VISYDGSNKYYADSVKG

SEQ ID NO: 890 - protein, 8 aa, Homo sapiens
ELYDAFDI

SEQ ID NO: 891 - protein, 14 aa, Homo sapiens
TGSSSNIGAGYDVH

SEQ ID NO: 892 - protein, 7 aa, Homo sapiens
ADDHRPS

SEQ ID NO: 893 - protein, 11 aa, Homo sapiens
ASWDDSQRAVI

SEQ ID NO: 894 - protein, 5 aa, Homo sapiens
NYGMH

SEQ ID NO: 895 - protein, 17 aa, Homo sapiens
VISYDGSNKYYADSVKG

SEQ ID NO: 896 - protein, 8 aa, Homo sapiens
EYKDAFDI

SEQ ID NO: 897 - protein, 13 aa, Homo sapiens
TGSSSNIGSNTVN

SEQ ID NO: 898 - protein, 7 aa, Homo sapiens
DNNKRPS

SEQ ID NO: 899 - protein, 9 aa, Homo sapiens
QAWGTGIRV

SEQ ID NO: 900 - protein, 5 aa, Homo sapiens
SYGMH

SEQ ID NO: 901 - protein, 16 aa, Homo sapiens
VISYDGSKYYADSVKG

SEQ ID NO: 902 - protein, 9 aa, Homo sapiens
EFGYIILDY

SEQ ID NO: 903 - protein, 13 aa, Homo sapiens
SGSSSNIGSNTVN

SEQ ID NO: 904 - protein, 7 aa, Homo sapiens
RDYERPS

SEQ ID NO: 905 - protein, 11 aa, Homo sapiens
MAWDDSLSGVV

SEQ ID NO: 906 - protein, 5 aa, Homo sapiens
NHGMH

SEQ ID NO: 907 - protein, 17 aa, Homo sapiens
VISYDGTNKYYADSVRG

SEQ ID NO: 908 - protein, 8 aa, Homo sapiens
ETWDAFDV

SEQ ID NO: 909 - protein, 13 aa, Homo sapiens
SGSSSNIGSNNAN

SEQ ID NO: 910 - protein, 7 aa, Homo sapiens
DNNKRPS

SEQ ID NO: 911 - protein, 9 aa, Homo sapiens
QAWDSSTVV

SEQ ID NO: 912 - protein, 115 aa, Homo sapiens
EVKFEESGGGLVQPGGSMKLSCAASGFTFSDAWMDWVRQGPEKGLEWVAEIRNKANNLATYYAESVKGRFTIPRDDSKSSVYLHMNSLRAEDTGIYYCYSPFAYWGQGTLVTVSA

SEQ ID NO: 913 - protein, 107 aa, Homo sapiens
DIQMTQSPSSLSASLGERVSLTCRASQEISGYLSWLQQKPDGTTRRLIYAASTLDSGVPKRFSGSWSGSDYSLTISSLESEDFADYYCLQYVSYPYTFGGGTKLEIK

SEQ ID NO: 914 - protein, 120 aa, Homo sapiens
QVQLVQSGAEVKKPGASVKVSCKASGYTFTNYWIHWVRQAPGQGLEWMGVIDPSDTYPNYNKKFKGRVTMTTDTSTSTAYMELRSLRSDDTAVYYCARNGDSDYYSGMDYWGQGTTVTVS

SEQ ID NO: 915 - protein, 107 aa, Homo sapiens
EIVLTQSPDFQSVTPKEKVTITCRTSQSIGTNIHWYQQKPDQSPKLLIKNVSESISGVPSRFSGSGSGTDFTLTINSLEAEDAATYYCQQSNTWPFTFGGGTKVEIK

SEQ ID NO: 916 - protein, 120 aa, Homo sapiens; identical to SEQ ID NO: 914

SEQ ID NO: 917 - protein, 107 aa, Homo sapiens
EIVLTQSPDFQSVTPKEKVTITCRTSQSIGTNIHWYQQKPDQSPKLLIKYVSESISGVPSRFSGSGSGTDFTLTINSLEAEDAATYYCQQSNTWPFTFGGGTKVEIK

SEQ ID NO: 918 - protein, 120 aa, Homo sapiens; identical to SEQ ID NO: 914

SEQ ID NO: 919 - protein, 106 aa, Homo sapiens
EIVLTQSPDFQSVTPKEKVTITCRTSQSIGTNIHWYQQKPDQSPKLLIKYASESISGVPSRFSGSGSGTDFTLTINSLEAEDAATYCQQSNTWPFTFGGGTKVEIK

SEQ ID NO: 920 - protein, 5 aa, Homo sapiens
NYWIH

SEQ ID NO: 921 - protein, 5 aa, Homo sapiens
DAWMD

SEQ ID NO: 922 - protein, 17 aa, Homo sapiens
VIDPSDTYPNYNKKFKG

SEQ ID NO: 923 - protein, 19 aa, Homo sapiens
EIRNKANNLATYYAESVKG

SEQ ID NO: 924 - protein, 12 aa, Homo sapiens
NGDSDYYSGMDY

SEQ ID NO: 925 - protein, 6 aa, Homo sapiens
YSPFAY

SEQ ID NO: 926 - protein, 11 aa, Homo sapiens
RTSQSIGTNIH

SEQ ID NO: 927 - protein, 11 aa, Homo sapiens
RASQEISGYLS

SEQ ID NO: 928 - protein, 7 aa, Homo sapiens
NVSESIS

SEQ ID NO: 929 - protein, 7 aa, Homo sapiens
YVSESIS

SEQ ID NO: 930 - protein, 7 aa, Homo sapiens
YASESIS

SEQ ID NO: 931 - protein, 7 aa, Homo sapiens
AASTLDS

SEQ ID NO: 932 - protein, 9 aa, Homo sapiens
QQSNTWPFT

SEQ ID NO: 933 - protein, 9 aa, Homo sapiens
LQYVSYPYT

(19) United States
(12) Patent Application Publication
(10) Pub. No.: US 2025/0270326 A1
(43) Pub. Date: Aug. 28, 2025

(54) METHODS OF TREATING RESPIRATORY DISORDERS

(71) Applicant: THE REGENTS OF THE UNIVERSITY OF CALIFORNIA, Oakland, CA (US)

(72) Inventors: Matthew F. KRUMMEL, San Francisco, CA (US); Alexis COMBES, San Francisco, CA (US); Tristan COURAU, San Francisco, CA (US); Nicholas KUHN, San Francisco, CA (US); Kenneth H. HU, San Francisco, CA (US); Arjun RAO, San Francisco, CA (US); Arja RAY, San Bruno, CA (US)

(21) Appl. No.: 18/259,268

(22) PCT Filed: Dec. 23, 2021
(86) PCT No.: PCT/US2021/065148

§ 371 Date: Jun. 23, 2023

Related U.S. Application Data
(60) Provisional application No. 63/130,403, filed on Dec. 23, 2020.

(51) Int. Cl. C07K 16/28; A61K 39/00; A61P 31/14

(52) U.S. Cl. CPC C07K 16/283; A61P 31/14; A61K 2039/505

(57) ABSTRACT

The application discloses methods of treating and/or preventing acute respiratory disorder in a subject in need thereof. The application also discloses a method of treating a viral infection, such as Coronaviridae infection by administering to a subject infected with a virus from Coronaviridae with an agonist or antagonist of CD32. Antibodies and antibody binding fragments thereof are disclosed as treatments, and, in some embodiments, the antibody treatment is an antibody against an epitope in CD32b.

### CROSS-REFERENCE TO RELATED APPLICATIONS

The present application is a PCT Application filed under 35 USC § 361 and claims priority to U.S. Provisional Patent Application No. 63/130,403, filed Dec. 23, 2020, the contents of which are hereby incorporated by reference in its entirety.

### STATEMENT REGARDING FEDERALLY SPONSORED RESEARCH

This invention was made with government support under Grant No. R01 AI52116 awarded by the National Institutes of Health. The government has certain rights in the invention.

### FIELD OF INVENTION

The disclosure relates to the treatment of subjects infected with Coronavirus to lessen the severity of acute respiratory disorder associated with viral infection. The treatment comprises an agonist or antagonist of CD32, and, in some embodiments, the treatment is an antagonist of CD32a or CD32b. In some embodiments, the treatment is a therapeutically effective amount of an antagonist of CD32b that is selective for CD32b and not CD32a.

### BACKGROUND

Viral infections, such as respiratory viral infections, have been significant threats to human health and lives for centuries. Notorious episodes include infections caused by influenza strains, infections caused by respiratory syncytial virus, and sever acute respiratory syndrome (SARS) caused by coronavirus. These include the global influenza pandemic of 1918, which killed approximately 20-40 million people worldwide, as well as the ongoing global coronavirus pandemic caused by SARS-COV-2.

### SUMMARY OF EMBODIMENTS

In one aspect, the disclosure relates to a method of abrogating severity of a viral infection that induces interferon in a subject in need thereof comprising administering to the subject in need thereof a pharmaceutical composition comprising: (a) a therapeutically effective amount of a CD32b antagonist or agonist; and (ii) and one or a plurality of pharmaceutically acceptable carriers. In another aspect, the disclosure relates to a method of treating Coronaviridae infection in a subject in need thereof comprising administering to the subject in need thereof a therapeutically effective amount of a pharmaceutical composition comprising: (i) a CD32b antagonist; and (ii) one or a plurality of pharmaceutically acceptable carriers. In yet another aspect, the disclosure relates to a method of treating Coronaviridae infection in a subject in need thereof, said method comprising administering to the subject in need thereof a therapeutically effective amount of a CD32b antagonist or agonist and one or a plurality of pharmaceutically acceptable carriers. In a further aspect, the disclosure relates to a method of preventing acute respiratory distress syndrome in a subject in need thereof comprising administering to the subject in need thereof a pharmaceutical composition comprising: (i) a therapeutically effective amount of a CD32b antagonist or agonist; and (ii) one or a plurality of pharmaceutically acceptable carriers. In some embodiments, the acute respiratory distress syndrome is caused by a viral infection of the subject.

In some embodiments, the viral infection is a Coronaviridae viral infection. In some embodiments, the viral infection is a coronavirus infection. In some embodiments, the viral infection is a human coronavirus 229E (HCoV-229E) infection, human coronavirus OC43 (HCoV-OC43) infection, severe acute respiratory syndrome coronavirus (SARS-COV), human coronavirus NL63 (HCoV-NL63), human coronavirus HKUI, Middle East respiratory syndrome-related coronavirus (MERS-COV), or severe acute respiratory syndrome coronavirus 2 (SARS-COV-2) infection. In some embodiments, the viral infection is a SARS-COV-2 infection. In some embodiments, the subject in need thereof is diagnosed with or suspected of having a SARS-COV-2 infection. In some embodiments, the therapeutically effective amount of the CD32b antagonist or agonist is from about 0.1 mg to about 15,000 mg. In some embodiments, the therapeutically effective amount of the CD32b antagonist or agonist is from about 1 mg to about 1,000 mg.

In some embodiments, the CD32b antagonist is an anti-CD32b antibody or antigen-binding fragment thereof. In some embodiments, the anti-CD32b antibody or antigen-binding fragment thereof is a humanized monoclonal antibody or antigen-binding fragment thereof. In some embodiments, the humanized monoclonal antibody or antigen-binding fragment thereof comprises at least one CDR identified in Table 1 or at least one CDR comprising at least about 70% sequence identity to a CDR sequence identified in Table 1. In some embodiments, the humanized monoclonal antibody or antigen binding fragment thereof comprises two or three CDRs identified in Table 1, or two or three CDRs comprising at least about 70% sequence identity to a CDR sequence identified in Table 1. In some embodiments, the humanized monoclonal antibody or antigen-binding fragment thereof comprises: (a) a VH region comprising at least about 70% sequence identity to a variable heavy sequence identified in Table 1; and (b) a VL region comprising at least about 70% sequence identity to a variable light chain identified in Table 1. In some embodiments, the antigen-binding fragment is a Fc fragment or a Fv fragment. In some embodiments, the humanized monoclonal antibody or antigen-binding fragment thereof further comprises a heavy chain constant region. In some embodiments, the humanized monoclonal antibody or antigen-binding fragment thereof further comprises a light chain constant region. In some embodiments, the humanized monoclonal antibody or antigen-binding fragment thereof binds specifically to CD32b, or an immunogenic fragment or epitope thereof. In some embodiments, the CD32b comprises at least about 70% sequence identity to SEQ ID NO: 757, SEQ ID NO: 758, SEQ ID NO: 759, SEQ ID NO: 760 or SEQ ID NO: 761. In some embodiments, the CD32b comprises the amino acid sequence of SEQ ID NO: 757, SEQ ID NO: 758, SEQ ID NO: 759, SEQ ID NO: 760 or SEQ ID NO: 761. In some embodiments, the antibody or antibody-binding fragment is multivalent IgG1 molecule or an IgG3 molecule.

In some embodiments, the CD32b antagonist or is administered intravenously, intramuscularly, topically intradermally, transmucosally, subcutaneously, sublingually, orally, intravaginally, intraocularly, intranasally, intrarectally, gastrointesinally, intraductally, inthecally, subdurally, exradurally, intraventricularly, intraarticuarly, intraperitoneally, or into the pleural cavity. In some embodiments, the disclosed methods further comprise an active agent that reduces total plasma cell or B cell counts. In some embodiments, the active agent is a monoclonal antibody that binds CD20. In some embodiments, the active agent is Rituxan.

In another aspect, the disclosure relates to a method of restoring a neutrophil population of cells within a cell population of peripheral blood mononuclear cells (PBMCs) comprising contacting a CD32b antagonist or agonist to one or a plurality of cells that express CD32b. In a further aspect, the disclosure relates to a method of restoring a neutrophil population of cells within a cell population of peripheral blood mononuclear cells (PBMCs) comprising contacting a CD32b antagonist or agonist to one or a plurality of cells that express CD32b.

In some embodiments, the CD32b antagonist is an anti-CD32b antibody or antigen-binding fragment thereof. In some embodiments, the anti-CD32b antibody or antigen-binding fragment thereof is a humanized monoclonal antibody or antigen-binding fragment thereof. In some embodiments, the humanized monoclonal antibody or antigen-binding fragment thereof comprises at least one CDR identified in Table 1 or at least one CDR comprising at least about 70% sequence identity to a CDR sequence identified in Table 1. In some embodiments, the humanized monoclonal antibody or antigen binding fragment thereof comprises two or three CDRs identified in Table 1, or two or three CDRs comprising at least about 70% sequence identity to a CDR sequence identified in Table 1. In some embodiments, the humanized monoclonal antibody or antigen-binding fragment thereof comprises: (a) a VH region comprising at least about 70% sequence identity to a variable heavy sequence identified in Table 1; and (b) a VL region comprising at least about 70% sequence identity to a variable light chain identified in Table 1. In some embodiments, the antigen-binding fragment is a Fc fragment or a Fv fragment. In some embodiments, the humanized monoclonal antibody or antigen-binding fragment thereof further comprises a heavy chain constant region. In some embodiments, the humanized monoclonal antibody or antigen-binding fragment thereof further comprises a light chain constant region. In some embodiments, the humanized monoclonal antibody or antigen-binding fragment thereof binds specifically to CD32b, or an immunogenic fragment or epitope thereof. In some embodiments, the CD32b comprises at least about 70% sequence identity to SEQ ID NO: 757, SEQ ID NO: 758, SEQ ID NO: 759, SEQ ID NO: 760 or SEQ ID NO: 761. In some embodiments, the CD32b comprises the amino acid sequence of SEQ ID NO: 757, SEQ ID NO: 758, SEQ ID NO: 759, SEQ ID NO: 760 or SEQ ID NO: 761.

In further aspects, the disclosure relates to a method of inducing secretion of interferon-alpha (IFN-α) in a subject in need thereof comprising administering to the subject in need thereof a pharmaceutical composition comprising: (i) a therapeutically effective amount of a CD32b antagonist or agonist; and (ii) one or a plurality of pharmaceutically acceptable carriers. In some embodiments, the subject in need thereof is diagnosed with or suspected of having a SARS-COV-2 infection. In some embodiments, the therapeutically effective amount of the CD32b antagonist or agonist is from about 0.1 mg to about 15,000 mg. In some embodiments, the CD32b antagonist is an anti-CD32b antibody or antigen-binding fragment thereof. In some embodiments, the anti-CD32b antibody or antigen-binding fragment thereof is a humanized monoclonal antibody or antigen-binding fragment thereof having one or more characteristics described above and elsewhere in the disclosure.

The disclosure further relates to method of treating acute lung injury in a subject in need thereof administering to the subject in need thereof a pharmaceutical composition comprising: (i) a therapeutically effective amount of a CD32b antagonist or agonist; and (ii) one or a plurality of pharmaceutically acceptable carriers. The disclosure further relates to a method of treating an autoinflammatory disease or disorder in a subject in need thereof comprising administering to the subject in need thereof a pharmaceutical composition comprising: (i) a therapeutically effective amount of a CD32b antagonist or agonist; and (ii) one or a plurality of pharmaceutically acceptable carriers. The disclosure additionally relates to a method of treating a respiratory tract infection in a subject in need thereof comprising administering to the subject in need thereof a pharmaceutical composition comprising: (i) a therapeutically effective amount of a CD32b antagonist or agonist; and (ii) one or a plurality of pharmaceutically acceptable carriers. In some embodiments, the subject in need thereof is diagnosed with or suspected of having a SARS-COV-2 infection. In some embodiments, the therapeutically effective amount of the CD32b antagonist or agonist is from about 0.1 mg to about 15,000 mg. In some embodiments, the CD32b antagonist is an anti-CD32b antibody or antigen-binding fragment thereof. In some embodiments, the anti-CD32b antibody or antigen-binding fragment thereof is a humanized monoclonal antibody or antigen-binding fragment thereof having one or more characteristics described above and elsewhere in the disclosure.

### BRIEF DESCRIPTION OF DRAWINGS

FIG. 1A-1G depict that severe COVID-19 disease is characterized by the lack of IFN-responsive neutrophils. FIG. 1A: Gender, SARS-COV-2 status and disease severity in patients and control individuals (left) and description of study design (right). FIG. 1B: UMAP visualization of 116,517 cells merged from the entire cohort with specific populations overlaid (left), and frequencies of these populations across control, mild/moderate (M/M) and severe individuals (right). FIG. 1C: UMAP visualization of neutrophil subsets. FIG. 1D and FIG. 1E: Overlay of SARS-COV-2 status and disease severity, respectively, on the neutrophil UMAP. FIG. 1F: Frequency of ISG neutrophils among all neutrophils across SARS-COV-2 status and disease severity (CTRL, n=14; NEG M/M, n=6; NEG severe, n=5; POS M/M, n=11, POS severe n=10). FIG. 1G: Score of ISG signature across neutrophil subtypes and disease severity in SARS-COV-2 positive patients. Statistical significance was assessed using a two-way ANOVA test with multiple comparisons for panel a and e, and using a two-sided Wilcoxon test for panel f. *p-value<0.05; p-value<0.01; ***p-value<0.001; p-value<0.0001. Boxplot center, median; box limits, 25^{th} and 75^{th} percentile; whiskers, min. and max. data point.

FIG. 2A-2H depict that severe COVID-19 disease is defined by the lack of a concerted IFN-response across peripheral blood immune cells. FIG. 2A: Frequencies of MPC subsets among all MPC across mild/moderate (M/M, n=6 NEG, n=11 POS) and severe (n=5 NEG, n=10 POS) individuals. FIG. 2B: Scatter plot between neutrophil and monocyte ISG positive subsets patient by patient (M/M, n=11; severe, n=10; COVID-, n=11). FIG. 2C: Violin plot of ISG signature on all T cells (top) and all B/Plasma cells (bottom) across SARS-COV-2 status and disease severity. Statistical significance was assessed using a two-sided Wilcoxon test. FIG. 2D: Correlation matrix using Spearman rank correlation between the most and the least correlated cell subsets to the Neutrophils ISG positive cells (data include all SARS-COV-2 negative and positive patients). FIG. 2E: ISG signature score in all platelets across SARS-COV-2 status and disease severity. FIG. 2F-2H: 3D PhEMD embedding of all patients, colored by de novo patient clusters A-H (FIG. 2), SARS-COV-2 status (FIG. 2G), and disease severity (FIG. 2H). Statistical significance was assessed using two-tailed Spearman's rank correlation (b) and Kruskal Wallis test with multiple comparisons (a), and two-sided Wilcoxon rank sum test for panels c and e. *p-value<0.05; **p-value<0.01; ***p-value<0.001; ****p-value<0.0001. Boxplot center, median; box limits, 25^{th} and 75^{th} percentile; whiskers, min. and max. data point.

FIG. 3A-3H depict neutralization of ISG induction by antibodies from severe COVID-19 patients. FIG. 3A: Measurement of serum IFNα concentration from SARS-COV-2 negative and positive M/M (n=17) or severe (n=15) patients by ELISA. Patients 1055 and 1060 are highlighted in red and their Monocytes ISG frequency from FIG. 2C is noted as well as the median for mild COVID-19 mild/moderate patients. Boxplot center, median; box limits, 25^{th} and 75^{th} percentile; whiskers, 1.5× interquartile range (IQR). FIG. 3B: Measurement of anti-SARS-COV-2 antibody levels in serum from patients by Luminex assay (M/M: Mild/Moderate). Boxplot center, median; box limits, 25^{th} and 75^{th} percentile; whiskers, min. and max. data point. FIG. 3C: Scatter plots showing viral load versus levels of antibody binding SARS-COV-2 Nucleocapsid for patients in the cohort with severity overlaid. Antibody levels are shown as arbitrary units of MFI from Luminex assay while viral load is represented by an inverse CT number from QRT-PCR with target amplification of the SARS-COV2 Nucleocapsid sequence. Correlation coefficient and significance calculated using Spearman's method. Patients for which data was unavailable were excluded (M/M, n=9; severe, n=7 patients). FIG. 3D: Scatterplot for SARS-COV2 Full Spike protein antibody titers relative to days post symptom onset. Patients for which data was unavailable were excluded (M/M, n=14; severe, n=8 patients). FIG. 3E: Contour plots and histograms of CD14 and IFITM3 expression by monocytes from healthy PBMC cultured with IFNα and serum from either heathy donor, mild/moderate or severe SARS-COV-2 positive patient. FIG. 3F: Contour plots and histograms of CD14 and IFITM3 expression by monocytes after pre-treating Mild/Moderate (light yellow) or Severe (pink) sera with protein A/G prior to incubation with PBMC to deplete IgG. FIG. 3G: Boxplots of IFITM3 induction in CD14 monocytes (left; ctrl, n=5; M/M, n=21; severe, n=14; M/M depleted, n=11; severe depleted, n=10) and classical to intermediate monocytes ratio (right; ctrl, n=4; M/M, n=24; severe, n=7; M/M depleted, n=11; severe depleted, n=7) from 2 different experiment and 2 different healthy donors. FIG. 3H: Left: Contour plots and histograms of IFITM3 expression by pooled CD3+/CD19+ lymphocytes from healthy PBMC cultured with IFNα and serum from heathy donor, mild/moderate or severe SARS-CoV-2 positive patients. Light gray indicate respectively Mild/moderate and Severe sera pre-treated with protein A/G. Right: Box plot of IFITM3 induction in lymphocytes. Differences in FIG. 3G and FIG. 3H were calculated using a two-way ANOVA test with multiple comparisons. *p-value<0.05; **p-value<0.01; ***p-value<0.001; ****p-value<0.0001; ns: non-significant. For b/g/h boxplot center, median; box limits, 25^{th} and 75^{th} percentile; whiskers, min. and max. data point.

FIG. 4A-4E depict that IgG-mediated neutralization of ISG induction by Severe COVID-19 patients sera occurs through binding of their Fc to CD32. FIG. 4A: Contour plots and histograms of CD14 and IFITM3 expression by monocytes from healthy PBMC cultured with IFNα and serum from either heathy donor, mild/moderate or severe SARS-COV-2 positive patient, in the presence or not of anti-CD16/CD32/CD64 antibodies to block Fc receptors. FIG. 4B and FIG. 4C: CD14/IFITM3 contour plot and histograms (FIG. 4B) and boxplots presenting fold changes of IFITM3 expression (FIG. 4C) on CD14 monocytes after culturing healthy PBMCs+/−IFNα (1 pg/μl)+/−5 or 10 μg/ml of plate-coated isotype control, anti-CD16, anti-CD32 or anti-CD64 antibodies alone or in combination to cross-link and activate Fc receptors. Panel c presents the results of 2 independent experiments and 2 different cell donors, including two antibody quantities for one of the donors (n=3 experiments). Data is plotted as mean±SD. FIG. 4D: Neutralization assay as presented in panel a, with the sole addition of anti-CD32 blocking antibodies. FIG. 4E: Boxplots showing fold changes of IFITM3 expression for experiments presented in panel a (left graph, 5 independent experiments on 3 different cell donors) and panel d (right graph, 1 experiment on 2 different cell donors). Differences in FIG. 4C and FIG. 4E were calculated using a two-way ANOVA test with multiple comparisons. *p-value<0.05; **p-value<0.01; ****p-value<0.0001. Boxplot center, median; box limits, 25^{th} and 75^{th} percentile; whiskers, min. and max. data point. *p-value<0.05; **p-value<0.01; ***p-value<0.001; p-value<0.0001.

FIG. 5A: Patient symptoms plot: symptom at day of sampling (first day of admission to the hospital) is represented in black, while symptom based on the entire course of hospitalization is in gray. We categorized patient into mild/moderate or severe cases based on all the entire course of hospitalization (gray). FIG. 5B: Quantification of the batch effect using neighbor diversity score in the global object UMAP before (left) and after (middle) batch correction, along with the neutrophil (right) UMAP plot, as in FIG. 1B and FIG. 1C, using the diversity in neighborhood method. FIG. 5C: Dotplot representation of landmark genes expressed by global populations in FIG. 1B. FIG. 5D: Spearman's correlation comparison between disease severity and population frequencies calculated from 10×scRNAseq analyses (10×) or complete blood cell counts (CBC). Patients for which CBC counts were unavailable were excluded. Significance was calculated using Spearman's method. *p-value<0.05; **p-value<0.05; ***p-value<0.005 (n=29). FIG. 5E: Frequency of the global populations in FIG. 1B among all cells across SARS-COV-2 status (control, n=14; NEG, n=11; POS, n=21).

FIG. 6A: Dotplot representation of top differentially-expressed-genes (DEG) between neutrophil subsets. FIG. 6B: Frequencies of neutrophil subsets among all neutrophils across control (n=14), SARS-COV-2 negative (n=11) and SARS-COV-2 positive (n=21) individuals. FIG. 6C: Frequency of the LCN2, S100A12, RIBO, NEAT1, GOS2 and SLPI neutrophils among all neutrophils across SARS-COV-2 status and disease severity (NEG M/M, n=6; NEG severe, n=5; POS M/M, n=11, POS severe n=10). FIG. 6D: Pseudotime trajectory of neutrophil subsets. FIG. 6E: Frequencies of the neutrophil subsets among all neutrophils at later stages of pseudotime trajectories across control (n=14), mild/moderate (n=17) and severe (n=15) individuals. FIG. 6F and FIG. 6G: Frequencies of the neutrophil subsets among all neutrophils across control (n=14), mild/moderate (M/M, n=17) and severe (n=15) individuals at the overall start/late states of the trajectories (FIG. 6F) or at specific early stages of the pseudotime (FIG. 6G). FIG. 6H-6K: Volcano plots showing DEG (FIG. 6H and FIG. 6J) and bar plots showing GO term enrichment from these DEG (FIG. 6I and FIG. 6K) between all neutrophils from either SARS-COV-2 positive vs negative patients (FIG. 6H and FIG. 6I) or mild/moderate vs severe patients (FIG. 6J and FIG. 6K). FIG. 6L-6P: Scores of ISG signature (FIG. 6L-6N) and neutrophil degranulation (FIG. 6O and FIG. 6P) in either all neutrophils across control, mild/moderate an severe patients (FIG. 6L and FIG. 6O), all neutrophils across SARS-COV-2 status and disease severity (FIG. 6M and FIG. 6P) or specific neutrophil subtypes across severity in either all patients (FIG. 6M) or only SARS-CoV-2 negative patients (FIG. 6N). Statistical significance was assessed using a two-way ANOVA test with multiple comparisons for panels c, e and g, and using a two-tailed Wilcoxon test for panel 1. *p-value<0.05; **p-value<0.01; ***p-value<0.001; ****p-value<0.0001. Boxplot center, median; box limits, 25^{th} and 75^{th} percentile; whiskers, min. and max. data point.

FIG. 7A: Dotplot representation of the top differentially-expressed-genes (DEG) between clusters identified in blood mononuclear phagocytic cell (MPC) subsets. FIG. 7B: UMAP visualization of the 19,289 MPC isolated from the entire dataset (left) and split by SARS-COV-2 status (right). FIG. 7C: Quantification of the batch effect before and after batch correction using neighbor diversity score in the mononuclear phagocytic cells (MPC) object from UMAP plot in (FIG. 7B), using the diversity in neighborhood method. FIG. 7D: Violin plot of number of unique genes (bottom) and number of unique molecules (top) detected from Single cell sequencing for each cluster identified in the MPC dataset. FIG. 7E: Overlay of previously described blood mononuclear phagocytic cell signature from healthy individual on MPC from UMAP plot in (FIG. 7B). FIG. 7F: Violin plots of canonical genes previously described as expressed by blood MPC for each for each cluster identified in the MPC dataset.

FIG. 8A: Frequencies of the MPC subsets among all MPC across control (n=14), SARS-CoV-2 negative (n=11) and SARS-COV-2 positive (n=21) individuals. FIG. 8B: UMAP visualization of the 19,289 MPC colored (left) and split by (right) by disease severity. FIG. 8C: Frequencies of the classical monocytes, cycling monocytes, non-classical monocytes and CIQ+ non classical monocytes among all MPC across SARS-COV-2 negative (M/M, n=6; severe, n=5) and SARS-COV-2 positive (M/M, n=11; severe, n=10) individuals split it by disease severity. FIG. 8D: Overlay of previously described glycolytic and oxidative phosphorylation gene signature on mononuclear phagocytic cells (MPC) from UMAP plot in FIG. 7B. FIG. 8E: Volcano plot showing results of differential gene expression (DGE) analysis performed on all MPC between mild/moderate (right) and severe (left) patients. FIG. 8F: Correlation matrix using Spearman Rank Correlation between the frequency of all neutrophils and monocytes subtypes in all SARS-CoV-2 negative (n=11) and SARS-COV-2 positive patients (n=21). FIG. 8G: Scatter plot between neutrophil and CD4 T cell ISG positive subsets patient by patient (M/M, n=11; severe, n=10; COVID-, n=11). Statistical significance was assessed using a two-way ANOVA test with multiple comparisons. *p-value<0.05; **p-value<0.01; ***p-value<0.001; ****p-value<0.0001. Boxplot center, median; box limits, 25^{th} and 75^{th} percentile; whiskers, min. and max. data point.

FIG. 9A: Dotplot representation of the top DEG between clusters identified in the T and NK cell subset. FIG. 9B: UMAP visualization of 16,708 T and NK cells in the entire dataset showing various subsets, colored distinctly by their identity. FIG. 9C: Overlay of the above UMAP of all T and NK cells, colored by disease severity underlining the lack of batch effects while merging the datasets from all patients. FIG. 9D: Abundance of the Interferon-stimulated-gene (ISG)+subset among all T and NK cells in healthy donors (n=13), SARS-COV-2 negative (n=9) and SARS-COV-2 positive (n=15) patients (top) and in healthy donors and patients with mild/moderate (M/M, n=14) and severe disease (bottom, n=9). FIG. 9E: ISG signature score between healthy controls, SARS-COV-2 negative and SARS-COV-2 positive patients. FIG. 9F: Dotplot representation of the top DEG between clusters identified in the B and plasma cell subset. FIG. 9G: UMAP visualization of 4,380 B and plasma cells isolated from the entire dataset showing various subsets, colored distinctly by their identity. FIG. 9H: Violin plots of canonical genes previously described as expressed by B and plasma cells for each identified cluster. FIG. 9I: Frequencies of the identified clusters among all B and plasma cells in healthy donors (n=14) and patients with M/M (n=17) and severe disease (n=15). Differences in FIG. 9D and FIG. 9E were calculated using Kruskal-Wallis test. *p-value<0.05 and **p-value<0.001. Differences in FIG. 9I were calculated using a two-way ANOVA test with multiple comparisons. *p-value<0.05 and **p-value<0.0001. ns, non-significant. Boxplot center, median; box limits, 25^{th} and 75^{th} percentile; whiskers, min. and max. data point.

FIG. 10A: Dotplot representation of the top DEG between clusters identified in the platelet subset. FIG. 10B: UMAP visualization of 16,903 platelets isolated from the entire dataset showing various subsets, colored distinctly by their identity. FIG. 10C: Frequencies of the identified clusters among all platelets in controls (n=14) and all patients with mild/moderate (M/M, n=17) and severe disease (n=15). FIG. 10D: UMAP visualization of all platelets colored by BCL2L1 (top) and violin plot of BCL2L1 expression level across all identified platelet subsets. FIG. 10E: Violin plots of genes identifying young, reticulated platelets (1) in the platelet dataset. FIG. 10F: UMAP visualization of all platelets with overlay of Pseudotime trajectory. FIG. 10G: Violin plots of the relative pseudotime of each platelet cell subset present in FIG. 3B. FIG. 10H: Violin plot of the relative Pseudotime of all platelets split by healthy donors, mild/moderate and severe patients. FIG. 10I: UMAP visualization of all platelets colored by ISG score. Differences in FIG. 10C were calculated using a two-way ANOVA test with multiple comparisons. *p-value<0.05; **p-value<0.01; ***p-value<0.001; ****p-value<0.0001; ns: non-significant. Boxplot center, median; box limits, 25^{th} and 75^{th} percentile; whiskers, min. and max. data point.

FIG. 11A: Outline of “Platelet First” assessment to identify platelet aggregates in entire whole blood scRNA-seq data set. UMAP visualization of the 52,757 putative platelet aggregates with specific populations overlaid. FIG. 11B: Dotplot representation of the top DEG between clusters identified in the “Platelet First” object. In this object no doublet removal filtering step was applied to include all heterotypic cell-cell aggregates (Step 1). This was followed by retaining all cells with >1 platelet-specific transcripts PF4 or PPBP (Step 2). Step 2 guaranteed analysis of cell events and aggregates containing platelets. Identically to our original data set in FIG. 1B, integration of data was done using Harmony (Step 3), and the “Platelet First” object was then analyzed using the Seurat v3 pipeline (Step 4). FIG. 11C: Violin plots of the percentage of mitochondrial and ribosomal genes within clusters identified in the “Platelet First” object. FIG. 11D: Inter-sample doublet rates in inferred platelet-involved heterotypic doublets show that platelet aggregates occur in vivo. DBL, doublet, n=5 libraries. SNG, singlet, n=5 libraries. FIG. 11E: Bottom: Scatter plot of cell type frequency within merged object of entire cohort shown in FIG. 1B (x-axis) versus same cell type frequency within “Platelet First” object (y-axis). The identity line x=y is drawn as a reference. Each dot represents a control (n=14) or SARS-COV-2 positive patient sample and are color-coded by disease severity (M/M, n=11; severe, n=10). Pearson r correlation coefficient and two-tailed p value are shown for each cell type. Top: Box plots of y x-ratio for each healthy control or patient sample, separated by disease severity. FIG. 11F: Cell fraction histograms representing bin-wise mean of relative frequency (i.e., cell fraction) of each immune cell subtype for all patients in a given group, colored as described in FIG. 2F. Differences in FIG. 11D were calculated using a one-sided Student's t test. *p-value<0.05 and **p-value<0.01. Differences in FIG. 11E were calculated using a two-way ANOVA test with multiple comparisons. *p-value<0.05; **p-value<0.01; ***p-value<0.001; ****p-value<0.0001; ns: non-significant. Boxplot center, median; box limits, 25^{th} and 75^{th} percentile; whiskers, min. and max. data point.

FIG. 12A: Matrix of Spearman correlation coefficients between all subtype frequencies (out of major cell types, e.g. Neut ISG out of all Neutrophils) obtained from scRNA-Seq versus patient metadata, viral load, Ab titers, and serum analyte levels on a patient-by-patient basis excluding healthy controls. Patients for which data were unavailable were excluded from correlation analysis for each comparison. Variables on both axes were ordered via hierarchical clustering with the computed dendrogram displayed for subtype frequencies. This dendrogram was divided into 6 groupings with the one containing ISG+ subtypes highlighted in brown. Clinical variables generally correlated with severity highlighted in red and anti-correlated in brown (n for correlation comparisons ranged from n=14-32 individuals). *p-value<0.05, **p-value<0.005, ***p-value<0.0005. FIG. 12B: Scatter plots showing viral load versus levels of antibody binding SARS-COV-2 Nucleocapsid protein for patients in the cohort with severity overlaid. Antibody levels are shown as arbitrary units of MFI from Luminex assay while viral load is represented by an inverse CT number from QRT-PCR with target amplification of the SARS-CoV2 Nucleocapsid sequence. Correlation coefficient and significance calculated using Spearman's method. Patients for which data was unavailable were excluded (M/M, n=9; severe, n=7 patients). FIG. 12C: Matrix of Spearman correlation coefficients between all subtype frequencies (out of major celltypes e.g. Neut ISG represents % out of all Neutrophils) obtained from scRNA-Seq versus protein analyte abundance in plasma as measured using Olink assay on a patient-by-patient basis excluding healthy controls. Patients for which data were unavailable were excluded from correlation analysis for each comparison. Variables on both axes were ordered via hierarchical clustering. ISG subtypes and protein levels strongly correlated with their frequency highlighted in brown. Subtypes and proteins strongly anti-correlated with ISG+subtypes highlighted in red. (n=31 for all comparisons). *p-value<0.05, **p-value<0.005, ***p-value<0.0005. FIG. 12D: Computed total IgG levels in patient sera from ELISA absorbance readings. (n=Apr. 19, 2016 for HC/MM/Severe). FIG. 12E: Longitudinal measurements of anti-Spike and Nucleocapsid antibody levels in patient sera at the indicated days post-enrollment in study. Connected points represent tracking of a single individual. (n=11/8/7/8/6/7/3/7/1/5/0/3 for MM vs. Severe for D0,4,7,14,21,27 respectively). FIG. 12F: Levels of circulating immune complexes (CIC) in patient sera as measured by ELISA with human CIQ used to capture CIC's and an anti-hIgG secondary. Levels shown as heat aggregated human gamma globulin equivalents per mL or (Eq/mL) (n=3/11/9 for HC/MM/Severe respectively). Boxplots represent 25/50/75 percentiles. Statistical testing performed using two-sided Wilcoxon rank-sum test. Boxplot center, median; box limits, 25^{th} and 75^{th} percentile; whiskers, min. and max. data point.

FIG. 13A: Contour plots and histograms of CD14+ monocytes from healthy donor blood cultured with IFNα to induce expression of ISGs and stained with serum from heathy donor, mild/moderate (M/M) or severe SARS-COV-2 positive patients with secondary staining with α-human IgG. FIG. 13B: Geometric MFI of serum staining on CD14+ monocytes treated with IFNα, quantifying data in FIG. 11A. Ctrl, n=4; M/M, n=9; severe, n=7. Boxplot center, median; box limits, 25^{th} and 75^{th} percentile; whiskers, min. and max. data point. FIG. 13C: Summary table of serum staining experiment. Fold change (FC) of α-Human IgG AlexaFluor647 GeoMFI relative to allogeneic healthy donor serum on non-stimulated and IFNα-stimulated healthy PBMCs is listed for each analyzed cell type. Table data cells are color-coded based on degree of FC; gray, FC>2; light gray, 2>FC>1.2; dark gray, FC<1.2. FIG. 13D: Gating strategy for PBMCs to identify different subpopulations. FIG. 13E: Modulation of intermediate to classical CD14 monocytes transition by mild/moderate (orange) and severe (red) patient serum. Each plot represents a single serum sample. Representative experiment from three independent trials and two different healthy PBMC donors. FIG. 13F: Histograms of IFITM3 expression by CD3+CD19+ lymphocytes from healthy donor cultured with IFNα and serum from heathy donor (blue), mild/moderate (orange) and severe (red) SARS-COV-2 positive patients. Mild/Moderate (light yellow) or Severe (pink) sera were pre-treated with protein G/A before incubation with PBMC. Each plot represents a single serum sample. Representative experiment from two independent trials and two different healthy PBMC donors. For FIG. 13A-13F, data from one of two representative experiments is shown. ns, non-significant.

FIG. 14A: Test of ISG neutralization by M/M or severe serum as presented in FIG. 3E, here using sera from a validation cohort of patients. FIG. 14B: Test of ISG neutralization by M/M or severe serum in presence of anti-CD16/CD32/CD64 antibodies to block Fc receptors as presented in FIG. 4A, here using sera from a validation cohort of patients. FIG. 14C: qPCR analysis of IFI27, ISG15 and MXI gene expression in healthy donor PBMCs treated with IFNα with the addition of M/M or severe patient sera with or without Fc receptor blocking (FIG. 4A). Fold changes are relative to untreated healthy donor PBMCs. n=3/group. Data is plotted as mean±SEM. FIG. 14D: Absolute counts of CD14+ monocytes from experiments presented in FIG. 4A (n=16/group). FIG. 14E-14F: Contour plots and histograms of CD14 and IFITM3 expression by monocytes (FIG. 14E) and quantification by Luminex of IL-6, IL-8 in the supernatant (FIG. 14F) from the experiment presented in FIG. 4B and FIG. 4C. FIG. 14G: Boxplots showing fold changes of percentage of IFITM3 positive CD14+ monocytes upon IFNα stimulation normalized to non-treated cells (1 experiment on 2 different pbmc donors: n=8/group). Differences in FIG. 14C and FIG. 14G were calculated using a two-way ANOVA corrected for multiple comparison. *p-value<0.05; **p-value<0.01; ***p-value<0.001; ****p-value<0.0001; ns non-significant. Boxplot center, median; box limits, 25^{th} and 75^{th} percentile; whiskers, min. and max. data point.

### DETAILED DESCRIPTION OF EMBODIMENTS

Before the present methods are described, it is to be understood that the present disclosure is not limited to the particular processes, compositions, or methodologies described, as these may vary. It is also to be understood that the terminology used in the description is for the purposes of describing the particular versions or embodiments only, and is not intended to limit the scope of the present disclosure. Unless defined otherwise, all technical and scientific terms used herein have the same meanings as commonly understood by one of ordinary skill in the art. Although any methods and materials similar or equivalent to those described herein can be used in the practice or testing of embodiments of the present disclosure, the methods, devices, and materials in some embodiments are now described. All publications mentioned herein are incorporated by reference in their entirety. Nothing herein is to be construed as an admission that the present disclosure is not entitled to antedate such disclosure by virtue of prior invention.

### Definitions

Unless otherwise defined herein, scientific and technical terms used in connection with the present disclosure shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of.” “Consisting essentially of,” when used in the claims, shall have its ordinary meaning as used in the field of patent law.

The term “about” is used herein to mean within the typical ranges of tolerances in the art. For example, “about” can be understood as about 2 standard deviations from the mean. According to certain embodiments, when referring to a measurable value such as an amount and the like, “about” is meant to encompass variations of ±20%, ±10%, ±5%, ±1%, ±0.9%, ±0.8%, ±0.7%, ±0.6%, ±0.5%, ±0.4%, ±0.3%, ±0.2% or ±0.1% from the specified value as such variations are appropriate to perform the disclosed methods. When “about” is present before a series of numbers or a range, it is understood that “about” can modify each of the numbers in the series or range.

The term “at least” prior to a number or series of numbers (e.g. “at least two”) is understood to include the number adjacent to the term “at least,” and all subsequent numbers or integers that could logically be included, as clear from context. When “at least” is present before a series of numbers or a range, it is understood that “at least” can modify each of the numbers in the series or range.

Ranges provided herein are understood to include all individual integer values and all subranges within the ranges.

As used herein, the term “animal” includes, but is not limited to, humans and non-human vertebrates such as wild animals, rodents, such as rats, ferrets, and domesticated animals, and farm animals, such as dogs, cats, horses, pigs, cows, sheep, and goats. In some embodiments, the animal is a mammal. In some embodiments, the animal is a human. In some embodiments, the animal is a non-human mammal.

As used herein, the terms “comprising” (and any form of comprising, such as “comprise,” “comprises,” and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

The term “agonist” herein means binds to a receptor and activates the receptor to produce a biological response. In some embodiments, the biological response is depletion of a population of plasma cells or a population of B cells. In some embodiments, the biological response is in a human. In some embodiments, the biological response is increasing a population of neutrophils in a human. In some embodiments, the biological response is increasing the population of cells, such as neutrophils and/or monocyte populations, that secrete interferon.

The term “antagonist” herein binds to a receptor and inactivates the receptor, inhibiting a biological response. In some embodiments, the biological response is the autoinflammatory response or production of antibody production that activates a autoinflammatory response. In some embodiments, the biological response is proliferation or differentiation of a neutrophil population. In some embodiments, the biological response is proliferation or differentiation of a monocyte population. In some embodiments, the biological response is suppression of interferon producing cells, such as monocytes or neutrophils.

The term “antibody,” as used herein, broadly refers to any immunoglobulin (Ig) molecule comprised of four polypeptide chains, two heavy (H) chains and two light (L) chains, or any functional fragment, mutant, variant, or derivative thereof, which retains the essential epitope binding features of an Ig molecule. Such mutant, variant, or derivative antibody formats are known in the art. Non-limiting embodiments of which are discussed below.

In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

As used herein, the term “CDR” refers to the complementarity determining region within antibody variable sequences. In some embodiments, there are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia et al., J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262 (5): 732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc. Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H1}; (ii) V_{H}-C_{H2}; (iii) VH-CH3; (iv) V_{H}-C_{H1}-C_{H2}; (v) V_{H}-C_{H1}-C_{H2}-C_{H3}; (vi) V_{H}-C_{H2}-C_{H3}; (vii) V_{H}-C_{L}; (viii) V_{L}-C_{H1}; (ix) V_{L}-C_{H2}; (x) V_{L}-C_{H3}; (xi) V_{L}-C_{H1}-C_{H2}; (xii) V_{L}-C_{H1}-C_{H2}-C_{H3}; (xiii) V_{L}-C_{H2}-C_{H3}; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric VH or VL domain (e.g., by disulfide bond(s)).

As used herein, the term “fragment” is defined as a physically contiguous portion of the primary structure of a biomolecule. In the case of polypeptides, a fragment may be defined by a contiguous portion of the amino acid sequence of a protein and may be at least about 3-5 amino acids, at least about 6-10 amino acids, at least about 11-15 amino acids, at least about 16-24 amino acids, at least about 25-30 amino acids, at least about 30-45 amino acids and up to the full length of the protein minus a few amino acids. In the case of polynucleotides, a fragment is defined by a contiguous portion of the nucleic acid sequence of a polynucleotide and may be at least about 9-15 nucleotides, at least about 15-30 nucleotides, at least about 31-45 nucleotides, at least about 46-74 nucleotides, at least about 75-90 nucleotides, and at least about 90-130 nucleotides. In some embodiments, fragments of biomolecules are immunogenic fragments.

In some embodiments, the term “functional fragment” means any portion of a polypeptide or amino acid sequence that is of a sufficient length to retain at least partial biological function that is similar to or substantially similar to the wild-type polypeptide or amino acid sequence upon which the fragment is based. If the fragment is a functional fragment of an antibody or antibody-like molecule, the fragment can be immunogenic and therefore possess a binding avidity for one or a plurality of antigens. In some embodiments, a functional fragment of an antibody is a polypeptide that comprises at least about 70%, 75%, 80%, 85%, 90%, 95%, 96%, 97%, 98%, or 99% sequence identity to the full-length polypeptide and has sufficient length to retain at least partial binding affinity to one or a plurality of ligands that bind to the antibody. In some embodiments, a functional fragment has a length of at least about 10, about 20, about 30, about 40, about 50, about 60, about 70, about 80, about 90, or about 100 contiguous amino acids. In some embodiments, the functional fragment is a fragment of the antibodies disclosed herein and has a length of at least about 25, 50, 75, 100, 150, 200, 250, 300, 350, 400, 450, or 500 amino acids.

As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FRI, FR2, FR3 or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

The “variable domain” (variable domain of a light chain (VL), variable domain of a heavy chain (VH)) as used herein denotes each of the pair of light and heavy chains which is involved directly in binding the antibody to the antigen. The domains of variable human light and heavy chains have the same general structure and each domain comprises four framework (FR) regions whose sequences are widely conserved, connected by three “hypervariable regions” (or complementarity determining regions, CDRs). The framework regions adopt a beta-sheet conformation and the CDRs may form loops connecting the beta-sheet structure. The CDRs in each chain are held in their three-dimensional structure by the framework regions and form together with the CDRs from the other chain an antigen binding site. References to “VH” refer to the variable domain of an immunoglobulin heavy chain, including that of an antibody fragment, such as Fv, scFv, dsFv or Fab. References to “VL” refer to the variable domain of an immunoglobulin light chain, including that of an Fv, scFv, dsFv or Fab.

The term “antigen binding portion” or “antigen binding fragment” of an antibody (or simply “antibody portion” or “antibody fragment”), as used herein, refers to one or more fragments of an antibody that retain the ability to specifically bind to an antigen (e.g., hCD40). It has been shown that the antigen-binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen-binding portion” or “antigen binding fragment” of an antibody include (i) a Fab fragment, a monovalent fragment consisting of the VL, VH, CL and CHI domains; (ii) a F(ab′) 2 fragment, a bivalent fragment comprising two Fab fragments linked by a disulfide bridge at the hinge region; (iii) a Fd fragment consisting of the VH and CHI domains; (iv) a Fv fragment consisting of the VL and VH domains of a single arm of an antibody, (v) a dAb fragment (Ward et al., (1989) Nature 341:544-546, Winter et al., PCT publication WO 90/05144 A1 herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” or “antigen binding fragment” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such antibody binding portions are known in the art (Kontermann and Dubel eds., Antibody Engineering (2001) Springer-Verlag. New York. 790 pp. (ISBN 3-540-41354-5).

Full length antibodies comprise immunoglobulin constant regions of one or more immunoglobulin classes. Immunoglobulin classes include IgG, IgM, IgA, IgD, and IgE isotypes and, in the case of IgG and IgA, their subtypes. In a preferred embodiment, a full length antibody of the disclosure has a constant domain structure of an IgG type antibody.

The terms “Kabat numbering,” “Kabat definitions” and “Kabat labeling” are used interchangeably herein. These terms, which are recognized in the art, refer to a system of numbering amino acid residues which are more variable (i.e., hypervariable) than other amino acid residues in the heavy and light chain variable regions of an antibody, or an antigen-binding portion thereof (Kabat et al. (1971) Ann. NY Acad, Sci. 190:382-391; Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 44 to 51 for CDR1, amino acid positions 69 to 75 for CDR2, and amino acid positions 112 to 120 for CDR3. For the light chain variable region, the hypervariable region ranges from amino acid positions 49 to 55 for CDR1, amino acid positions 73 to 75 for CDR2, and amino acid positions 112 to 121 for CDR3.

The term “multispecific antibody” refers to an antibody or antibody-like molecule, or fragment thereof, capable of binding two or more related or unrelated targets, or antigens. Antibody specificity refers to selective recognition of the antibody for a particular epitope, or amino acid sequence, of an antigen. Natural antibodies, for example, are monospecific. Bispecific antibodies are antibodies which have two different antigen-binding specificities. Trispecific antibodies accordingly are antibodies of the disclosure which have three different antigen-binding specificities. Tetraspecific antibodies according to the disclosure are antibodies which have four different antigen-binding specificities.

The term “immunogenic fragment” or “epitope” includes any polypeptide determinant capable of specific binding to an antibody. In certain embodiments, immunogenic fragment or epitope determinant include chemically active surface groupings of molecules such as amino acids, sugar side chains, phosphoryl, or sulfonyl, and, in certain embodiments, may have specific three dimensional structural characteristics, and or specific charge characteristics. An immunogenic fragment or epitope is a region of an antigen that is bound by an antibody.

The term “antigen” refers to a polypeptide that can stimulate the production of antibodies or a T cell response in an animal, including polypeptides that are injected or absorbed into an animal. An antigen reacts with the products of specific humoral or cellular immunity.

The term “human antibody,” as used herein, is intended to include non-naturally occurring human antibodies. The term includes antibodies that are recombinantly produced in a non-human mammal, or in cells of a non-human mammal. The term is not intended to include antibodies isolated from or generated in a human subject. Human antibodies can be produced using various techniques known in the art, including phage display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991); van Dijk and van de Winkel, Curr. Opin. Pharmacol., 5:368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B cell hybridoma technology.

The antibodies of the disclosure are, in some embodiments, recombinant antibodies. The term “recombinant antibody,” as used herein, is intended to include all antibodies that are prepared, expressed, created or isolated by recombinant means, such as antibodies expressed using a recombinant expression vector transfected into a host cell (described further below), antibodies isolated from a recombinant, combinatorial antibody library (described further below), antibodies isolated from an animal (e.g., a mouse) that is transgenic for immunoglobulin (e.g. human) genes (see e.g., Taylor et al. (1992) Nucl. Acids Res. 20:6287-6295) or antibodies prepared, expressed, created or isolated by any other means that involves splicing of one species of immunoglobulin gene sequences to other DNA sequences. In certain embodiments, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while related to human germline VH and VL sequences, may not naturally exist within the human antibody germline repertoire in vivo. Recombinant antibodies can be from any mammal, such as, but not limited to, human, rat, mouse, rabbit, dog, horse, pig, etc.

The antibodies of the disclosure can be isolated antibodies. An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present invention. An isolated antibody also includes an antibody in situ within a recombinant cell. Isolated antibodies are antibodies that have been subjected to at least one purification or isolation step. According to certain embodiments, an isolated antibody can be substantially free of other cellular material and/or chemicals.

In some embodiments, the antibody of the disclosure is a humanized antibodies. A “humanized” antibody (or antigen-binding fragment thereof), as used herein, is an antibody (or antigen-binding fragment thereof) that retains the reactivity of a non-human antibody while being less immunogenic in humans. This can be achieved, for instance, by retaining the non-human CDR regions and replacing the remaining parts of the antibody with their human counterparts (i.e., the constant region as well as the framework portions of the variable region). See, e.g., Morrison et al., Proc. Natl. Acad. Sci. USA, 81:6851-6855, 1984; Morrison and Oi, Adv. Immunol., 44:65-92, 1988; Verhoeyen et al., Science, 239:1534-1536, 1988; Padlan, Molec. Immun, 28:489-498, 1991; and Padlan, Molec. Immun, 31:169-217, 1994. Other examples of human engineering technology include, but is not limited to Xoma technology disclosed in U.S. Pat. No. 5,766,886.

The term “kₒₙ,” as used herein, is intended to refer to the on rate constant for association of an antibody to the antigen to form the antibody/antigen complex as is known in the art.

The term “k_{off},” as used herein, is intended to refer to the off rate constant for dissociation of an antibody from the antibody/antigen complex as is known in the art.

The term “K_{D},” as used herein, is intended to refer to the dissociation constant of a particular antibody-antigen interaction as is known in the art.

The terms “specific binding,” “specifically binds” or “specifically binding,” as used herein in the context of an antibody, including a multispecific antibody, refer to non-covalent or covalent preferential binding of an antibody to an antigen relative to other molecules or moieties (e.g., an antibody specifically binds to a particular antigen relative to other available antigens). In some embodiments, an antibody specifically binds to an antigen (e.g., an epitope in the CD32-binding site) if it binds to the antigen with a dissociation constant K_{D} of 1×10⁻⁹ M or less (e.g., 1×10⁻¹⁰ M or less, 1×10⁻¹¹ M or less, 10⁻¹² M or less).

A “blocking” antibody or an “antagonist” antibody is one that inhibits or reduces a biological activity of the antigen it binds. In some embodiments, blocking antibodies or antagonist antibodies substantially or completely inhibit the biological activity of the antigen.

An “agonist” or activating antibody is one that enhances or initiates signaling by the antigen to which it binds. In some embodiments, agonist antibodies cause or activate signaling without the presence of the natural ligand.

As used herein, the terms “CD32” (cluster of differentiation 32), “Fc-gamma receptor II,” “FcγRII” and “FCGR2” are used interchangeably and refer to a 40 kDa surface receptor glycoprotein belonging to the Ig gene superfamily. CD32 is a low affinity receptor for IgG complexes and is expressed on a wide variety of cell types, including B lymphocytes, eosinophils, monocytes, granulocytes and platelets. In humans, there are three major CD32 subtypes: CD32a, CD32b, and CD32c. Subtypes CD32a and CD32c are involved in activating cellular responses, while subtype CD32b is inhibitory.

An “anti-CD32b antibody” is an antibody that binds specifically to the CD32b antigen, or an immunogenic fragment or epitope thereof.

As used herein, the phrase “in need thereof” means that the animal or mammal has been identified or suspected as having a need for the particular method or treatment. In some embodiments, the identification can be by any means of diagnosis or observation. In any of the methods and treatments described herein, the animal or mammal can be in need thereof.

As used herein, the term “mammal” means any animal in the class Mammalia such as rodent (i.e., mouse, rat, or guinea pig), monkey, cat, dog, cow, horse, pig, or human. In some embodiments, the mammal is a human. In some embodiments, the mammal refers to any non-human mammal. The present disclosure relates to any of the methods or compositions of matter wherein the sample is taken from a mammal or non-human mammal. The present disclosure relates to any of the methods or compositions of matter wherein the sample is taken from a human or non-human primate.

As used herein, the term “subject,” “individual” or “patient,” used interchangeably, means any animal, including mammals, such as mice, rats, other rodents, rabbits, dogs, cats, swine, cattle, sheep, horses, or primates, such as humans. In some embodiments, the subject is a human having a disease or disorder. In some embodiments, the subject is a healthy human being. In some embodiments, the subject is a human suspected of having or being identified as at risk to develop Coronaviridae infection. In some embodiments, the subject is a human suspected of having or being identified as at risk to develop SARS-COV-2 infection. In some embodiments, the subject is a human suspected of having or has been diagnosed with Coronaviridae infection. In some embodiments, the subject is a human suspected of having or has been diagnosed with SARS-COV-2 infection. In some embodiments, the subject is a human suspected of having or being identified as at risk to develop severe response against Coronaviridae infection. In some embodiments, the subject is a human suspected of having or being identified as at risk to develop severe response against SARS-COV-2 infection. In some embodiments, the subject is a human being treated or assessed for Coronaviridae infection. In some embodiments, the subject is a human being treated or assessed for SARS-COV-2 infection.

The “percent identity” or “percent homology” of two polynucleotide or two polypeptide sequences is determined by comparing the sequences using the GAP computer program (a part of the GCG Wisconsin Package, version 10.3 (Accelrys, San Diego, Calif.)) using its default parameters. “Identical” or “identity” as used herein in the context of two or more nucleic acids or amino acid sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0. Briefly, the BLAST algorithm, which stands for Basic Local Alignment Search Tool is suitable for determining sequence similarity. Software for performing BLAST analyses is publicly available through the National Center for Biotechnology Information (ncbi.nlm.nih.gov). This algorithm involves first identifying high scoring sequence pair (HSPs) by identifying short words of length Win the query sequence that either match or satisfy some positive-valued threshold score T when aligned with a word of the same length in a database sequence. T is referred to as the neighborhood word score threshold (Altschul et al., supra). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P (N)), which provides an indication of the probability by which a match between two nucleotide sequences would occur by chance. For example, a nucleic acid is considered similar to another if the smallest sum probability in comparison of the test nucleic acid to the other nucleic acid is less than about 1, less than about 0.1, less than about 0.01, and less than about 0.001.

As used herein, the terms “treat,” “treated,” or “treating” can refer to therapeutic treatment and/or prophylactic or preventative measures wherein the object is to prevent or slow down (lessen) an undesired physiological condition, disorder or disease, or obtain beneficial or desired clinical results. For purposes of the embodiments described herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment can also include eliciting a clinically significant response without excessive levels of side effects. Treatment also includes prolonging survival as compared to expected survival if not receiving treatment.

As used herein, the term “therapeutic” means an agent utilized to treat, combat, ameliorate, prevent or improve an unwanted condition or disease of a patient.

A “therapeutically effective amount” or “effective amount” of a composition is a predetermined amount calculated to achieve the desired effect, i.e., to treat, combat, ameliorate, prevent or improve one or more symptoms of a viral infection. The activity contemplated by the present methods includes both medical therapeutic and/or prophylactic treatment, as appropriate. The specific dose of a compound administered according to the present disclosure to obtain therapeutic and/or prophylactic effects will, of course, be determined by the particular circumstances surrounding the case, including, for example, the compound administered, the route of administration, and the condition being treated. It will be understood that the effective amount administered will be determined by the physician in the light of the relevant circumstances including the condition to be treated, the choice of compound to be administered, and the chosen route of administration, and therefore the above dosage ranges are not intended to limit the scope of the present disclosure in any way. A therapeutically effective amount of compounds of embodiments of the present disclosure is typically an amount such that when it is administered in a physiologically tolerable excipient composition, it is sufficient to achieve an effective systemic concentration or local concentration in the tissue.

A “pharmaceutically acceptable carrier” refers to an ingredient in a pharmaceutical formulation, other than an active ingredient, which is nontoxic to a subject. Pharmaceutically acceptable carriers includes any and all solvents, dispersion media, coatings, antibacterial and antifungal agents, isotonic and absorption delaying agents, and the like that are physiologically compatible. In some embodiments, the carrier is suitable for intravenous, intramuscular, subcutaneous, parenteral, spinal or epidermal administration (e.g. by injection or infusion).

The pharmaceutical compositions according to the disclosure may also contain adjuvants such as preservatives, wetting agents, emulsifying agents and dispersing agents. Prevention of presence of microorganisms may be ensured both by sterilization procedures and by the inclusion of various antibacterial and antifungal agents, for example, paraben, chlorobutanol, phenol, sorbic acid, and the like. In some embodiments, the pharmaceutical composition comprises one or a plurality of isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

### Anti-CD32 Antibodies

CD32b is an integral membrane glycoprotein and is the predominant Fc receptor (FcR) (Amigorena et al. (1992) Science 256:1808-1812; Takai (2002) Nat. Rev. Immunol 2:580-592). The CD32b gene is expressed on B lymphocytes and its extracellular domain is 96% identical to CD32a (also known as FcγRIIA). CD32a is highly expressed by myeloid cells and is absent in B cells (Takai (2002) Nat. Rev. Immunol 2:580-592; Ravetch et al. (2001) Annu. Rev. Immunol. 19:275-290), and they bind IgG complexes in an indistinguishable manner but create two functionally heterogeneous responses to receptor ligation. There are 5 isoforms of CD32b known to date with the following sequences.

>sp|P31994|FCG2B_HUMAN Low affinity immunoglobulin
gamma Fc region receptor
II-b (Homo sapiens)
(SEQ ID NO: 757)
MGILSFLPVLATESDWADCKSPQPWGHMLLWTAVLFLAPVAGTPAAPPKA
VLKLEPQWINVLQEDSVTLTCRGTHSPESDSIQWFHNGNLIPTHTQPSYR
FKANNNDSGEYTCQTGQTSLSDPVHLTVLSEWLVLQTPHLEFQEGETIVL
RCHSWKDKPLVKVTFFQNGKSKKFSRSDPNFSIPQANHSHSGDYHCTGNI
GYTLYSSKPVTITVQAPSSSPMGIIVAVVTGIAVAAIVAAVVALIYCRKK
RISALPGYPECREMGETLPEKPANPTNPDEADKVGAENTITYSLLMHPDA
LEEPDDQNRI
>sp|P31994-2|FCG2B_HUMAN Isoform IIB2 of Low
affinity immunoglobulin gamma Fc
region receptor II-b (Homo sapiens)
(SEQ ID NO: 758)
MGILSFLPVLATESDWADCKSPQPWGHMLLWTAVLFLAPVAGTPAAPPKA
VLKLEPQWINVLQEDSVTLTCRGTHSPESDSIQWFHNGNLIPTHTQPSYR
FKANNNDSGEYTCQTGQTSLSDPVHLTVLSEWLVLQTPHLEFQEGETIVL
RCHSWKDKPLVKVTFFQNGKSKKFSRSDPNFSIPQANHSHSGDYHCTGNI
GYTLYSSKPVTITVQAPSSSPMGIIVAVVTGIAVAAIVAAVVALIYCRKK
RISANPTNPDEADKVGAENTITYSLLMHPDALEEPDDQNRI
>sp|P31994-3|FCG2B_HUMAN Isoform IIB3 of Low
affinity immunoglobulin gamma Fc
region receptor II-b (Homo sapiens)
(SEQ ID NO: 759)
MGILSFLPVLATESDWADCKSPQPWGHMLLWTAVLFLAAPPKAVLKLEPQ
WINVLQEDSVTLTCRGTHSPESDSIQWFHNGNLIPTHTQPSYRFKANNND
SGEYTCQTGQTSLSDPVHLTVLSEWLVLQTPHLEFQEGETIVLRCHSWKD
KPLVKVTFFQNGKSKKFSRSDPNFSIPQANHSHSGDYHCTGNIGYTLYSS
KPVTITVQAPSSSPMGIIVAVVTGIAVAAIVAAVVALIYCRKKRISALPG
YPECREMGETLPEKPANPTNPDEADKVGAENTITYSLLMHPDALEEPDDQ
NRI
>sp|P31994-4|FCG2B_HUMAN Isoform 4 of Low
affinity immunoglobulin gamma Fc
region receptor II-b (Homo sapiens)
(SEQ ID NO: 760)
MGILSFLPVLATESDWADCKSPQPWGHMLLWTAVLFLAPVAGTPAPPKAV
LKLEPQWINVLQEDSVTLTCRGTHSPESDSIQWFHNGNLIPTHTQPSYRF
KANNNDSGEYTCQTGQTSLSDPVHLTVLSEWLVLQTPHLEFQEGETIVLR
CHSWKDKPLVKVTFFQNGKSKKFSRSDPNFSIPQANHSHSGDYHCTGNIG
YTLYSSKPVTITVQAPSSSPMGIIVAVVTGIAVAAIVAAVVALIYCRKKR
ISALPGYPECREMGETLPEKPANPTNPDEADKVGAENTITYSLLMHPDAL
EEPDDQNRI
>sp|P31994-5|FCG2B_HUMAN Isoform 5 of Low
affinity immunoglobulin gamma Fc
region receptor II-b (Homo sapiens)
(SEQ ID NO: 761)
MGILSFLPVLATESDWADCKSPQPWGHMLLWTAVLFLAPVAGTPAPPKAV
LKLEPQWINVLQEDSVTLTCRGTHSPESDSIQWFHNGNLIPTHTQPSYRF
KANNNDSGEYTCQTGQTSLSDPVHLTVLSEWLVLQTPHLEFQEGETIVLR
CHSWKDKPLVKVTFFQNGKSKKFSRSDPNFSIPQANHSHSGDYHCTGNIG
YTLYSSKPVTITVQAPSSSPMGIIVAVVTGIAVAAIVAAVVALIYCRKKR
ISANPTNPDEADKVGAENTITYSLLMHPDALEEPDDQNRI

The terms “CD32b” and “CD32b antigen” are used interchangeably herein and, unless specified otherwise, include any variants, isoforms and species homologs of human CD32b which are naturally expressed by cells or are expressed on cells transfected with the CD32b gene. However, these terms do not include CD32a or CD32c. In some embodiments, the term “CD32b” refers to a polypeptide comprising at least about 70%, 75%, 80%, 85%, 90%, 95%, 96%, 97%, 98% or 99% sequence identity to any of SEQ ID NO: 757, SEQ ID NO: 758, SEQ ID NO: 759, SEQ ID NO: 760 or SEQ ID NO: 761. In some embodiments, the term “CD32b” refers to a polypeptide comprising the amino acid sequence of SEQ ID NO: 757, SEQ ID NO: 758, SEQ ID NO: 759, SEQ ID NO: 760 or SEQ ID NO: 761.

In some embodiments, anti-CD32 antibodies, or antigen-binding fragment, variant, or derivative thereof, useful for the methods of the disclosure are specific for CD32b comprising at least about 70%, 75%, 80%, 85%, 90%, 95%, 96%, 97%, 98%, 99% or 100% sequence identity to SEQ ID NO: 757, or immunogenic fragments or epitopes thereof. In some embodiments, anti-CD32 antibodies, or antigen-binding fragment, variant, or derivative thereof, useful for the methods of the disclosure are specific for CD32b comprising at least about 70%, 75%, 80%, 85%, 90%, 95%, 96%, 97%, 98%, 99% or 100% sequence identity to SEQ ID NO: 758, or immunogenic fragments or epitopes thereof. In some embodiments, anti-CD32 antibodies, or antigen-binding fragment, variant, or derivative thereof, useful for the methods of the disclosure are specific for CD32b comprising at least about 70%, 75%, 80%, 85%, 90%, 95%, 96%, 97%, 98%, 99% or 100% sequence identity to SEQ ID NO: 759, or immunogenic fragments or epitopes thereof. In some embodiments, anti-CD32 antibodies, or antigen-binding fragment, variant, or derivative thereof, useful for the methods of the disclosure are specific for CD32b comprising at least about 70%, 75%, 80%, 85%, 90%, 95%, 96%, 97%, 98%, 99% or 100% sequence identity to SEQ ID NO: 760, or immunogenic fragments or epitopes thereof. In some embodiments, anti-CD32 antibodies, or antigen-binding fragment, variant, or derivative thereof, useful for the methods of the disclosure are specific for CD32b comprising at least about 70%, 75%, 80%, 85%, 90%, 95%, 96%, 97%, 98%, 99% or 100% sequence identity to SEQ ID NO: 761, or immunogenic fragments or epitopes

In some embodiments, anti-CD32 antibodies, or antigen-binding fragment, variant, or derivative thereof, binds to human CD32b with a K_{D} of about 5×10⁻⁸ M or less, binds to human CD32b with a K_{D} of about 4×10⁻⁸ M or less, binds to human CD32b with a K_{D} of about 3×10⁻⁸ M or less, binds to human CD32b with a K_{D} of about 2×10⁻⁸ M or less, binds to human CD32b with a K_{D} of about 1×10⁻⁸ M or less, or binds to human CD32b with a K_{D} of about 9×10⁻⁹ M or less. In some embodiments, anti-CD32 antibodies, or antigen-binding fragment, variant, or derivative thereof, bind to human CD32b with a K_{D} from about 1 micromolar to about 1 nanomolar. In some embodiments, anti-CD32 antibodies, or antigen-binding fragment, variant, or derivative thereof, bind to human CD32b with a K_{D} from about 1 micromolar to about 1 nanomolar. In some embodiments, anti-CD32 antibodies, or antigen-binding fragment, variant, or derivative thereof, bind to human CD32b with a K_{D} from about 1 micromolar to about 100 nanomolar. In some embodiments, anti-CD32 antibodies, or antigen-binding fragment, variant, or derivative thereof, bind to human CD32b with a K_{D} from about 1 micromolar to about 200 nanomolar. In some embodiments, anti-CD32 antibodies, or antigen-binding fragment, variant, or derivative thereof, bind to human CD32b with a K_{D} from about 1 micromolar to about 300 nanomolar. In some embodiments, anti-CD32 antibodies, or antigen-binding fragment, variant, or derivative thereof, bind to human CD32b with K_{D} from about 1 micromolar to about 400 nanomolar. In some embodiments, anti-CD32 antibodies, or antigen-binding fragment, variant, or derivative thereof, bind to human CD32b with a K_{D} from about 1 micromolar to about 500 nanomolar. In some embodiments, anti-CD32 antibodies, or antigen-binding fragment, variant, or derivative thereof, bind to human CD32b with a K_{D} from about 500 nanomolar to about 1 nanomolar. In some embodiments, anti-CD32 antibodies, or antigen-binding fragment, variant, or derivative thereof, bind to human CD32b with a K_{D} from about 400 nanomolar to about 1 nanomolar. In some embodiments, anti-CD32 antibodies, or antigen-binding fragment, variant, or derivative thereof, bind to human CD32b with a K_{D} from about 300 nanomolar to about 1 nanomolar. In some embodiments, anti-CD32 antibodies, or antigen-binding fragment, variant, or derivative thereof, bind to human CD32b with a K_{D} from about 200 nanomolar to about 1 nanomolar. In some embodiments, anti-CD32 antibodies, or antigen-binding fragment, variant, or derivative thereof, bind to human CD32b with a K_{D} from about 100 nanomolar to about 1 nanomolar. In some embodiments, anti-CD32 antibodies, or antigen-binding fragment, variant, or derivative thereof, bind to human CD32b with a K_{D} from about 400 nanomolar to about 500 picomolar. In some embodiments, anti-CD32 antibodies, or antigen-binding fragment, variant, or derivative thereof, bind to human CD32b with a K_{D} from about from about 200 nanomolar to about 500 picomolar. In some embodiments, anti-CD32 antibodies, or antigen-binding fragment, variant, or derivative thereof, bind to human CD32b with a K_{D} from about from about 100 nanomolar to about 800 picomolar. In some embodiments, anti-CD32 antibodies, or antigen-binding fragment, variant, or derivative thereof, bind to human CD32b with a K_{D} from about from about 300 nanomolar to about 800 picomolar but does not bind to human CD32a. In some embodiments, anti-CD32 antibodies, or antigen-binding fragment, variant, or derivative thereof, bind human CD32b with any of the aforementioned K_{D} values and bind to human CD32a with a K_{D} of greater than about 400 nanomolar, 500 nanomolar, 600 nanomolar, 700 nanomolar, 800 nanomolar, 900 nanomolar or 1 micromolar.

The antibody can be a full-length antibody or an antibody fragment that retains its binding ability and the antibody can be of any isotype. In some embodiments, the antibody is a full-length antibody of an IgG1 isotype. In some embodiments, the antibody is an antibody fragment, such as a Fab fragment. In some embodiments, the antibody is a single chain antibody, such as scFv.

Standard assays to evaluate the binding ability of the antibodies toward CD32b are known in the art, including for example, ELISA and flow cytometry. The binding kinetics (e.g., binding affinity) of the antibodies also can be assessed by standard assays known in the art, such as by Biacore analysis. Other assays for evaluating the properties described above may include, but not limited to, flow cytometric analyses to evaluate down-modulation of surface expression of CD32b and EA-rosetting assays to evaluate inhibition of CD32b ligand binding.

Suitable anti-CD32 antibodies, or antigen-binding fragment, variant, or derivative thereof, useful for the methods of the disclosure may include those provided in Table 1 and described in, for example, U.S. Pat. No. 9,663,578 and U.S. Patent Application Publication No. 2017/0198040, as well as Lu et al., “Development of Anti-CD32b Antibodies with Enhanced Fc Function for the Treatment of B and Plasma Cell Malignancies,” Mol. Cancer Ther., 2020, 19 (10): 2089-2104, all of which are expressly incorporated herein by reference.

TABLE 1
Anti-CD32 antibodies, or antigen-binding fragment, variant, or derivative thereof.

| | | |
|---|---|---|
| SEQ ID NO | Description | Sequence |

Antibody 001

| | | |
|---|---|---|
| 1 | HCDR1 | GGTFSDYAIS |
| | (Combined) | |
| 2 | HCDR2 | GIIPISGTANYAQKFQG |
| | (Combined) | |
| 3 | HCDR3 | DHSSSSYDYQYGLAV |
| | (Combined) | |
| 4 | HCDR1 | DYAIS |
| | (Kabat) | |
| 5 | HCDR2 | GIIPISGTANYAQKFQG |
| | (Kabat) | |
| 6 | HCDR3 | DHSSSSYDYQYGLAV |
| | (Kabat) | |
| 7 | HCDR1 | GGTFSDY |
| | (Chothia) | |
| 8 | HCDR2 | IPISGT |
| | (Chothia) | |
| 9 | HCDR3 | DHSSSSYDYQYGLAV |
| | (Chothia) | |
| 10 | VH | QVQLVQSGAEVKKPGSSVKVSCKASGGTFSDYAISVWRQAPGQ |
| | | GLEWMGGIIPISGTANYAQKFQGRVTITADESTSTAYMELSSLRS |
| | | EDTAVYYCARDHSSSSYDYQYGLAVWGQGTLVTVSS |
| 11 | VH DNA | CAGGTGCAGCTGGTGCAGTCAGGCGCCGAAGTGAAGAAACC |
| | | CGGCTCTAGCGTGAAAGTCAGCTGTAAAGCTAGTGGCGGCAC |
| | | CTTTAGCGACTACGCTATTAGCTGGGTCAGACAGGCCCCAGG |
| | | TCAGGGCCTGGAGTGGATGGGCGGAATTATCCCTATTAGCGG |
| | | CACCGCTAACTACGCTCAGAAATTTCAGGGTAGAGTGACTATC |
| | | ACCGCCGACGAGTCTACTAGCACCGCCTATATGGAACTGTCT |
| | | AGCCTGAGATCAGAGGACACCGCCGTCTACTACTGCGCTAGA |
| | | GATCACTCTAGCTCTAGCTACGACTATCAGTACGGCCTGGCC |
| | | GTGTGGGGTCAGGGCACCCTGGTCACCGTGTCTAGC |
| 12 | Heavy Chain | QVQLVQSGAEVKKPGSSVKVSCKASGGTFSDYAISVWRQAPGQ |
| | | GLEWMGGIIPISGTANYAQKFQGRVTITADESTSTAYMELSSLRS |
| | | EDTAVYYCARDHSSSSYDYQYGLAVWGQGTLVTVSSASTKGPS |
| | | VFPLAPSSKSTSGGTAALGCLVKDYFPEPVTVSWNSGALTSGVH |
| | | TFPAVLQSSGLYSLSSVVTVPSSSLGTQTYICNVNHKPSNTKVDK |
| | | RVEPKSCDKTHTCPPCPAPELLGGPSVFLFPPKPKDTLMISRTPE |
| | | VTCVVVDVSHEDPEVKFNWYVDGVEVHNAKTKPREEQYNSTYR |
| | | VVSVLTVLHQDWLNGKEYKCKVSNKALPAPIEKTISKAKGQPREP |
| | | QVYTLPPSREEMTKNQVSLTCLVKGFYPSDIAVEWESNGQPENN |
| | | YKTTPPVLDSDGSFFLYSKLTVDKSRWGQGNVFSCSVMHEALHN |
| | | HYTQKSLSLSPGK |
| 13 | Heavy Chain | CAGGTGCAGCTGGTGCAGTCAGGCGCCGAAGTGAAGAAACC |
| | DNA | CGGCTCTAGCGTGAAAGTCAGCTGTAAAGCTAGTGGCGGCAC |
| | | CTTTAGCGACTACGCTATTAGCTGGGTCAGACAGGCCCCAGG |
| | | TCAGGGCCTGGAGTGGATGGGCGGAATTATCCCTATTAGCGG |
| | | CACCGCTAACTACGCTCAGAAATTTCAGGGTAGAGTGACTATC |
| | | ACCGCCGACGAGTCTACTAGCACCGCCTATATGGAACTGTCT |
| | | AGCCTGAGATCAGAGGACACCGCCGTCTACTACTGCGCTAGA |
| | | GATCACTCTAGCTCTAGCTACGACTATCAGTACGGCCTGGCC |
| | | GTGTGGGGTCAGGGCACCCTGGTCACCGTGTCTAGCGCTAG |
| | | CACTAAGGGCCCAAGTGTGTTTCCCCTGGCCCCCAGCAGCAA |
| | | GTCTACTTCCGGCGGAACTGCTGCCCTGGGTTGCCTGGTGAA |
| | | GGACTACTTCCCCGAGCCCGTGACAGTGTCCTGGAACTCTGG |
| | | GGCTCTGACTTCCGGCGTGCACACCTTCCCCGCCGTGCTGCA |
| | | GAGCAGCGGCCTGTACAGCCTGAGCAGCGTGGTGACAGTGC |
| | | CCTCCAGCTCTCTGGGAACCCAGACCTATATCTGCAACGTGA |
| | | ACCACAAGCCCAGCAACACCAAGGTGGACAAGAGAGTGGAG |
| | | CCCAAGAGCTGCGACAAGACCCACACCTGCCCCCCCTGCCC |
| | | AGCTCCAGAACTGCTGGGAGGGCCTTCCGTGTTCCTGTTCCC |
| | | CCCCAAGCCCAAGGACACCCTGATGATCAGCAGGACCCCCG |
| | | AGGTGACCTGCGTGGTGGTGGACGTGTCCCACGAGGACCCA |
| | | GAGGTGAAGTTCAACTGGTACGTGGACGGCGTGGAGGTGCA |
| | | CAACGCCAAGACCAAGCCCAGAGAGGAGCAGTACAACAGCA |
| | | CCTACAGGGTGGTGTCCGTGCTGACCGTGCTGCACCAGGACT |
| | | GGCTGAACGGCAAAGAATACAAGTGCAAAGTCTCCAACAAGG |
| | | CCCTGCCAGCCCCAATCGAAAAGACAATCAGCAAGGCCAAGG |
| | | GCCAGCCACGGGAGCCCCAGGTGTACACCCTGCCCCCCAGC |
| | | CGGGAGGAGATGACCAAGAACCAGGTGTCCCTGACCTGTCTG |
| | | GTGAAGGGCTTCTACCCCAGCGATATCGCCGTGGAGTGGGA |
| | | GAGCAACGGCCAGCCCGAGAACAACTACAAGACCACCCCCC |
| | | CAGTGCTGGACAGCGACGGCAGCTTCTTCCTGTACAGCAAGC |
| | | TGACCGTGGACAAGTCCAGGTGGCAGCAGGGCAACGTGTTC |
| | | AGCTGCAGCGTGATGCACGAGGCCCTGCACAACCACTACACC |
| | | CAGAAGTCCCTGAGCCTGAGCCCCGGCAAG |
| 14 | LCDR1 | SGDKLGDYYVH |
| | (Combined) | |
| 15 | LCDR2 | QDSKRPS |
| | (Combined) | |
| 16 | LCDR3 | GATDLSPWSIV |
| | (Combined) | |
| 17 | LCDR1 | SGDKLGDYYVH |
| | (Kabat) | |
| 18 | LCDR2 | QDSKRPS |
| | (Kabat) | |
| 19 | LCDR3 | GATDLSPWSIV |
| | (Kabat) | |
| 20 | LCDR1 | DKLGDYY |
| | (Chothia) | |
| 21 | LCDR2 | QDS |
| | (Chothia) | |
| 22 | LCDR3 | TDLSPWSI |
| | (Chothia) | |
| 23 | VL | DIELTQPPSVSVSPGETASITCSGDKLGDYYVHWYQQKPGGAPV |
| | | LVIYQDSKRPSGIPERFSGSNSGNTATLTISGTQAEDEADYYCGAT |
| | | DLSPWSIVFGGGTKLTVL |
| 24 | VH DNA | GATATCGAGCTGACTCAGCCCCCTAGCGTCAGCGTCAGCCCT |
| | | GGCGAGACAGCCTCTATCACCTGTAGCGGCGATAAGCTGGG |
| | | CGACTACTACGTGCACTGGTATCAGCAGAAGCCCGGTCAGGC |
| | | CCCCGTGCTGGTGATCTATCAGGACTCTAAGCGGCCTAGCGG |
| | | AATCCCCGAGCGGTTTAGCGGCTCTAATAGCGGTAACACCGC |
| | | TACCCTGACTATTAGCGGCACTCAGGCCGAGGACGAGGCCG |
| | | ACTACTACTGCGGCGCTACCGACCTGAGCCCCTGGTCTATCG |
| | | TGTTCGGCGGAGGCACTAAGCTGACCGTGCTG |
| 25 | Light Chain | DIELTQPPSVSVSPGETASITCSGDKLGDYYVHWYQQKPGQAPV |
| | | LVIYQDSKRPSGIPERFSGSNSGNTATLTISGTQAEDEADYYCGA |
| | | TDLSPWSIVFGGGTKLTVLGQPKAAPSVTLFPPSSEELQANKATL |
| | | VCLISDFYPGAVTVAWKADSSPVKAGVETTTPSKQSNNKYAASS |
| | | YLSLTPEQWKSHRSYSCQVTHEGSTVEKTVAPTECS |
| 26 | Light Chain | GATATCGAGCTGACTCAGCCCCCTAGCGTCAGCGTCAGCCCT |
| | DNA | GGCGAGACAGCCTCTATCACCTGTAGCGGCGATAAGCTGGG |
| | | CGACTACTACGTGCACTGGTATCAGCAGAAGCCCGGTCAGGC |
| | | CCCCGTGCTGGTGATCTATCAGGACTCTAAGCGGCCTAGCGG |
| | | AATCCCCGAGCGGTTTAGCGGCTCTAATAGCGGTAACACCGC |
| | | TACCCTGACTATTAGCGGCACTCAGGCCGAGGACGAGGCCG |
| | | ACTACTACTGCGGCGCTACCGACCTGAGCCCCTGGTCTATCG |
| | | TGTTCGGCGGAGGCACTAAGCTGACCGTGCTGGGTCAGCCTA |
| | | AGGCTGCCCCCAGCGTGACCCTGTTCCCCCCCAGCAGCGAG |
| | | GAGCTGCAGGCCAACAAGGCCACCCTGGTGTGCCTGATCAG |
| | | CGACTTCTACCCAGGCGCCGTGACCGTGGCCTGGAAGGCCG |
| | | ACAGCAGCCCCGTGAAGGCCGGCGTGGAGACCACCACCCCC |
| | | AGCAAGCAGAGCAACAACAAGTACGCCGCCAGCAGCTACCTG |
| | | AGCCTGACCCCCGAGCAGTGGAAGAGCCACAGGTCCTACAG |
| | | CTGCCAGGTGACCCACGAGGGCAGCACCGTGGAAAAGACCGT |
| | | GGCCCCAACCGAGTGCAGC |

Antibody 002

| | | |
|---|---|---|
| 27 | HCDR1 | GGTFSDYAIS |
| | (Combined) | |
| 28 | HCDR2 | GIIPISGTANYAQKFQG |
| | (Combined) | |
| 29 | HCDR3 | DHSSSSYDYQYGLAV |
| | (Combined) | |
| 30 | HCDR1 | DYAIS |
| | (Kabat) | |
| 31 | HCDR2 | GIIPISGTANYAQKFQG |
| | (Kabat) | |
| 32 | HCDR3 | DHSSSSYDYQYGLAV |
| | (Kabat) | |
| 33 | HCDR1 | GGTFSDY |
| | (Chothia) | |
| 34 | HCDR2 | IPISGT |
| | (Chothia) | |
| 35 | HCDR3 | DHSSSSYDYQYGLAV |
| | (Chothia) | |
| 36 | VH | QVQLVQSGAEVKKPGSSVKVSCKASGGTFSDYAISWVRQAPGQ |
| | | GLEWMGGIIPISGTANYAQKFQGRVTITADESTSTAYMELSSLRS |
| | | EDTAVYYCARDHSSSSYDYQYGLAVWGQGTLVTVSS |
| 37 | VH DNA | CAGGTGCAATTGGTGCAGAGCGGTGCCGAAGTGAAAAAACCG |
| | | GGCAGCAGCGTGAAAGTTAGCTGCAAAGCATCCGGAGGGAC |
| | | GTTTTCTGACTACGCTATCTCTTGGGTGCGCCAGGCCCCGGG |
| | | CCAGGGCCTCGAGTGGATGGGCGGTATCATCCCGATCTCTG |
| | | GCACTGCGAACTACGCCCAGAAATTTCAGGGCCGGGTGACCA |
| | | TTACCGCCGATGAAAGCACCAGCACCGCCTATATGGAACTGA |
| | | GCAGCCTGCGCAGCGAAGATACGGCCGTGTATTATTGCGCGC |
| | | GTGACCATTCTTCTTCTTCTTACGACTACCAGTACGGTCTGGC |
| | | TGTTTGGGGCCAAGGCACCCTGGTGACTGTTAGCTCA |
| 38 | Heavy Chain | QVQLVGSGAEVKKPGSSVKVSCKASGGTFSDYAISVWRQAPGQ |
| | | GLEWMGGIIPISGTANYAQKFQGRVTITADESTSTAYMELSSLRS |
| | | EDTAVYYCARDHSSSSYDYQYGLAVWGQGTLVTVSSASTKGPS |
| | | VFPLAPSSKSTSGGTAALGCLVKDYFPEPVTVSWNSGALTSGVH |
| | | TFPAVLQSSGLYSLSSVVTVPSSSLGTQTYICNVNHKPSNTKVDK |
| | | RVEPKSCDKTHTCPPCPAPELLGGPSVFLFPPKPKDTLMISRTPE |
| | | VTCVVVDVSHEDPEVKFNWYVDGVEVHNAKTKPREEQYASTYR |
| | | VVSVLTVLHQDWLNGKEYKCKVSNKALPAPIEKTISKAKGQPREP |
| | | QVYTLPPSREEMTKNQVSLTCLVKGFYPSDIAVEWESNGQPENN |
| | | YKTTPPVLDSDGSFFLYSKLTVDKSRWQQGNVFSCSVMHEALHN |
| | | HYTQKSLSLSPGK |
| 39 | Heavy Chain | CAGGTGCAATTGGTGCAGAGCGGTGCCGAAGTGAAAAAACCG |
| | DNA | GGCAGCAGCGTGAAAGTTAGCTGCAAAGCATCCGGAGGGAC |
| | | GTTTTCTGACTACGCTATCTCTTGGGTGCGCCAGGCCCCGGG |
| | | CCAGGGCCTCGAGTGGATGGGCGGTATCATCCCGATCTCTG |
| | | GCACTGCGAACTACGCCCAGAAATTTCAGGGCCGGGTGACCA |
| | | TTACCGCCGATGAAAGCACCAGCACCGCCTATATGGAACTGA |
| | | GCAGCCTGCGCAGCGAAGATACGGCCGTGTATTATTGCGCGC |
| | | GTGACCATTCTTCTTCTTCTTACGACTACCAGTACGGTCTGGC |
| | | TGTTTGGGGCCAAGGCACCCTGGTGACTGTTAGCTCAGCCTC |
| | | CACCAAGGGTCCATCGGTCTTCCCCCTGGCACCCTCCTCCAA |
| | | GAGCACCTCTGGGGGCACAGCGGCCCTGGGCTGCCTGGTCA |
| | | AGGACTACTTCCCCGAACCGGTGACGGTGTCGTGGAACTCAG |
| | | GCGCCCTGACCAGCGGCGTGCACACCTTCCCGGCTGTCCTA |
| | | CAGTCCTCAGGACTCTACTCCCTCAGCAGCGTGGTGACCGTG |
| | | CCCTCCAGCAGCTTGGGCACCCAGACCTACATCTGCAACGTG |
| | | AATCACAAGCCCAGCAACACCAAGGTGGACAAGAGAGTTGAG |
| | | CCCAAATCTTGTGACAAAACTCACACATGCCCACCGTGCCCA |
| | | GCACCTGAACTCCTGGGGGGACCGTCAGTCTTCCTCTTCCCC |
| | | CCAAAACCCAAGGACACCCTCATGATCTCCCGGACCCCTGAG |
| | | GTCACATGCGTGGTGGTGGACGTGAGCCACGAAGACCCTGA |
| | | GGTCAAGTTCAACTGGTACGTGGACGGCGTGGAGGTGCATAA |
| | | TGCCAAGACAAAGCCGCGGGAGGAGCAGTACGCCAGCACGT |
| | | ACCGGGTGGTCAGCGTCCTCACCGTCCTGCACCAGGACTGG |
| | | CTGAATGGCAAGGAGTACAAGTGCAAGGTCTCCAACAAAGCC |
| | | CTCCCAGCCCCCATCGAGAAAACCATCTCCAAAGCCAAAGGG |
| | | CAGCCCCGAGAACCACAGGTGTACACCCTGCCCCCATCCCG |
| | | GGAGGAGATGACCAAGAACCAGGTCAGCCTGACCTGCCTGG |
| | | TCAAAGGCTTCTATCCCAGCGACATCGCCGTGGAGTGGGAGA |
| | | GCAATGGGCAGCCGGAGAACAACTACAAGACCACGCCTCCC |
| | | GTGCTGGACTCCGACGGCTCCTTCTTCCTCTACAGCAAGCTC |
| | | ACCGTGGACAAGAGCAGGTGGCAGCAGGGGAACGTCTTCTC |
| | | ATGCTCCGTGATGCATGAGGCTCTGCACAACCACTACACGCA |
| | | GAAGAGCCTCTCCCTGTCTCCGGGTAAA |
| 40 | LCDR1 | SGDKLGDYYVH |
| | (Combined) | |
| 41 | LCDR2 | QDSKRPS |
| | (Combined) | |
| 42 | LCDR3 | GATDLSPWSIV |
| | (Combined) | |
| 43 | LCDR1 | SGDKLGDYYVH |
| | (Kabat) | |
| 44 | LCDR2 | QDSKRPS |
| | (Kabat) | |
| 45 | LCDR3 | GATDLSPWSIV |
| | (Kabat) | |
| 46 | LCDR1 | DKLGDYY |
| | (Chothia) | |
| 47 | LCDR2 | QDS |
| | (Chothia) | |
| 48 | LCDR3 | TDLSPWSI |
| | (Chothia) | |
| 49 | VL | DIELTQPPSVSVSPGETASITCSGDKLGDYYVHVWQQKPGQAPV |
| | | LVIYQDSKRPSGIPERFSGSNSGNTATLTISGTQAEDEADYYCGAT |
| | | DLSPWSIVFGGGTKLTVL |
| 50 | VH DNA | GATATCGAACTGACCCAGCCGCCGAGCGTGAGCGTGAGCCC |
| | | GGGCGAGACCGCGAGCATTACCTGTAGCGGCGATAAACTGG |
| | | GTGACTACTACGTTCATTGGTACCAGCAGAAACCGGGCCAGG |
| | | CGCCGGTGCTGGTGATCTACCAGGACTCTAAACGTCCGAGCG |
| | | GCATCCCGGAACGTTTTAGCGGATCCAACAGCGGCAACACCG |
| | | CGACCCTGACCATTAGCGGCACCCAGGCGGAAGACGAAGCG |
| | | GATTATTACTGCGGTGCTACTGACCTGTCTCCGTGGTCTATCG |
| | | TGTTTGGCGGCGGCACGAAGTTAACCGTCCTA |
| 51 | Light Chain | DIELTQPPSVSVSPGETASITCSGDKLGDYYVHWYQQKPGQAPV |
| | | LVIYQDSKRPSGIPERFSGSNSGNTATLTISGTQAEDEADYYCGA |
| | | TDLSPWSIVFGGGTKLTVLGQPKAAPSVTLFPPSSEELQANKATL |
| | | VCLISDFYPGAVTVAWKADSSPVKAGVETTTPSKQSNNKYAASS |
| | | YLSLTPEQWKSMRSYSCQVTHEGSTVEKTVAPTECS |
| 52 | Light Chain | GATATCGAACTGACCCAGCCGCCGAGCGTGAGCGTGAGCCC |
| | DNA | GGGCGAGACCGCGAGCATTACCTGTAGCGGCGATAAACTGG |
| | | GTGACTACTACGTTCATTGGTACCAGCAGAAACCGGGCCAGG |
| | | CGCCGGTGCTGGTGATCTACCAGGACTCTAAACGTCCGAGCG |
| | | GCATCCCGGAACGTTTTAGCGGATCCAACAGCGGCAACACCG |
| | | CGACCCTGACCATTAGCGGCACCCAGGCGGAAGACGAAGCG |
| | | GATTATTACTGCGGTGCTACTGACCTGTCTCCGTGGTCTATCG |
| | | TGTTTGGCGGCGGCACGAAGTTAACCGTCCTAGGTCAGCCCA |
| | | AGGCTGCCCCCTCGGTCACTCTGTTCCCGCCCTCCTCTGAGG |
| | | AGCTTCAAGCCAACAAGGCCACACTGGTGTGTCTCATAAGTG |
| | | ACTTCTACCCGGGAGCCGTGACAGTGGCCTGGAAGGCAGATA |
| | | GCAGCCCCGTCAAGGCGGGAGTGGAGACCACCACACCCTCC |
| | | AAACAAAGCAACAACAAGTACGCGGCCAGCAGCTATCTGAGC |
| | | CTGACGCCTGAGCAGTGGAAGTCCCACAGAAGCTACAGCTGC |
| | | CAGGTCACGCATGAAGGGAGCACCGTGGAGAAGACAGTGGCC |
| | | CCTACAGAATGTTCA |

Antibody 003

| | | |
|---|---|---|
| 53 | HCDR1 | GGTFSSYAIS |
| | (Combined) | |
| 54 | HCDR2 | GIIPVLGTANYAQKFQG |
| | (Combined) | |
| 55 | HCDR3 | VPTDYFDY |
| | (Combined) | |
| 56 | HCDR1 | SYAIS |
| | (Kabat) | |
| 57 | HCDR2 | GIIPVLGTANYAQKFQG |
| | (Kabat) | |
| 58 | HCDR3 | VPTDYFDY |
| | (Kabat) | |
| 59 | HCDR1 | GGTFSSY |
| | (Chothia) | |
| 60 | HCDR2 | IPVLGT |
| | (Chothia) | |
| 61 | HCDR3 | VPTDYFDY |
| | (Chothia) | |
| 62 | VH | QVQLVQSGAEVKKPGSSVKVSCKASGGTFSSYAISVWRQAPGQ |
| | | GLEWMGGIIPVLGTANYAQKFQGRVTITADESTSTAYMELSSLRS |
| | | EDTAVYYCARVPTDYFDYWGQGTLVTVSS |
| 63 | VH DNA | CAGGTGCAGCTGGTGCAGTCAGGCGCCGAAGTGAAGAAACC |
| | | CGGCTCTAGCGTGAAAGTCAGCTGTAAAGCTAGTGGCGGCAC |
| | | CTTCTCTAGCTACGCTATTAGCTGGGTCAGACAGGCCCCAGG |
| | | TCAGGGCCTGGAGTGGATGGGCGGAATTATCCCCGTGCTGG |
| | | GCACCGCTAACTACGCTCAGAAATTTCAGGGTAGAGTGACTAT |
| | | CACCGCCGACGAGTCTACTAGCACCGCCTATATGGAACTGTC |
| | | TAGCCTGAGATCAGAGGACACCGCCGTCTACTACTGCGCTAG |
| | | AGTGCCTACCGACTACTTCGACTACTGGGGTCAGGGCACCCTG |
| | | GTCACCGTGTCTAGC |
| 64 | Heavy Chain | QVQLVQSGAEVKKPGSSVKVSCKASGGTFSSYAISWVRQAPGQ |
| | | GLEWMGGIIPVLGTANYAQKFQGRVTITADESTSTAYMELSSLRS |
| | | EDTAVYYCARVPTDYFDYWGQGTLVTVSSASTKGPSVFPLAPSS |
| | | KSTSGGTAALGCLVKDYFPEPVTVSWNSGALTSGVHTFPAVLQS |
| | | SGLYSLSSVVTVPSSSLGTQTYICNVNHKPSNTKVDKRVEPKSCD |
| | | KTHTCPPCPAPELLGGPSVFLFPPKPKDTLMISRTPEVTCVVVDV |
| | | SHEDPEVKFNWYVDGVEVHNAKTKPREEQYNSTYRVVSVLTVLH |
| | | QDWLNGKEYKCKVSNKALPAPIEKTISKAKGQPREPQVYTLPPSR |
| | | EEMTKNGVSLTCLVKGFYPSDIAVEWESNGQPENNYKTTPPVLD |
| | | SDGSFFLYSKLTVDKSRWQQGNVFSCSVMHEALHNHYTQKSLSL |
| | | SPGK |
| 65 | Heavy Chain | CAGGTGCAGCTGGTGCAGTCAGGCGCCGAAGTGAAGAAACC |
| | DNA | CGGCTCTAGCGTGAAAGTCAGCTGTAAAGCTAGTGGCGGCAC |
| | | CTTCTCTAGCTACGCTATTAGCTGGGTCAGACAGGCCCCAGG |
| | | TCAGGGCCTGGAGTGGATGGGCGGAATTATCCCCGTGCTGG |
| | | GCACCGCTAACTACGCTCAGAAATTTCAGGGTAGAGTGACTAT |
| | | CACCGCCGACGAGTCTACTAGCACCGCCTATATGGAACTGTC |
| | | TAGCCTGAGATCAGAGGACACCGCCGTCTACTACTGCGCTAG |
| | | AGTGCCTACCGACTACTTCGACTACTGGGGTCAGGGCACCCT |
| | | GGTCACCGTGTCTAGCGCTAGCACTAAGGGCCCAAGTGTGTT |
| | | TCCCCTGGCCCCCAGCAGCAAGTCTACTTCCGGCGGAACTGC |
| | | TGCCCTGGGTTGCCTGGTGAAGGACTACTTCCCCGAGCCCGT |
| | | GACAGTGTCCTGGAACTCTGGGGCTCTGACTTCCGGCGTGCA |
| | | CACCTTCCCCGCCGTGCTGCAGAGCAGCGGCCTGTACAGCCT |
| | | GAGCAGCGTGGTGACAGTGCCCTCCAGCTCTCTGGGAACCCA |
| | | GACCTATATCTGCAACGTGAACCACAAGCCCAGCAACACCAA |
| | | GGTGGACAAGAGAGTGGAGCCCAAGAGCTGCGACAAGACCC |
| | | ACACCTGCCCCCCCTGCCCAGCTCCAGAACTGCTGGGAGGG |
| | | CCTTCCGTGTTCCTGTTCCCCCCCAAGCCCAAGGACACCCTG |
| | | ATGATCAGCAGGACCCCCGAGGTGACCTGCGTGGTGGTGGA |
| | | CGTGTCCCACGAGGACCCAGAGGTGAAGTTCAACTGGTACGT |
| | | GGACGGCGTGGAGGTGCACAACGCCAAGACCAAGCCCAGAG |
| | | AGGAGCAGTACAACAGCACCTACAGGGTGGTGTCCGTGCTGA |
| | | CCGTGCTGCACCAGGACTGGCTGAACGGCAAAGAATACAAGT |
| | | GCAAAGTCTCCAACAAGGCCCTGCCAGCCCCAATCGAAAAGA |
| | | CAATCAGCAAGGCCAAGGGCCAGCCACGGGAGCCCCAGGTG |
| | | TACACCCTGCCCCCCAGCCGGGAGGAGATGACCAAGAACCAG |
| | | GTGTCCCTGACCTGTCTGGTGAAGGGCTTCTACCCCAGCGAT |
| | | ATCGCCGTGGAGTGGGAGAGCAACGGCCAGCCCGAGAACAA |
| | | CTACAAGACCACCCCCCCAGTGCTGGACAGCGACGGCAGCTT |
| | | CTTCCTGTACAGCAAGCTGACCGTGGACAAGTCCAGGTGGCA |
| | | GCAGGGCAACGTGTTCAGCTGCAGCGTGATGCACGAGGCCCT |
| | | GCACAACCACTACACCCAGAAGTCCCTGAGCCTGAGCCCCGGC |
| | | AAG |
| 66 | LCDR1 | SGDNLGSKYVH |
| | (Combined) | |
| 67 | LCDR2 | DDNKRPS |
| | (Combined) | |
| 68 | LCDR3 | QSWTLGNWV |
| | (Combined) | |
| 69 | LCDR1 | SGDNLGSKYVH |
| | (Kabat) | |
| 70 | LCDR2 | DDNKRPS |
| | (Kabat) | |
| 71 | LCDR3 | QSWTLGNWV |
| | (Kabat) | |
| 72 | LCDR1 | DNLGSKY |
| | (Chothia) | |
| 73 | LCDR2 | DDN |
| | (Chothia) | |
| 74 | LCDR3 | WTLGNW |
| | (Chothia) | |
| 75 | VL | DIELTQPPSVSVSPGQTASITCSGDNLGSKYVHWYGQKPGQAPV |
| | | LVIYDDNKRPSGIPERFSGSNSGNTATLTISGTQAEDEADYYCQS |
| | | W |
| | | TLGNVWFGGGTKLTVL |
| 76 | VH DNA | GATATCGAGCTGACTCAGCCCCCTAGCGTCAGCGTCAGCCCT |
| | | GGTCAGACCGCCTCTATCACCTGTAGCGGCGATAACCTGGGC |
| | | TCTAAATACGTGCACTGGTATCAGCAGAAGCCCGGTCAGGCC |
| | | CCCGTGCTGGTGATCTACGACGATAACAAGCGGCCTAGCGGA |
| | | ATCCCCGAGCGGTTTAGCGGCTCTAATAGCGGTAACACCGCT |
| | | ACCCTGACTATTAGCGGCACTCAGGCCGAGGACGAGGCCGAC |
| | | TACTACTGTCAGTCCTGGACCCTGGGCAACTGGGTGTTCGGC |
| | | GGAGGCACTAAGCTGACCGTGCTG |
| 77 | Light Chain | DIELTQPPSVSVSPGQTASITCSGDNLGSKYVHWVQQKPGGAPV |
| | | LVIYDDNKRPSGIPERFSGSNSGNTATLTISGTQAEDEADYYCGS |
| | | WTLGNWVFGGGTKLTVLGQPKAAPSVTLFPPSSEELQANKATLV |
| | | CLISDFYPGAVTVAWKADSSPVKAGVETTTPSKQSNNKYAASSY |
| | | LSLTPEQWKSHRSYSCQVTHEGSTVEKTVAPTECS |
| 78 | Light Chain | GATATCGAGCTGACTCAGCCCCCTAGCGTCAGCGTCAGCCCT |
| | DNA | GGTCAGACCGCCTCTATCACCTGTAGCGGCGATAACCTGGGC |
| | | TCTAAATACGTGCACTGGTATCAGCAGAAGCCCGGTCAGGCC |
| | | CCCGTGCTGGTGATCTACGACGATAACAAGCGGCCTAGCGGA |
| | | ATCCCCGAGCGGTTTAGCGGCTCTAATAGCGGTAACACCGCT |
| | | ACCCTGACTATTAGCGGCACTCAGGCCGAGGACGAGGCCGAC |
| | | TACTACTGTCAGTCCTGGACCCTGGGCAACTGGGTGTTCGGC |
| | | GGAGGCACTAAGCTGACCGTGCTGGGTCAGCCTAAGGCTGCC |
| | | CCCAGCGTGACCCTGTTCCCCCCCAGCAGCGAGGAGCTGCA |
| | | GGCCAACAAGGCCACCCTGGTGTGCCTGATCAGCGACTTCTA |
| | | CCCAGGCGCCGTGACCGTGGCCTGGAAGGCCGACAGCAGCC |
| | | CCGTGAAGGCCGGCGTGGAGACCACCACCCCCAGCAAGCAG |
| | | AGCAACAACAAGTACGCCGCCAGCAGCTACCTGAGCCTGACC |
| | | CCCGAGCAGTGGAAGAGCCACAGGTCCTACAGCTGCCAGGT |
| | | GACCCACGAGGGCAGCACCGTGGAAAAGACCGTGGCCCCAAC |
| | | CGAGTGCAGC |

Antibody 004

| | | |
|---|---|---|
| 79 | HCDR1 | GGTFSSYAIS |
| | (Combined) | |
| 80 | HCDR2 | GIIPVLGTANYAQKFQG |
| | (Combined) | |
| 81 | HCDR3 | VPTDYFDY |
| | (Combined) | |
| 82 | HCDR1 | SYAIS |
| | (Kabat) | |
| 83 | HCDR2 | GIIPVLGTANYAQKFQG |
| | (Kabat) | |
| 84 | HCDR3 | VPTDYFDY |
| | (Kabat) | |
| 85 | HCDR1 | GGTFSSY |
| | (Chothia) | |
| 86 | HCDR2 | IPVLGT |
| | (Chothia) | |
| 87 | HCDR3 | VPTDYFDY |
| | (Chothia) | |
| 88 | VH | QVQLVQSGAEVKKPGSSVKVSCKASGGTFSSYAISWVRQAPGQ |
| | | GLEWMGGIIPVLGTANYAQKFQGRVTITADESTSTAYMELSSLRS |
| | | EDTAVYYCARVPTDYFDYWGQGTLVTVSS |
| 89 | VH DNA | CAGGTGCAATTGGTGCAGAGCGGTGCCGAAGTGAAAAAACCG |
| | | GGCAGCAGCGTGAAAGTTAGCTGCAAAGCATCCGGAGGGAC |
| | | GTTTTCTTCTTACGCTATCTCTTGGGTGCGCCAGGCCCCGGG |
| | | CCAGGGCCTCGAGTGGATGGGCGGTATCATCCCGGTTCTGG |
| | | GCACTGCGAACTACGCCCAGAAATTTCAGGGCCGGGTGACCA |
| | | TTACCGCCGATGAAAGCACCAGCACCGCCTATATGGAACTGA |
| | | GCAGCCTGCGCAGCGAAGATACGGCCGTGTATTATTGCGCGC |
| | | GTGTTCCGACTGACTACTTCGATTACTGGGGCCAAGGCACCCT |
| | | GGTGACTGTTAGCTCA |
| 90 | Heavy Chain | QVQLVQSGAEVKKPGSSVKVSCKASGGTFSSYAISWVRQAPGQ |
| | | GLEWMGGIIPVLGTANYAQKFQGRVTITADESTSTAYMELSSLRS |
| | | EDTAVYYCARVPTDYFDYWGQGTLVTVSSASTKGPSVFPLAPSS |
| | | KSTSGGTAALGCLVKDYFPEPVTVSWNSGALTSGVHTFPAVLQS |
| | | SGLYSLSSWTVPSSSLGTQTYICNVNHKPSNTKVDKRVEPKSCD |
| | | KTHTCPPCPAPELLGGPSVFLFPPKPKDTLMISRTPEVTCVVVDV |
| | | SHEDPEVKFNWYVDGVEVHNAKTKPREEQYASTYRVVSVLTVLH |
| | | QDWLNGKEYKCKVSNKALPAPIEKTISKAKGQPREPQVYTLPPSR |
| | | EEMTKNQVSLTCLVKGFYPSDIAVEWESNGQPENNYKTTPPVLD |
| | | SDGSFFLYSKLTVDKSRWQQGNVFSCSVMHEALHNHYTQKSLSL |
| | | SPGK |
| 91 | Heavy Chain | CAGGTGCAATTGGTGCAGAGCGGTGCCGAAGTGAAAAAACCG |
| | DNA | GGCAGCAGCGTGAAAGTTAGCTGCAAAGCATCCGGAGGGAC |
| | | GTTTTCTTCTTACGCTATCTCTTGGGTGCGCCAGGCCCCGGG |
| | | CCAGGGCCTCGAGTGGATGGGCGGTATCATCCCGGTTCTGG |
| | | GCACTGCGAACTACGCCCAGAAATTTCAGGGCCGGGTGACCA |
| | | TTACCGCCGATGAAAGCACCAGCACCGCCTATATGGAACTGA |
| | | GCAGCCTGCGCAGCGAAGATACGGCCGTGTATTATTGCGCGC |
| | | GTGTTCCGACTGACTACTTCGATTACTGGGGCCAAGGCACCC |
| | | TGGTGACTGTTAGCTCAGCCTCCACCAAGGGTCCATCGGTCTT |
| | | CCCCCTGGCACCCTCCTCCAAGAGCACCTCTGGGGGCACAGC |
| | | GGCCCTGGGCTGCCTGGTCAAGGACTACTTCCCCGAACCGGT |
| | | GACGGTGTCGTGGAACTCAGGCGCCCTGACCAGCGGCGTGC |
| | | ACACCTTCCCGGCTGTCCTACAGTCCTCAGGACTCTACTCCCT |
| | | CAGCAGCGTGGTGACCGTGCCCTCCAGCAGCTTGGGCACCC |
| | | AGACCTACATCTGCAACGTGAATCACAAGCCCAGCAACACCAA |
| | | GGTGGACAAGAGAGTTGAGCCCAAATCTTGTGACAAAACTCAC |
| | | ACATGCCCACCGTGCCCAGCACCTGAACTCCTGGGGGGACC |
| | | GTCAGTCTTCCTCTTCCCCCCAAAACCCAAGGACACCCTCATG |
| | | ATCTCCCGGACCCCTGAGGTCACATGCGTGGTGGTGGACGTG |
| | | AGCCACGAAGACCCTGAGGTCAAGTTCAACTGGTACGTGGAC |
| | | GGCGTGGAGGTGCATAATGCCAAGACAAAGCCGCGGGAGGA |
| | | GCAGTACGCCAGCACGTACCGGGTGGTCAGCGTCCTCACCGT |
| | | CCTGCACCAGGACTGGCTGAATGGCAAGGAGTACAAGTGCAA |
| | | GGTCTCCAACAAAGCCCTCCCAGCCCCCATCGAGAAAACCAT |
| | | CTCCAAAGCCAAAGGGCAGCCCCGAGAACCACAGGTGTACAC |
| | | CCTGCCCCCATCCCGGGAGGAGATGACCAAGAACCAGGTCAG |
| | | CCTGACCTGCCTGGTCAAAGGCTTCTATCCCAGCGACATCGC |
| | | CGTGGAGTGGGAGAGCAATGGGCAGCCGGAGAACAACTACA |
| | | AGACCACGCCTCCCGTGCTGGACTCCGACGGCTCCTTCTTCC |
| | | TCTACAGCAAGCTCACCGTGGACAAGAGCAGGTGGCAGCAGG |
| | | GGAACGTCTTCTCATGCTCCGTGATGCATGAGGCTCTGCACAA |
| | | CCACTACACGCAGAAGAGCCTCTCCCTGTCTCCGGGTAAA |
| 92 | LCDR1 | SGDNLGSKYVH |
| | (Combined) | |
| 93 | LCDR2 | DDNKRPS |
| | (Combined) | |
| 94 | LCDR3 | QSWTLGNWV |
| | (Combined) | |
| 95 | LCDR1 | SGDNLGSKYVH |
| | (Kabat) | |
| 96 | LCDR2 | DDNKRPS |
| | (Kabat) | |
| 97 | LCDR3 | QSWTLGNWV |
| | (Kabat) | |
| 98 | LCDR1 | DNLGSKY |
| | (Chothia) | |
| 99 | LCDR2 | DDN |
| | (Chothia) | |
| 100 | LCDR3 | WTLGNW |
| | (Chothia) | |
| 101 | VL | DIELTQPPSVSVSPGQTASITCSGDNLGSKYVHWYQQKPGQAPV |
| | | LVIYDDNKRPSGIPERFSGSNSGNTATLTISGTQAEDEADYYCQS |
| | | W |
| | | TLGNWVFGGGTKLTVL |
| 102 | VH DNA | GATATCGAACTGACCCAGCCGCCGAGCGTGAGCGTGAGCCC |
| | | GGGCCAGACCGCGAGCATTACCTGTAGCGGCGATAACCTGG |
| | | GTTCTAAATACGTTCATTGGTACCAGCAGAAACCGGGCCAGG |
| | | CGCCGGTGCTGGTGATCTACGACGACAACAAACGTCCGAGCG |
| | | GCATCCCGGAACGTTTTAGCGGATCCAACAGCGGCAACACCG |
| | | CGACCCTGACCATTAGCGGCACCCAGGCGGAAGACGAAGCG |
| | | GATTATTACTGCCAGTCTTGGACTCTGGGTAACTGGGTGTTTG |
| | | GCGGCGGCACGAAGTTAACCGTCCTA |
| 103 | Light Chain | DIELTQPPSVSVSPGQTASITCSGDNLGSKYVHWYQQKPGQAPV |
| | | LVIYDDNKRPSGIPERFSGSNSGNTATLTISGTQAEDEADYYCQS |
| | | WTLGNWVFGGGTKLTVLGQPKAAPSVTLFPPSSEELQANKATLV |
| | | CLISDFYPGAVTVAWKADSSPVKAGVETTTPSKQSNNKYAASSY |
| | | LSLTPEQWKSHRSYSCQVTHEGSTVEKTVAPTECS |
| 104 | Light Chain | GATATCGAACTGACCCAGCCGCCGAGCGTGAGCGTGAGCCC |
| | DNA | GGGCCAGACCGCGAGCATTACCTGTAGCGGCGATAACCTGG |
| | | GTTCTAAATACGTTCATTGGTACCAGCAGAAACCGGGCCAGG |
| | | CGCCGGTGCTGGTGATCTACGACGACAACAAACGTCCGAGCG |
| | | GCATCCCGGAACGTTTTAGCGGATCCAACAGCGGCAACACCG |
| | | CGACCCTGACCATTAGCGGCACCCAGGCGGAAGACGAAGCG |
| | | GATTATTACTGCCAGTCTTGGACTCTGGGTAACTGGGTGTTTG |
| | | GCGGCGGCACGAAGTTAACCGTCCTAGGTCAGCCCAAGGCTG |
| | | CCCCCTCGGTCACTCTGTTCCCGCCCTCCTCTGAGGAGCTTC |
| | | AAGCCAACAAGGCCACACTGGTGTGTCTCATAAGTGACTTCTA |
| | | CCCGGGAGCCGTGACAGTGGCCTGGAAGGCAGATAGCAGCC |
| | | CCGTCAAGGCGGGAGTGGAGACCACCACACCCTCCAAACAAA |
| | | GCAACAACAAGTACGCGGCCAGCAGCTATCTGAGCCTGACGC |
| | | CTGAGCAGTGGAAGTCCCACAGAAGCTACAGCTGCCAGGTCA |
| | | CGCATGAAGGGAGCACCGTGGAGAAGACAGTGGCCCCTACAG |
| | | AATGTTCA |

Antibody 005

| | | |
|---|---|---|
| 105 | HCDR1 | GGTFSDNAIS |
| | (Combined) | |
| 106 | HCDR2 | GINPDFGWANYAQKFQG |
| | (Combined) | |
| 107 | HCDR3 | DSSGMGY |
| | (Combined) | |
| 108 | HCDR1 | DNAIS |
| | (Kabat) | |
| 109 | HCDR2 | GINPDFGWANYAQKFQG |
| | (Kabat) | |
| 110 | HCDR3 | DSSGMGY |
| | (Kabat) | |
| 111 | HCDR1 | GGTFSDN |
| | (Chothia) | |
| 112 | HCDR2 | NPDFGW |
| | (Chothia) | |
| 113 | HCDR3 | DSSGMGY |
| | (Chothia) | |
| 114 | VH | QVQLVQSGAEVKKPGSSVKVSCKASGGTFSDNAISWVRQAPGQ |
| | | GLEWMGGINPDFGWANYAQKFQGRVTITADESTSTAYMELSSLR |
| | | SEDTAVYYCARDSSGMGYWGQGTLVTVSS |
| 115 | VH DNA | CAGGTGCAGCTGGTGCAGTCAGGCGCCGAAGTGAAGAAACC |
| | | CGGCTCTAGCGTGAAAGTCAGCTGTAAAGCTAGTGGCGGCAC |
| | | CTTTAGCGATAACGCTATTAGCTGGGTCAGACAGGCCCCAGG |
| | | TCAGGGCCTGGAGTGGATGGGCGGGATTAACCCCGACTTCG |
| | | GCTGGGCTAACTACGCTCAGAAATTTCAGGGTAGAGTGACTAT |
| | | CACCGCCGACGAGTCTACTAGCACCGCCTATATGGAACTGTC |
| | | TAGCCTGAGATCAGAGGACACCGCCGTCTACTACTGCGCTAG |
| | | GGACTCTAGCGGAATGGGCTACTGGGGTCAGGGCACCCTGGTC |
| | | ACCGTGTCTAGC |
| 116 | Heavy Chain | QVQLVQSGAEVKKPGSSVKVSCKASGGTFSDNAISVWRQAPGQ |
| | | GLEWMGGINPDFGWANYAQKFGGRVTITADESTSTAYMELSSLR |
| | | SEDTAVYYCARDSSGMGYWGQGTLVTVSSASTKGPSVFPLAPS |
| | | SKSTSGGTAALGCLVKDYFPEPVTVSWNSGALTSGVHTFPAVLQ |
| | | SSGLYSLSSVVTVPSSSLGTQTYICNVNHKPSNTKVDKRVEPKSC |
| | | DKTHTCPPCPAPELLGGPSVFLFPPKPKDTLMISRTPEVTCVVVD |
| | | VSHEDPEVKFNWYVDGVEVHNAKTKPREEQYNSTYRVVSVLTVL |
| | | HQDWLNGKEYKCKVSNKALPAPIEKTISKAKGQPREPQVYTLPPS |
| | | REEMTKNQVSLTCLVKGFYPSDIAVEWESNGQPENNYKTTPPVL |
| | | DSDGSFFLYSKLTVDKSRWQQGNVFSCSVMHEALHNHYTQKSL |
| | | SL |
| | | SPGK |
| 117 | Heavy Chain | CAGGTGCAGCTGGTGCAGTCAGGCGCCGAAGTGAAGAAACC |
| | DNA | CGGCTCTAGCGTGAAAGTCAGCTGTAAAGCTAGTGGCGGCAC |
| | | CTTTAGCGATAACGCTATTAGCTGGGTCAGACAGGCCCCAGG |
| | | TCAGGGCCTGGAGTGGATGGGCGGGATTAACCCCGACTTCG |
| | | GCTGGGCTAACTACGCTCAGAAATTTCAGGGTAGAGTGACTAT |
| | | CACCGCCGACGAGTCTACTAGCACCGCCTATATGGAACTGTC |
| | | TAGCCTGAGATCAGAGGACACCGCCGTCTACTACTGCGCTAG |
| | | GGACTCTAGCGGAATGGGCTACTGGGGTCAGGGCACCCTGG |
| | | TCACCGTGTCTAGCGCTAGCACTAAGGGCCCAAGTGTGTTTC |
| | | CCCTGGCCCCCAGCAGCAAGTCTACTTCCGGCGGAACTGCTG |
| | | CCCTGGGTTGCCTGGTGAAGGACTACTTCCCCGAGCCCGTGA |
| | | CAGTGTCCTGGAACTCTGGGGCTCTGACTTCCGGCGTGCACA |
| | | CCTTCCCCGCCGTGCTGCAGAGCAGCGGCCTGTACAGCCTGA |
| | | GCAGCGTGGTGACAGTGCCCTCCAGCTCTCTGGGAACCCAGA |
| | | CCTATATCTGCAACGTGAACCACAAGCCCAGCAACACCAAGGT |
| | | GGACAAGAGAGTGGAGCCCAAGAGCTGCGACAAGACCCACA |
| | | CCTGCCCCCCCTGCCCAGCTCCAGAACTGCTGGGAGGGCCTT |
| | | CCGTGTTCCTGTTCCCCCCCAAGCCCAAGGACACCCTGATGA |
| | | TCAGCAGGACCCCCGAGGTGACCTGCGTGGTGGTGGACGTG |
| | | TCCCACGAGGACCCAGAGGTGAAGTTCAACTGGTACGTGGAC |
| | | GGCGTGGAGGTGCACAACGCCAAGACCAAGCCCAGAGAGGA |
| | | GCAGTACAACAGCACCTACAGGGTGGTGTCCGTGCTGACCGT |
| | | GCTGCACCAGGACTGGCTGAACGGCAAAGAATACAAGTGCAA |
| | | AGTCTCCAACAAGGCCCTGCCAGCCCCAATCGAAAAGACAAT |
| | | CAGCAAGGCCAAGGGCCAGCCACGGGAGCCCCAGGTGTACA |
| | | CCCTGCCCCCCAGCCGGGAGGAGATGACCAAGAACCAGGTG |
| | | TCCCTGACCTGTCTGGTGAAGGGCTTCTACCCCAGCGATATC |
| | | GCCGTGGAGTGGGAGAGCAACGGCCAGCCCGAGAACAACTA |
| | | CAAGACCACCCCCCCAGTGCTGGACAGCGACGGCAGCTTCTT |
| | | CCTGTACAGCAAGCTGACCGTGGACAAGTCCAGGTGGCAGCA |
| | | GGGCAACGTGTTCAGCTGCAGCGTGATGCACGAGGCCCTGCA |
| | | CAACCACTACACCCAGAAGTCCCTGAGCCTGAGCCCCGGCAA |
| | | G |
| 118 | LCDR1 | RASQDISSYLN |
| | (Combined) | |
| 119 | LCDR2 | DASTLQS |
| | (Combined) | |
| 120 | LCDR3 | QQSGHWLSKT |
| | (Combined) | |
| 121 | LCDR1 | RASQDISSYLN |
| | (Kabat) | |
| 122 | LCDR2 | DASTLQS |
| | (Kabat) | |
| 123 | LCDR3 | QQSGHWLSKT |
| | (Kabat) | |
| 124 | LCDR1 | SQDISSY |
| | (Chothia) | |
| 125 | LCDR2 | DAS |
| | (Chothia) | |
| 126 | LCDR3 | SGHWLSK |
| | (Chothia) | |
| 127 | VL | DIQMTGSPSSLSASVGDRVTITCRASQDISSYLNWYQQKPGKAP |
| | | KLLIYDASTLQSGVPSRFSGSGSGTDFTLTISSLQPEDFATYYCQ |
| | | QSG |
| | | HWLSKTFGQGTKVEIK |
| 128 | VH DNA | GATATTCAGATGACTCAGTCACCTAGTAGCCTGAGCGCTAGTG |
| | | TGGGCGATAGAGTGACTATCACCTGTAGAGCCTCTCAGGATAT |
| | | CTCTAGCTACCTGAACTGGTATCAGCAGAAGCCCGGTAAAGC |
| | | CCCTAAGCTGCTGATCTACGACGCCTCTACCCTGCAGTCAGG |
| | | CGTGCCCTCTAGGTTTAGCGGTAGCGGTAGTGGCACCGACTT |
| | | CACCCTGACTATCTCTAGCCTGCAGCCCGAGGACTTCGCTAC |
| | | CTACTACTGTCAGCAGTCAGGCCACTGGCTGTCTAAGACCTTC |
| | | GGTCAGGGCACTAAGGTCGAGATTAAG |
| 129 | Light Chain | DIQMTQSPSSLSASVGDRVTITCRASQDISSYLNWYQQKPGKAP |
| | | KLLIYDASTLQSGVPSRFSGSGSGTDFTLTISSLQPEDFATYYCQ |
| | | QSGHWLSKTFGQGTKVEIKRTVAAPSVFIFPPSDEQLKSGTASVV |
| | | CLLNNFYPREAKVQWKVDNALQSGNSQESVTEQDSKDSTYSLS |
| | | STLTLSKADYEKHKVYACEVTHQGLSSPVTKSFNRGEC |
| 130 | Light Chain | GATATTCAGATGACTCAGTCACCTAGTAGCCTGAGCGCTAGTG |
| | DNA | TGGGCGATAGAGTGACTATCACCTGTAGAGCCTCTCAGGATAT |
| | | CTCTAGCTACCTGAACTGGTATCAGCAGAAGCCCGGTAAAGC |
| | | CCCTAAGCTGCTGATCTACGACGCCTCTACCCTGCAGTCAGG |
| | | CGTGCCCTCTAGGTTTAGCGGTAGCGGTAGTGGCACCGACTT |
| | | CACCCTGACTATCTCTAGCCTGCAGCCCGAGGACTTCGCTAC |
| | | CTACTACTGTCAGCAGTCAGGCCACTGGCTGTCTAAGACCTTC |
| | | GGTCAGGGCACTAAGGTCGAGATTAAGCGTACGGTGGCCGCT |
| | | CCCAGCGTGTTCATCTTCCCCCCCAGCGACGAGCAGCTGAAG |
| | | AGCGGCACCGCCAGCGTGGTGTGCCTGCTGAACAACTTCTAC |
| | | CCCCGGGAGGCCAAGGTGCAGTGGAAGGTGGACAACGCCCT |
| | | GCAGAGCGGCAACAGCCAGGAGAGCGTCACCGAGCAGGACA |
| | | GCAAGGACTCCACCTACAGCCTGAGCAGCACCCTGACCCTGA |
| | | GCAAGGCCGACTACGAGAAGCATAAGGTGTACGCCTGCGAGG |
| | | TGACCCACCAGGGCCTGTCCAGCCCCGTGACCAAGAGCTTCA |
| | | AC |
| | | AGGGGCGAGTGC |

Antibody 006

| | | |
|---|---|---|
| 131 | HCDR1 | GGTFGDNAIS |
| | (Combined) | |
| 132 | HCDR2 | GINPDFGWANYAQKFQG |
| | (Combined) | |
| 133 | HCDR3 | DSSGMGY |
| | (Combined) | |
| 134 | HCDR1 | DNAIS |
| | (Kabat) | |
| 135 | HCDR2 | GINPDFGWANYAQKFQG |
| | (Kabat) | |
| 136 | HCDR3 | DSSGMGY |
| | (Kabat) | |
| 137 | HCDR1 | GGTFSDN |
| | (Chothia) | |
| 138 | HCDR2 | NPDFGW |
| | (Chothia) | |
| 139 | HCDR3 | DSSGMGY |
| | (Chothia) | |
| 140 | VH | QVQLVQSGAEVKKPGSSVKVSCKASGGTFSDNAISVWRQAPGQ |
| | | GLEWMGGINPDFGWANYAQKFQGRVTITADESTSTAYMELSSLR |
| | | SEDTAVYYCARDSSGMGYWGQGTLVTVSS |
| 141 | VH DNA | CAGGTGCAATTGGTGCAGAGCGGTGCCGAAGTGAAAAAACCG |
| | | GGCAGCAGCGTGAAAGTTAGCTGCAAAGCATCCGGAGGGAC |
| | | GTTTTCTGACAACGCTATCTCTTGGGTGCGCCAGGCCCCGGG |
| | | CCAGGGCCTCGAGTGGATGGGCGGTATCAACCCGGACTTCG |
| | | GCTGGGCGAACTACGCCCAGAAATTTCAGGGCCGGGTGACCA |
| | | TTACCGCCGATGAAAGCACCAGCACCGCCTATATGGAACTGA |
| | | GCAGCCTGCGCAGCGAAGATACGGCCGTGTATTATTGCGCGC |
| | | GTGACTCTTCTGGTATGGGTTACTGGGGCCAAGGCACCCTGG |
| | | TG |
| | | ACTGTTAGCTCA |
| 142 | Heavy Chain | QVQLVQSGAEVKKPGSSVKVSCKASGGTFSDNAISWVRQAPGQ |
| | | GLEWMGGINPDFGWANYAQKFQGRVTITADESTSTAYMELSSLR |
| | | SEDTAVYYCARDSSGMGYWGQGTLVTVSSASTKGPSVFPLAPS |
| | | SKSTSGGTAALGCLVKDYFPEPVTVSWNSGALTSGVHTFPAVLQ |
| | | SSGLYSLSSVVTVPSSSLGTGTYICNVNHKPSNTKVDKRVEPKSC |
| | | DKTHTCPPCPAPELLGGPSVFLFPPKPKDTLMISRTPEVTCVVVD |
| | | VSHEDPEVKFNWYVDGVEVHNAKTKPREEQYASTYRVVSVLTVL |
| | | HQDWLNGKEYKCKVSNKALPAPIEKTISKAKGQPPEPQVYTLPPS |
| | | REEMTKNQVSLTCLVKGFYPSDIAVEWESNGQPENNYKTTPPVL |
| | | DSDGSFFLYSKLTVDKSRWQQGNVFSCSVMHEALHNHYTQKSL |
| | | SL |
| | | SPGK |
| 143 | Heavy Chain | CAGGTGCAATTGGTGCAGAGCGGTGCCGAAGTGAAAAAACCG |
| | DNA | GGCAGCAGCGTGAAAGTTAGCTGCAAAGCATCCGGAGGGAC |
| | | GTTTTCTGACAACGCTATCTCTTGGGTGCGCCAGGCCCCGGG |
| | | CCAGGGCCTCGAGTGGATGGGCGGTATCAACCCGGACTTCG |
| | | GCTGGGCGAACTACGCCCAGAAATTTCAGGGCCGGGTGACCA |
| | | TTACCGCCGATGAAAGCACCAGCACCGCCTATATGGAACTGA |
| | | GCAGCCTGCGCAGCGAAGATACGGCCGTGTATTATTGCGCGC |
| | | GTGACTCTTCTGGTATGGGTTACTGGGGCCAAGGCACCCTGG |
| | | TGACTGTTAGCTCAGCCTCCACCAAGGGTCCATCGGTCTTCCC |
| | | CCTGGCACCCTCCTCCAAGAGCACCTCTGGGGGCACAGCGG |
| | | CCCTGGGCTGCCTGGTCAAGGACTACTTCCCCGAACCGGTGA |
| | | CGGTGTCGTGGAACTCAGGCGCCCTGACCAGCGGCGTGCAC |
| | | ACCTTCCCGGCTGTCCTACAGTCCTCAGGACTCTACTCCCTCA |
| | | GCAGCGTGGTGACCGTGCCCTCCAGCAGCTTGGGCACCCAG |
| | | ACCTACATCTGCAACGTGAATCACAAGCCCAGCAACACCAAG |
| | | GTGGACAAGAGAGTTGAGCCCAAATCTTGTGACAAAACTCACA |
| | | CATGCCCACCGTGCCCAGCACCTGAACTCCTGGGGGGACCGT |
| | | CAGTCTTCCTCTTCCCCCCAAAACCCAAGGACACCCTCATGAT |
| | | CTCCCGGACCCCTGAGGTCACATGCGTGGTGGTGGACGTGA |
| | | GCCACGAAGACCCTGAGGTCAAGTTCAACTGGTACGTGGACG |
| | | GCGTGGAGGTGCATAATGCCAAGACAAAGCCGCGGGAGGAG |
| | | CAGTACGCCAGCACGTACCGGGTGGTCAGCGTCCTCACCGTC |
| | | CTGCACCAGGACTGGCTGAATGGCAAGGAGTACAAGTGCAAG |
| | | GTCTCCAACAAAGCCCTCCCAGCCCCCATCGAGAAAACCATCT |
| | | CCAAAGCCAAAGGGCAGCCCCGAGAACCACAGGTGTACACCC |
| | | TGCCCCCATCCCGGGAGGAGATGACCAAGAACCAGGTCAGCC |
| | | TGACCTGCCTGGTCAAAGGCTTCTATCCCAGCGACATCGCCG |
| | | TGGAGTGGGAGAGCAATGGGCAGCCGGAGAACAACTACAAGA |
| | | CCACGCCTCCCGTGCTGGACTCCGACGGCTCCTTCTTCCTCT |
| | | ACAGCAAGCTCACCGTGGACAAGAGCAGGTGGCAGCAGGGG |
| | | AACGTCTTCTCATGCTCCGTGATGCATGAGGCTCTGCACAACC |
| | | ACTACACGCAGAAGAGCCTCTCCCTGTCTCCGGGTAAA |
| 144 | LCDR1 | RASQDISSYLN |
| | (Combined) | |
| 145 | LCDR2 | DASTLQS |
| | (Combined) | |
| 146 | LCDR3 | QQSGHWLSKT |
| | (Combined) | |
| 147 | LCDR1 | RASQDISSYLN |
| | (Kabat) | |
| 148 | LCDR2 | DASTLQS |
| | (Kabat) | |
| 149 | LCDR3 | QQSGHWLSKT |
| | (Kabat) | |
| 150 | LCDR1 | SQDISSY |
| | (Chothia) | |
| 151 | LCDR2 | DAS |
| | (Chothia) | |
| 152 | LCDR3 | SGHWLSK |
| | (Chothia) | |
| 153 | VL | DIQMTQSPSSLSASVGDRVTITCRASQDISSYLNWYQQKPGKAP |
| | | KLLIYDASTLQSGVPSRFSGSGSGTDFTLTISSLQPEDFATYYCQQS |
| | | GHWLSKTFGQGTKVEIK |
| 154 | VH DNA | GATATCCAGATGACCCAGAGCCCGAGCAGCCTGAGCGCCAGC |
| | | GTGGGCGATCGCGTGACCATTACCTGCAGAGCCAGCCAGGAC |
| | | ATTTCTTCTTACCTGAACTGGTACCAGCAGAAACCGGGCAAAG |
| | | CGCCGAAACTATTAATCTACGACGCTTCTACTCTGCAAAGCGG |
| | | CGTGCCGAGCCGCTTTAGCGGCAGCGGATCCGGCACCGATTT |
| | | CACCCTGACCATTAGCTCTCTGCAACCGGAAGACTTTGCGACC |
| | | TATTATTGCCAGCAGTCTGGTCATTGGCTGTCTAAAACCTTTG |
| | | GCCAGGGCACGAAAGTTGAAATTAAA |
| 155 | Light Chain | DIQMTQSPSSLSASVGDRVTITCRASQDISSYLNWYQQKPGKAP |
| | | KLLIYDASTLQSGVPSRFSGSGSGTDFTLTISSLQPEDFATYYCQ |
| | | QSGHWLSKTFGQGTKVEIKRTVAAPSVFIFPPSDEQLKSGTASVV |
| | | CLLNNFYPREAKVQWKVDNALQSGNSQESVTEQDSKDSTYSLS |
| | | STLTLSKADYEKHKVYACEVTHQGLSSPVTKSFNRGEC |
| 156 | Light Chain | GATATCCAGATGACCCAGAGCCCGAGCAGCCTGAGCGCCAGC |
| | DNA | GTGGGCGATCGCGTGACCATTACCTGCAGAGCCAGCCAGGAC |
| | | ATTTCTTCTTACCTGAACTGGTACCAGCAGAAACCGGGCAAAG |
| | | CGCCGAAACTATTAATCTACGACGCTTCTACTCTGCAAAGCGG |
| | | CGTGCCGAGCCGCTTTAGCGGCAGCGGATCCGGCACCGATTT |
| | | CACCCTGACCATTAGCTCTCTGCAACCGGAAGACTTTGCGACC |
| | | TATTATTGCCAGCAGTCTGGTCATTGGCTGTCTAAAACCTTTG |
| | | GCCAGGGCACGAAAGTTGAAATTAAACGTACGGTGGCCGCTC |
| | | CCAGCGTGTTCATCTTCCCCCCCAGCGACGAGCAGCTGAAGA |
| | | GCGGCACCGCCAGCGTGGTGTGCCTGCTGAACAACTTCTACC |
| | | CCCGGGAGGCCAAGGTGCAGTGGAAGGTGGACAACGCCCTG |
| | | CAGAGCGGCAACAGCCAGGAAAGCGTCACCGAGCAGGACAG |
| | | CAAGGACTCCACCTACAGCCTGAGCAGCACCCTGACCCTGAG |
| | | CAAGGCCGACTACGAGAAGCACAAGGTGTACGCCTGCGAGGT |
| | | GACCCACCAGGGCCTGTCCAGCCCCGTGACCAAGAGCTTCAAC |
| | | CGGGGCGAGTGT |

Antibody 007

| | | |
|---|---|---|
| 157 | HCDR1 | GGTFRDYAIS |
| | (Combined) | |
| 158 | HCDR2 | GIIPAFGTANYAQKFQG |
| | (Combined) | |
| 159 | HCDR3 | EQDPEYGYGGYPYEAMDV |
| | (Combined) | |
| 160 | HCDR1 | DYAIS |
| | (Kabat) | |
| 161 | HCDR2 | GIIPAFGTANYAQKFQG |
| | (Kabat) | |
| 162 | HCDR3 | EQDPEYGYGGYPYEAMDV |
| | (Kabat) | |
| 163 | HCDR1 | GGTFRDY |
| | (Chothia) | |
| 164 | HCDR2 | IPAFGT |
| | (Chothia) | |
| 165 | HCDR3 | EQDPEYGYGGYPYEAMDV |
| | (Chothia) | |
| 166 | VH | QVQLVQSGAEVKKPGSSVKVSCKASGGTFRDAISVWRQAP |
| | | GQGLEWMGGIIPAFGTANYAQKFQGRVTITADESTSTAYMELS |
| | | SLRSEDTAVYYCAREQDPEYGYGGYPYEAMDVWGQGTLVTVSS |
| 167 | VH DNA | CAGGTGCAGCTGGTGCAGTCAGGCGCCGAAGTGAAGAAAC |
| | | CCGGCTCTAGCGTGAAAGTCAGCTGTAAAGCTAGTGGCGG |
| | | CACCTTTAGAGACTACGCTATTAGCTGGGTCAGACAGGCCC |
| | | CAGGTCAGGGCCTGGAGTGGATGGGCGGAATTATCCCCGC |
| | | CTTCGGCACCGCTAACTACGCTCAGAAATTTCAGGGTAGAG |
| | | TGACTATCACCGCCGACGAGTCTACTAGCACCGCCTATATG |
| | | GAACTGTCTAGCCTGAGATCAGAGGACACCGCCGTCTACTA |
| | | CTGCGCTAGAGAGCAGGACCCCGAGTACGGCTACGGCGG |
| | | CTACCCCTACGAGGCTATGGACGTGTGGGGTCAGGGCACCCT |
| | | GGTCACCGTGTCTAGC |
| 168 | Heavy Chain | QVQLVQSGAEVKKPGSSVKVSCKASGGTFRDYAISWVRQAP |
| | | GQGLEWMGGIIPAFGTANYAQKFQGRVTITADESTSTAYMELS |
| | | SLRSEDTAVYYCAREQDPEYGYGGYPYEAMDVWGQGTLVTV |
| | | SSASTKGPSVFPLAPSSKSTSGGTAALGCLVKDYFPEPVTVS |
| | | WNSGALTSGVHTFPAVLQSSGLYSLSSVVTVPSSSLGTQTYIC |
| | | NVNHKPSNTKVDKRVEPKSCDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPSREEMTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPGK |
| 169 | Heavy Chain | CAGGTGCAGCTGGTGCAGTCAGGCGCCGAAGTGAAGAAAC |
| | DNA | CCGGCTCTAGCGTGAAAGTCAGCTGTAAAGCTAGTGGCGG |
| | | CACCTTTAGAGACTACGCTATTAGCTGGGTCAGACAGGCCC |
| | | CAGGTCAGGGCCTGGAGTGGATGGGCGGAATTATCCCCGC |
| | | CTTCGGCACCGCTAACTACGCTCAGAAATTTCAGGGTAGAG |
| | | TGACTATCACCGCCGACGAGTCTACTAGCACCGCCTATATG |
| | | GAACTGTCTAGCCTGAGATCAGAGGACACCGCCGTCTACTA |
| | | CTGCGCTAGAGAGCAGGACCCCGAGTACGGCTACGGCGG |
| | | CTACCCCTACGAGGCTATGGACGTGTGGGGTCAGGGCACC |
| | | CTGGTCACCGTGTCTAGCGCTAGCACTAAGGGCCCAAGTG |
| | | TGTTTCCCCTGGCCCCCAGCAGCAAGTCTACTTCCGGCGG |
| | | AACTGCTGCCCTGGGTTGCCTGGTGAAGGACTACTTCCCC |
| | | GAGCCCGTGACAGTGTCCTGGAACTCTGGGGCTCTGACTT |
| | | CCGGCGTGCACACCTTCCCCGCCGTGCTGCAGAGCAGCGG |
| | | CCTGTACAGCCTGAGCAGCGTGGTGACAGTGCCCTCCAGC |
| | | TCTCTGGGAACCCAGACCTATATCTGCAACGTGAACCACAA |
| | | GCCCAGCAACACCAAGGTGGACAAGAGAGTGGAGCCCAAG |
| | | AGCTGCGACAAGACCCACACCTGCCCCCCCTGCCCAGCTC |
| | | CAGAACTGCTGGGAGGGCCTTCCGTGTTCCTGTTCCCCCC |
| | | CAAGCCCAAGGACACCCTGATGATCAGCAGGACCCCCGAG |
| | | GTGACCTGCGTGGTGGTGGACGTGTCCCACGAGGACCCAG |
| | | AGGTGAAGTTCAACTGGTACGTGGACGGCGTGGAGGTGCA |
| | | CAACGCCAAGACCAAGCCCAGAGAGGAGCAGTACAACAGC |
| | | ACCTACAGGGTGGTGTCCGTGCTGACCGTGCTGCACCAGG |
| | | ACTGGCTGAACGGCAAAGAATACAAGTGCAAAGTCTCCAAC |
| | | AAGGCCCTGCCAGCCCCAATCGAAAAGACAATCAGCAAGG |
| | | CCAAGGGCCAGCCACGGGAGCCCCAGGTGTACACCCTGCC |
| | | CCCCAGCCGGGAGGAGATGACCAAGAACCAGGTGTCCCTG |
| | | ACCTGTCTGGTGAAGGGCTTCTACCCCAGCGATATCGCCGT |
| | | GGAGTGGGAGAGCAACGGCCAGCCCGAGAACAACTACAAG |
| | | ACCACCCCCCCAGTGCTGGACAGCGACGGCAGCTTCTTCC |
| | | TGTACAGCAAGCTGACCGTGGACAAGTCCAGGTGGCAGCA |
| | | GGGCAACGTGTTCAGCTGCAGCGTGATGCACGAGGCCCTG |
| | | CACAACCACTACACCCAGAAGTCCCTGAGCCTGAGCCCCGG |
| | | CAAG |
| 170 | LCDR1 | SGDNIPQHSVH |
| | (Combined) | |
| 171 | LCDR2 | DDTERPS |
| | (Combined) | |
| 172 | LCDR3 | SSWDSSMDSVV |
| | (Combined) | |
| 173 | LCDR1 | SGDNIPQHSVH |
| | (Kabat) | |
| 174 | LCDR2 | DDTERPS |
| | (Kabat) | |
| 175 | LCDR3 | SSWDSSMDSVV |
| | (Kabat) | |
| 176 | LCDR1 | DNIPQHS |
| | (Chothia) | |
| 177 | LCDR2 | DDT |
| | (Chothia) | |
| 178 | LCDR3 | WDSSMDSV |
| | (Chothia) | |
| 179 | VL | SYELTQPLSVSVALGQTARITCSGDNIPQHSVHWYQQKPGQA |
| | | PVLVIYDDTERPSGIPERFSGSNSGNTATLTISRAQAGDEADYYCS |
| | | SWDSSMDSWFGGGTKLTVL |
| 180 | VH DNA | AGCTACGAGCTGACTCAGCCCCTGAGCGTCAGCGTGGCCC |
| | | TGGGTCAGACCGCTAGAATCACCTGTAGCGGCGATAATATC |
| | | CCTCAGCACTCAGTGCACTGGTATCAGCAGAAGCCCGGTC |
| | | AGGCCCCCGTGCTGGTGATCTACGACGACACCGAGCGGCC |
| | | TAGCGGAATCCCCGAGCGGTTTAGCGGCTCTAATAGCGGT |
| | | AACACCGCTACCCTGACTATCTCTAGGGCTCAGGCCGGCG |
| | | ACGAGGCCGACTACTACTGCTCTAGCTGGGATAGCTCTATG |
| | | GATAGCGTGGTGTTCGGCGGAGGCACTAAGCTGACCGTGCTG |
| 181 | Light Chain | SYELTQPLSVSVALGQTARITCSGDNIPQHSVHWYQQKPGQA |
| | | PVLVIYDDTERPSGIPERFSGSNSGNTATLTISRAQAGDEADYY |
| | | CSSWDSSMDSVVFGGGTKLTVLGQPKAAPSVTLFPPSSEELQ |
| | | ANKATLVCLISDFYPGAVTVAWKADSSPVKAGVETTTPSKQSN |
| | | NKYAASSYLSLTPEQWKSHRSYSCQVTHEGSTVEKTVAPTECS |
| 182 | Light Chain | AGCTACGAGCTGACTCAGCCCCTGAGCGTCAGCGTGGCCC |
| | DNA | TGGGTCAGACCGCTAGAATCACCTGTAGCGGCGATAATATC |
| | | CCTCAGCACTCAGTGCACTGGTATCAGCAGAAGCCCGGTC |
| | | AGGCCCCCGTGCTGGTGATCTACGACGACACCGAGCGGCC |
| | | TAGCGGAATCCCCGAGCGGTTTAGCGGCTCTAATAGCGGT |
| | | AACACCGCTACCCTGACTATCTCTAGGGCTCAGGCCGGCG |
| | | ACGAGGCCGACTACTACTGCTCTAGCTGGGATAGCTCTATG |
| | | GATAGCGTGGTGTTCGGCGGAGGCACTAAGCTGACCGTGC |
| | | TGGGTCAGCCTAAGGCTGCCCCCAGCGTGACCCTGTTCCC |
| | | CCCCAGCAGCGAGGAGCTGCAGGCCAACAAGGCCACCCTG |
| | | GTGTGCCTGATCAGCGACTTCTACCCAGGCGCCGTGACCG |
| | | TGGCCTGGAAGGCCGACAGCAGCCCCGTGAAGGCCGGCG |
| | | TGGAGACCACCACCCCCAGCAAGCAGAGCAACAACAAGTA |
| | | CGCCGCCAGCAGCTACCTGAGCCTGACCCCCGAGCAGTGG |
| | | AAGAGCCACAGGTCCTACAGCTGCCAGGTGACCCACGAGG |
| | | GCAGCACCGTGGAAAAGACCGTGGCCCCAACCGAGTGCAGC |

Antibody 008

| | | |
|---|---|---|
| 183 | HCDR1 | GGTFRDYAIS |
| | (Combined) | |
| 184 | HCDR2 | GIIPAFGTANYAQKFQG |
| | (Combined) | |
| 185 | HCDR3 | EQDPEYGYGGYPYEAMDV |
| | (Combined) | |
| 186 | HCDR1 | DYAIS |
| | (Kabat) | |
| 187 | HCDR2 | GIIPAFGTANYAQKFQG |
| | (Kabat) | |
| 188 | HCDR3 | EQDPEYGYGGYPYEAMDV |
| | (Kabat) | |
| 189 | HCDR1 | GGTFRDY |
| | (Chothia) | |
| 190 | HCDR2 | IPAFGT |
| | (Chothia) | |
| 191 | HCDR3 | EQDPEYGYGGYPYEAMDV |
| | (Chothia) | |
| 192 | VH | QVQLVQSGAEVKKPGSSVKVSCKASGGTFRDYAISWVRQAP |
| | | GQGLEWMGGIIPAFGTANYAQKFQGRVTITADESTSTAYMELS |
| | | SLRSEDTAVYYCAREQDPEYGYGGYPYEAMDVWGQGTLVTVSS |
| 193 | VH DNA | CAGGTGCAATTGGTGCAGAGCGGTGCCGAAGTGAAAAAAC |
| | | CGGGCAGCAGCGTGAAAGTTAGCTGCAAAGCATCCGGAGG |
| | | GACGTTTCGTGACTACGCTATCTCTTGGGTGCGCCAGGCCC |
| | | CGGGCCAGGGCCTCGAGTGGATGGGCGGTATCATCCCGG |
| | | CTTTCGGCACTGCGAACTACGCCCAGAAATTTCAGGGCCG |
| | | GGTGACCATTACCGCCGATGAAAGCACCAGCACCGCCTATA |
| | | TGGAACTGAGCAGCCTGCGCAGCGAAGATACGGCCGTGTA |
| | | TTATTGCGCGCGTGAACAGGACCCGGAATACGGTTACGGT |
| | | GGTTACCCGTATGAAGCTATGGATGTTTGGGGCCAAGGCACC |
| | | CTGGTGACTGTTAGCTCA |
| 194 | Heavy Chain | QVQLVQSGAEVKKPGSSVKVSCKASGGTFRDYAISWVRQAP |
| | | GQGLEWMGGIIPAFGTANYAQKFQGRVTITADESTSTAYMELS |
| | | SLRSEDTAVYYCAREQDPEYGYGGYPYEAMDVWGQGTLVTV |
| | | SSASTKGPSVFPLAPSSKSTSGGTAALGCLVKDYFPEPVTVS |
| | | WNSGALTSGVHTFPAVLQSSGLYSLSSVVTVPSSSLGTQTYIC |
| | | NVNHKPSNTKVDKRVEPKSCDKTHTCPPCPAPELLGGPSVFL |
| | | FPPKPKDTLMISRTPEVTCVVVDVSHEDPEVKFNWYVDGVEV |
| | | HNAKTKPREEQYASTYRVVSVLTVLHQDWLNGKEYKCKVSNK |
| | | ALPAPIEKTISKAKGQPREPQVYTLPPSREEMTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPGK |
| 195 | Heavy Chain | CAGGTGCAATTGGTGCAGAGCGGTGCCGAAGTGAAAAAAC |
| | DNA | CGGGCAGCAGCGTGAAAGTTAGCTGCAAAGCATCCGGAGG |
| | | GACGTTTCGTGACTACGCTATCTCTTGGGTGCGCCAGGCCC |
| | | CGGGCCAGGGCCTCGAGTGGATGGGCGGTATCATCCCGG |
| | | CTTTCGGCACTGCGAACTACGCCCAGAAATTTCAGGGCCG |
| | | GGTGACCATTACCGCCGATGAAAGCACCAGCACCGCCTATA |
| | | TGGAACTGAGCAGCCTGCGCAGCGAAGATACGGCCGTGTA |
| | | TTATTGCGCGCGTGAACAGGACCCGGAATACGGTTACGGT |
| | | GGTTACCCGTATGAAGCTATGGATGTTTGGGGCCAAGGCAC |
| | | CCTGGTGACTGTTAGCTCAGCCTCCACCAAGGGTCCATCG |
| | | GTCTTCCCCCTGGCACCCTCCTCCAAGAGCACCTCTGGGG |
| | | GCACAGCGGCCCTGGGCTGCCTGGTCAAGGACTACTTCCC |
| | | CGAACCGGTGACGGTGTCGTGGAACTCAGGCGCCCTGACC |
| | | AGCGGCGTGCACACCTTCCCGGCTGTCCTACAGTCCTCAG |
| | | GACTCTACTCCCTCAGCAGCGTGGTGACCGTGCCCTCCAG |
| | | CAGCTTGGGCACCCAGACCTACATCTGCAACGTGAATCACA |
| | | AGCCCAGCAACACCAAGGTGGACAAGAGAGTTGAGCCCAA |
| | | ATCTTGTGACAAAACTCACACATGCCCACCGTGCCCAGCAC |
| | | CTGAACTCCTGGGGGGACCGTCAGTCTTCCTCTTCCCCCCA |
| | | AAACCCAAGGACACCCTCATGATCTCCCGGACCCCTGAGGT |
| | | CACATGCGTGGTGGTGGACGTGAGCCACGAAGACCCTGAG |
| | | GTCAAGTTCAACTGGTACGTGGACGGCGTGGAGGTGCATA |
| | | ATGCCAAGACAAAGCCGCGGGAGGAGCAGTACGCCAGCAC |
| | | GTACCGGGTGGTCAGCGTCCTCACCGTCCTGCACCAGGAC |
| | | TGGCTGAATGGCAAGGAGTACAAGTGCAAGGTCTCCAACAA |
| | | AGCCCTCCCAGCCCCCATCGAGAAAACCATCTCCAAAGCCA |
| | | AAGGGCAGCCCCGAGAACCACAGGTGTACACCCTGCCCCC |
| | | ATCCCGGGAGGAGATGACCAAGAACCAGGTCAGCCTGACC |
| | | TGCCTGGTCAAAGGCTTCTATCCCAGCGACATCGCCGTGGA |
| | | GTGGGAGAGCAATGGGCAGCCGGAGAACAACTACAAGACC |
| | | ACGCCTCCCGTGCTGGACTCCGACGGCTCCTTCTTCCTCTA |
| | | CAGCAAGCTCACCGTGGACAAGAGCAGGTGGCAGCAGGG |
| | | GAACGTCTTCTCATGCTCCGTGATGCATGAGGCTCTGCACA |
| | | ACCACTACACGCAGAAGAGCCTCTCCCTGTCTCCGGGTAAA |
| 196 | LCDR1 | SGDNIPQHSVH |
| | (Combined) | |
| 197 | LCDR2 | DDTERPS |
| | (Combined) | |
| 198 | LCDR3 | SSWDSSMDSVV |
| | (Combined) | |
| 199 | LCDR1 | SGDNIPQHSVH |
| | (Kabat) | |
| 200 | LCDR2 | DDTERPS |
| | (Kabat) | |
| 201 | LCDR3 | SSWDSSMDSVV |
| | (Kabat) | |
| 202 | LCDR1 | DNIPQHS |
| | (Chothia) | |
| 203 | LCDR2 | DDT |
| | (Chothia) | |
| 204 | LCDR3 | WDSSMDSV |
| | (Chothia) | |
| 205 | VL | SYELTQPLSVSVALGQTARITCSGDNIPQHSVHWYQQKPGQA |
| | | PVLVIYDDTERPSGIPERFSGSNSGNTATLTISRAQAGDEADYYCS |
| | | SWDSSMPSWFGGGTKLTVL |
| 206 | VH DNA | AGCTACGAACTGACCCAGCCGCTGAGCGTGAGCGTGGCCC |
| | | TGGGCCAGACCGCGAGGATTACCTGTAGCGGCGATAACAT |
| | | CCCGCAGCATTCTGTTCATTGGTACCAGCAGAAACCGGGCC |
| | | AGGCGCCGGTGCTGGTGATCTACGACGACACTGAACGTCC |
| | | GAGCGGCATCCCGGAACGTTTTAGCGGATCCAACAGCGGC |
| | | AACACCGCGACCCTGACCATTAGCAGGGCCCAGGCGGGCG |
| | | ACGAAGCGGATTATTACTGCTCTTCTTGGGACTCTTCTATGG |
| | | ACTCTGTTGTGTTTGGCGGCGGCACGAAGTTAACCGTCCTA |
| 207 | Light Chain | SYELTQPLSVSVALGQTARITCSGDNIPQHSVHWYQQKPGQA |
| | | PVLVIYDDTERPSGIPERFSGSNSGNTATLTISRAQAGDEADYY |
| | | CSSWDSSMDSVVFGGGTKLTVLGQPKAAPSVTLFPPSSEELQ |
| | | ANKATLVCLISDFYPGAVTVAWKADSSPVKAGVETTTPSKQSN |
| | | NKYAASSYLSLTPEQWKSHRSYSCQVTHEGSTVEKTVAPTECS |
| 208 | Light Chain | AGCTACGAACTGACCCAGCCGCTGAGCGTGAGCGTGGCCC |
| | DNA | TGGGCCAGACCGCGAGGATTACCTGTAGCGGCGATAACAT |
| | | CCCGCAGCATTCTGTTCATTGGTACCAGCAGAAACCGGGCC |
| | | AGGCGCCGGTGCTGGTGATCTACGACGACACTGAACGTCC |
| | | GAGCGGCATCCCGGAACGTTTTAGCGGATCCAACAGCGGC |
| | | AACACCGCGACCCTGACCATTAGCAGGGCCCAGGCGGGCG |
| | | ACGAAGCGGATTATTACTGCTCTTCTTGGGACTCTTCTATGG |
| | | ACTCTGTTGTGTTTGGCGGCGGCACGAAGTTAACCGTCCTA |
| | | GGTCAGCCCAAGGCTGCCCCCTCGGTCACTCTGTTCCCGC |
| | | CCTCCTCTGAGGAGCTTCAAGCCAACAAGGCCACACTGGT |
| | | GTGTCTCATAAGTGACTTCTACCCGGGAGCCGTGACAGTGG |
| | | CCTGGAAGGCAGATAGCAGCCCCGTCAAGGCGGGAGTGGA |
| | | GACCACCACACCCTCCAAACAAAGCAACAACAAGTACGCGG |
| | | CCAGCAGCTATCTGAGCCTGACGCCTGAGCAGTGGAAGTC |
| | | CCACAGAAGCTACAGCTGCCAGGTCACGCATGAAGGGAGC |
| | | ACCGTGGAGAAGACAGTGGCCCCTACAGAATGTTCA |

Antibody 009

| | | |
|---|---|---|
| 209 | HCDR1 | GFTFPTHGLH |
| | (Combined) | |
| 210 | HCDR2 | AISYDASETNYADSVKG |
| | (Combined) | |
| 211 | HCDR3 | ESIGGYFDY |
| | (Combined) | |
| 212 | HCDR1 | THGLH |
| | (Kabat) | |
| 213 | HCDR2 | AISYDASETNYADSVKG |
| | (Kabat) | |
| 214 | HCDR3 | ESIGGYFDY |
| | (Kabat) | |
| 215 | HCDR1 | GFTFPTH |
| | (Chothia) | |
| 216 | HCDR2 | SYDASE |
| | (Chothia) | |
| 217 | HCDR3 | ESIGGYFDY |
| | (Chothia) | |
| 218 | VH | QVQLLESGGGLVQPGGSLRLSCAASGFTFPTHGLHVWRQAP |
| | | GKGLEVWSAISYDASETNYADSVKGRFTISRDNSKNTLYLQMN |
| | | SLRAEDTAVYYCARESIGGYFDYWGQGTLVTVSS |
| 219 | VH DNA | CAGGTGCAGCTGCTGGAATCAGGCGGCGGACTGGTGCAGC |
| | | CTGGCGGTAGCCTGAGACTGAGCTGCGCTGCTAGTGGCTT |
| | | CACCTTCCCTACTCACGGCCTGCACTGGGTCAGACAGGCC |
| | | CCTGGTAAAGGCCTGGAGTGGGTCAGCGCTATTAGCTACG |
| | | ACGCTAGTGAAACTAACTACGCCGATAGCGTGAAGGGCCG |
| | | GTTCACTATCTCTAGGGATAACTCTAAGAACACCCTGTACCT |
| | | GCAGATGAATAGCCTGAGAGCCGAGGACACCGCCGTCTAC |
| | | TACTGCGCTAGAGAGTCTATCGGCGGCTACTTCGACTACTG |
| | | GGGTCAGGGCACCCTGGTCACCGTGTCTAGC |
| 220 | Heavy Chain | QVQLLESGGGLVQPGGSLRLSCAASGFTFPTHGLHWVRQAP |
| | | GKGLEWVSAISYDASETNYADSVKGRFTISRDNSKNTLYLQMN |
| | | SLRAEDTAVYYCARESIGGYFDYWGQGTLVTVSSASTKGPSV |
| | | FPLAPSSKSTSGGTAALGCLVKDYFPEPVTVSWNSGALTSGV |
| | | HTFPAVLQSSGLYSLSSVVTVPSSSLGTQTYICNVNHKPSNTK |
| | | VDKRVEPKSCDKTHTCPPCPAPELLGGPSVFLFPPKPKDTLMI |
| | | SRTPEVTCVVVDVSHEDPEVKFNWYVDGVEVHNAKTKPREE |
| | | QYNSTYRVVSVLTVLHQDWLNGKEYKCKVSNKALPAPIEKTIS |
| | | KAKGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 221 | Heavy Chain | CAGGTGCAGCTGCTGGAATCAGGCGGCGGACTGGTGCAGC |
| | DNA | CTGGCGGTAGCCTGAGACTGAGCTGCGCTGCTAGTGGCTT |
| | | CACCTTCCCTACTCACGGCCTGCACTGGGTCAGACAGGCC |
| | | CCTGGTAAAGGCCTGGAGTGGGTCAGCGCTATTAGCTACG |
| | | ACGCTAGTGAAACTAACTACGCCGATAGCGTGAAGGGCCG |
| | | GTTCACTATCTCTAGGGATAACTCTAAGAACACCCTGTACCT |
| | | GCAGATGAATAGCCTGAGAGCCGAGGACACCGCCGTCTAC |
| | | TACTGCGCTAGAGAGTCTATCGGCGGCTACTTCGACTACTG |
| | | GGGTCAGGGCACCCTGGTCACCGTGTCTAGCGCTAGCACT |
| | | AAGGGCCCAAGTGTGTTTCCCCTGGCCCCCAGCAGCAAGT |
| | | CTACTTCCGGCGGAACTGCTGCCCTGGGTTGCCTGGTGAA |
| | | GGACTACTTCCCCGAGCCCGTGACAGTGTCCTGGAACTCT |
| | | GGGGCTCTGACTTCCGGCGTGCACACCTTCCCCGCCGTGC |
| | | TGCAGAGCAGCGGCCTGTACAGCCTGAGCAGCGTGGTGAC |
| | | AGTGCCCTCCAGCTCTCTGGGAACCCAGACCTATATCTGCA |
| | | ACGTGAACCACAAGCCCAGCAACACCAAGGTGGACAAGAG |
| | | AGTGGAGCCCAAGAGCTGCGACAAGACCCACACCTGCCCC |
| | | CCCTGCCCAGCTCCAGAACTGCTGGGAGGGCCTTCCGTGT |
| | | TCCTGTTCCCCCCCAAGCCCAAGGACACCCTGATGATCAGC |
| | | AGGACCCCCGAGGTGACCTGCGTGGTGGTGGACGTGTCCC |
| | | ACGAGGACCCAGAGGTGAAGTTCAACTGGTACGTGGACGG |
| | | CGTGGAGGTGCACAACGCCAAGACCAAGCCCAGAGAGGAG |
| | | CAGTACAACAGCACCTACAGGGTGGTGTCCGTGCTGACCG |
| | | TGCTGCACCAGGACTGGCTGAACGGCAAAGAATACAAGTG |
| | | CAAAGTCTCCAACAAGGCCCTGCCAGCCCCAATCGAAAAGA |
| | | CAATCAGCAAGGCCAAGGGCCAGCCACGGGAGCCCCAGGT |
| | | GTACACCCTGCCCCCCAGCCGGGAGGAGATGACCAAGAAC |
| | | CAGGTGTCCCTGACCTGTCTGGTGAAGGGCTTCTACCCCA |
| | | GCGATATCGCCGTGGAGTGGGAGAGCAACGGCCAGCCCG |
| | | AGAACAACTACAAGACCACCCCCCCAGTGCTGGACAGCGA |
| | | CGGCAGCTTCTTCCTGTACAGCAAGCTGACCGTGGACAAGT |
| | | CCAGGTGGCAGCAGGGCAACGTGTTCAGCTGCAGCGTGAT |
| | | GCACGAGGCCCTGCACAACCACTACACCCAGAAGTCCCTGAG |
| | | CCTGAGCCCCGGCAAG |
| 222 | LCDR1 | SGDALGKNTVS |
| | (Combined) | |
| 223 | LCDR2 | DDTDRPS |
| | (Combined) | |
| 224 | LCDR3 | SSTDLSTVV |
| | (Combined) | |
| 225 | LCDR1 | SGDALGKNTVS |
| | (Kabat) | |
| 226 | LCDR2 | DDTDRPS |
| | (Kabat) | |
| 227 | LCDR3 | SSTDLSTVV |
| | (Kabat) | |
| 228 | LCDR1 | DALGKNT |
| | (Chothia) | |
| 229 | LCDR2 | DDT |
| | (Chothia) | |
| 230 | LCDR3 | TDLSTV |
| | (Chothia) | |
| 231 | VL | SYELTQPLSVSVALGQTARITCSGDALGKNTVSWYQQKPGQA |
| | | PVLVIYDDTDRPSGIPERFSGSNSGNTATLTISRAQAGDEADYYC |
| | | SSTDLSTVVFGGGTKLTVL |
| 232 | VH DNA | AGCTACGAGCTGACTCAGCCCCTGAGCGTCAGCGTGGCCC |
| | | TGGGTCAGACCGCTAGAATCACCTGTAGCGGCGACGCCCT |
| | | GGGTAAAAACACCGTCAGCTGGTATCAGCAGAAGCCCGGT |
| | | CAGGCCCCCGTGCTGGTGATCTACGACGACACCGATAGAC |
| | | CTAGCGGAATCCCCGAGCGGTTTAGCGGCTCTAATAGCGG |
| | | TAACACCGCTACCCTGACTATCTCTAGGGCTCAGGCCGGC |
| | | GACGAGGCCGACTACTACTGCTCTAGCACCGACCTGAGCA |
| | | CCGTGGTGTTCGGCGGAGGCACTAAGCTGACCGTGCTG |
| 233 | Light Chain | SYELTQPLSVSVALGQTARITCSGDALGKNTVSWYQQKPGQA |
| | | PVLVIYDDTDRPSGIPERFSGSNSGNTATLTISRAQAGDEADYY |
| | | CSSTDLSTVVFGGGTKLTVLGQPKAAPSVTLFPPSSEELQANK |
| | | ATLVCLISDFYPGAVTVAWKADSSPVKAGVETTTPSKQSNNKY |
| | | AASSYLSLTPEQWKSHRSYSCQVTHEGSTVEKTVAPTECS |
| 234 | Light Chain | AGCTACGAGCTGACTCAGCCCCTGAGCGTCAGCGTGGCCC |
| | DNA | TGGGTCAGACCGCTAGAATCACCTGTAGCGGCGACGCCCT |
| | | GGGTAAAAACACCGTCAGCTGGTATCAGCAGAAGCCCGGT |
| | | CAGGCCCCCGTGCTGGTGATCTACGACGACACCGATAGAC |
| | | CTAGCGGAATCCCCGAGCGGTTTAGCGGCTCTAATAGCGG |
| | | TAACACCGCTACCCTGACTATCTCTAGGGCTCAGGCCGGC |
| | | GACGAGGCCGACTACTACTGCTCTAGCACCGACCTGAGCA |
| | | CCGTGGTGTTCGGCGGAGGCACTAAGCTGACCGTGCTGGG |
| | | TCAGCCTAAGGCTGCCCCCAGCGTGACCCTGTTCCCCCCC |
| | | AGCAGCGAGGAGCTGCAGGCCAACAAGGCCACCCTGGTGT |
| | | GCCTGATCAGCGACTTCTACCCAGGCGCCGTGACCGTGGC |
| | | CTGGAAGGCCGACAGCAGCCCCGTGAAGGCCGGCGTGGA |
| | | GACCACCACCCCCAGCAAGCAGAGCAACAACAAGTACGCC |
| | | GCCAGCAGCTACCTGAGCCTGACCCCCGAGCAGTGGAAGA |
| | | GCCACAGGTCCTACAGCTGCCAGGTGACCCACGAGGGCAG |
| | | CACCGTGGAAAAGACCGTGGCCCCAACCGAGTGCAGC |

Antibody 010

| | | |
|---|---|---|
| 235 | HCDR1 | GFTFPTHGLH |
| | (Combined) | |
| 236 | HCDR2 | AISYDASETNYADSVKG |
| | (Combined) | |
| 237 | HCDR3 | ESIGGYFDY |
| | (Combined) | |
| 238 | HCDR1 | THGLH |
| | (Kabat) | |
| 239 | HCDR2 | AISYDASETNYADSVKG |
| | (Kabat) | |
| 240 | HCDR3 | ESIGGYFDY |
| | (Kabat) | |
| 241 | HCDR1 | GFTFPTH |
| | (Chothia) | |
| 242 | HCDR2 | SYDASE |
| | (Chothia) | |
| 243 | HCDR3 | ESIGGYFDY |
| | (Chothia) | |
| 244 | VH | QVQLLESGGGLVQPGGSLRLSCAASGFTFPTHGLHVWRQAP |
| | | GKGLEWVSAISYDASETNYADSVKGRFTISRDNSKNTLYLQMN |
| | | SLRAEDTAVYYCARESIGGYFDYWGQGTLVTVSS |
| 245 | VH DNA | CAGGTGCAATTGCTGGAAAGCGGCGGTGGCCTGGTGCAGC |
| | | CGGGTGGCAGCCTGCGTCTGAGCTGCGCGGCGTCCGGATT |
| | | CACCTTTCCTACTCATGGTCTGCATTGGGTGCGCCAGGCCC |
| | | CGGGCAAAGGTCTCGAGTGGGTTTCCGCTATCTCTTACGAC |
| | | GCCTCTGAAACCAACTATGCGGATAGCGTGAAAGGCCGCTT |
| | | TACCATCAGCCGCGATAATTCGAAAAACACCCTGTATCTGC |
| | | AAATGAACAGCCTGCGTGCGGAAGATACGGCCGTGTATTAT |
| | | TGCGCGCGTGAATCTATCGGTGGTTACTTCGATTACTGGGG |
| | | CCAAGGCACCCTGGTGACTGTTAGCTCA |
| 246 | Heavy Chain | QVQLLESGGGLVQPGGSLRLSCAASGFTFPTHGLHVWRQAP |
| | | GKGLEWVSAISYDASETNYADSVKGRFTISRDNSKNTLYLQMN |
| | | SLRAEDTAVYYCARESIGGYFDYWGQGTLVTVSSASTKGPSV |
| | | FPLAPSSKSTSGGTAALGCLVKDYFPEPVTVSWNSGALTSGV |
| | | HTFPAVLQSSGLYSLSSVVTVPSSSLGTQTYICNVNHKPSNTK |
| | | VDKRVEPKSCDKTHTCPPCPAPELLGGPSVFLFPPKPKDTLMI |
| | | SRTPEVTCVVVDVSHEDPEVKFNWYVDGVEVHNAKTKPREE |
| | | QYASTYRVVSVLTVLHQDWLNGKEYKCKVSNKALPAPIEKTIS |
| | | KAKGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 247 | Heavy Chain | CAGGTGCAATTGCTGGAAAGCGGCGGTGGCCTGGTGCAGC |
| | DNA | CGGGTGGCAGCCTGCGTCTGAGCTGCGCGGCGTCCGGATT |
| | | CACCTTTCCTACTCATGGTCTGCATTGGGTGCGCCAGGCCC |
| | | CGGGCAAAGGTCTCGAGTGGGTTTCCGCTATCTCTTACGAC |
| | | GCCTCTGAAACCAACTATGCGGATAGCGTGAAAGGCCGCTT |
| | | TACCATCAGCCGCGATAATTCGAAAAACACCCTGTATCTGC |
| | | AAATGAACAGCCTGCGTGCGGAAGATACGGCCGTGTATTAT |
| | | TGCGCGCGTGAATCTATCGGTGGTTACTTCGATTACTGGGG |
| | | CCAAGGCACCCTGGTGACTGTTAGCTCAGCCTCCACCAAG |
| | | GGTCCATCGGTCTTCCCCCTGGCACCCTCCTCCAAGAGCA |
| | | CCTCTGGGGGCACAGCGGCCCTGGGCTGCCTGGTCAAGG |
| | | ACTACTTCCCCGAACCGGTGACGGTGTCGTGGAACTCAGG |
| | | CGCCCTGACCAGCGGCGTGCACACCTTCCCGGCTGTCCTA |
| | | CAGTCCTCAGGACTCTACTCCCTCAGCAGCGTGGTGACCGT |
| | | GCCCTCCAGCAGCTTGGGCACCCAGACCTACATCTGCAAC |
| | | GTGAATCACAAGCCCAGCAACACCAAGGTGGACAAGAGAG |
| | | TTGAGCCCAAATCTTGTGACAAAACTCACACATGCCCACCG |
| | | TGCCCAGCACCTGAACTCCTGGGGGGACCGTCAGTCTTCC |
| | | TCTTCCCCCCAAAACCCAAGGACACCCTCATGATCTCCCGG |
| | | ACCCCTGAGGTCACATGCGTGGTGGTGGACGTGAGCCACG |
| | | AAGACCCTGAGGTCAAGTTCAACTGGTACGTGGACGGCGT |
| | | GGAGGTGCATAATGCCAAGACAAAGCCGCGGGAGGAGCAG |
| | | TACGCCAGCACGTACCGGGTGGTCAGCGTCCTCACCGTCC |
| | | TGCACCAGGACTGGCTGAATGGCAAGGAGTACAAGTGCAA |
| | | GGTCTCCAACAAAGCCCTCCCAGCCCCCATCGAGAAAACCA |
| | | TCTCCAAAGCCAAAGGGCAGCCCCGAGAACCACAGGTGTA |
| | | CACCCTGCCCCCATCCCGGGAGGAGATGACCAAGAACCAG |
| | | GTCAGCCTGACCTGCCTGGTCAAAGGCTTCTATCCCAGCGA |
| | | CATCGCCGTGGAGTGGGAGAGCAATGGGCAGCCGGAGAA |
| | | CAACTACAAGACCACGCCTCCCGTGCTGGACTCCGACGGC |
| | | TCCTTCTTCCTCTACAGCAAGCTCACCGTGGACAAGAGCAG |
| | | GTGGCAGCAGGGGAACGTCTTCTCATGCTCCGTGATGCAT |
| | | GAGGCTCTGCACAACCACTACACGCAGAAGAGCCTCTCCCT |
| | | GTCTCCGGGTAAA |
| 248 | LCDR1 | SGDALGKNTVS |
| | (Combined) | |
| 249 | LCDR2 | DDTDRPS |
| | (Combined) | |
| 250 | LCDR3 | SSTDLSTVV |
| | (Combined) | |
| 251 | LCDR1 | SGDALGKNTVS |
| | (Kabat) | |
| 252 | LCDR2 | DDTDRPS |
| | (Kabat) | |
| 253 | LCDR3 | SSTDLSTVV |
| | (Kabat) | |
| 254 | LCDR1 | DALGKNT |
| | (Chothia) | |
| 255 | LCDR2 | DDT |
| | (Chothia) | |
| 256 | LCDR3 | TDLSTV |
| | (Chothia) | |
| 257 | VL | SYELTQPLSVSVALGQTARITCSGDALGKNTVSWYQQKPGQA |
| | | PVLVIYQDTDRPSGIPERFSGSNSGNTATLTISRAQAGDEADYY |
| | | CSSTDLSTVVFGGGTKLTVL |
| 258 | VH DNA | AGCTACGAACTGACCCAGCCGCTGAGCGTGAGCGTGGCCC |
| | | TGGGCCAGACCGCGAGGATTACCTGTAGCGGCGATGCTCT |
| | | GGGTAAAAACACTGTTTCTTGGTACCAGCAGAAACCGGGCC |
| | | AGGCGCCGGTGCTGGTGATCTACGACGACACTGACCGTCC |
| | | GAGCGGCATCCCGGAACGTTTTAGCGGATCCAACAGCGGC |
| | | AACACCGCGACCCTGACCATTAGCAGGGCCCAGGCGGGCG |
| | | ACGAAGCGGATTATTACTGCTCTTCTACTGACCTGTCTACTG |
| | | TTGTGTTTGGCGGCGGCACGAAGTTAACCGTCCTA |
| 259 | Light Chain | SYELTQPLSVSVALGQTARITCSGDALGKNTVSWYQQKPGQA |
| | | PVLVIYDDTDRPSGIPERFSGSNSGNTATLTISRAQAGDEADYY |
| | | CSSTDLSTWFGGGTKLTVLGQPKAAPSVTLFPPSSEELQANK |
| | | ATLVCLISDFYPGAVTVAWKADSSPVKAGVETTTPSKQSNNKY |
| | | AASSYLSLTPEQWKSHRSYSCQVTHEGSTVEKTVAPTECS |
| 260 | Light Chain | AGCTACGAACTGACCCAGCCGCTGAGCGTGAGCGTGGCCC |
| | DNA | TGGGCCAGACCGCGAGGATTACCTGTAGCGGCGATGCTCT |
| | | GGGTAAAAACACTGTTTCTTGGTACCAGCAGAAACCGGGCC |
| | | AGGCGCCGGTGCTGGTGATCTACGACGACACTGACCGTCC |
| | | GAGCGGCATCCCGGAACGTTTTAGCGGATCCAACAGCGGC |
| | | AACACCGCGACCCTGACCATTAGCAGGGCCCAGGCGGGCG |
| | | ACGAAGCGGATTATTACTGCTCTTCTACTGACCTGTCTACTG |
| | | TTGTGTTTGGCGGCGGCACGAAGTTAACCGTCCTAGGTCAG |
| | | CCCAAGGCTGCCCCCTCGGTCACTCTGTTCCCGCCCTCCT |
| | | CTGAGGAGCTTCAAGCCAACAAGGCCACACTGGTGTGTCTC |
| | | ATAAGTGACTTCTACCCGGGAGCCGTGACAGTGGCCTGGA |
| | | AGGCAGATAGCAGCCCCGTCAAGGCGGGAGTGGAGACCAC |
| | | CACACCCTCCAAACAAAGCAACAACAAGTACGCGGCCAGCA |
| | | GCTATCTGAGCCTGACGCCTGAGCAGTGGAAGTCCCACAG |
| | | AAGCTACAGCTGCCAGGTCACGCATGAAGGGAGCACCGTG |
| | | GAGAAGACAGTGGCCCCTACAGAATGTTCA |

Antibody 011

| | | |
|---|---|---|
| 261 | HCDR1 | GFTFPTHGLH |
| | (Combined) | |
| 262 | HCDR2 | AISYEGSETNYADSVKG |
| | (Combined) | |
| 263 | HCDR3 | ESIGGYFDY |
| | (Combined) | |
| 264 | HCDR1 | THGLH |
| | (Kabat) | |
| 265 | HCDR2 | AISYEGSETNYADSVKG |
| | (Kabat) | |
| 266 | HCDR3 | ESIGGYFDY |
| | (Kabat) | |
| 267 | HCDR1 | GFTFPTH |
| | (Chothia) | |
| 268 | HCDR2 | SYEGSE |
| | (Chothia) | |
| 269 | HCDR3 | ESIGGYFDY |
| | (Chothia) | |
| 270 | VH | QVQLLESGGGLVQPGGSLRLSCAASGFTFPTHGLHVWRQAP |
| | | GKGLEWVSAISYEGSETNYADSVKGRFTISRDNSKNTLYLQMN |
| | | SLRAEDTAVYYCARESIGGYFDYWGQGTLVTVSS |
| 271 | VH DNA | CAGGTGCAGCTGCTGGAATCAGGCGGCGGACTGGTGCAGC |
| | | CTGGCGGTAGCCTGAGACTGAGCTGCGCTGCTAGTGGCTT |
| | | CACCTTCCCTACTCACGGCCTGCACTGGGTCAGACAGGCC |
| | | CCTGGTAAAGGCCTGGAGTGGGTCAGCGCTATTAGCTACG |
| | | AGGGTAGCGAGACTAACTACGCCGATAGCGTGAAGGGCCG |
| | | GTTCACTATCTCTAGGGATAACTCTAAGAACACCCTGTACCT |
| | | GCAGATGAATAGCCTGAGAGCCGAGGACACCGCCGTCTAC |
| | | TACTGCGCTAGAGAGTCTATCGGCGGCTACTTCGACTACTG |
| | | GGGTCAGGGCACCCTGGTCACCGTGTCTAGC |
| 272 | Heavy Chain | QVGLLESGGGLVQPGGSLRLSCAASGFTFPTHGLHVWRQAP |
| | | GKGLEVWSAISYEGSETNYADSVKGRFTISRDNSKNTLYLQMN |
| | | SLRAEDTAVYYCARESIGGYFDYWGQGTLVTVSSASTKGPSV |
| | | FPLAPSSKSTSGGTAALGCLVKDYFPEPVTVSWNSGALTSGV |
| | | HTFPAVLQSSGLYSLSSVVTVPSSSLGTQTYICNVNHKPSNTK |
| | | VDKRVEPKSCDKTHTCPPCPAPELLGGPSVFLFPPKPKDTLMI |
| | | SRTPEVTCVVVDVSHEDPEVKFNWYVDGVEVHNAKTKPREE |
| | | QYNSTYRVVSVLTVLHQDWLNGKEYKCKVSNKALPAPIEKTIS |
| | | KAKGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 273 | Heavy Chain | CAGGTGCAGCTGCTGGAATCAGGCGGCGGACTGGTGCAGC |
| | DNA | CTGGCGGTAGCCTGAGACTGAGCTGCGCTGCTAGTGGCTT |
| | | CACCTTCCCTACTCACGGCCTGCACTGGGTCAGACAGGCC |
| | | CCTGGTAAAGGCCTGGAGTGGGTCAGCGCTATTAGCTACG |
| | | AGGGTAGCGAGACTAACTACGCCGATAGCGTGAAGGGCCG |
| | | GTTCACTATCTCTAGGGATAACTCTAAGAACACCCTGTACCT |
| | | GCAGATGAATAGCCTGAGAGCCGAGGACACCGCCGTCTAC |
| | | TACTGCGCTAGAGAGTCTATCGGCGGCTACTTCGACTACTG |
| | | GGGTCAGGGCACCCTGGTCACCGTGTCTAGCGCTAGCACT |
| | | AAGGGCCCAAGTGTGTTTCCCCTGGCCCCCAGCAGCAAGT |
| | | CTACTTCCGGCGGAACTGCTGCCCTGGGTTGCCTGGTGAA |
| | | GGACTACTTCCCCGAGCCCGTGACAGTGTCCTGGAACTCT |
| | | GGGGCTCTGACTTCCGGCGTGCACACCTTCCCCGCCGTGC |
| | | TGCAGAGCAGCGGCCTGTACAGCCTGAGCAGCGTGGTGAC |
| | | AGTGCCCTCCAGCTCTCTGGGAACCCAGACCTATATCTGCA |
| | | ACGTGAACCACAAGCCCAGCAACACCAAGGTGGACAAGAG |
| | | AGTGGAGCCCAAGAGCTGCGACAAGACCCACACCTGCCCC |
| | | CCCTGCCCAGCTCCAGAACTGCTGGGAGGGCCTTCCGTGT |
| | | TCCTGTTCCCCCCCAAGCCCAAGGACACCCTGATGATCAGC |
| | | AGGACCCCCGAGGTGACCTGCGTGGTGGTGGACGTGTCCC |
| | | ACGAGGACCCAGAGGTGAAGTTCAACTGGTACGTGGACGG |
| | | CGTGGAGGTGCACAACGCCAAGACCAAGCCCAGAGAGGAG |
| | | CAGTACAACAGCACCTACAGGGTGGTGTCCGTGCTGACCG |
| | | TGCTGCACCAGGACTGGCTGAACGGCAAAGAATACAAGTG |
| | | CAAAGTCTCCAACAAGGCCCTGCCAGCCCCAATCGAAAAGA |
| | | CAATCAGCAAGGCCAAGGGCCAGCCACGGGAGCCCCAGGT |
| | | GTACACCCTGCCCCCCAGCCGGGAGGAGATGACCAAGAAC |
| | | CAGGTGTCCCTGACCTGTCTGGTGAAGGGCTTCTACCCCA |
| | | GCGATATCGCCGTGGAGTGGGAGAGCAACGGCCAGCCCG |
| | | AGAACAACTACAAGACCACCCCCCCAGTGCTGGACAGCGA |
| | | CGGCAGCTTCTTCCTGTACAGCAAGCTGACCGTGGACAAGT |
| | | CCAGGTGGCAGCAGGGCAACGTGTTCAGCTGCAGCGTGAT |
| | | GCACGAGGCCCTGCACAACCACTACACCCAGAAGTCCCTGA |
| | | GCCTGAGCCCCGGCAAG |
| 274 | LCDR1 | SGDALGKNTVS |
| | (Combined) | |
| 275 | LCDR2 | DDTDRPS |
| | (Combined) | |
| 276 | LCDR3 | SSTDLSTVV |
| | (Combined) | |
| 277 | LCDR1 | SGDALGKNTVS |
| | (Kabat) | |
| 278 | LCDR2 | DDTDRPS |
| | (Kabat) | |
| 279 | LCDR3 | SSTDLSTVV |
| | (Kabat) | |
| 280 | LCDR1 | DALGKNT |
| | (Chothia) | |
| 281 | LCDR2 | DDT |
| | (Chothia) | |
| 282 | LCDR3 | TDLSTV |
| | (Chothia) | |
| 283 | VL | SYELTQPLSVSVALGQTARITCSGDALGKNTVSWYQQKPGQA |
| | | PVLVIYDDTDRPSGIPERFSGSNSGNTATLTISRAQAGDEADYY |
| | | CSSTDLSTWFGGGTKLTVL |
| 284 | VH DNA | AGCTACGAGCTGACTCAGCCCCTGAGCGTCAGCGTGGCCC |
| | | TGGGTCAGACCGCTAGAATCACCTGTAGCGGCGACGCCCT |
| | | GGGTAAAAACACCGTCAGCTGGTATCAGCAGAAGCCCGGT |
| | | CAGGCCCCCGTGCTGGTGATCTACGACGACACCGATAGAC |
| | | CTAGCGGAATCCCCGAGCGGTTTAGCGGCTCTAATAGCGG |
| | | TAACACCGCTACCCTGACTATCTCTAGGGCTCAGGCCGGC |
| | | GACGAGGCCGACTACTACTGCTCTAGCACCGACCTGAGCA |
| | | CCGTGGTGTTCGGCGGAGGCACTAAGCTGACCGTGCTG |
| 285 | Light Chain | SYELTQPLSVSVALGQTARITCSGDALGKNTVSWYQQKPGQA |
| | | PVLVIYDDTDRPSGIPERFSGSNSGNTATLTISRAQAGDEADYY |
| | | CSSTDLSTVVFGGGTKLTVLGQPKAAPSVTLFPPSSEELQANK |
| | | ATLVCLISDFYPGAVTVAWKADSSPVKAGVETTTPSKQSNNKY |
| | | AASSYLSLTPEQWKSHRSYSCQVTHEGSTVEKTVAPTECS |
| 286 | Light Chain | AGCTACGAGCTGACTCAGCCCCTGAGCGTCAGCGTGGCCC |
| | DNA | TGGGTCAGACCGCTAGAATCACCTGTAGCGGCGACGCCCT |
| | | GGGTAAAAACACCGTCAGCTGGTATCAGCAGAAGCCCGGT |
| | | CAGGCCCCCGTGCTGGTGATCTACGACGACACCGATAGAC |
| | | CTAGCGGAATCCCCGAGCGGTTTAGCGGCTCTAATAGCGG |
| | | TAACACCGCTACCCTGACTATCTCTAGGGCTCAGGCCGGC |
| | | GACGAGGCCGACTACTACTGCTCTAGCACCGACCTGAGCA |
| | | CCGTGGTGTTCGGCGGAGGCACTAAGCTGACCGTGCTGGG |
| | | TCAGCCTAAGGCTGCCCCCAGCGTGACCCTGTTCCCCCCC |
| | | AGCAGCGAGGAGCTGCAGGCCAACAAGGCCACCCTGGTGT |
| | | GCCTGATCAGCGACTTCTACCCAGGCGCCGTGACCGTGGC |
| | | CTGGAAGGCCGACAGCAGCCCCGTGAAGGCCGGCGTGGA |
| | | GACCACCACCCCCAGCAAGCAGAGCAACAACAAGTACGCC |
| | | GCCAGCAGCTACCTGAGCCTGACCCCCGAGCAGTGGAAGA |
| | | GCCACAGGTCCTACAGCTGCCAGGTGACCCACGAGGGCAG |
| | | CACCGTGGAAAAGACCGTGGCCCCAACCGAGTGCAGC |

Antibody 012

| | | |
|---|---|---|
| 287 | HCDR1 | GFTFPTHGLH |
| | (Combined) | |
| 288 | HCDR2 | AISYEGSETNYADSVKG |
| | (Combined) | |
| 289 | HCDR3 | ESIGGYFDY |
| | (Combined) | |
| 290 | HCDR1 | THGLH |
| | (Kabat) | |
| 291 | HCDR2 | AISYEGSETNYADSVKG |
| | (Kabat) | |
| 292 | HCDR3 | ESIGGYFDY |
| | (Kabat) | |
| 293 | HCDR1 | GFTFPTH |
| | (Chothia) | |
| 294 | HCDR2 | SYEGSE |
| | (Chothia) | |
| 295 | HCDR3 | ESIGGYFDY |
| | (Chothia) | |
| 296 | VH | QVQLLESGGGLVQPGGSLRLSCAASGFTFPTHGLHVWRQAP |
| | | GKGLEWVSAISYEGSETNYADSVKGRFTISRDNSKNTLYLQMN |
| | | SLRAEDTAVYYCARESIGGYFDYWGQGTLVTVSS |
| 297 | VH DNA | CAGGTGCAATTGCTGGAAAGCGGCGGTGGCCTGGTGCAGC |
| | | CGGGTGGCAGCCTGCGTCTGAGCTGCGCGGCGTCCGGATT |
| | | CACCTTTCCTACTCATGGTCTGCATTGGGTGCGCCAGGCCC |
| | | CGGGCAAAGGTCTCGAGTGGGTTTCCGCTATCTCTTACGAG |
| | | GGTTCTGAAACCAACTATGCGGATAGCGTGAAAGGCCGCTT |
| | | TACCATCAGCCGCGATAATTCGAAAAACACCCTGTATCTGC |
| | | AAATGAACAGCCTGCGTGCGGAAGATACGGCCGTGTATTAT |
| | | TGCGCGCGTGAATCTATCGGTGGTTACTTCGATTACTGGGG |
| | | CCAAGGCACCCTGGTGACTGTTAGCTCA |
| 298 | Heavy Chain | QVQLLESGGGLVQPGGSLRLSCAASGFTFPTHGLHVWRQAP |
| | | GKGLEWVSAISYEGSETNYADSVKGRFTISRDNSKNTLYLQMN |
| | | SLRAEDTAVYYCARESIGGYFDYWGQGTLVTVSSASTKGPSV |
| | | FPLAPSSKSTSGGTAALGCLVKDYFPEPVTVSWNSGALTSGV |
| | | HTFPAVLQSSGLYSLSSVVTVPSSSLGTQTYICNVNHKPSNTK |
| | | VDKRVEPKSCDKTHTCPPCPAPELLGGPSVFLFPPKPKDTLMI |
| | | SRTPEVTCVVVDVSHEDPEVKFNWYVDGVEVHNAKTKPREE |
| | | QYASTYRVVSVLTVLHQDWLNGKEYKCKVSNKALPAPIEKTIS |
| | | KAKGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 299 | Heavy Chain | CAGGTGCAATTGCTGGAAAGCGGCGGTGGCCTGGTGCAGC |
| | DNA | CGGGTGGCAGCCTGCGTCTGAGCTGCGCGGCGTCCGGATT |
| | | CACCTTTCCTACTCATGGTCTGCATTGGGTGCGCCAGGCCC |
| | | CGGGCAAAGGTCTCGAGTGGGTTTCCGCTATCTCTTACGAG |
| | | GGTTCTGAAACCAACTATGCGGATAGCGTGAAAGGCCGCTT |
| | | TACCATCAGCCGCGATAATTCGAAAAACACCCTGTATCTGC |
| | | AAATGAACAGCCTGCGTGCGGAAGATACGGCCGTGTATTAT |
| | | TGCGCGCGTGAATCTATCGGTGGTTACTTCGATTACTGGGG |
| | | CCAAGGCACCCTGGTGACTGTTAGCTCAGCCTCCACCAAG |
| | | GGTCCATCGGTCTTCCCCCTGGCACCCTCCTCCAAGAGCA |
| | | CCTCTGGGGGCACAGCGGCCCTGGGCTGCCTGGTCAAGG |
| | | ACTACTTCCCCGAACCGGTGACGGTGTCGTGGAACTCAGG |
| | | CGCCCTGACCAGCGGCGTGCACACCTTCCCGGCTGTCCTA |
| | | CAGTCCTCAGGACTCTACTCCCTCAGCAGCGTGGTGACCGT |
| | | GCCCTCCAGCAGCTTGGGCACCCAGACCTACATCTGCAAC |
| | | GTGAATCACAAGCCCAGCAACACCAAGGTGGACAAGAGAG |
| | | TTGAGCCCAAATCTTGTGACAAAACTCACACATGCCCACCG |
| | | TGCCCAGCACCTGAACTCCTGGGGGGACCGTCAGTCTTCC |
| | | TCTTCCCCCCAAAACCCAAGGACACCCTCATGATCTCCCGG |
| | | ACCCCTGAGGTCACATGCGTGGTGGTGGACGTGAGCCACG |
| | | AAGACCCTGAGGTCAAGTTCAACTGGTACGTGGACGGCGT |
| | | GGAGGTGCATAATGCCAAGACAAAGCCGCGGGAGGAGCAG |
| | | TACGCCAGCACGTACCGGGTGGTCAGCGTCCTCACCGTCC |
| | | TGCACCAGGACTGGCTGAATGGCAAGGAGTACAAGTGCAA |
| | | GGTCTCCAACAAAGCCCTCCCAGCCCCCATCGAGAAAACCA |
| | | TCTCCAAAGCCAAAGGGCAGCCCCGAGAACCACAGGTGTA |
| | | CACCCTGCCCCCATCCCGGGAGGAGATGACCAAGAACCAG |
| | | GTCAGCCTGACCTGCCTGGTCAAAGGCTTCTATCCCAGCGA |
| | | CATCGCCGTGGAGTGGGAGAGCAATGGGCAGCCGGAGAA |
| | | CAACTACAAGACCACGCCTCCCGTGCTGGACTCCGACGGC |
| | | TCCTTCTTCCTCTACAGCAAGCTCACCGTGGACAAGAGCAG |
| | | GTGGCAGCAGGGGAACGTCTTCTCATGCTCCGTGATGCAT |
| | | GAGGCTCTGCACAACCACTACACGCAGAAGAGCCTCTCCCT |
| | | GTCTCCGGGTAAA |
| 300 | LCDR1 | SGDALGKNTVS |
| | (Combined) | |
| 301 | LCDR2 | DDTDRPS |
| | (Combined) | |
| 302 | LCDR3 | SSTDLSTVV |
| | (Combined) | |
| 303 | LCDR1 | SGDALGKNTVS |
| | (Kabat) | |
| 304 | LCDR2 | DDTDRPS |
| | (Kabat) | |
| 305 | LCDR3 | SSTDLSTVV |
| | (Kabat) | |
| 306 | LCDR1 | DALGKNT |
| | (Chothia) | |
| 307 | LCDR2 | DDT |
| | (Chothia) | |
| 308 | LCDR3 | TDLSTV |
| | (Chothia) | |
| 309 | VL | SYELTQPLSVSVALGQTARITCSGDALGKNTVSWYQQKPGQA |
| | | PVLVIYDDTDRPSGIPERFSGSNSGNTATLTISRAQAGDEADYY |
| | | CSSTDLSTVVFGGGTKLTVL |
| 310 | VH DNA | AGCTACGAACTGACCCAGCCGCTGAGCGTGAGCGTGGCCC |
| | | TGGGCCAGACCGCGAGGATTACCTGTAGCGGCGATGCTCT |
| | | GGGTAAAAACACTGTTTCTTGGTACCAGCAGAAACCGGGCC |
| | | AGGCGCCGGTGCTGGTGATCTACGACGACACTGACCGTCC |
| | | GAGCGGCATCCCGGAACGTTTTAGCGGATCCAACAGCGGC |
| | | AACACCGCGACCCTGACCATTAGCAGGGCCCAGGCGGGCG |
| | | ACGAAGCGGATTATTACTGCTCTTCTACTGACCTGTCTACTG |
| | | TTGTGTTTGGCGGCGGCACGAAGTTAACCGTCCTA |
| 311 | Light Chain | SYELTQPLSVSVALGQTARITCSGDALGKNTVSWYQQKPGQA |
| | | PVLVIYDDTDRPSGIPERFSGSNSGNTATLTISRAQAGDEADYY |
| | | CSSTDLSTVVFGGGTKLTVLGQPKAAPSVTLFPPSSEELQANK |
| | | ATLVCLISDFYPGAVTVAWKADSSPVKAGVETTTPSKQSNNKY |
| | | AASSYLSLTPEQWKSHRSYSCQVTHEGSTVEKTVAPTECS |
| 312 | Light Chain | AGCTACGAACTGACCCAGCCGCTGAGCGTGAGCGTGGCCC |
| | DNA | TGGGCCAGACCGCGAGGATTACCTGTAGCGGCGATGCTCT |
| | | GGGTAAAAACACTGTTTCTTGGTACCAGCAGAAACCGGGCC |
| | | AGGCGCCGGTGCTGGTGATCTACGACGACACTGACCGTCC |
| | | GAGCGGCATCCCGGAACGTTTTAGCGGATCCAACAGCGGC |
| | | AACACCGCGACCCTGACCATTAGCAGGGCCCAGGCGGGCG |
| | | ACGAAGCGGATTATTACTGCTCTTCTACTGACCTGTCTACTG |
| | | TTGTGTTTGGCGGCGGCACGAAGTTAACCGTCCTAGGTCAG |
| | | CCCAAGGCTGCCCCCTCGGTCACTCTGTTCCCGCCCTCCT |
| | | CTGAGGAGCTTCAAGCCAACAAGGCCACACTGGTGTGTCTC |
| | | ATAAGTGACTTCTACCCGGGAGCCGTGACAGTGGCCTGGA |
| | | AGGCAGATAGCAGCCCCGTCAAGGCGGGAGTGGAGACCAC |
| | | CACACCCTCCAAACAAAGCAACAACAAGTACGCGGCCAGCA |
| | | GCTATCTGAGCCTGACGCCTGAGCAGTGGAAGTCCCACAG |
| | | AAGCTACAGCTGCCAGGTCACGCATGAAGGGAGCACCGTG |
| | | GAGAAGACAGTGGCCCCTACAGAATGTTCA |

Antibody 013

| | | |
|---|---|---|
| 313 | HCDR1 | GGTFRDYAIS |
| | (Combined) | |
| 314 | HCDR2 | GIIPAFGTANYAQKFQG |
| | (Combined) | |
| 315 | HCDR3 | EQDPEFGYGGYPYEAMDV |
| | (Combined) | |
| 316 | HCDR1 | DYAIS |
| | (Kabat) | |
| 317 | HCDR2 | GIIPAFGTANYAQKFQG |
| | (Kabat) | |
| 318 | HCDR3 | EQDPEFGYGGYPYEAMDV |
| | (Kabat) | |
| 319 | HCDR1 | GGTFRDY |
| | (Chothia) | |
| 320 | HCDR2 | IPAFGT |
| | (Chothia) | |
| 321 | HCDR3 | EQDPEFGYGGYPYEAMDV |
| | (Chothia) | |
| 322 | VH | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQDPEFGYGGYPYEAMPVWGQGTLVTVSS |
| 323 | VH DNA | CAGGTGCAGCTGGTGCAGTCAGGCGCCGAAGTGAAGAAACC |
| | | CGGCTCTAGCGTGAAAGTCAGCTGTAAAGCTAGTGGCGGCAC |
| | | CTTTAGAGACTACGCTATTAGCTGGGTCAGACAGGCCCCAGG |
| | | TCAGGGCCTGGAGTGGATGGGCGGAATTATCCCCGCCTTCGG |
| | | CACCGCTAACTACGCTCAGAAATTTCAGGGTAGAGTGACTATC |
| | | ACCGCCGACGAGTCTACTAGCACCGCCTATATGGAACTGTCTA |
| | | GCCTGAGATCAGAGGACACCGCCGTCTACTACTGCGCTAGAG |
| | | AGCAGGACCCCGAGTTCGGCTACGGCGGCTACCCCTACGAG |
| | | GCTATGGACGTGTGGGGTCAGGGCACCCTGGTCACCGTGTCT |
| | | AGC |
| 324 | Heavy Chain | QVQLVQSGAEVKKPGSSVKVSCKASGGTFRDYAISWVRQAPGQ |
| | | GLEWMGGIIPAFGTANYAQKFQGRVTITADESTSTAYMELSSLRS |
| | | EDTAVYYCAREQDPEFGYGGYPYEAMDVWGQGTLVTVSSASTK |
| | | GPSVFPLAPSSKSTSGGTAALGCLVKDYFPEPVTVSWNSGALTS |
| | | GVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISR |
| | | TPEVTCVVVDVSHEDPEVKFNWYVDGVEVHNAKTKPREEQYNS |
| | | TYRVVSVLTVLHQDWLNGKEYKCKVSNKALPAPIEKTISKAKGQP |
| | | REPQVYTLPPSREEMTKNQVSLTCLVKGFYPSDIAVEWESNGQP |
| | | ENNYKTTPPVLDSDGSFFLYSKLTVDKSRWQQGNVFSCSVMHE |
| | | ALHNHYTQKSLSLSPGK |
| 325 | Heavy Chain | CAGGTGCAGCTGGTGCAGTCAGGCGCCGAAGTGAAGAAACC |
| | DNA | CGGCTCTAGCGTGAAAGTCAGCTGTAAAGCTAGTGGCGGCAC |
| | | CTTTAGAGACTACGCTATTAGCTGGGTCAGACAGGCCCCAGG |
| | | TCAGGGCCTGGAGTGGATGGGCGGAATTATCCCCGCCTTCGG |
| | | CACCGCTAACTACGCTCAGAAATTTCAGGGTAGAGTGACTATC |
| | | ACCGCCGACGAGTCTACTAGCACCGCCTATATGGAACTGTCTA |
| | | GCCTGAGATCAGAGGACACCGCCGTCTACTACTGCGCTAGAG |
| | | AGCAGGACCCCGAGTTCGGCTACGGCGGCTACCCCTACGAG |
| | | GCTATGGACGTGTGGGGTCAGGGCACCCTGGTCACCGTGTCT |
| | | AGCGCTAGCACTAAGGGCCCAAGTGTGTTTCCCCTGGCCCCC |
| | | AGCAGCAAGTCTACTTCCGGCGGAACTGCTGCCCTGGGTTGC |
| | | CTGGTGAAGGACTACTTCCCCGAGCCCGTGACAGTGTCCTGG |
| | | AACTCTGGGGCTCTGACTTCCGGCGTGCACACCTTCCCCGCC |
| | | GTGCTGCAGAGCAGCGGCCTGTACAGCCTGAGCAGCGTGGT |
| | | GACAGTGCCCTCCAGCTCTCTGGGAACCCAGACCTATATCTG |
| | | CAACGTGAACCACAAGCCCAGCAACACCAAGGTGGACAAGAG |
| | | AGTGGAGCCCAAGAGCTGCGACAAGACCCACACCTGCCCCCC |
| | | CTGCCCAGCTCCAGAACTGCTGGGAGGGCCTTCCGTGTTCCT |
| | | GTTCCCCCCCAAGCCCAAGGACACCCTGATGATCAGCAGGAC |
| | | CCCCGAGGTGACCTGCGTGGTGGTGGACGTGTCCCACGAGG |
| | | ACCCAGAGGTGAAGTTCAACTGGTACGTGGACGGCGTGGAGG |
| | | TGCACAACGCCAAGACCAAGCCCAGAGAGGAGCAGTACAACA |
| | | GCACCTACAGGGTGGTGTCCGTGCTGACCGTGCTGCACCAGG |
| | | ACTGGCTGAACGGCAAAGAATACAAGTGCAAAGTCTCCAACAA |
| | | GGCCCTGCCAGCCCCAATCGAAAAGACAATCAGCAAGGCCAA |
| | | GGGCCAGCCACGGGAGCCCCAGGTGTACACCCTGCCCCCCA |
| | | GCCGGGAGGAGATGACCAAGAACCAGGTGTCCCTGACCTGTC |
| | | TGGTGAAGGGCTTCTACCCCAGCGATATCGCCGTGGAGTGGG |
| | | AGAGCAACGGCCAGCCCGAGAACAACTACAAGACCACCCCCC |
| | | CAGTGCTGGACAGCGACGGCAGCTTCTTCCTGTACAGCAAGC |
| | | TGACCGTGGACAAGTCCAGGTGGCAGCAGGGCAACGTGTTCA |
| | | GCTGCAGCGTGATGCACGAGGCCCTGCACAACCACTACACCC |
| | | AGAAGTCCCTGAGCCTGAGCCCCGGCAAG |
| 326 | LCDR1 | SGDNIPQHSVH |
| | (Combined) | |
| 327 | LCDR2 | DDTERPS |
| | (Combined) | |
| 328 | LCDR3 | SSWDSSMDSVV |
| | (Combined) | |
| 329 | LCDR1 | SGDNIPQHSVH |
| | (Kabat) | |
| 330 | LCDR2 | DDTERPS |
| | (Kabat) | |
| 331 | LCDR3 | SSWDSSMDSVV |
| | (Kabat) | |
| 332 | LCDR1 | DNIPQHS |
| | (Chothia) | |
| 333 | LCDR2 | DDT |
| | (Chothia) | |
| 334 | LCDR3 | WDSSMDSV |
| | (Chothia) | |
| 335 | VL | SYELTQPLSVSVALGQTARITCSGDNIPQHSVHWVQQKPGQAPV |
| | | LVIYDDTERPSGIPERFSGSNSGNTATLTISRAQAGDEADYYCSS |
| | | WDSSMDSVVFGGGTKLTVL |
| 336 | VH DNA | AGCTACGAGCTGACTCAGCCCCTGAGCGTCAGCGTGGCCCTG |
| | | GGTCAGACCGCTAGAATCACCTGTAGCGGCGATAATATCCCT |
| | | CAGCACTCAGTGCACTGGTATCAGCAGAAGCCCGGTCAGGCC |
| | | CCCGTGCTGGTGATCTACGACGACACCGAGCGGCCTAGCGG |
| | | AATCCCCGAGCGGTTTAGCGGCTCTAATAGCGGTAACACCGC |
| | | TACCCTGACTATCTCTAGGGCTCAGGCCGGCGACGAGGCCGA |
| | | CTACTACTGCTCTAGCTGGGATAGCTCTATGGATAGCGTGGTG |
| | | TTCGGCGGAGGCACTAAGCTGACCGTGCTG |
| 337 | Light Chain | SYELTQPLSVSVALGQTARITCSGDNIPQHSVHWVQQKPGQAPV |
| | | LVIYDDTERPSGIPERFSGSNSGNTATLTISRAQAGDEADYYCSS |
| | | WDSSMDSWFGGGTKLTVLGQPKAAPSVTLFPPSSEELQANKAT |
| | | LVCLISDFYPGAVTVAWKADSSPVKAGVETTTPSKQSNNKYAAS |
| | | SYLSLTPEQWKSHRSYSCQVTHEGSTVEKTVAPTECS |
| 338 | Light Chain | AGCTACGAGCTGACTCAGCCCCTGAGCGTCAGCGTGGCCCTG |
| | DNA | GGTCAGACCGCTAGAATCACCTGTAGCGGCGATAATATCCCT |
| | | CAGCACTCAGTGCACTGGTATCAGCAGAAGCCCGGTCAGGCC |
| | | CCCGTGCTGGTGATCTACGACGACACCGAGCGGCCTAGCGG |
| | | AATCCCCGAGCGGTTTAGCGGCTCTAATAGCGGTAACACCGC |
| | | TACCCTGACTATCTCTAGGGCTCAGGCCGGCGACGAGGCCGA |
| | | CTACTACTGCTCTAGCTGGGATAGCTCTATGGATAGCGTGGTG |
| | | TTCGGCGGAGGCACTAAGCTGACCGTGCTGGGTCAGCCTAAG |
| | | GCTGCCCCCAGCGTGACCCTGTTCCCCCCCAGCAGCGAGGA |
| | | GCTGCAGGCCAACAAGGCCACCCTGGTGTGCCTGATCAGCGA |
| | | CTTCTACCCAGGCGCCGTGACCGTGGCCTGGAAGGCCGACA |
| | | GCAGCCCCGTGAAGGCCGGCGTGGAGACCACCACCCCCAGC |
| | | AAGCAGAGCAACAACAAGTACGCCGCCAGCAGCTACCTGAGC |
| | | CTGACCCCCGAGCAGTGGAAGAGCCACAGGTCCTACAGCTGC |
| | | CAGGTGACCCACGAGGGCAGCACCGTGGAAAAGACCGTGGC |
| | | CCCAACCGAGTGCAGC |

Antibody 014

| | | |
|---|---|---|
| 339 | HCDR1 | GGTFRDYAIS |
| | (Combined) | |
| 340 | HCDR2 | GIIPAFGTANYAQKFQG |
| | (Combined) | |
| 341 | HCDR3 | EQDPEFGYGGYPYEAMDV |
| | (Combined) | |
| 342 | HCDR1 | DYAIS |
| | (Kabat) | |
| 343 | HCDR2 | GIIPAFGTANYAQKFQG |
| | (Kabat) | |
| 344 | HCDR3 | EQDPEFGYGGYPYEAMDV |
| | (Kabat) | |
| 345 | HCDR1 | GGTFRDY |
| | (Chothia) | |
| 346 | HCDR2 | IPAFGT |
| | (Chothia) | |
| 347 | HCDR3 | EQDPEFGYGGYPYEAMDV |
| | (Chothia) | |
| 348 | VH | QVQLVQSGAEVKKPGSSVKVSCKASGGTFRDYAISWVRQAPGQ |
| | | GLEWMGGIIPAFGTANYAQKFQGRVTITADESTSTAYMELSSLRS |
| | | EDTAVYYCAREQDPEFGYGGYPYEAMDVWGQGTLVTVSS |
| 349 | VH DNA | CAGGTGCAATTGGTGCAGAGCGGTGCCGAAGTGAAAAAACCG |
| | | GGCAGCAGCGTGAAAGTTAGCTGCAAAGCATCCGGAGGGAC |
| | | GTTTCGTGACTACGCTATCTCTTGGGTGCGCCAGGCCCCGGG |
| | | CCAGGGCCTCGAGTGGATGGGCGGTATCATCCCGGCTTTCGG |
| | | CACTGCGAACTACGCCCAGAAATTTCAGGGCCGGGTGACCAT |
| | | TACCGCCGATGAAAGCACCAGCACCGCCTATATGGAACTGAG |
| | | CAGCCTGCGCAGCGAAGATACGGCCGTGTATTATTGCGCGCG |
| | | TGAACAGGACCCGGAATTCGGTTACGGTGGTTACCCGTATGA |
| | | AGCTATGGATGTTTGGGGCCAAGGCACCCTGGTGACTGTTAG |
| | | CTCA |
| 350 | Heavy Chain | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQDPEFGYGGYPYEAMDVWGQGTLVTVSSASTK |
| | | GPSVFPLAPSSKSTSGGTAALGCLVKDYFPEPVTVSWNSGALTS |
| | | GVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISR |
| | | TPEVTCVVVDVSHEDPEVKFNWYVDGVEVHNAKTKPREEQYAS |
| | | TYRVVSVLTVLHQDWLNGKEYKCKVSNKALPAPIEKTISKAKGQP |
| | | REPQVYTLPPSREEMTKNQVSLTCLVKGFYPSDIAVEWESNGQP |
| | | ENNYKTTPPVLDSDGSFFLYSKLTVQKSRWQQGNVFSCSVMHE |
| | | ALHNHYTQKSLSLSPGK |
| 351 | Heavy Chain | CAGGTGCAATTGGTGCAGAGCGGTGCCGAAGTGAAAAAACCG |
| | DNA | GGCAGCAGCGTGAAAGTTAGCTGCAAAGCATCCGGAGGGAC |
| | | GTTTCGTGACTACGCTATCTCTTGGGTGCGCCAGGCCCCGGG |
| | | CCAGGGCCTCGAGTGGATGGGCGGTATCATCCCGGCTTTCGG |
| | | CACTGCGAACTACGCCCAGAAATTTCAGGGCCGGGTGACCAT |
| | | TACCGCCGATGAAAGCACCAGCACCGCCTATATGGAACTGAG |
| | | CAGCCTGCGCAGCGAAGATACGGCCGTGTATTATTGCGCGCG |
| | | TGAACAGGACCCGGAATTCGGTTACGGTGGTTACCCGTATGA |
| | | AGCTATGGATGTTTGGGGCCAAGGCACCCTGGTGACTGTTAG |
| | | CTCAGCCTCCACCAAGGGTCCATCGGTCTTCCCCCTGGCACC |
| | | CTCCTCCAAGAGCACCTCTGGGGGCACAGCGGCCCTGGGCT |
| | | GCCTGGTCAAGGACTACTTCCCCGAACCGGTGACGGTGTCGT |
| | | GGAACTCAGGCGCCCTGACCAGCGGCGTGCACACCTTCCCG |
| | | GCTGTCCTACAGTCCTCAGGACTCTACTCCCTCAGCAGCGTG |
| | | GTGACCGTGCCCTCCAGCAGCTTGGGCACCCAGACCTACATC |
| | | TGCAACGTGAATCACAAGCCCAGCAACACCAAGGTGGACAAG |
| | | AGAGTTGAGCCCAAATCTTGTGACAAAACTCACACATGCCCAC |
| | | CGTGCCCAGCACCTGAACTCCTGGGGGGACCGTCAGTCTTCC |
| | | TCTTCCCCCCAAAACCCAAGGACACCCTCATGATCTCCCGGAC |
| | | CCCTGAGGTCACATGCGTGGTGGTGGACGTGAGCCACGAAGA |
| | | CCCTGAGGTCAAGTTCAACTGGTACGTGGACGGCGTGGAGGT |
| | | GCATAATGCCAAGACAAAGCCGCGGGAGGAGCAGTACGCCA |
| | | GCACGTACCGGGTGGTCAGCGTCCTCACCGTCCTGCACCAGG |
| | | ACTGGCTGAATGGCAAGGAGTACAAGTGCAAGGTCTCCAACA |
| | | AAGCCCTCCCAGCCCCCATCGAGAAAACCATCTCCAAAGCCA |
| | | AAGGGCAGCCCCGAGAACCACAGGTGTACACCCTGCCCCCAT |
| | | CCCGGGAGGAGATGACCAAGAACCAGGTCAGCCTGACCTGC |
| | | CTGGTCAAAGGCTTCTATCCCAGCGACATCGCCGTGGAGTGG |
| | | GAGAGCAATGGGCAGCCGGAGAACAACTACAAGACCACGCCT |
| | | CCCGTGCTGGACTCCGACGGCTCCTTCTTCCTCTACAGCAAG |
| | | CTCACCGTGGACAAGAGCAGGTGGCAGCAGGGGAACGTCTTC |
| | | TCATGCTCCGTGATGCATGAGGCTCTGCACAACCACTACACG |
| | | CAGAAGAGCCTCTCCCTGTCTCCGGGTAAA |
| 352 | LCDR1 | SGDNIPQHSVH |
| | (Combined) | |
| 353 | LCDR2 | DDTERPS |
| | (Combined) | |
| 354 | LCDR3 | SSWDSSMDSVV |
| | (Combined) | |
| 355 | LCDR1 | SGDNIPQHSVH |
| | (Kabat) | |
| 356 | LCDR2 | DDTERPS |
| | (Kabat) | |
| 357 | LCDR3 | SSWDSSMDSVV |
| | (Kabat) | |
| 358 | LCDR1 | DNIPQHS |
| | (Chothia) | |
| 359 | LCDR2 | DDT |
| | (Chothia) | |
| 360 | LCDR3 | WDSSMDSV |
| | (Chothia) | |
| 361 | VL | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSVVFGGGTKLTVL |
| 362 | VH DNA | AGCTACGAACTGACCCAGCCGCTGAGCGTGAGCGTGGCCCT |
| | | GGGCCAGACCGCGAGGATTACCTGTAGCGGCGATAACATCCC |
| | | GCAGCATTCTGTTCATTGGTACCAGCAGAAACCGGGCCAGGC |
| | | GCCGGTGCTGGTGATCTACGACGACACTGAACGTCCGAGCGG |
| | | CATCCCGGAACGTTTTAGCGGATCCAACAGCGGCAACACCGC |
| | | GACCCTGACCATTAGCAGGGCCCAGGCGGGCGACGAAGCGG |
| | | ATTATTACTGCTCTTCTTGGGACTCTTCTATGGACTCTGTTGTG |
| | | TTTGGCGGCGGCACGAAGTTAACCGTCCTA |
| 363 | Light Chain | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSVVFGGGTKLTVLGQPKAAPSVTLFPPSSEELQANKAT |
| | | LVCLISDFYPGAVTVAWKADSSPVKAGVETTTPSKQSNNKYAAS |
| | | SYLSLTPEQWKSHRSYSCQVTHEGSTVEKTVAPTECS |
| 364 | Light Chain | AGCTACGAACTGACCCAGCCGCTGAGCGTGAGCGTGGCCCT |
| | DNA | GGGCCAGACCGCGAGGATTACCTGTAGCGGCGATAACATCCC |
| | | GCAGCATTCTGTTCATTGGTACCAGCAGAAACCGGGCCAGGC |
| | | GCCGGTGCTGGTGATCTACGACGACACTGAACGTCCGAGCGG |
| | | CATCCCGGAACGTTTTAGCGGATCCAACAGCGGCAACACCGC |
| | | GACCCTGACCATTAGCAGGGCCCAGGCGGGCGACGAAGCGG |
| | | ATTATTACTGCTCTTCTTGGGACTCTTCTATGGACTCTGTTGTG |
| | | TTTGGCGGCGGCACGAAGTTAACCGTCCTAGGTCAGCCCAAG |
| | | GCTGCCCCCTCGGTCACTCTGTTCCCGCCCTCCTCTGAGGAG |
| | | CTTCAAGCCAACAAGGCCACACTGGTGTGTCTCATAAGTGACT |
| | | TCTACCCGGGAGCCGTGACAGTGGCCTGGAAGGCAGATAGCA |
| | | GCCCCGTCAAGGCGGGAGTGGAGACCACCACACCCTCCAAA |
| | | CAAAGCAACAACAAGTACGCGGCCAGCAGCTATCTGAGCCTG |
| | | ACGCCTGAGCAGTGGAAGTCCCACAGAAGCTACAGCTGCCAG |
| | | GTCACGCATGAAGGGAGCACCGTGGAGAAGACAGTGGCCCC |
| | | TACAGAATGTTCA |

Antibody 015

| | | |
|---|---|---|
| 365 | HCDR1 | GGTFRDYAIS |
| | (Combined) | |
| 366 | HCDR2 | GIIPAFGTANYAQKFQG |
| | (Combined) | |
| 367 | HCDR3 | EQDPEAGYGGYPYEAMDV |
| | (Combined) | |
| 368 | HCDR1 | DYAIS |
| | (Kabat) | |
| 369 | HCDR2 | GIIPAFGTANYAQKFQG |
| | (Kabat) | |
| 370 | HCDR3 | EQDPEAGYGGYPYEAMDV |
| | (Kabat) | |
| 371 | HCDR1 | GGTFRDY |
| | (Chothia) | |
| 372 | HCDR2 | IPAFGT |
| | (Chothia) | |
| 373 | HCDR3 | EQDPEAGYGGYPYEAMDV |
| | (Chothia) | |
| 374 | VH | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQDPEAGYGGYPYEAMDVWGQGTLVTVSS |
| 375 | VH DNA | CAGGTGCAGCTGGTGCAGTCAGGCGCCGAAGTGAAGAAACC |
| | | CGGCTCTAGCGTGAAAGTCAGCTGTAAAGCTAGTGGCGGCAC |
| | | CTTTAGAGACTACGCTATTAGCTGGGTCAGACAGGCCCCAGG |
| | | TCAGGGCCTGGAGTGGATGGGCGGAATTATCCCCGCCTTCGG |
| | | CACCGCTAACTACGCTCAGAAATTTCAGGGTAGAGTGACTATC |
| | | ACCGCCGACGAGTCTACTAGCACCGCCTATATGGAACTGTCTA |
| | | GCCTGAGATCAGAGGACACCGCCGTCTACTACTGCGCTAGAG |
| | | AGCAGGACCCCGAGGCCGGCTACGGCGGCTACCCCTACGAG |
| | | GCTATGGACGTGTGGGGTCAGGGCACCCTGGTCACCGTGTCT |
| | | AGC |
| 376 | Heavy Chain | QVQLVQSGAEVKKPGSSVKVSCKASGGTFRDYAISWVRQAPGQ |
| | | GLEWMGGIIPAFGTANYAQKFQGRVTITADESTSTAYMELSSLRS |
| | | EDTAVYYCAREQDPEAGYGGYPYEAMDVWGQGTLVTVSSASTK |
| | | GPSVFPLAPSSKSTSGGTAALGCLVKDYFPEPVTVSWNSGALTS |
| | | GVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISR |
| | | TPEVTCVVVDVSHEDPEVKFNWYVDGVEVHNAKTKPREEQYNS |
| | | TYRVVSVLTVLHQDWLNGKEYKCKVSNKALPAPIEKTISKAKGQP |
| | | REPQVYTLPPSREEMTKNQVSLTCLVKGFYPSDIAVEWESNGQP |
| | | ENNYKTTPPVLDSDGSFFLYSKLTVDKSRWQQGNVFSCSVMHE |
| | | ALHNHYTQKSLSLSPGK |
| 377 | Heavy Chain | CAGGTGCAGCTGGTGCAGTCAGGCGCCGAAGTGAAGAAACC |
| | DNA | CGGCTCTAGCGTGAAAGTCAGCTGTAAAGCTAGTGGCGGCAC |
| | | CTTTAGAGACTACGCTATTAGCTGGGTCAGACAGGCCCCAGG |
| | | TCAGGGCCTGGAGTGGATGGGCGGAATTATCCCCGCCTTCGG |
| | | CACCGCTAACTACGCTCAGAAATTTCAGGGTAGAGTGACTATC |
| | | ACCGCCGACGAGTCTACTAGCACCGCCTATATGGAACTGTCTA |
| | | GCCTGAGATCAGAGGACACCGCCGTCTACTACTGCGCTAGAG |
| | | AGCAGGACCCCGAGGCCGGCTACGGCGGCTACCCCTACGAG |
| | | GCTATGGACGTGTGGGGTCAGGGCACCCTGGTCACCGTGTCT |
| | | AGCGCTAGCACTAAGGGCCCAAGTGTGTTTCCCCTGGCCCCC |
| | | AGCAGCAAGTCTACTTCCGGCGGAACTGCTGCCCTGGGTTGC |
| | | CTGGTGAAGGACTACTTCCCCGAGCCCGTGACAGTGTCCTGG |
| | | AACTCTGGGGCTCTGACTTCCGGCGTGCACACCTTCCCCGCC |
| | | GTGCTGCAGAGCAGCGGCCTGTACAGCCTGAGCAGCGTGGT |
| | | GACAGTGCCCTCCAGCTCTCTGGGAACCCAGACCTATATCTG |
| | | CAACGTGAACCACAAGCCCAGCAACACCAAGGTGGACAAGAG |
| | | AGTGGAGCCCAAGAGCTGCGACAAGACCCACACCTGCCCCCC |
| | | CTGCCCAGCTCCAGAACTGCTGGGAGGGCCTTCCGTGTTCCT |
| | | GTTCCCCCCCAAGCCCAAGGACACCCTGATGATCAGCAGGAC |
| | | CCCCGAGGTGACCTGCGTGGTGGTGGACGTGTCCCACGAGG |
| | | ACCCAGAGGTGAAGTTCAACTGGTACGTGGACGGCGTGGAGG |
| | | TGCACAACGCCAAGACCAAGCCCAGAGAGGAGCAGTACAACA |
| | | GCACCTACAGGGTGGTGTCCGTGCTGACCGTGCTGCACCAGG |
| | | ACTGGCTGAACGGCAAAGAATACAAGTGCAAAGTCTCCAACAA |
| | | GGCCCTGCCAGCCCCAATCGAAAAGACAATCAGCAAGGCCAA |
| | | GGGCCAGCCACGGGAGCCCCAGGTGTACACCCTGCCCCCCA |
| | | GCCGGGAGGAGATGACCAAGAACCAGGTGTCCCTGACCTGTC |
| | | TGGTGAAGGGCTTCTACCCCAGCGATATCGCCGTGGAGTGGG |
| | | AGAGCAACGGCCAGCCCGAGAACAACTACAAGACCACCCCCC |
| | | CAGTGCTGGACAGCGACGGCAGCTTCTTCCTGTACAGCAAGC |
| | | TGACCGTGGACAAGTCCAGGTGGCAGCAGGGCAACGTGTTCA |
| | | GCTGCAGCGTGATGCACGAGGCCCTGCACAACCACTACACCC |
| | | AGAAGTCCCTGAGCCTGAGCCCCGGCAAG |
| 378 | LCDR1 | SGDNIPQHSVH |
| | (Combined) | |
| 379 | LCDR2 | DDTERPS |
| | (Combined) | |
| 380 | LCDR3 | SSWDSSMDSVV |
| | (Combined) | |
| 381 | LCDR1 | SGDNIPQHSVH |
| | (Kabat) | |
| 382 | LCDR2 | DDTERPS |
| | (Kabat) | |
| 383 | LCDR3 | SSWDSSMDSVV |
| | (Kabat) | |
| 384 | LCDR1 | DNIPQHS |
| | (Chothia) | |
| 385 | LCDR2 | DDT |
| | (Chothia) | |
| 386 | LCDR3 | WDSSMDSV |
| | (Chothia) | |
| 387 | VL | SYELTQPLSVSVALGQTARITCSGDNIPQHSVHWVQQKPGQAPV |
| | | LVIYDDTERPSGIPERFSGSNSGNTATLTISRAQAGDEADYYCSS |
| | | WDSSMDSVVFGGGTKLTVL |
| 388 | VH DNA | AGCTACGAGCTGACTCAGCCCCTGAGCGTCAGCGTGGCCCTG |
| | | GGTCAGACCGCTAGAATCACCTGTAGCGGCGATAATATCCCT |
| | | CAGCACTCAGTGCACTGGTATCAGCAGAAGCCCGGTCAGGCC |
| | | CCCGTGCTGGTGATCTACGACGACACCGAGCGGCCTAGCGG |
| | | AATCCCCGAGCGGTTTAGCGGCTCTAATAGCGGTAACACCGC |
| | | TACCCTGACTATCTCTAGGGCTCAGGCCGGCGACGAGGCCGA |
| | | CTACTACTGCTCTAGCTGGGATAGCTCTATGGATAGCGTGGTG |
| | | TTCGGCGGAGGCACTAAGCTGACCGTGCTG |
| 389 | Light Chain | SYELTQPLSVSVALGQTARITCSGDNIPQHSVHWVQQKPGQAPV |
| | | LVIYDDTERPSGIPERFSGSNSGNTATLTISRAQAGDEADYYCSS |
| | | WDSSMDSWFGGGTKLTVLGQPKAAPSVTLFPPSSEELQANKAT |
| | | LVCLISDFYPGAVTVAWKADSSPVKAGVETTTPSKQSNNKYAAS |
| | | SYLSLTPEQWKSHRSYSCQVTHEGSTVEKTVAPTECS |
| 390 | Light Chain | AGCTACGAGCTGACTCAGCCCCTGAGCGTCAGCGTGGCCCTG |
| | DNA | GGTCAGACCGCTAGAATCACCTGTAGCGGCGATAATATCCCT |
| | | CAGCACTCAGTGCACTGGTATCAGCAGAAGCCCGGTCAGGCC |
| | | CCCGTGCTGGTGATCTACGACGACACCGAGCGGCCTAGCGG |
| | | AATCCCCGAGCGGTTTAGCGGCTCTAATAGCGGTAACACCGC |
| | | TACCCTGACTATCTCTAGGGCTCAGGCCGGCGACGAGGCCGA |
| | | CTACTACTGCTCTAGCTGGGATAGCTCTATGGATAGCGTGGTG |
| | | TTCGGCGGAGGCACTAAGCTGACCGTGCTGGGTCAGCCTAAG |
| | | GCTGCCCCCAGCGTGACCCTGTTCCCCCCCAGCAGCGAGGA |
| | | GCTGCAGGCCAACAAGGCCACCCTGGTGTGCCTGATCAGCGA |
| | | CTTCTACCCAGGCGCCGTGACCGTGGCCTGGAAGGCCGACA |
| | | GCAGCCCCGTGAAGGCCGGCGTGGAGACCACCACCCCCAGC |
| | | AAGCAGAGCAACAACAAGTACGCCGCCAGCAGCTACCTGAGC |
| | | CTGACCCCCGAGCAGTGGAAGAGCCACAGGTCCTACAGCTGC |
| | | CAGGTGACCCACGAGGGCAGCACCGTGGAAAAGACCGTGGC |
| | | CCCAACCGAGTGCAGC |

Antibody 016

| | | |
|---|---|---|
| 391 | HCDR1 | GGTFRDYAIS |
| | (Combined) | |
| 392 | HCDR2 | GIIPAFGTANYAQKFQG |
| | (Combined) | |
| 393 | HCDR3 | EQDPEAGYGGYPYEAMDV |
| | (Combined) | |
| 394 | HCDR1 | DYAIS |
| | (Kabat) | |
| 395 | HCDR2 | GIIPAFGTANYAQKFQG |
| | (Kabat) | |
| 396 | HCDR3 | EQDPEAGYGGYPYEAMDV |
| | (Kabat) | |
| 397 | HCDR1 | GGTFRDY |
| | (Chothia) | |
| 398 | HCDR2 | IPAFGT |
| | (Chothia) | |
| 399 | HCDR3 | EQDPEAGYGGYPYEAMDV |
| | (Chothia) | |
| 400 | VH | QVQLVQSGAEVKKPGSSVKVSCKASGGTFRDYAISWVRQAPGQ |
| | | GLEWMGGIIPAFGTANYAQKFQGRVTITADESTSTAYMELSSLRS |
| | | EDTAVYYCAREQDPEAGYGGYPYEAMDVWGQGTLVTVSS |
| 401 | VH DNA | CAGGTGCAATTGGTGCAGAGCGGTGCCGAAGTGAAAAAACCG |
| | | GGCAGCAGCGTGAAAGTTAGCTGCAAAGCATCCGGAGGGAC |
| | | GTTTCGTGACTACGCTATCTCTTGGGTGCGCCAGGCCCCGGG |
| | | CCAGGGCCTCGAGTGGATGGGCGGTATCATCCCGGCTTTCGG |
| | | CACTGCGAACTACGCCCAGAAATTTCAGGGCCGGGTGACCAT |
| | | TACCGCCGATGAAAGCACCAGCACCGCCTATATGGAACTGAG |
| | | CAGCCTGCGCAGCGAAGATACGGCCGTGTATTATTGCGCGCG |
| | | TGAACAGGACCCGGAAGCCGGTTACGGTGGTTACCCGTATGA |
| | | AGCTATGGATGTTTGGGGCCAAGGCACCCTGGTGACTGTTAG |
| | | CTCA |
| 402 | Heavy Chain | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQDPEAGYGGYPYEAMDVWGQGTLVTVSSASTK |
| | | GPSVFPLAPSSKSTSGGTAALGCLVKDYFPEPVTVSWNSGALTS |
| | | GVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISR |
| | | TPEVTCVVVDVSHEDPEVKFNWYVDGVEVHNAKTKPREEQYAS |
| | | TYRVVSVLTVLHQDWLNGKEYKCKVSNKALPAPIEKTISKAKGQP |
| | | REPQVYTLPPSREEMTKNQVSLTCLVKGFYPSDIAVEWESNGQP |
| | | ENNYKTTPPVLDSDGSFFLYSKLTVDKSRWQQGNVFSCSVMHE |
| | | ALHNHYTQKSLSLSPGK |
| 403 | Heavy Chain | CAGGTGCAATTGGTGCAGAGCGGTGCCGAAGTGAAAAAACCG |
| | DNA | GGCAGCAGCGTGAAAGTTAGCTGCAAAGCATCCGGAGGGAC |
| | | GTTTCGTGACTACGCTATCTCTTGGGTGCGCCAGGCCCCGGG |
| | | CCAGGGCCTCGAGTGGATGGGCGGTATCATCCCGGCTTTCGG |
| | | CACTGCGAACTACGCCCAGAAATTTCAGGGCCGGGTGACCAT |
| | | TACCGCCGATGAAAGCACCAGCACCGCCTATATGGAACTGAG |
| | | CAGCCTGCGCAGCGAAGATACGGCCGTGTATTATTGCGCGCG |
| | | TGAACAGGACCCGGAAGCCGGTTACGGTGGTTACCCGTATGA |
| | | AGCTATGGATGTTTGGGGCCAAGGCACCCTGGTGACTGTTAG |
| | | CTCAGCCTCCACCAAGGGTCCATCGGTCTTCCCCCTGGCACC |
| | | CTCCTCCAAGAGCACCTCTGGGGGCACAGCGGCCCTGGGCT |
| | | GCCTGGTCAAGGACTACTTCCCCGAACCGGTGACGGTGTCGT |
| | | GGAACTCAGGCGCCCTGACCAGCGGCGTGCACACCTTCCCG |
| | | GCTGTCCTACAGTCCTCAGGACTCTACTCCCTCAGCAGCGTG |
| | | GTGACCGTGCCCTCCAGCAGCTTGGGCACCCAGACCTACATC |
| | | TGCAACGTGAATCACAAGCCCAGCAACACCAAGGTGGACAAG |
| | | AGAGTTGAGCCCAAATCTTGTGACAAAACTCACACATGCCCAC |
| | | CGTGCCCAGCACCTGAACTCCTGGGGGGACCGTCAGTCTTCC |
| | | TCTTCCCCCCAAAACCCAAGGACACCCTCATGATCTCCCGGAC |
| | | CCCTGAGGTCACATGCGTGGTGGTGGACGTGAGCCACGAAGA |
| | | CCCTGAGGTCAAGTTCAACTGGTACGTGGACGGCGTGGAGGT |
| | | GCATAATGCCAAGACAAAGCCGCGGGAGGAGCAGTACGCCA |
| | | GCACGTACCGGGTGGTCAGCGTCCTCACCGTCCTGCACCAGG |
| | | ACTGGCTGAATGGCAAGGAGTACAAGTGCAAGGTCTCCAACA |
| | | AAGCCCTCCCAGCCCCCATCGAGAAAACCATCTCCAAAGCCA |
| | | AAGGGCAGCCCCGAGAACCACAGGTGTACACCCTGCCCCCAT |
| | | CCCGGGAGGAGATGACCAAGAACCAGGTCAGCCTGACCTGC |
| | | CTGGTCAAAGGCTTCTATCCCAGCGACATCGCCGTGGAGTGG |
| | | GAGAGCAATGGGCAGCCGGAGAACAACTACAAGACCACGCCT |
| | | CCCGTGCTGGACTCCGACGGCTCCTTCTTCCTCTACAGCAAG |
| | | CTCACCGTGGACAAGAGCAGGTGGCAGCAGGGGAACGTCTTC |
| | | TCATGCTCCGTGATGCATGAGGCTCTGCACAACCACTACACG |
| | | CAGAAGAGCCTCTCCCTGTCTCCGGGTAAA |
| 404 | LCDR1 | SGDNIPQHSVH |
| | (Combined) | |
| 405 | LCDR2 | DDTERPS |
| | (Combined) | |
| 406 | LCDR3 | SSWDSSMDSVV |
| | (Combined) | |
| 407 | LCDR1 | SGDNIPQHSVH |
| | (Kabat) | |
| 408 | LCDR2 | DDTERPS |
| | (Kabat) | |
| 409 | LCDR3 | SSWDSSMDSVV |
| | (Kabat) | |
| 410 | LCDR1 | DNIPQHS |
| | (Chothia) | |
| 411 | LCDR2 | DDT |
| | (Chothia) | |
| 412 | LCDR3 | WDSSMDSV |
| | (Chothia) | |
| 413 | VL | SYELTQPLSVSVALGQTARITCSGDNIPQHSVHVWQQKPGQAPV |
| | | LVIYDDTERPSGIPERFSGSNSGNTATLTISRAQAGDEADYYCSS |
| | | WDSSMDSWFGGGTKLTVL |
| 414 | VH DNA | AGCTACGAACTGACCCAGCCGCTGAGCGTGAGCGTGGCCCT |
| | | GGGCCAGACCGCGAGGATTACCTGTAGCGGCGATAACATCCC |
| | | GCAGCATTCTGTTCATTGGTACCAGCAGAAACCGGGCCAGGC |
| | | GCCGGTGCTGGTGATCTACGACGACACTGAACGTCCGAGCGG |
| | | CATCCCGGAACGTTTTAGCGGATCCAACAGCGGCAACACCGC |
| | | GACCCTGACCATTAGCAGGGCCCAGGCGGGCGACGAAGCGG |
| | | ATTATTACTGCTCTTCTTGGGACTCTTCTATGGACTCTGTTGTG |
| | | TTTGGCGGCGGCACGAAGTTAACCGTCCTA |
| 415 | Light Chain | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSWFGGGTKLTVLGQPKAAPSVTLFPPSSEELQANKAT |
| | | LVCLISDFYPGAVTVAWKADSSPVKAGVETTTPSKQSNNKYAAS |
| | | SYLSLTPEQWKSHRSYSCQVTHEGSTVEKTVAPTECS |
| 416 | Light Chain | AGCTACGAACTGACCCAGCCGCTGAGCGTGAGCGTGGCCCT |
| | DNA | GGGCCAGACCGCGAGGATTACCTGTAGCGGCGATAACATCCC |
| | | GCAGCATTCTGTTCATTGGTACCAGCAGAAACCGGGCCAGGC |
| | | GCCGGTGCTGGTGATCTACGACGACACTGAACGTCCGAGCGG |
| | | CATCCCGGAACGTTTTAGCGGATCCAACAGCGGCAACACCGC |
| | | GACCCTGACCATTAGCAGGGCCCAGGCGGGCGACGAAGCGG |
| | | ATTATTACTGCTCTTCTTGGGACTCTTCTATGGACTCTGTTGTG |
| | | TTTGGCGGCGGCACGAAGTTAACCGTCCTAGGTCAGCCCAAG |
| | | GCTGCCCCCTCGGTCACTCTGTTCCCGCCCTCCTCTGAGGAG |
| | | CTTCAAGCCAACAAGGCCACACTGGTGTGTCTCATAAGTGACT |
| | | TCTACCCGGGAGCCGTGACAGTGGCCTGGAAGGCAGATAGCA |
| | | GCCCCGTCAAGGCGGGAGTGGAGACCACCACACCCTCCAAA |
| | | CAAAGCAACAACAAGTACGCGGCCAGCAGCTATCTGAGCCTG |
| | | ACGCCTGAGCAGTGGAAGTCCCACAGAAGCTACAGCTGCCAG |
| | | GTCACGCATGAAGGGAGCACCGTGGAGAAGACAGTGGCCCC |
| | | TACAGAATGTTCA |

Antibody 017

| | | |
|---|---|---|
| 417 | HCDR1 | GGTFRDYAIS |
| | (Combined) | |
| 418 | HCDR2 | GIIPAFGTANYAQKFQG |
| | (Combined) | |
| 419 | HCDR3 | EQDPESGYGGYPYEAMDV |
| | (Combined) | |
| 420 | HCDR1 | DYAIS |
| | (Kabat) | |
| 421 | HCDR2 | GIIPAFGTANYAQKFQG |
| | (Kabat) | |
| 422 | HCDR3 | EQDPESGYGGYPYEAMDV |
| | (Kabat) | |
| 423 | HCDR1 | GGTFRDY |
| | (Chothia) | |
| 424 | HCDR2 | IPAFGT |
| | (Chothia) | |
| 425 | HCDR3 | EQDPESGYGGYPYEAMDV |
| | (Chothia) | |
| 426 | VH | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQDPESGYGGYPYEAMDVWGQGTLVTVSS |
| 427 | VH DNA | CAGGTGCAGCTGGTGCAGTCAGGCGCCGAAGTGAAGAAACC |
| | | CGGCTCTAGCGTGAAAGTCAGCTGTAAAGCTAGTGGCGGCAC |
| | | CTTTAGAGACTACGCTATTAGCTGGGTCAGACAGGCCCCAGG |
| | | TCAGGGCCTGGAGTGGATGGGCGGAATTATCCCCGCCTTCGG |
| | | CACCGCTAACTACGCTCAGAAATTTCAGGGTAGAGTGACTATC |
| | | ACCGCCGACGAGTCTACTAGCACCGCCTATATGGAACTGTCTA |
| | | GCCTGAGATCAGAGGACACCGCCGTCTACTACTGCGCTAGAG |
| | | AGCAGGACCCCGAGTCCGGCTACGGCGGCTACCCCTACGAG |
| | | GCTATGGACGTGTGGGGTCAGGGCACCCTGGTCACCGTGTCT |
| | | AGC |
| 428 | Heavy Chain | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQDPESGYGGYPYEAMDVWGQGTLVTVSSASTK |
| | | GPSVFPLAPSSKSTSGGTAALGCLVKDYFPEPVTVSWNSGALTS |
| | | GVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISR |
| | | TPEVTCVVVDVSHEDPEVKFNWYVDGVEVHNAKTKPREEQYNS |
| | | TYRVVSVLTVLHQDWLNGKEYKCKVSNKALPAPIEKTISKAKGQP |
| | | REPQVYTLPPSREEMTKNQVSLTCLVKGFYPSDIAVEWESNGQP |
| | | ENNYKTTPPVLDSDGSFFLYSKLTVDKSRWQQGNVFSCSVMHE |
| | | ALHNHYTQKSLSLSPGK |
| 429 | Heavy Chain | CAGGTGCAGCTGGTGCAGTCAGGCGCCGAAGTGAAGAAACC |
| | DNA | CGGCTCTAGCGTGAAAGTCAGCTGTAAAGCTAGTGGCGGCAC |
| | | CTTTAGAGACTACGCTATTAGCTGGGTCAGACAGGCCCCAGG |
| | | TCAGGGCCTGGAGTGGATGGGCGGAATTATCCCCGCCTTCGG |
| | | CACCGCTAACTACGCTCAGAAATTTCAGGGTAGAGTGACTATC |
| | | ACCGCCGACGAGTCTACTAGCACCGCCTATATGGAACTGTCTA |
| | | GCCTGAGATCAGAGGACACCGCCGTCTACTACTGCGCTAGAG |
| | | AGCAGGACCCCGAGTCCGGCTACGGCGGCTACCCCTACGAG |
| | | GCTATGGACGTGTGGGGTCAGGGCACCCTGGTCACCGTGTCT |
| | | AGCGCTAGCACTAAGGGCCCAAGTGTGTTTCCCCTGGCCCCC |
| | | AGCAGCAAGTCTACTTCCGGCGGAACTGCTGCCCTGGGTTGC |
| | | CTGGTGAAGGACTACTTCCCCGAGCCCGTGACAGTGTCCTGG |
| | | AACTCTGGGGCTCTGACTTCCGGCGTGCACACCTTCCCCGCC |
| | | GTGCTGCAGAGCAGCGGCCTGTACAGCCTGAGCAGCGTGGT |
| | | GACAGTGCCCTCCAGCTCTCTGGGAACCCAGACCTATATCTG |
| | | CAACGTGAACCACAAGCCCAGCAACACCAAGGTGGACAAGAG |
| | | AGTGGAGCCCAAGAGCTGCGACAAGACCCACACCTGCCCCCC |
| | | CTGCCCAGCTCCAGAACTGCTGGGAGGGCCTTCCGTGTTCCT |
| | | GTTCCCCCCCAAGCCCAAGGACACCCTGATGATCAGCAGGAC |
| | | CCCCGAGGTGACCTGCGTGGTGGTGGACGTGTCCCACGAGG |
| | | ACCCAGAGGTGAAGTTCAACTGGTACGTGGACGGCGTGGAGG |
| | | TGCACAACGCCAAGACCAAGCCCAGAGAGGAGCAGTACAACA |
| | | GCACCTACAGGGTGGTGTCCGTGCTGACCGTGCTGCACCAGG |
| | | ACTGGCTGAACGGCAAAGAATACAAGTGCAAAGTCTCCAACAA |
| | | GGCCCTGCCAGCCCCAATCGAAAAGACAATCAGCAAGGCCAA |
| | | GGGCCAGCCACGGGAGCCCCAGGTGTACACCCTGCCCCCCA |
| | | GCCGGGAGGAGATGACCAAGAACCAGGTGTCCCTGACCTGTC |
| | | TGGTGAAGGGCTTCTACCCCAGCGATATCGCCGTGGAGTGGG |
| | | AGAGCAACGGCCAGCCCGAGAACAACTACAAGACCACCCCCC |
| | | CAGTGCTGGACAGCGACGGCAGCTTCTTCCTGTACAGCAAGC |
| | | TGACCGTGGACAAGTCCAGGTGGCAGCAGGGCAACGTGTTCA |
| | | GCTGCAGCGTGATGCACGAGGCCCTGCACAACCACTACACCC |
| | | AGAAGTCCCTGAGCCTGAGCCCCGGCAAG |
| 430 | LCDR1 | SGDNIPQHSVH |
| | (Combined) | |
| 431 | LCDR2 | DDTERPS |
| | (Combined) | |
| 432 | LCDR3 | SSWDSSMDSVV |
| | (Combined) | |
| 433 | LCDR1 | SGDNIPQHSVH |
| | (Kabat) | |
| 434 | LCDR2 | DDTERPS |
| | (Kabat) | |
| 435 | LCDR3 | SSWDSSMDSVV |
| | (Kabat) | |
| 436 | LCDR1 | DNIPQHS |
| | (Chothia) | |
| 437 | LCDR2 | DDT |
| | (Chothia) | |
| 438 | LCDR3 | WDSSMDSV |
| | (Chothia) | |
| 439 | VL | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSVVFGGGTKLTVL |
| 440 | VH DNA | AGCTACGAGCTGACTCAGCCCCTGAGCGTCAGCGTGGCCCTG |
| | | GGTCAGACCGCTAGAATCACCTGTAGCGGCGATAATATCCCT |
| | | CAGCACTCAGTGCACTGGTATCAGCAGAAGCCCGGTCAGGCC |
| | | CCCGTGCTGGTGATCTACGACGACACCGAGCGGCCTAGCGG |
| | | AATCCCCGAGCGGTTTAGCGGCTCTAATAGCGGTAACACCGC |
| | | TACCCTGACTATCTCTAGGGCTCAGGCCGGCGACGAGGCCGA |
| | | CTACTACTGCTCTAGCTGGGATAGCTCTATGGATAGCGTGGTG |
| | | TTCGGCGGAGGCACTAAGCTGACCGTGCTG |
| 441 | Light Chain | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSVVFGGGTKLTVLGQPKAAPSVTLFPPSSEELQANKAT |
| | | LVCLISDFYPGAVTVAWKADSSPVKAGVETTTPSKQSNNKYAAS |
| | | SYLSLTPEQWKSHRSYSCQVTHEGSTVEKTVAPTECS |
| 442 | Light Chain | AGCTACGAGCTGACTCAGCCCCTGAGCGTCAGCGTGGCCCTG |
| | DNA | GGTCAGACCGCTAGAATCACCTGTAGCGGCGATAATATCCCT |
| | | CAGCACTCAGTGCACTGGTATCAGCAGAAGCCCGGTCAGGCC |
| | | CCCGTGCTGGTGATCTACGACGACACCGAGCGGCCTAGCGG |
| | | AATCCCCGAGCGGTTTAGCGGCTCTAATAGCGGTAACACCGC |
| | | TACCCTGACTATCTCTAGGGCTCAGGCCGGCGACGAGGCCGA |
| | | CTACTACTGCTCTAGCTGGGATAGCTCTATGGATAGCGTGGTG |
| | | TTCGGCGGAGGCACTAAGCTGACCGTGCTGGGTCAGCCTAAG |
| | | GCTGCCCCCAGCGTGACCCTGTTCCCCCCCAGCAGCGAGGA |
| | | GCTGCAGGCCAACAAGGCCACCCTGGTGTGCCTGATCAGCGA |
| | | CTTCTACCCAGGCGCCGTGACCGTGGCCTGGAAGGCCGACA |
| | | GCAGCCCCGTGAAGGCCGGCGTGGAGACCACCACCCCCAGC |
| | | AAGCAGAGCAACAACAAGTACGCCGCCAGCAGCTACCTGAGC |
| | | CTGACCCCCGAGCAGTGGAAGAGCCACAGGTCCTACAGCTGC |
| | | CAGGTGACCCACGAGGGCAGCACCGTGGAAAAGACCGTGGC |
| | | CCCAACCGAGTGCAGC |

Antibody 018

| | | |
|---|---|---|
| 443 | HCDR1 | GGTFRDYAIS |
| | (Combined) | |
| 444 | HCDR2 | GIIPAFGTANYAQKFQG |
| | (Combined) | |
| 445 | HCDR3 | EQDPESGYGGYPYEAMDV |
| | (Combined) | |
| 446 | HCDR1 | DYAIS |
| | (Kabat) | |
| 447 | HCDR2 | GIIPAFGTANYAQKFQG |
| | (Kabat) | |
| 448 | HCDR3 | EQDPESGYGGYPYEAMDV |
| | (Kabat) | |
| 449 | HCDR1 | GGTFRDY |
| | (Chothia) | |
| 450 | HCDR2 | IPAFGT |
| | (Chothia) | |
| 451 | HCDR3 | EQDPESGYGGYPYEAMDV |
| | (Chothia) | |
| 452 | VH | QVQLVQSGAEVKKPGSSVKVSCKASGGTFRDYAISWVRQAPGQ |
| | | GLEWMGGIIPAFGTANYAQKFQGRVTITADESTSTAYMELSSLRS |
| | | EDTAVYYCAREQDPESGYGGYPYEAMDVWGQGTLVTVSS |
| 453 | VH DNA | CAGGTGCAATTGGTGCAGAGCGGTGCCGAAGTGAAAAAACCG |
| | | GGCAGCAGCGTGAAAGTTAGCTGCAAAGCATCCGGAGGGAC |
| | | GTTTCGTGACTACGCTATCTCTTGGGTGCGCCAGGCCCCGGG |
| | | CCAGGGCCTCGAGTGGATGGGCGGTATCATCCCGGCTTTCGG |
| | | CACTGCGAACTACGCCCAGAAATTTCAGGGCCGGGTGACCAT |
| | | TACCGCCGATGAAAGCACCAGCACCGCCTATATGGAACTGAG |
| | | CAGCCTGCGCAGCGAAGATACGGCCGTGTATTATTGCGCGCG |
| | | TGAACAGGACCCGGAAAGCGGTTACGGTGGTTACCCGTATGA |
| | | AGCTATGGATGTTTGGGGCCAAGGCACCCTGGTGACTGTTAG |
| | | CTCA |
| 454 | Heavy Chain | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQDPESGYGGYPYEAMDVWGQGTLVTVSSASTK |
| | | GPSVFPLAPSSKSTSGGTAALGCLVKDYFPEPVTVSWNSGALTS |
| | | GVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISR |
| | | TPEVTCVVVDVSHEDPEVKFNWYVDGVEVHNAKTKPREEQYAS |
| | | TYRVVSVLTVLHQDWLNGKEYKCKVSNKALPAPIEKTISKAKGQP |
| | | REPQVYTLPPSREEMTKNQVSLTCLVKGFYPSDIAVEWESNGQP |
| | | ENNYKTTPPVLDSDGSFFLYSKLTVQKSRWQQGNVFSCSVMHE |
| | | ALHNHYTQKSLSLSPGK |
| 455 | Heavy Chain | CAGGTGCAATTGGTGCAGAGCGGTGCCGAAGTGAAAAAACCG |
| | DNA | GGCAGCAGCGTGAAAGTTAGCTGCAAAGCATCCGGAGGGAC |
| | | GTTTCGTGACTACGCTATCTCTTGGGTGCGCCAGGCCCCGGG |
| | | CCAGGGCCTCGAGTGGATGGGCGGTATCATCCCGGCTTTCGG |
| | | CACTGCGAACTACGCCCAGAAATTTCAGGGCCGGGTGACCAT |
| | | TACCGCCGATGAAAGCACCAGCACCGCCTATATGGAACTGAG |
| | | CAGCCTGCGCAGCGAAGATACGGCCGTGTATTATTGCGCGCG |
| | | TGAACAGGACCCGGAAAGCGGTTACGGTGGTTACCCGTATGA |
| | | AGCTATGGATGTTTGGGGCCAAGGCACCCTGGTGACTGTTAG |
| | | CTCAGCCTCCACCAAGGGTCCATCGGTCTTCCCCCTGGCACC |
| | | CTCCTCCAAGAGCACCTCTGGGGGCACAGCGGCCCTGGGCT |
| | | GCCTGGTCAAGGACTACTTCCCCGAACCGGTGACGGTGTCGT |
| | | GGAACTCAGGCGCCCTGACCAGCGGCGTGCACACCTTCCCG |
| | | GCTGTCCTACAGTCCTCAGGACTCTACTCCCTCAGCAGCGTG |
| | | GTGACCGTGCCCTCCAGCAGCTTGGGCACCCAGACCTACATC |
| | | TGCAACGTGAATCACAAGCCCAGCAACACCAAGGTGGACAAG |
| | | AGAGTTGAGCCCAAATCTTGTGACAAAACTCACACATGCCCAC |
| | | CGTGCCCAGCACCTGAACTCCTGGGGGGACCGTCAGTCTTCC |
| | | TCTTCCCCCCAAAACCCAAGGACACCCTCATGATCTCCCGGAC |
| | | CCCTGAGGTCACATGCGTGGTGGTGGACGTGAGCCACGAAGA |
| | | CCCTGAGGTCAAGTTCAACTGGTACGTGGACGGCGTGGAGGT |
| | | GCATAATGCCAAGACAAAGCCGCGGGAGGAGCAGTACGCCA |
| | | GCACGTACCGGGTGGTCAGCGTCCTCACCGTCCTGCACCAGG |
| | | ACTGGCTGAATGGCAAGGAGTACAAGTGCAAGGTCTCCAACA |
| | | AAGCCCTCCCAGCCCCCATCGAGAAAACCATCTCCAAAGCCA |
| | | AAGGGCAGCCCCGAGAACCACAGGTGTACACCCTGCCCCCAT |
| | | CCCGGGAGGAGATGACCAAGAACCAGGTCAGCCTGACCTGC |
| | | CTGGTCAAAGGCTTCTATCCCAGCGACATCGCCGTGGAGTGG |
| | | GAGAGCAATGGGCAGCCGGAGAACAACTACAAGACCACGCCT |
| | | CCCGTGCTGGACTCCGACGGCTCCTTCTTCCTCTACAGCAAG |
| | | CTCACCGTGGACAAGAGCAGGTGGCAGCAGGGGAACGTCTTC |
| | | TCATGCTCCGTGATGCATGAGGCTCTGCACAACCACTACACG |
| | | CAGAAGAGCCTCTCCCTGTCTCCGGGTAAA |
| 456 | LCDR1 | SGDNIPQHSVH |
| | (Combined) | |
| 457 | LCDR2 | DDTERPS |
| | (Combined) | |
| 458 | LCDR3 | SSWDSSMDSVV |
| | (Combined) | |
| 459 | LCDR1 | SGDNIPQHSVH |
| | (Kabat) | |
| 460 | LCDR2 | DDTERPS |
| | (Kabat) | |
| 461 | LCDR3 | SSWDSSMDSVV |
| | (Kabat) | |
| 462 | LCDR1 | DNIPQHS |
| | (Chothia) | |
| 463 | LCDR2 | DDT |
| | (Chothia) | |
| 464 | LCDR3 | WDSSMDSV |
| | (Chothia) | |
| 465 | VL | SYELTQPLSVSVALGQTARITCSGDNIPQHSVHVWQQKPGQAPV |
| | | LVIYDDTERPSGIPERFSGSNSGNTATLTISRAQAGDEADYYCSS |
| | | WDSSMDSVVFGGGTKLTVL |
| 466 | VH DNA | AGCTACGAACTGACCCAGCCGCTGAGCGTGAGCGTGGCCCT |
| | | GGGCCAGACCGCGAGGATTACCTGTAGCGGCGATAACATCCC |
| | | GCAGCATTCTGTTCATTGGTACCAGCAGAAACCGGGCCAGGC |
| | | GCCGGTGCTGGTGATCTACGACGACACTGAACGTCCGAGCGG |
| | | CATCCCGGAACGTTTTAGCGGATCCAACAGCGGCAACACCGC |
| | | GACCCTGACCATTAGCAGGGCCCAGGCGGGCGACGAAGCGG |
| | | ATTATTACTGCTCTTCTTGGGACTCTTCTATGGACTCTGTTGTG |
| | | TTTGGCGGCGGCACGAAGTTAACCGTCCTA |
| 467 | Light Chain | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSWFGGGTKLTVLGQPKAAPSVTLFPPSSEELQANKAT |
| | | LVCLISDFYPGAVTVAWKADSSPVKAGVETTTPSKQSNNKYAAS |
| | | SYLSLTPEQWKSHRSYSCQVTHEGSTVEKTVAPTECS |
| 468 | Light Chain | AGCTACGAACTGACCCAGCCGCTGAGCGTGAGCGTGGCCCT |
| | DNA | GGGCCAGACCGCGAGGATTACCTGTAGCGGCGATAACATCCC |
| | | GCAGCATTCTGTTCATTGGTACCAGCAGAAACCGGGCCAGGC |
| | | GCCGGTGCTGGTGATCTACGACGACACTGAACGTCCGAGCGG |
| | | CATCCCGGAACGTTTTAGCGGATCCAACAGCGGCAACACCGC |
| | | GACCCTGACCATTAGCAGGGCCCAGGCGGGCGACGAAGCGG |
| | | ATTATTACTGCTCTTCTTGGGACTCTTCTATGGACTCTGTTGTG |
| | | TTTGGCGGCGGCACGAAGTTAACCGTCCTAGGTCAGCCCAAG |
| | | GCTGCCCCCTCGGTCACTCTGTTCCCGCCCTCCTCTGAGGAG |
| | | CTTCAAGCCAACAAGGCCACACTGGTGTGTCTCATAAGTGACT |
| | | TCTACCCGGGAGCCGTGACAGTGGCCTGGAAGGCAGATAGCA |
| | | GCCCCGTCAAGGCGGGAGTGGAGACCACCACACCCTCCAAA |
| | | CAAAGCAACAACAAGTACGCGGCCAGCAGCTATCTGAGCCTG |
| | | ACGCCTGAGCAGTGGAAGTCCCACAGAAGCTACAGCTGCCAG |
| | | GTCACGCATGAAGGGAGCACCGTGGAGAAGACAGTGGCCCC |
| | | TACAGAATGTTCA |

Antibody 019

| | | |
|---|---|---|
| 469 | HCDR1 | GGTFRDYAIS |
| | (Combined) | |
| 470 | HCDR2 | GIIPAFGTANYAQKFQG |
| | (Combined) | |
| 471 | HCDR3 | EQDPEYGFGGYPYEAMDV |
| | (Combined) | |
| 472 | HCDR1 | DYAIS |
| | (Kabat) | |
| 473 | HCDR2 | GIIPAFGTANYAQKFQG |
| | (Kabat) | |
| 474 | HCDR3 | EQDPEYGFGGYPYEAMDV |
| | (Kabat) | |
| 475 | HCDR1 | GGTFRDY |
| | (Chothia) | |
| 476 | HCDR2 | IPAFGT |
| | (Chothia) | |
| 477 | HCDR3 | EQDPEYGFGGYPYEAMDV |
| | (Chothia) | |
| 478 | VH | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQDPEYGFGGYPYEAMDVWGQGTLVTVSS |
| 479 | VH DNA | CAGGTGCAGCTGGTGCAGTCAGGCGCCGAAGTGAAGAAACC |
| | | CGGCTCTAGCGTGAAAGTCAGCTGTAAAGCTAGTGGCGGCAC |
| | | CTTTAGAGACTACGCTATTAGCTGGGTCAGACAGGCCCCAGG |
| | | TCAGGGCCTGGAGTGGATGGGCGGAATTATCCCCGCCTTCGG |
| | | CACCGCTAACTACGCTCAGAAATTTCAGGGTAGAGTGACTATC |
| | | ACCGCCGACGAGTCTACTAGCACCGCCTATATGGAACTGTCTA |
| | | GCCTGAGATCAGAGGACACCGCCGTCTACTACTGCGCTAGAG |
| | | AGCAGGACCCCGAGTACGGCTTCGGCGGCTACCCCTACGAG |
| | | GCTATGGACGTGTGGGGTCAGGGCACCCTGGTCACCGTGTCT |
| | | AGC |
| 480 | Heavy Chain | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQDPEYGFGGYPYEAMDVWGQGTLVTVSSASTK |
| | | GPSVFPLAPSSKSTSGGTAALGCLVKDYFPEPVTVSWNSGALTS |
| | | GVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISR |
| | | TPEVTCVVVDVSHEDPEVKFNWYVDGVEVHNAKTKPREEQYNS |
| | | TYRVVSVLTVLHQDWLNGKEYKCKVSNKALPAPIEKTISKAKGQP |
| | | REPQVYTLPPSREEMTKNQVSLTCLVKGFYPSDIAVEWESNGQP |
| | | ENNYKTTPPVLDSDGSFFLYSKLTVDKSRWQQGNVFSCSVMHE |
| | | ALHNHYTQKSLSLSPGK |
| 481 | Heavy Chain | CAGGTGCAGCTGGTGCAGTCAGGCGCCGAAGTGAAGAAACC |
| | DNA | CGGCTCTAGCGTGAAAGTCAGCTGTAAAGCTAGTGGCGGCAC |
| | | CTTTAGAGACTACGCTATTAGCTGGGTCAGACAGGCCCCAGG |
| | | TCAGGGCCTGGAGTGGATGGGCGGAATTATCCCCGCCTTCGG |
| | | CACCGCTAACTACGCTCAGAAATTTCAGGGTAGAGTGACTATC |
| | | ACCGCCGACGAGTCTACTAGCACCGCCTATATGGAACTGTCTA |
| | | GCCTGAGATCAGAGGACACCGCCGTCTACTACTGCGCTAGAG |
| | | AGCAGGACCCCGAGTACGGCTTCGGCGGCTACCCCTACGAG |
| | | GCTATGGACGTGTGGGGTCAGGGCACCCTGGTCACCGTGTCT |
| | | AGCGCTAGCACTAAGGGCCCAAGTGTGTTTCCCCTGGCCCCC |
| | | AGCAGCAAGTCTACTTCCGGCGGAACTGCTGCCCTGGGTTGC |
| | | CTGGTGAAGGACTACTTCCCCGAGCCCGTGACAGTGTCCTGG |
| | | AACTCTGGGGCTCTGACTTCCGGCGTGCACACCTTCCCCGCC |
| | | GTGCTGCAGAGCAGCGGCCTGTACAGCCTGAGCAGCGTGGT |
| | | GACAGTGCCCTCCAGCTCTCTGGGAACCCAGACCTATATCTG |
| | | CAACGTGAACCACAAGCCCAGCAACACCAAGGTGGACAAGAG |
| | | AGTGGAGCCCAAGAGCTGCGACAAGACCCACACCTGCCCCCC |
| | | CTGCCCAGCTCCAGAACTGCTGGGAGGGCCTTCCGTGTTCCT |
| | | GTTCCCCCCCAAGCCCAAGGACACCCTGATGATCAGCAGGAC |
| | | CCCCGAGGTGACCTGCGTGGTGGTGGACGTGTCCCACGAGG |
| | | ACCCAGAGGTGAAGTTCAACTGGTACGTGGACGGCGTGGAGG |
| | | TGCACAACGCCAAGACCAAGCCCAGAGAGGAGCAGTACAACA |
| | | GCACCTACAGGGTGGTGTCCGTGCTGACCGTGCTGCACCAGG |
| | | ACTGGCTGAACGGCAAAGAATACAAGTGCAAAGTCTCCAACAA |
| | | GGCCCTGCCAGCCCCAATCGAAAAGACAATCAGCAAGGCCAA |
| | | GGGCCAGCCACGGGAGCCCCAGGTGTACACCCTGCCCCCCA |
| | | GCCGGGAGGAGATGACCAAGAACCAGGTGTCCCTGACCTGTC |
| | | TGGTGAAGGGCTTCTACCCCAGCGATATCGCCGTGGAGTGGG |
| | | AGAGCAACGGCCAGCCCGAGAACAACTACAAGACCACCCCCC |
| | | CAGTGCTGGACAGCGACGGCAGCTTCTTCCTGTACAGCAAGC |
| | | TGACCGTGGACAAGTCCAGGTGGCAGCAGGGCAACGTGTTCA |
| | | GCTGCAGCGTGATGCACGAGGCCCTGCACAACCACTACACCC |
| | | AGAAGTCCCTGAGCCTGAGCCCCGGCAAG |
| 482 | LCDR1 | SGDNIPQHSVH |
| | (Combined) | |
| 483 | LCDR2 | DDTERPS |
| | (Combined) | |
| 484 | LCDR3 | SSWDSSMDSVV |
| | (Combined) | |
| 485 | LCDR1 | SGDNIPQHSVH |
| | (Kabat) | |
| 486 | LCDR2 | DDTERPS |
| | (Kabat) | |
| 487 | LCDR3 | SSWDSSMDSVV |
| | (Kabat) | |
| 488 | LCDR1 | DNIPQHS |
| | (Chothia) | |
| 489 | LCDR2 | DDT |
| | (Chothia) | |
| 490 | LCDR3 | WDSSMDSV |
| | (Chothia) | |
| 491 | VL | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSVVFGGGTKLTVL |
| 492 | VH DNA | AGCTACGAGCTGACTCAGCCCCTGAGCGTCAGCGTGGCCCTG |
| | | GGTCAGACCGCTAGAATCACCTGTAGCGGCGATAATATCCCT |
| | | CAGCACTCAGTGCACTGGTATCAGCAGAAGCCCGGTCAGGCC |
| | | CCCGTGCTGGTGATCTACGACGACACCGAGCGGCCTAGCGG |
| | | AATCCCCGAGCGGTTTAGCGGCTCTAATAGCGGTAACACCGC |
| | | TACCCTGACTATCTCTAGGGCTCAGGCCGGCGACGAGGCCGA |
| | | CTACTACTGCTCTAGCTGGGATAGCTCTATGGATAGCGTGGTG |
| | | TTCGGCGGAGGCACTAAGCTGACCGTGCTG |
| 493 | Light Chain | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSVVFGGGTKLTVLGQPKAAPSVTLFPPSSEELQANKAT |
| | | LVCLISDFYPGAVTVAWKADSSPVKAGVETTTPSKQSNNKYAAS |
| | | SYLSLTPEQWKSHRSYSCQVTHEGSTVEKTVAPTECS |
| 494 | Light Chain | AGCTACGAGCTGACTCAGCCCCTGAGCGTCAGCGTGGCCCTG |
| | DNA | GGTCAGACCGCTAGAATCACCTGTAGCGGCGATAATATCCCT |
| | | CAGCACTCAGTGCACTGGTATCAGCAGAAGCCCGGTCAGGCC |
| | | CCCGTGCTGGTGATCTACGACGACACCGAGCGGCCTAGCGG |
| | | AATCCCCGAGCGGTTTAGCGGCTCTAATAGCGGTAACACCGC |
| | | TACCCTGACTATCTCTAGGGCTCAGGCCGGCGACGAGGCCGA |
| | | CTACTACTGCTCTAGCTGGGATAGCTCTATGGATAGCGTGGTG |
| | | TTCGGCGGAGGCACTAAGCTGACCGTGCTGGGTCAGCCTAAG |
| | | GCTGCCCCCAGCGTGACCCTGTTCCCCCCCAGCAGCGAGGA |
| | | GCTGCAGGCCAACAAGGCCACCCTGGTGTGCCTGATCAGCGA |
| | | CTTCTACCCAGGCGCCGTGACCGTGGCCTGGAAGGCCGACA |
| | | GCAGCCCCGTGAAGGCCGGCGTGGAGACCACCACCCCCAGC |
| | | AAGCAGAGCAACAACAAGTACGCCGCCAGCAGCTACCTGAGC |
| | | CTGACCCCCGAGCAGTGGAAGAGCCACAGGTCCTACAGCTGC |
| | | CAGGTGACCCACGAGGGCAGCACCGTGGAAAAGACCGTGGC |
| | | CCCAACCGAGTGCAGC |

Antibody 020

| | | |
|---|---|---|
| 495 | HCDR1 | GGTFRDYAIS |
| | (Combined) | |
| 496 | HCDR2 | GIIPAFGTANYAQKFQG |
| | (Combined) | |
| 497 | HCDR3 | EQDPEYGFGGYPYEAMDV |
| | (Combined) | |
| 498 | HCDR1 | DYAIS |
| | (Kabat) | |
| 499 | HCDR2 | GIIPAFGTANYAQKFQG |
| | (Kabat) | |
| 500 | HCDR3 | EQDPEYGFGGYPYEAMDV |
| | (Kabat) | |
| 501 | HCDR1 | GGTFRDY |
| | (Chothia) | |
| 502 | HCDR2 | IPAFGT |
| | (Chothia) | |
| 503 | HCDR3 | EQDPEYGFGGYPYEAMDV |
| | (Chothia) | |
| 504 | VH | QVQLVQSGAEVKKPGSSVKVSCKASGGTFRDYAISWVRQAPGQ |
| | | GLEWMGGIIPAFGTANYAQKFQGRVTITADESTSTAYMELSSLRS |
| | | EDTAVYYCAREQDPEYGFGGYPYEAMDVWGQGTLVTVSS |
| 505 | VH DNA | CAGGTGCAATTGGTGCAGAGCGGTGCCGAAGTGAAAAAACCG |
| | | GGCAGCAGCGTGAAAGTTAGCTGCAAAGCATCCGGAGGGAC |
| | | GTTTCGTGACTACGCTATCTCTTGGGTGCGCCAGGCCCCGGG |
| | | CCAGGGCCTCGAGTGGATGGGCGGTATCATCCCGGCTTTCGG |
| | | CACTGCGAACTACGCCCAGAAATTTCAGGGCCGGGTGACCAT |
| | | TACCGCCGATGAAAGCACCAGCACCGCCTATATGGAACTGAG |
| | | CAGCCTGCGCAGCGAAGATACGGCCGTGTATTATTGCGCGCG |
| | | TGAACAGGACCCGGAATACGGTTTCGGTGGTTACCCGTATGA |
| | | AGCTATGGATGTTTGGGGCCAAGGCACCCTGGTGACTGTTAG |
| | | CTCA |
| 506 | Heavy Chain | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQDPEYGFGGYPYEAMDVWGQGTLVTVSSASTK |
| | | GPSVFPLAPSSKSTSGGTAALGCLVKDYFPEPVTVSWNSGALTS |
| | | GVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISR |
| | | TPEVTCVVVDVSHEDPEVKFNWYVDGVEVHNAKTKPREEQYAS |
| | | TYRVVSVLTVLHQDWLNGKEYKCKVSNKALPAPIEKTISKAKGQP |
| | | REPQVYTLPPSREEMTKNQVSLTCLVKGFYPSDIAVEWESNGQP |
| | | ENNYKTTPPVLDSDGSFFLYSKLTVQKSRWQQGNVFSCSVMHE |
| | | ALHNHYTQKSLSLSPGK |
| 507 | Heavy Chain | CAGGTGCAATTGGTGCAGAGCGGTGCCGAAGTGAAAAAACCG |
| | DNA | GGCAGCAGCGTGAAAGTTAGCTGCAAAGCATCCGGAGGGAC |
| | | GTTTCGTGACTACGCTATCTCTTGGGTGCGCCAGGCCCCGGG |
| | | CCAGGGCCTCGAGTGGATGGGCGGTATCATCCCGGCTTTCGG |
| | | CACTGCGAACTACGCCCAGAAATTTCAGGGCCGGGTGACCAT |
| | | TACCGCCGATGAAAGCACCAGCACCGCCTATATGGAACTGAG |
| | | CAGCCTGCGCAGCGAAGATACGGCCGTGTATTATTGCGCGCG |
| | | TGAACAGGACCCGGAATACGGTTTCGGTGGTTACCCGTATGA |
| | | AGCTATGGATGTTTGGGGCCAAGGCACCCTGGTGACTGTTAG |
| | | CTCAGCCTCCACCAAGGGTCCATCGGTCTTCCCCCTGGCACC |
| | | CTCCTCCAAGAGCACCTCTGGGGGCACAGCGGCCCTGGGCT |
| | | GCCTGGTCAAGGACTACTTCCCCGAACCGGTGACGGTGTCGT |
| | | GGAACTCAGGCGCCCTGACCAGCGGCGTGCACACCTTCCCG |
| | | GCTGTCCTACAGTCCTCAGGACTCTACTCCCTCAGCAGCGTG |
| | | GTGACCGTGCCCTCCAGCAGCTTGGGCACCCAGACCTACATC |
| | | TGCAACGTGAATCACAAGCCCAGCAACACCAAGGTGGACAAG |
| | | AGAGTTGAGCCCAAATCTTGTGACAAAACTCACACATGCCCAC |
| | | CGTGCCCAGCACCTGAACTCCTGGGGGGACCGTCAGTCTTCC |
| | | TCTTCCCCCCAAAACCCAAGGACACCCTCATGATCTCCCGGAC |
| | | CCCTGAGGTCACATGCGTGGTGGTGGACGTGAGCCACGAAGA |
| | | CCCTGAGGTCAAGTTCAACTGGTACGTGGACGGCGTGGAGGT |
| | | GCATAATGCCAAGACAAAGCCGCGGGAGGAGCAGTACGCCA |
| | | GCACGTACCGGGTGGTCAGCGTCCTCACCGTCCTGCACCAGG |
| | | ACTGGCTGAATGGCAAGGAGTACAAGTGCAAGGTCTCCAACA |
| | | AAGCCCTCCCAGCCCCCATCGAGAAAACCATCTCCAAAGCCA |
| | | AAGGGCAGCCCCGAGAACCACAGGTGTACACCCTGCCCCCAT |
| | | CCCGGGAGGAGATGACCAAGAACCAGGTCAGCCTGACCTGC |
| | | CTGGTCAAAGGCTTCTATCCCAGCGACATCGCCGTGGAGTGG |
| | | GAGAGCAATGGGCAGCCGGAGAACAACTACAAGACCACGCCT |
| | | CCCGTGCTGGACTCCGACGGCTCCTTCTTCCTCTACAGCAAG |
| | | CTCACCGTGGACAAGAGCAGGTGGCAGCAGGGGAACGTCTTC |
| | | TCATGCTCCGTGATGCATGAGGCTCTGCACAACCACTACACG |
| | | CAGAAGAGCCTCTCCCTGTCTCCGGGTAAA |
| 508 | LCDR1 | SGDNIPQHSVH |
| | (Combined) | |
| 509 | LCDR2 | DDTERPS |
| | (Combined) | |
| 510 | LCDR3 | SSWDSSMDSVV |
| | (Combined) | |
| 511 | LCDR1 | SGDNIPQHSVH |
| | (Kabat) | |
| 512 | LCDR2 | DDTERPS |
| | (Kabat) | |
| 513 | LCDR3 | SSWDSSMDSVV |
| | (Kabat) | |
| 514 | LCDR1 | DNIPQHS |
| | (Chothia) | |
| 515 | LCDR2 | DDT |
| | (Chothia) | |
| 516 | LCDR3 | WDSSMDSV |
| | (Chothia) | |
| 517 | VL | SYELTQPLSVSVALGQTARITCSGDNIPQHSVHWYQQKPGQAPV |
| | | LVIYQDTERPSGIPERFSGSNSGNTATLTISRAQAGDEADYYCSS |
| | | WDSSMDSVVFGGGTKLTVL |
| 518 | VH DNA | AGCTACGAACTGACCCAGCCGCTGAGCGTGAGCGTGGCCCT |
| | | GGGCCAGACCGCGAGGATTACCTGTAGCGGCGATAACATCCC |
| | | GCAGCATTCTGTTCATTGGTACCAGCAGAAACCGGGCCAGGC |
| | | GCCGGTGCTGGTGATCTACGACGACACTGAACGTCCGAGCGG |
| | | CATCCCGGAACGTTTTAGCGGATCCAACAGCGGCAACACCGC |
| | | GACCCTGACCATTAGCAGGGCCCAGGCGGGCGACGAAGCGG |
| | | ATTATTACTGCTCTTCTTGGGACTCTTCTATGGACTCTGTTGTG |
| | | TTTGGCGGCGGCACGAAGTTAACCGTCCTA |
| 519 | Light Chain | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSWFGGGTKLTVLGQPKAAPSVTLFPPSSEELQANKAT |
| | | LVCLISDFYPGAVTVAWKADSSPVKAGVETTTPSKQSNNKYAAS |
| | | SYLSLTPEGWKSHRSYSCQVTHEGSTVEKTVAPTECS |
| 520 | Light Chain | AGCTACGAACTGACCCAGCCGCTGAGCGTGAGCGTGGCCCT |
| | DNA | GGGCCAGACCGCGAGGATTACCTGTAGCGGCGATAACATCCC |
| | | GCAGCATTCTGTTCATTGGTACCAGCAGAAACCGGGCCAGGC |
| | | GCCGGTGCTGGTGATCTACGACGACACTGAACGTCCGAGCGG |
| | | CATCCCGGAACGTTTTAGCGGATCCAACAGCGGCAACACCGC |
| | | GACCCTGACCATTAGCAGGGCCCAGGCGGGCGACGAAGCGG |
| | | ATTATTACTGCTCTTCTTGGGACTCTTCTATGGACTCTGTTGTG |
| | | TTTGGCGGCGGCACGAAGTTAACCGTCCTAGGTCAGCCCAAG |
| | | GCTGCCCCCTCGGTCACTCTGTTCCCGCCCTCCTCTGAGGAG |
| | | CTTCAAGCCAACAAGGCCACACTGGTGTGTCTCATAAGTGACT |
| | | TCTACCCGGGAGCCGTGACAGTGGCCTGGAAGGCAGATAGCA |
| | | GCCCCGTCAAGGCGGGAGTGGAGACCACCACACCCTCCAAA |
| | | CAAAGCAACAACAAGTACGCGGCCAGCAGCTATCTGAGCCTG |
| | | ACGCCTGAGCAGTGGAAGTCCCACAGAAGCTACAGCTGCCAG |
| | | GTCACGCATGAAGGGAGCACCGTGGAGAAGACAGTGGCCCC |
| | | TACAGAATGTTCA |

Antibody 021

| | | |
|---|---|---|
| 521 | HCDR1 | GGTFRDYAIS |
| | (Combined) | |
| 522 | HCDR2 | GIIPAFGTANYAQKFQG |
| | (Combined) | |
| 523 | HCDR3 | EQDPEYGYGGFPVEAMDV |
| | (Combined) | |
| 524 | HCDR1 | DYAIS |
| | (Kabat) | |
| 525 | HCDR2 | GIIPAFGTANYAQKFQG |
| | (Kabat) | |
| 526 | HCDR3 | EQDPEYGYGGFPYEAMDV |
| | (Kabat) | |
| 527 | HCDR1 | GGTFRDY |
| | (Chothia) | |
| 528 | HCDR2 | IPAFGT |
| | (Chothia) | |
| 529 | HCDR3 | EQDPEYGYGGFPYEAMDV |
| | (Chothia) | |
| 530 | VH | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQDPEYGYGGFPYEAMDVWGQGTLVTVSS |
| 531 | VH DNA | CAGGTGCAATTGGTGCAGAGCGGTGCCGAAGTGAAAAAACCG |
| | | GGCAGCAGCGTGAAAGTTAGCTGCAAAGCATCCGGAGGGAC |
| | | GTTTCGTGACTACGCTATCTCTTGGGTGCGCCAGGCCCCGGG |
| | | CCAGGGCCTCGAGTGGATGGGCGGTATCATCCCGGCTTTCGG |
| | | CACTGCGAACTACGCCCAGAAATTTCAGGGCCGGGTGACCAT |
| | | TACCGCCGATGAAAGCACCAGCACCGCCTATATGGAACTGAG |
| | | CAGCCTGCGCAGCGAAGATACGGCCGTGTATTATTGCGCGCG |
| | | TGAACAGGACCCGGAATACGGTTACGGTGGTTTCCCGTATGA |
| | | AGCTATGGATGTTTGGGGCCAAGGCACCCTGGTGACTGTTAG |
| | | CTCA |
| 532 | Heavy Chain | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQDPEYGYGGFPYEAMDVWGQGTLVTVSSASTK |
| | | GPSVFPLAPSSKSTSGGTAALGCLVKDYFPEPVTVSWNSGALTS |
| | | GVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISR |
| | | TPEVTCVVVDVSHEDPEVKFNWYVDGVEVHNAKTKPREEQYAS |
| | | TYRVVSVLTVLHQDWLNGKEYKCKVSNKALPAPIEKTISKAKGQP |
| | | REPQVYTLPPSREEMTKNQVSLTCLVKGFYPSDIAVEWESNGQP |
| | | ENNYKTTPPVLDSDGSFFLYSKLTVDKSRWQQGNVFSCSVMHE |
| | | ALHNHYTQKSLSLSPGK |
| 533 | Heavy Chain | CAGGTGCAATTGGTGCAGAGCGGTGCCGAAGTGAAAAAACCG |
| | DNA | GGCAGCAGCGTGAAAGTTAGCTGCAAAGCATCCGGAGGGAC |
| | | GTTTCGTGACTACGCTATCTCTTGGGTGCGCCAGGCCCCGGG |
| | | CCAGGGCCTCGAGTGGATGGGCGGTATCATCCCGGCTTTCGG |
| | | CACTGCGAACTACGCCCAGAAATTTCAGGGCCGGGTGACCAT |
| | | TACCGCCGATGAAAGCACCAGCACCGCCTATATGGAACTGAG |
| | | CAGCCTGCGCAGCGAAGATACGGCCGTGTATTATTGCGCGCG |
| | | TGAACAGGACCCGGAATACGGTTACGGTGGTTTCCCGTATGA |
| | | AGCTATGGATGTTTGGGGCCAAGGCACCCTGGTGACTGTTAG |
| | | CTCAGCCTCCACCAAGGGTCCATCGGTCTTCCCCCTGGCACC |
| | | CTCCTCCAAGAGCACCTCTGGGGGCACAGCGGCCCTGGGCT |
| | | GCCTGGTCAAGGACTACTTCCCCGAACCGGTGACGGTGTCGT |
| | | GGAACTCAGGCGCCCTGACCAGCGGCGTGCACACCTTCCCG |
| | | GCTGTCCTACAGTCCTCAGGACTCTACTCCCTCAGCAGCGTG |
| | | GTGACCGTGCCCTCCAGCAGCTTGGGCACCCAGACCTACATC |
| | | TGCAACGTGAATCACAAGCCCAGCAACACCAAGGTGGACAAG |
| | | AGAGTTGAGCCCAAATCTTGTGACAAAACTCACACATGCCCAC |
| | | CGTGCCCAGCACCTGAACTCCTGGGGGGACCGTCAGTCTTCC |
| | | TCTTCCCCCCAAAACCCAAGGACACCCTCATGATCTCCCGGAC |
| | | CCCTGAGGTCACATGCGTGGTGGTGGACGTGAGCCACGAAGA |
| | | CCCTGAGGTCAAGTTCAACTGGTACGTGGACGGCGTGGAGGT |
| | | GCATAATGCCAAGACAAAGCCGCGGGAGGAGCAGTACGCCA |
| | | GCACGTACCGGGTGGTCAGCGTCCTCACCGTCCTGCACCAGG |
| | | ACTGGCTGAATGGCAAGGAGTACAAGTGCAAGGTCTCCAACA |
| | | AAGCCCTCCCAGCCCCCATCGAGAAAACCATCTCCAAAGCCA |
| | | AAGGGCAGCCCCGAGAACCACAGGTGTACACCCTGCCCCCAT |
| | | CCCGGGAGGAGATGACCAAGAACCAGGTCAGCCTGACCTGC |
| | | CTGGTCAAAGGCTTCTATCCCAGCGACATCGCCGTGGAGTGG |
| | | GAGAGCAATGGGCAGCCGGAGAACAACTACAAGACCACGCCT |
| | | CCCGTGCTGGACTCCGACGGCTCCTTCTTCCTCTACAGCAAG |
| | | CTCACCGTGGACAAGAGCAGGTGGCAGCAGGGGAACGTCTTC |
| | | TCATGCTCCGTGATGCATGAGGCTCTGCACAACCACTACACG |
| | | CAGAAGAGCCTCTCCCTGTCTCCGGGTAAA |
| 534 | LCDR1 | SGDNIPQHSVH |
| | (Combined) | |
| 535 | LCDR2 | DDTERPS |
| | (Combined) | |
| 536 | LCDR3 | SSWDSSMDSVV |
| | (Combined) | |
| 537 | LCDR1 | SGDNIPQHSVH |
| | (Kabat) | |
| 538 | LCDR2 | DDTERPS |
| | (Kabat) | |
| 539 | LCDR3 | SSWDSSMDSVV |
| | (Kabat) | |
| 540 | LCDR1 | DNIPQHS |
| | (Chothia) | |
| 541 | LCDR2 | DDT |
| | (Chothia) | |
| 542 | LCDR3 | WDSSMDSV |
| | (Chothia) | |
| 543 | VL | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSVVFGGGTKLTVL |
| 544 | VH DNA | AGCTACGAACTGACCCAGCCGCTGAGCGTGAGCGTGGCCCT |
| | | GGGCCAGACCGCGAGGATTACCTGTAGCGGCGATAACATCCC |
| | | GCAGCATTCTGTTCATTGGTACCAGCAGAAACCGGGCCAGGC |
| | | GCCGGTGCTGGTGATCTACGACGACACTGAACGTCCGAGCGG |
| | | CATCCCGGAACGTTTTAGCGGATCCAACAGCGGCAACACCGC |
| | | GACCCTGACCATTAGCAGGGCCCAGGCGGGCGACGAAGCGG |
| | | ATTATTACTGCTCTTCTTGGGACTCTTCTATGGACTCTGTTGTG |
| | | TTTGGCGGCGGCACGAAGTTAACCGTCCTA |
| 545 | Light Chain | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSVVFGGGTKLTVLGQPKAAPSVTLFPPSSEELQANKAT |
| | | LVCLISDFYPGAVTVAWKADSSPVKAGVETTTPSKQSNNKYAAS |
| | | SYLSLTPEQWKSHRSYSCQVTHEGSTVEKTVAPTECS |
| 546 | Light Chain | AGCTACGAACTGACCCAGCCGCTGAGCGTGAGCGTGGCCCT |
| | DNA | GGGCCAGACCGCGAGGATTACCTGTAGCGGCGATAACATCCC |
| | | GCAGCATTCTGTTCATTGGTACCAGCAGAAACCGGGCCAGGC |
| | | GCCGGTGCTGGTGATCTACGACGACACTGAACGTCCGAGCGG |
| | | CATCCCGGAACGTTTTAGCGGATCCAACAGCGGCAACACCGC |
| | | GACCCTGACCATTAGCAGGGCCCAGGCGGGCGACGAAGCGG |
| | | ATTATTACTGCTCTTCTTGGGACTCTTCTATGGACTCTGTTGTG |
| | | TTTGGCGGCGGCACGAAGTTAACCGTCCTAGGTCAGCCCAAG |
| | | GCTGCCCCCTCGGTCACTCTGTTCCCGCCCTCCTCTGAGGAG |
| | | CTTCAAGCCAACAAGGCCACACTGGTGTGTCTCATAAGTGACT |
| | | TCTACCCGGGAGCCGTGACAGTGGCCTGGAAGGCAGATAGCA |
| | | GCCCCGTCAAGGCGGGAGTGGAGACCACCACACCCTCCAAA |
| | | CAAAGCAACAACAAGTACGCGGCCAGCAGCTATCTGAGCCTG |
| | | ACGCCTGAGCAGTGGAAGTCCCACAGAAGCTACAGCTGCCAG |
| | | GTCACGCATGAAGGGAGCACCGTGGAGAAGACAGTGGCCCC |
| | | TACAGAATGTTCA |

Antibody 022

| | | |
|---|---|---|
| 547 | HCDR1 | GGTFRDYAIS |
| | (Combined) | |
| 548 | HCDR2 | GIIPAFGTANYAQKFQG |
| | (Combined) | |
| 549 | HCDR3 | EQDPEYGYGGYPFEAMDV |
| | (Combined) | |
| 550 | HCDR1 | DYAIS |
| | (Kabat) | |
| 551 | HCDR2 | GIIPAFGTANYAQKFQG |
| | (Kabat) | |
| 552 | HCDR3 | EQDPEYGYGGYPFEAMDV |
| | (Kabat) | |
| 553 | HCDR1 | GGTFRDY |
| | (Chothia) | |
| 554 | HCDR2 | IPAFGT |
| | (Chothia) | |
| 555 | HCDR3 | EQDPEYGYGGYPFEAMDV |
| | (Chothia) | |
| 556 | VH | QVQLVQSGAEVKKPGSSVKVSCKASGGTFRDYAISWVRQAPGQ |
| | | GLEWMGGIIPAFGTANYAQKFQGRVTITADESTSTAYMELSSLRS |
| | | EDTAVYYCAREQDPEYGYGGYPFEAMDVWGQGTLVTVSS |
| 557 | VH DNA | CAGGTGCAATTGGTGCAGAGCGGTGCCGAAGTGAAAAAACCG |
| | | GGCAGCAGCGTGAAAGTTAGCTGCAAAGCATCCGGAGGGAC |
| | | GTTTCGTGACTACGCTATCTCTTGGGTGCGCCAGGCCCCGGG |
| | | CCAGGGCCTCGAGTGGATGGGCGGTATCATCCCGGCTTTCGG |
| | | CACTGCGAACTACGCCCAGAAATTTCAGGGCCGGGTGACCAT |
| | | TACCGCCGATGAAAGCACCAGCACCGCCTATATGGAACTGAG |
| | | CAGCCTGCGCAGCGAAGATACGGCCGTGTATTATTGCGCGCG |
| | | TGAACAGGACCCGGAATACGGTTACGGTGGTTACCCGTTCGA |
| | | AGCTATGGATGTTTGGGGCCAAGGCACCCTGGTGACTGTTAG |
| | | CTCA |
| 558 | Heavy Chain | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQDPEYGYGGYPFEAMDVWGQGTLVTVSSASTK |
| | | GPSVFPLAPSSKSTSGGTAALGCLVKDYFPEPVTVSWNSGALTS |
| | | GVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISR |
| | | TPEVTCVVVDVSHEDPEVKFNWYVDGVEVHNAKTKPREEQYAS |
| | | TYRVVSVLTVLHQDWLNGKEYKCKVSNKALPAPIEKTISKAKGQP |
| | | REPQVYTLPPSREEMTKNQVSLTCLVKGFYPSDIAVEWESNGQP |
| | | ENNYKTTPPVLDSDGSFFLYSKLTVQKSRWQQGNVFSCSVMHE |
| | | ALHNHYTQKSLSLSPGK |
| 559 | Heavy Chain | CAGGTGCAATTGGTGCAGAGCGGTGCCGAAGTGAAAAAACCG |
| | DNA | GGCAGCAGCGTGAAAGTTAGCTGCAAAGCATCCGGAGGGAC |
| | | GTTTCGTGACTACGCTATCTCTTGGGTGCGCCAGGCCCCGGG |
| | | CCAGGGCCTCGAGTGGATGGGCGGTATCATCCCGGCTTTCGG |
| | | CACTGCGAACTACGCCCAGAAATTTCAGGGCCGGGTGACCAT |
| | | TACCGCCGATGAAAGCACCAGCACCGCCTATATGGAACTGAG |
| | | CAGCCTGCGCAGCGAAGATACGGCCGTGTATTATTGCGCGCG |
| | | TGAACAGGACCCGGAATACGGTTACGGTGGTTACCCGTTCGA |
| | | AGCTATGGATGTTTGGGGCCAAGGCACCCTGGTGACTGTTAG |
| | | CTCAGCCTCCACCAAGGGTCCATCGGTCTTCCCCCTGGCACC |
| | | CTCCTCCAAGAGCACCTCTGGGGGCACAGCGGCCCTGGGCT |
| | | GCCTGGTCAAGGACTACTTCCCCGAACCGGTGACGGTGTCGT |
| | | GGAACTCAGGCGCCCTGACCAGCGGCGTGCACACCTTCCCG |
| | | GCTGTCCTACAGTCCTCAGGACTCTACTCCCTCAGCAGCGTG |
| | | GTGACCGTGCCCTCCAGCAGCTTGGGCACCCAGACCTACATC |
| | | TGCAACGTGAATCACAAGCCCAGCAACACCAAGGTGGACAAG |
| | | AGAGTTGAGCCCAAATCTTGTGACAAAACTCACACATGCCCAC |
| | | CGTGCCCAGCACCTGAACTCCTGGGGGGACCGTCAGTCTTCC |
| | | TCTTCCCCCCAAAACCCAAGGACACCCTCATGATCTCCCGGAC |
| | | CCCTGAGGTCACATGCGTGGTGGTGGACGTGAGCCACGAAGA |
| | | CCCTGAGGTCAAGTTCAACTGGTACGTGGACGGCGTGGAGGT |
| | | GCATAATGCCAAGACAAAGCCGCGGGAGGAGCAGTACGCCA |
| | | GCACGTACCGGGTGGTCAGCGTCCTCACCGTCCTGCACCAGG |
| | | ACTGGCTGAATGGCAAGGAGTACAAGTGCAAGGTCTCCAACA |
| | | AAGCCCTCCCAGCCCCCATCGAGAAAACCATCTCCAAAGCCA |
| | | AAGGGCAGCCCCGAGAACCACAGGTGTACACCCTGCCCCCAT |
| | | CCCGGGAGGAGATGACCAAGAACCAGGTCAGCCTGACCTGC |
| | | CTGGTCAAAGGCTTCTATCCCAGCGACATCGCCGTGGAGTGG |
| | | GAGAGCAATGGGCAGCCGGAGAACAACTACAAGACCACGCCT |
| | | CCCGTGCTGGACTCCGACGGCTCCTTCTTCCTCTACAGCAAG |
| | | CTCACCGTGGACAAGAGCAGGTGGCAGCAGGGGAACGTCTTC |
| | | TCATGCTCCGTGATGCATGAGGCTCTGCACAACCACTACACG |
| | | CAGAAGAGCCTCTCCCTGTCTCCGGGTAAA |
| 560 | LCDR1 | SGDNIPQHSVH |
| | (Combined) | |
| 561 | LCDR2 | DDTERPS |
| | (Combined) | |
| 562 | LCDR3 | SSWDSSMDSVV |
| | (Combined) | |
| 563 | LCDR1 | SGDNIPQHSVH |
| | (Kabat) | |
| 564 | LCDR2 | DDTERPS |
| | (Kabat) | |
| 565 | LCDR3 | SSWDSSMDSVV |
| | (Kabat) | |
| 566 | LCDR1 | DNIPQHS |
| | (Chothia) | |
| 567 | LCDR2 | DDT |
| | (Chothia) | |
| 568 | LCDR3 | WDSSMDSV |
| | (Chothia) | |
| 569 | VL | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSVVFGGGTKLTVL |
| 570 | VH DNA | AGCTACGAACTGACCCAGCCGCTGAGCGTGAGCGTGGCCCT |
| | | GGGCCAGACCGCGAGGATTACCTGTAGCGGCGATAACATCCC |
| | | GCAGCATTCTGTTCATTGGTACCAGCAGAAACCGGGCCAGGC |
| | | GCCGGTGCTGGTGATCTACGACGACACTGAACGTCCGAGCGG |
| | | CATCCCGGAACGTTTTAGCGGATCCAACAGCGGCAACACCGC |
| | | GACCCTGACCATTAGCAGGGCCCAGGCGGGCGACGAAGCGG |
| | | ATTATTACTGCTCTTCTTGGGACTCTTCTATGGACTCTGTTGTG |
| | | TTTGGCGGCGGCACGAAGTTAACCGTCCTA |
| 571 | Light Chain | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSVVFGGGTKLTVLGQPKAAPSVTLFPPSSEELQANKAT |
| | | LVCLISDFYPGAVTVAWKADSSPVKAGVETTTPSKQSNNKYAAS |
| | | SYLSLTPEQWKSHRSYSCQVTHEGSTVEKTVAPTECS |
| 572 | Light Chain | AGCTACGAACTGACCCAGCCGCTGAGCGTGAGCGTGGCCCT |
| | DNA | GGGCCAGACCGCGAGGATTACCTGTAGCGGCGATAACATCCC |
| | | GCAGCATTCTGTTCATTGGTACCAGCAGAAACCGGGCCAGGC |
| | | GCCGGTGCTGGTGATCTACGACGACACTGAACGTCCGAGCGG |
| | | CATCCCGGAACGTTTTAGCGGATCCAACAGCGGCAACACCGC |
| | | GACCCTGACCATTAGCAGGGCCCAGGCGGGCGACGAAGCGG |
| | | ATTATTACTGCTCTTCTTGGGACTCTTCTATGGACTCTGTTGTG |
| | | TTTGGCGGCGGCACGAAGTTAACCGTCCTAGGTCAGCCCAAG |
| | | GCTGCCCCCTCGGTCACTCTGTTCCCGCCCTCCTCTGAGGAG |
| | | CTTCAAGCCAACAAGGCCACACTGGTGTGTCTCATAAGTGACT |
| | | TCTACCCGGGAGCCGTGACAGTGGCCTGGAAGGCAGATAGCA |
| | | GCCCCGTCAAGGCGGGAGTGGAGACCACCACACCCTCCAAA |
| | | CAAAGCAACAACAAGTACGCGGCCAGCAGCTATCTGAGCCTG |
| | | ACGCCTGAGCAGTGGAAGTCCCACAGAAGCTACAGCTGCCAG |
| | | GTCACGCATGAAGGGAGCACCGTGGAGAAGACAGTGGCCCC |
| | | TACAGAATGTTCA |

Antibody 023

| | | |
|---|---|---|
| 573 | HCDR1 | GGTFRDYAIS |
| | (Combined) | |
| 574 | HCDR2 | GIIPAFGTANYAQKFQG |
| | (Combined) | |
| 575 | HCDR3 | EQDPSYGYGGYPYEAMDV |
| | (Combined) | |
| 576 | HCDR1 | DYAIS |
| | (Kabat) | |
| 577 | HCDR2 | GIIPAFGTANYAQKFQG |
| | (Kabat) | |
| 578 | HCDR3 | EQDPSYGYGGYPYEAMDV |
| | (Kabat) | |
| 579 | HCDR1 | GGTFRDY |
| | (Chothia) | |
| 580 | HCDR2 | IPAFGT |
| | (Chothia) | |
| 581 | HCDR3 | EQDPSYGYGGYPYEAMDV |
| | (Chothia) | |
| 582 | VH | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQDPSYGYGGYPYEAMDVWGQGTLVTVSS |
| 583 | VH DNA | CAGGTGCAGCTGGTGCAGTCAGGCGCCGAAGTGAAGAAACC |
| | | CGGCTCTAGCGTGAAAGTCAGCTGTAAAGCTAGTGGCGGCAC |
| | | CTTTAGAGACTACGCTATTAGCTGGGTCAGACAGGCCCCAGG |
| | | TCAGGGCCTGGAGTGGATGGGCGGAATTATCCCCGCCTTCGG |
| | | CACCGCTAACTACGCTCAGAAATTTCAGGGTAGAGTGACTATC |
| | | ACCGCCGACGAGTCTACTAGCACCGCCTATATGGAACTGTCTA |
| | | GCCTGAGATCAGAGGACACCGCCGTCTACTACTGCGCTAGAG |
| | | AGCAGGACCCCTCCTACGGCTACGGCGGCTACCCCTACGAG |
| | | GCTATGGACGTGTGGGGTCAGGGCACCCTGGTCACCGTGTCT |
| | | AGC |
| 584 | Heavy Chain | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQDPSYGYGGYPYEAMDVWGQGTLVTVSSASTK |
| | | GPSVFPLAPSSKSTSGGTAALGCLVKDYFPEPVTVSWNSGALTS |
| | | GVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISR |
| | | TPEVTCVVVDVSHEDPEVKFNWYVDGVEVHNAKTKPREEQYNS |
| | | TYRVVSVLTVLHQDWLNGKEYKCKVSNKALPAPIEKTISKAKGQP |
| | | REPQVYTLPPSREEMTKNQVSLTCLVKGFYPSDIAVEWESNGQP |
| | | ENNYKTTPPVLDSDGSFFLYSKLTVDKSRWQQGNVFSCSVMHE |
| | | ALHNHYTQKSLSLSPGK |
| 585 | Heavy Chain | CAGGTGCAGCTGGTGCAGTCAGGCGCCGAAGTGAAGAAACC |
| | DNA | CGGCTCTAGCGTGAAAGTCAGCTGTAAAGCTAGTGGCGGCAC |
| | | CTTTAGAGACTACGCTATTAGCTGGGTCAGACAGGCCCCAGG |
| | | TCAGGGCCTGGAGTGGATGGGCGGAATTATCCCCGCCTTCGG |
| | | CACCGCTAACTACGCTCAGAAATTTCAGGGTAGAGTGACTATC |
| | | ACCGCCGACGAGTCTACTAGCACCGCCTATATGGAACTGTCTA |
| | | GCCTGAGATCAGAGGACACCGCCGTCTACTACTGCGCTAGAG |
| | | AGCAGGACCCCTCCTACGGCTACGGCGGCTACCCCTACGAG |
| | | GCTATGGACGTGTGGGGTCAGGGCACCCTGGTCACCGTGTCT |
| | | AGCGCTAGCACTAAGGGCCCAAGTGTGTTTCCCCTGGCCCCC |
| | | AGCAGCAAGTCTACTTCCGGCGGAACTGCTGCCCTGGGTTGC |
| | | CTGGTGAAGGACTACTTCCCCGAGCCCGTGACAGTGTCCTGG |
| | | AACTCTGGGGCTCTGACTTCCGGCGTGCACACCTTCCCCGCC |
| | | GTGCTGCAGAGCAGCGGCCTGTACAGCCTGAGCAGCGTGGT |
| | | GACAGTGCCCTCCAGCTCTCTGGGAACCCAGACCTATATCTG |
| | | CAACGTGAACCACAAGCCCAGCAACACCAAGGTGGACAAGAG |
| | | AGTGGAGCCCAAGAGCTGCGACAAGACCCACACCTGCCCCCC |
| | | CTGCCCAGCTCCAGAACTGCTGGGAGGGCCTTCCGTGTTCCT |
| | | GTTCCCCCCCAAGCCCAAGGACACCCTGATGATCAGCAGGAC |
| | | CCCCGAGGTGACCTGCGTGGTGGTGGACGTGTCCCACGAGG |
| | | ACCCAGAGGTGAAGTTCAACTGGTACGTGGACGGCGTGGAGG |
| | | TGCACAACGCCAAGACCAAGCCCAGAGAGGAGCAGTACAACA |
| | | GCACCTACAGGGTGGTGTCCGTGCTGACCGTGCTGCACCAGG |
| | | ACTGGCTGAACGGCAAAGAATACAAGTGCAAAGTCTCCAACAA |
| | | GGCCCTGCCAGCCCCAATCGAAAAGACAATCAGCAAGGCCAA |
| | | GGGCCAGCCACGGGAGCCCCAGGTGTACACCCTGCCCCCCA |
| | | GCCGGGAGGAGATGACCAAGAACCAGGTGTCCCTGACCTGTC |
| | | TGGTGAAGGGCTTCTACCCCAGCGATATCGCCGTGGAGTGGG |
| | | AGAGCAACGGCCAGCCCGAGAACAACTACAAGACCACCCCCC |
| | | CAGTGCTGGACAGCGACGGCAGCTTCTTCCTGTACAGCAAGC |
| | | TGACCGTGGACAAGTCCAGGTGGCAGCAGGGCAACGTGTTCA |
| | | GCTGCAGCGTGATGCACGAGGCCCTGCACAACCACTACACCC |
| | | AGAAGTCCCTGAGCCTGAGCCCCGGCAAG |
| 586 | LCDR1 | SGDNIPQHSVH |
| | (Combined) | |
| 587 | LCDR2 | DDTERPS |
| | (Combined) | |
| 588 | LCDR3 | SSWDSSMDSVV |
| | (Combined) | |
| 589 | LCDR1 | SGDNIPQHSVH |
| | (Kabat) | |
| 590 | LCDR2 | DDTERPS |
| | (Kabat) | |
| 591 | LCDR3 | SSWDSSMDSVV |
| | (Kabat) | |
| 592 | LCDR1 | DNIPQHS |
| | (Chothia) | |
| 593 | LCDR2 | DDT |
| | (Chothia) | |
| 594 | LCDR3 | WDSSMDSV |
| | (Chothia) | |
| 595 | VL | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSWFGGGTKLTVL |
| 596 | VH DNA | AGCTACGAGCTGACTCAGCCCCTGAGCGTCAGCGTGGCCCTG |
| | | GGTCAGACCGCTAGAATCACCTGTAGCGGCGATAATATCCCT |
| | | CAGCACTCAGTGCACTGGTATCAGCAGAAGCCCGGTCAGGCC |
| | | CCCGTGCTGGTGATCTACGACGACACCGAGCGGCCTAGCGG |
| | | AATCCCCGAGCGGTTTAGCGGCTCTAATAGCGGTAACACCGC |
| | | TACCCTGACTATCTCTAGGGCTCAGGCCGGCGACGAGGCCGA |
| | | CTACTACTGCTCTAGCTGGGATAGCTCTATGGATAGCGTGGTG |
| | | TTCGGCGGAGGCACTAAGCTGACCGTGCTG |
| 597 | Light Chain | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSVVFGGGTKLTVLGQPKAAPSVTLFPPSSEELQANKAT |
| | | LVCLISDFYPGAVTVAWKADSSPVKAGVETTTPSKQSNNKYAAS |
| | | SYLSLTPEQWKSHRSYSCQVTHEGSTVEKTVAPTECS |
| 598 | Light Chain | AGCTACGAGCTGACTCAGCCCCTGAGCGTCAGCGTGGCCCTG |
| | DNA | GGTCAGACCGCTAGAATCACCTGTAGCGGCGATAATATCCCT |
| | | CAGCACTCAGTGCACTGGTATCAGCAGAAGCCCGGTCAGGCC |
| | | CCCGTGCTGGTGATCTACGACGACACCGAGCGGCCTAGCGG |
| | | AATCCCCGAGCGGTTTAGCGGCTCTAATAGCGGTAACACCGC |
| | | TACCCTGACTATCTCTAGGGCTCAGGCCGGCGACGAGGCCGA |
| | | CTACTACTGCTCTAGCTGGGATAGCTCTATGGATAGCGTGGTG |
| | | TTCGGCGGAGGCACTAAGCTGACCGTGCTGGGTCAGCCTAAG |
| | | GCTGCCCCCAGCGTGACCCTGTTCCCCCCCAGCAGCGAGGA |
| | | GCTGCAGGCCAACAAGGCCACCCTGGTGTGCCTGATCAGCGA |
| | | CTTCTACCCAGGCGCCGTGACCGTGGCCTGGAAGGCCGACA |
| | | GCAGCCCCGTGAAGGCCGGCGTGGAGACCACCACCCCCAGC |
| | | AAGCAGAGCAACAACAAGTACGCCGCCAGCAGCTACCTGAGC |
| | | CTGACCCCCGAGCAGTGGAAGAGCCACAGGTCCTACAGCTGC |
| | | CAGGTGACCCACGAGGGCAGCACCGTGGAAAAGACCGTGGC |
| | | CCCAACCGAGTGCAGC |

Antibody 024

| | | |
|---|---|---|
| 599 | HCDR1 | GGTFRDYAIS |
| | (Combined) | |
| 600 | HCDR2 | GIIPAFGTANYAQKFQG |
| | (Combined) | |
| 601 | HCDR3 | EQDPSYGYGGYPYEAMDV |
| | (Combined) | |
| 602 | HCDR1 | DYAIS |
| | (Kabat) | |
| 603 | HCDR2 | GIIPAFGTANYAQKFQG |
| | (Kabat) | |
| 604 | HCDR3 | EQDPSYGYGGYPYEAMDV |
| | (Kabat) | |
| 605 | HCDR1 | GGTFRDY |
| | (Chothia) | |
| 606 | HCDR2 | IPAFGT |
| | (Chothia) | |
| 607 | HCDR3 | EQDPSYGYGGYPYEAMDV |
| | (Chothia) | |
| 608 | VH | QVQLVQSGAEVKKPGSSVKVSCKASGGTFRDYAISWVRQAPGQ |
| | | GLEWMGGIIPAFGTANYAQKFQGRVTITADESTSTAYMELSSLRS |
| | | EDTAVYYCAREQDPSYGYGGYPYEAMDVWGQGTLVTVSS |
| 609 | VH DNA | CAGGTGCAATTGGTGCAGAGCGGTGCCGAAGTGAAAAAACCG |
| | | GGCAGCAGCGTGAAAGTTAGCTGCAAAGCATCCGGAGGGAC |
| | | GTTTCGTGACTACGCTATCTCTTGGGTGCGCCAGGCCCCGGG |
| | | CCAGGGCCTCGAGTGGATGGGCGGTATCATCCCGGCTTTCGG |
| | | CACTGCGAACTACGCCCAGAAATTTCAGGGCCGGGTGACCAT |
| | | TACCGCCGATGAAAGCACCAGCACCGCCTATATGGAACTGAG |
| | | CAGCCTGCGCAGCGAAGATACGGCCGTGTATTATTGCGCGCG |
| | | TGAACAGGACCCGAGCTACGGTTACGGTGGTTACCCGTATGA |
| | | AGCTATGGATGTTTGGGGCCAAGGCACCCTGGTGACTGTTAG |
| | | CTCA |
| 610 | Heavy Chain | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQDPSYGYGGYPYEAMDVWGQGTLVTVSSASTK |
| | | GPSVFPLAPSSKSTSGGTAALGCLVKDYFPEPVTVSWNSGALTS |
| | | GVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISR |
| | | TPEVTCVVVDVSHEDPEVKFNWYVDGVEVHNAKTKPREEQYAS |
| | | TYRVVSVLTVLHQDWLNGKEYKCKVSNKALPAPIEKTISKAKGQP |
| | | REPQVYTLPPSREEMTKNQVSLTCLVKGFYPSDIAVEWESNGQP |
| | | ENNYKTTPPVLDSDGSFFLYSKLTVQKSRWQQGNVFSCSVMHE |
| | | ALHNHYTQKSLSLSPGK |
| 611 | Heavy Chain | CAGGTGCAATTGGTGCAGAGCGGTGCCGAAGTGAAAAAACCG |
| | DNA | GGCAGCAGCGTGAAAGTTAGCTGCAAAGCATCCGGAGGGAC |
| | | GTTTCGTGACTACGCTATCTCTTGGGTGCGCCAGGCCCCGGG |
| | | CCAGGGCCTCGAGTGGATGGGCGGTATCATCCCGGCTTTCGG |
| | | CACTGCGAACTACGCCCAGAAATTTCAGGGCCGGGTGACCAT |
| | | TACCGCCGATGAAAGCACCAGCACCGCCTATATGGAACTGAG |
| | | CAGCCTGCGCAGCGAAGATACGGCCGTGTATTATTGCGCGCG |
| | | TGAACAGGACCCGAGCTACGGTTACGGTGGTTACCCGTATGA |
| | | AGCTATGGATGTTTGGGGCCAAGGCACCCTGGTGACTGTTAG |
| | | CTCAGCCTCCACCAAGGGTCCATCGGTCTTCCCCCTGGCACC |
| | | CTCCTCCAAGAGCACCTCTGGGGGCACAGCGGCCCTGGGCT |
| | | GCCTGGTCAAGGACTACTTCCCCGAACCGGTGACGGTGTCGT |
| | | GGAACTCAGGCGCCCTGACCAGCGGCGTGCACACCTTCCCG |
| | | GCTGTCCTACAGTCCTCAGGACTCTACTCCCTCAGCAGCGTG |
| | | GTGACCGTGCCCTCCAGCAGCTTGGGCACCCAGACCTACATC |
| | | TGCAACGTGAATCACAAGCCCAGCAACACCAAGGTGGACAAG |
| | | AGAGTTGAGCCCAAATCTTGTGACAAAACTCACACATGCCCAC |
| | | CGTGCCCAGCACCTGAACTCCTGGGGGGACCGTCAGTCTTCC |
| | | TCTTCCCCCCAAAACCCAAGGACACCCTCATGATCTCCCGGAC |
| | | CCCTGAGGTCACATGCGTGGTGGTGGACGTGAGCCACGAAGA |
| | | CCCTGAGGTCAAGTTCAACTGGTACGTGGACGGCGTGGAGGT |
| | | GCATAATGCCAAGACAAAGCCGCGGGAGGAGCAGTACGCCA |
| | | GCACGTACCGGGTGGTCAGCGTCCTCACCGTCCTGCACCAGG |
| | | ACTGGCTGAATGGCAAGGAGTACAAGTGCAAGGTCTCCAACA |
| | | AAGCCCTCCCAGCCCCCATCGAGAAAACCATCTCCAAAGCCA |
| | | AAGGGCAGCCCCGAGAACCACAGGTGTACACCCTGCCCCCAT |
| | | CCCGGGAGGAGATGACCAAGAACCAGGTCAGCCTGACCTGC |
| | | CTGGTCAAAGGCTTCTATCCCAGCGACATCGCCGTGGAGTGG |
| | | GAGAGCAATGGGCAGCCGGAGAACAACTACAAGACCACGCCT |
| | | CCCGTGCTGGACTCCGACGGCTCCTTCTTCCTCTACAGCAAG |
| | | CTCACCGTGGACAAGAGCAGGTGGCAGCAGGGGAACGTCTTC |
| | | TCATGCTCCGTGATGCATGAGGCTCTGCACAACCACTACACG |
| | | CAGAAGAGCCTCTCCCTGTCTCCGGGTAAA |
| 612 | LCDR1 | SGDNIPGHGVH |
| | (Combined) | |
| 613 | LCDR2 | DDTERPS |
| | (Combined) | |
| 614 | LCDR3 | SSWDSSMDSVV |
| | (Combined) | |
| 615 | LCDR1 | SGDNIPQHSVH |
| | (Kabat) | |
| 616 | LCDR2 | DDTERPS |
| | (Kabat) | |
| 617 | LCDR3 | SSWDSSMDSVV |
| | (Kabat) | |
| 618 | LCDR1 | DNIPQHS |
| | (Chothia) | |
| 619 | LCDR2 | DDT |
| | (Chothia) | |
| 620 | LCDR3 | WDSSMDSV |
| | (Chothia) | |
| 621 | VL | SYELTQPLSVSVALGQTARITCSGDNIPQHSVHVWQQKPGQAPV |
| | | LVIYDDTERPSGIPERFSGSNSGNTATLTISRAQAGDEADYYCSS |
| | | WDSSMDSWFGGGTKLTVL |
| 622 | VH DNA | AGCTACGAACTGACCCAGCCGCTGAGCGTGAGCGTGGCCCT |
| | | GGGCCAGACCGCGAGGATTACCTGTAGCGGCGATAACATCCC |
| | | GCAGCATTCTGTTCATTGGTACCAGCAGAAACCGGGCCAGGC |
| | | GCCGGTGCTGGTGATCTACGACGACACTGAACGTCCGAGCGG |
| | | CATCCCGGAACGTTTTAGCGGATCCAACAGCGGCAACACCGC |
| | | GACCCTGACCATTAGCAGGGCCCAGGCGGGCGACGAAGCGG |
| | | ATTATTACTGCTCTTCTTGGGACTCTTCTATGGACTCTGTTGTG |
| | | TTTGGCGGCGGCACGAAGTTAACCGTCCTA |
| 623 | Light Chain | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSVVFGGGTKLTVLGQPKAAPSVTLFPPSSEELQANKAT |
| | | LVCLISDFYPGAVTVAWKADSSPVKAGVETTTPSKQSNNKYAAS |
| | | SYLSLTPEGWKSHRSYSCQVTHEGSTVEKTVAPTECS |
| 624 | Light Chain | AGCTACGAACTGACCCAGCCGCTGAGCGTGAGCGTGGCCCT |
| | DNA | GGGCCAGACCGCGAGGATTACCTGTAGCGGCGATAACATCCC |
| | | GCAGCATTCTGTTCATTGGTACCAGCAGAAACCGGGCCAGGC |
| | | GCCGGTGCTGGTGATCTACGACGACACTGAACGTCCGAGCGG |
| | | CATCCCGGAACGTTTTAGCGGATCCAACAGCGGCAACACCGC |
| | | GACCCTGACCATTAGCAGGGCCCAGGCGGGCGACGAAGCGG |
| | | ATTATTACTGCTCTTCTTGGGACTCTTCTATGGACTCTGTTGTG |
| | | TTTGGCGGCGGCACGAAGTTAACCGTCCTAGGTCAGCCCAAG |
| | | GCTGCCCCCTCGGTCACTCTGTTCCCGCCCTCCTCTGAGGAG |
| | | CTTCAAGCCAACAAGGCCACACTGGTGTGTCTCATAAGTGACT |
| | | TCTACCCGGGAGCCGTGACAGTGGCCTGGAAGGCAGATAGCA |
| | | GCCCCGTCAAGGCGGGAGTGGAGACCACCACACCCTCCAAA |
| | | CAAAGCAACAACAAGTACGCGGCCAGCAGCTATCTGAGCCTG |
| | | ACGCCTGAGCAGTGGAAGTCCCACAGAAGCTACAGCTGCCAG |
| | | GTCACGCATGAAGGGAGCACCGTGGAGAAGACAGTGGCCCC |
| | | TACAGAATGTTCA |

Antibody 025

| | | |
|---|---|---|
| 625 | HCDR1 | GGTFRDYAIS |
| | (Combined) | |
| 626 | HCDR2 | GIIPAFGTANYAQKFQG |
| | (Combined) | |
| 627 | HCDR3 | EQSPEYGYGGYPYEAMDV |
| | (Combined) | |
| 628 | HCDR1 | DYAIS |
| | (Kabat) | |
| 629 | HCDR2 | GIIPAFGTANYAQKFQG |
| | (Kabat) | |
| 630 | HCDR3 | EQSPEYGYGGYPYEAMDV |
| | (Kabat) | |
| 631 | HCDR1 | GGTFRDY |
| | (Chothia) | |
| 632 | HCDR2 | IPAFGT |
| | (Chothia) | |
| 633 | HCDR3 | EQSPEYGYGGYPYEAMDV |
| | (Chothia) | |
| 634 | VH | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREGSPEYGYGGYPYEAMDVWGQGTLVTVSS |
| 635 | VH DNA | CAGGTGCAGCTGGTGCAGTCAGGCGCCGAAGTGAAGAAACC |
| | | CGGCTCTAGCGTGAAAGTCAGCTGTAAAGCTAGTGGCGGCAC |
| | | CTTTAGAGACTACGCTATTAGCTGGGTCAGACAGGCCCCAGG |
| | | TCAGGGCCTGGAGTGGATGGGCGGAATTATCCCCGCCTTCGG |
| | | CACCGCTAACTACGCTCAGAAATTTCAGGGTAGAGTGACTATC |
| | | ACCGCCGACGAGTCTACTAGCACCGCCTATATGGAACTGTCTA |
| | | GCCTGAGATCAGAGGACACCGCCGTCTACTACTGCGCTAGAG |
| | | AGCAGTCCCCCGAGTACGGCTACGGCGGCTACCCCTACGAG |
| | | GCTATGGACGTGTGGGGTCAGGGCACCCTGGTCACCGTGTCT |
| | | AGC |
| 636 | Heavy Chain | QVQLVQSGAEVKKPGSSVKVSCKASGGTFRDYAISVWRQAPGQ |
| | | GLEWMGGIIPAFGTANYAQKFQGRVTITADESTSTAYMELSSLRS |
| | | EDTAVYYCAREQSPEYGYGGYPYEAMDVWGQGTLVTVSSASTK |
| | | GPSVFPLAPSSKSTSGGTAALGCLVKDYFPEPVTVSWNSGALTS |
| | | GVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISR |
| | | TPEVTCVVVDVSHEDPEVKFNWYVDGVEVHNAKTKPREEQYNS |
| | | TYRVVSVLTVLHQDWLNGKEYKCKVSNKALPAPIEKTISKAKGQP |
| | | REPQVYTLPPSREEMTKNQVSLTCLVKGFYPSDIAVEWESNGQP |
| | | ENNYKTTPPVLDSDGSFFLYSKLTVDKSRWQQGNVFSCSVMHE |
| | | ALHNHYTQKSLSLSPGK |
| 637 | Heavy Chain | CAGGTGCAGCTGGTGCAGTCAGGCGCCGAAGTGAAGAAACC |
| | DNA | CGGCTCTAGCGTGAAAGTCAGCTGTAAAGCTAGTGGCGGCAC |
| | | CTTTAGAGACTACGCTATTAGCTGGGTCAGACAGGCCCCAGG |
| | | TCAGGGCCTGGAGTGGATGGGCGGAATTATCCCCGCCTTCGG |
| | | CACCGCTAACTACGCTCAGAAATTTCAGGGTAGAGTGACTATC |
| | | ACCGCCGACGAGTCTACTAGCACCGCCTATATGGAACTGTCTA |
| | | GCCTGAGATCAGAGGACACCGCCGTCTACTACTGCGCTAGAG |
| | | AGCAGTCCCCCGAGTACGGCTACGGCGGCTACCCCTACGAG |
| | | GCTATGGACGTGTGGGGTCAGGGCACCCTGGTCACCGTGTCT |
| | | AGCGCTAGCACTAAGGGCCCAAGTGTGTTTCCCCTGGCCCCC |
| | | AGCAGCAAGTCTACTTCCGGCGGAACTGCTGCCCTGGGTTGC |
| | | CTGGTGAAGGACTACTTCCCCGAGCCCGTGACAGTGTCCTGG |
| | | AACTCTGGGGCTCTGACTTCCGGCGTGCACACCTTCCCCGCC |
| | | GTGCTGCAGAGCAGCGGCCTGTACAGCCTGAGCAGCGTGGT |
| | | GACAGTGCCCTCCAGCTCTCTGGGAACCCAGACCTATATCTG |
| | | CAACGTGAACCACAAGCCCAGCAACACCAAGGTGGACAAGAG |
| | | AGTGGAGCCCAAGAGCTGCGACAAGACCCACACCTGCCCCCC |
| | | CTGCCCAGCTCCAGAACTGCTGGGAGGGCCTTCCGTGTTCCT |
| | | GTTCCCCCCCAAGCCCAAGGACACCCTGATGATCAGCAGGAC |
| | | CCCCGAGGTGACCTGCGTGGTGGTGGACGTGTCCCACGAGG |
| | | ACCCAGAGGTGAAGTTCAACTGGTACGTGGACGGCGTGGAGG |
| | | TGCACAACGCCAAGACCAAGCCCAGAGAGGAGCAGTACAACA |
| | | GCACCTACAGGGTGGTGTCCGTGCTGACCGTGCTGCACCAGG |
| | | ACTGGCTGAACGGCAAAGAATACAAGTGCAAAGTCTCCAACAA |
| | | GGCCCTGCCAGCCCCAATCGAAAAGACAATCAGCAAGGCCAA |
| | | GGGCCAGCCACGGGAGCCCCAGGTGTACACCCTGCCCCCCA |
| | | GCCGGGAGGAGATGACCAAGAACCAGGTGTCCCTGACCTGTC |
| | | TGGTGAAGGGCTTCTACCCCAGCGATATCGCCGTGGAGTGGG |
| | | AGAGCAACGGCCAGCCCGAGAACAACTACAAGACCACCCCCC |
| | | CAGTGCTGGACAGCGACGGCAGCTTCTTCCTGTACAGCAAGC |
| | | TGACCGTGGACAAGTCCAGGTGGCAGCAGGGCAACGTGTTCA |
| | | GCTGCAGCGTGATGCACGAGGCCCTGCACAACCACTACACCC |
| | | AGAAGTCCCTGAGCCTGAGCCCCGGCAAG |
| 638 | LCDR1 | SGDNIPQHSVH |
| | (Combined) | |
| 639 | LCDR2 | DDTERPS |
| | (Combined) | |
| 640 | LCDR3 | SSWDSSMDSVV |
| | (Combined) | |
| 641 | LCDR1 | SGDNIPQHSVH |
| | (Kabat) | |
| 642 | LCDR2 | DDTERPS |
| | (Kabat) | |
| 643 | LCDR3 | SSWDSSMDSVV |
| | (Kabat) | |
| 644 | LCDR1 | DNIPQHS |
| | (Chothia) | |
| 645 | LCDR2 | DDT |
| | (Chothia) | |
| 646 | LCDR3 | WDSSMDSV |
| | (Chothia) | |
| 647 | VL | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSVVFGGGTKLTVL |
| 648 | VH DNA | AGCTACGAGCTGACTCAGCCCCTGAGCGTCAGCGTGGCCCTG |
| | | GGTCAGACCGCTAGAATCACCTGTAGCGGCGATAATATCCCT |
| | | CAGCACTCAGTGCACTGGTATCAGCAGAAGCCCGGTCAGGCC |
| | | CCCGTGCTGGTGATCTACGACGACACCGAGCGGCCTAGCGG |
| | | AATCCCCGAGCGGTTTAGCGGCTCTAATAGCGGTAACACCGC |
| | | TACCCTGACTATCTCTAGGGCTCAGGCCGGCGACGAGGCCGA |
| | | CTACTACTGCTCTAGCTGGGATAGCTCTATGGATAGCGTGGTG |
| | | TTCGGCGGAGGCACTAAGCTGACCGTGCTG |
| 649 | Light Chain | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSVVFGGGTKLTVLGQPKAAPSVTLFPPSSEELQANKAT |
| | | LVCLISDFYPGAVTVAWKADSSPVKAGVETTTPSKQSNNKYAAS |
| | | SYLSLTPEQWKSHRSYSCQVTHEGSTVEKTVAPTECS |
| 650 | Light Chain | AGCTACGAGCTGACTCAGCCCCTGAGCGTCAGCGTGGCCCTG |
| | DNA | GGTCAGACCGCTAGAATCACCTGTAGCGGCGATAATATCCCT |
| | | CAGCACTCAGTGCACTGGTATCAGCAGAAGCCCGGTCAGGCC |
| | | CCCGTGCTGGTGATCTACGACGACACCGAGCGGCCTAGCGG |
| | | AATCCCCGAGCGGTTTAGCGGCTCTAATAGCGGTAACACCGC |
| | | TACCCTGACTATCTCTAGGGCTCAGGCCGGCGACGAGGCCGA |
| | | CTACTACTGCTCTAGCTGGGATAGCTCTATGGATAGCGTGGTG |
| | | TTCGGCGGAGGCACTAAGCTGACCGTGCTGGGTCAGCCTAAG |
| | | GCTGCCCCCAGCGTGACCCTGTTCCCCCCCAGCAGCGAGGA |
| | | GCTGCAGGCCAACAAGGCCACCCTGGTGTGCCTGATCAGCGA |
| | | CTTCTACCCAGGCGCCGTGACCGTGGCCTGGAAGGCCGACA |
| | | GCAGCCCCGTGAAGGCCGGCGTGGAGACCACCACCCCCAGC |
| | | AAGCAGAGCAACAACAAGTACGCCGCCAGCAGCTACCTGAGC |
| | | CTGACCCCCGAGCAGTGGAAGAGCCACAGGTCCTACAGCTGC |
| | | CAGGTGACCCACGAGGGCAGCACCGTGGAAAAGACCGTGGC |
| | | CCCAACCGAGTGCAGC |

Antibody 026

| | | |
|---|---|---|
| 651 | HCDR1 | GGTFRDYAIS |
| | (Combined) | |
| 652 | HCDR2 | GIIPAFGTANYAQKFQG |
| | (Combined) | |
| 653 | HCDR3 | EQSPEYGYGGYPYEAMDV |
| | (Combined) | |
| 654 | HCDR1 | DYAIS |
| | (Kabat) | |
| 655 | HCDR2 | GIIPAFGTANYAQKFQG |
| | (Kabat) | |
| 656 | HCDR3 | EQSPEYGYGGYPYEAMDV |
| | (Kabat) | |
| 657 | HCDR1 | GGTFRDY |
| | (Chothia) | |
| 658 | HCDR2 | IPAFGT |
| | (Chothia) | |
| 659 | HCDR3 | EQSPEYGYGGYPYEAMDV |
| | (Chothia) | |
| 660 | VH | QVQLVQSGAEVKKPGSSVKVSCKASGGTFRDYAISWVRQAPGQ |
| | | GLEWMGGIIPAFGTANYAQKFQGRVTITADESTSTAYMELSSLRS |
| | | EDTAVYYCAREQSPEYGYGGYPYEAMDVWGQGTLVTVSS |
| 661 | VH DNA | CAGGTGCAATTGGTGCAGAGCGGTGCCGAAGTGAAAAAACCG |
| | | GGCAGCAGCGTGAAAGTTAGCTGCAAAGCATCCGGAGGGAC |
| | | GTTTCGTGACTACGCTATCTCTTGGGTGCGCCAGGCCCCGGG |
| | | CCAGGGCCTCGAGTGGATGGGCGGTATCATCCCGGCTTTCGG |
| | | CACTGCGAACTACGCCCAGAAATTTCAGGGCCGGGTGACCAT |
| | | TACCGCCGATGAAAGCACCAGCACCGCCTATATGGAACTGAG |
| | | CAGCCTGCGCAGCGAAGATACGGCCGTGTATTATTGCGCGCG |
| | | TGAACAGAGCCCGGAATACGGTTACGGTGGTTACCCGTATGA |
| | | AGCTATGGATGTTTGGGGCCAAGGCACCCTGGTGACTGTTAG |
| | | CTCA |
| 662 | Heavy Chain | QVQLVQSGAEVKKPGSSVKVSCKASGGTFRDYAISWVRQAPGQ |
| | | GLEWMGGIIPAFGTANYAQKFQGRVTITADESTSTAYMELSSLRS |
| | | EDTAVYYCAREQSPEYGYGGYPYEAMDVWGQGTLVTVSSASTK |
| | | GPSVFPLAPSSKSTSGGTAALGCLVKDYFPEPVTVSWNSGALTS |
| | | GVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISR |
| | | TPEVTCVVVDVSHEDPEVKFNWYVDGVEVHNAKTKPREEQYAS |
| | | TYRVVSVLTVLHQDWLNGKEYKCKVSNKALPAPIEKTISKAKGQP |
| | | REPQVYTLPPSREEMTKNQVSLTCLVKGFYPSDIAVEWESNGQP |
| | | ENNYKTTPPVLDSDGSFFLYSKLTVQKSRWQQGNVFSCSVMHE |
| | | ALHNHYTQKSLSLSPGK |
| 663 | Heavy Chain | CAGGTGCAATTGGTGCAGAGCGGTGCCGAAGTGAAAAAACCG |
| | DNA | GGCAGCAGCGTGAAAGTTAGCTGCAAAGCATCCGGAGGGAC |
| | | GTTTCGTGACTACGCTATCTCTTGGGTGCGCCAGGCCCCGGG |
| | | CCAGGGCCTCGAGTGGATGGGCGGTATCATCCCGGCTTTCGG |
| | | CACTGCGAACTACGCCCAGAAATTTCAGGGCCGGGTGACCAT |
| | | TACCGCCGATGAAAGCACCAGCACCGCCTATATGGAACTGAG |
| | | CAGCCTGCGCAGCGAAGATACGGCCGTGTATTATTGCGCGCG |
| | | TGAACAGAGCCCGGAATACGGTTACGGTGGTTACCCGTATGA |
| | | AGCTATGGATGTTTGGGGCCAAGGCACCCTGGTGACTGTTAG |
| | | CTCAGCCTCCACCAAGGGTCCATCGGTCTTCCCCCTGGCACC |
| | | CTCCTCCAAGAGCACCTCTGGGGGCACAGCGGCCCTGGGCT |
| | | GCCTGGTCAAGGACTACTTCCCCGAACCGGTGACGGTGTCGT |
| | | GGAACTCAGGCGCCCTGACCAGCGGCGTGCACACCTTCCCG |
| | | GCTGTCCTACAGTCCTCAGGACTCTACTCCCTCAGCAGCGTG |
| | | GTGACCGTGCCCTCCAGCAGCTTGGGCACCCAGACCTACATC |
| | | TGCAACGTGAATCACAAGCCCAGCAACACCAAGGTGGACAAG |
| | | AGAGTTGAGCCCAAATCTTGTGACAAAACTCACACATGCCCAC |
| | | CGTGCCCAGCACCTGAACTCCTGGGGGGACCGTCAGTCTTCC |
| | | TCTTCCCCCCAAAACCCAAGGACACCCTCATGATCTCCCGGAC |
| | | CCCTGAGGTCACATGCGTGGTGGTGGACGTGAGCCACGAAGA |
| | | CCCTGAGGTCAAGTTCAACTGGTACGTGGACGGCGTGGAGGT |
| | | GCATAATGCCAAGACAAAGCCGCGGGAGGAGCAGTACGCCA |
| | | GCACGTACCGGGTGGTCAGCGTCCTCACCGTCCTGCACCAGG |
| | | ACTGGCTGAATGGCAAGGAGTACAAGTGCAAGGTCTCCAACA |
| | | AAGCCCTCCCAGCCCCCATCGAGAAAACCATCTCCAAAGCCA |
| | | AAGGGCAGCCCCGAGAACCACAGGTGTACACCCTGCCCCCAT |
| | | CCCGGGAGGAGATGACCAAGAACCAGGTCAGCCTGACCTGC |
| | | CTGGTCAAAGGCTTCTATCCCAGCGACATCGCCGTGGAGTGG |
| | | GAGAGCAATGGGCAGCCGGAGAACAACTACAAGACCACGCCT |
| | | CCCGTGCTGGACTCCGACGGCTCCTTCTTCCTCTACAGCAAG |
| | | CTCACCGTGGACAAGAGCAGGTGGCAGCAGGGGAACGTCTTC |
| | | TCATGCTCCGTGATGCATGAGGCTCTGCACAACCACTACACG |
| | | CAGAAGAGCCTCTCCCTGTCTCCGGGTAAA |
| 664 | LCDR1 | SGDNIPQHSVH |
| | (Combined) | |
| 665 | LCDR2 | DDTERPS |
| | (Combined) | |
| 666 | LCDR3 | SSWDSSMDSVV |
| | (Combined) | |
| 667 | LCDR1 | SGDNIPQHSVH |
| | (Kabat) | |
| 668 | LCDR2 | DDTERPS |
| | (Kabat) | |
| 669 | LCDR3 | SSWDSSMDSVV |
| | (Kabat) | |
| 670 | LCDR1 | DNIPQHS |
| | (Chothia) | |
| 671 | LCDR2 | DDT |
| | (Chothia) | |
| 672 | LCDR3 | WDSSMDSV |
| | (Chothia) | |
| 673 | VL | SYELTQPLSVSVALGQTARITCSGDNIPQHSVHWYQQKPGQAPV |
| | | LVIYDDTERPSGIPERFSGSNSGNTATLTISRAQAGDEADYYCSS |
| | | WDSSMDSVVFGQQTKLTVL |
| 674 | VH DNA | AGCTACGAACTGACCCAGCCGCTGAGCGTGAGCGTGGCCCT |
| | | GGGCCAGACCGCGAGGATTACCTGTAGCGGCGATAACATCCC |
| | | GCAGCATTCTGTTCATTGGTACCAGCAGAAACCGGGCCAGGC |
| | | GCCGGTGCTGGTGATCTACGACGACACTGAACGTCCGAGCGG |
| | | CATCCCGGAACGTTTTAGCGGATCCAACAGCGGCAACACCGC |
| | | GACCCTGACCATTAGCAGGGCCCAGGCGGGCGACGAAGCGG |
| | | ATTATTACTGCTCTTCTTGGGACTCTTCTATGGACTCTGTTGTG |
| | | TTTGGCGGCGGCACGAAGTTAACCGTCCTA |
| 675 | Light Chain | SYELTQPLSVSVALGQTARITCSGDNIPQHSVHWYQQKPGQAPV |
| | | LVIYDDTERPSGIPERFSGSNSGNTATLTISRAQAGQEADYYCSS |
| | | WDSSMDSWFGGGTKLTVLGQPKAAPSVTLFPPSSEELQANKAT |
| | | LVCLISDFYPGAVTVAWKADSSPVKAGVETTTPSKQSNNKYAAS |
| | | SYLSLTPEQWKSHRSYSCQVTHEGSTVEKTVAPTECS |
| 676 | Light Chain | AGCTACGAACTGACCCAGCCGCTGAGCGTGAGCGTGGCCCT |
| | DNA | GGGCCAGACCGCGAGGATTACCTGTAGCGGCGATAACATCCC |
| | | GCAGCATTCTGTTCATTGGTACCAGCAGAAACCGGGCCAGGC |
| | | GCCGGTGCTGGTGATCTACGACGACACTGAACGTCCGAGCGG |
| | | CATCCCGGAACGTTTTAGCGGATCCAACAGCGGCAACACCGC |
| | | GACCCTGACCATTAGCAGGGCCCAGGCGGGCGACGAAGCGG |
| | | ATTATTACTGCTCTTCTTGGGACTCTTCTATGGACTCTGTTGTG |
| | | TTTGGCGGCGGCACGAAGTTAACCGTCCTAGGTCAGCCCAAG |
| | | GCTGCCCCCTCGGTCACTCTGTTCCCGCCCTCCTCTGAGGAG |
| | | CTTCAAGCCAACAAGGCCACACTGGTGTGTCTCATAAGTGACT |
| | | TCTACCCGGGAGCCGTGACAGTGGCCTGGAAGGCAGATAGCA |
| | | GCCCCGTCAAGGCGGGAGTGGAGACCACCACACCCTCCAAA |
| | | CAAAGCAACAACAAGTACGCGGCCAGCAGCTATCTGAGCCTG |
| | | ACGCCTGAGCAGTGGAAGTCCCACAGAAGCTACAGCTGCCAG |
| | | GTCACGCATGAAGGGAGCACCGTGGAGAAGACAGTGGCCCC |
| | | TACAGAATGTTCA |

Antibody 027

| | | |
|---|---|---|
| 677 | HCDR1 | SYGIH |
| 678 | HCDR2 | VIGYDGSDKNYADSVKG |
| 679 | HCDR3 | DQLGDAFDI |
| 680 | VH | QVQLVESGGG VVQPGRSLRL SCAASGFTFS SYGIHWVRQA |
| | | PGKGLEWVAV IGYDGSDKNY ADSVKGRFTI FRDNSKNTLY |
| | | LQMNSLRAED TAVYYCARDQ LGDAFDIWGQ GTMVTVSS |
| 681 | LCDR1 | KASQSVSSSLA |
| 682 | LCDR2 | DASNRAT |
| 683 | LCDR3 | QQRSNWPPYT |
| 684 | VL | EIVLTQSPAT LSLSPGERAT LSCKASQSVS SSLAWYQQKP |
| | | GQAPRLLIYD ASNRATGIPA RFSGSGSGTD FTLTISSLEP |
| | | EDFAVYYCQQ RSNWPPYTFG QGTKLEIKR |

Antibody 028

| | | |
|---|---|---|
| 685 | HCDR1 | SYGIS |
| 686 | HCDR2 | WISAYNGNTKYAQKLQG |
| 687 | HCDR3 | DSAAHGMDV |
| 688 | VH | QVQLVQSGGE VKKPGASVKV SCKTSGYTFT SYGISWVRQA |
| | | PGQGLEWMGW ISAYNGNTKY QKLQGRLTM TTDTSTTTAY |
| | | MELRSLRSDD TAVYYCARDS AAHGMDVWGQ GTTVTVSS |
| 689 | LCDR1 | RASQGISSWLA |
| 690 | LCDR2 | AASSLQS |
| 691 | LCDR3 | QQYNSYPYT |
| 692 | VL | DIQMTQSPSS LSASVGDRVT ITCRASQGIS SWLAWYQQKP |
| | | EKAPKSLIYA ASSLQSGVPS RFRGSGSGTD FTLTISSLQP |
| | | EDFATYYCQQ YNSYPYTFGQ GTKLEIKR |

Antibody 029

| | | |
|---|---|---|
| 693 | HCDR1 | SYGLS |
| 694 | HCDR2 | WISPYNGNTHYAQKLQG |
| 695 | HCDR3 | ASAAHGMDV |
| 696 | VH | QVQLVQSGAE VKKPGASVKV SCKASGYTFT SYGLSWVRQA |
| | | PGQGLEWMGW ISPYNGNTHY AQKLQGRVTM TTDTSTSTAD |
| | | MDLRSLRSDD TAVYYCARAS AAHGMDVWGQ GTTVTVSS |
| 697 | LCDR1 | RASQGISSWLA |
| 698 | LCDR2 | AASSLQS |
| 699 | LCDR3 | QQYNSYPYT |
| 700 | VL | DIQMTQSPSS LSASVGDRVT ITCRASQGIS SWLAWYQQKP |
| | | EKAPKSLIYA ASSLQSGVPS RFSGSRSGTD FTLTISSLQP |
| | | EDFATYYCQQ YNSYPYTFGQ GTKLEIKR |

Antibody 030

| | | |
|---|---|---|
| 701 | HCDR1 | SYGLS |
| 702 | HCDR2 | WISPYNGNTHYAQKLQG |
| 703 | HCDR3 | DSAAHGMDV |
| 704 | VH | QVQLVQSGAE VKKPGASVKV SCKASGYTFT SYGLSWVRQA |
| | | PGQGLEWMGW ISPYNGNTHY AQKLQGRVTM TTDTSTSTAY |
| | | MDLRSLRSDD TAVYYCARDS AAHGMDVWGQ GTTVTVSS |
| 705 | LCDR1 | RASQGISSWLA |
| 706 | LCDR2 | AASSLQS |
| 707 | LCDR3 | QQYNSYPYT |
| 708 | VL | DIQMTQSPSS LSASVGDRVT ITCRASQGIS SWLAWYQQKP |
| | | EKAPKSLIYA ASSLQSGVPS RFSGSRSGTD FTLTISSLQP |
| | | EDFATYYCQQ YNSYPYTFGQ GTKLEIKR |

Antibody 031

| | | |
|---|---|---|
| 709 | HCDR1 | SYGLS |
| 710 | HCDR2 | WISAYNGNTNYAQKLQG |
| 711 | HCDR3 | DSAAHGMDV |
| 712 | VH | QVQLVQSGAE VKKPGASVKV SCKASGYTFT SYGLSWVRQA |
| | | PGQGLEWMGW ISAYNGNTNY AQKLQGRVTM TTDTSTSTAY |
| | | MDLRSLRSDD TAVYYCARDS AAHGMDVWGQ GTTVTVSS |
| 713 | LCDR1 | RASQGISSWLA |
| 714 | LCDR2 | AASSLQS |
| 715 | LCDR3 | QQYNSYPYT |
| 716 | VL | DIQMTQSPSS LSASVGDRVT ITCRASQGIS SWLAWYQQKP |
| | | EKAPKSLIYA ASSLQSGVPS RFSGSRSGTD FTLTISSLQP |
| | | EDFATYYCQQ YNSYPYTFGQ GTKLEIKR |

Antibody 032

| | | |
|---|---|---|
| 717 | HCDR1 | SYGIS |
| 718 | HCDR2 | WISAYNGNTKYAQKLQG |
| 719 | HCDR3 | DSAAHGMDV |
| 720 | VH | QVQVVQSGAE VKKPGASVKV SCKTSGYTFT SYGISWVRQA |
| | | PGQGLEWMGW ISAYNGNTKY AQKLQGRLTM TTDTSTTTAY |
| | | MELRSLRSDD TAVYYCARDS AAHGMDVWGQ GTTVSVSS |

Antibody 033

| | | |
|---|---|---|
| 721 | HCDR1 | NFVMS |
| 722 | HCDR2 | GISGSGGNTDHADSVKG |
| 723 | HCDR3 | DSGGLFDY |
| 724 | VH | EVQLLESGGG LVQPGGSLRL SCAASGFTFS NFVMSWVRQA |
| | | PGKGLEWVSG ISGSGGNTDH ADSVKGRFTI SRDNSKNTVY |
| | | LQMNSLRAED TAVYYCAKDS GGLFDYWGLG TLVTVSS |
| 725 | LCDR1 | RASQSVSSYLA |
| 726 | LCDR2 | DASNRAT |
| 727 | LCDR3 | QQRSNWPHLT |
| 728 | VL | EIVLTQSPAT LSLSPGERAT LSCRASQSVS SYLAWYQQKP |
| | | GQAPRLLIYD ASNRATGIPA RFSGSGSRTD FTLTISSLEP |
| | | EDFAVYYCQQ RSNWPHLTFG GGTKVEIKR |

Antibody 034

| | | |
|---|---|---|
| 729 | HCDR1 | TYGMH |
| 730 | HCDR2 | VISHDGSDKYYADSVKG |
| 731 | HCDR3 | DQSIIETFDY |
| 732 | VH | QVQLVESGGG VVQPGRSLRL SCAASGFTFS TYGMHWVRQA |
| | | PGKGLEWVAV ISHDGSDKYY ADSVKGRFTI SRDNSKNTLY |
| | | LQMNSLRAED TAVYYCARDQ SIIETFDYWG QGTLVTVSS |
| 733 | LCDR1 | RASQSVSSYLA |
| 734 | LCDR2 | DASNRAT |
| 735 | LCDR3 | QQRSNWGFT |
| 736 | VL | EIVLTQSPAT LSLSPGERAT LSCRASQSVS SYLAWYQQKP |
| | | GQAPRLLIYD ASNRATGIPA RFSGSGSGTD FTLTISSLEP |
| | | EDFAVYYCQQ RSNWGFTFGP GTKVDIKR |

Antibody 035

| | | |
|---|---|---|
| 737 | HCDR1 | SYGMH |
| 738 | HCDR2 | VIWYDGSIKYYADSVKG |
| 739 | HCDR3 | EGGRDAFDI |
| 740 | VH | QVQLVESGGG VVQPGRSLRL SCAVSGFTFR SYGMHWVRQA |
| | | PGKGLEWVAV IWYDGSIKYY ADSVKGRFTI SRDNSKNTLY |
| | | LQMNSLRAED TAVYFCAREG GRDAFDIWGQ GTMVTVSS |
| 741 | LCDR1 | RASQGISSALA |
| 742 | LCDR2 | DASSLES |
| 743 | LCDR3 | QQFNSYPHT |
| 744 | VL | AVQLTQSPSS LSASVGDRVT ITCRASQGIS SALAWYQQKP |
| | | GKAPKLLIYD ASSLESGVPS RFSGSGSGTD FTLTISSLQP |
| | | EDFATYCCQQ FNSYPHTFGG GTKVEIKR |

Antibody 36

| | | |
|---|---|---|
| 745 | HCDR1 | SYAMS |
| 746 | HCDR2 | AISDSGGSTYYADSVKG |
| 747 | HCDR3 | EIAVALFDY |
| 748 | VH | EVQLLESGGG LVQPGGSLRL SCAASGFTFS SYAMSWVRQA |
| | | PGKGLEWVSA ISDSGGSTYY ADSVKGRFTI SRDNSKNTLY |
| | | LQMNSLRAED TAAYYCAKEI AVALFDYWGQ GTLVTVSS |
| 749 | LCDR1 | RASQSVSSYLA |
| 750 | LCDR2 | DASNRAT |
| 751 | LCDR3 | QQRSSWPPYT |
| 752 | VL | EIVLTQSPAT LSLSPGERAT LSCRASQSVS SYLAWYQQKP |
| | | GQAPRLLIYD ASNRATGIPA RFSGSGSGTD FTLTISSLEP |
| | | EDFAVYYCQQ RSSWPPYTFG QGTKLEIKR |

Antibody 037

| | | |
|---|---|---|
| 753 | VH | QVQLVQSGAEVKKPGSSVKVSCKASGGTFRDYAISWVRQAPGQ |
| | | GLEWMGGIIPAFGTANYAQKFQGRVTITADESTSTAYMELSSLRS |
| | | EDTAVYYCAREQDPEYGYGGYPYEAMDVWGQGTLVTVSS |
| 754 | VL | SYELTQPLSVSVALGQTARITCSGDNIPQHSVHWYQQKPGQAPVL |
| | | VIYDDTERPSGIPERFSGSNSGNTATLTISRAQAGDEADYYCSSWD |
| | | SSMDSVVFGGGTKLTVL |

Antibody 038

| | | |
|---|---|---|
| 755 | VH | QVQLVQSGAEVKKPGSSVKVSCKASGGTFRDYAISWVRQAPGQ |
| | | GLEWMGGIIPAFGTANYAQKFQGRVTITADESTSTAYMELSSLRS |
| | | EDTAVYYCAREQDPESGYGGYPYEAMDVWGQGTLVTVSS |
| 756 | VL | SYELTQPLSVSVALGQTARITCSGDNIPQHSVHWYQQKPGQAPVL |
| | | VIYDDTERPSGIPERFSGSNSGNTATLTISRAQAGDEADYYCSSWD |
| | | SSMDSVVFGGGTKLTVL |

Antibody 039

| | | |
|---|---|---|
| 762 | HCDR1 | DAWMN |
| 763 | HCDR2 | EIRNKAKNHATYYAESVIG |
| 764 | HCDR3 | GALGLDY |
| 765 | LCDR1 | RASQEISGYLS |
| 766 | LCDR2 | AASTLNS |
| 767 | LCDR3 | LQYFSYPLT |

Antibody 040

| | | |
|---|---|---|
| 768 | HCDR1 | DYYMN |
| 769 | HCDR2 | LIGWDGGSTYYADSVKG |
| 770 | HCDR3 | AYSGYELDY |
| 771 | LCDR1 | SGSSSNIGNNAVN |
| 772 | LCDR2 | DNNNRPS |
| 773 | LCDR3 | AAWDDSLNASI |

Antibody 041

| | | |
|---|---|---|
| 774 | HCDR1 | SYGMH |
| 775 | HCDR2 | FTRYDGSNKYYADSVRG |
| 776 | HCDR3 | ENIDAFDV |
| 777 | LCDR1 | SGSSSNIGNNAVN |
| 778 | LCDR2 | DNQQRPS |
| 779 | LCDR3 | WDDRLFGPV |

Antibody 042

| | | |
|---|---|---|
| 780 | HCDR1 | SYAMS |
| 781 | HCDR2 | SISDSGAGRYYADSVEG |
| 782 | HCDR3 | THDSGELLDAFDI |
| 783 | LCDR1 | SGSSSNIGSNHVL |
| 784 | LCDR2 | GNSNRPS |
| 785 | LCDR3 | AAWDDSLNGWV |

Antibody 043

| | | |
|---|---|---|
| 786 | HCDR1 | TYAMN |
| 787 | HCDR2 | VISYDGSNKNYVDSVKG |
| 788 | HCDR3 | NFDNSGYAIPDAFDI |
| 789 | LCDR1 | TGSSSNIGAGYDVH |
| 790 | LCDR2 | DNNSRPS |
| 791 | LCDR3 | AAWDDSLGGPV |

Antibody 044

| | | |
|---|---|---|
| 792 | HCDR1 | NAWMS |
| 793 | HCDR2 | YISRDADITHYPASVKG |
| 794 | HCDR3 | GFDYAGDDAFDI |
| 795 | LCDR1 | SGSSSNIGSNAVN |
| 796 | LCDR2 | GNSDRPS |
| 797 | LCDR3 | AAWDDSLNGRWV |

Antibody 045

| | | |
|---|---|---|
| 798 | HCDR1 | DYYMS |
| 799 | HCDR2 | LIGHDGNNKYYLDSLEG |
| 800 | HCDR3 | ATDSGYDLLY |
| 801 | LCDR1 | SGSSSNIGNNAVN |
| 802 | LCDR2 | YDDLLPS |
| 803 | LCDR3 | TTWDDSLSGVV |

Antibody 046

| | | |
|---|---|---|
| 804 | HCDR1 | DYYMS |
| 805 | HCDR2 | AIGFSDDNTYYADSVKG |
| 806 | HCDR3 | GDGSGWSF |
| 807 | LCDR1 | SGSSSNIGNNAVN |
| 808 | LCDR2 | DNNKRPS |
| 809 | LCDR3 | ATWDDSLRGWV |

Antibody 047

| | | |
|---|---|---|
| 810 | HCDR1 | NYGMH |
| 811 | HCDR2 | VISYDGSNKYYADSVKG |
| 812 | HCDR3 | WRDAFDI |
| 813 | LCDR1 | TGSSSNIGAGYDVH |
| 814 | LCDR2 | SDNQRPS |
| 815 | LCDR3 | AAWDDSLSGSWV |

Antibody 048

| | | |
|---|---|---|
| 816 | HCDR1 | TYGMH |
| 817 | HCDR2 | VISYDGSNKYYADSVKG |
| 818 | HCDR3 | ENFDAFDV |
| 819 | LCDR1 | TGSSSNIGAGYDVH |
| 820 | LCDR2 | SNSQRPS |
| 821 | LCDR3 | AAWDDSLNGQVV |

Antibody 049

| | | |
|---|---|---|
| 822 | HCDR1 | TYGMH |
| 823 | HCDR2 | VIAYDGSKKDYADSVKG |
| 824 | HCDR3 | EYRDAFDI |
| 825 | LCDR1 | TGSSSNIGAGYDVH |
| 826 | LCDR2 | GNSNRPS |
| 827 | LCDR3 | AAWDDSVSGWM |

Antibody 050

| | | |
|---|---|---|
| 828 | HCDR1 | SYGMH |
| 829 | HCDR2 | VISYDGINKDYADSMKG |
| 830 | HCDR3 | ERKDAFDI |
| 831 | LCDR1 | TGSSSNIGAGYDVH |
| 832 | LCDR2 | SNNQRPS |
| 833 | LCDR3 | ATWDDSLNGLV |

Antibody 051

| | | |
|---|---|---|
| 834 | HCDR1 | NYGMH |
| 835 | HCDR2 | VISYDGSNRYYADSVKG |
| 836 | HCDR3 | DRWNGMDV |
| 837 | LCDR1 | SGSSSNIGAGYDVH |
| 838 | LCDR2 | ANNQRPS |
| 839 | LCDR3 | AAWDDSLNGPWV |

Antibody 052

| | | |
|---|---|---|
| 840 | HCDR1 | SYGMH |
| 841 | HCDR2 | VISYDGSDTAYADSVKG |
| 842 | HCDR3 | DHSVIGAFDI |
| 843 | LCDR1 | SGSSSNIGSNTVN |
| 844 | LCDR2 | DNNKRPS |
| 845 | LCDR3 | SSYAGSNNVV |

Antibody 053

| | | |
|---|---|---|
| 846 | HCDR1 | SYGMH |
| 847 | HCDR2 | VTSYDGNTKYYANSVKG |
| 848 | HCDR3 | EDCGGDCFDY |
| 849 | LCDR1 | TGSSSNIGAGYDVH |
| 850 | LCDR2 | GNSNRPS |
| 851 | LCDR3 | AAWDDSLNEGV |

Antibody 054

| | | |
|---|---|---|
| 852 | HCDR1 | NYGMH |
| 853 | HCDR2 | VISYDGSNKYYADSVKG |
| 854 | HCDR3 | DQLGEAFDI |
| 855 | LCDR1 | TGSSSNIGAGYDVH |
| 856 | LCDR2 | DNNKRPS |
| 857 | LCDR3 | ATWDDSLSGPV |

Antibody 055

| | | |
|---|---|---|
| 858 | HCDR1 | DYGMS |
| 859 | HCDR2 | AISGSGSSTYYADSVKG |
| 860 | HCDR3 | GDIDYFDY |
| 861 | LCDR1 | TGSSSNFGAGYDVH |
| 862 | LCDR2 | ENNKRPS |
| 863 | LCDR3 | AAWDDSLNGPV |

Antibody 056

| | | |
|---|---|---|
| 864 | HCDR1 | SYGMH |
| 865 | HCDR2 | VISYDGSNKYYADSVKG |
| 866 | HCDR3 | ERRDAFDI |
| 867 | LCDR1 | TGSSSNIGAGYDVH |
| 868 | LCDR2 | SDNQRPS |
| 869 | LCDR3 | ATWDSDTPV |

Antibody 057

| | | |
|---|---|---|
| 870 | HCDR1 | SYGMH |
| 871 | HCDR2 | VISYDGSNKYYADSVKG |
| 872 | HCDR3 | DHSAAGYFDY |
| 873 | LCDR1 | SGSSSNIGSNTVN |
| 874 | LCDR2 | GNSIRPS |
| 875 | LCDR3 | ASWDDSLSSPV |

Antibody 058

| | | |
|---|---|---|
| 876 | HCDR1 | SYGMH |
| 877 | HCDR2 | GISWDSAIIDYAGSVKG |
| 878 | HCDR3 | DEAAAGAFDI |
| 879 | LCDR1 | TGSSSNIGAGYDVH |
| 880 | LCDR2 | GNTDRPS |
| 881 | LCDR3 | AAWDDSLSGPVV |

Antibody 059

| | | |
|---|---|---|
| 882 | HCDR1 | SYGIS |
| 883 | HCDR2 | GISGSGGNTYYADSVKG |
| 884 | HCDR3 | SVGAYANDAFDI |
| 885 | LCDR1 | TGSSSNIGAGYDVH |
| 886 | LCDR2 | GDTNRPS |
| 887 | LCDR3 | AAWDDSLNGPV |

Antibody 060

| | | |
|---|---|---|
| 888 | HCDR1 | SYGMH |
| 889 | HCDR2 | VISYDGSNKYYADSVKG |
| 890 | HCDR3 | ELYDAFDI |
| 891 | LCDR1 | TGSSSNIGAGYDVH |
| 892 | LCDR2 | ADDHRPS |
| 893 | LCDR3 | ASWDDSQRAVI |

Antibody 061

| | | |
|---|---|---|
| 894 | HCDR1 | NYGMH |
| 895 | HCDR2 | VISYDGSNKYYADSVKG |
| 896 | HCDR3 | EYKDAFDI |
| 897 | LCDR1 | TGSSSNIGSNTVN |
| 898 | LCDR2 | DNNKRPS |
| 899 | LCDR3 | QAWGTGIRV |

Antibody 062

| | | |
|---|---|---|
| 900 | HCDR1 | SYGMH |
| 901 | HCDR2 | VISYDGSNKYYADSVKG |
| 902 | HCDR3 | EFGYIILDY |
| 903 | LCDR1 | SGSSSNIGSNTVN |
| 904 | LCDR2 | RDYERPS |
| 905 | LCDR3 | MAWDDSLSGVV |

Antibody 063

| | | |
|---|---|---|
| 906 | HCDR1 | NHGMH |
| 907 | HCDR2 | VISYDGTNKYYADSVRG |
| 908 | HCDR3 | ETWDAFDV |
| 909 | LCDR1 | SGSSSNIGSNNAN |
| 910 | LCDR2 | DNNKRPS |
| 911 | LCDR3 | QAWDSSTVV |

Antibody 064

| | | |
|---|---|---|
| 912 | VH | EVKFEESGGG LVQPGGSMKLSCAASGFTFSDAWMDWVRQG |
| | | PEKGLEWVAEIRNKANNLATYYAESVKGRFTIPRDDSKSS |
| | | VYLHMNSLRAEDTGIYYCYSPFAYWGQGTLVTVSA |
| 913 | VL | DIQMTQSPSS LSASLGERVS LTCRASQEIS GYLSWLQQKP |
| | | DGTTRRLIYAASTLDSGVPK RFSGSWSGSDYSLTISSLES |
| | | EDFADYYCLQYVSYPYTFGGGTKLEIK |

Antibody 065

| | | |
|---|---|---|
| 914 | VH | QVQLVQSGAEVKKPGASVKVSCKASGYTFTNYWIHWVRQA |
| | | PGQGLEWMGVIDPSDTYPNYNKKFKGRVTMTTDTSTSTAY |
| | | MELRSLRSDDTAVYYCARNGDSDYYSGMDYWGQGTTVTVS |
| 915 | VL | EIVLTQSPDFQSVTPKEKVT ITCRTSQSIGTNIHWYQQKP |
| | | DQSPKLLIKNVSESISGVPS RFSGSGSGTDFTLTINSLEA |
| | | EDAATYYCQQSNTWPFTFGGGTKVEIK |

Antibody 066

| | | |
|---|---|---|
| 916 | VH | QVQLVQSGAEVKKPGASVKVSCKASGYTFTNYWIHWVRQA |
| | | PGQGLEWMGVIDPSDTYPNYNKKFKGRVTMTTDTSTSTAY |
| | | MELRSLRSDDTAVYYCARNGDSDYYSGMDYWGQGTTVTVS |
| 917 | VL | EIVLTQSPDF QSVTPKEKVT ITCRTSQSIG TNIHWYQQKP |
| | | DQSPKLLIKYVSESISGVPS RFSGSGSGTD FTLTINSLEA |
| | | EDAATYYCQQ SNTWPFTFGGGTKVEIK |

Antibody 067

| | | |
|---|---|---|
| 918 | VH | QVQLVQSGAEVKKPGASVKVSCKASGYTFTNYWIHWVRQA |
| | | PGQGLEWMGVIDPSDTYPNYNKKFKGRVTMTTDTSTSTAY |
| | | MELRSLRSDDTAVYYCARNGDSDYYSGMDYWGQGTTVTVS |
| 919 | VL | EIVLTQSPDF QSVTPKEKVT ITCRTSQSIG TNIHWYQQKP |
| | | DQSPKLLIKYASESISGVPS RFSGSGSGTD FTLTINSLEA |
| | | EDAATYCQQS NTWPFTFGGGTKVEIK |

CDRs

| | | |
|---|---|---|
| 920 | HCDR1 | NYWIH |
| 921 | HCDR1 | DAWMD |
| 922 | HCDR2 | VIDPSDTYPNYNKKFKG |
| 923 | HCDR2 | EIRNKANNLATYYAESVKG |
| 924 | HCDR3 | NGDSDYYSGMDY |
| 925 | HCDR3 | YSPFAY |
| 926 | LCDR1 | RTSQSIGTNIH |
| 927 | LCDR1 | RASQEISGYLS |
| 928 | LCDR2 | NVSESIS |
| 929 | LCDR2 | YVSESIS |
| 930 | LCDR2 | YASESIS |
| 931 | LCDR2 | AASTLDS |
| 932 | LCDR3 | QQSNTWPFT |
| 933 | LCDR3 | LQYVSYPYT |

In some embodiments therefore, the anti-CD32b antibodies, or antigen-binding fragment, variant, or derivative thereof, comprises at least one HCDR or LCDR identified in Table 1 or at least one HCDR or LCDR comprising at least about 70%, 75%, 80%, 85%, 90%, 95%, 96%, 97%, 98%, 99% or 100% sequence identity to a CDR sequence identified in Table 1. In some embodiments, the anti-CD32b antibodies, or antigen-binding fragment, variant, or derivative thereof, comprises two or three HCDRs and/or LCDRs identified in Table 1, or two or three HCDRs and/or LCDRs comprising at least about 70%, 75%, 80%, 85%, 90%, 95%, 96%, 97%, 98%, 99% or 100% sequence identity to a CDR sequence identified in Table 1. In some embodiments, the anti-CD32b antibodies, or antigen-binding fragment, variant, or derivative thereof, comprises one or a combination of: (a) a VH region comprising CDR1, 2 and 3 sequences comprising at least about 70%, 75%, 80%, 85%, 90%, 95%, 96%, 97%, 98%, 99% or 100% sequence identity to any of HCDR1, HCDR2 and HCDR3 disclosed in Table 1, and (b) a VL region comprising CDR1, 2 and 3 sequences comprising at least about 70%, 75%, 80%, 85%, 90%, 95%, 96%, 97%, 98%, 99% or 100% sequence identity to any of LCDR1, LCDR2 and LCDR3 disclosed in Table 1.

In some embodiments therefore, the anti-CD32b antibodies, or antigen-binding fragment, variant, or derivative thereof, comprises at least one HCDR or LCDR identified in Table 1 or at least one HCDR or LCDR comprising at least about 70%, 75%, 80%, 85%, 90%, 95%, 96%, 97%, 98%, 99% or 100% sequence identity to a CDR sequence identified as SEQ ID NO: 1 through SEQ ID NO: 756 or to a CDR sequence identified as SEQ ID NO:762 through SEQ ID NO: 933. In some embodiments, the anti-CD32b antibodies, or antigen-binding fragment, variant, or derivative thereof, comprises two or three HCDRs and/or LCDRs identified in Table 1, or two or three HCDRs and/or LCDRs comprising at least about 70%, 75%, 80%, 85%, 90%, 95%, 96%, 97%, 98%, 99% or 100% sequence identity to a CDR sequence identified as SEQ ID NO: 1 through SEQ ID NO: 756 or to a CDR sequence identified as SEQ ID NO:762 through SEQ ID NO: 933. In some embodiments, the anti-CD32b antibodies, or antigen-binding fragment, variant, or derivative thereof, comprises one or a combination of: (a) a VH region comprising CDR1, 2 and 3 sequences comprising at least about 70%, 75%, 80%, 85%, 90%, 95%, 96%, 97%, 98%, 99% or 100% sequence identity to any one or plurality of HCDR1, HCDR2 and/or HCDR3 disclosed in Table 1, and (b) a VL region comprising any one or plurality of CDR1, 2 and/or 3 sequences comprising at least about 70%, 75%, 80%, 85%, 90%, 95%, 96%, 97%, 98%, 99% or 100% sequence identity to any of LCDR1, LCDR2 and LCDR3 disclosed in Table 1.

In some embodiments, the anti-CD32b antibodies, or antigen-binding fragment, variant, or derivative thereof, comprises a VH region comprising at least about 70%, 75%, 80%, 85%, 90%, 95%, 96%, 97%, 98%, 99% or 100% sequence identity to any variable heavy sequence identified to a VH sequence identified as SEQ ID NO:1 through SEQ ID NO: 756 or to a VH sequence identified as SEQ ID NO: 762 through SEQ ID NO: 933, and a VL region comprising at least about 70%, 75%, 80%, 85%, 90%, 95%, 96%, 97%, 98%, 99% or 100% sequence identity to any variable light chain identified in Table 1, to a VL sequence identified as SEQ ID NO:1 through SEQ ID NO: 756 or to a CDR sequence identified as SEQ ID NO:762 through SEQ ID NO: 933.

In some embodiments, the methods comprise a step of treating a subject with a therapeutically effective amount of an antibody, antibody fragment or variant thereof, comprising any one or plurality of CDR sequences identified as SEQ ID NO: 1 through SEQ ID NO: 756 or to a CDR sequence identified as SEQ ID NO:762 through SEQ ID NO: 933, or comprising CDR sequences that have at least about 70%, 75%, 80%, 85%, 90%, 95%, 96%, 97%, 98%, 99% or 100% sequence identity to CDR sequences identified as SEQ ID NO:1 through SEQ ID NO: 756 or to a CDR sequence identified as SEQ ID NO:762 through SEQ ID NO: 933.

In some embodiments, the methods comprise a step of treating a subject with a therapeutically effective amount of an antibody, antibody fragment or variant thereof, comprising any one or plurality of CDR sequences identified as SEQ ID NO:1, or comprising CDR sequences that have at least about 70%, 75%, 80%, 85%, 90%, 95%, 96%, 97%, 98%, 99% or 100% sequence identity to CDR sequences identified as SEQ ID NO:1.

In some embodiments, the methods comprise a step of treating a subject with a therapeutically effective amount of an antibody, antibody fragment or variant thereof, comprising any one or plurality of CDR sequences identified as SEQ ID NO:2, or comprising CDR sequences that have at least about 70%, 75%, 80%, 85%, 90%, 95%, 96%, 97%, 98%, 99% or 100% sequence identity to CDR sequences identified as SEQ ID NO:2.

In some embodiments, the methods comprise a step of treating a subject with a therapeutically effective amount of an antibody, antibody fragment or variant thereof, comprising any one or plurality of CDR sequences identified as SEQ ID NO:3, or comprising CDR sequences that have at least about 70%, 75%, 80%, 85%, 90%, 95%, 96%, 97%, 98%, 99% or 100% sequence identity to CDR sequences identified as SEQ ID NO:3.

In some embodiments, the methods comprise a step of treating a subject with a therapeutically effective amount of an antibody, antibody fragment or variant thereof, comprising any one or plurality of CDR sequences identified as SEQ ID NO:4, or comprising CDR sequences sequence identity to CDR sequences identified as SEQ ID NO:4.

In some embodiments, the methods comprise a step of treating a subject with a therapeutically effective amount of an antibody, antibody fragment or variant thereof, comprising any one or plurality of CDR sequences identified as SEQ ID NO:5, or comprising CDR sequences that have at least about 70%, 75%, 80%, 85%, 90%, 95%, 96%, 97%, 98%, 99% or 100% sequence identity to CDR sequences identified as SEQ ID NO:5.

In some embodiments, the methods comprise a step of treating a subject with a therapeutically effective amount of an antibody, antibody fragment or variant thereof, comprising any one or plurality of CDR sequences identified as SEQ ID NO:6, or comprising CDR sequences that have at least about 70%, 75%, 80%, 85%, 90%, 95%, 96%, 97%, 98%, 99% or 100% sequence identity to CDR sequences identified as SEQ ID NO:6.

In some embodiments, the methods comprise a step of treating a subject with a therapeutically effective amount of an antibody, antibody fragment or variant thereof, comprising any one or plurality of CDR sequences identified as SEQ ID NO:7, or comprising CDR sequences that have at least about 70%, 75%, 80%, 85%, 90%, 95%, 96%, 97%, 98%, 99% or 100% sequence identity to CDR sequences identified as SEQ ID NO:7.

In some embodiments, the methods comprise a step of treating a subject with a therapeutically effective amount of an antibody, antibody fragment or variant thereof, comprising any one or plurality of CDR sequences identified as SEQ ID NO:8, or comprising CDR sequences that have at least about 70%, 75%, 80%, 85%, 90%, 95%, 96%, 97%, 98%, 99% or 100% sequence identity to CDR sequences identified as SEQ ID NO:8.

In some embodiments, the methods comprise a step of treating a subject with a therapeutically effective amount of an antibody, antibody fragment or variant thereof, comprising any one or plurality of CDR sequences identified as SEQ ID NO:9, or comprising CDR sequences that have at least about 70%, 75%, 80%, 85%, 90%, 95%, 96%, 97%, 98%, 99% or 100% sequence identity to CDR sequences identified as SEQ ID NO:9.

In some embodiments, the methods comprise a step of treating a subject with a therapeutically effective amount of an antibody, antibody fragment or variant thereof, comprising any one or plurality of CDR sequences identified as SEQ ID NO: 14, or comprising CDR sequences sequence identity to CDR sequences identified as SEQ ID NO: 14.

In some embodiments, the methods comprise a step of treating a subject with a therapeutically effective amount of an antibody, antibody fragment or variant thereof, comprising any one or plurality of CDR sequences identified as SEQ ID NO:15, or comprising CDR sequences that have at least about 70%, 75%, 80%, 85%, 90%, 95%, 96%, 97%, 98%, 99% or 100% sequence identity to CDR sequences identified as SEQ ID NO:15.

In some embodiments, the methods comprise a step of treating a subject with a therapeutically effective amount of an antibody, antibody fragment or variant thereof, comprising any one or plurality of CDR sequences identified as SEQ ID NO: 16, or comprising CDR sequences that have at least about 70%, 75%, 80%, 85%, 90%, 95%, 96%, 97%, 98%, 99% or 100% sequence identity to CDR sequences identified as SEQ ID NO:16.

In some embodiments, the methods comprise a step of treating a subject with a therapeutically effective amount of an antibody, antibody fragment or variant thereof, comprising any one or plurality of CDR sequences identified as SEQ ID NO: 17, or comprising CDR sequences that have at least about 70%, 75%, 80%, 85%, 90%, 95%, 96%, 97%, 98%, 99% or 100% sequence identity to CDR sequences identified as SEQ ID NO:17.

In some embodiments, the methods comprise a step of treating a subject with a therapeutically effective amount of an antibody, antibody fragment or variant thereof, comprising any one or plurality of CDR sequences identified as SEQ ID NO: 18, or comprising CDR sequences that have at least about 70%, 75%, 80%, 85%, 90%, 95%, 96%, 97%, 98%, 99% or 100% sequence identity to CDR sequences identified as SEQ ID NO:18.

In some embodiments, the methods comprise a step of treating a subject with a therapeutically effective amount of an antibody, antibody fragment or variant thereof, comprising any one or plurality of CDR sequences identified as SEQ ID NO:19, or comprising CDR sequences that have at least about 70%, 75%, 80%, 85%, 90%, 95%, 96%, 97%, 98%, 99% or 100% sequence identity to CDR sequences identified as SEQ ID NO:19.

In some embodiments, the methods comprise a step of treating a subject with a therapeutically effective amount of an antibody, antibody fragment or variant thereof, comprising any one or plurality of CDR sequences identified as SEQ ID NO:20, or comprising CDR sequences sequence identity to CDR sequences identified as SEQ ID NO:20.

In some embodiments, the methods comprise a step of treating a subject with a therapeutically effective amount of an antibody, antibody fragment or variant thereof, comprising any one or plurality of CDR sequences identified as SEQ ID NO:21, or comprising CDR sequences that have at least about 70%, 75%, 80%, 85%, 90%, 95%, 96%, 97%, 98%, 99% or 100% sequence identity to CDR sequences identified as SEQ ID NO:21.

In some embodiments, the methods comprise a step of treating a subject with a therapeutically effective amount of an antibody, antibody fragment or variant thereof, comprising any one or plurality of CDR sequences identified as SEQ ID NO:22, or comprising CDR sequences that have at least about 70%, 75%, 80%, 85%, 90%, 95%, 96%, 97%, 98%, 99% or 100% sequence identity to CDR sequences identified as SEQ ID NO:22.

In some embodiments, the methods comprise a step of treating a subject with a therapeutically effective amount of an antibody, antibody fragment or variant thereof, comprising any one or plurality of CDR sequences identified as SEQ ID NO:27, or comprising CDR sequences that have at least about 70%, 75%, 80%, 85%, 90%, 95%, 96%, 97%, 98%, 99% or 100% sequence identity to CDR sequences identified as SEQ ID NO:27.

In some embodiments, the methods comprise a step of treating a subject with a therapeutically effective amount of an antibody, antibody fragment or variant thereof, comprising any one or plurality of CDR sequences identified as SEQ ID NO:28, or comprising CDR sequences that have at least about 70%, 75%, 80%, 85%, 90%, 95%, 96%, 97%, 98%, 99% or 100% sequence identity to CDR sequences identified as SEQ ID NO:28.

In some embodiments, the methods comprise a step of treating a subject with a therapeutically effective amount of an antibody, antibody fragment or variant thereof, comprising any one or plurality of CDR sequences identified as SEQ ID NO:29, or comprising CDR sequences that have at least about 70%, 75%, 80%, 85%, 90%, 95%, 96%, 97%, 98%, 99% or 100% sequence identity to CDR sequences identified as SEQ ID NO:29.

In some embodiments, the methods comprise a step of treating a subject with a therapeutically effective amount of an antibody, antibody fragment or variant thereof, comprising any one or plurality of CDR sequences identified as SEQ ID NO:30, or comprising CDR sequences sequence identity to CDR sequences identified as SEQ ID NO:30.

In some embodiments, the methods comprise a step of treating a subject with a therapeutically effective amount of an antibody, antibody fragment or variant thereof, comprising any one or plurality of CDR sequences identified as SEQ ID NO:31, or comprising CDR sequences that have at least about 70%, 75%, 80%, 85%, 90%, 95%, 96%, 97%, 98%, 99% or 100% sequence identity to CDR sequences identified as SEQ ID NO:31.

In some embodiments, the methods comprise a step of treating a subject with a therapeutically effective amount of an antibody, antibody fragment or variant thereof, comprising any one or plurality of CDR sequences identified as SEQ ID NO:32, or comprising CDR sequences that have at least about 70%, 75%, 80%, 85%, 90%, 95%, 96%, 97%, 98%, 99% or 100% sequence identity to CDR sequences identified as SEQ ID NO:32.

In some embodiments, the methods comprise a step of treating a subject with a therapeutically effective amount of an antibody, antibody fragment or variant thereof, comprising any one or plurality of CDR sequences identified as SEQ ID NO:33, or comprising CDR sequences that have at least about 70%, 75%, 80%, 85%, 90%, 95%, 96%, 97%, 98%, 99% or 100% sequence identity to CDR sequences identified as SEQ ID NO:33.

In some embodiments, the methods comprise a step of treating a subject with a therapeutically effective amount of an antibody, antibody fragment or variant thereof, comprising any one or plurality of CDR sequences identified as SEQ ID NO:34, or comprising CDR sequences that have at least about 70%, 75%, 80%, 85%, 90%, 95%, 96%, 97%, 98%, 99% or 100% sequence identity to CDR sequences identified as SEQ ID NO:34.

In some embodiments, the methods comprise a step of treating a subject with a therapeutically effective amount of an antibody, antibody fragment or variant thereof, comprising any one or plurality of CDR sequences identified as SEQ ID NO:35, or comprising CDR sequences that have at least about 70%, 75%, 80%, 85%, 90%, 95%, 96%, 97%, 98%, 99% or 100% sequence identity to CDR sequences identified as SEQ ID NO:35.

In some embodiments, the methods comprise a step of treating a subject with a therapeutically effective amount of an antibody, antibody fragment or variant thereof, comprising any one or plurality of CDR sequences identified as SEQ ID NO:40, or comprising CDR sequences sequence identity to CDR sequences identified as SEQ ID NO:40.

In some embodiments, the methods comprise a step of treating a subject with a therapeutically effective amount of an antibody, antibody fragment or variant thereof, comprising any one or plurality of CDR sequences identified as SEQ ID NO:41, or comprising CDR sequences that have at least about 70%, 75%, 80%, 85%, 90%, 95%, 96%, 97%, 98%, 99% or 100% sequence identity to CDR sequences identified as SEQ ID NO:41.

In some embodiments, the methods comprise a step of treating a subject with a therapeutically effective amount of an antibody, antibody fragment or variant thereof, comprising any one or plurality of CDR sequences identified as SEQ ID NO:42, or comprising CDR sequences that have at least about 70%, 75%, 80%, 85%, 90%, 95%, 96%, 97%, 98%, 99% or 100% sequence identity to CDR sequences identified as SEQ ID NO:42.

In some embodiments, the methods comprise a step of treating a subject with a therapeutically effective amount of an antibody, antibody fragment or variant thereof, comprising any one or plurality of CDR sequences identified as SEQ ID NO:43, or comprising CDR sequences that have at least about 70%, 75%, 80%, 85%, 90%, 95%, 96%, 97%, 98%, 99% or 100% sequence identity to CDR sequences identified as SEQ ID NO:43.

In some embodiments, the methods comprise a step of treating a subject with a therapeutically effective amount of an antibody, antibody fragment or variant thereof, comprising any one or plurality of CDR sequences identified as SEQ ID NO:44, or comprising CDR sequences that have at least about 70%, 75%, 80%, 85%, 90%, 95%, 96%, 97%, 98%, 99% or 100% sequence identity to CDR sequences identified as SEQ ID NO:44.

In some embodiments, the methods comprise a step of treating a subject with a therapeutically effective amount of an antibody, antibody fragment or variant thereof, comprising any one or plurality of CDR sequences identified as SEQ ID NO:45, or comprising CDR sequences that have at least about 70%, 75%, 80%, 85%, 90%, 95%, 96%, 97%, 98%, 99% or 100% sequence identity to CDR sequences identified as SEQ ID NO:45.

In some embodiments, the methods comprise a step of treating a subject with a therapeutically effective amount of an antibody, antibody fragment or variant thereof, comprising any one or plurality of CDR sequences identified as SEQ ID NO:46, or comprising CDR sequences sequence identity to CDR sequences identified as SEQ ID NO:46.

In some embodiments, the methods comprise a step of treating a subject with a therapeutically effective amount of an antibody, antibody fragment or variant thereof, comprising any one or plurality of CDR sequences identified as SEQ ID NO:47, or comprising CDR sequences that have at least about 70%, 75%, 80%, 85%, 90%, 95%, 96%, 97%, 98%, 99% or 100% sequence identity to CDR sequences identified as SEQ ID NO:47.

In some embodiments, the methods comprise a step of treating a subject with a therapeutically effective amount of an antibody, antibody fragment or variant thereof, comprising any one or plurality of CDR sequences identified as SEQ ID NO:48, or comprising CDR sequences that have at least about 70%, 75%, 80%, 85%, 90%, 95%, 96%, 97%, 98%, 99% or 100% sequence identity to CDR sequences identified as SEQ ID NO:48.

In some embodiments, the methods comprise a step of treating a subject with a therapeutically effective amount of an antibody, antibody fragment or variant thereof, comprising any one or plurality of CDR sequences identified as SEQ ID NO:53, or comprising CDR sequences that have at least about 70%, 75%, 80%, 85%, 90%, 95%, 96%, 97%, 98%, 99% or 100% sequence identity to CDR sequences identified as SEQ ID NO:53.

In some embodiments, the methods comprise a step of treating a subject with a therapeutically effective amount of an antibody, antibody fragment or variant thereof, comprising any one or plurality of CDR sequences identified as SEQ ID NO:54, or comprising CDR sequences that have at least about 70%, 75%, 80%, 85%, 90%, 95%, 96%, 97%, 98%, 99% or 100% sequence identity to CDR sequences identified as SEQ ID NO:54.

In some embodiments, the methods comprise a step of treating a subject with a therapeutically effective amount of an antibody, antibody fragment or variant thereof, comprising any one or plurality of CDR sequences identified as SEQ ID NO:55, or comprising CDR sequences that have at least about 70%, 75%, 80%, 85%, 90%, 95%, 96%, 97%, 98%, 99% or 100% sequence identity to CDR sequences identified as SEQ ID NO:55.

In some embodiments, the methods comprise a step of treating a subject with a therapeutically effective amount of an antibody, antibody fragment or variant thereof, comprising any one or plurality of CDR sequences identified as SEQ ID NO:56, or comprising CDR sequences sequence identity to CDR sequences identified as SEQ ID NO:56.

In some embodiments, the methods comprise a step of treating a subject with a therapeutically effective amount of an antibody, antibody fragment or variant thereof, comprising any one or plurality of CDR sequences identified as SEQ ID NO:57, or comprising CDR sequences that have at least about 70%, 75%, 80%, 85%, 90%, 95%, 96%, 97%, 98%, 99% or 100% sequence identity to CDR sequences identified as SEQ ID NO:57.

In some embodiments, the methods comprise a step of treating a subject with a therapeutically effective amount of an antibody, antibody fragment or variant thereof, comprising any one or plurality of CDR sequences identified as SEQ ID NO:58, or comprising CDR sequences that have at least about 70%, 75%, 80%, 85%, 90%, 95%, 96%, 97%, 98%, 99% or 100% sequence identity to CDR sequences identified as SEQ ID NO:58.

In some embodiments, the methods comprise a step of treating a subject with a therapeutically effective amount of an antibody, antibody fragment or variant thereof, comprising any one or plurality of CDR sequences identified as SEQ ID NO:59, or comprising CDR sequences that have at least about 70%, 75%, 80%, 85%, 90%, 95%, 96%, 97%, 98%, 99% or 100% sequence identity to CDR sequences identified as SEQ ID NO:59.

In some embodiments, the methods comprise a step of treating a subject with a therapeutically effective amount of an antibody, antibody fragment or variant thereof, comprising any one or plurality of CDR sequences identified as SEQ ID NO:60, or comprising CDR sequences that have at least about 70%, 75%, 80%, 85%, 90%, 95%, 96%, 97%, 98%, 99% or 100% sequence identity to CDR sequences identified as SEQ ID NO:60.

In some embodiments, the methods comprise a step of treating a subject with a therapeutically effective amount of an antibody, antibody fragment or variant thereof, comprising any one or plurality of CDR sequences identified as SEQ ID NO:61, or comprising CDR sequences that have at least about 70%, 75%, 80%, 85%, 90%, 95%, 96%, 97%, 98%, 99% or 100% sequence identity to CDR sequences identified as SEQ ID NO:61.

In some embodiments, the methods comprise a step of treating a subject with a therapeutically effective amount of an antibody, antibody fragment or variant thereof, comprising any one or plurality of CDR sequences identified as SEQ ID NO:66, or comprising CDR sequences sequence identity to CDR sequences identified as SEQ ID NO:66.

In some embodiments, the methods comprise a step of treating a subject with a therapeutically effective amount of an antibody, antibody fragment or variant thereof, comprising any one or plurality of CDR sequences identified as SEQ ID NO:67, or comprising CDR sequences that have at least about 70%, 75%, 80%, 85%, 90%, 95%, 96%, 97%, 98%, 99% or 100% sequence identity to CDR sequences identified as SEQ ID NO:67.

In some embodiments, the methods comprise a step of treating a subject with a therapeutically effective amount of an antibody, antibody fragment or variant thereof, comprising any one or plurality of CDR sequences identified as SEQ ID NO:68, or comprising CDR sequences that have at least about 70%, 75%, 80%, 85%, 90%, 95%, 96%, 97%, 98%, 99% or 100% sequence identity to CDR sequences identified as SEQ ID NO:68.

In some embodiments, the methods comprise a step of treating a subject with a therapeutically effective amount of an antibody, antibody fragment or variant thereof, comprising any one or plurality of CDR sequences identified as SEQ ID NO:69, or comprising CDR sequences that have at least about 70%, 75%, 80%, 85%, 90%, 95%, 96%, 97%, 98%, 99% or 100% sequence identity to CDR sequences identified as SEQ ID NO:69.

In some embodiments, the methods comprise a step of treating a subject with a therapeutically effective amount of an antibody, antibody fragment or variant thereof, comprising any one or plurality of CDR sequences identified as SEQ ID NO:70, or comprising CDR sequences that have at least about 70%, 75%, 80%, 85%, 90%, 95%, 96%, 97%, 98%, 99% or 100% sequence identity to CDR sequences identified as SEQ ID NO:70.

In some embodiments, the methods comprise a step of treating a subject with a therapeutically effective amount of an antibody, antibody fragment or variant thereof, comprising any one or plurality of CDR sequences identified as SEQ ID NO:71, or comprising CDR sequences that have at least about 70%, 75%, 80%, 85%, 90%, 95%, 96%, 97%, 98%, 99% or 100% sequence identity to CDR sequences identified as SEQ ID NO:71.

In some embodiments, the methods comprise a step of treating a subject with a therapeutically effective amount of an antibody, antibody fragment or variant thereof, comprising any one or plurality of CDR sequences identified as SEQ ID NO:72, or comprising CDR sequences sequence identity to CDR sequences identified as SEQ ID NO:72.

In some embodiments, the methods comprise a step of treating a subject with a therapeutically effective amount of an antibody, antibody fragment or variant thereof, comprising any one or plurality of CDR sequences identified as SEQ ID NO:73, or comprising CDR sequences that have at least about 70%, 75%, 80%, 85%, 90%, 95%, 96%, 97%, 98%, 99% or 100% sequence identity to CDR sequences identified as SEQ ID NO:73.

In some embodiments, the methods comprise a step of treating a subject with a therapeutically effective amount of an antibody, antibody fragment or variant thereof, comprising any one or plurality of CDR sequences identified as SEQ ID NO: 74, or comprising CDR sequences that have at least about 70%, 75%, 80%, 85%, 90%, 95%, 96%, 97%, 98%, 99% or 100% sequence identity to CDR sequences identified as SEQ ID NO: 74.

In some embodiments, the methods comprise a step of treating a subject with a therapeutically effective amount of an antibody, antibody fragment or variant thereof, comprising any one or plurality of CDR sequences identified as SEQ ID NO: 79, or comprising CDR sequences that have at least about 70%, 75%, 80%, 85%, 90%, 95%, 96%, 97%, 98%, 99% or 100% sequence identity to CDR sequences identified as SEQ ID NO:79.

In some embodiments, the methods comprise a step of treating a subject with a therapeutically effective amount of an antibody, antibody fragment or variant thereof, comprising any one or plurality of CDR sequences identified as SEQ ID NO:80, or comprising CDR sequences that have at least about 70%, 75%, 80%, 85%, 90%, 95%, 96%, 97%, 98%, 99% or 100% sequence identity to CDR sequences identified as SEQ ID NO:80.

In some embodiments, the methods comprise a step of treating a subject with a therapeutically effective amount of an antibody, antibody fragment or variant thereof, comprising any one or plurality of CDR sequences identified as SEQ ID NO:81, or comprising CDR sequences that have at least about 70%, 75%, 80%, 85%, 90%, 95%, 96%, 97%, 98%, 99% or 100% sequence identity to CDR sequences identified as SEQ ID NO:81.

In some embodiments, the methods comprise a step of treating a subject with a therapeutically effective amount of an antibody, antibody fragment or variant thereof, comprising any one or plurality of CDR sequences identified as SEQ ID NO:82, or comprising CDR sequences sequence identity to CDR sequences identified as SEQ ID NO:82.

In some embodiments, the methods comprise a step of treating a subject with a therapeutically effective amount of an antibody, antibody fragment or variant thereof, comprising any one or plurality of CDR sequences identified as SEQ ID NO:83, or comprising CDR sequences that have at least about 70%, 75%, 80%, 85%, 90%, 95%, 96%, 97%, 98%, 99% or 100% sequence identity to CDR sequences identified as SEQ ID NO:83.

In some embodiments, the methods comprise a step of treating a subject with a therapeutically effective amount of an antibody, antibody fragment or variant thereof, comprising any one or plurality of CDR sequences identified as SEQ ID NO:84, or comprising CDR sequences that have at least about 70%, 75%, 80%, 85%, 90%, 95%, 96%, 97%, 98%, 99% or 100% sequence identity to CDR sequences identified as SEQ ID NO:84.

In some embodiments, the methods comprise a step of treating a subject with a therapeutically effective amount of an antibody, antibody fragment or variant thereof, comprising any one or plurality of CDR sequences identified as SEQ ID NO:85, or comprising CDR sequences that have at least about 70%, 75%, 80%, 85%, 90%, 95%, 96%, 97%, 98%, 99% or 100% sequence identity to CDR sequences identified as SEQ ID NO:85.

In some embodiments, the methods comprise a step of treating a subject with a therapeutically effective amount of an antibody, antibody fragment or variant thereof, comprising any one or plurality of CDR sequences identified as SEQ ID NO:86, or comprising CDR sequences that have at least about 70%, 75%, 80%, 85%, 90%, 95%, 96%, 97%, 98%, 99% or 100% sequence identity to CDR sequences identified as SEQ ID NO:86.

In some embodiments, the methods comprise a step of treating a subject with a therapeutically effective amount of an antibody, antibody fragment or variant thereof, comprising any one or plurality of CDR sequences identified as SEQ ID NO:87, or comprising CDR sequences that have at least about 70%, 75%, 80%, 85%, 90%, 95%, 96%, 97%, 98%, 99% or 100% sequence identity to CDR sequences identified as SEQ ID NO:87.

In some embodiments, the methods comprise a step of treating a subject with a therapeutically effective amount of an antibody, antibody fragment or variant thereof, comprising any one or plurality of CDR sequences identified as SEQ ID NO:92, or comprising CDR sequences sequence identity to CDR sequences identified as SEQ ID NO:92.

In some embodiments, the methods comprise a step of treating a subject with a therapeutically effective amount of an antibody, antibody fragment or variant thereof, comprising any one or plurality of CDR sequences identified as SEQ ID NO:93, or comprising CDR sequences that have at least about 70%, 75%, 80%, 85%, 90%, 95%, 96%, 97%, 98%, 99% or 100% sequence identity to CDR sequences identified as SEQ ID NO:93.

In some embodiments, the methods comprise a step of treating a subject with a therapeutically effective amount of an antibody, antibody fragment or variant thereof, comprising any one or plurality of CDR sequences identified as SEQ ID NO:94, or comprising CDR sequences that have at least about 70%, 75%, 80%, 85%, 90%, 95%, 96%, 97%, 98%, 99% or 100% sequence identity to CDR sequences identified as SEQ ID NO:94.

In some embodiments, the methods comprise a step of treating a subject with a therapeutically effective amount of an antibody, antibody fragment or variant thereof, comprising any one or plurality of CDR sequences identified as SEQ ID NO:95, or comprising CDR sequences that have at least about 70%, 75%, 80%, 85%, 90%, 95%, 96%, 97%, 98%, 99% or 100% sequence identity to CDR sequences identified as SEQ ID NO:95.

In some embodiments, the methods comprise a step of treating a subject with a therapeutically effective amount of an antibody, antibody fragment or variant thereof, comprising any one or plurality of CDR sequences identified as SEQ ID NO:96, or comprising CDR sequences that have at least about 70%, 75%, 80%, 85%, 90%, 95%, 96%, 97%, 98%, 99% or 100% sequence identity to CDR sequences identified as SEQ ID NO:96.

In some embodiments, the methods comprise a step of treating a subject with a therapeutically effective amount of an antibody, antibody fragment or variant thereof, comprising any one or plurality of CDR sequences identified as SEQ ID NO:97, or comprising CDR sequences that have at least about 70%, 75%, 80%, 85%, 90%, 95%, 96%, 97%, 98%, 99% or 100% sequence identity to CDR sequences identified as SEQ ID NO:97.

In some embodiments, the methods comprise a step of treating a subject with a therapeutically effective amount of an antibody, antibody fragment or variant thereof, comprising any one or plurality of CDR sequences identified as SEQ ID NO:98, or comprising CDR sequences sequence identity to CDR sequences identified as SEQ ID NO:98.

In some embodiments, the methods comprise a step of treating a subject with a therapeutically effective amount of an antibody, antibody fragment or variant thereof, comprising any one or plurality of CDR sequences identified as SEQ ID NO:99, or comprising CDR sequences that have at least about 70%, 75%, 80%, 85%, 90%, 95%, 96%, 97%, 98%, 99% or 100% sequence identity to CDR sequences identified as SEQ ID NO:99.

In some embodiments, the methods comprise a step of treating a subject with a therapeutically effective amount of an antibody, antibody fragment or variant thereof, comprising any one or plurality of CDR sequences identified as SEQ ID NO:100, or comprising CDR sequences that have at least about 70%, 75%, 80%, 85%, 90%, 95%, 96%, 97%, 98%, 99% or 100% sequence identity to CDR sequences identified as SEQ ID NO:100.

In some embodiments, the methods comprise a step of treating a subject with a therapeutically effective amount of an antibody, antibody fragment or variant thereof, comprising any one or plurality of CDR sequences identified as SEQ ID NO:105, or comprising CDR sequences that have at least about 70%, 75%, 80%, 85%, 90%, 95%, 96%, 97%, 98%, 99% or 100% sequence identity to CDR sequences identified as SEQ ID NO:105.

In some embodiments, the methods comprise a step of treating a subject with a therapeutically effective amount of an antibody, antibody fragment or variant thereof, comprising any one or plurality of CDR sequences identified as SEQ ID NO:106, or comprising CDR sequences that have at least about 70%, 75%, 80%, 85%, 90%, 95%, 96%, 97%, 98%, 99% or 100% sequence identity to CDR sequences identified as SEQ ID NO: 106.

In some embodiments, the methods comprise a step of treating a subject with a therapeutically effective amount of an antibody, antibody fragment or variant thereof, comprising any one or plurality of CDR sequences identified as SEQ ID NO:107, or comprising CDR sequences that have at least about 70%, 75%, 80%, 85%, 90%, 95%, 96%, 97%, 98%, 99% or 100% sequence identity to CDR sequences identified as SEQ ID NO:107.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:109, or comprising CDR sequences that have at least about 70%, 75%, 80%, 85%, 90%, 95%, 96%, 97%, 98%, 99% or 100% sequence identity to CDR sequences identified as SEQ ID NO:109.

In some embodiments, the methods comprise a step of treating a subject with a therapeutically effective amount of an antibody, antibody fragment or variant thereof, comprising any one or plurality of CDR sequences identified as SEQ ID NO:110, or comprising CDR sequences that have at least about 70%, 75%, 80%, 85%, 90%, 95%, 96%, 97%, 98%, 99% or 100% sequence identity to CDR sequences identified as SEQ ID NO:110.

In some embodiments, the methods comprise a step of treating a subject with a therapeutically effective amount of an antibody, antibody fragment or variant thereof, comprising any one or plurality of CDR sequences identified as SEQ ID NO:111, or comprising CDR sequences that have at least about 70%, 75%, 80%, 85%, 90%, 95%, 96%, 97%, 98%, 99% or 100% sequence identity to CDR sequences identified as SEQ ID NO:111.

In some embodiments, the methods comprise a step of treating a subject with a therapeutically effective amount of an antibody, antibody fragment or variant thereof, comprising any one or plurality of CDR sequences identified as SEQ ID NO:112, or comprising CDR sequences that have at least about 70%, 75%, 80%, 85%, 90%, 95%, 96%, 97%, 98%, 99% or 100% sequence identity to CDR sequences identified as SEQ ID NO:112.

In some embodiments, the methods comprise a step of treating a subject with a therapeutically effective amount of an antibody, antibody fragment or variant thereof, comprising any one or plurality of CDR sequences identified as SEQ ID NO:113, or comprising CDR sequences that have at least about 70%, 75%, 80%, 85%, 90%, 95%, 96%, 97%, 98%, 99% or 100% sequence identity to CDR sequences identified as SEQ ID NO:113.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:119, or comprising CDR sequences that have at least about 70%, 75%, 80%, 85%, 90%, 95%, 96%, 97%, 98%, 99% or 100% sequence identity to CDR sequences identified as SEQ ID NO:119.

In some embodiments, the methods comprise a step of treating a subject with a therapeutically effective amount of an antibody, antibody fragment or variant thereof, comprising any one or plurality of CDR sequences identified as SEQ ID NO:120, or comprising CDR sequences that have at least about 70%, 75%, 80%, 85%, 90%, 95%, 96%, 97%, 98%, 99% or 100% sequence identity to CDR sequences identified as SEQ ID NO:120.

In some embodiments, the methods comprise a step of treating a subject with a therapeutically effective amount of an antibody, antibody fragment or variant thereof, comprising any one or plurality of CDR sequences identified as SEQ ID NO:121, or comprising CDR sequences that have at least about 70%, 75%, 80%, 85%, 90%, 95%, 96%, 97%, 98%, 99% or 100% sequence identity to CDR sequences identified as SEQ ID NO:121.

In some embodiments, the methods comprise a step of treating a subject with a therapeutically effective amount of an antibody, antibody fragment or variant thereof, comprising any one or plurality of CDR sequences identified as SEQ ID NO:122, or comprising CDR sequences that have at least about 70%, 75%, 80%, 85%, 90%, 95%, 96%, 97%, 98%, 99% or 100% sequence identity to CDR sequences identified as SEQ ID NO: 122.

In some embodiments, the methods comprise a step of treating a subject with a therapeutically effective amount of an antibody, antibody fragment or variant thereof, comprising any one or plurality of CDR sequences identified as SEQ ID NO:123, or comprising CDR sequences that have at least about 70%, 75%, 80%, 85%, 90%, 95%, 96%, 97%, 98%, 99% or 100% sequence identity to CDR sequences identified as SEQ ID NO:123.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:125, or comprising CDR sequences that have at least about 70%, 75%, 80%, 85%, 90%, 95%, 96%, 97%, 98%, 99% or 100% sequence identity to CDR sequences identified as SEQ ID NO:125.

In some embodiments, the methods comprise a step of treating a subject with a therapeutically effective amount of an antibody, antibody fragment or variant thereof, comprising any one or plurality of CDR sequences identified as SEQ ID NO:126, or comprising CDR sequences that have at least about 70%, 75%, 80%, 85%, 90%, 95%, 96%, 97%, 98%, 99% or 100% sequence identity to CDR sequences identified as SEQ ID NO:126.

In some embodiments, the methods comprise a step of treating a subject with a therapeutically effective amount of an antibody, antibody fragment or variant thereof, comprising any one or plurality of CDR sequences identified as SEQ ID NO:131, or comprising CDR sequences that have at least about 70%, 75%, 80%, 85%, 90%, 95%, 96%, 97%, 98%, 99% or 100% sequence identity to CDR sequences identified as SEQ ID NO: 131.

In some embodiments, the methods comprise a step of treating a subject with a therapeutically effective amount of an antibody, antibody fragment or variant thereof, comprising any one or plurality of CDR sequences identified as SEQ ID NO:132, or comprising CDR sequences that have at least about 70%, 75%, 80%, 85%, 90%, 95%, 96%, 97%, 98%, 99% or 100% sequence identity to CDR sequences identified as SEQ ID NO: 132.

In some embodiments, the methods comprise a step of treating a subject with a therapeutically effective amount of an antibody, antibody fragment or variant thereof, comprising any one or plurality of CDR sequences identified as SEQ ID NO:133, or comprising CDR sequences that have at least about 70%, 75%, 80%, 85%, 90%, 95%, 96%, 97%, 98%, 99% or 100% sequence identity to CDR sequences identified as SEQ ID NO:133.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:135, or comprising CDR sequences that have at least about 70%, 75%, 80%, 85%, 90%, 95%, 96%, 97%, 98%, 99% or 100% sequence identity to CDR sequences identified as SEQ ID NO: 135.

In some embodiments, the methods comprise a step of treating a subject with a therapeutically effective amount of an antibody, antibody fragment or variant thereof, comprising any one or plurality of CDR sequences identified as SEQ ID NO:136, or comprising CDR sequences that have at least about 70%, 75%, 80%, 85%, 90%, 95%, 96%, 97%, 98%, 99% or 100% sequence identity to CDR sequences identified as SEQ ID NO:136.

In some embodiments, the methods comprise a step of treating a subject with a therapeutically effective amount of an antibody, antibody fragment or variant thereof, comprising any one or plurality of CDR sequences identified as SEQ ID NO:137, or comprising CDR sequences that have at least about 70%, 75%, 80%, 85%, 90%, 95%, 96%, 97%, 98%, 99% or 100% sequence identity to CDR sequences identified as SEQ ID NO: 137.

In some embodiments, the methods comprise a step of treating a subject with a therapeutically effective amount of an antibody, antibody fragment or variant thereof, comprising any one or plurality of CDR sequences identified as SEQ ID NO:138, or comprising CDR sequences that have at least about 70%, 75%, 80%, 85%, 90%, 95%, 96%, 97%, 98%, 99% or 100% sequence identity to CDR sequences identified as SEQ ID NO:138.

In some embodiments, the methods comprise a step of treating a subject with a therapeutically effective amount of an antibody, antibody fragment or variant thereof, comprising any one or plurality of CDR sequences identified as SEQ ID NO:139, or comprising CDR sequences that have at least about 70%, 75%, 80%, 85%, 90%, 95%, 96%, 97%, 98%, 99% or 100% sequence identity to CDR sequences identified as SEQ ID NO:139.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:145, or comprising CDR sequences that have at least about 70%, 75%, 80%, 85%, 90%, 95%, 96%, 97%, 98%, 99% or 100% sequence identity to CDR sequences identified as SEQ ID NO:145.

In some embodiments, the methods comprise a step of treating a subject with a therapeutically effective amount of an antibody, antibody fragment or variant thereof, comprising any one or plurality of CDR sequences identified as SEQ ID NO:146, or comprising CDR sequences that have at least about 70%, 75%, 80%, 85%, 90%, 95%, 96%, 97%, 98%, 99% or 100% sequence identity to CDR sequences identified as SEQ ID NO:146.

In some embodiments, the methods comprise a step of treating a subject with a therapeutically effective amount of an antibody, antibody fragment or variant thereof, comprising any one or plurality of CDR sequences identified as SEQ ID NO:147, or comprising CDR sequences that have at least about 70%, 75%, 80%, 85%, 90%, 95%, 96%, 97%, 98%, 99% or 100% sequence identity to CDR sequences identified as SEQ ID NO:147.

In some embodiments, the methods comprise a step of treating a subject with a therapeutically effective amount of an antibody, antibody fragment or variant thereof, comprising any one or plurality of CDR sequences identified as SEQ ID NO:148, or comprising CDR sequences that have at least about 70%, 75%, 80%, 85%, 90%, 95%, 96%, 97%, 98%, 99% or 100% sequence identity to CDR sequences identified as SEQ ID NO:148.

In some embodiments, the methods comprise a step of treating a subject with a therapeutically effective amount of an antibody, antibody fragment or variant thereof, comprising any one or plurality of CDR sequences identified as SEQ ID NO:149, or comprising CDR sequences that have at least about 70%, 75%, 80%, 85%, 90%, 95%, 96%, 97%, 98%, 99% or 100% sequence identity to CDR sequences identified as SEQ ID NO:149.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:151, or comprising CDR sequences that have at least about 70%, 75%, 80%, 85%, 90%, 95%, 96%, 97%, 98%, 99% or 100% sequence identity to CDR sequences identified as SEQ ID NO: 151.

In some embodiments, the methods comprise a step of treating a subject with a therapeutically effective amount of an antibody, antibody fragment or variant thereof, comprising any one or plurality of CDR sequences identified as SEQ ID NO:152, or comprising CDR sequences that have at least about 70%, 75%, 80%, 85%, 90%, 95%, 96%, 97%, 98%, 99% or 100% sequence identity to CDR sequences identified as SEQ ID NO: 152.

In some embodiments, the methods comprise a step of treating a subject with a therapeutically effective amount of an antibody, antibody fragment or variant thereof, comprising any one or plurality of CDR sequences identified as SEQ ID NO:157, or comprising CDR sequences that have at least about 70%, 75%, 80%, 85%, 90%, 95%, 96%, 97%, 98%, 99% or 100% sequence identity to CDR sequences identified as SEQ ID NO: 157.

In some embodiments, the methods comprise a step of treating a subject with a therapeutically effective amount of an antibody, antibody fragment or variant thereof, comprising any one or plurality of CDR sequences identified as SEQ ID NO:158, or comprising CDR sequences that have at least about 70%, 75%, 80%, 85%, 90%, 95%, 96%, 97%, 98%, 99% or 100% sequence identity to CDR sequences identified as SEQ ID NO: 158.

In some embodiments, the methods comprise a step of treating a subject with a therapeutically effective amount of an antibody, antibody fragment or variant thereof, comprising any one or plurality of CDR sequences identified as SEQ ID NO:159, or comprising CDR sequences that have at least about 70%, 75%, 80%, 85%, 90%, 95%, 96%, 97%, 98%, 99% or 100% sequence identity to CDR sequences identified as SEQ ID NO:159.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:161, or comprising CDR sequences that have at least about 70%, 75%, 80%, 85%, 90%, 95%, 96%, 97%, 98%, 99% or 100% sequence identity to CDR sequences identified as SEQ ID NO: 161.

In some embodiments, the methods comprise a step of treating a subject with a therapeutically effective amount of an antibody, antibody fragment or variant thereof, comprising any one or plurality of CDR sequences identified as SEQ ID NO:162, or comprising CDR sequences that have at least about 70%, 75%, 80%, 85%, 90%, 95%, 96%, 97%, 98%, 99% or 100% sequence identity to CDR sequences identified as SEQ ID NO: 162.

In some embodiments, the methods comprise a step of treating a subject with a therapeutically effective amount of an antibody, antibody fragment or variant thereof, comprising any one or plurality of CDR sequences identified as SEQ ID NO:163, or comprising CDR sequences that have at least about 70%, 75%, 80%, 85%, 90%, 95%, 96%, 97%, 98%, 99% or 100% sequence identity to CDR sequences identified as SEQ ID NO:163.

In some embodiments, the methods comprise a step of treating a subject with a therapeutically effective amount of an antibody, antibody fragment or variant thereof, comprising any one or plurality of CDR sequences identified as SEQ ID NO:164, or comprising CDR sequences that have at least about 70%, 75%, 80%, 85%, 90%, 95%, 96%, 97%, 98%, 99% or 100% sequence identity to CDR sequences identified as SEQ ID NO: 164.

In some embodiments, the methods comprise a step of treating a subject with a therapeutically effective amount of an antibody, antibody fragment or variant thereof, comprising any one or plurality of CDR sequences identified as SEQ ID NO:165, or comprising CDR sequences that have at least about 70%, 75%, 80%, 85%, 90%, 95%, 96%, 97%, 98%, 99% or 100% sequence identity to CDR sequences identified as SEQ ID NO: 165.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:171, or comprising CDR sequences that have at least about 70%, 75%, 80%, 85%, 90%, 95%, 96%, 97%, 98%, 99% or 100% sequence identity to CDR sequences identified as SEQ ID NO:171.

In some embodiments, the methods comprise a step of treating a subject with a therapeutically effective amount of an antibody, antibody fragment or variant thereof, comprising any one or plurality of CDR sequences identified as SEQ ID NO:172, or comprising CDR sequences that have at least about 70%, 75%, 80%, 85%, 90%, 95%, 96%, 97%, 98%, 99% or 100% sequence identity to CDR sequences identified as SEQ ID NO: 172.

In some embodiments, the methods comprise a step of treating a subject with a therapeutically effective amount of an antibody, antibody fragment or variant thereof, comprising any one or plurality of CDR sequences identified as SEQ ID NO:173, or comprising CDR sequences that have at least about 70%, 75%, 80%, 85%, 90%, 95%, 96%, 97%, 98%, 99% or 100% sequence identity to CDR sequences identified as SEQ ID NO:173.

In some embodiments, the methods comprise a step of treating a subject with a therapeutically effective amount of an antibody, antibody fragment or variant thereof, comprising any one or plurality of CDR sequences identified as SEQ ID NO:174, or comprising CDR sequences that have at least about 70%, 75%, 80%, 85%, 90%, 95%, 96%, 97%, 98%, 99% or 100% sequence identity to CDR sequences identified as SEQ ID NO: 174.

In some embodiments, the methods comprise a step of treating a subject with a therapeutically effective amount of an antibody, antibody fragment or variant thereof, comprising any one or plurality of CDR sequences identified as SEQ ID NO:175, or comprising CDR sequences that have at least about 70%, 75%, 80%, 85%, 90%, 95%, 96%, 97%, 98%, 99% or 100% sequence identity to CDR sequences identified as SEQ ID NO:175.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:177, or comprising CDR sequences that have at least about 70%, 75%, 80%, 85%, 90%, 95%, 96%, 97%, 98%, 99% or 100% sequence identity to CDR sequences identified as SEQ ID NO: 177.

In some embodiments, the methods comprise a step of treating a subject with a therapeutically effective amount of an antibody, antibody fragment or variant thereof, comprising any one or plurality of CDR sequences identified as SEQ ID NO:178, or comprising CDR sequences that have at least about 70%, 75%, 80%, 85%, 90%, 95%, 96%, 97%, 98%, 99% or 100% sequence identity to CDR sequences identified as SEQ ID NO:178.

In some embodiments, the methods comprise a step of treating a subject with a therapeutically effective amount of an antibody, antibody fragment or variant thereof, comprising any one or plurality of CDR sequences identified as SEQ ID NO:183, or comprising CDR sequences that have at least about 70%, 75%, 80%, 85%, 90%, 95%, 96%, 97%, 98%, 99% or 100% sequence identity to CDR sequences identified as SEQ ID NO: 183.

In some embodiments, the methods comprise a step of treating a subject with a therapeutically effective amount of an antibody, antibody fragment or variant thereof, comprising any one or plurality of CDR sequences identified as SEQ ID NO:184, or comprising CDR sequences that have at least about 70%, 75%, 80%, 85%, 90%, 95%, 96%, 97%, 98%, 99% or 100% sequence identity to CDR sequences identified as SEQ ID NO: 184.

In some embodiments, the methods comprise a step of treating a subject with a therapeutically effective amount of an antibody, antibody fragment or variant thereof, comprising any one or plurality of CDR sequences identified as SEQ ID NO:185, or comprising CDR sequences that have at least about 70%, 75%, 80%, 85%, 90%, 95%, 96%, 97%, 98%, 99% or 100% sequence identity to CDR sequences identified as SEQ ID NO:185.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:187, or comprising CDR sequences that have at least about 70%, 75%, 80%, 85%, 90%, 95%, 96%, 97%, 98%, 99% or 100% sequence identity to CDR sequences identified as SEQ ID NO:187.

In some embodiments, the methods comprise a step of treating a subject with a therapeutically effective amount of an antibody, antibody fragment or variant thereof, comprising any one or plurality of CDR sequences identified as SEQ ID NO:188, or comprising CDR sequences that have at least about 70%, 75%, 80%, 85%, 90%, 95%, 96%, 97%, 98%, 99% or 100% sequence identity to CDR sequences identified as SEQ ID NO:188.

In some embodiments, the methods comprise a step of treating a subject with a therapeutically effective amount of an antibody, antibody fragment or variant thereof, comprising any one or plurality of CDR sequences identified as SEQ ID NO:189, or comprising CDR sequences that have at least about 70%, 75%, 80%, 85%, 90%, 95%, 96%, 97%, 98%, 99% or 100% sequence identity to CDR sequences identified as SEQ ID NO:189.

In some embodiments, the methods comprise a step of treating a subject with a therapeutically effective amount of an antibody, antibody fragment or variant thereof, comprising any one or plurality of CDR sequences identified as SEQ ID NO:196, or comprising CDR sequences that have at least about 70%, 75%, 80%, 85%, 90%, 95%, 96%, 97%, 98%, 99% or 100% sequence identity to CDR sequences identified as SEQ ID NO:196.

In some embodiments, the methods comprise a step of treating a subject with a therapeutically effective amount of an antibody, antibody fragment or variant thereof, comprising any one or plurality of CDR sequences identified as SEQ ID NO:197, or comprising CDR sequences that have at least about 70%, 75%, 80%, 85%, 90%, 95%, 96%, 97%, 98%, 99% or 100% sequence identity to CDR sequences identified as SEQ ID NO: 197.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:199, or comprising CDR sequences that have at least about 70%, 75%, 80%, 85%, 90%, 95%, 96%, 97%, 98%, 99% or 100% sequence identity to CDR sequences identified as SEQ ID NO:199.

In some embodiments, the methods comprise a step of treating a subject with a therapeutically effective amount of an antibody, antibody fragment or variant thereof, comprising any one or plurality of CDR sequences identified as SEQ ID NO:200, or comprising CDR sequences that have at least about 70%, 75%, 80%, 85%, 90%, 95%, 96%, 97%, 98%, 99% or 100% sequence identity to CDR sequences identified as SEQ ID NO:200.

In some embodiments, the methods comprise a step of treating a subject with a therapeutically effective amount of an antibody, antibody fragment or variant thereof, comprising any one or plurality of CDR sequences identified as SEQ ID NO:201, or comprising CDR sequences that have at least about 70%, 75%, 80%, 85%, 90%, 95%, 96%, 97%, 98%, 99% or 100% sequence identity to CDR sequences identified as SEQ ID NO:201.

In some embodiments, the methods comprise a step of treating a subject with a therapeutically effective amount of an antibody, antibody fragment or variant thereof, comprising any one or plurality of CDR sequences identified as SEQ ID NO:202, or comprising CDR sequences that have at least about 70%, 75%, 80%, 85%, 90%, 95%, 96%, 97%, 98%, 99% or 100% sequence identity to CDR sequences identified as SEQ ID NO:202.

In some embodiments, the methods comprise a step of treating a subject with a therapeutically effective amount of an antibody, antibody fragment or variant thereof, comprising any one or plurality of CDR sequences identified as SEQ ID NO:203, or comprising CDR sequences that have at least about 70%, 75%, 80%, 85%, 90%, 95%, 96%, 97%, 98%, 99% or 100% sequence identity to CDR sequences identified as SEQ ID NO:203.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:209, or comprising CDR sequences that have at least about 70%, 75%, 80%, 85%, 90%, 95%, 96%, 97%, 98%, 99% or 100% sequence identity to CDR sequences identified as SEQ ID NO:209.

In some embodiments, the methods comprise a step of treating a subject with a therapeutically effective amount of an antibody, antibody fragment or variant thereof, comprising any one or plurality of CDR sequences identified as SEQ ID NO:210, or comprising CDR sequences that have at least about 70%, 75%, 80%, 85%, 90%, 95%, 96%, 97%, 98%, 99% or 100% sequence identity to CDR sequences identified as SEQ ID NO:210.

In some embodiments, the methods comprise a step of treating a subject with a therapeutically effective amount of an antibody, antibody fragment or variant thereof, comprising any one or plurality of CDR sequences identified as SEQ ID NO:211, or comprising CDR sequences that have at least about 70%, 75%, 80%, 85%, 90%, 95%, 96%, 97%, 98%, 99% or 100% sequence identity to CDR sequences identified as SEQ ID NO:211.

In some embodiments, the methods comprise a step of treating a subject with a therapeutically effective amount of an antibody, antibody fragment or variant thereof, comprising any one or plurality of CDR sequences identified as SEQ ID NO:212, or comprising CDR sequences that have at least about 70%, 75%, 80%, 85%, 90%, 95%, 96%, 97%, 98%, 99% or 100% sequence identity to CDR sequences identified as SEQ ID NO:212.

In some embodiments, the methods comprise a step of treating a subject with a therapeutically effective amount of an antibody, antibody fragment or variant thereof, comprising any one or plurality of CDR sequences identified as SEQ ID NO:213, or comprising CDR sequences that have at least about 70%, 75%, 80%, 85%, 90%, 95%, 96%, 97%, 98%, 99% or 100% sequence identity to CDR sequences identified as SEQ ID NO:213.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:215, or comprising CDR sequences that have at least about 70%, 75%, 80%, 85%, 90%, 95%, 96%, 97%, 98%, 99% or 100% sequence identity to CDR sequences identified as SEQ ID NO:215.

In some embodiments, the methods comprise a step of treating a subject with a therapeutically effective amount of an antibody, antibody fragment or variant thereof, comprising any one or plurality of CDR sequences identified as SEQ ID NO:216, or comprising CDR sequences that have at least about 70%, 75%, 80%, 85%, 90%, 95%, 96%, 97%, 98%, 99% or 100% sequence identity to CDR sequences identified as SEQ ID NO:216.

In some embodiments, the methods comprise a step of treating a subject with a therapeutically effective amount of an antibody, antibody fragment or variant thereof, comprising any one or plurality of CDR sequences identified as SEQ ID NO:217, or comprising CDR sequences that have at least about 70%, 75%, 80%, 85%, 90%, 95%, 96%, 97%, 98%, 99% or 100% sequence identity to CDR sequences identified as SEQ ID NO:217.

In some embodiments, the methods comprise a step of treating a subject with a therapeutically effective amount of an antibody, antibody fragment or variant thereof, comprising any one or plurality of CDR sequences identified as SEQ ID NO:222, or comprising CDR sequences that have at least about 70%, 75%, 80%, 85%, 90%, 95%, 96%, 97%, 98%, 99% or 100% sequence identity to CDR sequences identified as SEQ ID NO:222.

In some embodiments, the methods comprise a step of treating a subject with a therapeutically effective amount of an antibody, antibody fragment or variant thereof, comprising any one or plurality of CDR sequences identified as SEQ ID NO:223, or comprising CDR sequences that have at least about 70%, 75%, 80%, 85%, 90%, 95%, 96%, 97%, 98%, 99% or 100% sequence identity to CDR sequences identified as SEQ ID NO:223.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:225, or comprising CDR sequences that have at least about 70%, 75%, 80%, 85%, 90%, 95%, 96%, 97%, 98%, 99% or 100% sequence identity to CDR sequences identified as SEQ ID NO:225.

In some embodiments, the methods comprise a step of treating a subject with a therapeutically effective amount of an antibody, antibody fragment or variant thereof, comprising any one or plurality of CDR sequences identified as SEQ ID NO:226, or comprising CDR sequences that have at least about 70%, 75%, 80%, 85%, 90%, 95%, 96%, 97%, 98%, 99% or 100% sequence identity to CDR sequences identified as SEQ ID NO:226.

In some embodiments, the methods comprise a step of treating a subject with a therapeutically effective amount of an antibody, antibody fragment or variant thereof, comprising any one or plurality of CDR sequences identified as SEQ ID NO:227, or comprising CDR sequences that have at least about 70%, 75%, 80%, 85%, 90%, 95%, 96%, 97%, 98%, 99% or 100% sequence identity to CDR sequences identified as SEQ ID NO:227.

In some embodiments, the methods comprise a step of treating a subject with a therapeutically effective amount of an antibody, antibody fragment or variant thereof, comprising any one or plurality of CDR sequences identified as SEQ ID NO:228, or comprising CDR sequences that have at least about 70%, 75%, 80%, 85%, 90%, 95%, 96%, 97%, 98%, 99% or 100% sequence identity to CDR sequences identified as SEQ ID NO:228.

In some embodiments, the methods comprise a step of treating a subject with a therapeutically effective amount of an antibody, antibody fragment or variant thereof, comprising any one or plurality of CDR sequences identified as SEQ ID NO:229, or comprising CDR sequences that have at least about 70%, 75%, 80%, 85%, 90%, 95%, 96%, 97%, 98%, 99% or 100% sequence identity to CDR sequences identified as SEQ ID NO:229.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:235, or comprising CDR sequences that have at least about 70%, 75%, 80%, 85%, 90%, 95%, 96%, 97%, 98%, 99% or 100% sequence identity to CDR sequences identified as SEQ ID NO:235.

In some embodiments, the methods comprise a step of treating a subject with a therapeutically effective amount of an antibody, antibody fragment or variant thereof, comprising any one or plurality of CDR sequences identified as SEQ ID NO:236, or comprising CDR sequences that have at least about 70%, 75%, 80%, 85%, 90%, 95%, 96%, 97%, 98%, 99% or 100% sequence identity to CDR sequences identified as SEQ ID NO:236.

In some embodiments, the methods comprise a step of treating a subject with a therapeutically effective amount of an antibody, antibody fragment or variant thereof, comprising any one or plurality of CDR sequences identified as SEQ ID NO:237, or comprising CDR sequences that have at least about 70%, 75%, 80%, 85%, 90%, 95%, 96%, 97%, 98%, 99% or 100% sequence identity to CDR sequences identified as SEQ ID NO:237.

In some embodiments, the methods comprise a step of treating a subject with a therapeutically effective amount of an antibody, antibody fragment or variant thereof, comprising any one or plurality of CDR sequences identified as SEQ ID NO:238, or comprising CDR sequences that have at least about 70%, 75%, 80%, 85%, 90%, 95%, 96%, 97%, 98%, 99% or 100% sequence identity to CDR sequences identified as SEQ ID NO:238.

In some embodiments, the methods comprise a step of treating a subject with a therapeutically effective amount of an antibody, antibody fragment or variant thereof, comprising any one or plurality of CDR sequences identified as SEQ ID NO:239, or comprising CDR sequences that have at least about 70%, 75%, 80%, 85%, 90%, 95%, 96%, 97%, 98%, 99% or 100% sequence identity to CDR sequences identified as SEQ ID NO:239.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:241, or comprising CDR sequences that have at least about 70%, 75%, 80%, 85%, 90%, 95%, 96%, 97%, 98%, 99% or 100% sequence identity to CDR sequences identified as SEQ ID NO:241.

In some embodiments, the methods comprise a step of treating a subject with a therapeutically effective amount of an antibody, antibody fragment or variant thereof, comprising any one or plurality of CDR sequences identified as SEQ ID NO:242, or comprising CDR sequences that have at least about 70%, 75%, 80%, 85%, 90%, 95%, 96%, 97%, 98%, 99% or 100% sequence identity to CDR sequences identified as SEQ ID NO:242.

In some embodiments, the methods comprise a step of treating a subject with a therapeutically effective amount of an antibody, antibody fragment or variant thereof, comprising any one or plurality of CDR sequences identified as SEQ ID NO:243, or comprising CDR sequences that have at least about 70%, 75%, 80%, 85%, 90%, 95%, 96%, 97%, 98%, 99% or 100% sequence identity to CDR sequences identified as SEQ ID NO:243.

In some embodiments, the methods comprise a step of treating a subject with a therapeutically effective amount of an antibody, antibody fragment or variant thereof, comprising any one or plurality of CDR sequences identified as SEQ ID NO:248, or comprising CDR sequences that have at least about 70%, 75%, 80%, 85%, 90%, 95%, 96%, 97%, 98%, 99% or 100% sequence identity to CDR sequences identified as SEQ ID NO:248.

In some embodiments, the methods comprise a step of treating a subject with a therapeutically effective amount of an antibody, antibody fragment or variant thereof, comprising any one or plurality of CDR sequences identified as SEQ ID NO:249, or comprising CDR sequences that have at least about 70%, 75%, 80%, 85%, 90%, 95%, 96%, 97%, 98%, 99% or 100% sequence identity to CDR sequences identified as SEQ ID NO:249.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:251, or comprising CDR sequences that have at least about 70%, 75%, 80%, 85%, 90%, 95%, 96%, 97%, 98%, 99% or 100% sequence identity to CDR sequences identified as SEQ ID NO:251.

In some embodiments, the methods comprise a step of treating a subject with a therapeutically effective amount of an antibody, antibody fragment or variant thereof, comprising any one or plurality of CDR sequences identified as SEQ ID NO:252, or comprising CDR sequences that have at least about 70%, 75%, 80%, 85%, 90%, 95%, 96%, 97%, 98%, 99% or 100% sequence identity to CDR sequences identified as SEQ ID NO:252.

In some embodiments, the methods comprise a step of treating a subject with a therapeutically effective amount of an antibody, antibody fragment or variant thereof, comprising any one or plurality of CDR sequences identified as SEQ ID NO:253, or comprising CDR sequences that have at least about 70%, 75%, 80%, 85%, 90%, 95%, 96%, 97%, 98%, 99% or 100% sequence identity to CDR sequences identified as SEQ ID NO:253.

In some embodiments, the methods comprise a step of treating a subject with a therapeutically effective amount of an antibody, antibody fragment or variant thereof, comprising any one or plurality of CDR sequences identified as SEQ ID NO:254, or comprising CDR sequences that have at least about 70%, 75%, 80%, 85%, 90%, 95%, 96%, 97%, 98%, 99% or 100% sequence identity to CDR sequences identified as SEQ ID NO:254.

In some embodiments, the methods comprise a step of treating a subject with a therapeutically effective amount of an antibody, antibody fragment or variant thereof, comprising any one or plurality of CDR sequences identified as SEQ ID NO:255, or comprising CDR sequences that have at least about 70%, 75%, 80%, 85%, 90%, 95%, 96%, 97%, 98%, 99% or 100% sequence identity to CDR sequences identified as SEQ ID NO:255.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:261, or comprising CDR sequences that have at least about 70%, 75%, 80%, 85%, 90%, 95%, 96%, 97%, 98%, 99% or 100% sequence identity to CDR sequences identified as SEQ ID NO:261.

In some embodiments, the methods comprise a step of treating a subject with a therapeutically effective amount of an antibody, antibody fragment or variant thereof, comprising any one or plurality of CDR sequences identified as SEQ ID NO:262, or comprising CDR sequences that have at least about 70%, 75%, 80%, 85%, 90%, 95%, 96%, 97%, 98%, 99% or 100% sequence identity to CDR sequences identified as SEQ ID NO:262.

In some embodiments, the methods comprise a step of treating a subject with a therapeutically effective amount of an antibody, antibody fragment or variant thereof, comprising any one or plurality of CDR sequences identified as SEQ ID NO:263, or comprising CDR sequences that have at least about 70%, 75%, 80%, 85%, 90%, 95%, 96%, 97%, 98%, 99% or 100% sequence identity to CDR sequences identified as SEQ ID NO:263.

In some embodiments, the methods comprise a step of treating a subject with a therapeutically effective amount of an antibody, antibody fragment or variant thereof, comprising any one or plurality of CDR sequences identified as SEQ ID NO:264, or comprising CDR sequences that have at least about 70%, 75%, 80%, 85%, 90%, 95%, 96%, 97%, 98%, 99% or 100% sequence identity to CDR sequences identified as SEQ ID NO:264.

In some embodiments, the methods comprise a step of treating a subject with a therapeutically effective amount of an antibody, antibody fragment or variant thereof, comprising any one or plurality of CDR sequences identified as SEQ ID NO:265, or comprising CDR sequences that have at least about 70%, 75%, 80%, 85%, 90%, 95%, 96%, 97%, 98%, 99% or 100% sequence identity to CDR sequences identified as SEQ ID NO:265.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:267, or comprising CDR sequences that have at least about 70%, 75%, 80%, 85%, 90%, 95%, 96%, 97%, 98%, 99% or 100% sequence identity to CDR sequences identified as SEQ ID NO:267.

In some embodiments, the methods comprise a step of treating a subject with a therapeutically effective amount of an antibody, antibody fragment or variant thereof, comprising any one or plurality of CDR sequences identified as SEQ ID NO:268, or comprising CDR sequences that have at least about 70%, 75%, 80%, 85%, 90%, 95%, 96%, 97%, 98%, 99% or 100% sequence identity to CDR sequences identified as SEQ ID NO:268.

In some embodiments, the methods comprise a step of treating a subject with a therapeutically effective amount of an antibody, antibody fragment or variant thereof, comprising any one or plurality of CDR sequences identified as SEQ ID NO:269, or comprising CDR sequences that have at least about 70%, 75%, 80%, 85%, 90%, 95%, 96%, 97%, 98%, 99% or 100% sequence identity to CDR sequences identified as SEQ ID NO:269.

In some embodiments, the methods comprise a step of treating a subject with a therapeutically effective amount of an antibody, antibody fragment or variant thereof, comprising any one or plurality of CDR sequences identified as SEQ ID NO:274, or comprising CDR sequences that have at least about 70%, 75%, 80%, 85%, 90%, 95%, 96%, 97%, 98%, 99% or 100% sequence identity to CDR sequences identified as SEQ ID NO:274.

In some embodiments, the methods comprise a step of treating a subject with a therapeutically effective amount of an antibody, antibody fragment or variant thereof, comprising any one or plurality of CDR sequences identified as SEQ ID NO:275, or comprising CDR sequences that have at least about 70%, 75%, 80%, 85%, 90%, 95%, 96%, 97%, 98%, 99% or 100% sequence identity to CDR sequences identified as SEQ ID NO:275.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:277, or comprising CDR sequences that have at least about 70%, 75%, 80%, 85%, 90%, 95%, 96%, 97%, 98%, 99% or 100% sequence identity to CDR sequences identified as SEQ ID NO:277.

In some embodiments, the methods comprise a step of treating a subject with a therapeutically effective amount of an antibody, antibody fragment or variant thereof, comprising any one or plurality of CDR sequences identified as SEQ ID NO:278, or comprising CDR sequences that have at least about 70%, 75%, 80%, 85%, 90%, 95%, 96%, 97%, 98%, 99% or 100% sequence identity to CDR sequences identified as SEQ ID NO:278.

In some embodiments, the methods comprise a step of treating a subject with a therapeutically effective amount of an antibody, antibody fragment or variant thereof, comprising any one or plurality of CDR sequences identified as SEQ ID NO:279, or comprising CDR sequences that have at least about 70%, 75%, 80%, 85%, 90%, 95%, 96%, 97%, 98%, 99% or 100% sequence identity to CDR sequences identified as SEQ ID NO:279.

In some embodiments, the methods comprise a step of treating a subject with a therapeutically effective amount of an antibody, antibody fragment or variant thereof, comprising any one or plurality of CDR sequences identified as SEQ ID NO:280, or comprising CDR sequences that have at least about 70%, 75%, 80%, 85%, 90%, 95%, 96%, 97%, 98%, 99% or 100% sequence identity to CDR sequences identified as SEQ ID NO:280.

In some embodiments, the methods comprise a step of treating a subject with a therapeutically effective amount of an antibody, antibody fragment or variant thereof, comprising any one or plurality of CDR sequences identified as SEQ ID NO:281, or comprising CDR sequences that have at least about 70%, 75%, 80%, 85%, 90%, 95%, 96%, 97%, 98%, 99% or 100% sequence identity to CDR sequences identified as SEQ ID NO:281.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:287, or comprising CDR sequences that have at least about 70%, 75%, 80%, 85%, 90%, 95%, 96%, 97%, 98%, 99% or 100% sequence identity to CDR sequences identified as SEQ ID NO:287.

In some embodiments, the methods comprise a step of treating a subject with a therapeutically effective amount of an antibody, antibody fragment or variant thereof, comprising any one or plurality of CDR sequences identified as SEQ ID NO:288, or comprising CDR sequences that have at least about 70%, 75%, 80%, 85%, 90%, 95%, 96%, 97%, 98%, 99% or 100% sequence identity to CDR sequences identified as SEQ ID NO:288.

In some embodiments, the methods comprise a step of treating a subject with a therapeutically effective amount of an antibody, antibody fragment or variant thereof, comprising any one or plurality of CDR sequences identified as SEQ ID NO:289, or comprising CDR sequences that have at least about 70%, 75%, 80%, 85%, 90%, 95%, 96%, 97%, 98%, 99% or 100% sequence identity to CDR sequences identified as SEQ ID NO:289.

In some embodiments, the methods comprise a step of treating a subject with a therapeutically effective amount of an antibody, antibody fragment or variant thereof, comprising any one or plurality of CDR sequences identified as SEQ ID NO:290, or comprising CDR sequences that have at least about 70%, 75%, 80%, 85%, 90%, 95%, 96%, 97%, 98%, 99% or 100% sequence identity to CDR sequences identified as SEQ ID NO:290.

In some embodiments, the methods comprise a step of treating a subject with a therapeutically effective amount of an antibody, antibody fragment or variant thereof, comprising any one or plurality of CDR sequences identified as SEQ ID NO:291, or comprising CDR sequences that have at least about 70%, 75%, 80%, 85%, 90%, 95%, 96%, 97%, 98%, 99% or 100% sequence identity to CDR sequences identified as SEQ ID NO:291.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:293, or comprising CDR sequences that have at least about 70%, 75%, 80%, 85%, 90%, 95%, 96%, 97%, 98%, 99% or 100% sequence identity to CDR sequences identified as SEQ ID NO:293.

In some embodiments, the methods comprise a step of treating a subject with a therapeutically effective amount of an antibody, antibody fragment or variant thereof, comprising any one or plurality of CDR sequences identified as SEQ ID NO:294, or comprising CDR sequences that have at least about 70%, 75%, 80%, 85%, 90%, 95%, 96%, 97%, 98%, 99% or 100% sequence identity to CDR sequences identified as SEQ ID NO:294.

In some embodiments, the methods comprise a step of treating a subject with a therapeutically effective amount of an antibody, antibody fragment or variant thereof, comprising any one or plurality of CDR sequences identified as SEQ ID NO:295, or comprising CDR sequences that have at least about 70%, 75%, 80%, 85%, 90%, 95%, 96%, 97%, 98%, 99% or 100% sequence identity to CDR sequences identified as SEQ ID NO:295.

In some embodiments, the methods comprise a step of treating a subject with a therapeutically effective amount of an antibody, antibody fragment or variant thereof, comprising any one or plurality of CDR sequences identified as SEQ ID NO:300, or comprising CDR sequences that have at least about 70%, 75%, 80%, 85%, 90%, 95%, 96%, 97%, 98%, 99% or 100% sequence identity to CDR sequences identified as SEQ ID NO:300.

In some embodiments, the methods comprise a step of treating a subject with a therapeutically effective amount of an antibody, antibody fragment or variant thereof, comprising any one or plurality of CDR sequences identified as SEQ ID NO:301, or comprising CDR sequences that have at least about 70%, 75%, 80%, 85%, 90%, 95%, 96%, 97%, 98%, 99% or 100% sequence identity to CDR sequences identified as SEQ ID NO:301.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:303, or comprising CDR sequences that have at least about 70%, 75%, 80%, 85%, 90%, 95%, 96%, 97%, 98%, 99% or 100% sequence identity to CDR sequences identified as SEQ ID NO:303.

In some embodiments, the methods comprise a step of treating a subject with a therapeutically effective amount of an antibody, antibody fragment or variant thereof, comprising any one or plurality of CDR sequences identified as SEQ ID NO:304, or comprising CDR sequences that have at least about 70%, 75%, 80%, 85%, 90%, 95%, 96%, 97%, 98%, 99% or 100% sequence identity to CDR sequences identified as SEQ ID NO:304.

In some embodiments, the methods comprise a step of treating a subject with a therapeutically effective amount of an antibody, antibody fragment or variant thereof, comprising any one or plurality of CDR sequences identified as SEQ ID NO:305, or comprising CDR sequences that have at least about 70%, 75%, 80%, 85%, 90%, 95%, 96%, 97%, 98%, 99% or 100% sequence identity to CDR sequences identified as SEQ ID NO:305.

In some embodiments, the methods comprise a step of treating a subject with a therapeutically effective amount of an antibody, antibody fragment or variant thereof, comprising any one or plurality of CDR sequences identified as SEQ ID NO:306, or comprising CDR sequences that have at least about 70%, 75%, 80%, 85%, 90%, 95%, 96%, 97%, 98%, 99% or 100% sequence identity to CDR sequences identified as SEQ ID NO:306.

In some embodiments, the methods comprise a step of treating a subject with a therapeutically effective amount of an antibody, antibody fragment or variant thereof, comprising any one or plurality of CDR sequences identified as SEQ ID NO:307, or comprising CDR sequences that have at least about 70%, 75%, 80%, 85%, 90%, 95%, 96%, 97%, 98%, 99% or 100% sequence identity to CDR sequences identified as SEQ ID NO:307.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:313, or comprising CDR sequences that have at least about 70%, 75%, 80%, 85%, 90%, 95%, 96%, 97%, 98%, 99% or 100% sequence identity to CDR sequences identified as SEQ ID NO:313.

In some embodiments, the methods comprise a step of treating a subject with a therapeutically effective amount of an antibody, antibody fragment or variant thereof, comprising any one or plurality of CDR sequences identified as SEQ ID NO:314, or comprising CDR sequences that have at least about 70%, 75%, 80%, 85%, 90%, 95%, 96%, 97%, 98%, 99% or 100% sequence identity to CDR sequences identified as SEQ ID NO:314.

In some embodiments, the methods comprise a step of treating a subject with a therapeutically effective amount of an antibody, antibody fragment or variant thereof, comprising any one or plurality of CDR sequences identified as SEQ ID NO:315, or comprising CDR sequences that have at least about 70%, 75%, 80%, 85%, 90%, 95%, 96%, 97%, 98%, 99% or 100% sequence identity to CDR sequences identified as SEQ ID NO:315.

In some embodiments, the methods comprise a step of treating a subject with a therapeutically effective amount of an antibody, antibody fragment or variant thereof, comprising any one or plurality of CDR sequences identified as SEQ ID NO:316, or comprising CDR sequences that have at least about 70%, 75%, 80%, 85%, 90%, 95%, 96%, 97%, 98%, 99% or 100% sequence identity to CDR sequences identified as SEQ ID NO:316.

In some embodiments, the methods comprise a step of treating a subject with a therapeutically effective amount of an antibody, antibody fragment or variant thereof, comprising any one or plurality of CDR sequences identified as SEQ ID NO:317, or comprising CDR sequences that have at least about 70%, 75%, 80%, 85%, 90%, 95%, 96%, 97%, 98%, 99% or 100% sequence identity to CDR sequences identified as SEQ ID NO:317.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:319, or comprising CDR sequences that have at least about 70%, 75%, 80%, 85%, 90%, 95%, 96%, 97%, 98%, 99% or 100% sequence identity to CDR sequences identified as SEQ ID NO:319.

In some embodiments, the methods comprise a step of treating a subject with a therapeutically effective amount of an antibody, antibody fragment or variant thereof, comprising any one or plurality of CDR sequences identified as SEQ ID NO:320, or comprising CDR sequences that have at least about 70%, 75%, 80%, 85%, 90%, 95%, 96%, 97%, 98%, 99% or 100% sequence identity to CDR sequences identified as SEQ ID NO:320.

In some embodiments, the methods comprise a step of treating a subject with a therapeutically effective amount of an antibody, antibody fragment or variant thereof, comprising any one or plurality of CDR sequences identified as SEQ ID NO:321, or comprising CDR sequences that have at least about 70%, 75%, 80%, 85%, 90%, 95%, 96%, 97%, 98%, 99% or 100% sequence identity to CDR sequences identified as SEQ ID NO:321.

In some embodiments, the methods comprise a step of treating a subject with a therapeutically effective amount of an antibody, antibody fragment or variant thereof, comprising any one or plurality of CDR sequences identified as SEQ ID NO:326, or comprising CDR sequences that have at least about 70%, 75%, 80%, 85%, 90%, 95%, 96%, 97%, 98%, 99% or 100% sequence identity to CDR sequences identified as SEQ ID NO:326.

In some embodiments, the methods comprise a step of treating a subject with a therapeutically effective amount of an antibody, antibody fragment or variant thereof, comprising any one or plurality of CDR sequences identified as SEQ ID NO:327, or comprising CDR sequences that have at least about 70%, 75%, 80%, 85%, 90%, 95%, 96%, 97%, 98%, 99% or 100% sequence identity to CDR sequences identified as SEQ ID NO:327.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:329, or comprising CDR sequences that have at least about 70%, 75%, 80%, 85%, 90%, 95%, 96%, 97%, 98%, 99% or 100% sequence identity to CDR sequences identified as SEQ ID NO:329.

In some embodiments, the methods comprise a step of treating a subject with a therapeutically effective amount of an antibody, antibody fragment or variant thereof, comprising any one or plurality of CDR sequences identified as SEQ ID NO:330, or comprising CDR sequences that have at least about 70%, 75%, 80%, 85%, 90%, 95%, 96%, 97%, 98%, 99% or 100% sequence identity to CDR sequences identified as SEQ ID NO:330.

In some embodiments, the methods comprise a step of treating a subject with a therapeutically effective amount of an antibody, antibody fragment or variant thereof, comprising any one or plurality of CDR sequences identified as SEQ ID NO:331, or comprising CDR sequences that have at least about 70%, 75%, 80%, 85%, 90%, 95%, 96%, 97%, 98%, 99% or 100% sequence identity to CDR sequences identified as SEQ ID NO:331.

In some embodiments, the methods comprise a step of treating a subject with a therapeutically effective amount of an antibody, antibody fragment or variant thereof, comprising any one or plurality of CDR sequences identified as SEQ ID NO:332, or comprising CDR sequences that have at least about 70%, 75%, 80%, 85%, 90%, 95%, 96%, 97%, 98%, 99% or 100% sequence identity to CDR sequences identified as SEQ ID NO:332.

In some embodiments, the methods comprise a step of treating a subject with a therapeutically effective amount of an antibody, antibody fragment or variant thereof, comprising any one or plurality of CDR sequences identified as SEQ ID NO:333, or comprising CDR sequences that have at least about 70%, 75%, 80%, 85%, 90%, 95%, 96%, 97%, 98%, 99% or 100% sequence identity to CDR sequences identified as SEQ ID NO:333.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:339, or comprising CDR sequences that have at least about 70%, 75%, 80%, 85%, 90%, 95%, 96%, 97%, 98%, 99% or 100% sequence identity to CDR sequences identified as SEQ ID NO:339.

In some embodiments, the methods comprise a step of treating a subject with a therapeutically effective amount of an antibody, antibody fragment or variant thereof, comprising any one or plurality of CDR sequences identified as SEQ ID NO:340, or comprising CDR sequences that have at least about 70%, 75%, 80%, 85%, 90%, 95%, 96%, 97%, 98%, 99% or 100% sequence identity to CDR sequences identified as SEQ ID NO:340.

In some embodiments, the methods comprise a step of treating a subject with a therapeutically effective amount of an antibody, antibody fragment or variant thereof, comprising any one or plurality of CDR sequences identified as SEQ ID NO:341, or comprising CDR sequences that have at least about 70%, 75%, 80%, 85%, 90%, 95%, 96%, 97%, 98%, 99% or 100% sequence identity to CDR sequences identified as SEQ ID NO:341.

In some embodiments, the methods comprise a step of treating a subject with a therapeutically effective amount of an antibody, antibody fragment or variant thereof, comprising any one or plurality of CDR sequences identified as SEQ ID NO:342, or comprising CDR sequences that have at least about 70%, 75%, 80%, 85%, 90%, 95%, 96%, 97%, 98%, 99% or 100% sequence identity to CDR sequences identified as SEQ ID NO:342.

In some embodiments, the methods comprise a step of treating a subject with a therapeutically effective amount of an antibody, antibody fragment or variant thereof, comprising any one or plurality of CDR sequences identified as SEQ ID NO:343, or comprising CDR sequences that have at least about 70%, 75%, 80%, 85%, 90%, 95%, 96%, 97%, 98%, 99% or 100% sequence identity to CDR sequences identified as SEQ ID NO:343.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:345, or comprising CDR sequences that have at least about 70%, 75%, 80%, 85%, 90%, 95%, 96%, 97%, 98%, 99% or 100% sequence identity to CDR sequences identified as SEQ ID NO:345.

In some embodiments, the methods comprise a step of treating a subject with a therapeutically effective amount of an antibody, antibody fragment or variant thereof, comprising any one or plurality of CDR sequences identified as SEQ ID NO:346, or comprising CDR sequences that have at least about 70%, 75%, 80%, 85%, 90%, 95%, 96%, 97%, 98%, 99% or 100% sequence identity to CDR sequences identified as SEQ ID NO:346.

In some embodiments, the methods comprise a step of treating a subject with a therapeutically effective amount of an antibody, antibody fragment or variant thereof, comprising any one or plurality of CDR sequences identified as SEQ ID NO:347, or comprising CDR sequences that have at least about 70%, 75%, 80%, 85%, 90%, 95%, 96%, 97%, 98%, 99% or 100% sequence identity to CDR sequences identified as SEQ ID NO:347.

In some embodiments, the methods comprise a step of treating a subject with a therapeutically effective amount of an antibody, antibody fragment or variant thereof, comprising any one or plurality of CDR sequences identified as SEQ ID NO:352, or comprising CDR sequences that have at least about 70%, 75%, 80%, 85%, 90%, 95%, 96%, 97%, 98%, 99% or 100% sequence identity to CDR sequences identified as SEQ ID NO:352.

In some embodiments, the methods comprise a step of treating a subject with a therapeutically effective amount of an antibody, antibody fragment or variant thereof, comprising any one or plurality of CDR sequences identified as SEQ ID NO:353, or comprising CDR sequences that have at least about 70%, 75%, 80%, 85%, 90%, 95%, 96%, 97%, 98%, 99% or 100% sequence identity to CDR sequences identified as SEQ ID NO:353.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:355, or comprising CDR sequences that have at least about 70%, 75%, 80%, 85%, 90%, 95%, 96%, 97%, 98%, 99% or 100% sequence identity to CDR sequences identified as SEQ ID NO:355.

In some embodiments, the methods comprise a step of treating a subject with a therapeutically effective amount of an antibody, antibody fragment or variant thereof, comprising any one or plurality of CDR sequences identified as SEQ ID NO:356, or comprising CDR sequences that have at least about 70%, 75%, 80%, 85%, 90%, 95%, 96%, 97%, 98%, 99% or 100% sequence identity to CDR sequences identified as SEQ ID NO:356.

In some embodiments, the methods comprise a step of treating a subject with a therapeutically effective amount of an antibody, antibody fragment or variant thereof, comprising any one or plurality of CDR sequences identified as SEQ ID NO:357, or comprising CDR sequences that have at least about 70%, 75%, 80%, 85%, 90%, 95%, 96%, 97%, 98%, 99% or 100% sequence identity to CDR sequences identified as SEQ ID NO:357.

In some embodiments, the methods comprise a step of treating a subject with a therapeutically effective amount of an antibody, antibody fragment or variant thereof, comprising any one or plurality of CDR sequences identified as SEQ ID NO:358, or comprising CDR sequences that have at least about 70%, 75%, 80%, 85%, 90%, 95%, 96%, 97%, 98%, 99% or 100% sequence identity to CDR sequences identified as SEQ ID NO:358.

In some embodiments, the methods comprise a step of treating a subject with a therapeutically effective amount of an antibody, antibody fragment or variant thereof, comprising any one or plurality of CDR sequences identified as SEQ ID NO:359, or comprising CDR sequences that have at least about 70%, 75%, 80%, 85%, 90%, 95%, 96%, 97%, 98%, 99% or 100% sequence identity to CDR sequences identified as SEQ ID NO:359.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:365, or comprising CDR sequences that have at least about 70%, 75%, 80%, 85%, 90%, 95%, 96%, 97%, 98%, 99% or 100% sequence identity to CDR sequences identified as SEQ ID NO:365.

In some embodiments, the methods comprise a step of treating a subject with a therapeutically effective amount of an antibody, antibody fragment or variant thereof, comprising any one or plurality of CDR sequences identified as SEQ ID NO:366, or comprising CDR sequences that have at least about 70%, 75%, 80%, 85%, 90%, 95%, 96%, 97%, 98%, 99% or 100% sequence identity to CDR sequences identified as SEQ ID NO:366.

In some embodiments, the methods comprise a step of treating a subject with a therapeutically effective amount of an antibody, antibody fragment or variant thereof, comprising any one or plurality of CDR sequences identified as SEQ ID NO:367, or comprising CDR sequences that have at least about 70%, 75%, 80%, 85%, 90%, 95%, 96%, 97%, 98%, 99% or 100% sequence identity to CDR sequences identified as SEQ ID NO:367.

In some embodiments, the methods comprise a step of treating a subject with a therapeutically effective amount of an antibody, antibody fragment or variant thereof, comprising any one or plurality of CDR sequences identified as SEQ ID NO:368, or comprising CDR sequences that have at least about 70%, 75%, 80%, 85%, 90%, 95%, 96%, 97%, 98%, 99% or 100% sequence identity to CDR sequences identified as SEQ ID NO:368.

In some embodiments, the methods comprise a step of treating a subject with a therapeutically effective amount of an antibody, antibody fragment or variant thereof, comprising any one or plurality of CDR sequences identified as SEQ ID NO:369, or comprising CDR sequences that have at least about 70%, 75%, 80%, 85%, 90%, 95%, 96%, 97%, 98%, 99% or 100% sequence identity to CDR sequences identified as SEQ ID NO:369.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:371, or comprising CDR sequences that have at least about 70%, 75%, 80%, 85%, 90%, 95%, 96%, 97%, 98%, 99% or 100% sequence identity to CDR sequences identified as SEQ ID NO:371.

In some embodiments, the methods comprise a step of treating a subject with a therapeutically effective amount of an antibody, antibody fragment or variant thereof, comprising any one or plurality of CDR sequences identified as SEQ ID NO:372, or comprising CDR sequences that have at least about 70%, 75%, 80%, 85%, 90%, 95%, 96%, 97%, 98%, 99% or 100% sequence identity to CDR sequences identified as SEQ ID NO:372.

In some embodiments, the methods comprise a step of treating a subject with a therapeutically effective amount of an antibody, antibody fragment or variant thereof, comprising any one or plurality of CDR sequences identified as SEQ ID NO:373, or comprising CDR sequences that have at least about 70%, 75%, 80%, 85%, 90%, 95%, 96%, 97%, 98%, 99% or 100% sequence identity to CDR sequences identified as SEQ ID NO:373.

In some embodiments, the methods comprise a step of treating a subject with a therapeutically effective amount of an antibody, antibody fragment or variant thereof, comprising any one or plurality of CDR sequences identified as SEQ ID NO:378, or comprising CDR sequences that have at least about 70%, 75%, 80%, 85%, 90%, 95%, 96%, 97%, 98%, 99% or 100% sequence identity to CDR sequences identified as SEQ ID NO:378.

In some embodiments, the methods comprise a step of treating a subject with a therapeutically effective amount of an antibody, antibody fragment or variant thereof, comprising any one or plurality of CDR sequences identified as SEQ ID NO:379, or comprising CDR sequences that have at least about 70%, 75%, 80%, 85%, 90%, 95%, 96%, 97%, 98%, 99% or 100% sequence identity to CDR sequences identified as SEQ ID NO:379.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:381, or comprising CDR sequences that have at least about 70%, 75%, 80%, 85%, 90%, 95%, 96%, 97%, 98%, 99% or 100% sequence identity to CDR sequences identified as SEQ ID NO:381.

In some embodiments, the methods comprise a step of treating a subject with a therapeutically effective amount of an antibody, antibody fragment or variant thereof, comprising any one or plurality of CDR sequences identified as SEQ ID NO:382, or comprising CDR sequences that have at least about 70%, 75%, 80%, 85%, 90%, 95%, 96%, 97%, 98%, 99% or 100% sequence identity to CDR sequences identified as SEQ ID NO:382.

In some embodiments, the methods comprise a step of treating a subject with a therapeutically effective amount of an antibody, antibody fragment or variant thereof, comprising any one or plurality of CDR sequences identified as SEQ ID NO:383, or comprising CDR sequences that have at least about 70%, 75%, 80%, 85%, 90%, 95%, 96%, 97%, 98%, 99% or 100% sequence identity to CDR sequences identified as SEQ ID NO:383.

In some embodiments, the methods comprise a step of treating a subject with a therapeutically effective amount of an antibody, antibody fragment or variant thereof, comprising any one or plurality of CDR sequences identified as SEQ ID NO:384, or comprising CDR sequences that have at least about 70%, 75%, 80%, 85%, 90%, 95%, 96%, 97%, 98%, 99% or 100% sequence identity to CDR sequences identified as SEQ ID NO:384.

In some embodiments, the methods comprise a step of treating a subject with a therapeutically effective amount of an antibody, antibody fragment or variant thereof, comprising any one or plurality of CDR sequences identified as SEQ ID NO:385, or comprising CDR sequences that have at least about 70%, 75%, 80%, 85%, 90%, 95%, 96%, 97%, 98%, 99% or 100% sequence identity to CDR sequences identified as SEQ ID NO:385.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:391, or comprising CDR sequences that have at least about 70%, 75%, 80%, 85%, 90%, 95%, 96%, 97%, 98%, 99% or 100% sequence identity to CDR sequences identified as SEQ ID NO:391.

In some embodiments, the methods comprise a step of treating a subject with a therapeutically effective amount of an antibody, antibody fragment or variant thereof, comprising any one or plurality of CDR sequences identified as SEQ ID NO:392, or comprising CDR sequences that have at least about 70%, 75%, 80%, 85%, 90%, 95%, 96%, 97%, 98%, 99% or 100% sequence identity to CDR sequences identified as SEQ ID NO:392.

In some embodiments, the methods comprise a step of treating a subject with a therapeutically effective amount of an antibody, antibody fragment or variant thereof, comprising any one or plurality of CDR sequences identified as SEQ ID NO:393, or comprising CDR sequences that have at least about 70%, 75%, 80%, 85%, 90%, 95%, 96%, 97%, 98%, 99% or 100% sequence identity to CDR sequences identified as SEQ ID NO:393.

In some embodiments, the methods comprise a step of treating a subject with a therapeutically effective amount of an antibody, antibody fragment or variant thereof, comprising any one or plurality of CDR sequences identified as SEQ ID NO:394, or comprising CDR sequences that have at least about 70%, 75%, 80%, 85%, 90%, 95%, 96%, 97%, 98%, 99% or 100% sequence identity to CDR sequences identified as SEQ ID NO:394.

In some embodiments, the methods comprise a step of treating a subject with a therapeutically effective amount of an antibody, antibody fragment or variant thereof, comprising any one or plurality of CDR sequences identified as SEQ ID NO:395, or comprising CDR sequences that have at least about 70%, 75%, 80%, 85%, 90%, 95%, 96%, 97%, 98%, 99% or 100% sequence identity to CDR sequences identified as SEQ ID NO:395.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:397, or comprising CDR sequences that have at least about 70%, 75%, 80%, 85%, 90%, 95%, 96%, 97%, 98%, 99% or 100% sequence identity to CDR sequences identified as SEQ ID NO:397.

In some embodiments, the methods comprise a step of treating a subject with a therapeutically effective amount of an antibody, antibody fragment or variant thereof, comprising any one or plurality of CDR sequences identified as SEQ ID NO:398, or comprising CDR sequences that have at least about 70%, 75%, 80%, 85%, 90%, 95%, 96%, 97%, 98%, 99% or 100% sequence identity to CDR sequences identified as SEQ ID NO:398.

In some embodiments, the methods comprise a step of treating a subject with a therapeutically effective amount of an antibody, antibody fragment or variant thereof, comprising any one or plurality of CDR sequences identified as SEQ ID NO:399, or comprising CDR sequences that have at least about 70%, 75%, 80%, 85%, 90%, 95%, 96%, 97%, 98%, 99% or 100% sequence identity to CDR sequences identified as SEQ ID NO:399.

In some embodiments, the methods comprise a step of treating a subject with a therapeutically effective amount of an antibody, antibody fragment or variant thereof, comprising any one or plurality of CDR sequences identified as SEQ ID NO:404, or comprising CDR sequences that have at least about 70%, 75%, 80%, 85%, 90%, 95%, 96%, 97%, 98%, 99% or 100% sequence identity to CDR sequences identified as SEQ ID NO:404.

In some embodiments, the methods comprise a step of treating a subject with a therapeutically effective amount of an antibody, antibody fragment or variant thereof, comprising any one or plurality of CDR sequences identified as SEQ ID NO:405, or comprising CDR sequences that have at least about 70%, 75%, 80%, 85%, 90%, 95%, 96%, 97%, 98%, 99% or 100% sequence identity to CDR sequences identified as SEQ ID NO:405.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:407, or comprising CDR sequences that have at least about 70%, 75%, 80%, 85%, 90%, 95%, 96%, 97%, 98%, 99% or 100% sequence identity to CDR sequences identified as SEQ ID NO:407.

In some embodiments, the methods comprise a step of treating a subject with a therapeutically effective amount of an antibody, antibody fragment or variant thereof, comprising any one or plurality of CDR sequences identified as SEQ ID NO:408, or comprising CDR sequences that have at least about 70%, 75%, 80%, 85%, 90%, 95%, 96%, 97%, 98%, 99% or 100% sequence identity to CDR sequences identified as SEQ ID NO:408.

In some embodiments, the methods comprise a step of treating a subject with a therapeutically effective amount of an antibody, antibody fragment or variant thereof, comprising any one or plurality of CDR sequences identified as SEQ ID NO:409, or comprising CDR sequences that have at least about 70%, 75%, 80%, 85%, 90%, 95%, 96%, 97%, 98%, 99% or 100% sequence identity to CDR sequences identified as SEQ ID NO:409.

In some embodiments, the methods comprise a step of treating a subject with a therapeutically effective amount of an antibody, antibody fragment or variant thereof, comprising any one or plurality of CDR sequences identified as SEQ ID NO:410, or comprising CDR sequences that have at least about 70%, 75%, 80%, 85%, 90%, 95%, 96%, 97%, 98%, 99% or 100% sequence identity to CDR sequences identified as SEQ ID NO:410.

In some embodiments, the methods comprise a step of treating a subject with a therapeutically effective amount of an antibody, antibody fragment or variant thereof, comprising any one or plurality of CDR sequences identified as SEQ ID NO:411, or comprising CDR sequences that have at least about 70%, 75%, 80%, 85%, 90%, 95%, 96%, 97%, 98%, 99% or 100% sequence identity to CDR sequences identified as SEQ ID NO:411.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:417, or comprising CDR sequences that have at least about 70%, 75%, 80%, 85%, 90%, 95%, 96%, 97%, 98%, 99% or 100% sequence identity to CDR sequences identified as SEQ ID NO:417.

In some embodiments, the methods comprise a step of treating a subject with a therapeutically effective amount of an antibody, antibody fragment or variant thereof, comprising any one or plurality of CDR sequences identified as SEQ ID NO:418, or comprising CDR sequences that have at least about 70%, 75%, 80%, 85%, 90%, 95%, 96%, 97%, 98%, 99% or 100% sequence identity to CDR sequences identified as SEQ ID NO:418.

In some embodiments, the methods comprise a step of treating a subject with a therapeutically effective amount of an antibody, antibody fragment or variant thereof, comprising any one or plurality of CDR sequences identified as SEQ ID NO:419, or comprising CDR sequences that have at least about 70%, 75%, 80%, 85%, 90%, 95%, 96%, 97%, 98%, 99% or 100% sequence identity to CDR sequences identified as SEQ ID NO:419.

In some embodiments, the methods comprise a step of treating a subject with a therapeutically effective amount of an antibody, antibody fragment or variant thereof, comprising any one or plurality of CDR sequences identified as SEQ ID NO:420, or comprising CDR sequences that have at least about 70%, 75%, 80%, 85%, 90%, 95%, 96%, 97%, 98%, 99% or 100% sequence identity to CDR sequences identified as SEQ ID NO:420.

In some embodiments, the methods comprise a step of treating a subject with a therapeutically effective amount of an antibody, antibody fragment or variant thereof, comprising any one or plurality of CDR sequences identified as SEQ ID NO:421, or comprising CDR sequences that have at least about 70%, 75%, 80%, 85%, 90%, 95%, 96%, 97%, 98%, 99% or 100% sequence identity to CDR sequences identified as SEQ ID NO:421.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:423, or comprising CDR sequences that have at least about 70%, 75%, 80%, 85%, 90%, 95%, 96%, 97%, 98%, 99% or 100% sequence identity to CDR sequences identified as SEQ ID NO:423.

In some embodiments, the methods comprise a step of treating a subject with a therapeutically effective amount of an antibody, antibody fragment or variant thereof, comprising any one or plurality of CDR sequences identified as SEQ ID NO:424, or comprising CDR sequences that have at least about 70%, 75%, 80%, 85%, 90%, 95%, 96%, 97%, 98%, 99% or 100% sequence identity to CDR sequences identified as SEQ ID NO:424.

In some embodiments, the methods comprise a step of treating a subject with a therapeutically effective amount of an antibody, antibody fragment or variant thereof, comprising any one or plurality of CDR sequences identified as SEQ ID NO:425, or comprising CDR sequences that have at least about 70%, 75%, 80%, 85%, 90%, 95%, 96%, 97%, 98%, 99% or 100% sequence identity to CDR sequences identified as SEQ ID NO:425.

In some embodiments, the methods comprise a step of treating a subject with a therapeutically effective amount of an antibody, antibody fragment or variant thereof, comprising any one or plurality of CDR sequences identified as SEQ ID NO:430, or comprising CDR sequences that have at least about 70%, 75%, 80%, 85%, 90%, 95%, 96%, 97%, 98%, 99% or 100% sequence identity to CDR sequences identified as SEQ ID NO:430.

In some embodiments, the methods comprise a step of treating a subject with a therapeutically effective amount of an antibody, antibody fragment or variant thereof, comprising any one or plurality of CDR sequences identified as SEQ ID NO:431, or comprising CDR sequences that have at least about 70%, 75%, 80%, 85%, 90%, 95%, 96%, 97%, 98%, 99% or 100% sequence identity to CDR sequences identified as SEQ ID NO:431.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:433, or comprising CDR sequences that have at least about 70%, 75%, 80%, 85%, 90%, 95%, 96%, 97%, 98%, 99% or 100% sequence identity to CDR sequences identified as SEQ ID NO:433.

In some embodiments, the methods comprise a step of treating a subject with a therapeutically effective amount of an antibody, antibody fragment or variant thereof, comprising any one or plurality of CDR sequences identified as SEQ ID NO:434, or comprising CDR sequences that have at least about 70%, 75%, 80%, 85%, 90%, 95%, 96%, 97%, 98%, 99% or 100% sequence identity to CDR sequences identified as SEQ ID NO:434.

In some embodiments, the methods comprise a step of treating a subject with a therapeutically effective amount of an antibody, antibody fragment or variant thereof, comprising any one or plurality of CDR sequences identified as SEQ ID NO:435, or comprising CDR sequences that have at least about 70%, 75%, 80%, 85%, 90%, 95%, 96%, 97%, 98%, 99% or 100% sequence identity to CDR sequences identified as SEQ ID NO:435.

In some embodiments, the methods comprise a step of treating a subject with a therapeutically effective amount of an antibody, antibody fragment or variant thereof, comprising any one or plurality of CDR sequences identified as SEQ ID NO:436, or comprising CDR sequences that have at least about 70%, 75%, 80%, 85%, 90%, 95%, 96%, 97%, 98%, 99% or 100% sequence identity to CDR sequences identified as SEQ ID NO:436.

In some embodiments, the methods comprise a step of treating a subject with a therapeutically effective amount of an antibody, antibody fragment or variant thereof, comprising any one or plurality of CDR sequences identified as SEQ ID NO:437, or comprising CDR sequences that have at least about 70%, 75%, 80%, 85%, 90%, 95%, 96%, 97%, 98%, 99% or 100% sequence identity to CDR sequences identified as SEQ ID NO:437.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:443, or comprising CDR sequences that have at least about 70%, 75%, 80%, 85%, 90%, 95%, 96%, 97%, 98%, 99% or 100% sequence identity to CDR sequences identified as SEQ ID NO:443.

In some embodiments, the methods comprise a step of treating a subject with a therapeutically effective amount of an antibody, antibody fragment or variant thereof, comprising any one or plurality of CDR sequences identified as SEQ ID NO:444, or comprising CDR sequences that have at least about 70%, 75%, 80%, 85%, 90%, 95%, 96%, 97%, 98%, 99% or 100% sequence identity to CDR sequences identified as SEQ ID NO:444.

In some embodiments, the methods comprise a step of treating a subject with a therapeutically effective amount of an antibody, antibody fragment or variant thereof, comprising any one or plurality of CDR sequences identified as SEQ ID NO:444, or comprising CDR sequences that have at least about 70%, 75%, 80%, 85%, 90%, 95%, 96%, 97%, 98%, 99% or 100% sequence identity to CDR sequences identified as SEQ ID NO:444.

In some embodiments, the methods comprise a step of treating a subject with a therapeutically effective amount of an antibody, antibody fragment or variant thereof, comprising any one or plurality of CDR sequences identified as SEQ ID NO:445, or comprising CDR sequences that have at least about 70%, 75%, 80%, 85%, 90%, 95%, 96%, 97%, 98%, 99% or 100% sequence identity to CDR sequences identified as SEQ ID NO:445.

In some embodiments, the methods comprise a step of treating a subject with a therapeutically effective amount of an antibody, antibody fragment or variant thereof, comprising any one or plurality of CDR sequences identified as SEQ ID NO:446, or comprising CDR sequences that have at least about 70%, 75%, 80%, 85%, 90%, 95%, 96%, 97%, 98%, 99% or 100% sequence identity to CDR sequences identified as SEQ ID NO:446.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:448, or comprising CDR sequences that have at least about 70%, 75%, 80%, 85%, 90%, 95%, 96%, 97%, 98%, 99% or 100% sequence identity to CDR sequences identified as SEQ ID NO:448.

In some embodiments, the methods comprise a step of treating a subject with a therapeutically effective amount of an antibody, antibody fragment or variant thereof, comprising any one or plurality of CDR sequences identified as SEQ ID NO:449, or comprising CDR sequences that have at least about 70%, 75%, 80%, 85%, 90%, 95%, 96%, 97%, 98%, 99% or 100% sequence identity to CDR sequences identified as SEQ ID NO:449.

In some embodiments, the methods comprise a step of treating a subject with a therapeutically effective amount of an antibody, antibody fragment or variant thereof, comprising any one or plurality of CDR sequences identified as SEQ ID NO:450, or comprising CDR sequences that have at least about 70%, 75%, 80%, 85%, 90%, 95%, 96%, 97%, 98%, 99% or 100% sequence identity to CDR sequences identified as SEQ ID NO:450.

In some embodiments, the methods comprise a step of treating a subject with a therapeutically effective amount of an antibody, antibody fragment or variant thereof, comprising any one or plurality of CDR sequences identified as SEQ ID NO:451, or comprising CDR sequences that have at least about 70%, 75%, 80%, 85%, 90%, 95%, 96%, 97%, 98%, 99% or 100% sequence identity to CDR sequences identified as SEQ ID NO:451.

In some embodiments, the methods comprise a step of treating a subject with a therapeutically effective amount of an antibody, antibody fragment or variant thereof, comprising any one or plurality of CDR sequences identified as SEQ ID NO:456, or comprising CDR sequences that have at least about 70%, 75%, 80%, 85%, 90%, 95%, 96%, 97%, 98%, 99% or 100% sequence identity to CDR sequences identified as SEQ ID NO:456.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:458, or comprising CDR sequences that have at least about 70%, 75%, 80%, 85%, 90%, 95%, 96%, 97%, 98%, 99% or 100% sequence identity to CDR sequences identified as SEQ ID NO:458.

In some embodiments, the methods comprise a step of treating a subject with a therapeutically effective amount of an antibody, antibody fragment or variant thereof, comprising any one or plurality of CDR sequences identified as SEQ ID NO:459, or comprising CDR sequences that have at least about 70%, 75%, 80%, 85%, 90%, 95%, 96%, 97%, 98%, 99% or 100% sequence identity to CDR sequences identified as SEQ ID NO:459.

In some embodiments, the methods comprise a step of treating a subject with a therapeutically effective amount of an antibody, antibody fragment or variant thereof, comprising any one or plurality of CDR sequences identified as SEQ ID NO:460, or comprising CDR sequences that have at least about 70%, 75%, 80%, 85%, 90%, 95%, 96%, 97%, 98%, 99% or 100% sequence identity to CDR sequences identified as SEQ ID NO:460.

In some embodiments, the methods comprise a step of treating a subject with a therapeutically effective amount of an antibody, antibody fragment or variant thereof, comprising any one or plurality of CDR sequences identified as SEQ ID NO:461, or comprising CDR sequences that have at least about 70%, 75%, 80%, 85%, 90%, 95%, 96%, 97%, 98%, 99% or 100% sequence identity to CDR sequences identified as SEQ ID NO:461.

In some embodiments, the methods comprise a step of treating a subject with a therapeutically effective amount of an antibody, antibody fragment or variant thereof, comprising any one or plurality of CDR sequences identified as SEQ ID NO:462, or comprising CDR sequences that have at least about 70%, 75%, 80%, 85%, 90%, 95%, 96%, 97%, 98%, 99% or 100% sequence identity to CDR sequences identified as SEQ ID NO:462.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:464, or comprising CDR sequences that have at least about 70%, 75%, 80%, 85%, 90%, 95%, 96%, 97%, 98%, 99% or 100% sequence identity to CDR sequences identified as SEQ ID NO:464.

In some embodiments, the methods comprise a step of treating a subject with a therapeutically effective amount of an antibody, antibody fragment or variant thereof, comprising any one or plurality of CDR sequences identified as SEQ ID NO:469, or comprising CDR sequences that have at least about 70%, 75%, 80%, 85%, 90%, 95%, 96%, 97%, 98%, 99% or 100% sequence identity to CDR sequences identified as SEQ ID NO:469.

In some embodiments, the methods comprise a step of treating a subject with a therapeutically effective amount of an antibody, antibody fragment or variant thereof, comprising any one or plurality of CDR sequences identified as SEQ ID NO:470, or comprising CDR sequences that have at least about 70%, 75%, 80%, 85%, 90%, 95%, 96%, 97%, 98%, 99% or 100% sequence identity to CDR sequences identified as SEQ ID NO:470.

In some embodiments, the methods comprise a step of treating a subject with a therapeutically effective amount of an antibody, antibody fragment or variant thereof, comprising any one or plurality of CDR sequences identified as SEQ ID NO:471, or comprising CDR sequences that have at least about 70%, 75%, 80%, 85%, 90%, 95%, 96%, 97%, 98%, 99% or 100% sequence identity to CDR sequences identified as SEQ ID NO:471.

In some embodiments, the methods comprise a step of treating a subject with a therapeutically effective amount of an antibody, antibody fragment or variant thereof, comprising any one or plurality of CDR sequences identified as SEQ ID NO:472, or comprising CDR sequences that have at least about 70%, 75%, 80%, 85%, 90%, 95%, 96%, 97%, 98%, 99% or 100% sequence identity to CDR sequences identified as SEQ ID NO:472.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:474, or comprising CDR sequences that have at least about 70%, 75%, 80%, 85%, 90%, 95%, 96%, 97%, 98%, 99% or 100% sequence identity to CDR sequences identified as SEQ ID NO:474.

In some embodiments, the methods comprise a step of treating a subject with a therapeutically effective amount of an antibody, antibody fragment or variant thereof, comprising any one or plurality of CDR sequences identified as SEQ ID NO:475, or comprising CDR sequences that have at least about 70%, 75%, 80%, 85%, 90%, 95%, 96%, 97%, 98%, 99% or 100% sequence identity to CDR sequences identified as SEQ ID NO:475.

In some embodiments, the methods comprise a step of treating a subject with a therapeutically effective amount of an antibody, antibody fragment or variant thereof, comprising any one or plurality of CDR sequences identified as SEQ ID NO:476, or comprising CDR sequences that have at least about 70%, 75%, 80%, 85%, 90%, 95%, 96%, 97%, 98%, 99% or 100% sequence identity to CDR sequences identified as SEQ ID NO:476.

In some embodiments, the methods comprise a step of treating a subject with a therapeutically effective amount of an antibody, antibody fragment or variant thereof, comprising any one or plurality of CDR sequences identified as SEQ ID NO:477, or comprising CDR sequences that have at least about 70%, 75%, 80%, 85%, 90%, 95%, 96%, 97%, 98%, 99% or 100% sequence identity to CDR sequences identified as SEQ ID NO:477.

In some embodiments, the methods comprise a step of treating a subject with a therapeutically effective amount of an antibody, antibody fragment or variant thereof, comprising any one or plurality of CDR sequences identified as SEQ ID NO:482, or comprising CDR sequences that have at least about 70%, 75%, 80%, 85%, 90%, 95%, 96%, 97%, 98%, 99% or 100% sequence identity to CDR sequences identified as SEQ ID NO:482.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:484, or comprising CDR sequences that have at least about 70%, 75%, 80%, 85%, 90%, 95%, 96%, 97%, 98%, 99% or 100% sequence identity to CDR sequences identified as SEQ ID NO:484.

In some embodiments, the methods comprise a step of treating a subject with a therapeutically effective amount of an antibody, antibody fragment or variant thereof, comprising any one or plurality of CDR sequences identified as SEQ ID NO:485, or comprising CDR sequences that have at least about 70%, 75%, 80%, 85%, 90%, 95%, 96%, 97%, 98%, 99% or 100% sequence identity to CDR sequences identified as SEQ ID NO:485.

In some embodiments, the methods comprise a step of treating a subject with a therapeutically effective amount of an antibody, antibody fragment or variant thereof, comprising any one or plurality of CDR sequences identified as SEQ ID NO:486, or comprising CDR sequences that have at least about 70%, 75%, 80%, 85%, 90%, 95%, 96%, 97%, 98%, 99% or 100% sequence identity to CDR sequences identified as SEQ ID NO:486.

In some embodiments, the methods comprise a step of treating a subject with a therapeutically effective amount of an antibody, antibody fragment or variant thereof, comprising any one or plurality of CDR sequences identified as SEQ ID NO:487, or comprising CDR sequences that have at least about 70%, 75%, 80%, 85%, 90%, 95%, 96%, 97%, 98%, 99% or 100% sequence identity to CDR sequences identified as SEQ ID NO:487.

In some embodiments, the methods comprise a step of treating a subject with a therapeutically effective amount of an antibody, antibody fragment or variant thereof, comprising any one or plurality of CDR sequences identified as SEQ ID NO:488, or comprising CDR sequences that have at least about 70%, 75%, 80%, 85%, 90%, 95%, 96%, 97%, 98%, 99% or 100% sequence identity to CDR sequences identified as SEQ ID NO:488.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:490, or comprising CDR sequences that have at least about 70%, 75%, 80%, 85%, 90%, 95%, 96%, 97%, 98%, 99% or 100% sequence identity to CDR sequences identified as SEQ ID NO:490.

In some embodiments, the methods comprise a step of treating a subject with a therapeutically effective amount of an antibody, antibody fragment or variant thereof, comprising any one or plurality of CDR sequences identified as SEQ ID NO:495, or comprising CDR sequences that have at least about 70%, 75%, 80%, 85%, 90%, 95%, 96%, 97%, 98%, 99% or 100% sequence identity to CDR sequences identified as SEQ ID NO:495.

In some embodiments, the methods comprise a step of treating a subject with a therapeutically effective amount of an antibody, antibody fragment or variant thereof, comprising any one or plurality of CDR sequences identified as SEQ ID NO:496, or comprising CDR sequences that have at least about 70%, 75%, 80%, 85%, 90%, 95%, 96%, 97%, 98%, 99% or 100% sequence identity to CDR sequences identified as SEQ ID NO:496.

In some embodiments, the methods comprise a step of treating a subject with a therapeutically effective amount of an antibody, antibody fragment or variant thereof, comprising any one or plurality of CDR sequences identified as SEQ ID NO:497, or comprising CDR sequences that have at least about 70%, 75%, 80%, 85%, 90%, 95%, 96%, 97%, 98%, 99% or 100% sequence identity to CDR sequences identified as SEQ ID NO:497.

In some embodiments, the methods comprise a step of treating a subject with a therapeutically effective amount of an antibody, antibody fragment or variant thereof, comprising any one or plurality of CDR sequences identified as SEQ ID NO:498, or comprising CDR sequences that have at least about 70%, 75%, 80%, 85%, 90%, 95%, 96%, 97%, 98%, 99% or 100% sequence identity to CDR sequences identified as SEQ ID NO:498.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:500, or comprising CDR sequences that have at least about 70%, 75%, 80%, 85%, 90%, 95%, 96%, 97%, 98%, 99% or 100% sequence identity to CDR sequences identified as SEQ ID NO:500.

In some embodiments, the methods comprise a step of treating a subject with a therapeutically effective amount of an antibody, antibody fragment or variant thereof, comprising any one or plurality of CDR sequences identified as SEQ ID NO:501, or comprising CDR sequences that have at least about 70%, 75%, 80%, 85%, 90%, 95%, 96%, 97%, 98%, 99% or 100% sequence identity to CDR sequences identified as SEQ ID NO:501.

In some embodiments, the methods comprise a step of treating a subject with a therapeutically effective amount of an antibody, antibody fragment or variant thereof, comprising any one or plurality of CDR sequences identified as SEQ ID NO:502, or comprising CDR sequences that have at least about 70%, 75%, 80%, 85%, 90%, 95%, 96%, 97%, 98%, 99% or 100% sequence identity to CDR sequences identified as SEQ ID NO:502.

In some embodiments, the methods comprise a step of treating a subject with a therapeutically effective amount of an antibody, antibody fragment or variant thereof, comprising any one or plurality of CDR sequences identified as SEQ ID NO:503, or comprising CDR sequences that have at least about 70%, 75%, 80%, 85%, 90%, 95%, 96%, 97%, 98%, 99% or 100% sequence identity to CDR sequences identified as SEQ ID NO:503.

In some embodiments, the methods comprise a step of treating a subject with a therapeutically effective amount of an antibody, antibody fragment or variant thereof, comprising any one or plurality of CDR sequences identified as SEQ ID NO:508, or comprising CDR sequences that have at least about 70%, 75%, 80%, 85%, 90%, 95%, 96%, 97%, 98%, 99% or 100% sequence identity to CDR sequences identified as SEQ ID NO:508.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:510, or comprising CDR sequences that have at least about 70%, 75%, 80%, 85%, 90%, 95%, 96%, 97%, 98%, 99% or 100% sequence identity to CDR sequences identified as SEQ ID NO:510.

In some embodiments, the methods comprise a step of treating a subject with a therapeutically effective amount of an antibody, antibody fragment or variant thereof, comprising any one or plurality of CDR sequences identified as SEQ ID NO:511, or comprising CDR sequences that have at least about 70%, 75%, 80%, 85%, 90%, 95%, 96%, 97%, 98%, 99% or 100% sequence identity to CDR sequences identified as SEQ ID NO:511.

In some embodiments, the methods comprise a step of treating a subject with a therapeutically effective amount of an antibody, antibody fragment or variant thereof, comprising any one or plurality of CDR sequences identified as SEQ ID NO:512, or comprising CDR sequences that have at least about 70%, 75%, 80%, 85%, 90%, 95%, 96%, 97%, 98%, 99% or 100% sequence identity to CDR sequences identified as SEQ ID NO:512.

In some embodiments, the methods comprise a step of treating a subject with a therapeutically effective amount of an antibody, antibody fragment or variant thereof, comprising any one or plurality of CDR sequences identified as SEQ ID NO:513, or comprising CDR sequences that have at least about 70%, 75%, 80%, 85%, 90%, 95%, 96%, 97%, 98%, 99% or 100% sequence identity to CDR sequences identified as SEQ ID NO:513.

In some embodiments, the methods comprise a step of treating a subject with a therapeutically effective amount of an antibody, antibody fragment or variant thereof, comprising any one or plurality of CDR sequences identified as SEQ ID NO:514, or comprising CDR sequences that have at least about 70%, 75%, 80%, 85%, 90%, 95%, 96%, 97%, 98%, 99% or 100% sequence identity to CDR sequences identified as SEQ ID NO:514.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:516, or comprising CDR sequences that have at least about 70%, 75%, 80%, 85%, 90%, 95%, 96%, 97%, 98%, 99% or 100% sequence identity to CDR sequences identified as SEQ ID NO:516.

In some embodiments, the methods comprise a step of treating a subject with a therapeutically effective amount of an antibody, antibody fragment or variant thereof, comprising any one or plurality of CDR sequences identified as SEQ ID NO:521, or comprising CDR sequences that have at least about 70%, 75%, 80%, 85%, 90%, 95%, 96%, 97%, 98%, 99% or 100% sequence identity to CDR sequences identified as SEQ ID NO:521.

In some embodiments, the methods comprise a step of treating a subject with a therapeutically effective amount of an antibody, antibody fragment or variant thereof, comprising any one or plurality of CDR sequences identified as SEQ ID NO:522, or comprising CDR sequences that have at least about 70%, 75%, 80%, 85%, 90%, 95%, 96%, 97%, 98%, 99% or 100% sequence identity to CDR sequences identified as SEQ ID NO:522.

In some embodiments, the methods comprise a step of treating a subject with a therapeutically effective amount of an antibody, antibody fragment or variant thereof, comprising any one or plurality of CDR sequences identified as SEQ ID NO:523, or comprising CDR sequences that have at least about 70%, 75%, 80%, 85%, 90%, 95%, 96%, 97%, 98%, 99% or 100% sequence identity to CDR sequences identified as SEQ ID NO:523.

In some embodiments, the methods comprise a step of treating a subject with a therapeutically effective amount of an antibody, antibody fragment or variant thereof, comprising any one or plurality of CDR sequences identified as SEQ ID NO:524, or comprising CDR sequences that have at least about 70%, 75%, 80%, 85%, 90%, 95%, 96%, 97%, 98%, 99% or 100% sequence identity to CDR sequences identified as SEQ ID NO:524.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:526, or comprising CDR sequences that have at least about 70%, 75%, 80%, 85%, 90%, 95%, 96%, 97%, 98%, 99% or 100% sequence identity to CDR sequences identified as SEQ ID NO:526.

In some embodiments, the methods comprise a step of treating a subject with a therapeutically effective amount of an antibody, antibody fragment or variant thereof, comprising any one or plurality of CDR sequences identified as SEQ ID NO:527, or comprising CDR sequences that have at least about 70%, 75%, 80%, 85%, 90%, 95%, 96%, 97%, 98%, 99% or 100% sequence identity to CDR sequences identified as SEQ ID NO:527.

In some embodiments, the methods comprise a step of treating a subject with a therapeutically effective amount of an antibody, antibody fragment or variant thereof, comprising any one or plurality of CDR sequences identified as SEQ ID NO:528, or comprising CDR sequences that have at least about 70%, 75%, 80%, 85%, 90%, 95%, 96%, 97%, 98%, 99% or 100% sequence identity to CDR sequences identified as SEQ ID NO:528.

In some embodiments, the methods comprise a step of treating a subject with a therapeutically effective amount of an antibody, antibody fragment or variant thereof, comprising any one or plurality of CDR sequences identified as SEQ ID NO:529, or comprising CDR sequences that have at least about 70%, 75%, 80%, 85%, 90%, 95%, 96%, 97%, 98%, 99% or 100% sequence identity to CDR sequences identified as SEQ ID NO:529.

In some embodiments, the methods comprise a step of treating a subject with a therapeutically effective amount of an antibody, antibody fragment or variant thereof, comprising any one or plurality of CDR sequences identified as SEQ ID NO:530, or comprising CDR sequences that have at least about 70%, 75%, 80%, 85%, 90%, 95%, 96%, 97%, 98%, 99% or 100% sequence identity to CDR sequences identified as SEQ ID NO:530.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:535, or comprising CDR sequences that have at least about 70%, 75%, 80%, 85%, 90%, 95%, 96%, 97%, 98%, 99% or 100% sequence identity to CDR sequences identified as SEQ ID NO:535.

In some embodiments, the methods comprise a step of treating a subject with a therapeutically effective amount of an antibody, antibody fragment or variant thereof, comprising any one or plurality of CDR sequences identified as SEQ ID NO:536, or comprising CDR sequences that have at least about 70%, 75%, 80%, 85%, 90%, 95%, 96%, 97%, 98%, 99% or 100% sequence identity to CDR sequences identified as SEQ ID NO:536.

In some embodiments, the methods comprise a step of treating a subject with a therapeutically effective amount of an antibody, antibody fragment or variant thereof, comprising any one or plurality of CDR sequences identified as SEQ ID NO:537, or comprising CDR sequences that have at least about 70%, 75%, 80%, 85%, 90%, 95%, 96%, 97%, 98%, 99% or 100% sequence identity to CDR sequences identified as SEQ ID NO:537.

In some embodiments, the methods comprise a step of treating a subject with a therapeutically effective amount of an antibody, antibody fragment or variant thereof, comprising any one or plurality of CDR sequences identified as SEQ ID NO:538, or comprising CDR sequences that have at least about 70%, 75%, 80%, 85%, 90%, 95%, 96%, 97%, 98%, 99% or 100% sequence identity to CDR sequences identified as SEQ ID NO:538.

In some embodiments, the methods comprise a step of treating a subject with a therapeutically effective amount of an antibody, antibody fragment or variant thereof, comprising any one or plurality of CDR sequences identified as SEQ ID NO:539, or comprising CDR sequences that have at least about 70%, 75%, 80%, 85%, 90%, 95%, 96%, 97%, 98%, 99% or 100% sequence identity to CDR sequences identified as SEQ ID NO:539.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:541, or comprising CDR sequences that have at least about 70%, 75%, 80%, 85%, 90%, 95%, 96%, 97%, 98%, 99% or 100% sequence identity to CDR sequences identified as SEQ ID NO:541.

In some embodiments, the methods comprise a step of treating a subject with a therapeutically effective amount of an antibody, antibody fragment or variant thereof, comprising any one or plurality of CDR sequences identified as SEQ ID NO:542, or comprising CDR sequences that have at least about 70%, 75%, 80%, 85%, 90%, 95%, 96%, 97%, 98%, 99% or 100% sequence identity to CDR sequences identified as SEQ ID NO:542.

In some embodiments, the methods comprise a step of treating a subject with a therapeutically effective amount of an antibody, antibody fragment or variant thereof, comprising any one or plurality of CDR sequences identified as SEQ ID NO:547, or comprising CDR sequences that have at least about 70%, 75%, 80%, 85%, 90%, 95%, 96%, 97%, 98%, 99% or 100% sequence identity to CDR sequences identified as SEQ ID NO:547.

In some embodiments, the methods comprise a step of treating a subject with a therapeutically effective amount of an antibody, antibody fragment or variant thereof, comprising any one or plurality of CDR sequences identified as SEQ ID NO:548, or comprising CDR sequences that have at least about 70%, 75%, 80%, 85%, 90%, 95%, 96%, 97%, 98%, 99% or 100% sequence identity to CDR sequences identified as SEQ ID NO:548.

In some embodiments, the methods comprise a step of treating a subject with a therapeutically effective amount of an antibody, antibody fragment or variant thereof, comprising any one or plurality of CDR sequences identified as SEQ ID NO:549, or comprising CDR sequences that have at least about 70%, 75%, 80%, 85%, 90%, 95%, 96%, 97%, 98%, 99% or 100% sequence identity to CDR sequences identified as SEQ ID NO:549.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:551, or comprising CDR sequences that have at least about 70%, 75%, 80%, 85%, 90%, 95%, 96%, 97%, 98%, 99% or 100% sequence identity to CDR sequences identified as SEQ ID NO:551.

In some embodiments, the methods comprise a step of treating a subject with a therapeutically effective amount of an antibody, antibody fragment or variant thereof, comprising any one or plurality of CDR sequences identified as SEQ ID NO:552, or comprising CDR sequences that have at least about 70%, 75%, 80%, 85%, 90%, 95%, 96%, 97%, 98%, 99% or 100% sequence identity to CDR sequences identified as SEQ ID NO:552.

In some embodiments, the methods comprise a step of treating a subject with a therapeutically effective amount of an antibody, antibody fragment or variant thereof, comprising any one or plurality of CDR sequences identified as SEQ ID NO:553, or comprising CDR sequences that have at least about 70%, 75%, 80%, 85%, 90%, 95%, 96%, 97%, 98%, 99% or 100% sequence identity to CDR sequences identified as SEQ ID NO:553.

In some embodiments, the methods comprise a step of treating a subject with a therapeutically effective amount of an antibody, antibody fragment or variant thereof, comprising any one or plurality of CDR sequences identified as SEQ ID NO:554, or comprising CDR sequences that have at least about 70%, 75%, 80%, 85%, 90%, 95%, 96%, 97%, 98%, 99% or 100% sequence identity to CDR sequences identified as SEQ ID NO:554.

In some embodiments, the methods comprise a step of treating a subject with a therapeutically effective amount of an antibody, antibody fragment or variant thereof, comprising any one or plurality of CDR sequences identified as SEQ ID NO:555, or comprising CDR sequences that have at least about 70%, 75%, 80%, 85%, 90%, 95%, 96%, 97%, 98%, 99% or 100% sequence identity to CDR sequences identified as SEQ ID NO:555.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:561, or comprising CDR sequences that have at least about 70%, 75%, 80%, 85%, 90%, 95%, 96%, 97%, 98%, 99% or 100% sequence identity to CDR sequences identified as SEQ ID NO:561.

In some embodiments, the methods comprise a step of treating a subject with a therapeutically effective amount of an antibody, antibody fragment or variant thereof, comprising any one or plurality of CDR sequences identified as SEQ ID NO:562, or comprising CDR sequences that have at least about 70%, 75%, 80%, 85%, 90%, 95%, 96%, 97%, 98%, 99% or 100% sequence identity to CDR sequences identified as SEQ ID NO:562.

In some embodiments, the methods comprise a step of treating a subject with a therapeutically effective amount of an antibody, antibody fragment or variant thereof, comprising any one or plurality of CDR sequences identified as SEQ ID NO:563, or comprising CDR sequences that have at least about 70%, 75%, 80%, 85%, 90%, 95%, 96%, 97%, 98%, 99% or 100% sequence identity to CDR sequences identified as SEQ ID NO:563.

In some embodiments, the methods comprise a step of treating a subject with a therapeutically effective amount of an antibody, antibody fragment or variant thereof, comprising any one or plurality of CDR sequences identified as SEQ ID NO:564, or comprising CDR sequences that have at least about 70%, 75%, 80%, 85%, 90%, 95%, 96%, 97%, 98%, 99% or 100% sequence identity to CDR sequences identified as SEQ ID NO:564.

In some embodiments, the methods comprise a step of treating a subject with a therapeutically effective amount of an antibody, antibody fragment or variant thereof, comprising any one or plurality of CDR sequences identified as SEQ ID NO:565, or comprising CDR sequences that have at least about 70%, 75%, 80%, 85%, 90%, 95%, 96%, 97%, 98%, 99% or 100% sequence identity to CDR sequences identified as SEQ ID NO:565.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:567, or comprising CDR sequences that have at least about 70%, 75%, 80%, 85%, 90%, 95%, 96%, 97%, 98%, 99% or 100% sequence identity to CDR sequences identified as SEQ ID NO:567.

In some embodiments, the methods comprise a step of treating a subject with a therapeutically effective amount of an antibody, antibody fragment or variant thereof, comprising any one or plurality of CDR sequences identified as SEQ ID NO:568, or comprising CDR sequences that have at least about 70%, 75%, 80%, 85%, 90%, 95%, 96%, 97%, 98%, 99% or 100% sequence identity to CDR sequences identified as SEQ ID NO:568.

In some embodiments, the methods comprise a step of treating a subject with a therapeutically effective amount of an antibody, antibody fragment or variant thereof, comprising any one or plurality of CDR sequences identified as SEQ ID NO:573, or comprising CDR sequences that have at least about 70%, 75%, 80%, 85%, 90%, 95%, 96%, 97%, 98%, 99% or 100% sequence identity to CDR sequences identified as SEQ ID NO:573.

In some embodiments, the methods comprise a step of treating a subject with a therapeutically effective amount of an antibody, antibody fragment or variant thereof, comprising any one or plurality of CDR sequences identified as SEQ ID NO:574, or comprising CDR sequences that have at least about 70%, 75%, 80%, 85%, 90%, 95%, 96%, 97%, 98%, 99% or 100% sequence identity to CDR sequences identified as SEQ ID NO:574.

In some embodiments, the methods comprise a step of treating a subject with a therapeutically effective amount of an antibody, antibody fragment or variant thereof, comprising any one or plurality of CDR sequences identified as SEQ ID NO:575, or comprising CDR sequences that have at least about 70%, 75%, 80%, 85%, 90%, 95%, 96%, 97%, 98%, 99% or 100% sequence identity to CDR sequences identified as SEQ ID NO:575.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:577, or comprising CDR sequences that have at least about 70%, 75%, 80%, 85%, 90%, 95%, 96%, 97%, 98%, 99% or 100% sequence identity to CDR sequences identified as SEQ ID NO:577.

In some embodiments, the methods comprise a step of treating a subject with a therapeutically effective amount of an antibody, antibody fragment or variant thereof, comprising any one or plurality of CDR sequences identified as SEQ ID NO:578, or comprising CDR sequences that have at least about 70%, 75%, 80%, 85%, 90%, 95%, 96%, 97%, 98%, 99% or 100% sequence identity to CDR sequences identified as SEQ ID NO:578.

In some embodiments, the methods comprise a step of treating a subject with a therapeutically effective amount of an antibody, antibody fragment or variant thereof, comprising any one or plurality of CDR sequences identified as SEQ ID NO:579, or comprising CDR sequences that have at least about 70%, 75%, 80%, 85%, 90%, 95%, 96%, 97%, 98%, 99% or 100% sequence identity to CDR sequences identified as SEQ ID NO:579.

In some embodiments, the methods comprise a step of treating a subject with a therapeutically effective amount of an antibody, antibody fragment or variant thereof, comprising any one or plurality of CDR sequences identified as SEQ ID NO:580, or comprising CDR sequences that have at least about 70%, 75%, 80%, 85%, 90%, 95%, 96%, 97%, 98%, 99% or 100% sequence identity to CDR sequences identified as SEQ ID NO:580.

In some embodiments, the methods comprise a step of treating a subject with a therapeutically effective amount of an antibody, antibody fragment or variant thereof, comprising any one or plurality of CDR sequences identified as SEQ ID NO:581, or comprising CDR sequences that have at least about 70%, 75%, 80%, 85%, 90%, 95%, 96%, 97%, 98%, 99% or 100% sequence identity to CDR sequences identified as SEQ ID NO:581.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:587, or comprising CDR sequences that have at least about 70%, 75%, 80%, 85%, 90%, 95%, 96%, 97%, 98%, 99% or 100% sequence identity to CDR sequences identified as SEQ ID NO:587.

In some embodiments, the methods comprise a step of treating a subject with a therapeutically effective amount of an antibody, antibody fragment or variant thereof, comprising any one or plurality of CDR sequences identified as SEQ ID NO:588, or comprising CDR sequences that have at least about 70%, 75%, 80%, 85%, 90%, 95%, 96%, 97%, 98%, 99% or 100% sequence identity to CDR sequences identified as SEQ ID NO:588.

In some embodiments, the methods comprise a step of treating a subject with a therapeutically effective amount of an antibody, antibody fragment or variant thereof, comprising any one or plurality of CDR sequences identified as SEQ ID NO:589, or comprising CDR sequences that have at least about 70%, 75%, 80%, 85%, 90%, 95%, 96%, 97%, 98%, 99% or 100% sequence identity to CDR sequences identified as SEQ ID NO:589.

In some embodiments, the methods comprise a step of treating a subject with a therapeutically effective amount of an antibody, antibody fragment or variant thereof, comprising any one or plurality of CDR sequences identified as SEQ ID NO:590, or comprising CDR sequences that have at least about 70%, 75%, 80%, 85%, 90%, 95%, 96%, 97%, 98%, 99% or 100% sequence identity to CDR sequences identified as SEQ ID NO:590.

In some embodiments, the methods comprise a step of treating a subject with a therapeutically effective amount of an antibody, antibody fragment or variant thereof, comprising any one or plurality of CDR sequences identified as SEQ ID NO:594, or comprising CDR sequences that have at least about 70%, 75%, 80%, 85%, 90%, 95%, 96%, 97%, 98%, 99% or 100% sequence identity to CDR sequences identified as SEQ ID NO:594.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:600, or comprising CDR sequences that have at least about 70%, 75%, 80%, 85%, 90%, 95%, 96%, 97%, 98%, 99% or 100% sequence identity to CDR sequences identified as SEQ ID NO:600.

In some embodiments, the methods comprise a step of treating a subject with a therapeutically effective amount of an antibody, antibody fragment or variant thereof, comprising any one or plurality of CDR sequences identified as SEQ ID NO:601, or comprising CDR sequences that have at least about 70%, 75%, 80%, 85%, 90%, 95%, 96%, 97%, 98%, 99% or 100% sequence identity to CDR sequences identified as SEQ ID NO:601.

In some embodiments, the methods comprise a step of treating a subject with a therapeutically effective amount of an antibody, antibody fragment or variant thereof, comprising any one or plurality of CDR sequences identified as SEQ ID NO:602, or comprising CDR sequences that have at least about 70%, 75%, 80%, 85%, 90%, 95%, 96%, 97%, 98%, 99% or 100% sequence identity to CDR sequences identified as SEQ ID NO:602.

In some embodiments, the methods comprise a step of treating a subject with a therapeutically effective amount of an antibody, antibody fragment or variant thereof, comprising any one or plurality of CDR sequences identified as SEQ ID NO:603, or comprising CDR sequences that have at least about 70%, 75%, 80%, 85%, 90%, 95%, 96%, 97%, 98%, 99% or 100% sequence identity to CDR sequences identified as SEQ ID NO:603.

In some embodiments, the methods comprise a step of treating a subject with a therapeutically effective amount of an antibody, antibody fragment or variant thereof, comprising any one or plurality of CDR sequences identified as SEQ ID NO:604, or comprising CDR sequences that have at least about 70%, 75%, 80%, 85%, 90%, 95%, 96%, 97%, 98%, 99% or 100% sequence identity to CDR sequences identified as SEQ ID NO:604.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:606, or comprising CDR sequences that have at least about 70%, 75%, 80%, 85%, 90%, 95%, 96%, 97%, 98%, 99% or 100% sequence identity to CDR sequences identified as SEQ ID NO:606.

In some embodiments, the methods comprise a step of treating a subject with a therapeutically effective amount of an antibody, antibody fragment or variant thereof, comprising any one or plurality of CDR sequences identified as SEQ ID NO:607, or comprising CDR sequences that have at least about 70%, 75%, 80%, 85%, 90%, 95%, 96%, 97%, 98%, 99% or 100% sequence identity to CDR sequences identified as SEQ ID NO:607.

In some embodiments, the methods comprise a step of treating a subject with a therapeutically effective amount of an antibody, antibody fragment or variant thereof, comprising any one or plurality of CDR sequences identified as SEQ ID NO:612, or comprising CDR sequences that have at least about 70%, 75%, 80%, 85%, 90%, 95%, 96%, 97%, 98%, 99% or 100% sequence identity to CDR sequences identified as SEQ ID NO:612.

In some embodiments, the methods comprise a step of treating a subject with a therapeutically effective amount of an antibody, antibody fragment or variant thereof, comprising any one or plurality of CDR sequences identified as SEQ ID NO:613, or comprising CDR sequences that have at least about 70%, 75%, 80%, 85%, 90%, 95%, 96%, 97%, 98%, 99% or 100% sequence identity to CDR sequences identified as SEQ ID NO:613.

In some embodiments, the methods comprise a step of treating a subject with a therapeutically effective amount of an antibody, antibody fragment or variant thereof, comprising any one or plurality of CDR sequences identified as SEQ ID NO:614, or comprising CDR sequences that have at least about 70%, 75%, 80%, 85%, 90%, 95%, 96%, 97%, 98%, 99% or 100% sequence identity to CDR sequences identified as SEQ ID NO:614.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:616, or comprising CDR sequences that have at least about 70%, 75%, 80%, 85%, 90%, 95%, 96%, 97%, 98%, 99% or 100% sequence identity to CDR sequences identified as SEQ ID NO:616.

In some embodiments, the methods comprise a step of treating a subject with a therapeutically effective amount of an antibody, antibody fragment or variant thereof, comprising any one or plurality of CDR sequences identified as SEQ ID NO:617, or comprising CDR sequences that have at least about 70%, 75%, 80%, 85%, 90%, 95%, 96%, 97%, 98%, 99% or 100% sequence identity to CDR sequences identified as SEQ ID NO:617.

In some embodiments, the methods comprise a step of treating a subject with a therapeutically effective amount of an antibody, antibody fragment or variant thereof, comprising any one or plurality of CDR sequences identified as SEQ ID NO:618, or comprising CDR sequences that have at least about 70%, 75%, 80%, 85%, 90%, 95%, 96%, 97%, 98%, 99% or 100% sequence identity to CDR sequences identified as SEQ ID NO:618.

In some embodiments, the methods comprise a step of treating a subject with a therapeutically effective amount of an antibody, antibody fragment or variant thereof, comprising any one or plurality of CDR sequences identified as SEQ ID NO:619, or comprising CDR sequences that have at least about 70%, 75%, 80%, 85%, 90%, 95%, 96%, 97%, 98%, 99% or 100% sequence identity to CDR sequences identified as SEQ ID NO:619.

In some embodiments, the methods comprise a step of treating a subject with a therapeutically effective amount of an antibody, antibody fragment or variant thereof, comprising any one or plurality of CDR sequences identified as SEQ ID NO:620, or comprising CDR sequences that have at least about 70%, 75%, 80%, 85%, 90%, 95%, 96%, 97%, 98%, 99% or 100% sequence identity to CDR sequences identified as SEQ ID NO:620.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:626, or comprising CDR sequences that have at least about 70%, 75%, 80%, 85%, 90%, 95%, 96%, 97%, 98%, 99% or 100% sequence identity to CDR sequences identified as SEQ ID NO:626.

In some embodiments, the methods comprise a step of treating a subject with a therapeutically effective amount of an antibody, antibody fragment or variant thereof, comprising any one or plurality of CDR sequences identified as SEQ ID NO:627, or comprising CDR sequences that have at least about 70%, 75%, 80%, 85%, 90%, 95%, 96%, 97%, 98%, 99% or 100% sequence identity to CDR sequences identified as SEQ ID NO:627.

In some embodiments, the methods comprise a step of treating a subject with a therapeutically effective amount of an antibody, antibody fragment or variant thereof, comprising any one or plurality of CDR sequences identified as SEQ ID NO:628, or comprising CDR sequences that have at least about 70%, 75%, 80%, 85%, 90%, 95%, 96%, 97%, 98%, 99% or 100% sequence identity to CDR sequences identified as SEQ ID NO:628.

In some embodiments, the methods comprise a step of treating a subject with a therapeutically effective amount of an antibody, antibody fragment or variant thereof, comprising any one or plurality of CDR sequences identified as SEQ ID NO:629, or comprising CDR sequences that have at least about 70%, 75%, 80%, 85%, 90%, 95%, 96%, 97%, 98%, 99% or 100% sequence identity to CDR sequences identified as SEQ ID NO:629.

In some embodiments, the methods comprise a step of treating a subject with a therapeutically effective amount of an antibody, antibody fragment or variant thereof, comprising any one or plurality of CDR sequences identified as SEQ ID NO:630, or comprising CDR sequences that have at least about 70%, 75%, 80%, 85%, 90%, 95%, 96%, 97%, 98%, 99% or 100% sequence identity to CDR sequences identified as SEQ ID NO:630.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:632, or comprising CDR sequences that have at least about 70%, 75%, 80%, 85%, 90%, 95%, 96%, 97%, 98%, 99% or 100% sequence identity to CDR sequences identified as SEQ ID NO:632.

In some embodiments, the methods comprise a step of treating a subject with a therapeutically effective amount of an antibody, antibody fragment or variant thereof, comprising any one or plurality of CDR sequences identified as SEQ ID NO:633, or comprising CDR sequences that have at least about 70%, 75%, 80%, 85%, 90%, 95%, 96%, 97%, 98%, 99% or 100% sequence identity to CDR sequences identified as SEQ ID NO:633.

In some embodiments, the methods comprise a step of treating a subject with a therapeutically effective amount of an antibody, antibody fragment or variant thereof, comprising any one or plurality of CDR sequences identified as SEQ ID NO:638, or comprising CDR sequences that have at least about 70%, 75%, 80%, 85%, 90%, 95%, 96%, 97%, 98%, 99% or 100% sequence identity to CDR sequences identified as SEQ ID NO:638.

In some embodiments, the methods comprise a step of treating a subject with a therapeutically effective amount of an antibody, antibody fragment or variant thereof, comprising any one or plurality of CDR sequences identified as SEQ ID NO:639, or comprising CDR sequences that have at least about 70%, 75%, 80%, 85%, 90%, 95%, 96%, 97%, 98%, 99% or 100% sequence identity to CDR sequences identified as SEQ ID NO:639.

In some embodiments, the methods comprise a step of treating a subject with a therapeutically effective amount of an antibody, antibody fragment or variant thereof, comprising any one or plurality of CDR sequences identified as SEQ ID NO:640, or comprising CDR sequences that have at least about 70%, 75%, 80%, 85%, 90%, 95%, 96%, 97%, 98%, 99% or 100% sequence identity to CDR sequences identified as SEQ ID NO:640.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:642, or comprising CDR sequences that have at least about 70%, 75%, 80%, 85%, 90%, 95%, 96%, 97%, 98%, 99% or 100% sequence identity to CDR sequences identified as SEQ ID NO:642.

In some embodiments, the methods comprise a step of treating a subject with a therapeutically effective amount of an antibody, antibody fragment or variant thereof, comprising any one or plurality of CDR sequences identified as SEQ ID NO:643, or comprising CDR sequences that have at least about 70%, 75%, 80%, 85%, 90%, 95%, 96%, 97%, 98%, 99% or 100% sequence identity to CDR sequences identified as SEQ ID NO:643.

In some embodiments, the methods comprise a step of treating a subject with a therapeutically effective amount of an antibody, antibody fragment or variant thereof, comprising any one or plurality of CDR sequences identified as SEQ ID NO:644, or comprising CDR sequences that have at least about 70%, 75%, 80%, 85%, 90%, 95%, 96%, 97%, 98%, 99% or 100% sequence identity to CDR sequences identified as SEQ ID NO:644.

In some embodiments, the methods comprise a step of treating a subject with a therapeutically effective amount of an antibody, antibody fragment or variant thereof, comprising any one or plurality of CDR sequences identified as SEQ ID NO:645, or comprising CDR sequences that have at least about 70%, 75%, 80%, 85%, 90%, 95%, 96%, 97%, 98%, 99% or 100% sequence identity to CDR sequences identified as SEQ ID NO:645.

In some embodiments, the methods comprise a step of treating a subject with a therapeutically effective amount of an antibody, antibody fragment or variant thereof, comprising any one or plurality of CDR sequences identified as SEQ ID NO:646, or comprising CDR sequences that have at least about 70%, 75%, 80%, 85%, 90%, 95%, 96%, 97%, 98%, 99% or 100% sequence identity to CDR sequences identified as SEQ ID NO:646.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:652, or comprising CDR sequences that have at least about 70%, 75%, 80%, 85%, 90%, 95%, 96%, 97%, 98%, 99% or 100% sequence identity to CDR sequences identified as SEQ ID NO:652.

In some embodiments, the methods comprise a step of treating a subject with a therapeutically effective amount of an antibody, antibody fragment or variant thereof, comprising any one or plurality of CDR sequences identified as SEQ ID NO:653, or comprising CDR sequences that have at least about 70%, 75%, 80%, 85%, 90%, 95%, 96%, 97%, 98%, 99% or 100% sequence identity to CDR sequences identified as SEQ ID NO:653.

In some embodiments, the methods comprise a step of treating a subject with a therapeutically effective amount of an antibody, antibody fragment or variant thereof, comprising any one or plurality of CDR sequences identified as SEQ ID NO:654, or comprising CDR sequences that have at least about 70%, 75%, 80%, 85%, 90%, 95%, 96%, 97%, 98%, 99% or 100% sequence identity to CDR sequences identified as SEQ ID NO:654.

In some embodiments, the methods comprise a step of treating a subject with a therapeutically effective amount of an antibody, antibody fragment or variant thereof, comprising any one or plurality of CDR sequences identified as SEQ ID NO:655, or comprising CDR sequences that have at least about 70%, 75%, 80%, 85%, 90%, 95%, 96%, 97%, 98%, 99% or 100% sequence identity to CDR sequences identified as SEQ ID NO:655.

In some embodiments, the methods comprise a step of treating a subject with a therapeutically effective amount of an antibody, antibody fragment or variant thereof, comprising any one or plurality of CDR sequences identified as SEQ ID NO:656, or comprising CDR sequences that have at least about 70%, 75%, 80%, 85%, 90%, 95%, 96%, 97%, 98%, 99% or 100% sequence identity to CDR sequences identified as SEQ ID NO:656.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:658, or comprising CDR sequences that have at least about 70%, 75%, 80%, 85%, 90%, 95%, 96%, 97%, 98%, 99% or 100% sequence identity to CDR sequences identified as SEQ ID NO:658.

In some embodiments, the methods comprise a step of treating a subject with a therapeutically effective amount of an antibody, antibody fragment or variant thereof, comprising any one or plurality of CDR sequences identified as SEQ ID NO:659, or comprising CDR sequences that have at least about 70%, 75%, 80%, 85%, 90%, 95%, 96%, 97%, 98%, 99% or 100% sequence identity to CDR sequences identified as SEQ ID NO:659.

In some embodiments, the methods comprise a step of treating a subject with a therapeutically effective amount of an antibody, antibody fragment or variant thereof, comprising any one or plurality of CDR sequences identified as SEQ ID NO:664, or comprising CDR sequences that have at least about 70%, 75%, 80%, 85%, 90%, 95%, 96%, 97%, 98%, 99% or 100% sequence identity to CDR sequences identified as SEQ ID NO:664.

In some embodiments, the methods comprise a step of treating a subject with a therapeutically effective amount of an antibody, antibody fragment or variant thereof, comprising any one or plurality of CDR sequences identified as SEQ ID NO:665, or comprising CDR sequences that have at least about 70%, 75%, 80%, 85%, 90%, 95%, 96%, 97%, 98%, 99% or 100% sequence identity to CDR sequences identified as SEQ ID NO:665.

In some embodiments, the methods comprise a step of treating a subject with a therapeutically effective amount of an antibody, antibody fragment or variant thereof, comprising any one or plurality of CDR sequences identified as SEQ ID NO:666, or comprising CDR sequences that have at least about 70%, 75%, 80%, 85%, 90%, 95%, 96%, 97%, 98%, 99% or 100% sequence identity to CDR sequences identified as SEQ ID NO:666.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:668, or comprising CDR sequences that have at least about 70%, 75%, 80%, 85%, 90%, 95%, 96%, 97%, 98%, 99% or 100% sequence identity to CDR sequences identified as SEQ ID NO:668.

In some embodiments, the methods comprise a step of treating a subject with a therapeutically effective amount of an antibody, antibody fragment or variant thereof, comprising any one or plurality of CDR sequences identified as SEQ ID NO:669, or comprising CDR sequences that have at least about 70%, 75%, 80%, 85%, 90%, 95%, 96%, 97%, 98%, 99% or 100% sequence identity to CDR sequences identified as SEQ ID NO:669.

In some embodiments, the methods comprise a step of treating a subject with a therapeutically effective amount of an antibody, antibody fragment or variant thereof, comprising any one or plurality of CDR sequences identified as SEQ ID NO:670, or comprising CDR sequences that have at least about 70%, 75%, 80%, 85%, 90%, 95%, 96%, 97%, 98%, 99% or 100% sequence identity to CDR sequences identified as SEQ ID NO:670.

In some embodiments, the methods comprise a step of treating a subject with a therapeutically effective amount of an antibody, antibody fragment or variant thereof, comprising any one or plurality of CDR sequences identified as SEQ ID NO:671, or comprising CDR sequences that have at least about 70%, 75%, 80%, 85%, 90%, 95%, 96%, 97%, 98%, 99% or 100% sequence identity to CDR sequences identified as SEQ ID NO:671.

In some embodiments, the methods comprise a step of treating a subject with a therapeutically effective amount of an antibody, antibody fragment or variant thereof, comprising any one or plurality of CDR sequences identified as SEQ ID NO:672, or comprising CDR sequences that have at least about 70%, 75%, 80%, 85%, 90%, 95%, 96%, 97%, 98%, 99% or 100% sequence identity to CDR sequences identified as SEQ ID NO:672.

In some embodiments, the methods comprise a step of treating a subject with a therapeutically effective amount of an antibody, antibody fragment or variant thereof, comprising

In some embodiments, the methods comprise a step of treating a subject with a therapeutically effective amount of an antibody, antibody fragment or variant thereof, comprising any one or plurality of CDR sequences identified as SEQ ID NO:678, or comprising CDR sequences that have at least about 70%, 75%, 80%, 85%, 90%, 95%, 96%, 97%, 98%, 99% or 100% sequence identity to CDR sequences identified as SEQ ID NO:678.

In some embodiments, the methods comprise a step of treating a subject with a therapeutically effective amount of an antibody, antibody fragment or variant thereof, comprising any one or plurality of CDR sequences identified as SEQ ID NO:679, or comprising CDR sequences that have at least about 70%, 75%, 80%, 85%, 90%, 95%, 96%, 97%, 98%, 99% or 100% sequence identity to CDR sequences identified as SEQ ID NO:679.

In some embodiments, the methods comprise a step of treating a subject with a therapeutically effective amount of an antibody, antibody fragment or variant thereof, comprising any one or plurality of CDR sequences identified as SEQ ID NO:680, or comprising CDR sequences that have at least about 70%, 75%, 80%, 85%, 90%, 95%, 96%, 97%, 98%, 99% or 100% sequence identity to CDR sequences identified as SEQ ID NO:680.

In some embodiments, the methods comprise a step of treating a subject with a therapeutically effective amount of an antibody, antibody fragment or variant thereof, comprising any one or plurality of CDR sequences identified as SEQ ID NO:681, or comprising CDR sequences that have at least about 70%, 75%, 80%, 85%, 90%, 95%, 96%, 97%, 98%, 99% or 100% sequence identity to CDR sequences identified as SEQ ID NO:681.

In some embodiments, the methods comprise a step of treating a subject with a therapeutically effective amount of an antibody, antibody fragment or variant thereof, comprising any one or plurality of CDR sequences identified as SEQ ID NO:682, or comprising CDR sequences that have at least about 70%, 75%, 80%, 85%, 90%, 95%, 96%, 97%, 98%, 99% or 100% sequence identity to CDR sequences identified as SEQ ID NO:682.

In some embodiments, the methods comprise a step of treating a subject with a therapeutically effective amount of an antibody, antibody fragment or variant thereof, comprising any one or plurality of CDR sequences identified as SEQ ID NO:683, or comprising CDR sequences that have at least about 70%, 75%, 80%, 85%, 90%, 95%, 96%, 97%, 98%, 99% or 100% sequence identity to CDR sequences identified as SEQ ID NO:683.

In some embodiments, the anti-CD32b antigen-binding fragment, variant, or derivative thereof, is a Fc fragment or a Fv fragment. In some embodiments, the anti-CD32b antibodies, or antigen-binding fragment, variant, or derivative thereof, further comprises a heavy chain constant region. In some embodiments, the anti-CD32b antibodies, or antigen-binding fragment, variant, or derivative thereof, further comprises a light chain constant region.

In one particular embodiment, the antibody is derived from a mouse monoclonal antibody produced by hybridoma clone, having ATCC accession number PTA-7610. Hybridomas producing antibodies 8B5.3.4 have been deposited with the American Type Culture Collection (10801 University Blvd., Manassas, Va. 20110-2209) on May 23, 2006 under the provisions of the Budapest Treaty on the International Recognition of the Deposit of Microorganisms for the Purposes of Patent Procedures, and assigned accession number PTA-7610 (for hybridoma producing the 8B5.3.4 antibody), and are incorporated herein by reference. In a specific embodiment, the invention encompasses an antibody with the heavy chain variable region having the amino acid sequence of SEQ ID NO: 4 and the light chain variable region having the amino acid sequence of SEQ ID NO: 3. In a preferred embodiment, the antibodies of the invention are human or have been humanized, preferably a humanized version of the antibody produced by hybridoma clone 8B5.3.4.

The invention also encompasses the use of other antibodies, preferably monoclonal antibodies or fragments thereof that specifically bind FcγRIIB, preferably human FcγRIIB, more preferably native human FcγRIIB, that are derived from clones including but not limited to 2B6 and 3H7, 1D5, 2E1, 2H9, 2D11, and 1F2 having ATCC Accession numbers, PTA-4591, PTA-4592, PTA-5958, PTA-5961, PTA-5962, PTA-5960, and PTA-5959, respectively. Hybridomas producing the 2B6 and 3H7 clones were deposited under the provisions of the Budapest Treaty with the American Type Culture Collection (10801 University Blvd., Manassas, Va. 20110-2209) on Aug. 13, 2002, and are incorporated herein by reference. Hybridomas producing the 1D5, 2E1, 2H9, 2D11, and 1F2 clones were deposited under the provisions of the Budapest Treaty with the American Type Culture Collection (10801 University Blvd., Manassas, Va. 20110-2209) on May 7, 2004, and are incorporated herein by reference. In preferred embodiments, the antibodies described above are chimerized or humanized.

In a specific embodiment, an antibody used in the methods of the present invention is an antibody or an antigen-binding fragment thereof (e.g., comprising one or more complementarily determining regions (CDRs), preferably all 6 CDRs) of the antibody produced by clone 8B5.3.4 with ATCC accession number PTA-7610 (e.g., the heavy chain CDR3). In a specific embodiment, an antibody used in the methods of the present invention is an antibody or an antigen-binding fragment thereof (e.g., comprising one or more complementarily determining regions (CDRs), preferably all 6 CDRs) of the antibody produced by clone 2B6, 3H7, 1D5, 2E1, 2H9, 2D11, and 1F2 having ATCC Accession numbers, PTA-4591, PTA-4592, PTA-5958, PTA-5961, PTA-5962, PTA-5960, and PTA-5959, respectively (e.g., the heavy chain CDR3). Antibodies or antigen-binding fragments thereof comprising less than 6 CDRs with high affinity and specificity for a particular antigen as well as methods for producing and identifying such antibodies are commonly known in the art (see, e.g., Ward et al., 1989, Nature 341:544-546; Dumoulin et al., 2002, Protein Sci. 11:500-515; Davies et al., 1995, Bio/Technol. 13:475-479; Van den Beucken et al., 2001, J. Mol. Biol. 310:591-601; and Pereira et al., 1998, Biochem. 37:1430-1437, all of which are incorporated by reference herein in their entireties). Antibodies specific for a particular antigen that were generated by combining one or two CDRs from an antibody known to specifically bind to the antigen with other CDRs have been described and are commonly known in the art (see, e.g., Marks et al., 1992, Bio/Technol. 10:779-783; Klimka et al., 2000, Brit. J. Cancer 83 (2): 252-260; and Rader et al., 1998, Proc. Natl. Acad. Sci. USA 95:8910-8915, all of which are incorporated by reference herein in their entireties). Thus, the invention contemplates antibodies having one, two, three, four, or five of the CDRs of clone 8B5.3.4 that bind FcγRIIB specifically and which may be identified using the screening methods disclosed herein.

Techniques are known for deriving an antibody of a different subclass or isotype from an antibody of interest, i.e., subclass switching. Thus, IgG antibodies may be derived from an IgM antibody, for example, and vice versa. Such techniques allow the preparation of new antibodies that possess the antigen-binding properties of a given antibody (the parent antibody), but also exhibit biological properties associated with an antibody isotype or subclass different from that of the parent antibody. Recombinant DNA techniques may be employed. Cloned DNA encoding particular antibody polypeptides may be employed in such procedures, e.g., DNA encoding the constant domain of an antibody of the desired isotype (Lantto et al., 2002, Methods Mol. Biol. 178:303-16).

The monoclonal antibody, or the antigen-binding fragment thereof, of the disclosure can comprise one or more amino acid substitutions, insertions and/or deletions in the framework and/or CDR regions of the heavy and light chain variable domains. Such mutations can be readily ascertained by comparing the amino acid sequences disclosed herein to sequences available from, for example, public antibody sequence databases. Once obtained, antibodies and antigen-binding fragments that contain one or more mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present disclosure.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm, which has been incorporated into the GAP program in the GCG software package (available at gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present disclosure can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the molecules of the disclosure. To obtain gapped alignments for comparison purposes, Gapped BLAST can be utilized as described in Altschul et al, (1997) Nucleic Acids Res. 25 (17): 3389-3402. When utilizing BLAST and Gapped BLAST programs, the default parameters of the respective programs (e.g., XBLAST and NBLAST) can be used (see ncbi.nlm.nih.gov).

Other modifications of the antibody are contemplated herein. For example, the antibody can be linked to one of a variety of nonproteinaceous polymers, for example, polyethylene glycol, polypropylene glycol, polyoxyalkylenes, or copolymers of polyethylene glycol and polypropylene glycol. The antibody also can be entrapped in microcapsules prepared, for example, by coacervation techniques or by interfacial polymerization (for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules), or in macroemulsions. Such techniques are disclosed in, for example, Remington's Pharmaceutical Sciences, 16th edition, Oslo, A., Ed., (1980).

Variant antibodies and salts thereof also are included within the scope of the disclosure. Variants of the sequences recited in the application also are included within the scope of the disclosure. Further variants of the antibody sequences having improved affinity can be obtained using methods known in the art and are included within the scope of the disclosure. For example, amino acid substitutions can be used to obtain antibodies with further improved affinity. Alternatively, codon optimization of the nucleotide sequence can be used to improve the efficiency of translation in expression systems for the production of the antibody. Variants may include non-natural amino acids up to a certain percentage. In some embodiments, the antibody comprises a variant amino acid sequence comprising about 1, 2, 3, 4, 5, 6, 7, 8, 9, 10, 11, 12, 13, 14, 15, 16, 17, 18, 19, 20 or more percent of non-natural amino acids.

In some embodiments, the anti-CD32b antibodies can be fully human (non-naturally occurring) antibodies. Methods for generating monoclonal antibodies, including fully human monoclonal antibodies are known in the art. Any such known methods can be used in the context of the present disclosure to make human antibodies that specifically bind to CD32b.

In some embodiments, the anti-CD32b antibodies, or antigen-binding fragment, variant, or derivative thereof, useful for the methods of the disclosure selectively binds human CD32b over human CD32a.

In some embodiments, the anti-CD32b antibody, or antigen-binding fragment, variant, or derivative thereof, useful for the methods of the disclosure is an IgG selected from the group consisting of an IgG1, an IgG2, an IgG3 and an IgG4. In some embodiments, the anti-CD32b antibody, or antigen-binding fragment, variant, or derivative thereof, is selected from the group consisting of: a monoclonal antibody, a chimeric antibody, a single chain antibody, a Fab, a Fc, and a scFv. In some embodiments, the anti-CD32b antibody, or antigen-binding fragment, variant, or derivative thereof, is a monoclonal antibody. In some embodiments, the anti-CD32b antibody, or antigen-binding fragment, variant, or derivative thereof, is a chimeric antibody. In some embodiments, the anti-CD32b antibody, or antigen-binding fragment, variant, or derivative thereof, is a single chain antibody. In some embodiments, the anti-CD32b antibody, or antigen-binding fragment, variant, or derivative thereof, is a Fab. In some embodiments, the anti-CD32b antibody, or antigen-binding fragment, variant, or derivative thereof, is a Fc. In some embodiments, the anti-CD32b antibody, or antigen-binding fragment, variant, or derivative thereof, is a scFv. In some embodiments, the anti-CD32b antibody, or antigen-binding fragment, variant, or derivative thereof, are chimeric, humanized or fully human.

In some embodiments, the anti-CD32b antibody, or antigen-binding fragment, variant, or derivative thereof, useful for the methods of the disclosure inhibits binding of human CD32b to immunoglobulin Fc domains.

In some embodiments, the anti-CD32b antibody, or antigen-binding fragment, variant, or derivative thereof, useful for the methods of the disclosure is a component of an immunoconjugate.

### Methods

The disclosure relates to methods of treating or preventing severity of Coronavirdae infection comprising administering to a subject in need thereof a pharmaceutical composition comprising a therapeutically effective amount of an agonist or antagonist of CD32 and a pharmaceutically acceptable carrier.

The disclosure also relates to a method of reducing the severity of Coronoaviridae infection comprising administering to a subject in need thereof a pharmaceutical composition comprising a therapeutically effective amount of an agonist or antagonist of CD32 and a pharmaceutically acceptable carrier.

The disclosure relates to a method of treating acute respiratory disorder comprising administering to a subject in need thereof a pharmaceutical composition comprising a therapeutically effective amount of an agonist or antagonist of CD32 and a pharmaceutically acceptable carrier.

The disclosure relates to a method of treating immune-complex-mediated autoimmunity comprising administering to a subject in need thereof a pharmaceutical composition comprising a therapeutically effective amount of an agonist or antagonist of CD32 and a pharmaceutically acceptable carrier.

The disclosure relates to a method of treating immune-complex-mediated autoimmunity comprising administering to a subject in need thereof a pharmaceutical composition comprising a therapeutically effective amount of an agonist or antagonist of CD32 and a pharmaceutically acceptable carrier.

The disclosure relates to a method of treating immune-complex-mediated autoimmunity associated with viral infection, septic shock or acute respiratory inflammation comprising administering to a subject in need thereof a pharmaceutical composition comprising a therapeutically effective amount of an agonist or antagonist of CD32 and a pharmaceutically acceptable carrier.

In some embodiments, the above methods comprise administering to a subject in need thereof a pharmaceutical composition comprising a therapeutically effective amount of an antagonist of CD32b and a pharmaceutically acceptable carrier. In some embodiments, the antagonist of CD32b is an anti-CD32b antibody, or antigen-binding fragment, variant, or derivative thereof. In some embodiments, the antagonist for CD32b is an anti-CD32b antibody, or antigen-binding fragment, variant, or derivative thereof, that is selected for binding to CD32b but does not bind to CD32a.

### Pharmaceutical Compositions and Administration Methods

Methods of preparing and administering anti-CD32b antibodies, or antigen-binding fragments, variants, or derivatives thereof of the disclosure to a subject in need thereof are well known to or are readily determined by those skilled in the art. The route of administration of the anti-CD32b antibodies, or antigen-binding fragment, variant, or derivative thereof may be, for example, oral, parenteral, by inhalation or topical. The term parenteral as used herein includes, e.g., intravenous, intraarterial, intraperitoneal, intramuscular, subcutaneous, rectal or vaginal administration. While all these forms of administration are clearly contemplated as being within the scope of the disclosure, a form for administration would be a solution for injection, in particular for intravenous or intraarterial injection or drip. Usually, a suitable pharmaceutical composition for injection may comprise a buffer (e.g., acetate, phosphate or citrate buffer), a surfactant (e.g., polysorbate), optionally a stabilizer agent (e.g., human albumin), etc. However, in other methods compatible with the teachings herein, anti-CD32b antibodies, or antigen-binding fragments, variants, or derivatives thereof of the disclosure can be delivered directly to the site of the adverse cellular population thereby increasing the exposure of the diseased tissue to the therapeutic agent.

In some embodiments, anti-CD32b antibodies, or antigen-binding fragments, variants, or derivatives thereof of the disclosure are administered in an amount sufficient to deplete a population of B cells. B-cell population depletion can be measured and observed by methods that are known in the art without undue experimentation. In this regard, it will be appreciated that the disclosed antibodies will be formulated so as to facilitate administration and promote stability of the active agent.

In some embodiments, pharmaceutical compositions in accordance with the present disclosure comprise a pharmaceutically acceptable, non-toxic, sterile carrier such as physiological saline, non-toxic buffers, preservatives and the like. For the purposes of the instant application, “an effective amount” of an anti-CD32b antibody, or antigen-binding fragment, variant, or derivative thereof, conjugated or unconjugated, shall be held to mean an amount sufficient to achieve effective binding to a target and to achieve a desired goal benefit, e.g., to ameliorate symptoms or to abrogate severity of a disease or disorder in an animal model of disease or to detect a substance or a cell or a particular physiological parameter.

The pharmaceutical compositions used in this disclosure may comprise pharmaceutically acceptable carriers, including, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Preparations for parenteral administration includes sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject disclosure, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., an anti-CD32b antibody, or antigen-binding fragment, variant, or derivative thereof, by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof.

Parenteral formulations may be a single bolus dose, an infusion or a loading bolus dose followed with a maintenance dose. These compositions may be administered at specific fixed or variable intervals, e.g., once a day, or on an “as needed” basis.

The amount of an anti-CD32b antibody, or fragment, variant, or derivative thereof that may be combined with the carrier materials to produce a single dosage form will vary depending upon the host treated and the particular mode of administration. The composition may be administered as a single dose, multiple doses or over an established period of time in an infusion. Dosage regimens also may be adjusted to provide the optimum desired response (e.g., a therapeutic or prophylactic response in a non-human subject to mimic the response for a corresponding therapy if administered to a human subject).

In keeping with the scope of the present disclosure, anti-CD32b antibodies, or antigen-binding fragments, variants, or derivatives thereof of the disclosure may be administered to a subject (e.g., a non-human animal model of disease) in accordance with the aforementioned methods of administration in an amount sufficient to produce a desired effect. The anti-CD32b antibodies, or antigen-binding fragments, variants, or derivatives thereof of the disclosure can be administered to a subject in a conventional dosage form prepared by combining the antibody with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of anti-CD32b antibodies, or antigen-binding fragments, variants, or derivatives thereof of the disclosure may prove to be particularly effective or may be of particular interest for study in an animal model of disease.

Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the present invention are dictated by and directly dependent on (a) the unique characteristics of the active compound and the particular therapeutic effect to be achieved, and (b) the limitations inherent in the art of compounding such an active compound for the treatment of sensitivity in individuals.

The efficient dosages and the dosage regimens for the anti-CD32b antibodies, or antigen-binding fragments, variants, or derivatives thereof of the disclosure, depend on the disease or condition to be treated and may be determined by the persons skilled in the art. An exemplary, non-limiting range for a therapeutically effective amount of a compound of the present disclosure is about 0.1-100 mg/kg, such as about 0.1-50 mg/kg, for example about 0.1-20 mg/kg, such as about 0.1-10 mg/kg, for instance about 0.5, about such as 0.3, about 1, or about 3 mg/kg.

A physician or veterinarian having ordinary skill in the art may readily determine and prescribe the effective amount of the pharmaceutical composition required. For example, the physician or veterinarian could start doses of the anti-CD32b antibody, or antigen-binding fragments, variants, or derivatives thereof, employed in the pharmaceutical composition at levels lower than that required in order to achieve the desired therapeutic effect and gradually increase the dosage until the desired effect is achieved. In general, a suitable daily dose of a composition of the present disclosure will be that amount of the compound which is the lowest dose effective to produce a therapeutic effect. Such an effective dose will generally depend upon the factors described above. Administration may e.g. be intravenous, intramuscular, intraperitoneal, or subcutaneous, and for instance administered proximal to the site of the target. If desired, the effective daily dose of a pharmaceutical composition may be administered as two, three, four, five, six or more sub-doses administered separately at appropriate intervals throughout the day, optionally, in unit dosage forms.

In some embodiments, the anti-CD32b antibodies, or antigen-binding fragments, variants, or derivatives thereof of the disclosure, may be administered by infusion in a weekly dosage of from 10 to 500 mg/m2, such as of from 200 to 400 mg/m2. Such administration may be repeated, e.g., about 1 to 8 times, such as about 3 to 5 times. The administration may be performed by continuous infusion over a period of from about 2 to about 24 hours, such as of from about 2 to about 12 hours.

In some embodiments, the anti-CD32b antibodies, or antigen-binding fragments, variants, or derivatives thereof of the disclosure, may be administered by slow continuous infusion over a long period, such as more than about 24 hours, in order to reduce toxic side effects.

In some embodiments, the anti-CD32b antibodies, or antigen-binding fragments, variants, or derivatives thereof of the disclosure, may be administered in a weekly dosage of from about 250 mg to about 2000 mg, such as for example about 300 mg, about 500 mg, about 700 mg, about 1000 mg, about 1500 mg or about 2000 mg, for up to about 8 times, such as from about 4 to about 6 times. The administration may be performed by continuous infusion over a period of from about 2 to about 24 hours, such as of from about 2 to about 12 hours. Such regimen may be repeated one or more times as necessary, for example, after about 6 months or about 12 months. The dosage may be determined or adjusted by measuring the amount of compound of the present invention in the blood upon administration by for instance taking out a biological sample and using anti-idiotypic antibodies which target the antigen binding region of the anti-CD32b antibodies, or antigen-binding fragments, variants, or derivatives thereof of the disclosure.

In some embodiments, the anti-CD32b antibodies, or antigen-binding fragments, variants, or derivatives thereof of the disclosure, may be administered by maintenance therapy, such as, e.g., about once a week for a period of about 6 months or more.

In some embodiments, the anti-CD32b antibodies, or antigen-binding fragments, variants, or derivatives thereof of the disclosure, may be administered by a regimen including one infusion of an anti-CD32b antibody, or antigen-binding fragments, variants, or derivatives thereof of the disclosure, followed by an infusion of an anti-CD32b antibody, or antigen-binding fragments, variants, or derivatives thereof of the disclosure, conjugated to a radioisotope. The regimen may be repeated, e.g., about 7 to about 9 days later.

As non-limiting examples, treatment according to the present disclosure may be provided as a daily dosage of an anti-CD32b antibody, or antigen-binding fragments, variants, or derivatives thereof in an amount of about 0.1-100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20 after initiation of treatment, or any combination thereof, using single or divided doses of every 24, 12, 8, 6, 4, or 2 hours, or any combination thereof.

In some embodiments, the dosage of anti-CD32b antibodies, or antigen-binding fragments, variants, or derivatives thereof of the disclosure, being administered may be from about 0.1 mg to about 15,000 mg, In some embodiments, the dosage of anti-CD32b antibodies, or antigen-binding fragments, variants, or derivatives thereof of the disclosure, being administered may be from about 0.5 mg to about 12,000 mg, In some embodiments, the dosage of anti-CD32b antibodies, or antigen-binding fragments, variants, or derivatives thereof of the disclosure, being administered may be from about 1 mg to about 10,000 mg, In some embodiments, the dosage of anti-CD32b antibodies, or antigen-binding fragments, variants, or derivatives thereof of the disclosure, being administered may be from about 5 mg to about 5,000 mg, In some embodiments, the dosage of anti-CD32b antibodies, or antigen-binding fragments, variants, or derivatives thereof of the disclosure, being administered may be from about 5 mg to about 1,000 mg, In some embodiments, the dosage of anti-CD32b antibodies, or antigen-binding fragments, variants, or derivatives thereof of the disclosure, being administered may be from about 10 mg to about 500 mg,

Effective doses of the compositions of the present disclosure vary depending upon many different factors, including means of administration, target site, physiological state of the subject, other medications administered, and whether what is the goal of the study in which the composition is being administered (e.g., testing a combination therapy for its effects in an animal disease model as a predictor of its efficacy or toxicity in a human subject, or testing the effect of B-cell depletion in a model of a particular disease or disorder). For B-cell depletion, the dosage can range, e.g., from about 0.0001 to about 100 mg/kg, from about 0.01 to about 10 mg/kg (e.g., 0.02 mg/kg, 0.25 mg/kg, 0.5 mg/kg, 0.75 mg/kg, 1 mg/kg, 2 mg/kg, 5 mg/kg, 10 mg/kg, etc.), of the host body weight. For example, dosages can be about 1 mg/kg body weight or about 10 mg/kg body weight or within the range of about 1 mg/kg to about 10 mg/kg. In some embodiments, dosages are at least about 1 mg/kg. Doses intermediate in the above ranges are also intended to be within the scope of the disclosure. Subjects can be administered such doses daily, on alternative days, weekly or according to any other schedule determined by empirical analysis. Exemplary dosage schedules include about 1-10 mg/kg or about 15 mg/kg on consecutive days, about 30 mg/kg on alternate days, or about 10 mg/kg or about 60 mg/kg weekly. In some methods, two or more monoclonal antibodies with different binding specificities are administered simultaneously, in which case the dosage of each antibody administered falls within the ranges indicated. In some embodiments, the compositions of the present disclosure are administered in an amount of about 10 mg/kg every other week.

Anti-CD32b antibodies, or antigen-binding fragments, variants, or derivatives thereof of the disclosure can be administered on multiple occasions. Intervals between single dosages can be weekly, monthly or yearly. Intervals can also be irregular as indicated by measuring blood levels of target polypeptide or target molecule in the subject. In some embodiments, dosage is adjusted to achieve a plasma polypeptide concentration of about 1 μg/ml to about 1000 μg/ml, and in some embodiments, about 1 μg/ml to about 30 μg/ml, or about 25 μg/ml to about 300 μg/ml. Alternatively, anti-CD32b antibodies, or antigen-binding fragments, variants, or derivatives thereof of the disclosure can be administered as a sustained release formulation, in which case less frequent administration is required. Dosage and frequency vary depending on the half-life of the antibody in the subject. The half-life of an Anti-CD32b antibody can also be prolonged via fusion to a stable polypeptide or moiety, e.g., albumin or PEG. In some embodiments, anti-CD32b antibodies, or antigen-binding fragments, variants, or derivatives thereof of the disclosure can be administered in unconjugated form. In some embodiments, anti-CD32b antibodies, or antigen-binding fragments, variants, or derivatives thereof of the disclosure can be administered multiple times in conjugated form. In some embodiments, anti-CD32b antibodies, or antigen-binding fragments, variants, or derivatives thereof of the disclosure can be administered in unconjugated form, then in conjugated form, or vice versa.

The compositions of the present disclosure may be administered by any suitable method, e.g., parenterally, intraventricularly, orally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the anti-CD32b antibodies, or antigen-binding fragments, variants, or derivatives thereof are administered in such a way that they cross the blood-brain barrier. This crossing can result from the physico-chemical properties inherent in the anti-CD32b antibody molecule itself, from other components in a pharmaceutical formulation, or from the use of a mechanical device such as a needle, cannula or surgical instruments to breach the blood-brain barrier. Where the anti-CD32b antibody is a molecule that does not inherently cross the blood-brain barrier, e.g., a fusion to a moiety that facilitates the crossing, suitable routes of administration are, e.g., intrathecal or intracranial, e.g., directly into a chronic lesion of MS or EAE. Where the anti-CD32b antibody is a molecule that inherently crosses the blood-brain barrier, the route of administration may be by one or more of the various routes described below. In some embodiments, anti-CD32b antibodies are administered as a sustained release composition or device, such as a Medipad™ device.

The compositions may also comprise an anti-CD32b antibody dispersed in a biocompatible carrier material that functions as a suitable delivery or support system for the compounds. Suitable examples of sustained release carriers include semipermeable polymer matrices in the form of shaped articles such as suppositories or capsules. Implantable or microcapsular sustained release matrices include polylactides, copolymers of L-glutamic acid and gamma-ethyl-L-glutamate; poly(2-hydroxyethyl-methacrylate), ethylene vinyl acetate, or poly-D-(−)-3hydroxybutyric acid.

Anti-CD32b antibodies, or antigen-binding fragments, variants, or derivatives thereof of the disclosure can optionally be administered in combination with other agents e.g., to be tested for toxicity or for efficacy, e.g., in treating or having an effect on the disorder or condition in an animal model of disease. The agents can be administered simultaneously or in any order, or with a time interval in between.

Although the disclosure has been described with reference to exemplary embodiments, it is not limited thereto. Those skilled in the art will appreciate that numerous changes and modifications may be made to the preferred embodiments of the disclosure and that such changes and modifications may be made without departing from the true spirit of the disclosure. It is therefore intended that the appended claims be construed to cover all such equivalent variations as fall within the true spirit and scope of the disclosure.

All referenced journal articles, patents, and other publications are incorporated by reference herein in their entireties. U.S. Publication Nos. 2008/0044429A1 and 2020/0362036A1 are hereby incorporated by reference in their entireties. PCT Publication Nos. WO 2015/173384A1 and WO 2017/103895A1 are hereby incorporated by reference in their entireties. U.S. Pat. Nos. 8,652,466 and 8,968,730 are hereby incorporated by reference in their entireties.

### EXAMPLES

### Example 1. Global Absence and Targeting of Protective Immune States in Severe COVID-19

While SARS-COV-2 infection has pleiotropic and systemic effects in some patients, many others experience milder symptoms. We sought a holistic understanding of the severe/mild distinction in COVID-19 pathology, and its origins. We performed a whole-blood preserving single-cell analysis protocol to integrate contributions from all major cell types including neutrophils, monocytes, platelets, lymphocytes and the contents of serum. Patients with mild COVID-19 disease display a coordinated pattern of interferon-stimulated gene (ISG) expression across every cell population and these cells are systemically absent in patients with severe disease. Severe COVID-19 patients also paradoxically produce very high anti-SARS-COV-2 antibody titers and have lower viral load as compared to mild disease. Examination of the serum from severe patients demonstrates that they uniquely produce antibodies that functionally block the production of the mild disease-associated ISG-expressing cells, by engaging conserved signaling circuits that dampen cellular responses to interferons. Overzealous antibody responses pit the immune system against itself in many COVID-19 patients and perhaps in other viral infections and this study defines targets for immunotherapies in severe patients to re-engage viral defense.

### 1. Materials and Methods

### I. Patients, Participants, Severity Score, and Clinical Data Collection

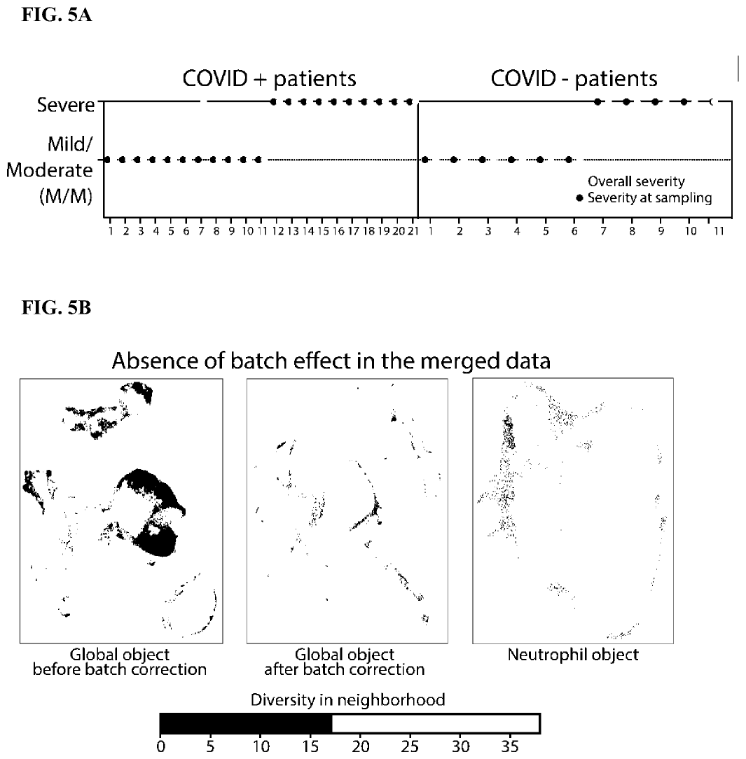

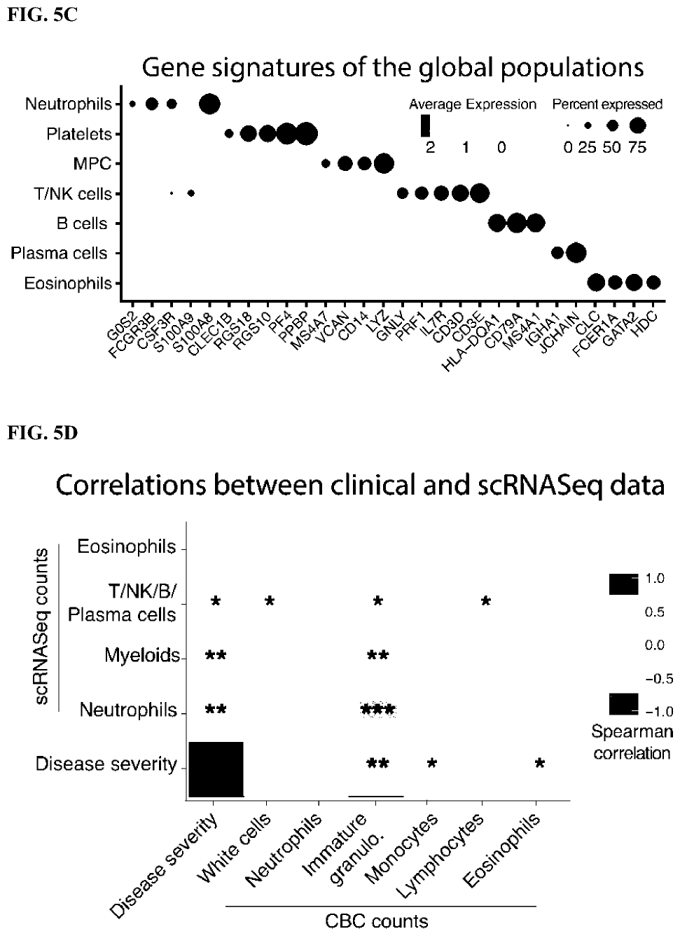

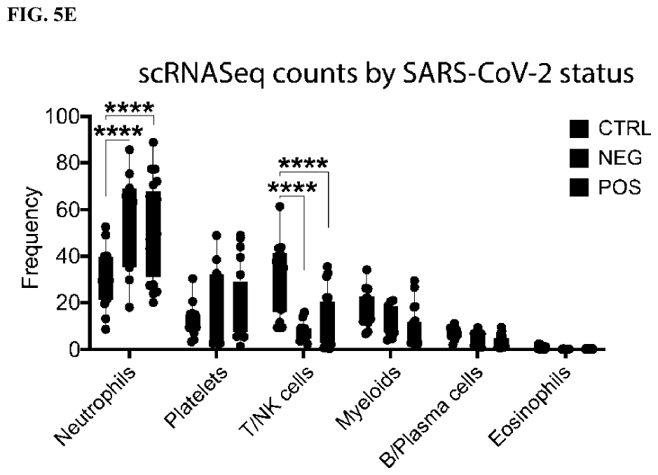

Patients admitted to the Hospital of the University of California with known or presumptive COVID-19 were screened within 3 days of hospitalization. Patients, or a designated surrogate, provided informed consent to participate in the study. This study includes a subset of patient enrolled between April 8 and May 1 in the COMET (COVID-19 Multi-immunophenotyping projects for Effective Therapies; comet-study.org/) study at UCSF. COMET is a prospective study that aims to describe the relationship between specific immunologic assessments and the clinical courses of COVID-19 in hospitalized patients. Healthy donors (Ctrl) were adults with no prior diagnosis of or recent symptoms consistent with COVID-19. This analysis includes samples from participants who provided informed consent directly, via a surrogate, or otherwise in accordance with protocols approved by the regional ethical research boards and the Declaration of Helsinki. For inpatients, clinical data were abstracted from the electronic medical record into standardized case report forms. We used both a severity score at the time of sampling and at the end of hospitalization (FIG. 5A). In both cases, severity assessment was based on three main parameters: level of care, need for mechanical ventilation, and time under mechanical ventilation. Mild/moderate patients are floor/ICU patients who did not require mechanical ventilation during their time of hospitalization and spent no more than 1 day in ICU. Severe patients are patients who required intensive care and mechanical ventilation (typically 5 days or more). Therefore, our validation cohort is composed of 21 COVID-19 positive patients (11 mild/moderate and 10 severe), 11 COVID-19 negative patient (6 mild/moderate and 5 severe), and 14 healthy participants. We also collected and used serum from a validation cohort composed of 14 SARS-CoV-2 positive. Samples were collected and severity was assessed as previously described for initial cohort. This discovery cohort is composed of 8 mild/moderate and 6 severe patients. Information on age, sex, type of infection, days of on onset, viral load, and CBC count are listed in Table 2. Patients were enrolled as described in Material and Methods section above and blood was collected from 4:30 AM rounds on the first day after admission. Healthy controls were from volunteers who fasted overnight and were collected between 6 am and 9 am. Data on individuals is shown along with average and standard deviations. The study is approved by the Institutional Review board: IRB #20-30497.

TABLE 2
COVID-19 Whole Blood Discovery Study Cohort.

| | | | | | Days between | | | | |
|---|---|---|---|---|---|---|---|---|---|
| | | | | | symptoms and | | ICU | | |
| | SARS- | | | Gender | sampling | | during | Days under | |
| | CoV-2 | Disease | | (1 = MALE, | (* = | Other | hospital | mechanical | |
| COMET ID | Status | Severity | Age | 2 = F) | asymptomatic) | Infection(s) | stay | Ventilation | Discharged |
| 1008 | NEG | Mild/ | 35.7 | 1 | 20 | Bacterial | No | 0 | Yes |
| | | Moderate | | | | | | | |
| 1016 | NEG | Mild/ | 63.5 | 2 | 4 | None | No | 0 | Yes |
| | | Moderate | | | | | | | |
| 1017 | NEG | Mild/ | 65.2 | 2 | 9 | None | No | 0 | Yes |
| | | Moderate | | | | | | | |
| 1045 | NEG | Mild/ | 82.6 | 1 | NA | None | Yes | 0 | Yes |
| | | Moderate | | | | | | | |
| 1049 | NEG | Mild/ | 68.8 | 2 | 1 | Bacterial | No | 0 | Yes |
| | | Moderate | | | | | | | |
| 1062 | NEG | Mild/ | 75.8 | 2 | NA | Bacterial | No | 0 | Yes |
| | | Moderate | | | | | | | |
| COVID | Average | | 65 | 1.7 | 9 | | | | |
| Negative Mild | Data: | | 16 | | 8 | | | | |
| n = 6 | SD: | | | | | | | | |
| 1009 | NEG | Severe | 67 | 1 | NA | Bacterial | Yes | 5 | yes |
| 1010 | NEG | Severe | 50.7 | 2 | 9 | Bacterial | Yes | 5 | Yes |
| 1020 | NEG | Severe | 78.6 | 1 | 5 | Bacterial | Yes | 2 | Yes |
| 1021 | NEG | Severe | 77 | 2 | 2 | Bacterial | Yes | 5 | Yes |
| 1046 | NEG | Severe | 49.6 | 1 | 3 | Bacterial | Yes | 1 | Yes |
| COVID | Average | | 65 | 1.4 | 5 | | | | |
| Negative | Data: | | | | | | | | |
| Severe n = 5 | SD: | | 14 | | 3 | | | | |
| 1005 | POS | Mild/ | 83.4 | 1 | 9 | None | No | 0 | Yes |
| | | Moderate | | | | | | | |
| 1006 | POS | Mild/ | 71.9 | 2 | 16 | None | No | 0 | Yes |
| | | Moderate | | | | | | | |
| 1007 | POS | Mild/ | 55.3 | 1 | 13 | Bacterial | No | 0 | Yes |
| | | Moderate | | | | | | | |
| 1012 | POS | Mild/ | 41.1 | 1 | 3 | None | No | 0 | Yes |
| | | Moderate | | | | | | | |
| 1014 | POS | Mild/ | 58.6 | 1 | 4 | None | Yes | 0 | Yes |
| | | Moderate | | | | | | | |
| 1025 | POS | Mild/ | 73.2 | 1 | NA* | None | No | 0 | Yes |
| | | Moderate | | | | | | | |
| 1026 | POS | Mild/ | 45.5 | 2 | 6 | None | No | 0 | Yes |
| | | Moderate | | | | | | | |
| 1029 | POS | Mild/ | 85 | 2 | 2 | Viral | No | 0 | Yes |
| | | Moderate | | | | | | | |
| 1044 | POS | Mild/ | 73.9 | 1 | NA* | None | No | 0 | Yes |
| | | Moderate | | | | | | | |
| 1052 | POS | Mild/ | 37.9 | 2 | 4 | Viral | No | 0 | Yes |
| | | Moderate | | | | | | | |
| 1054 | POS | Mild/ | 25.8 | 1 | NA* | None | No | 0 | Yes |
| | | Moderate | | | | | | | |
| COVID | Average | | 59 | 1.4 | 7 | | | | |
| Positive | Data: | | | | | | | | |
| Mild n = 11 | SD: | | 20 | | 5 | | | | |
| 1001 | POS | Severe | 34.8 | 2 | 12 | Viral | Yes | 10 | Yes |
| 1002 | POS | Severe | 79.6 | 1 | 4 | Bacterial | Yes | 7 | Yes |
| 1003 | POS | Severe | 43.6 | 2 | 13 | Viral | Yes | 5 | Yes |
| 1031 | POS | Severe | 44.2 | 1 | 20 | Viral | Yes | 28 | Yes |
| 1038 | POS | Severe | 61.4 | 2 | 14 | None | Yes | ND | Yes |
| 1047 | POS | Severe | 48.3 | 2 | 6 | Viral + | Yes | 28 | Yes |
| | | | | | | Bacterial | | | |
| 1050 | POS | Severe | 55 | 1 | 13 | Bacterial | Yes | 29 | Yes |
| 1051 | POS | Severe | 63.3 | 1 | 19 | Bacterial | Yes | 33 | Yes |
| 1055 | POS | Severe | 62.4 | 1 | 4 | Viral | Yes | 31 | Yes |
| 1060 | POS | Severe | 44.1 | 1 | 6 | Bacterial | Yes | 5 | Yes |
| COVID | Average | | 54 | 1.4 | 11 | | | | |
| Positive Severe | Data: | | 13 | | 6 | | | | |
| n = 10 | SD: | | | | | | | | |
| ICC_0001 | CTRL | CTRL | 39 | 1 | NA | NA | NA | NA | NA |
| ICC_0003 | CTRL | CTRL | 24 | 1 | NA | NA | NA | NA | NA |
| ICC_0666 | CTRL | CTRL | 45 | 2 | NA | NA | NA | NA | NA |
| ICC_1084 | CTRL | CTRL | 34 | 1 | NA | NA | NA | NA | NA |
| ICC_1117 | CTRL | CTRL | 41 | 1 | NA | NA | NA | NA | NA |
| ICC_1367 | CTRL | CTRL | 52 | 1 | NA | NA | NA | NA | NA |
| ICC_3231 | CTRL | CTRL | 28 | 2 | NA | NA | NA | NA | NA |
| ICC_3954 | CTRL | CTRL | 37 | 2 | NA | NA | NA | NA | NA |
| ICC_4096 | CTRL | CTRL | 31 | 1 | NA | NA | NA | NA | NA |
| ICC_4117 | CTRL | CTRL | 59 | 2 | NA | NA | NA | NA | NA |
| ICC_4319 | CTRL | CTRL | 30 | 1 | NA | NA | NA | NA | NA |
| ICC_4444 | CTRL | CTRL | 34 | 2 | NA | NA | NA | NA | NA |
| ICC_4923 | CTRL | CTRL | 34 | 1 | NA | NA | NA | NA | NA |
| Healthy | Average | | 38 | 1.4 | NA | | | | |
| Controls | Data: | | | | | | | | |
| n = 14 | SD: | | 9 | | NA | | | | |

ii. Isolation of Blood Cells and Processing for scRNA-Seq

ScRNA-seq was performed on fresh whole blood in order to preserve granulocytes. Briefly, peripheral blood was collected into EDTA tubes (BD, catalog no. 366643). Whole blood was prepared by treatment of 500 μL of peripheral blood with RBC lysis buffer (Roche, 11-814-389-001) according to manufacturer's procedures. Cells were then counted and 15.000 cells per individual were directly loaded in the Chromium™ Controller for partitioning single cells into nanoliter-scale Gel Bead-In-Emulsions (GEMs) following manufacturer's procedures (10× genomics). Some samples were pooled together (at 15,000 cells/sample) prior to GEM partitioning. Single Cell 5′ reagent kit v5.1 was used for reverse transcription, cDNA amplification and library construction of the gene expression libraries (10× Genomics) following the detailed protocol provided by 10× Genomics. Libraries were sequenced on an Illumina NovaSeq6000 using 28 cycles for R1 and 98 cycles for R2. All samples were encapsulated, and cDNA was generated within 6 hours after blood draw.

Iii. Bulk RNASeq Library Preparation for Genotyping:

RNA was extracted from aliquots of 250K Peripheral Blood Mononuclear Cells (PBMCs) utilizing the ZYMO Research Quick RNA MagBead kit (R2133) on a Thermofisher KingFisher Flex system following manufacturer's procedures. RNA integrity was inspected with Agilent Fragment Analyzer. Ribosomal and hemoglobin depleted total RNA-sequencing library were created using FastSelect (Qiagen cat #: 335377) and Tecan Universal Plus mRNA-Seq (0520-A01) with adaptations for automation of a Beckmen BioMek FXp system. Libraries were subsequently normalized and pooled for Illumina sequencing using a Labcyte Echo 525 system available at the Center for Advanced Technology at UCSF. The pooled libraries were sequenced on an Illumina NovaSeq S4 flow cell lane with paired end 150 bp reads.

iv. Computational Processing for Genotyping

Sequencing reads were aligned to the human reference genome and Ensembl annotation (GRCh38 genome build, Ensembl annotation version 95) using STAR v2.7.5c (PMID: 23104886) with the following parameters: —outFilterType BySJout—outFilterMismatchNoverLmax 0.04—outFilterMismatchNmax 999—alignSJDBoverhangMin 1—outFilterMultimapNmax 1—alignIntronMin 20—alignIntronMax 1000000—alignMatesGapMax 1000000. Duplicate reads were removed and read groups assigned by individual for variant calling using Picard Tools v2.23.3 (broadinstitute.github.io/picard/). Nucleotide variants were identified from the resulting bam files using the Genome Analysis Tool Kit (GATK, v4.0.11.0) following the best practices for RNA-seq variant calling (PMID: 25431634; PMID: 21478889). This include splitting spliced reads, calling variants with HaplotypeCaller (added parameters: —dont-use-soft-clipped-bases-stand-call-conf 20.0), and filtering variants with VariantFiltration (added parameters: -window 35-cluster 3—filter-name FS-filter FS>30.0—filter-name QD-filter QD<2.0). Variants were further filtered to include a list of high quality SNP for identification of the subject of origin of individual cells by removing all novel variants, maintaining only biallelic variants with MAF greater than 5%, a mix missing of one individual with a missing variant call at a specific site and requiring a minimum depth of two (parameters: —max-missing 1.0—min-alleles 2—max-alleles 2—remove-indels—snps snp.list.txt—min-meanDP 2—maf 0.05—recode—recode-INFO-all-out).

v. Data Pre-Processing of 10× Genomics Chromium scRNA-Seq Data

Sequencer-obtained bcl files were demultiplexed into individual sample the mkfastqs command on the Cellranger 3.0.2 suite of tools (https://support.10×genomics.com). Feature-barcode matrices were obtained for each sample by aligning the raw fastqs to GRCh38 reference genome (annotated with Ensembl v85) using the Cellranger count. Raw feature-barcode matrices were loaded into Seurat 3.1.5 (27) and genes with fewer than 3 UMIs were dropped from the analyses. Matrices were further filtered to remove events with greater than 20% percent mitochondrial content, events with greater than 50% ribosomal content, or events with fewer than 100 total genes. The cell cycle state of each cell was assessed using a published set of genes associated with various stages of human mitosis (28).

vi. Inter-Sample Doublet Detection

Libraries containing samples pooled prior to loading were processed using Freemuxlet (github.com/statgen/popscle), the genotype-free version of Demuxlet (29), to identify clusters of cells belonging to the same patient via SNP concordance. Briefly, the aligned reads from Cellranger were filtered to retain reads overlapping a high-quality list of SNPs obtained from the 1000 Genomes Consortium (1 KG) (30). Freemuxlet was run on this filtered bam using the 1 KG vcf file as a reference, the input number of samples/pool as a guideline for clustering groups of cells by SNP concordance, and all other default parameters. Cells are classified as singlets arising from a single library, doublets arising from two or more libraries, or as ambiguous cells that cannot be accurately assigned to any existing cluster (due to a lack of sufficient genetic information). Clusters of cells belonging to a unique sample were mapped to patients using their individual Freemuxlet-generated genotype, and ground truth genotypes per patient identified via bulk RNASeq. The pairwise discordance between inferred and ground-truth genotypes was assessed using the bcftools gtcheck command (31). Ambiguous, and doublet events were filtered from the major analysis (see platelet-first analysis).

vii. Data Quality Control and Normalization

The filtered count matrices were normalized, and variance stabilized using negative binomial regression via the scTransform method offered by Seurat (32). The effects of mitochondrial content, ribosomal content, and cell cycle state were regressed out of the normalized data to prevent any confounding signal. The normalized matrices were reduced to a lower dimension using Principal Component Analyses (PCA) and the first 30 principal coordinates per sample were subjected to a non-linear dimensionality reduction using Uniform Manifold Approximation and Projection (UMAP). Clusters of cells sharing similar transcriptomic signal were identified using the Louvain algorithm, and clustering resolutions varied between 0.6 and 1.2 based on the number and variety of cells obtained in the datasets. Clusters were loosely grouped into major cell types (T/NK, B/Plasma, mononuclear phagocytes, Neutrophil, Platelet, and Erythrocytes) using a curated list of 5 genes per cell type (5-gene signature) and the Seurat AddModuleScore function. Briefly, genes in the library are binned into one of 12 bins based on average expression in the dataset. The average expression of the genes in each signature are compared to a background list of randomly selected from the bins and used to generate a score per cell for each signature.

viii. Intra-Sample Heterotypic Doublet Detection

All libraries were further processed to identify heterotypic doublets arising from the 10× sample loading. Processed, annotated Seurat objects were processed using the DoubletFinder package (33). Briefly, the cells from the object are modified to generate artificial duplicates, and true doublets in the dataset are identified based on similarity to the artificial doublets in the modified gene space. The prior doublet rate per library was approximated using the information provided in the 10× knowledgebase (kb.10× genomics.com/hc/en-us/articles/360001378811) and this was corrected to account for homotypic doublets using the per-cluster numbers in each dataset. Heterotypic doublets were removed from the major analysis (see platelet-first analysis).

Ix. Data Integration and Batch Correction

The individual processed objects per library were filtered to remove Erythrocyte contamination. The raw and log-normalized counts per library were then pruned to retain only genes shared by all libraries. Pruned counts matrices were merged into a single Seurat object and the batch (or library) of origin was stored in the metadata of the object. The log-normalized counts were reduced to a lower dimension using PCA and the individual libraries were aligned in the shared PCA space in a batch-aware manner (each individual library was considered a batch) using the Harmony algorithm (34). The resulting Harmony components were used to generate a batch corrected UMAP, and to identify clusters of transcriptionally similar cells. Clusters were broadly labeled based on the 5-gene signature (FIG. 5) using a modified, bootstrapped version of the Seurat AddModuleScore to account for the numerous sequencing batches in our dataset. The modified function ran the AddModuleScore on random subsets of the data (subsampling rate=0.6) 10 times and averaged the score to provide a stable score per signature. A Seurat object was generated for each broad cell type containing clusters scoring highly for that cell type. Each broad cell-type object was subjected to the same harmony analysis to generate batch-aware log normalized counts that were used for visualization and subtype identification. To visualize the effect of harmonizing our single cell data, we identified the library diversity in the neighborhood of every cell on the plot. The neighborhood of a cell is defined as the collection of n nearest neighbors in the UMAP space (where n=sqrt (total cells)), pruned to retain cells lying within the 90th percentile of all calculated neighbor distances. The diversity is the set of all libraries represented within the neighborhood.

x. Differential Expression Tests and Cluster Marker Genes, Cluster Annotation and Volcano Plot

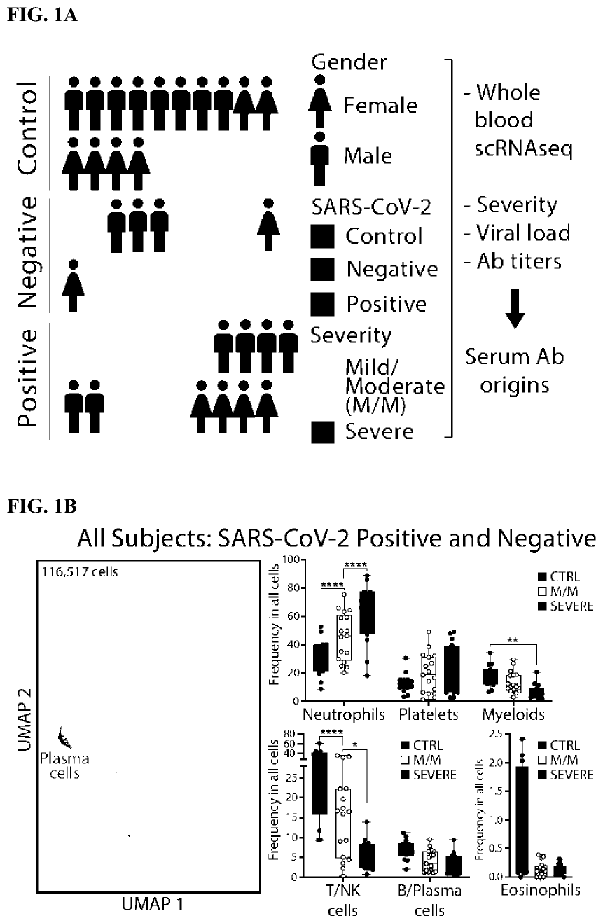

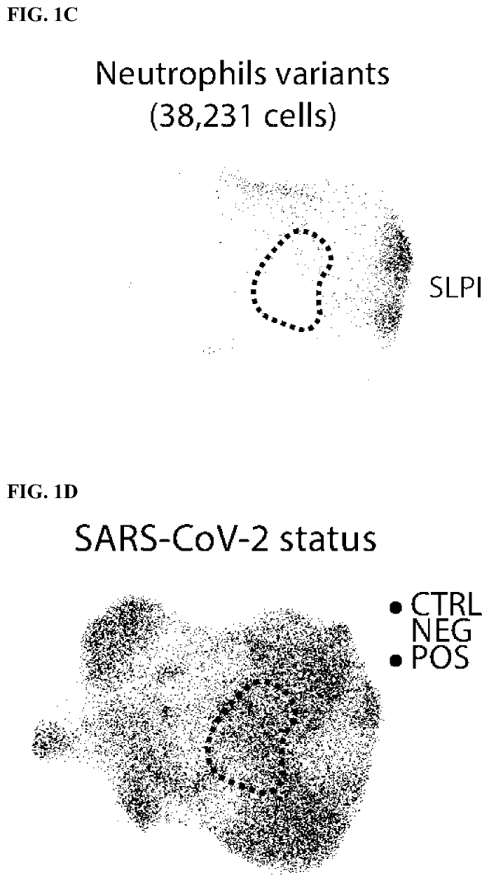

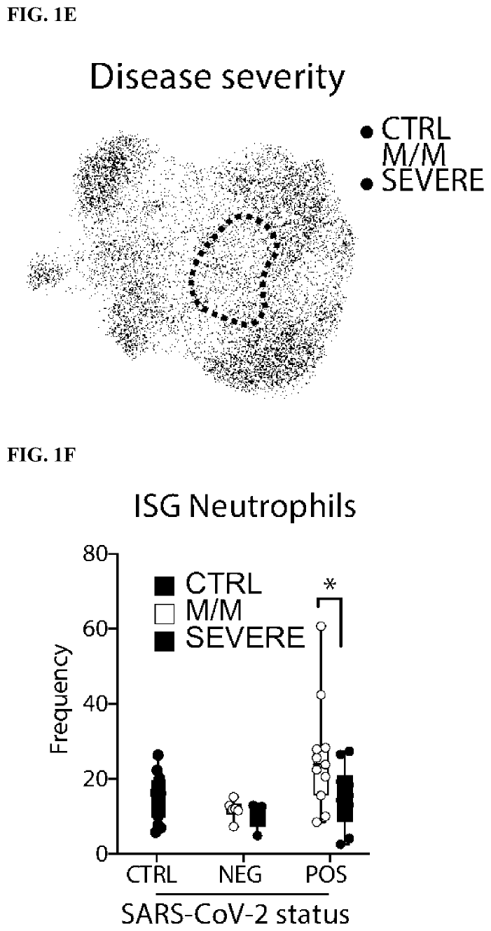

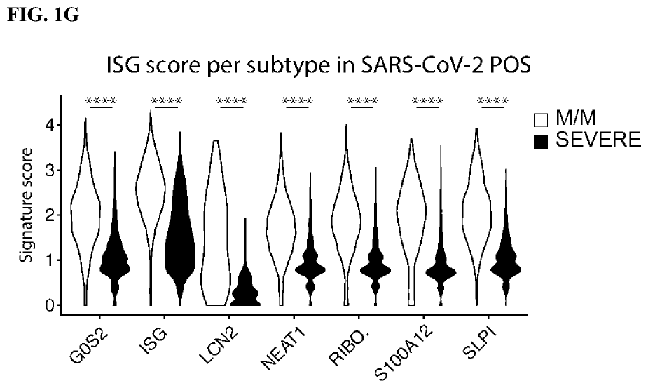

Differential gene expression (DGE) tests were performed on log-normalized gene counts using the Poisson test (with a latent batch variable to account for multiple library preparations) provided by the FindMarkers/FindAllMarkers functions in Seurat. Genes with >0.35 log-fold changes, an adjusted p value of 0.05 (based on Bonferroni correction), at least 25% expressed in tested groups, were regarded as significantly differentially expressed genes (DEGs). Cluster marker genes were identified by applying the DE tests for upregulated genes between cells in one cluster to all other clusters in the dataset. Top ranked genes (by log-fold changes) from each cluster of interest were extracted for further illustration. The exact number and definition of samples used in the analysis are specified in the legend of FIG. 1-3 and summarized in Table 2. The neutrophils, mononuclear phagocytic cells, T cells, B cells and platelets subtypes were identified by comparing cluster marker genes with public sources referenced in the text. The R package EnrichR were used to generate volcano plot from differential gene expression using FindMarkers function in Seurat.

xi. Platelet First scSeq Analysis

To identify the differential coagulation of platelets, we reintroduced heterotypic doublets to each library and filtered them to extract cells expressing at least 1 UMI of PF4 or PPBP, both platelet-specific marker genes. The raw and log-normalized counts per library were integrated using Harmony and processed as above. Broad cell types were identified using the score generated with the bootstrapped AddModuleScore and the per-sample rate of platelet aggregation with each cell types was inferred to be the fractions of cell counts in this dataset to the fractions of cell counts in the overall analysis. Significance testing was conducted using a non-parametric Kruskal-Wallis test with multiple comparisons.

xii. Monocle Analysis

Raw counts from the Individual cell-specific were used to create a monocle3_35-37) cell_data_set object, and the batch-corrected PCA and UMAP embeddings were imported directly from the Seurat object. Each cell-specific trajectory was inferred by reverse embedding the UMAP coordinates using the DDRTree method. The root cell states for the trajectory in monocytes and neutrophils were chosen based on literature, and for platelet cell based on the signature list defined in FIG. 5. Relative pseudotime was obtained through a linear transformation relative to the cells with the lowest and highest pseudotimes ((1-min pseudotime) max pseudotime).

xiii. Generation of Gene Expression Scores

ISG and Degranulation scores were generated by taking the mean of log-normalized expression for a particular set of genes related to the specific pathway or phenotype. The following gene lists were used to generate the scores-ISG: MT2A, ISG15, LY6E, IFITI, IFIT2, IFIT3, IFITM1, IFITM3, IF144L, IF16, MX1, IFI27; Degranulation: 486 genes from Neutrophils degranulation GO term #GO: 0043312. To visualize the distribution of these scores across cells, we binarized the distribution of the score at the 75th percentile and overlaid on the calculated UMAP coordinates.

xiv. Correlation Plots and Heatmap Visualization

Correlation coefficients used in variable against variable comparisons were calculated using Spearman's method to avoid assumption of linearity. Significance testing of correlation was performed with the following tests: Spearman's for continuous v. continuous, Kruskal-Wallis or Wilcoxon rank sum test for categorical v. continuous depending on number of categories and Fisher's exact for categorical v. categorical comparisons. Unless otherwise specified, variables on both axes were hierarchically clustered based on the distance matrix computed from the correlation coefficient.

xv. Embedding a Low-Dimensional Representation of Patients Using PhEMD

PhEMD was employed to generate a three-dimensional embedding of patients based on their immune cell profiles (38). Briefly, PhEMD first generates a reference map of cell subtypes, then uses Earth Mover's Distance (EMD) to compute pairwise dissimilarities between patients (incorporating patient-to-patient differences in cell fractions of each cell subtype as well as intrinsic dissimilarities between subtypes based on the cell subtype reference map), and finally applies a dimensionality reduction technique to the patient-to-patient distance matrix to generate a final embedding of patients. The Seurat implementation of 3D Uniform Manifold Approximation and Projection (UMAP) was used to map the cell-subtype space using the Harmony batch-corrected components as input and a “min.dist” parameter of 0.4, and cell subtypes (i.e., clusters) were defined as described in the “Data quality control and Normalization” section of Methods (27, 34). Dissimilarity between each pair of cell subtypes was defined as the distance between the centroids (in UMAP space) of all cells assigned to the two respective subtypes. PHATE was applied to the EMD patient-to-patient distance matrix to generate the final 3D embedding of patients (39).

xvi. Luminex Assay for Antibody Titer

Highly immunogenic linear regions of the SARS-COV-2 proteome were isolated by ReScan and conjugated to Luminex beads as previously described (40). Briefly, high concentration T7 phage stocks displaying immunodominant epitopes of the S, N and ORF3a proteins were propagated and grown to high (>1011 PFU/mL) titer then were each conjugated to unique bead IDs according to manufacturer's Antibody Coupling Kit instructions (Luminex). Whole N protein (RayBiotech) beads were conjugated similarly using manufacturer instructions with 5 μg of protein per 1 million beads. For other whole protein Luminex-based beads, MagPlex-Avidin Microspheres (Luminex) were coated with either the S protein RBD (residues 328-533) or the trimeric S protein ectodomain (residues 1-1213). All beads were blocked overnight before use and pooled on day of use. 2000-2500 beads per ID were pooled per incubation with patient serum at a final dilution of 1:500 for 1 hour, washed, then stained with an anti-IgG pre-conjugated to phycoerythrin (Thermo Scientific, #12-4998-82) for 30 minutes at 1:2000. Primary incubations were done in PBST supplemented with 2% nonfat milk and secondary incubations were done in PBST. Beads were processed in 96 well format and analyzed on a Luminex LX 200 cytometer. Median Fluorescence Intensity from each set of beads within each bead ID were retrieved directly from the LX200 and log transformed after normalizing to the mean signal across two intra-assay negative controls (glial fibrillary acidic protein (GFAP) and Tubulin phage peptide conjugated beads).

xvii. Luminex Assay for Serum Cytokines

Soluble proteins were quantified in EDTA anticoagulated plasma using the Luminex® multiplex platform (Luminex, Austin TX) with custom-developed reagents (R&D Systems, Minneapolis, MN), as described in detail (41) or single-plex ELISA (R&D Systems, Minneapolis, MN). Analytes quantified using the Luminex® multiplex platform were read on the MAGPIX® instrument and raw data were analyzed using the xPONENT® software. Analytes quantified using single-plex ELISA were read using optical density. Values outside the lower limit of quantification were assigned a value of ⅓ of the lower limit of the standard curve for analytes quantified by Luminex and ½ of the lower limit of the standard curve for analytes quantified by ELISA.

xviii. O-Link Assay for Serum Factors

Circulating proteins were measured in plasma using the multiplexed Proximity Extension Assay (PEA) from Olink Proteomics AB (Uppsala, Sweden). 20 μL each of plasma collected from the COMET patient cohort (21 COVID-19 positive, 13 COVID-19 negative, and 14 healthy individuals) were analyzed using the Olink® Target 96 Inflammation panel, which is a set of 92 inflammation-related protein biomarkers. Plasma for all samples regardless of COVID-19 status were inactivated using a final concentration of 1% (v/v) Triton-X-100 solution over 2 hours. Data from the analyzed protein biomarkers is presented as Normalized Protein expression (NPX) values, an arbitrary unit on a log 2 scale.

xix. ELISA Method for Serum IFNα Measure

IFN-α levels were quantified from serum by an ELISA (catalog numbers 41115 for IFN-α; PBL Assay Science). ELISA was performed according to the manufacturer's instructions with minor modifications. Briefly, an 8-point standard curve was prepared and diluted in sample buffer. Serum was also diluted by adding 80-90 μl serum to 30 μl sample buffer (depending on availability of serum). Samples were prepared in duplicates, whereas standards were prepared in triplicates. Initial incubation was performed for 20 hours at 4° C. Antibody was added and incubated at 4° C. overnight. HRP was added and incubated 1 hr at room temperature, TMB substrate was added for 30 min and incubated in the dark at room temp, stop solution was added and samples were read using a SpectraMax M2 Microplate Reader (Molecular Devices) at 450 nm. For analysis, a 4-parameter logistic fit was applied to OD values of the standards after background subtraction. Samples with ODs below blank samples were considered as 0 μg/ml IFN-α.

xx. Pbmc Co-Culture Experiment with Patient Serum and Flow Cytometry Analysis

PBMCs were isolated from EDTA-anticoagulated whole blood from healthy donors using Polymorphprep (Alere Technologies), and resuspended in culture medium (RPMI 1640+10% FBS). For detection of neutralization of interferon stimulation, autologous serum or clinical study participant sera (10 μl) were plated with IFNα (Stemcell IFN alpha-2A; final concentration of 1 pg/μl) in a total volume of 200 μl before addition of 2.5×10⁵ PBMCs. After incubation for 24 hours, PBMCs were assayed for IFNα-induced IFITM3 upregulation and CD14/CD16 levels and fractions by flow cytometry. After surface staining and addition of fixable live/dead violet dye (ThermoFisher; #L34955), intracellular detection of IFITM3 was done using the eBioscience Foxp3/Transcription Factor Staining Buffer Set (ThermoFisher; #00-5523-00) and following the manufacturer's instructions. For FcR blocking experiments, Fc receptors were blocked with unconjugated anti-CD16 (clone 3G8; BioLegend; #302002), anti-CD32 (clone FUN-2; BioLegend; #303202), anti-CD64 (clone 10.1; BioLegend; #305002), anti-CD32a (Clone IV.4, BioXcell) and anti-CD32b/c (clone S18005H Biolegend) with 0.5 μg of each antibody. After incubation for 24 hours with IFNα (1 pg/μl), PBMCs were assayed for IFNα-induced IFITM3 upregulation and CD14/CD16 levels and fractions by flow cytometry. For serum staining assays, PBMCs were cultured with media or 1-100 μg/ml IFNα for 38-46 hours. Samples were harvested and Fc receptors were blocked with unconjugated anti-CD16 (clone 3G8; BioLegend; #302002), anti-CD32 (clone FUN-2; BioLegend; #303202), and anti-CD64 (clone 10.1; BioLegend; #305002) antibodies for 20 min on ice. Following one washing step with fluorescence-activated cell sorting (FACS) buffer (2% fetal bovine serum, 1 mM EDTA, PBS), non-specific binding of the detection antibody was blocked by incubating with unconjugated AffiniPure Donkey anti-human IgG (Jackson Immunoresearch; #709-005-149) for 15 min at room temperature. After washing with FACS buffer, PBMCs were then stained for surface markers 30 min on ice. After staining incubation, cells were washed 3× times with FACS buffer (1500 rpm, 5 min, 4° C.) and incubated with 5 μl autologous or clinical study participant sera for 30 min on ice. After washing the cells with FACS buffer, cell-bound antibodies were detected using an AffiniPure Donkey anti-human IgG-Alexa Fluor 647 antibody (Jackson Immunoresearch; #709-605-149), which was incubated with the cells for 30 min on ice. Cells were washed again and resuspended in 1 μg/ml DAPI solution for live/dead discrimination. The following antibodies were used for flow cytometric analysis: anti-human CD3-BB700 (clone SK7; BD Biosciences; #566575), anti-human CD14-BV711 (clone MSE2; BioLegend; #301838), anti-human CD15-BV786 (clone W6D3; BD Biosciences; #741013), anti-human CD16-BV605 (clone 3G8; BioLegend; #302040), anti-human CD19-BV785 (clone HIB19; BioLegend; #302240), anti-human CD45-APCeFluor780 (clone HI30; ThermoFisher; 47-0459-42), anti-human IFITM3-AlexaFluor 647 (clone EPR5242; Abcam; ab198573).

xxi. PBMC Fc Receptor Crosslinking Experiment

96 well flat bottom polystyrene plates were coated overnight at 4° C. with either 10 or 5 μg/mL of combinations of anti-CD16 (clone 3G8; BioLegend; #302002), anti-CD32 (clone FUN-2; BioLegend; #303202), and anti-CD64 (clone 10.1; BioLegend; #305002) diluted in PBS. Plates were washed 3× with PBS prior to PBMC plating which were prepared as detailed above. 250 k PBMC's per well were spun down briefly and incubated at 37° C. for 15 minutes to allow for coated antibody engagement. IFNα was then added into the well and cells incubated for 24 hours at 37° C. prior to flow cytometry as described above.

xxii. Bead ELISA

10⁷ 5 μm Sulfate latex polystyrene beads (Thermo Fisher) were resuspended in 1 ml of PBS to which 1 μg of proteins (BSA, huIFNα (Stemcell IFN alpha-2A) were added to bind by passive absorption over 1 hour on ice. Beads were washed 1× in PBS and blocked with 1 ml of blocking buffer (PBS containing 1 mM EDTA and 2% FCS) for one hour. Beads were spun and resuspended in 1 ml of blocking buffer and 10 μl (105 beads) were moved to individual tubes to which 5 μl of sera was added followed by incubation for 1 hour on ice). These were washed, resuspended in 50 μl of blocking buffer containing Goat anti-human IgG-Alexa Fluor 647 (Jackson Immunoresearch) and incubated for 1 hr on ice followed by a final wash and analysis by flow cytometry.

xxiii. RNA Extraction and qPCR

RNA extraction from healthy PBMCs treated with IFN-α with the addition of Mild/Moderate or Severe patient sera and with or without Fc receptor blocking was performed using a Qiagen RNEasy Plus Micro Kit (Qiagen). cDNA was synthesized from extracted RNA using a High Capacity cDNA Reverse Transcription Kit (Applied Biosystems). qPCR analysis was performed using the SsoFast EvaGreen Supermix (BioRad) according to manufacturer's protocols on a BioRad CFX96 Touch Real-Time PCR Detection system. Fold changes were calculated relative to untreated healthy PBMCs and using B-actin as the internal control. In addition, the fold induction in healthy PBMCs treated with IFN-α treatment (relative to untreated) was used to normalize plate-by-plate variability. Primers for detecting ISG have been previously described here (protocolexchange.researchsquare.com/article/nprot-6391/v1).

xxiv. SARS-COV-2 Detection by PCR

PCR testing for SARS-COV-2 was carried out on respiratory specimens mixed 1:1 in DNA/RNA Shield (Zymo Inc.) using an in-house Clinical Laboratory Improvement Amendments (CLIA) validated assay at the UCSF Clinical Microbiology Laboratory. PCR primers targeted the SARS-COV-2 envelope (E) and RNA-dependent RNA polymerase (RdRp) genes plus human RNAse P as a positive control.

xxv. Statistical Analysis and Data Visualization

Statistical analyses were performed using GraphPad prism or the R software package. Null hypotheses between two groups were tested using the non-parametric Mann-Whitney test to account for non-normal distribution of the data. Likewise, for multiple groups, comparisons were made by two-way ANOVA or non-parametric Kruskal-Wallis test followed by multiple comparisons. The specific statistical tests and their resultant significance levels are also noted in each figure legend. The R packages Seurat, ggplot2 (version 3.1.0) (Wickham, 2016) GraphPad Prism and Adobe Illustrator were used to generate figures.

xxvi. Data Resources and Code Sharing

Cellranger-processed raw feature-barcode matrices are available at GEO using accession GSE163668 and raw fastq files for all 10× libraries are deposited in SRA. Scripts used to process all data along with relevant clinical information for each patient are available at github.com/UCSF-DSCOLAB/combes_et_al_COVID_2020.

### 2. Results

To understand immune biology amongst COVID-19 patients, we compared them to patients presenting with similar respiratory symptoms but who were not infected with the SARS-CoV-2 virus. We enrolled 21 SARS-COV-2 positive inpatients, 11 inpatients with similar clinical presentations consistent with acute lung injury (ALI) or acute respiratory distress syndrome (ARDS), who were SARS-COV-2 negative-those caused by other infections or of unknown origin—and 14 control individuals. We further categorized these individuals as “mild/moderate” (M/M: short stays in hospital with no need for mechanical ventilation and intensive care) or “severe” (requiring intubation and intensive care) according to the full clinical course of their disease (FIG. 1A, FIG. 5A, and Table 2). Hence, our study includes patients with mild/moderate (n=11) or severe (n=10) COVID-19 and patients with mild/moderate (n=6) or severe (n=5) non-COVID-19 ALIARDS. With the exception of one individual, all our patients who presented with mild/moderate disease remained mild/moderate during hospitalization (FIG. 5A), suggesting that mild/moderate and severe are more stable states rather than transient phases of disease in this cohort.

Since the majority of COVID-19 mortality is among patients with ARDS—characterized by an exuberant immune response with prominent contributions from neutrophils, monocytes, platelets—we focused upon collecting these cells along with other major populations. We thus performed single-cell RNA sequencing (scRNA-seq) on RBC-depleted blood samples from all individuals. After merging, batch-correction and doublet-removal, our data comprised 116,517 cells (FIG. 1B, FIG. 5B) among which we identified neutrophils, platelets, mononuclear phagocytes, T/NK cells, B cells, plasma cells and eosinophils (FIG. 1B, FIG. 5C). We confirmed a positive association between neutrophil frequency and disease severity and an inverse correlation for lymphoid populations (FIG. 1B, FIG. 5D) (1-3). At this level of resolution, findings were similar between SARS-COV-2 negative and positive individuals (FIG. 5F).

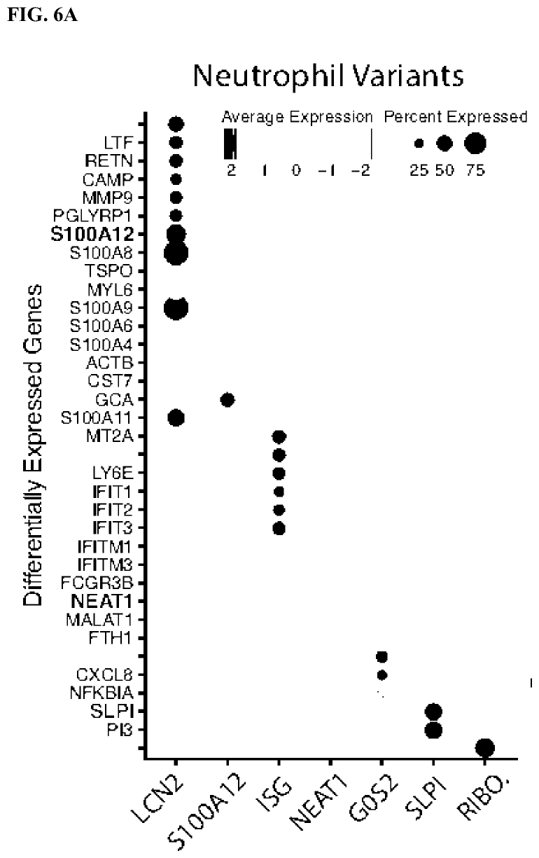

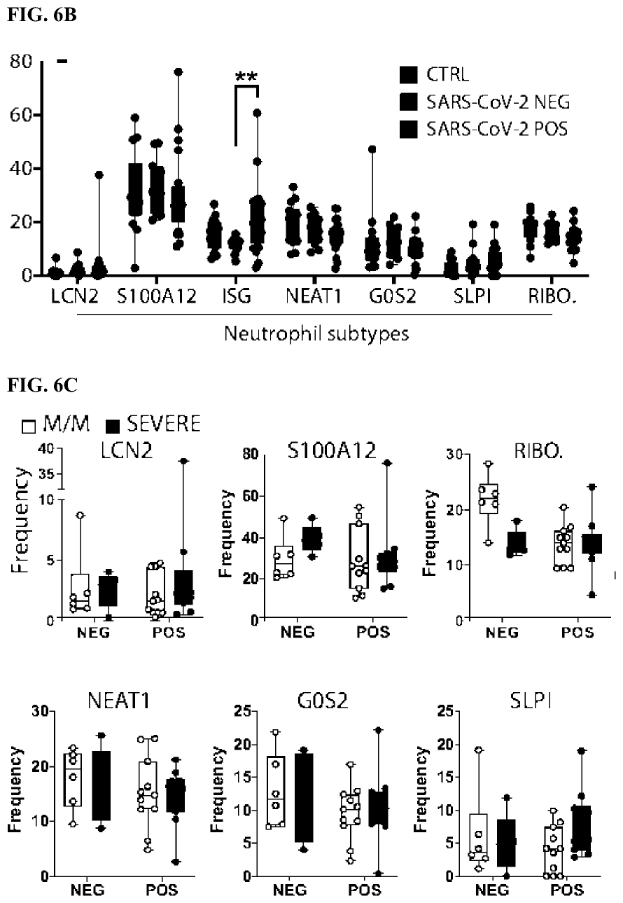

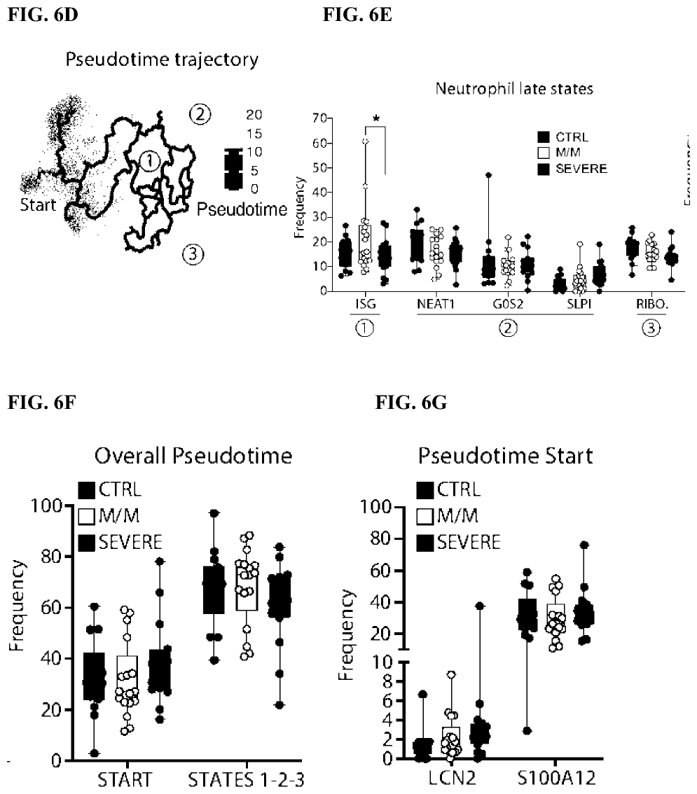

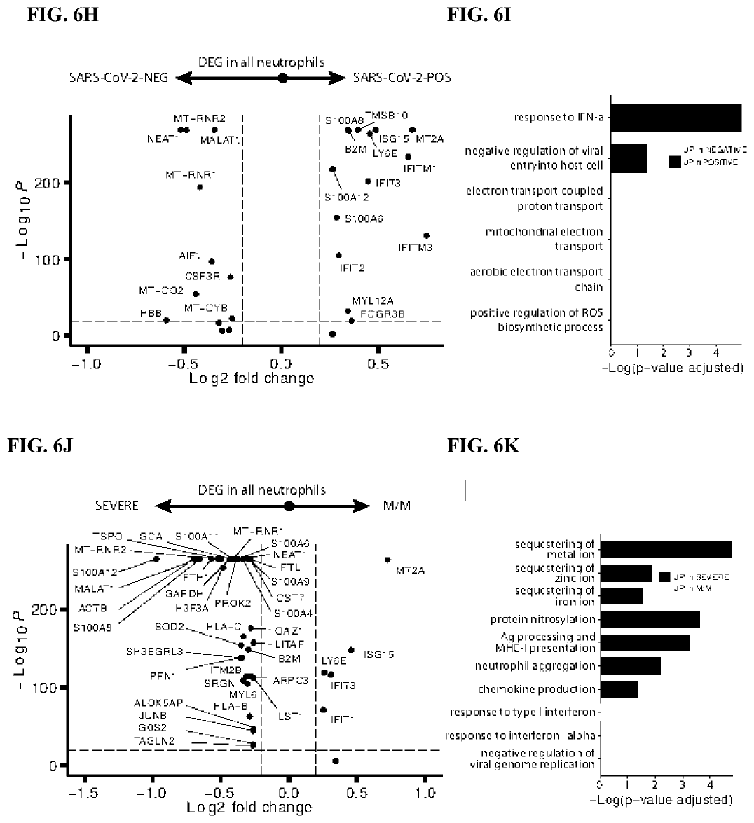

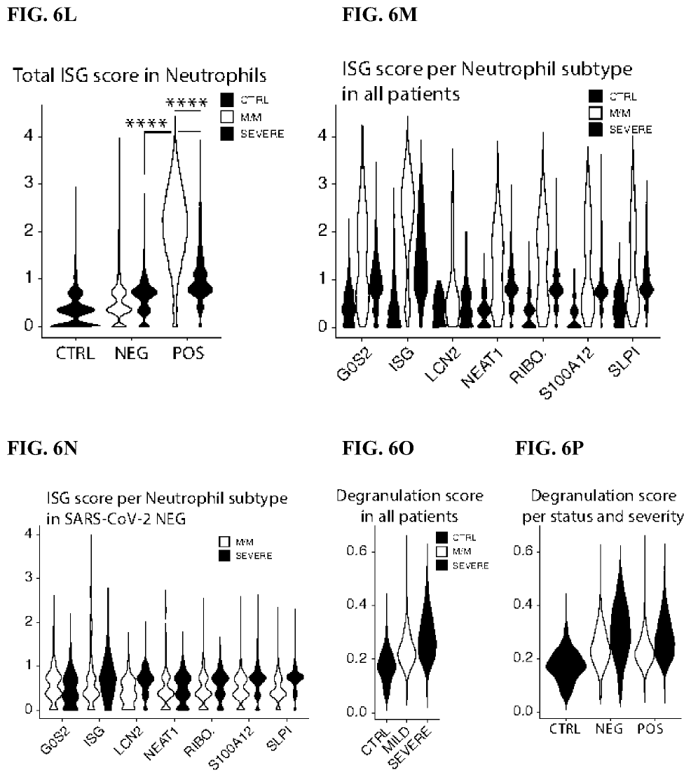

Within the neutrophils, we identified seven subtypes (FIG. 1C, FIG. 5A), consistent with previous studies (2, 4). One population, harboring a strong interferon-stimulated gene (ISG) signature and henceforth termed ISG neutrophils, was highly enriched in SARS-COV-2 positive patients but not in those whose disease was severe (FIG. 1D-1E, FIG. 6B). Separate pseudotime analysis (FIG. 6D-G) placed the ISG subtype as a late-stage of differentiation and was the only such state found significantly altered between mild/moderate and severe patients (FIG. 6E) and specifically within the SARS-COV-2 positive individuals (FIG. 1F, FIG. 6C). ISG signature genes include master anti-viral regulators such as ISG15 and IFITM3 which restricts viral entry into the cytosol (6).

We also analyzed differentially expressed genes (DEG) from SARS-COV-2 positive versus negative patients, and from mild/moderate versus severe patients across all neutrophils. ISG signature genes were expressed differentially higher in all neutrophil subsets, specifically in SARS-COV-2 positive mild/moderate patients, as compared to SARS-COV-2 positive severe patients (FIG. 1G, FIG. 6H-6N). In contrast, a separate neutrophil degranulation gene program is upregulated in neutrophils from mild/moderate patients as compared to severe regardless of COVID status (FIG. 6O-6P). This suggests a shared program of degranulation enhancement in all respiratory infections regardless of causative pathogen, and a global induction of the ISG program in all neutrophils in mild/moderate SARS-COV-2 positive patients that is absent in severe ones (3).

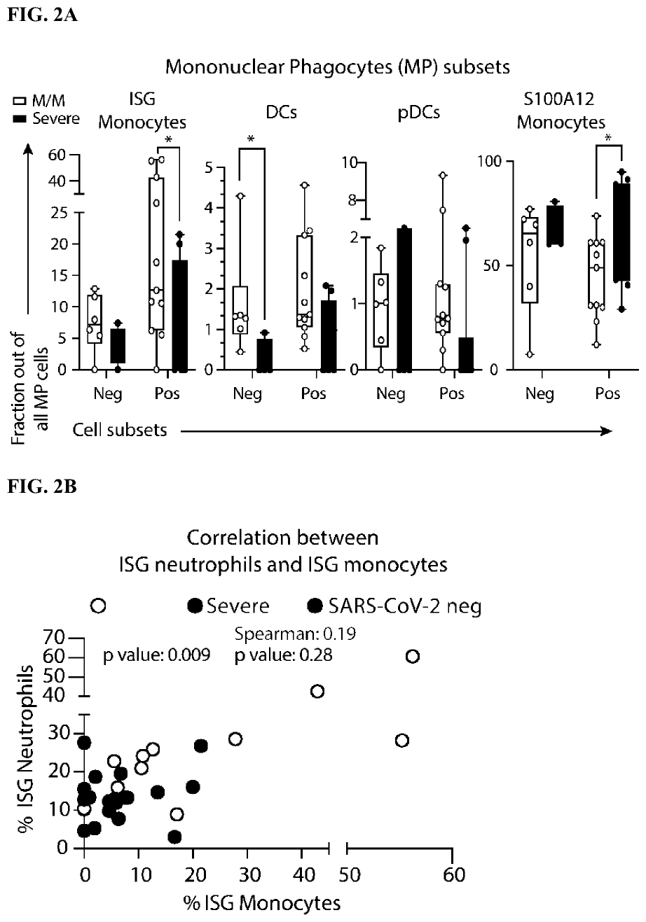

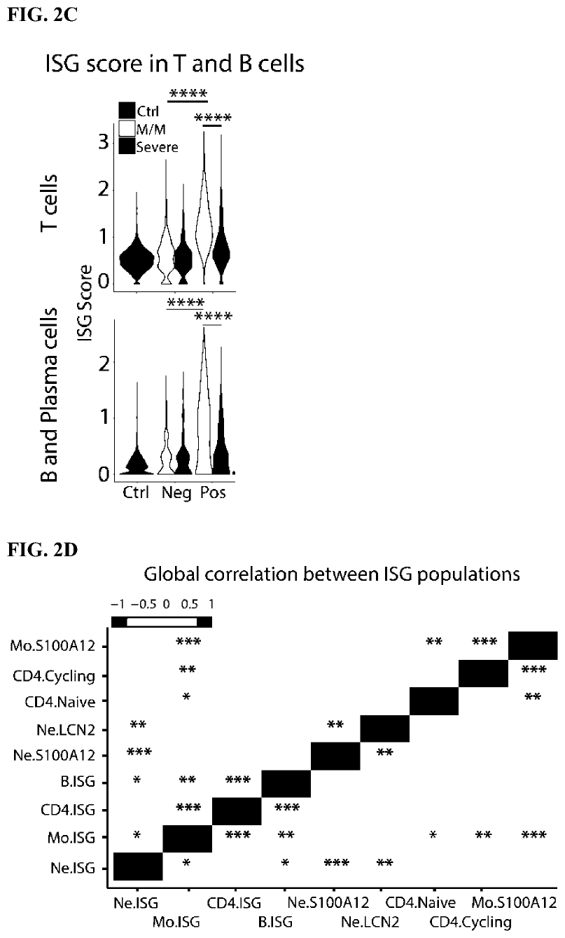

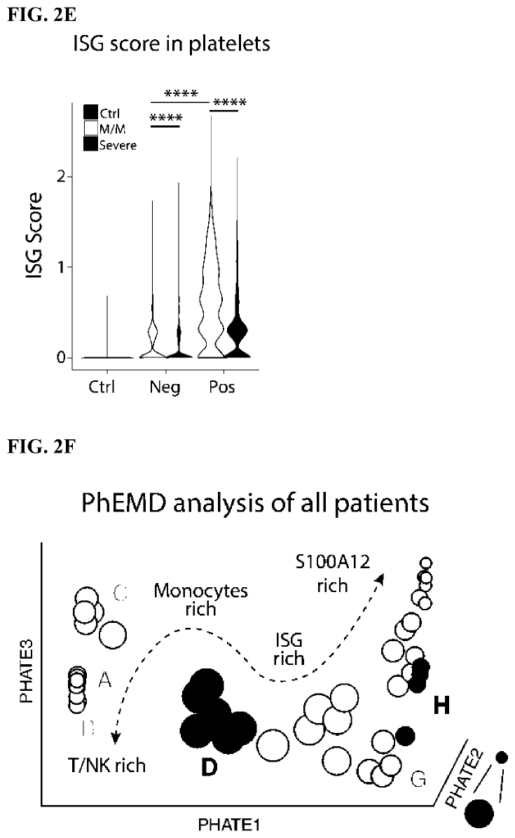

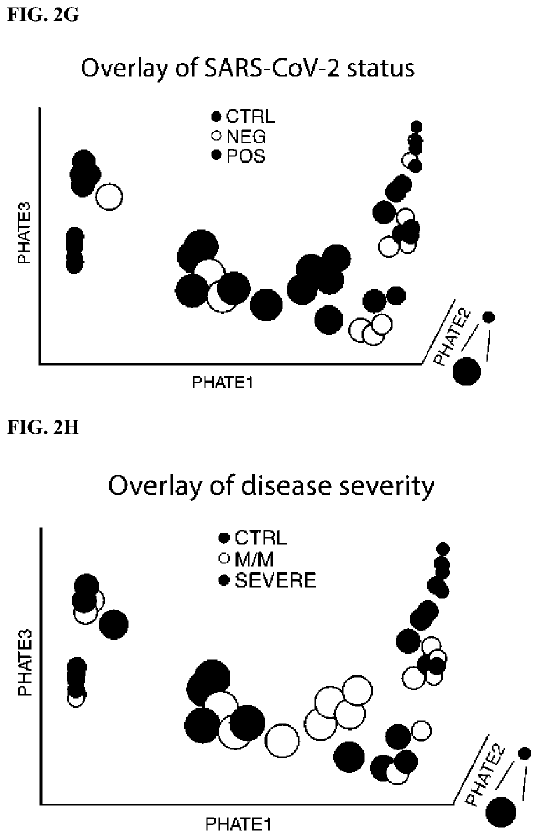

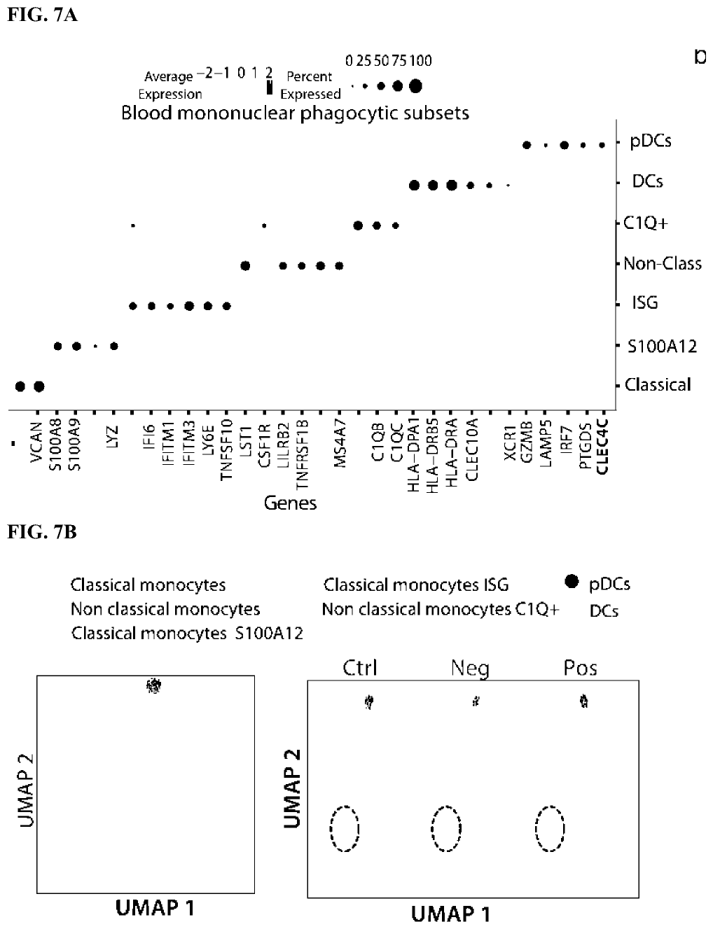

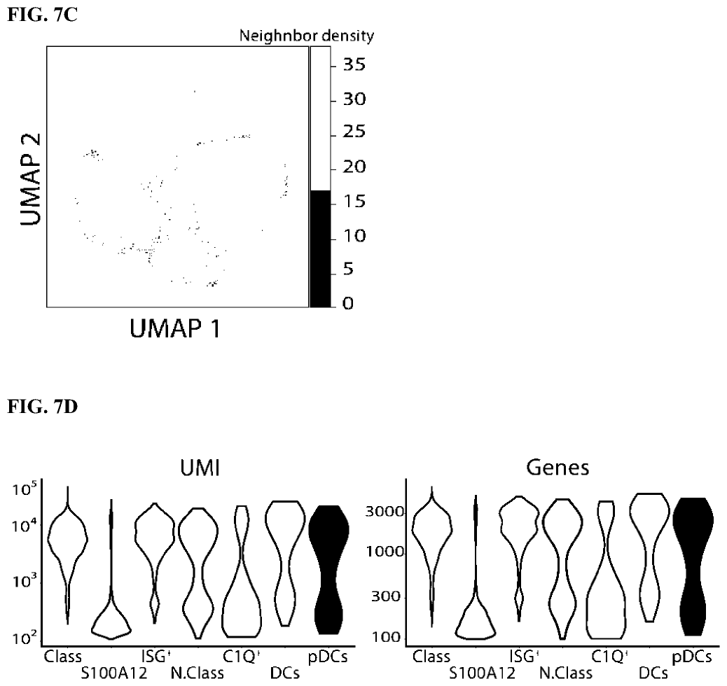

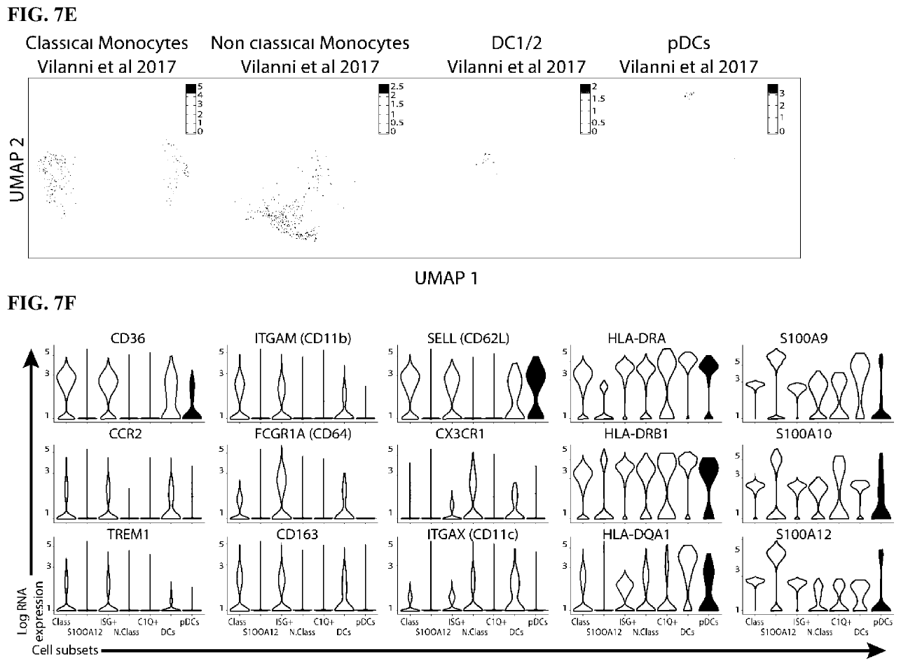

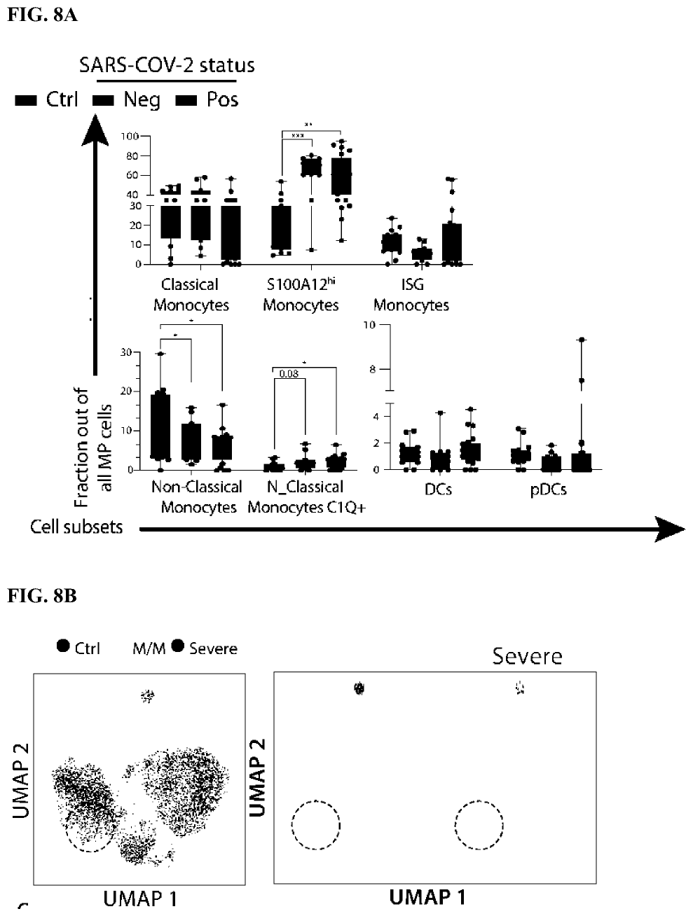

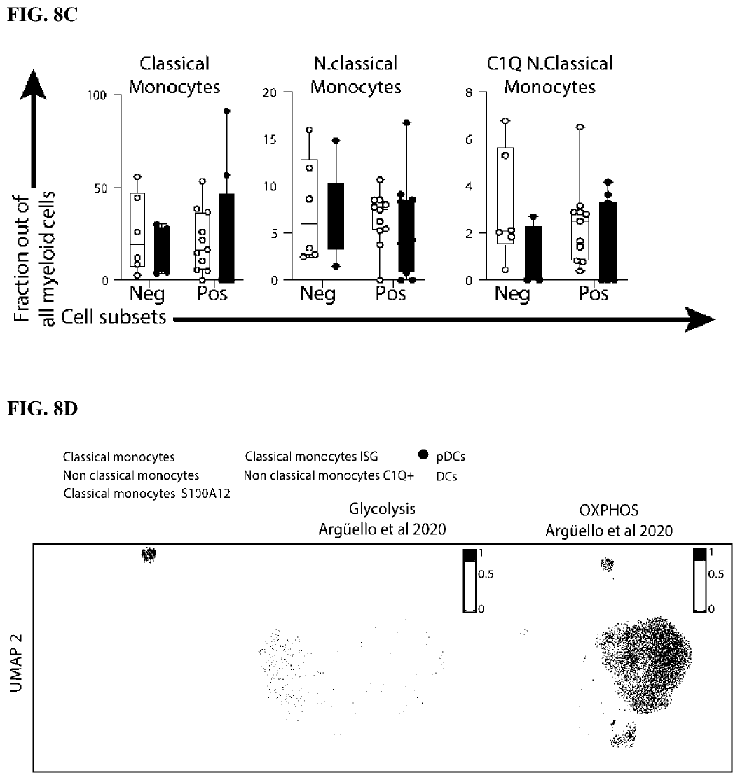

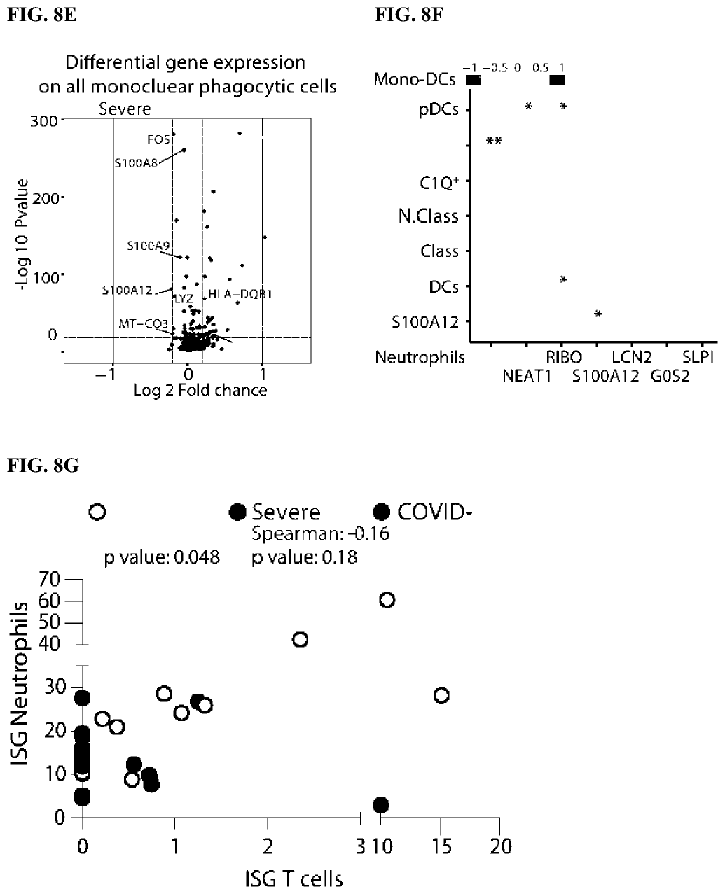

Assessing the mononuclear phagocytes—monocytes, macrophages, dendritic cells and plasmacytoid dendritic cells (pDC)—yielded 7 clusters of transcriptionally distinct cells subsets, evenly distributed across our cohort (FIG. 7A-7F). We identified an ISG expressing classical monocytes cluster as being enriched in SARS-COV-2 positive patients, and particularly those having mild/moderate disease, similarly to neutrophils (FIG. 2A, FIG. 8A-8C). ISG monocytes also expressed genes associated with glycolysis, compared to a S100A12-expressing subset that were enriched for genes associated with oxidative phosphorylation, consistent with previous reports in bacterial sepsis (7) (FIG. 8D). DEG analysis demonstrated that ISGs were the dominant genes associated with mild/moderate phenotypes when the entire mononuclear phagocyte pool was assessed (FIG. 8E).

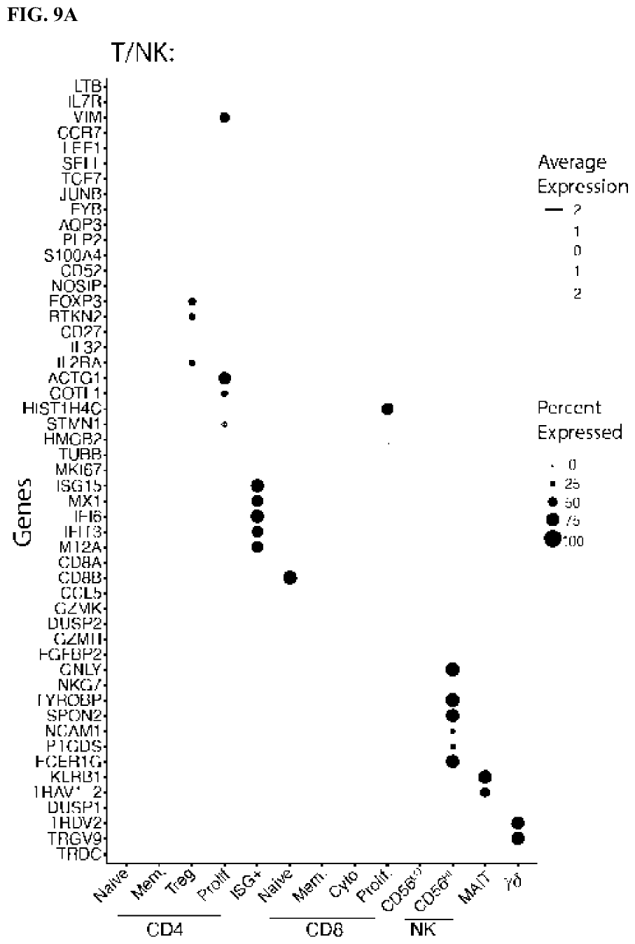

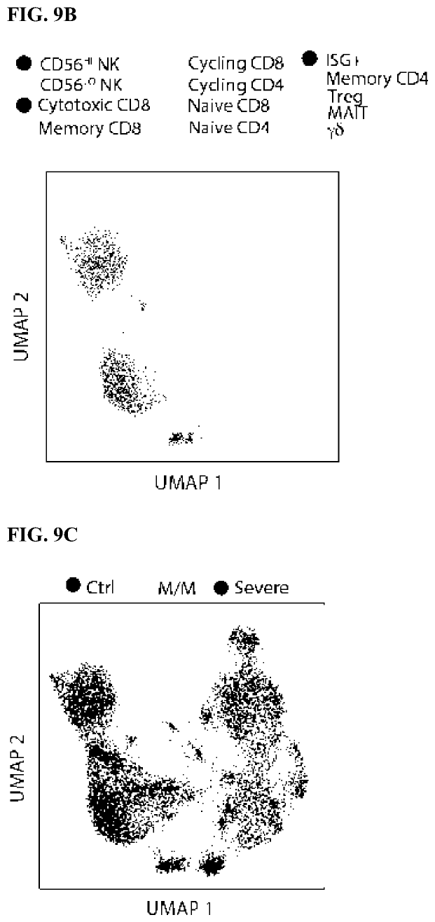

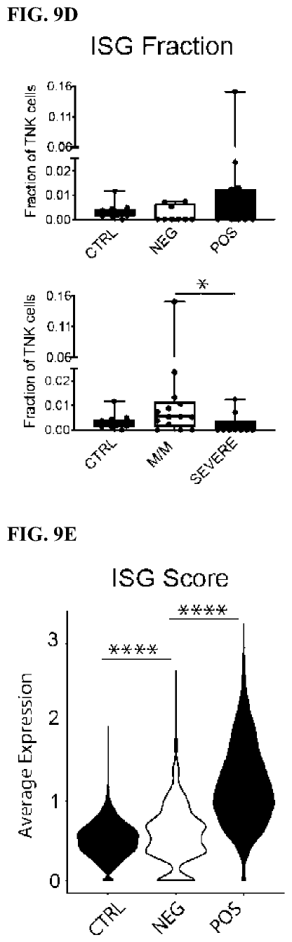

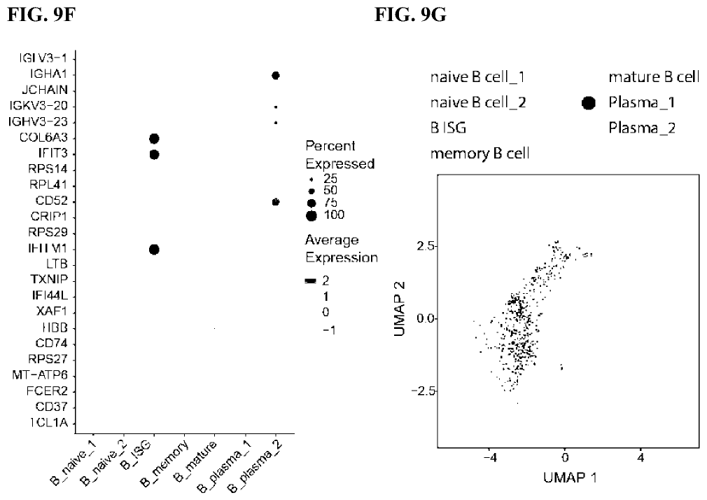

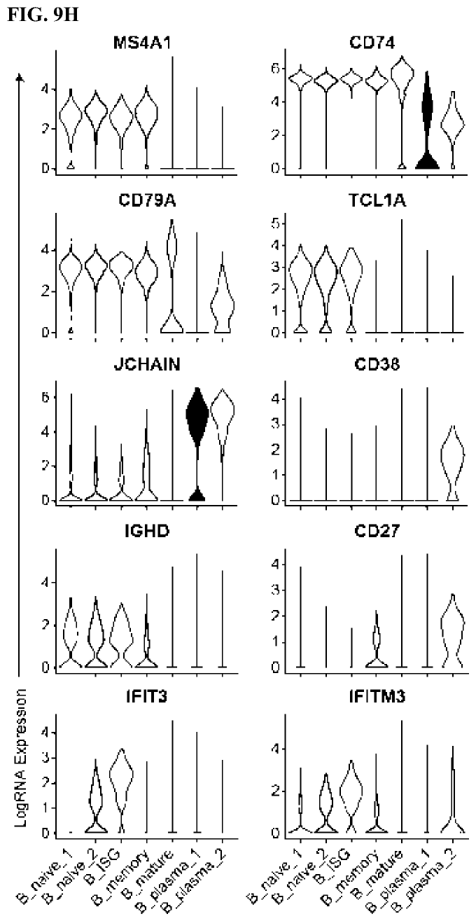

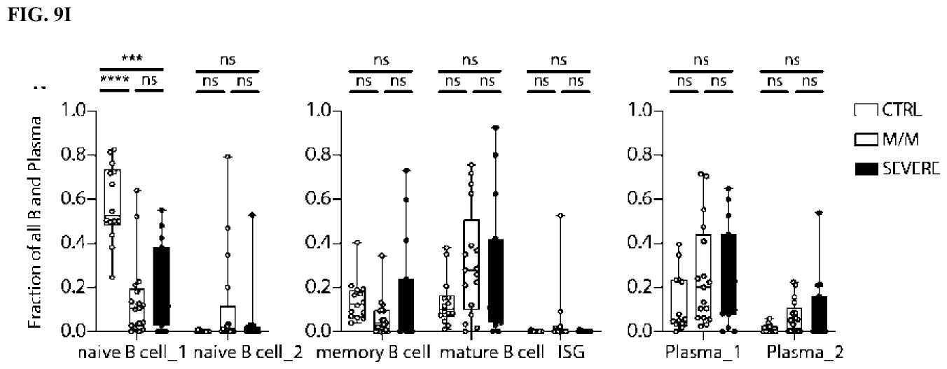

ISG monocytes and ISG neutrophils frequencies were strongly correlated with one another in mild/moderate SARS-COV-2 positive individuals (FIG. 2B, FIG. 8F). A comprehensive analysis of T cell and B cell frequencies (FIG. 9) demonstrated that both cell types were also significantly enriched in ISG signatures, specifically in mild/moderate COVID-19 patients (FIG. 2C). The frequencies of ISG+ cells in one compartment correlated with the frequency of ISG expressing cells in another, for example ISG+ T cells and ISG+ neutrophils, uniquely in mild/moderate patients (FIG. 8G). Spearman correlation analysis across multiple cell types in all patients thus showed a collection of correlated ISG+ populations and a second anti-correlated block of other cell populations, notably those expressing S100A12 (FIG. 2D).

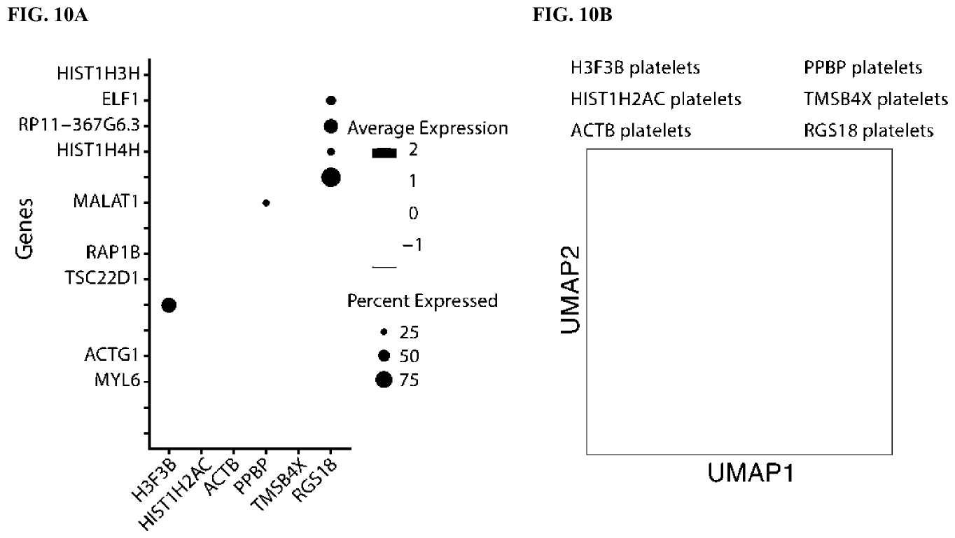

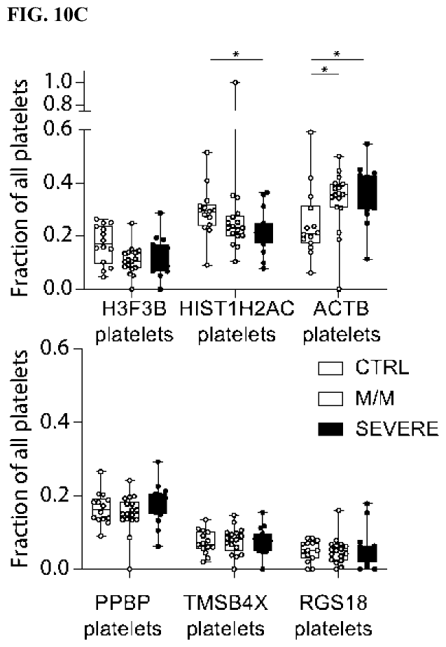

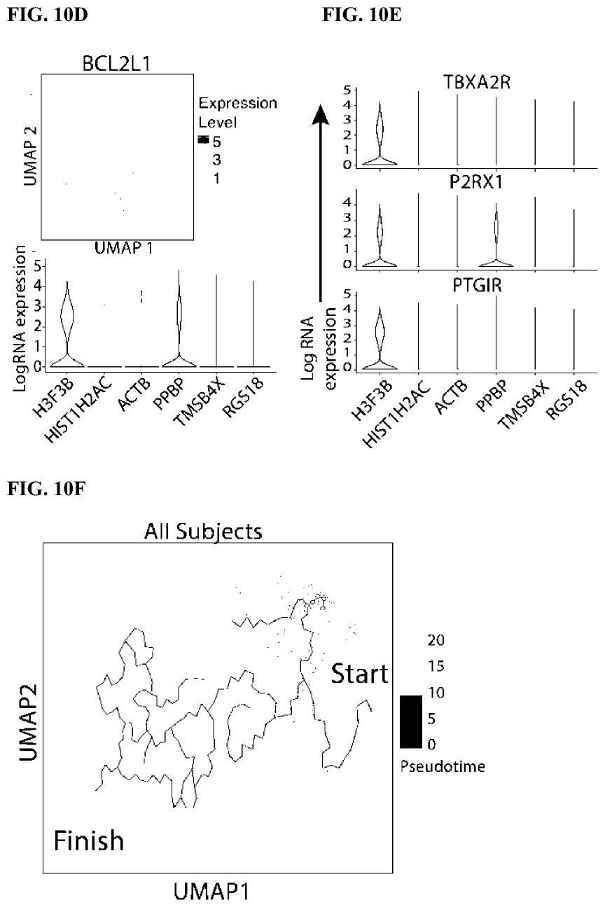

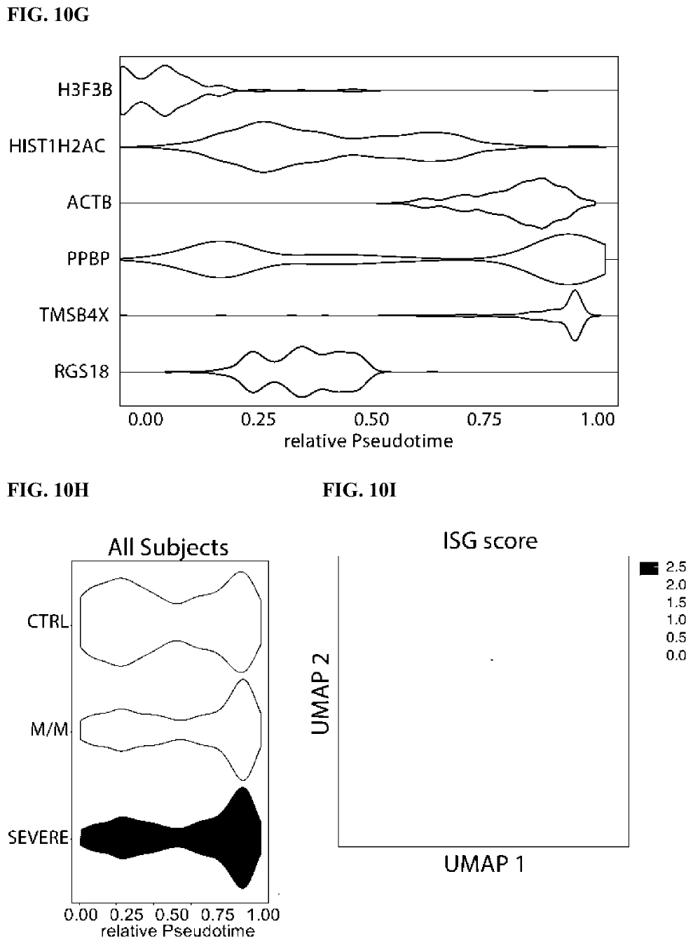

Our scRNA-seq whole blood data set also allowed us to identify platelets and subset them based on established platelet signature genes (FIG. 1A, FIG. 5D). Analysis of these found six clusters, including three (“H3F3B,” “HIST1H2AC,” and “RGS18”) still carrying transcripts acquired from parental cells, megakaryocytes (FIG. 10A-10B) (10). “HISTIH2AC” subset was only modestly depleted in severe COVID-19 patients suggesting a skewing away from “younger” cells (FIG. 10C). This was supported when overlaying the expression of BCL2L1 onto our dataset, which has been identified as a “molecular clock” for platelet lifetime (11). This identified a histone-rich H3F3B cluster as representing “young” platelets (FIG. 10D), a result supported by a second signature of transcripts in young, reticulated platelets (12) (FIG. 10E). Pseudotime analysis rooted at this H3F3B (FIG. 10F-10G) suggested again that platelets from all patients with disease were broadly overrepresented at the end of the trajectory (FIG. 10H). While we did not identify a distinct ISG+cluster (FIG. 10I), akin to myeloid and lymphoid cells, ISG signature scores in platelets from mild/moderate patients was increased relative to severe patients, particularly for SARS-COV-2 infected patients (FIG. 2E).

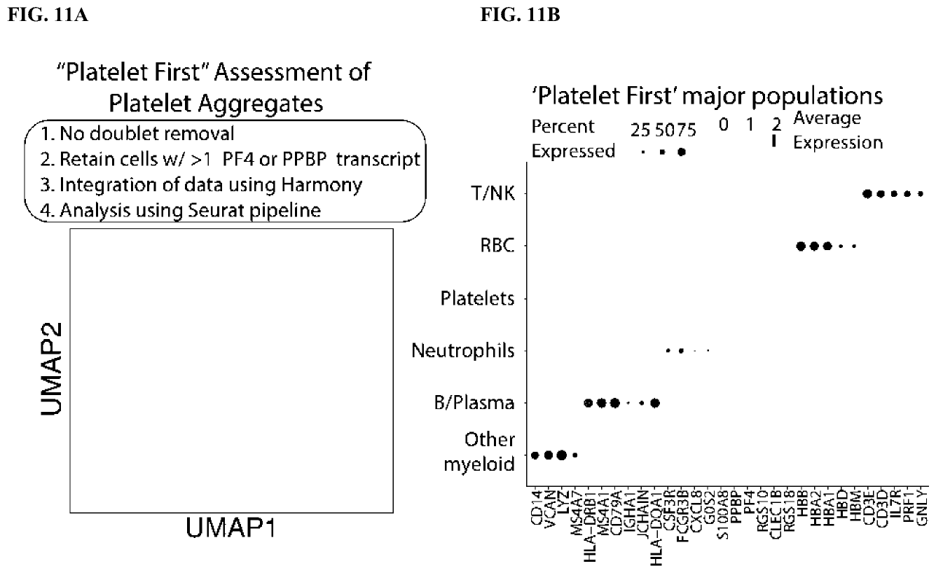

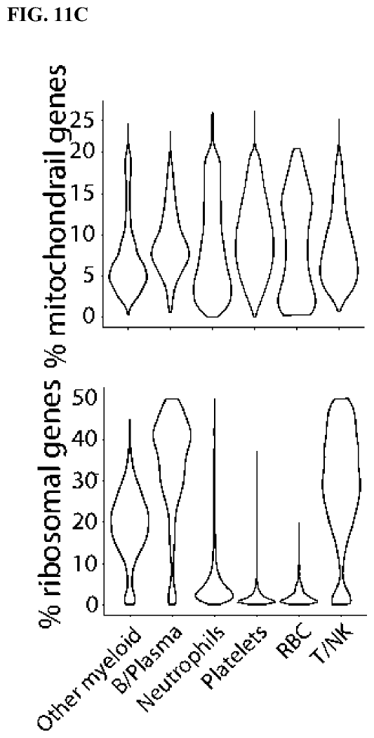

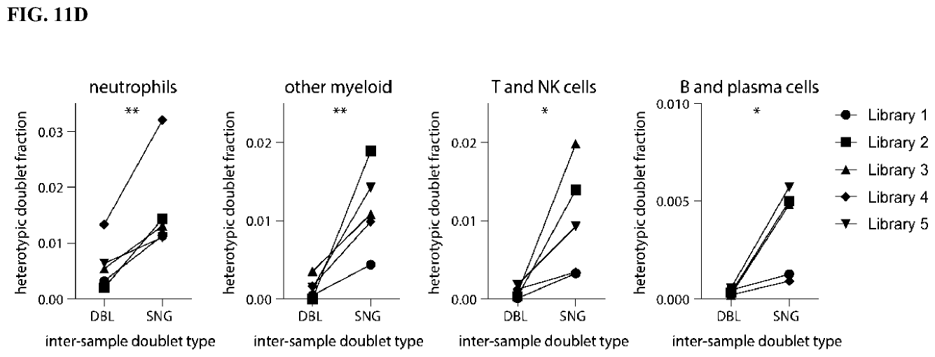

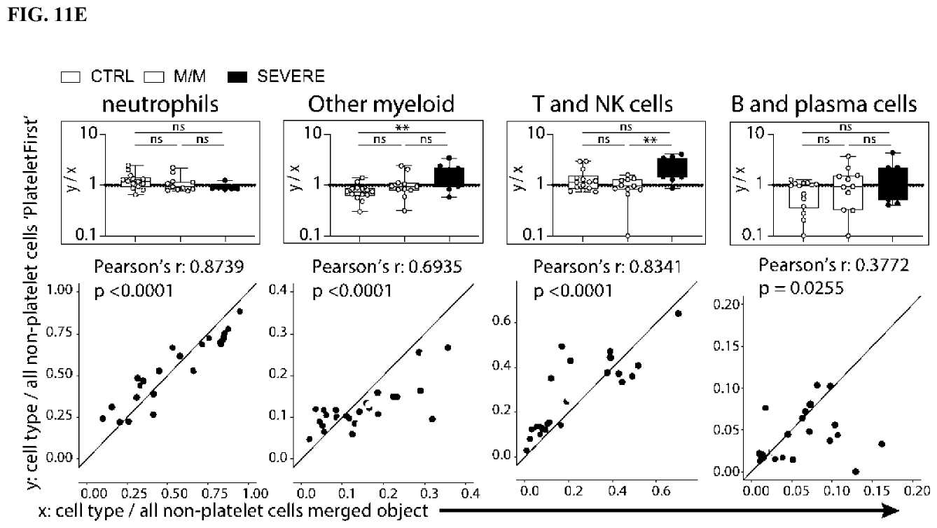

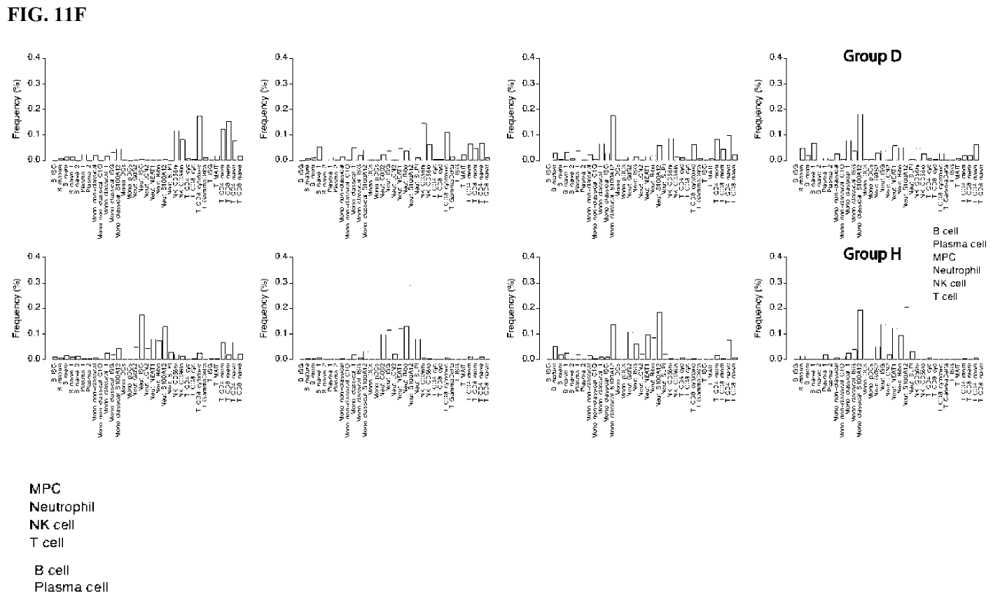

Platelet scRNA-seq also permitted the identification of heterotypic aggregates between platelets and non-platelets by using a “Platelet First” approach (FIG. 11A-11C). This approach revealed the presence of platelet transcripts associated with cells that also bore signatures of other major blood cell types (FIG. 11A-11C). We found no profound differences in frequencies of cell types in this ‘Platelet First’ object compared to the original data set (FIG. 11E). This suggests that, at least in circulating blood, platelets form aggregates indiscriminately with varying other cell types without favoring one or the other.

After observing that ISG expression profiles were elevated in every cell type among patients with mild/moderate disease but globally reduced with severe illness, we turned to a holistic view of disease states. Phenotypic earth mover's distance (PhEMD) (13) embedding of patients based on their subtype frequencies revealed eight distinct groups of patients (FIG. 2F, FIG. 11F) wherein progression from A through H represent patients with generally increasing relative frequency of neutrophils. Intermediates C, D, G and H include patients with relative enrichment in monocytes and E represents patients with an enrichment of ISG neutrophils and mostly consists of SARS-COV-2 positive patients with mild/moderate disease (FIG. 2G-2H). In contrast, Group G, which is an alternative and “severe” fate for patients is highly enriched for neutrophils and has a dominance of S100A12 versus ISG neutrophils (FIG. 11F).

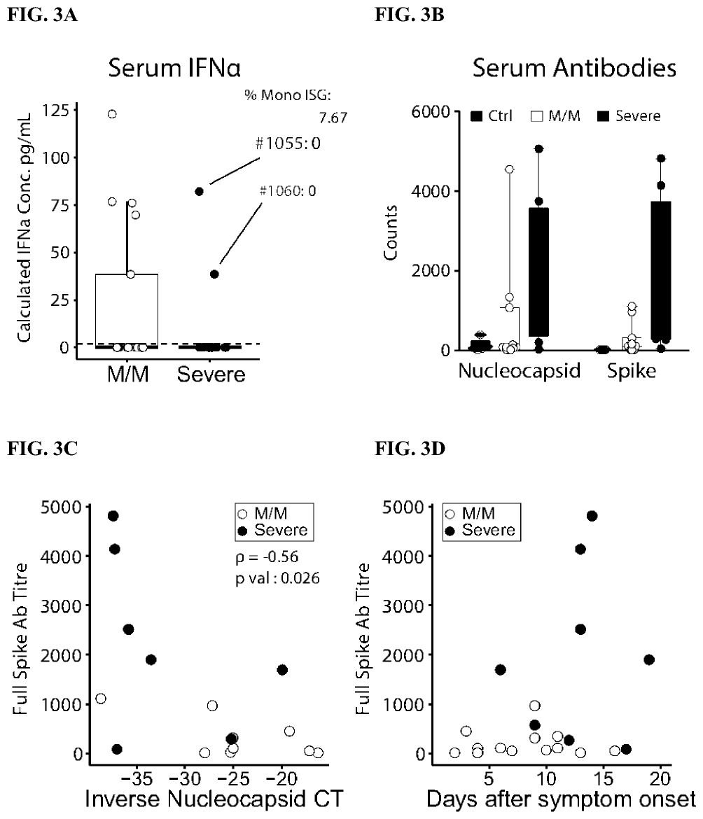

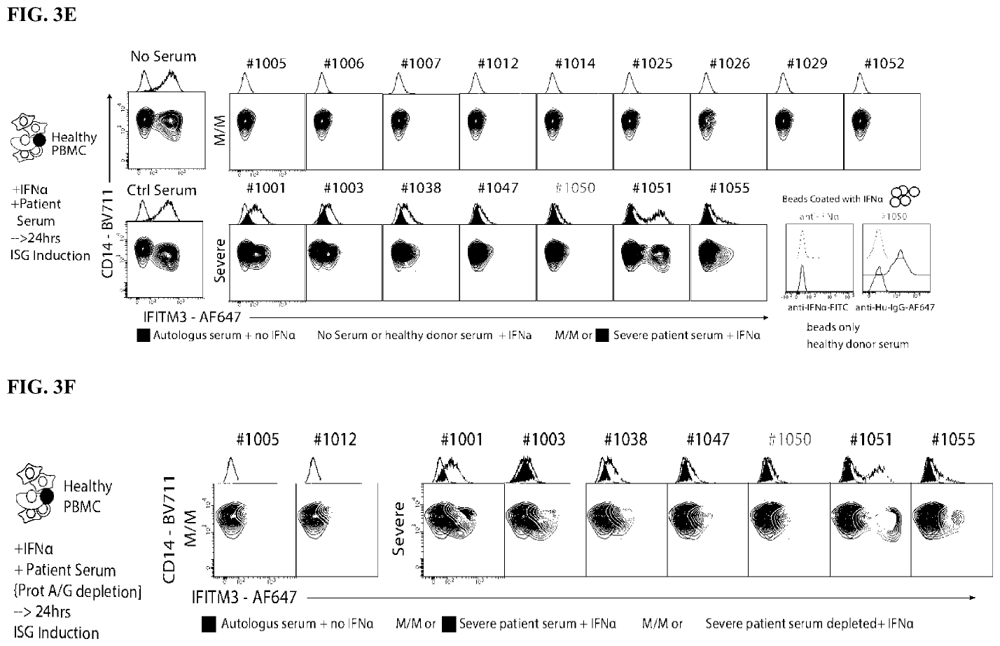

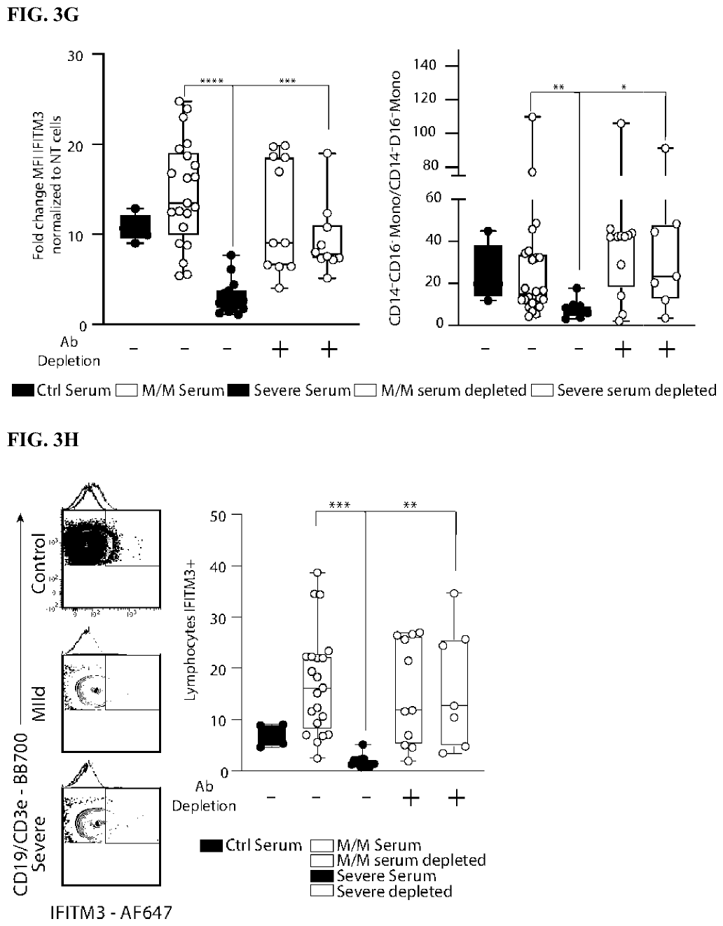

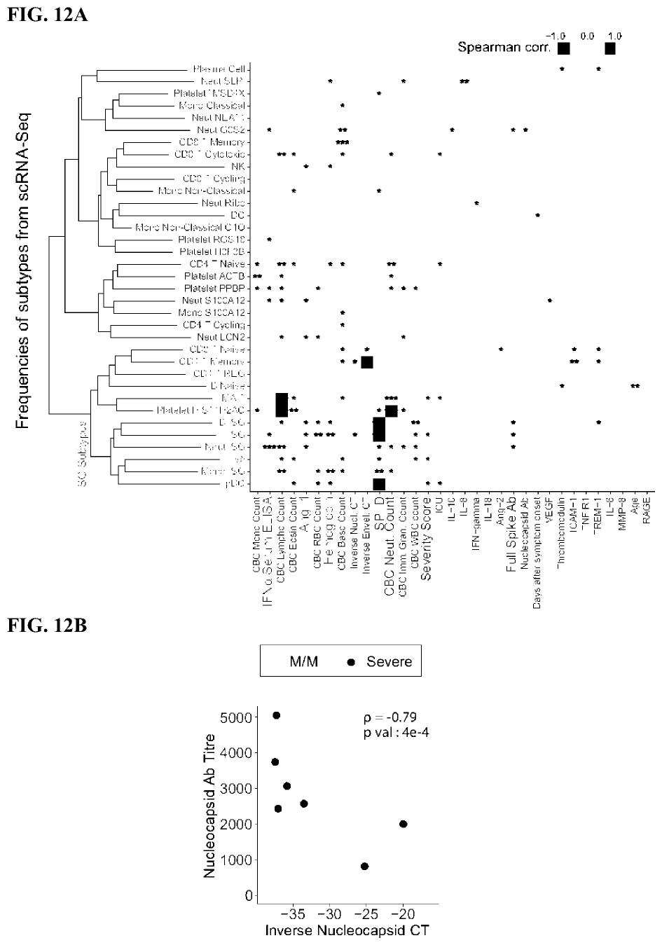

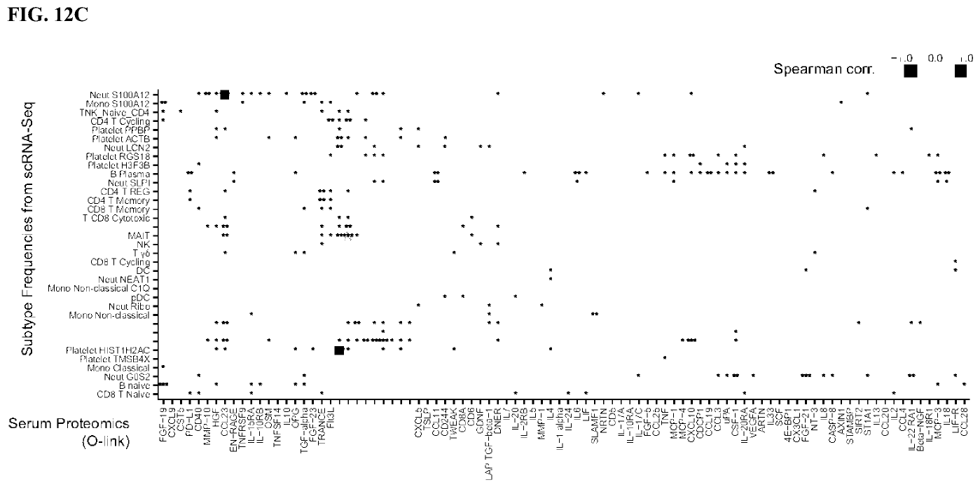

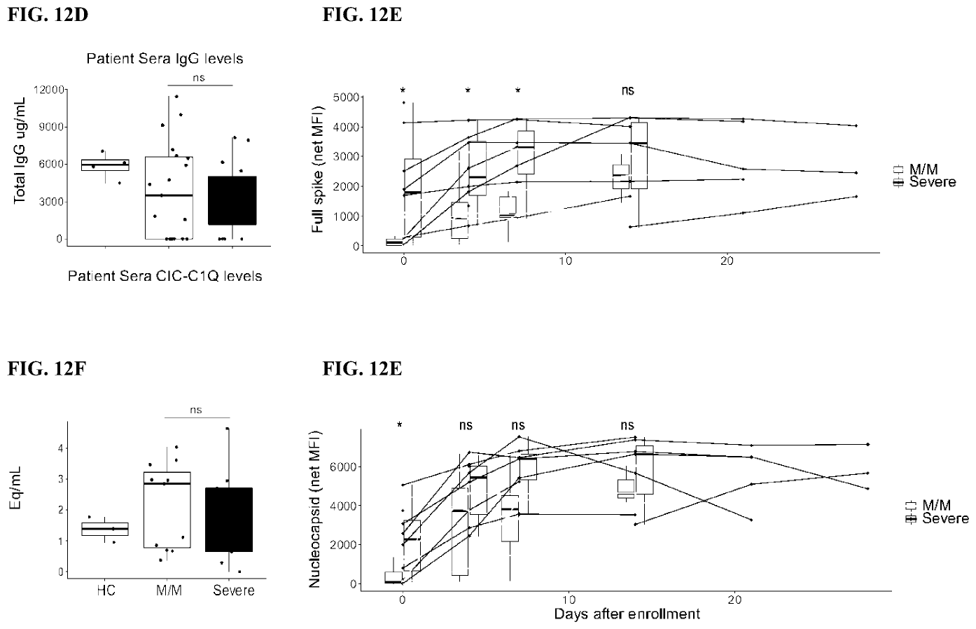

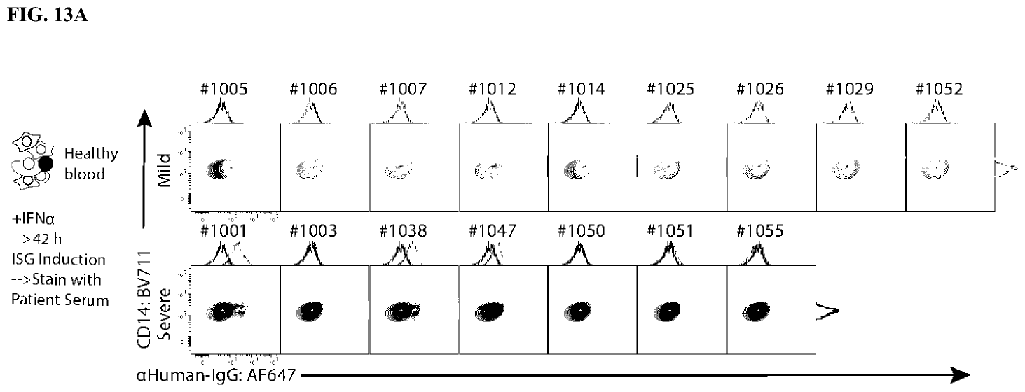

Examination of serum IFNα levels could not explain this loss of ISG+ cell populations in severe patients since severe patients were found with substantial IFNα production (FIG. 3A). However, ISG populations were strongly correlated with low severity of COVID-19 illness, with serum IFN concentration and lower plasma levels of SP-D (indicative of alveolar epithelial injury) (FIG. 12A). When compared to a high-dimensional panel of plasma protein levels (FIG. 12C) most ISG subtypes clustered together and correlated with factors indicative of a strong ISG and Th1 response (CXCL1/6/10/11, TNFB, IL-12B, MCP-2/4). An unexpected anticorrelate of the ISG state was the concentration of serum antibodies against the SARS-COV-2 Spike and Nucleocapsid proteins (FIG. 3B, FIG. 12B).

This anticorrelation was profound and not strongly mirrored in higher total levels of IgG antibodies or immune complexes in severe patient sera (FIG. 12D-12F). We considered it a paradox that severe patients have higher levels of potentially neutralizing antibodies. This is in apparent contradiction with a previous report showing that viral load is associated with severity and mortality in COVID-19 (14, 15), a difference which could be explained by the fact that these studies compare amongst patients with high mortality, which was a very rare event in our cohort (Table 2). At day of admission, both antibody specificities were anticorrelated with the viral load as assessed from nasal swabs (FIG. 3C, FIG. 12B) consistent with though not definitive for being neutralizing. As increased antibody titers and decreased viral load have been reported to be a feature of later disease stage (16), we considered the hypothesis that our observed mild/moderate disease simply preceded severe disease. However, antibody titers in severe patients are consistently higher compared to mild/moderate patients over time, even two weeks beyond symptom onset (FIG. 3D, FIG. 12E), and only one of our 19 mild/moderate patients would go on to exhibit a severe disease (FIG. 5B). Finally, we observed no statistical correlation between days of onset and the presence of ISG+ cell populations (FIG. 12A). These elements would seem to argue against a simple temporal relationship between mild/moderate and severe states and led us to investigate a systemic etiology for this split in states in serum.

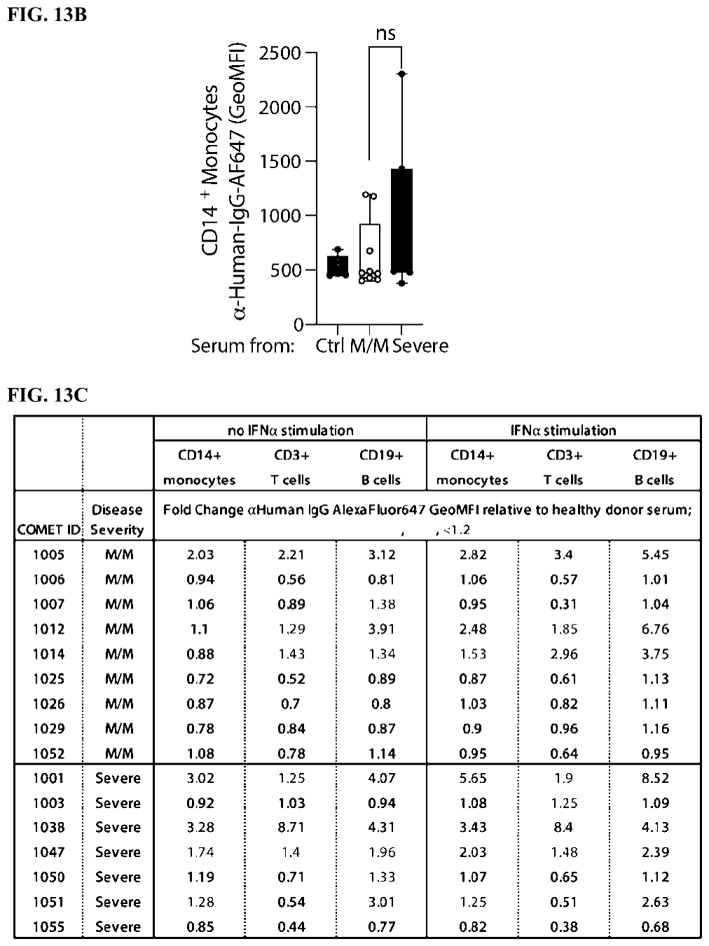

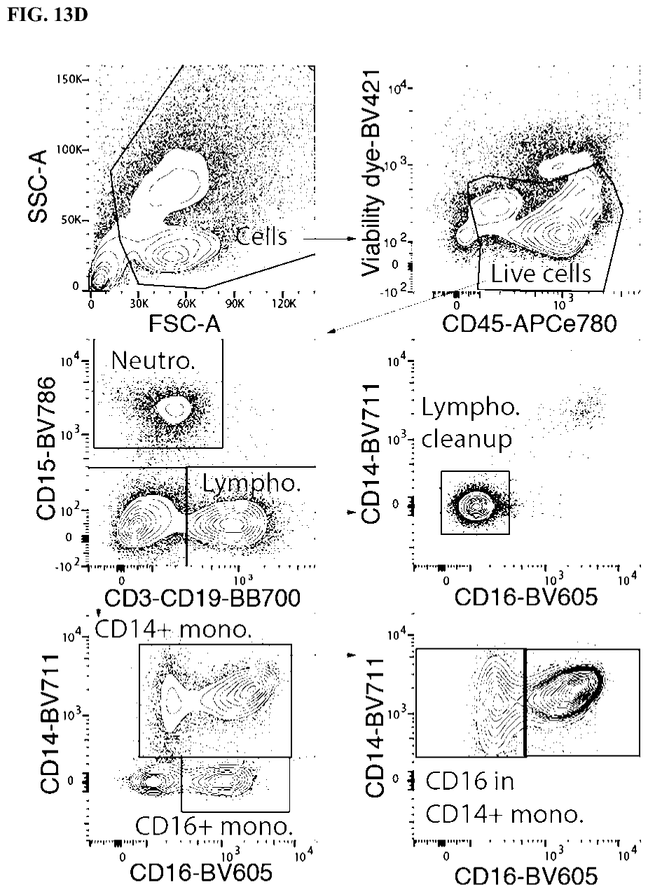

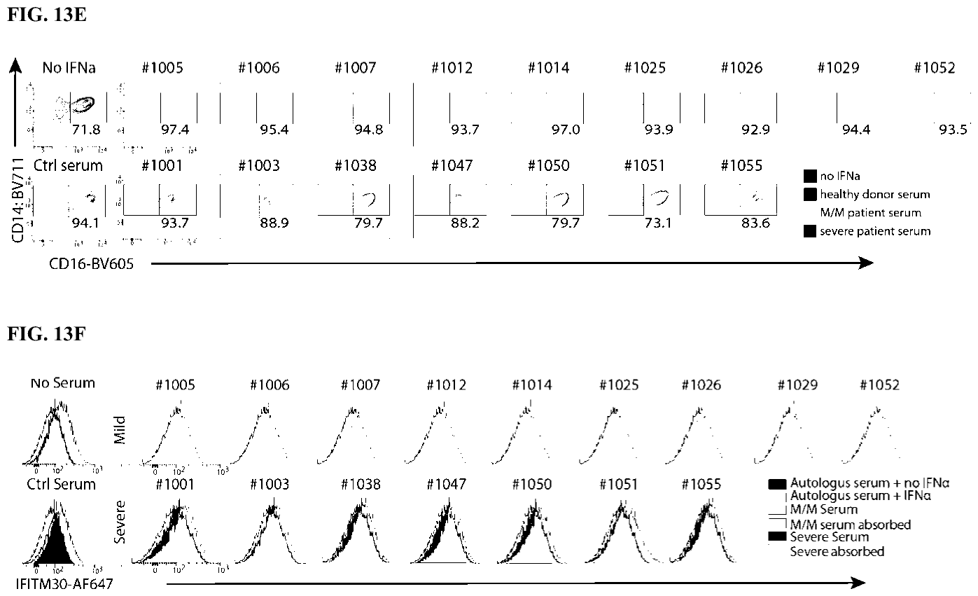

Considering this enhancement of antibodies, we first asked whether serum from severe patients also contained antibodies against ISG-expressing cells by directly applying serum to peripheral blood mononuclear cells (PBMCs from heathy individuals) cultured with and without IFNα (FIG. 13A-13D). We observed serum IgG binding from 2 mild/moderate and 2 severe COVID-19 patient (FIG. 13A). However, staining was highly variable on different cell types (FIG. 13B-13C), both with and without prior IFNα stimulation, suggesting that patients may each have unique combinations of specificities. For instance, examining patient 1050 whose serum did not stain ISG-differentiated cells directly, we found evidence of antibodies to IFNα (right inset, FIG. 3E), consistent with a very recent study (17) that also found these in approximately 12% of COVID patients. This patient was unique in our cohort and IFNα reactivity further does not explain the majority of severe patients lacking ISG cells.

We separately tested whether factors in the serum of severe patients affect the induction of the ISG signature gene pattern, using IFITM3 as a marker, in response to culture with IFNα. We thus mixed patient serum at 5%, into an IFNα stimulation of healthy PBMCs and found that, whereas control serum or serum from mild/moderate patients had no effect on differentiation as measured by either IFITM3 level or the frequencies of CD14⁺CD16⁺ intermediate monocytes produced, all severe patient serum tested had profound effects, varying from complete block to partial inhibition (FIG. 3E-3G, FIG. 13D-13E).

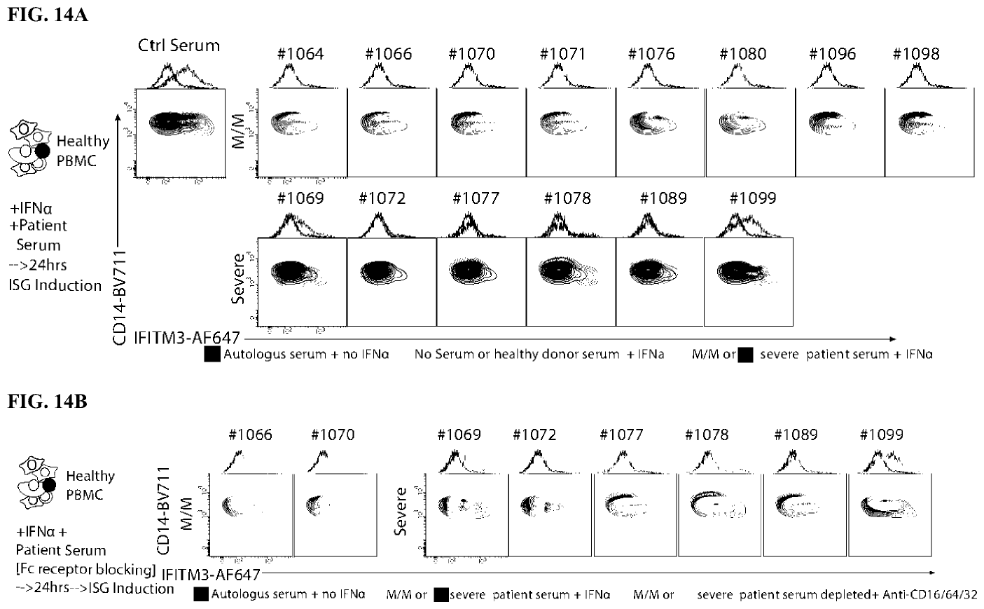

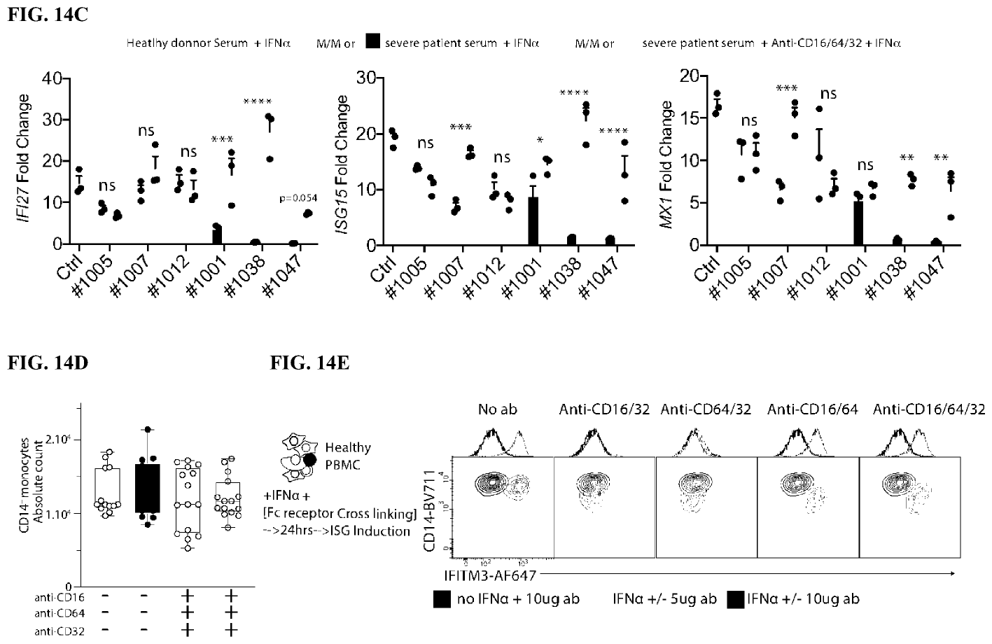

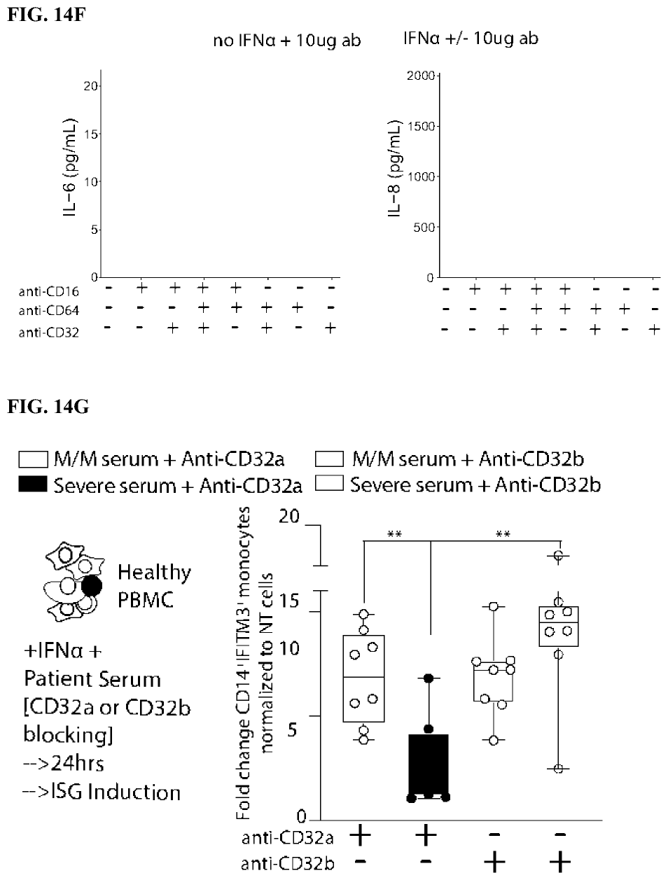

To test if antibodies in severe patient serum were responsible for this inhibition of IFNα response, we pre-adsorbed patients' sera with Protein A/G beads to deplete them. This relieved the block in both IFITM3 induction and the total yield of interferon-stimulated monocytes (FIG. 3F-3G). A similar block and release through antibody-absorption was observed for IFNα-dependent ISG signature generation in other populations including lymphocytes (FIG. 3H, FIG. 13F). We consider it likely, since profound IFN responses are dependent on a positive feedback loop from initial Interferon a Receptor (IFNAR) signaling (18), that IFN response in lymphocytes benefits from IFNAR signaling amplification in monocytes. We also confirmed an inhibition of ISG cell population generation by severe serum in a second validation cohort composed of 8 M/M and 6 severe patients (FIG. 14A, Table 3).

TABLE 3
COVID-19 Whole Blood Validation Study Cohort. Patients were enrolled as
described in Material and Methods and blood was collected from 4:30 AM rounds on the first day
after admission. Data on individuals is shown along with average and standard deviations.

| | SARS- | | | Gender | | | Days under | |
|---|---|---|---|---|---|---|---|---|
| | CoV-2 | Disease | | (1 = MALE, | Other | ICU during | mechanical | |
| COMET ID | Status | Severity | Age | 2 = F) | Infection(s) | hospital stay | Ventilation | Discharged |
| 1064 | POS | Mild/Moderate | 44 | 1 | None | No | 0 | Yes |
| 1066 | POS | Mild/Moderate | 38 | 1 | None | No | 0 | Yes |
| 1070 | POS | Mild/Moderate | 40 | 1 | Bacterial | No | 0 | Yes |
| 1071 | POS | Mild/Moderate | 35 | 2 | None | No | 0 | Yes |
| 1076 | POS | Mild/Moderate | 46 | 1 | None | No | 0 | Yes |
| 1080 | POS | Mild/Moderate | 73.2 | 1 | None | No | 0 | Yes |
| 1096 | POS | Mild/Moderate | 48 | 1 | None | No | 0 | Yes |
| 1098 | POS | Mild/Moderate | 42 | 1 | none | No | 0 | Yes |
| COVID | Average | | 46 | 1.2 | | | | |
| Positive Mild | Data: | | | | | | | |
| n = 11 | SD: | | 12 | | | | | |
| 1069 | POS | Severe | 88 | 2 | none | Yes | 0 | No |
| 1072 | POS | Severe | 47 | 1 | none | Yes | 21 | Yes |
| 1077 | POS | Severe | 54 | 1 | Bacterial | Yes | 8 | Yes |
| 1078 | POS | Severe | 43 | 1 | none | Yes | 15 | Yes |
| 1089 | POS | Severe | 30 | 2 | Bacterial | Yes | 16 | No |
| 1099 | POS | Severe | 36 | 1 | none | Yes | ND | Yes |
| COVID | Average | | 50 | 1.3 | | | | |
| Positive Severe | Data: | | | | | | | |
| n = 10 | SD: | | 21 | | | | | |

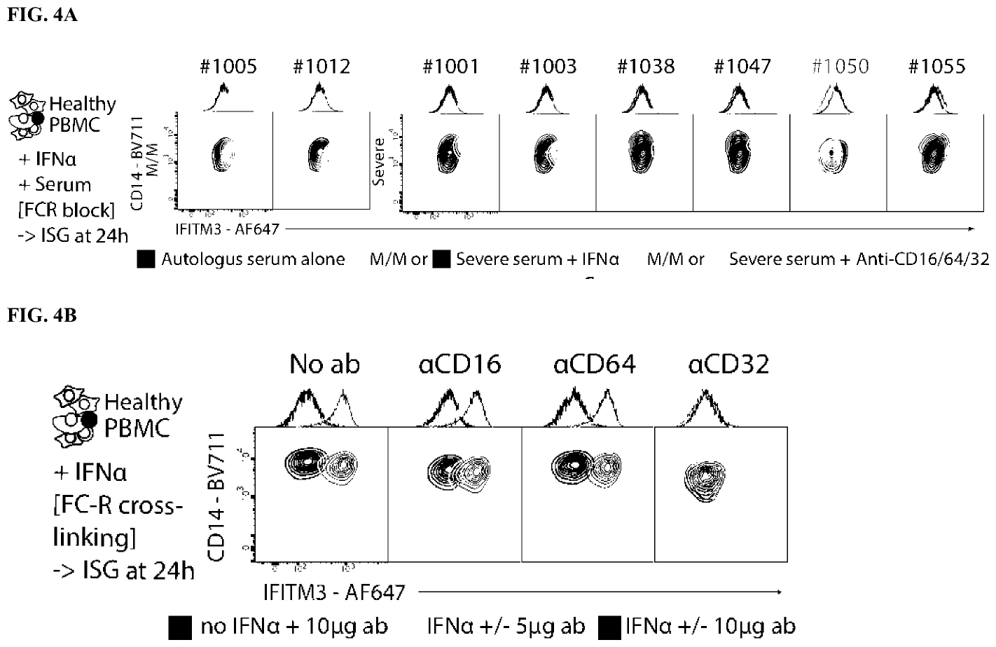

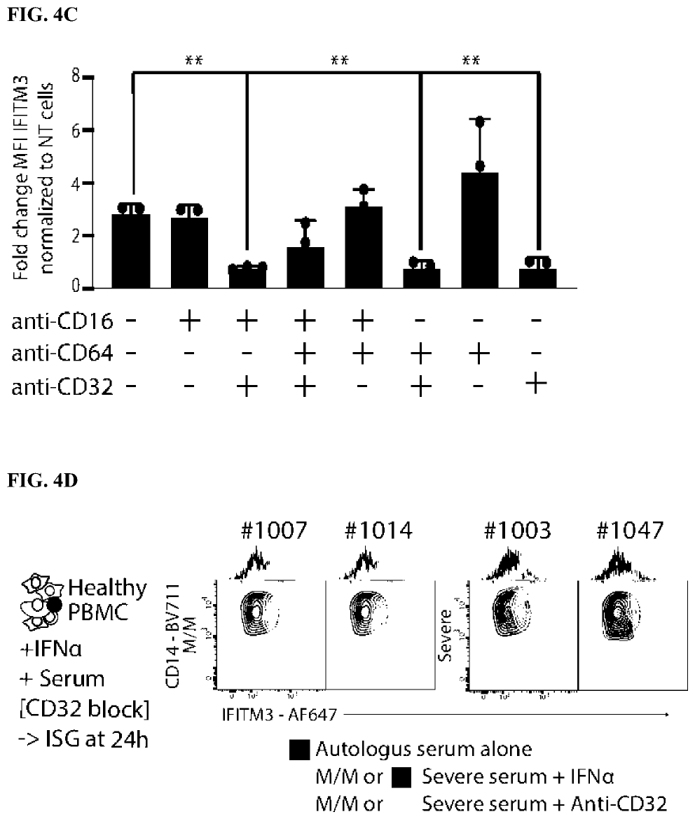

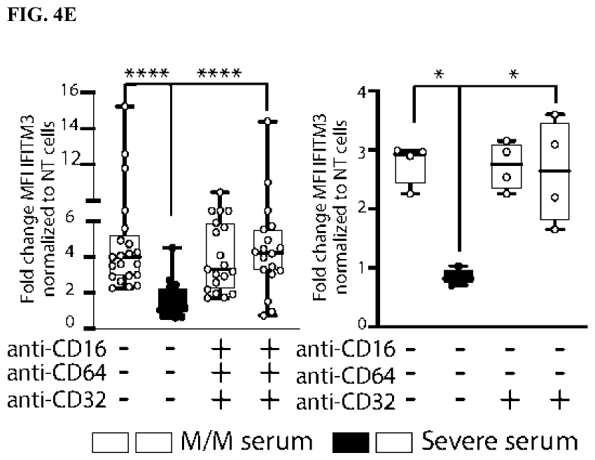

Probing the mechanism for this result, we found that blocking antibodies to Fc Receptors (CD16/CD64/CD32) during culture with IFNα and patient serum restored IFITM3 induction in cells cultured with serum from severe patient both in discovery (FIG. 4A/4E, left) and validation (FIG. 14B) cohorts. Fc receptor blocking restored not only IFITM3 induction but other ISG's, such as IFI27, ISG15, MX1 (FIG. 14C). These results and the absence of augmented cell death in PBMC cultured with serum from severe patient (FIG. 14D) suggested that antibodies present in serum from severe patients trigger Fc receptor signaling, which inhibits transcriptional responses following IFNAR engagement.

We considered that such a mechanism might represent a fundamental way for antibody generation to downregulate an interferon cascade and therefore we tested whether Fc receptor activation via cross linking antagonized IFITM3 induction by IFNα. PBMC subjected to individual crosslinking of CD32, but not CD16 or CD64, demonstrated dramatically less IFITM3 induction (FIG. 4B-4C, FIG. 14E) while crosslinking of all FcR together induced pro-inflammatory cytokine production (FIG. 14F).

Returning to severe COVID-19 serum effects, we found that blocking CD32 alone restored IFITM3 induction in PBMC's cultured with IFNα in the presence of severe serum (FIG. 4D-4E, right). Previous studies demonstrated that FcγRIIb (CD32b) blockade could lead to IFN-like responses in dendritic cells and monocytes (19) while binding of the activating Fc receptor FcγRIIa (CD32a) elicit viral immunity (20). Consistent with those previous studies, we found that blocking of FCγRIIb but not FCγRIIa, rescued IFITM3 induction in monocytes cultured with serum from severe patients (FIG. 14G).

Taken together, inhibition of a phenotype of ISG-expressing immune populations in severe patients correspond to antagonism of IFNAR signaling via FCγRIIb receptor signaling by their antibodies. In our cohort, this general antibody-mediated effect manifests in almost all severe patients, whereas antibodies against the cytokine IFNα itself were seen only in one of seven patients, and those antibodies blocked ISG function but not via FcRs (FIG. 4A). With regard to specificity, it is notable that very recent works have highlighted autoantibodies in COVID-19 binding to targets as diverse as phospholipids (21) and endothelial proteins (22) but that not all patients had developed each specificity. Our work likewise found antibody binding to mixtures of immune cells themselves and it is possible that, in the course of an infection, incomplete tolerance in the B cell compartment may include recognition of a great many host proteins including those on immune cells. While it will be important to study the likely diverse nature of antibody specificities in COVID-19, afucosylation of antibodies, which modifies selectivity for FcR subtypes, as well as differential IgG subclass selectivity is also emerging as a distinguishing feature (23) and we speculate that variable levels of these IgG subclasses in sera combined with varying affinities for different Fc receptors could result in stronger signaling through inhibitory FCγRIIb. Further work will be necessary to characterize the relative contributions of these IgG subclasses and their specificities. Regardless, our study suggests that this global targeting of ISG archetypes might be addressable with drugs such as rituximab to reduce B cell responses (24) perhaps in the presence of convalescent serum, through introduction of IVIG to compete with serum antibodies for FcR engagement (25), or with rapid development of antibodies that clinically block FCγRIIb.

### REFERENCES

1. D. Mathew et al., Deep immune profiling of COVID-19 patients reveals distinct immunotypes with therapeutic implications. Science 369, (2020).
2. J. Schulte-Schrepping et al., Severe COVID-19 Is Marked by a Dysregulated Myeloid Cell Compartment. Cell 182, 1419-1440 e1423 (2020).
3. J. Hadjadj et al., Impaired type I interferon activity and inflammatory responses in severe COVID-19 patients. Science 369, 718-724 (2020).
4. R. Zilionis et al., Single-Cell Transcriptomics of Human and Mouse Lung Cancers Reveals Conserved Myeloid Populations across Individuals and Species. Immunity 50, 1317-1334 e1310 (2019).
5. C. Trapnell et al., The dynamics and regulators of cell fate decisions are revealed by pseudotemporal ordering of single cells. Nat Biotechnol 32, 381-386 (2014).
6. I. C. Huang et al., Distinct patterns of IFI™-mediated restriction of filoviruses, SARS coronavirus, and influenza A virus. PLOS Pathog 7, e1001258 (2011).
7. M. Reyes et al., An immune-cell signature of bacterial sepsis. Nat Med 26, 333-340 (2020).
8. B. Engelmann, S. Massberg, Thrombosis as an intravascular effector of innate immunity. Nat Rev Immunol 13, 34-45 (2013).
9. S. Cui, S. Chen, X. Li, S. Liu, F. Wang, Prevalence of venous thromboembolism in patients with severe novel coronavirus pneumonia. J Thromb Haemost 18, 1421-1424 (2020).
10. P. Davizon-Castillo, J. W. Rowley, M. T. Rondina, Megakaryocyte and Platelet Transcriptomics for Discoveries in Human Health and Disease. Arterioscler Thromb Vasc Biol 40, 1432-1440 (2020).
11. K. D. Mason et al., Programmed anuclear cell death delimits platelet life span. Cell 128, 1173-1186 (2007).
12. D. Bongiovanni et al., Transcriptome Analysis of Reticulated Platelets Reveals a Prothrombotic Profile. Thrombosis and haemostasis 119, 1795-1806 (2019).
13. W. S. Chen et al., Uncovering axes of variation among single-cell cancer specimens. Nat Methods 17, 302-310 (2020).
14. E. Pujadas et al., SARS-COV-2 viral load predicts COVID-19 mortality. Lancet Respir Med 8, e70 (2020).
15. S. Hue et al., Uncontrolled Innate and Impaired Adaptive Immune Responses in Patients with COVID-19 ARDS. Am J Respir Crit Care Med, (2020).
16. Y. Wang et al., Kinetics of viral load and antibody response in relation to COVID-19 severity. J Clin Invest 130, 5235-5244 (2020).
17. P. Bastard et al., Auto-antibodies against type I IFNs in patients with life-threatening COVID-19. Science, eabd4585 (2020).
18. A. Michalska, K. Blaszczyk, J. Wesoly, H. A. R. Bluyssen, A Positive Feedback Amplifier Circuit That Regulates Interferon (IFN)-Stimulated Gene Expression and Controls Type I and Type II IFN Responses. Front Immunol 9, 1135 (2018).
19. K. M. Dhodapkar et al., Selective blockade of the inhibitory Fcgamma receptor (FcgammaRIIB) in human dendritic cells and monocytes induces a type I interferon response program. J Exp Med 204, 1359-1369 (2007).
20. S. Bournazos, D. Corti, H. W. Virgin, J. V. Ravetch, Fc-optimized antibodies elicit CD8 immunity to viral respiratory infection. Nature, (2020).
21. Y. Zuo et al., Prothrombotic autoantibodies in serum from patients hospitalized with COVID-19. Science translational medicine 12, eabd3876 (2020).
22. C. R. Consiglio et al., The Immunology of Multisystem Inflammatory Syndrome in Children with COVID-19. Cell 183, 968-981 e967 (2020).
23. S. Chakraborty et al., Proinflammatory IgG Fc structures in patients with severe COVID-19. Nat Immunol, (2020).
24. H. Huang, C. Benoist, D. Mathis, Rituximab specifically depletes short-lived autoreactive plasma cells in a mouse model of inflammatory arthritis. Proc Natl Acad Sci USA 107, 4658-4663 (2010).
25. R. J. Looney, J. Huggins, Use of intravenous immunoglobulin G (IVIG). Best Pract Res Clin Haematol 19, 3-25 (2006).
26. D. Bongiovanni et al., Transcriptome Analysis of Reticulated Platelets Reveals a Prothrombotic Profile. Thrombosis and haemostasis 119, 1795-1806 (2019).
27. T. Stuart et al., Comprehensive Integration of Single-Cell Data. Cell 177, 1888-1902 e1821 (2019).
28. D. Dominguez et al., A high-resolution transcriptome map of cell cycle reveals novel connections between periodic genes and cancer. Cell research 26, 946-962 (2016).
29. H. M. Kang et al., Multiplexed droplet single-cell RNA-sequencing using natural genetic variation. Nat Biotechnol 36, 89-94 (2018).
30. C. Genomes Project et al., A global reference for human genetic variation. Nature 526, 68-74 (2015).
31. H. Li, A statistical framework for SNP calling, mutation discovery, association mapping and population genetical parameter estimation from sequencing data. Bioinformatics 27, 2987-2993 (2011).
32. C. Hafemeister, R. Satija, Normalization and variance stabilization of single-cell RNA-seq data using regularized negative binomial regression. Genome biology 20, 296 (2019).
33. C. S. McGinnis, L. M. Murrow, Z. J. Gartner, DoubletFinder: Doublet Detection in Single-Cell RNA Sequencing Data Using Artificial Nearest Neighbors. Cell Syst 8, 329-337 e324 (2019).
34. I. Korsunsky et al., Fast, sensitive and accurate integration of single-cell data with Harmony. Nat Methods 16, 1289-1296 (2019).
35. X. Qiu et al., Reversed graph embedding resolves complex single-cell trajectories. Nat Methods 14, 979-982 (2017).
36. X. Qiu et al., Single-cell mRNA quantification and differential analysis with Census. Nat Methods 14, 309-315 (2017).
37. C. Trapnell et al., The dynamics and regulators of cell fate decisions are revealed by pseudotemporal ordering of single cells. Nat Biotechnol 32, 381-386 (2014).
38. W. S. Chen et al., Uncovering axes of variation among single-cell cancer specimens. Nat Methods 17, 302-310 (2020).
39. K. R. Moon et al., Visualizing structure and transitions in high-dimensional biological data. Nat Biotechnol 37, 1482-1492 (2019).
40. C. R. Zamecnik et al., ReScan, a Multiplex Diagnostic Pipeline, Pans Human Sera for SARS-COV-2 Antigens. medRxiv, 2020.2005.2011.20092528 (2020).
41. A. Leligdowicz et al., Validation of two multiplex platforms to quantify circulating markers of inflammation and endothelial injury in severe infection. PLOS One 12, e0175130 (2017).

1. A method of abrogating severity of a viral infection that induces interferon in a subject in need thereof comprising administering to the subject in need thereof a pharmaceutical composition comprising: (a) a therapeutically effective amount of a CD32b antagonist or agonist; and (ii) and one or a plurality of pharmaceutically acceptable carriers.

2.-3. (canceled)

4. The method of claim 1 of claim 1, wherein the viral infection is a human coronavirus 229E (HCoV-229E) infection, human coronavirus OC43 (HCoV-OC43) infection, severe acute respiratory syndrome coronavirus (SARS-COV), human coronavirus NL63 (HCoV-NL63), human coronavirus HKUI, Middle East respiratory syndrome-related coronavirus (MERS-CoV), or severe acute respiratory syndrome coronavirus 2 (SARS-COV-2) infection.

5.-7. (canceled)

8. The method of claim 1, wherein the CD32b antagonist is an anti-CD32b antibody or antigen-binding fragment thereof.

9. (canceled)

10. The method of claim 8, wherein the humanized monoclonal antibody or antigen-binding fragment thereof comprises at least one CDR identified in Table 1 or at least one CDR comprising at least about 70% sequence identity to a CDR sequence identified in Table 1.

11. The method of claim 10, wherein the humanized monoclonal antibody or antigen binding fragment thereof comprises two or three CDRs identified in Table 1, or two or three CDRs comprising at least about 70% sequence identity to a CDR sequence identified in Table 1.

12.-19. (canceled)

20. The method of claim 1 further comprising an active agent that reduces total plasma cell or B cell counts.

21-23. (canceled)

24. A method of treating Coronaviridae infection in a subject in need thereof comprising administering to the subject in need thereof a therapeutically effective amount of a pharmaceutical composition comprising: (i) a CD32b antagonist; and (ii) one or a plurality of pharmaceutically acceptable carriers.

25.-26. (canceled)

27. The method of claim 24, wherein the viral infection is a human coronavirus 229E (HCoV-229E) infection, human coronavirus OC43 (HCoV-OC43) infection, severe acute respiratory syndrome coronavirus (SARS-COV), human coronavirus NL63 (HCoV-NL63), human coronavirus HKUI, Middle East respiratory syndrome-related coronavirus (MERS-CoV), or severe acute respiratory syndrome coronavirus 2 (SARS-COV-2) infection.

28.-31. (canceled)

32. The method of claim 24, wherein the anti-CD32b antibody or antigen-binding fragment thereof is a humanized monoclonal antibody or antigen-binding fragment thereof.

33. (canceled)

34. The method of claim 32, wherein the humanized monoclonal antibody or antigen binding fragment thereof comprises two or three CDRs identified in Table 1, or two or three CDRs comprising at least about 70% sequence identity to a CDR sequence identified in Table 1.

35.-41. (canceled)

42. The method of claim 24, wherein the CD32b antagonist is administered intravenously, intramuscularly, topically intradermally, transmucosally, subcutaneously, sublingually, orally, intravaginally, intraocularly, intranasally, intrarectally, gastrointesinally, intraductally, inthecally, subdurally, exradurally, intraventricularly, intraarticuarly, intraperitoneally, or into the pleural cavity.

43. The method of claim 24 further comprising an active agent that reduces total plasma cell or B cell counts.

44.-45. (canceled)

46. The method of claim 24, wherein the antibody or antibody-binding fragment is multivalent IgG1 molecule or an IgG3 molecule.

47. A method of treating Coronaviridae infection in a subject in need thereof, said method comprising administering to the subject in need thereof a therapeutically effective amount of a CD32b antagonist or agonist and one or a plurality of pharmaceutically acceptable carriers.

48.-49. (canceled)

50. The method of claim 47, wherein the viral Coronaviridae infection is a human coronavirus 229E (HCoV-229E) infection, human coronavirus OC43 (HCoV-OC43) infection, severe acute respiratory syndrome coronavirus (SARS-COV), human coronavirus NL63 (HCoV-NL63), human coronavirus HKUI, Middle East respiratory syndrome-related coronavirus (MERS-COV), or severe acute respiratory syndrome coronavirus 2 (SARS-COV-2) infection.

51.-53. (canceled)

54. The method of claim 47, wherein the CD32b antagonist is an anti-CD32b antibody or antigen-binding fragment thereof.

55.-57. (canceled)

58. The method of claim 47, wherein the CD32b antagonist is a humanized anti-CD32b antibody or antigen-binding fragment thereof comprising:
a. a VH region comprising at least about 70% sequence identity to a variable heavy sequence identified in Table 1; and
b. a VL region comprising at least about 70% sequence identity to a variable light chain identified in Table 1.

59.-64. (canceled)

65. The method of claim 47, wherein the method comprises administering a therapeutically effective amount of a CD32b antagonist; and wherein the CD32b antagonist is administered intravenously, intramuscularly, topically intradermally, transmucosally, subcutaneously, sublingually, orally, intravaginally, intraocularly, intranasally, intrarectally, gastrointesinally, intraductally, inthecally, subdurally, exradurally, intraventricularly, intraarticuarly, intraperitoneally, or into the pleural cavity.

66.-69. (canceled)

70. A method of preventing acute respiratory distress syndrome in a subject in need thereof comprising administering to the subject in need thereof a pharmaceutical composition comprising: (i) a therapeutically effective amount of a CD32b antagonist or agonist; and (ii) one or a plurality of pharmaceutically acceptable carriers.

71.-72. (canceled)

73. The method of claim 70, wherein the acute respiratory distress syndrome is caused by a human coronavirus 229E (HCoV-229E), human coronavirus OC43 (HCoV-OC43), severe acute respiratory syndrome coronavirus (SARS-COV), human coronavirus NL63 (HCoV-NL63), human coronavirus HKUI, Middle East respiratory syndrome-related coronavirus (MERS-COV), or severe acute respiratory syndrome coronavirus 2 (SARS-COV-2).

74.-75. (canceled)

76. The method of claim 70, wherein the method comprises administering a CD32b antagonist and wherein the CD32b antagonist is an anti-CD32b antibody or antigen-binding fragment thereof.

77.-116. (canceled)

117. A method of inducing secretion of interferon-alpha (IFN-a) in a subject in need thereof comprising administering to the subject in need thereof a pharmaceutical composition comprising: (i) a therapeutically effective amount of a CD32b antagonist or agonist; and (ii) one or a plurality of pharmaceutically acceptable carriers.

118.-135. (canceled)

136. A method of treating acute lung injury in a subject in need thereof administering to the subject in need thereof a pharmaceutical composition comprising: (i) a therapeutically effective amount of a CD32b antagonist or agonist; and (ii) one or a plurality of pharmaceutically acceptable carriers.

137.-143. (canceled)

144. The method of claim 136, wherein the humanized monoclonal antibody or antigen binding fragment thereof comprises two or three CDRs identified in Table 1, or two or three comprising at least about 70% sequence identity to a CDR sequence identified in Table 1.

145.-156. (canceled)
